(19) United States
(12) Patent Application Publication
(10) Pub. No.: US 2024/0140922 A1
(43) Pub. Date: May 2, 2024

(54) FUSED HETEROBICYCLIC ANTIVIRAL AGENTS

(71) Applicant: ENANTA PHARMACEUTICALS, INC., WATERTOWN, MA (US)

(72) Inventors: Samuel Bartlett, Brighton, MA (US); Joseph D. Panarese, Newton, MA (US); Sourav Ghorai, Waltham, MA (US); Nathaniel Thomas Kenton, Watertown, MA (US); Sean Rafferty, Watertown, MA (US); Jonathan Thielman, Cambridge, MA (US); Peilin Xu, Watertown, MA (US); Bin Wang, Newton, MA (US); William Cassels, Watertown, MA (US); Scott Mitchell, Woburn, MA (US); Yat Sun Or, Waltham, MA (US)

(21) Appl. No.: 18/236,560

(22) Filed: Aug. 22, 2023

Related U.S. Application Data
(60) Provisional application No. 63/399,971, filed on Aug. 22, 2022.

(51) Int. Cl. C07D 285/36; A61P 31/20; C07D 337/08; C07D 417/04; C07D 417/08; C07D 417/12; C07D 417/14; C07D 487/04; C07D 513/04; C07F 9/6536

(52) U.S. Cl. CPC C07D 285/36; A61P 31/20; C07D 337/08; C07D 417/04; C07D 417/08; C07D 417/12; C07D 417/14; C07D 487/04; C07D 513/04; C07F 9/6536

(57) ABSTRACT

The present invention discloses compounds of Formula (I), or pharmaceutically acceptable salts, thereof:

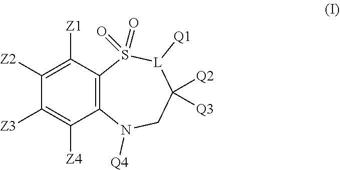

which inhibit the cellular entry of hepatitis B virus (HBV) and/or hepatitis D virus (HDV) or interfere with the function of the life cycle of HBV and/or HDV and are also useful as antiviral agents. The present invention further relates to pharmaceutical compositions comprising the aforementioned compounds for administration to a subject suffering from HBV and/or HDV infection. The invention also relates to methods of treating an HBV and/or HDV infection in a subject by administering a pharmaceutical composition comprising the compounds of the present invention.

### RELATED APPLICATION

This application claims the benefit of U.S. Provisional Application No. 63/399,971, filed on Aug. 22, 2022. The entire teachings of the above application are incorporated herein by reference.

### BACKGROUND OF THE INVENTION

The hepatitis delta viruses, or HDV, are eight species of negative-sense single-stranded RNA viruses (or virus-like particles) classified together as the genus Deltavirus, within the realm Ribozyviria. The HDV virion is a small, spherical, enveloped particle with a 36 nm diameter; its viral envelope contains host phospholipids, as well as three proteins taken from the hepatitis B virus—the large, medium, and small hepatitis B surface antigens. This assembly surrounds an inner ribonucleoprotein (RNP) particle, which contains the genome surrounded by hepatitis D antigen (HDAg).

The HDV genome is negative sense, single-stranded, closed circular RNA; with a genome of approximately 1700 nucleotides, HDV is the smallest virus known to infect animals. Its genome is unique among animal viruses because of its high GC nucleotide content. Its nucleotide sequence is about 70% self-complementary, allowing the genome to form a partially double-stranded, rod-like RNA structure. Millions of people throughout the world are chronically infected with hepatitis D virus (HDV). For those that are chronically infected, many will develop complications of liver disease from cirrhosis or hepatocellular carcinoma (HCC).

HBV is a member of the Hepadnavirus family, and it is able to replicate through the reverse transcription of an RNA intermediate. The 3.2-kb HBV genome exists in a circular, partially doublestranded DNA conformation (rcDNA) that has four overlapping open reading frames (ORF). These encode for the core, polymerase, envelope, and X proteins of the virus. rcDNA must be converted into covalently closed circular DNA (cccDNA) in cells prior to the transcription of viral RNAs. As rcDNA is transcriptionally inert, cccDNA is the only template for HBV transcription, and its existence is required for infection.

The HBV viral envelope contains a mixture of surface antigen proteins (HBsAg). The HBsAg coat contains three proteins that share a common region that includes the smallest of the three proteins (SHBsAg). The other two proteins, Medium HBsAg (MHBsAg) and Large HBsAg (LHBsAg), both contain a segment of SHBsAg with additional polypeptide segments. SHBsAg, MHBsAg, and LHBsAg can also assemble into a non-infectious subviral particle known as the 22-nm particle that contains the same proteins found around infectious viral particles. As the 22-nm particles contain the same antigenic surface proteins that exist around the infectious HBV virion, they can be used as a vaccine to produce neutralizing antibodies.

HBV and HDV both gain entry into liver cells via the human NTCP bile acid transporter. Viral particles recognize their receptor via the N-terminal domain of the large hepatitis B surface antigen, HBsAg. After entering the hepatocyte, the virus is uncoated and the nucleocapsid translocated to the nucleus thereby infecting the cell.

There is a need in the art for novel therapeutic agents that treat, ameliorate or prevent HBV and/or HDV infection. Administration of these therapeutic agents to an HBV and/or HDV infected patient, either as monotherapy or in combination with other HBV and/or HDV treatments or ancillary treatments, will lead to significantly improved prognosis, diminished progression of the disease, and enhanced seroconversion rates.

### SUMMARY OF THE INVENTION

The present invention relates to novel antiviral compounds, pharmaceutical compositions comprising such compounds, as well as methods to treat or prevent viral (particularly HBV and/or HDV) infection in a subject in need of such therapy with said compounds. Compounds of the present invention inhibit the entry of HBV and/or HDV or interfere with the life cycle of HBV and/or HDV and are also useful as antiviral agents. In addition, the present invention provides processes for the preparation of said compounds.

The present invention provides compounds represented by Formula (I),

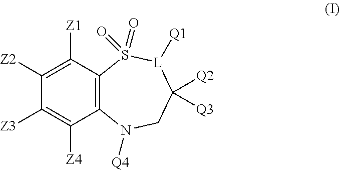

and pharmaceutically acceptable salts, N-oxides, esters and prodrugs thereof, wherein:
Q1, Q2, Q3, and Q4 are each independently selected from hydrogen, optionally substituted —C₁-C₆ alkyl, optionally substituted —C₂-C₆ alkenyl, optionally substituted —C₁-C₆ alkoxy, optionally substituted —C₃-C₅ cycloalkyl, optionally substituted —C₃-C₈ cycloalkenyl, optionally substituted 3- to 8-membered heterocycloalkyl, optionally substituted aryl, and optionally substituted heteroaryl;

Alternatively, Q1 and Q2, or Q1 and Q3 are taken together with the atoms to which they are attached to form an optionally substituted 3-8 membered heterocyclic or carbocyclic ring containing 0, 1, 2, or 3 double bonds;

Alternatively, Q2 and Q3 are taken together with the carbon atom to which they are attached to form an optionally substituted 3-8 membered heterocyclic or carbocyclic ring containing 0, 1, 2, or 3 double bonds;
L is CR₁₄ or N;
Z1, Z3, and Z4 are each independently selected from:
1) hydrogen;
2) halogen;
3) —NO₂;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₈ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁₁)S(O)₂(R₁₂);
21) —N(R₁₁)(R₁₂);
22) —N(R₁₃)C(O)N(R₁₁)(R₁₂);
23) —N(R₁₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
   Z2 is selected from:
1) Optionally substituted —C₃-C₈ cycloalkyl;
2) Optionally substituted 3- to 12-membered heterocycloalkyl;
3) Optionally substituted aryl;
4) Optionally substituted arylalkyl;
5) Optionally substituted heteroaryl; and
6) Optionally substituted heteroarylalkyl;
   wherein R₁₁, R₁₂, and R₁₃, are each independently selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl. Alternatively, R₁₁ and R₁₂ are taken together with the nitrogen atom to which they attached to form an optionally substituted 3-8 membered heterocyclic containing 0, 1, 2, or 3 double bonds; and
R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy.

Preferably, Z2 is optionally substituted aryl, optionally substituted heteroaryl or optionally substituted bicyclic heterocycloalkyl, more preferably Z2 is optionally substituted aryl or optionally substituted heteroaryl.

Each preferred group stated above can be taken in combination with one, any or all other preferred groups.

### DETAILED DESCRIPTION OF THE INVENTION

In one embodiment, the present invention provides a compound of Formula (I) as described above, or a pharmaceutically acceptable salt thereof.

In certain embodiments, the present invention relates to compounds of Formula (I), and pharmaceutically acceptable salts thereof, wherein Z1 is hydrogen, halogen, -Me, or —OMe.

In certain embodiments, the present invention relates to compounds of Formula (I), and pharmaceutically acceptable salts thereof, wherein Z1 is hydrogen.

In certain embodiments, the present invention relates to compounds of Formula (I), and pharmaceutically acceptable salts thereof, wherein Z2 is optionally substituted heterocycloalkyl.

In certain embodiments, the present invention relates to compounds of Formula (I), and pharmaceutically acceptable salts thereof, wherein Z2 is optionally substituted bicyclic heterocycloalkyl.

In certain embodiments, the present invention relates to compounds of Formula (I), and pharmaceutically acceptable salts thereof, wherein Z2 is optionally substituted aryl or optionally substituted heteroaryl.

In certain embodiments, the present invention relates to compounds of Formula (I), and pharmaceutically acceptable salts thereof, wherein Z2 is optionally substituted phenyl or optionally substituted pyridinyl.

In certain embodiments, the present invention relates to compounds of Formula (I), and pharmaceutically acceptable salts thereof, wherein Z3 is hydrogen, halogen, —OR₁₁, —SR₁₁, or —N(R₁₁)(R₁₂), wherein R₁₁ and R₁₂ are previously defined.

In certain embodiments, the present invention relates to compounds of Formula (I), and pharmaceutically acceptable salts thereof, wherein Z3 is —F, —Cl, —Br, —CN, —OCH₃, —S CH₃, —SCH₂CH₂, or —N(CH₃)₂.

In certain embodiments, the present invention relates to compounds of Formula (I), and pharmaceutically acceptable salts thereof, wherein Z4 is hydrogen, halogen, -Me, or —OMe.

In certain embodiments, the present invention relates to compounds of Formula (I), and pharmaceutically acceptable salts thereof, wherein Z4 is hydrogen.

In certain embodiments, the present invention relates to compounds of Formula (I), and pharmaceutically acceptable salts thereof, wherein Z1 is hydrogen, and Z4 is hydrogen.

In certain embodiments, the present invention relates to compounds of Formula (I), and pharmaceutically acceptable salts thereof, wherein L is nitrogen.

In certain embodiments, the present invention relates to compounds of Formula (I), and pharmaceutically acceptable salts thereof, wherein L is nitrogen, and Q1 is hydrogen, or optionally substituted methyl.

In certain embodiments, the present invention relates to compounds of Formula (I), and pharmaceutically acceptable salts thereof, wherein L is CR₁₄, where R₁₄ is methyl, ethyl, isopropyl or cyclopropyl.

In certain embodiments, the present invention relates to compounds of Formula (I), and pharmaceutically acceptable salts thereof, wherein L is CR₁₄, where R₁₄ is methyl, ethyl, isopropyl or cyclopropyl, and Q1 is hydrogen, or optionally substituted methyl.

In certain embodiments, the present invention relates to compounds of Formula (I), and pharmaceutically acceptable salts thereof, wherein Q2 is hydrogen, or optionally substituted methyl.

In certain embodiments, the present invention relates to compounds of Formula (I), and pharmaceutically acceptable salts thereof, wherein Q3 is optionally substituted —C₁-C₆ alkyl, optionally substituted —C₃-C₈ cycloalkyl, optionally substituted 3- to 8-membered heterocycloalkyl, optionally substituted aryl, or optionally substituted heteroaryl.

In certain embodiments, the present invention relates to compounds of Formula (I), and pharmaceutically acceptable salts thereof, wherein Q3 is —CH₂R₂₁, where R₂₁ is optionally substituted —C₁-C₅ alkyl, optionally substituted —C₂-C₈ alkenyl, optionally substituted —C₁-C₅ alkoxy, optionally substituted —C₃-C₈ cycloalkyl, optionally substituted —C₃-C₈ cycloalkenyl, optionally substituted 3- to 8-membered heterocycloalkyl, optionally substituted aryl, or optionally substituted heteroaryl.

In certain embodiments of the compounds of Formula (I), Q3 is optionally substituted phenyl, optionally substituted benzyl, optionally substituted methyl, optionally substituted ethyl, optionally substituted t-butyl, optionally substituted isopropyl, optionally substituted isobutyl, optionally substituted neopentyl,

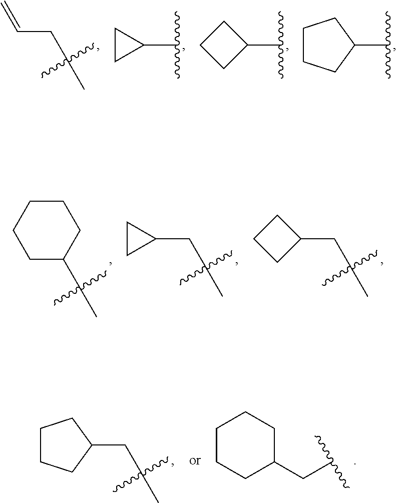

In certain embodiments, the present invention relates to compounds of Formula (I), and pharmaceutically acceptable salts thereof, wherein Q4 is optionally substituted —C₃-C₈ cycloalkyl or optionally substituted 3- to 8-membered heterocycloalkyl.

In certain embodiments, the present invention relates to compounds of Formula (I), and pharmaceutically acceptable salts thereof, wherein Q4 is optionally substituted aryl or optionally substituted heteroaryl.

In certain embodiments, the present invention relates to compounds of Formula (I), and pharmaceutically acceptable salts thereof, wherein Q4 is optionally substituted phenyl.

In certain embodiments of the compounds of Formula (I), Q4 is derived from one of the following by removal of a hydrogen atom and is optionally substituted:

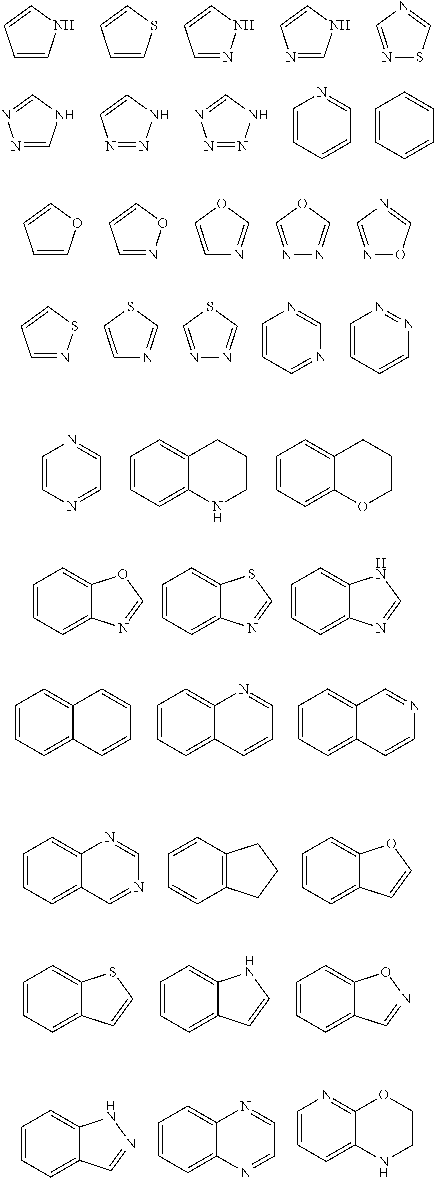

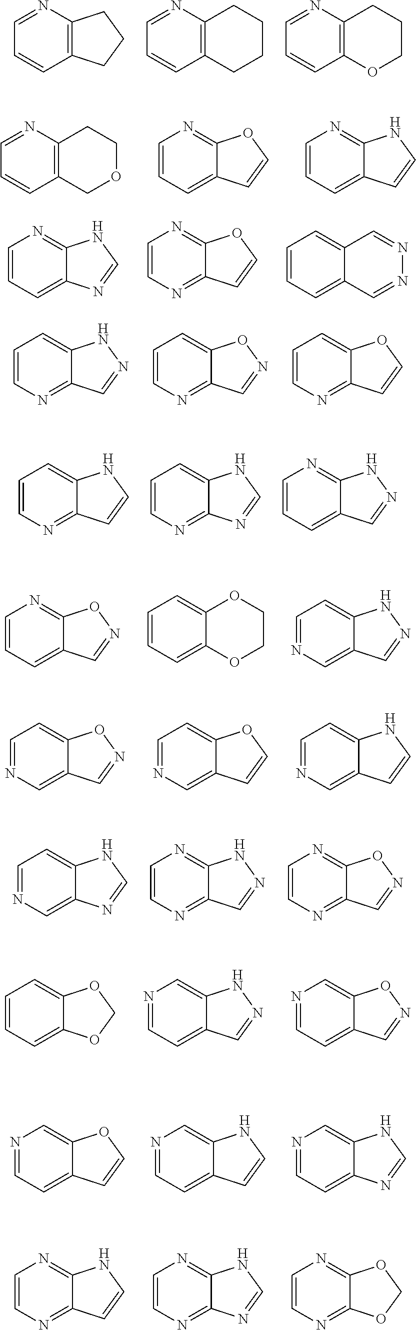

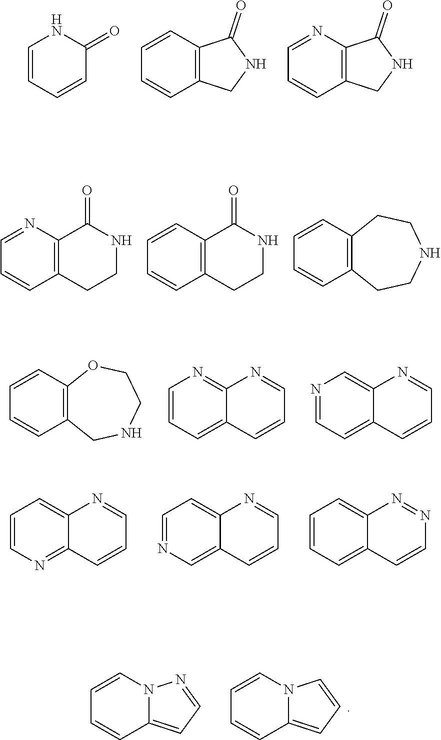

In certain embodiments of the compounds of Formula (I), Q4 is derived from one of the following by removal of a hydrogen atom and is optionally substituted:

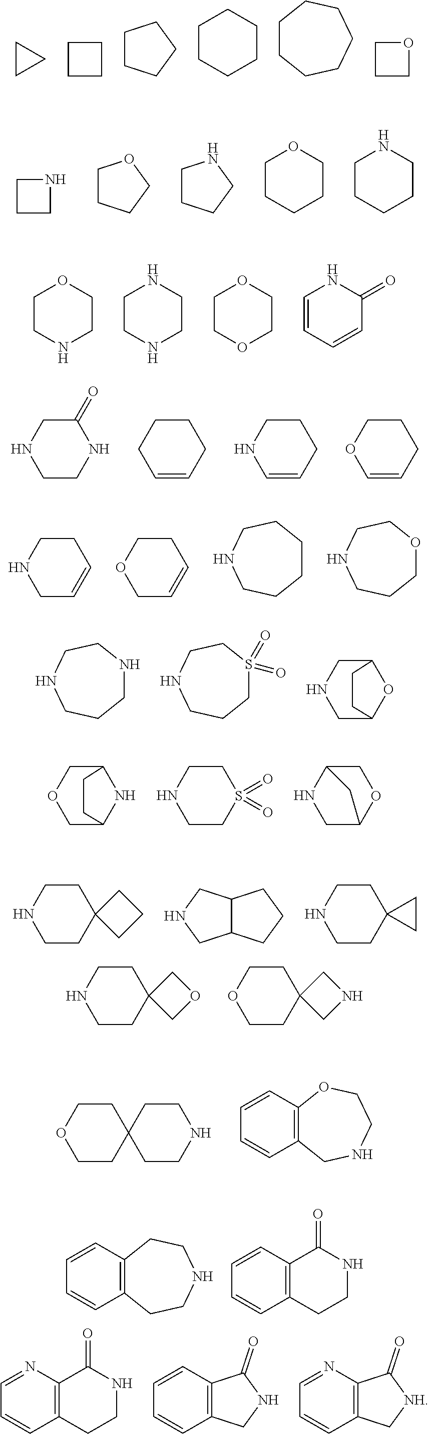

In certain embodiments of the compounds of Formula (I), Z2 is derived from one of the following by removal of a hydrogen atom and is optionally substituted:

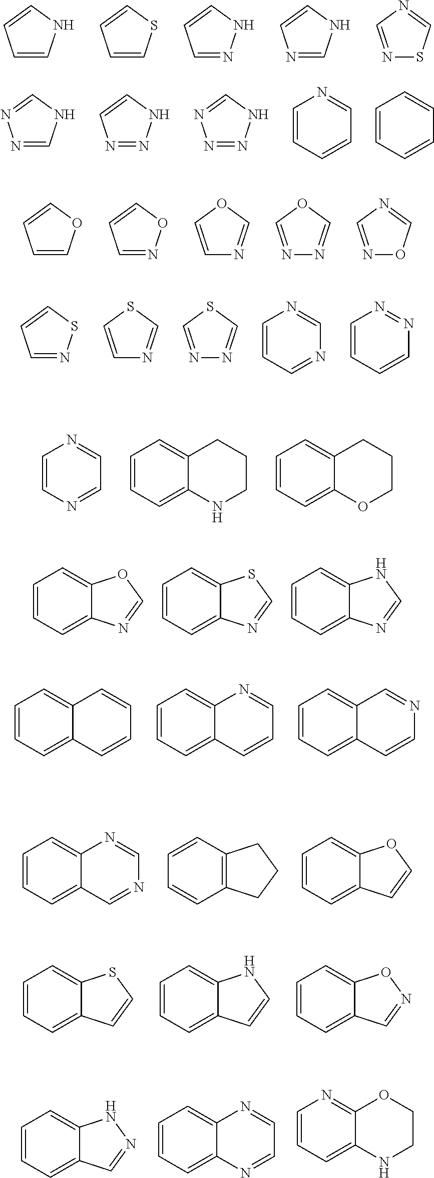

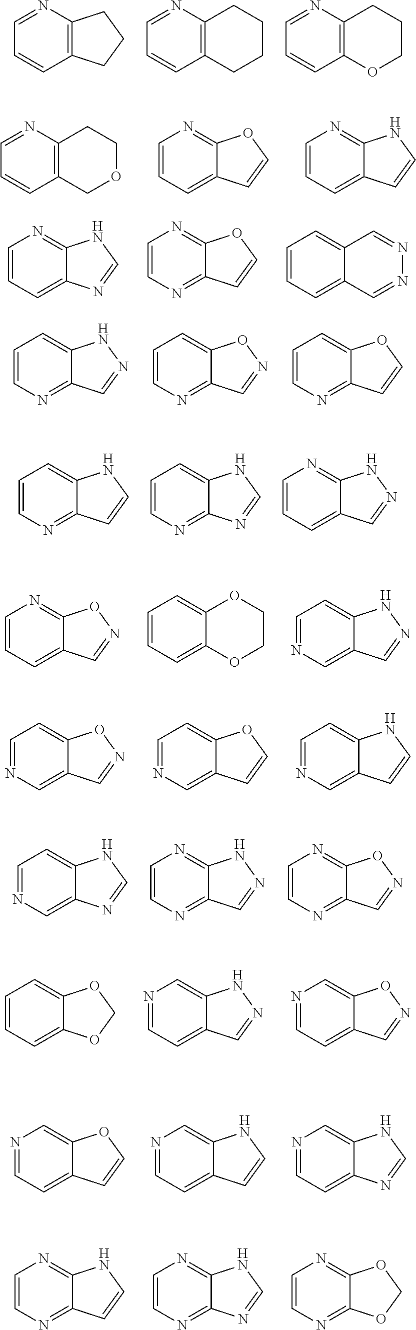

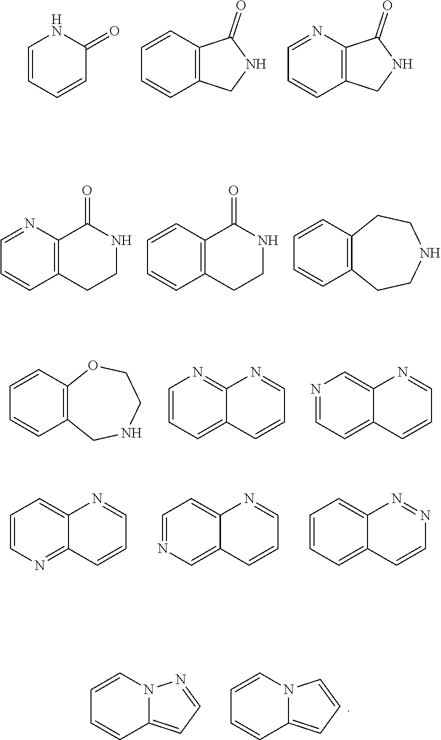

In certain embodiments of the compounds of Formula (I), Z2 is derived from one of the following by removal of a hydrogen atom and is optionally substituted:

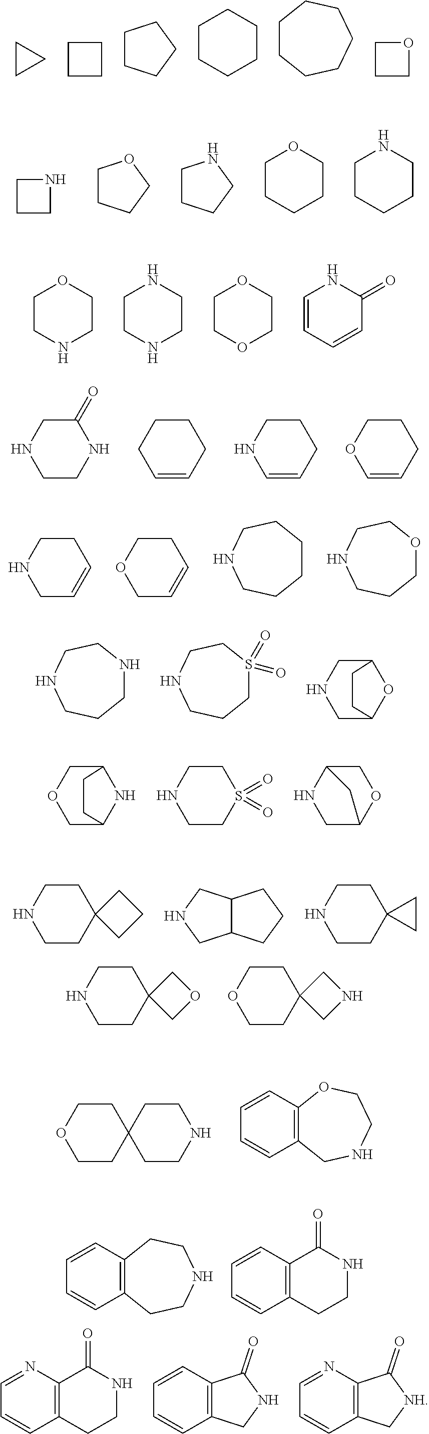

In one embodiment of the present invention, the compound of Formula (I) is represented by Formula (II),

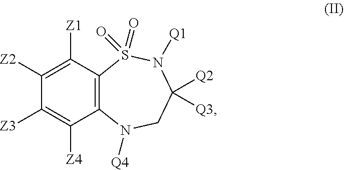

wherein Z1, Z2, Z3, Z4, Q1, Q2, Q3, and Q4 are as previously defined.

In one embodiment of the present invention, the compound of Formula (I) is represented by Formula (III),

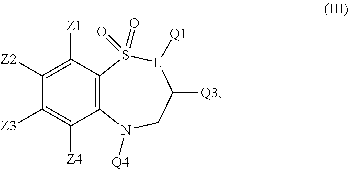

wherein Z1, Z2, Z3, Z4, L, Q1, Q3, and Q4 are as previously defined.

In one embodiment of the present invention, the compound of Formula (I) is represented by Formula (IV),

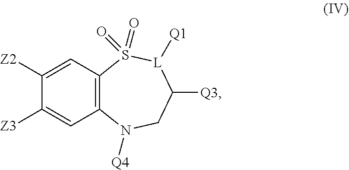

wherein Z2, Z3, L, Q1, Q3, and Q4 are as previously defined.

In one embodiment of the present invention, the compound of Formula (I) is represented by Formula (V-1) or Formula (V-2),

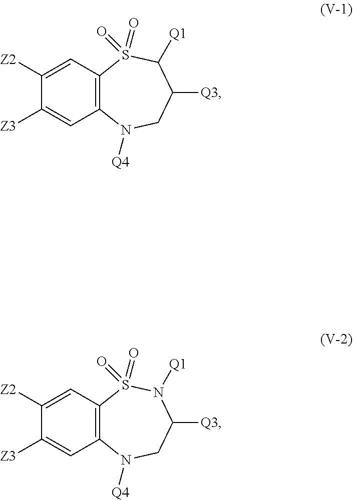

wherein Z2, Z3, Q1, Q3, and Q4 are as previously defined.

In one embodiment of the present invention, the compound of Formula (I) is represented by one of Formulae (VI-1 to VI-7),

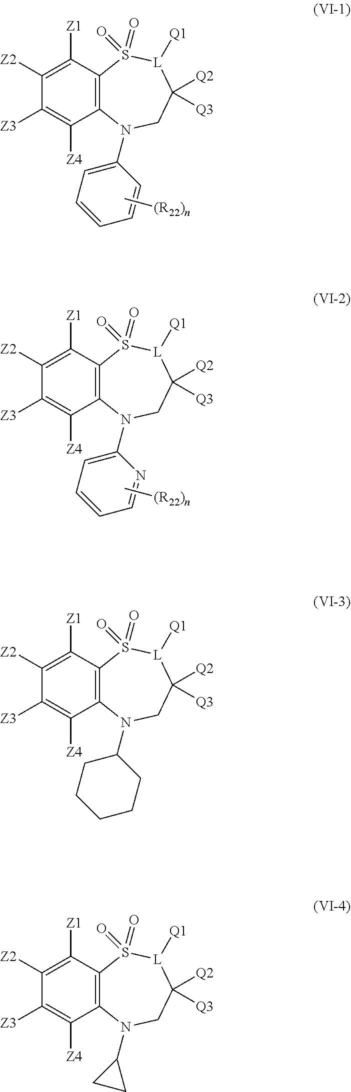

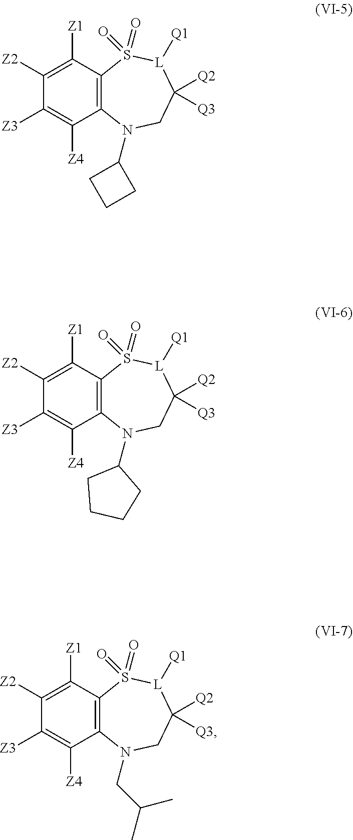

wherein n is 0, 1, 2, or 3; each R₂ is independently selected from: 1) halogen;
2) —CN;
3) —NO₂;
4) —OR₁₁;
5) —SR₁₁;
6) —NR₁₁R₁₂;
7) —OC(O)NR₁₁R₁₂
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
and Z1, Z2, Z3, Z4, L, Q1, Q2, Q3, Rn, and R₁₂ are as previously defined.

In one embodiment of the present invention, the compound of Formula (I) is represented by one of Formulae (VII-1) to (VII-4),

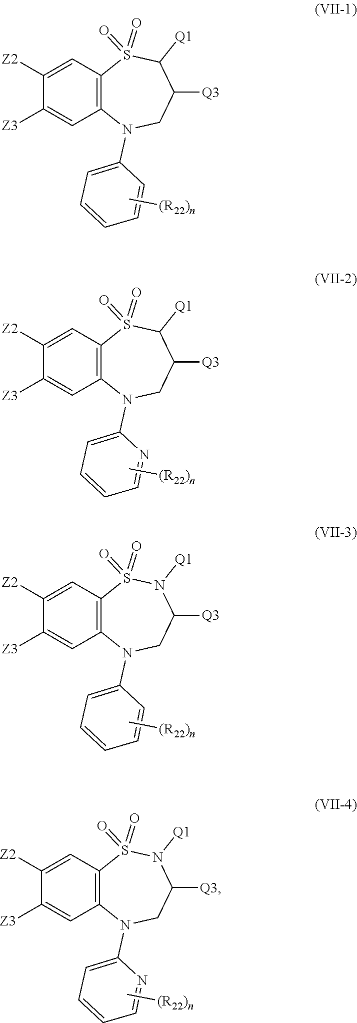

wherein Z2, Z3, Q1, Q3, n, and R₂₂ are as previously defined. Preferably n is 0 or1; when n is 1, R₂₂ is preferably —F, —C₁, —Br, —CN, —CH₃, —CF₃, —OH, or —OCH₃.

In one embodiment of the present invention, the compound of Formula (I) is represented by one of Formulae (VIII-1) to (VIII-8),

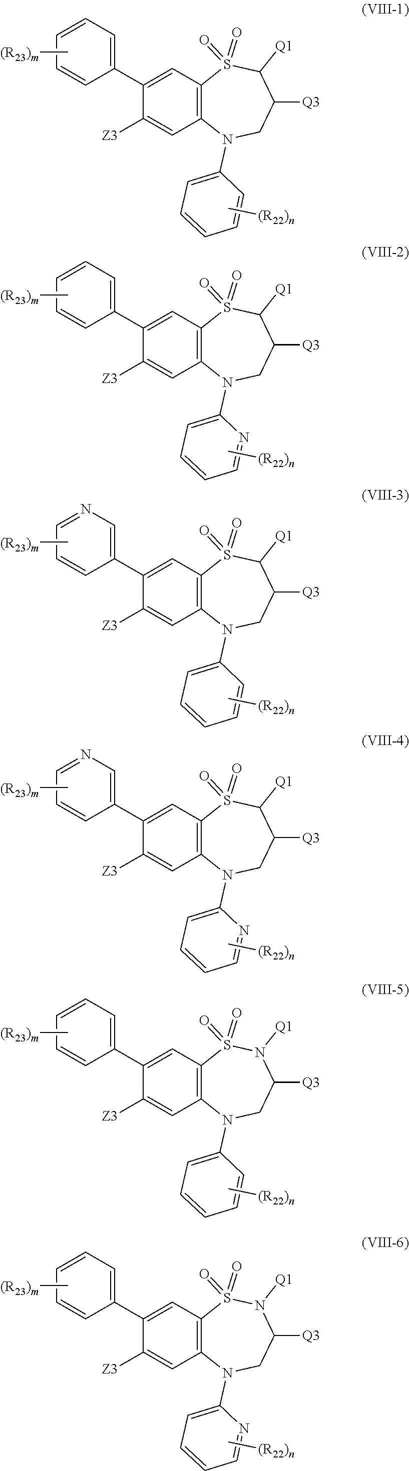

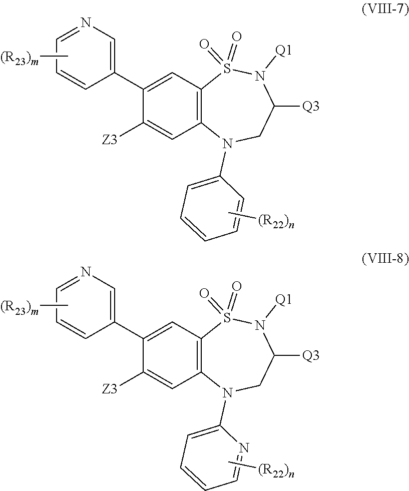

wherein m is 0, 1, 2, or 3; each R₂₃ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) —CO₂H;
9) —SO₃H;
10) —PO₃H₂;
11) —NHC(O)OR₁₁;
12) —NHS(O)₂R₁₁;
13) —NHC(O)R₁₁;
14) —SO₂NHC(O)R₁₁;
15) optionally substituted —C₁-C₆ alkyl;
16) optionally substituted —C₃-C₈ cycloalkyl;
17) optionally substituted 3- to 8-membered heterocycloalkyl;
18) optionally substituted aryl; and
19) optionally substituted heteroaryl;
   and Z3, Q1, Q3, n, and R₂₂ are as previously defined. Preferably m is 1 or 2 and each R₂₃ is independently —F, —Cl, —Br, —CN, —CH₃, —CF₃, —CO₂H, —OH, or —OCH₃. In certain embodiments, one R₂₃ is —CO₂H.

In one embodiment of the present invention, the compound of Formula (I) is represented by one of Formulae (VII-1) to (VII-4) or Formulae (VIII-1) to (VIII-8), wherein Z3 is —F, —Cl, —Br, —CN, —OCH₃, —SCH₃, —SCH₂CH₂, or —N(CH₃)₂; Q1 is hydrogen, or optionally substituted methyl; and Q3 is optionally substituted phenyl, optionally substituted benzyl, optionally substituted methyl, optionally substituted ethyl, optionally substituted t-butyl, optionally substituted isopropyl, optionally substituted isobutyl, optionally substituted neopentyl,

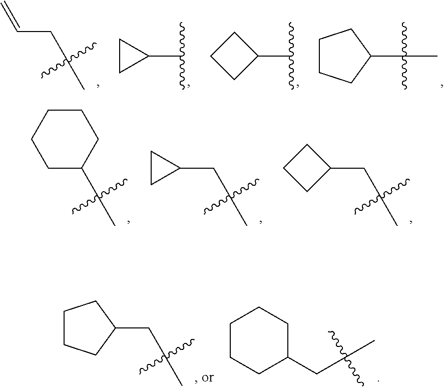

In one embodiment of the present invention, the compound of Formula (I) is represented by one of Formulae (IX-1) to (IX-3),

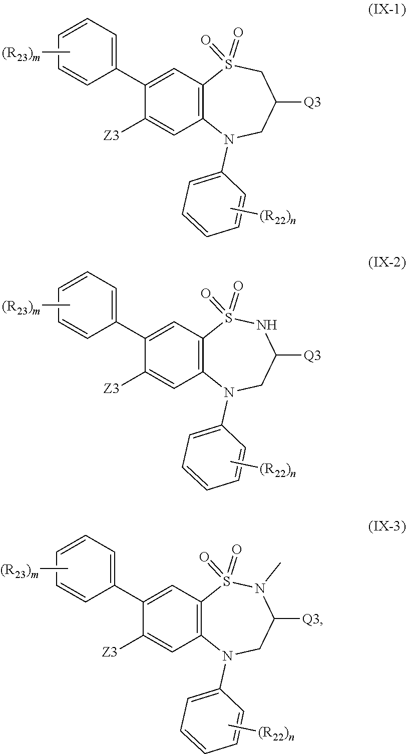

wherein Z3, Q3, n, m, R₂₂, and R₂₃ are as previously defined.

In one embodiment of the present invention, the compound of Formula (I) is represented by one of Formulae (IX-4) to (IX-6),

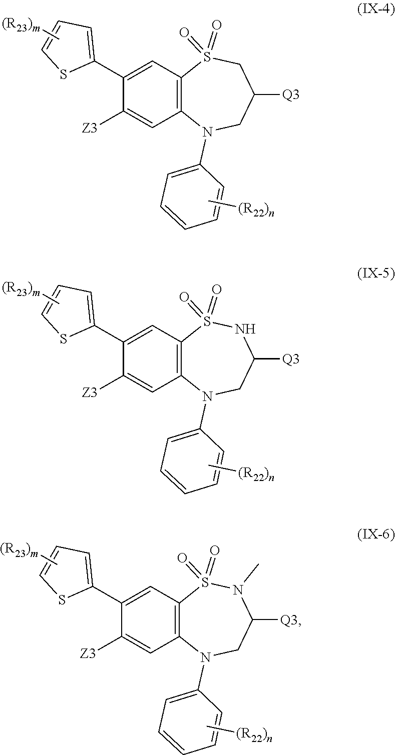

wherein Z3, Q3, n, m, R₂₂, and R₂₃ are as previously defined.

In one embodiment of the present invention, the compound of Formula (I) is represented by one of Formulae (IX-1) to (IX-6), wherein Z3 is —F, —Cl, —Br, —CN, —OCH₃, —SCH₃, —SCH₂CH₂, or —N(CH₃)₂; Q3 is optionally substituted phenyl, optionally substituted benzyl, optionally substituted methyl, optionally substituted ethyl, optionally substituted t-butyl, optionally substituted isopropyl, optionally substituted isobutyl, optionally substituted neopentyl,

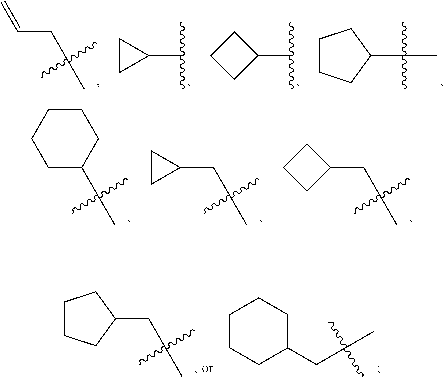

n is 0 or1; R₂₂ is —F, —Cl, —Br, —CN, —CH₃, —CF₃, —OH, or —OCH₃; m is 1 or 2; and R₂₃ is —F, —Cl, —Br, —CN, —CH₃, —CF₃,—CO₂H, —OH, or —OCH₃.

In one embodiment of the present invention, the compound of Formula (I) is represented by one of Formulae (X-1) to (X-6),

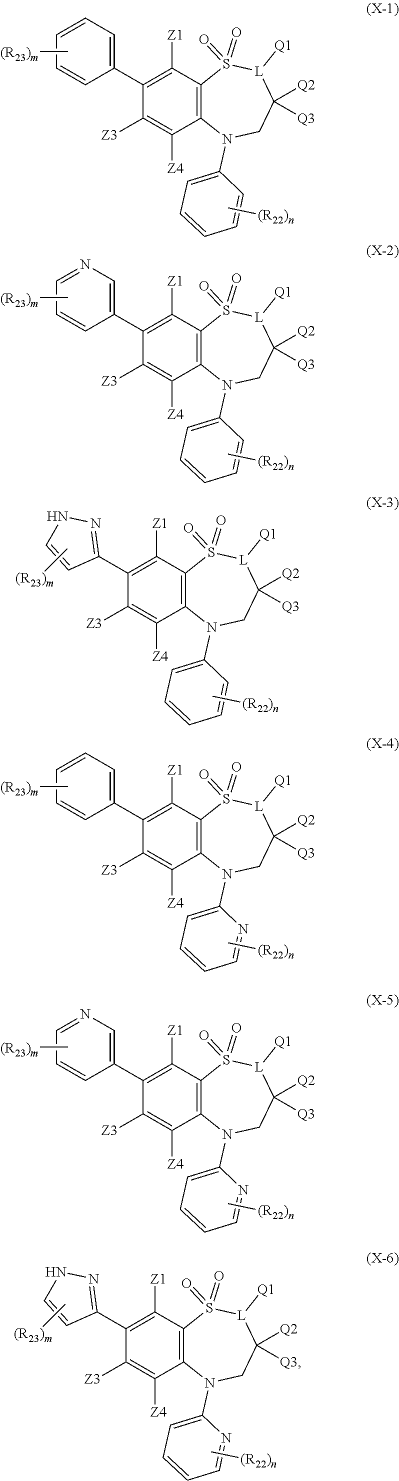

wherein Z1, Z3, Z4, L, Q1, Q2, Q3, n, m, R₂₂, and R₂₃ are as previously defined.

In one embodiment of the present invention, the compound of Formula (I) is represented by one of Formulae (XI-1) to (XI-2),

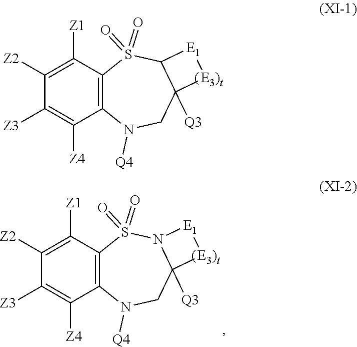

wherein t is 0, 1, 2, 3 or 4; E₁ is selected from —C(R₂₄)(R₂₅) — and —N(R₂₄) —; E₂ is —C(R₂₄)(R₂₅)—, E₃ at each occurrence is independently selected from —C(R₂₄)(R₂₅)—, —N(R₂₄)—, —O—, —S—, —S(O)—, and —S(O)₂—; R₂₄ and R₂₅ are each independently selected from the group consisting of hydrogen, halogen, —CN, —NO₂, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted 3- to 8-membered heterocyclic, optionally substituted aryl, and optionally substituted heteroaryl; and Z1, Z2, Z3, Z4, Q3, and Q4 are as previously defined. In certain embodiments, R₂₄ and R₂₅ are taken together with the carbon atom to which they attached to form an additional spiro ring. In certain embodiments, two adjacent R₂₄ groups are taken together with the atoms to which they are attached to form an olefinic double-bond or a fused ring. In certain embodiments, two remote R₂₄ groups are taken together with the atoms to which they are attached and any intervening atoms to form a bridging moiety.

In one embodiment of the present invention, the compound of Formula (I) is represented by one of Formulae (XII-1) to (XII-2),

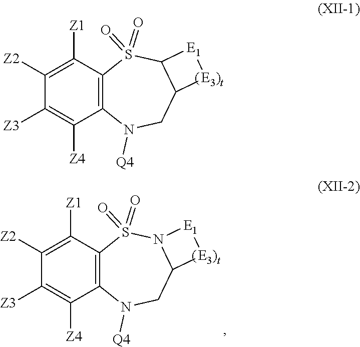

wherein Z1, Z2, Z3, Z4, Q4, t, E₁, E₂ and E₃ are as previously defined.

In one embodiment of the present invention, the compound of Formula (I) is represented by one of Formulae (XIII-1) to (XIII-2),

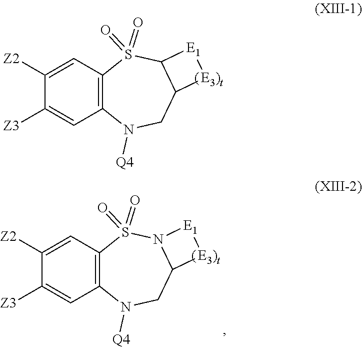

wherein Z2, Z3, Q4, t, E₁, E₂ and E₃ are as previously defined.

In one embodiment of the present invention, the compound of Formula (I) is represented by one of Formulae (XIV-1) to (XIV-2),

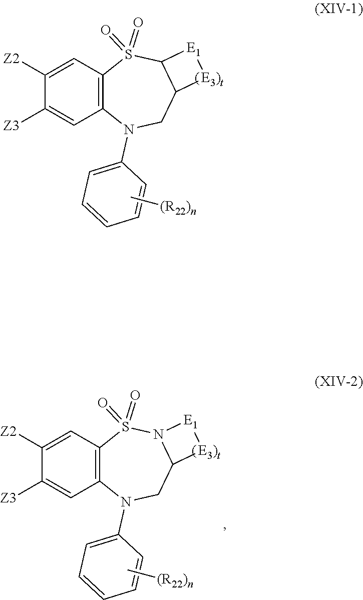

wherein n, R₂₂, Z2, Z3, t, E₁, E₂ and E₃ are as previously defined.

In one embodiment of the present invention, the compound of Formula (I) is represented by one of Formulae (XV-1) to (XV-2),

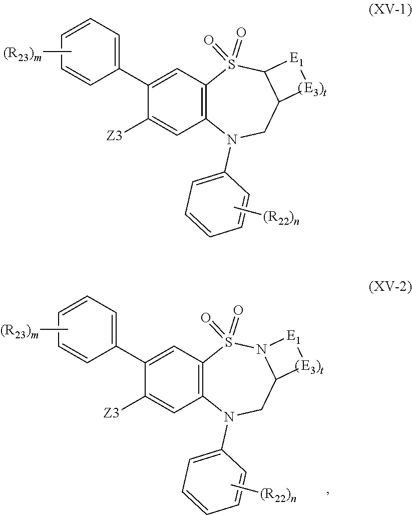

wherein n, R₂₂, m, R₂₃, Z3, t, E₁, E₂ and E₃ are as previously defined.

In one embodiment of the present invention, the compound of Formula (I) is represented by one of Formulae (XVI-1) to (XVI-4),

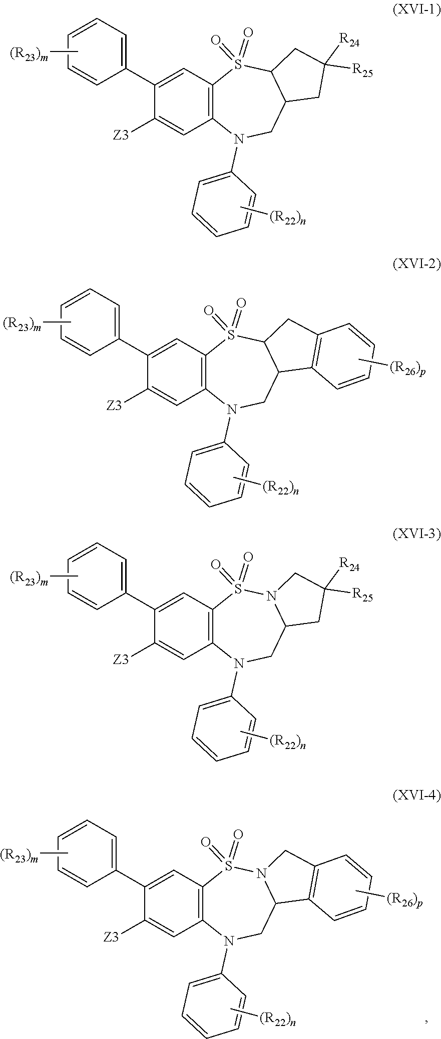

wherein p is 0, 1, 2, or 3; each R₂₆ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
   and n, R₂₂, m, R₂₃, Z3, R₂₄, and R₂₅ are as previously defined. Preferably p is 1 or 2 Each R₂₆ is preferably independently —F, —Cl, —Br, —CN, —CH₃, —CF₃,—CO₂H, —OH, or —OCH₃. In certain embodiments, R₂₄ and R₂₅ are taken together with the carbon atom to which they attached to form an additional spiro ring.

In one embodiment of the present invention, the compound of Formula (I) is represented by Formula (XVII),

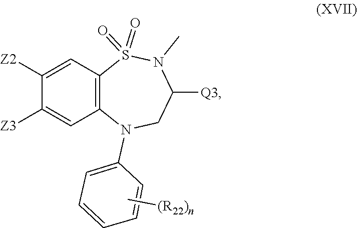

wherein Z2, Z3, Q3, n, and R₂₂ are as previously defined. Preferably n is 0 or1; wherein when n is 1, R₂₂ is preferably —F, —Cl, —Br, —CN, —CH₃, —CF₃, —OH, or —OCH₃.

In one embodiment of the present invention, the compound of Formula (I) is represented by Formula (XVIII-1) or Formula (XVIII-2),

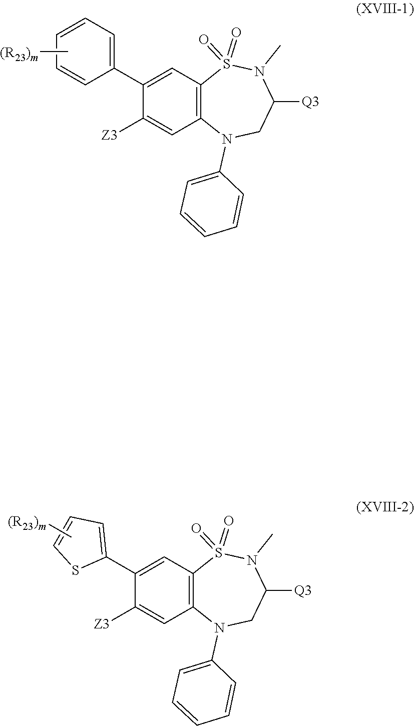

wherein Z3, Q3, m, and R₂₃ are as previously defined. Preferably m is 1 or 2 and R₂₃ is halogen, —CO₂H, —NR₁₂C(O)OR₁₁, NR₁₂C(O)R₁₁ or —NR₁₂S(O)₂R₁₁;

In one embodiment of the present invention, the compound of Formula (I) is represented by Formula (XIX-1) or Formula (XIX-2),

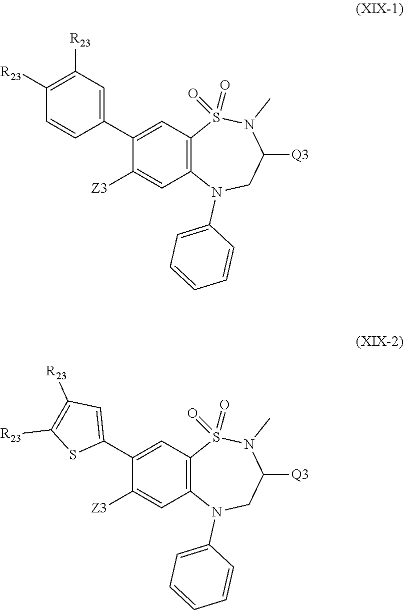

wherein Z3, Q3 and R₂₃ are as previously defined, and each R₂₃ can be same or different. Preferably each R₂₃ is independently halogen, —CO₂H, —NR₁₂C(O)OR₁₁, NR₁₂C(O)R₁₁ or —NR₁₂S(O)₂R₁₁.

In one embodiment of the present invention, the compound of Formula (I) is represented by one of Formulae (XX-1) to (XX-4),

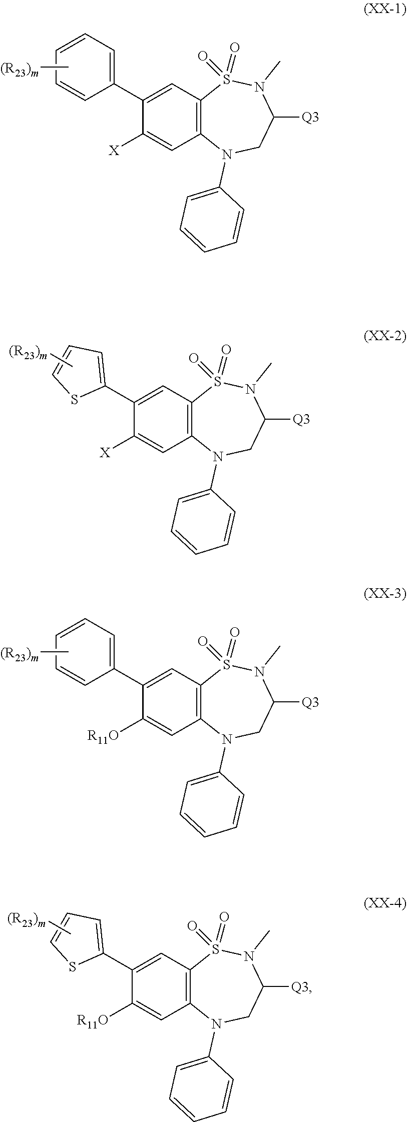

wherein X is halogen; Q3, R₁₁, m and R₂₃ are as previously defined, and each R₂₃ can be same or different. Preferably m is 1 or 2 and each R₂₃ is independently halogen, —CO₂H, —NR₁₂C(O)OR₁₁, NR₁₂C(O)R₁₁ or —NR₁₂S(O)₂R₁₁.

In one embodiment of the present invention, the compound of Formula (I) is represented by one of Formulae (XXI-1) to (XXI-4),

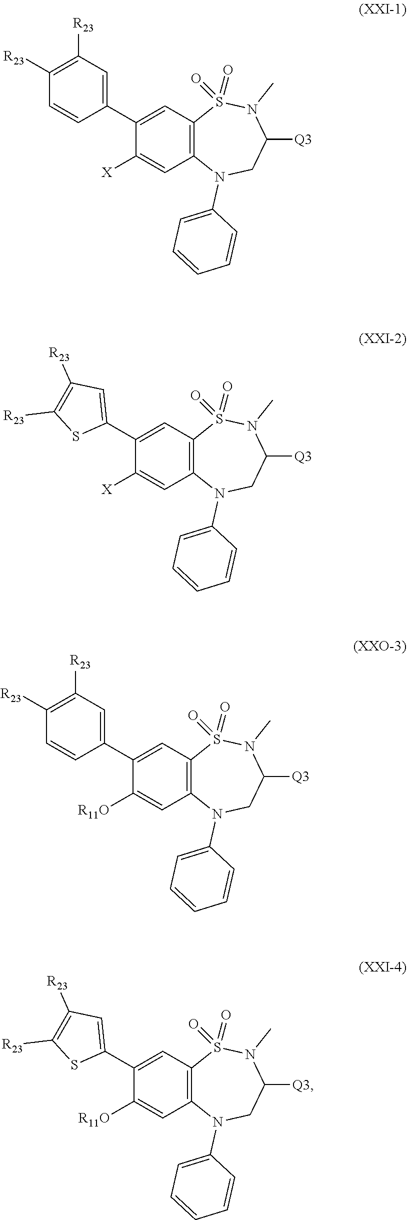

wherein X, Q3, R₁₁, and R₂₃ are as previously defined, and each R₂₃ can be same or different. Preferably each R₂₃ is independently halogen, —CO₂H, —NR₁₂C(O)OR₁₁, NR₁₂C(O)R₁₁ or —NR₁₂S(O)₂R₁₁.

In one embodiment of the present invention, the compound of Formula (I) is represented by one of Formulae (XVII), (XIX-1), (XIX-2), (XX-1) to (XX-4), and (XXI-1) to (XXI-4), wherein Q3 is optionally substituted phenyl, optionally substituted benzyl, optionally substituted methyl, optionally substituted ethyl, optionally substituted t-butyl, optionally substituted isopropyl, optionally substituted isobutyl, optionally substituted neopentyl,

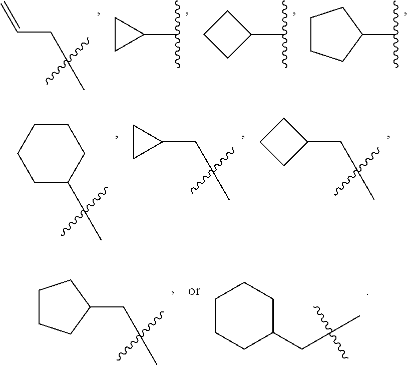

It will be appreciated that the description of the present invention herein should be construed in congruity with the laws and principles of chemical bonding. In some instances, it may be necessary to remove a hydrogen atom in order to accommodate a substituent at any given location.

It will be yet appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

In one embodiment, the compounds described herein are suitable for monotherapy and are effective against natural or native HBV and/or HDV strains and against HBV and/or HDV strains resistant to currently known drugs. In another embodiment, the compounds described herein are suitable for use in combination therapy.

In another embodiment, the additional therapeutic agent is selected from a core inhibitor, which includes GLS4, GLS4JHS, JNJ-379, ABI-H0731, ABI-H2158, AB-423, AB-506, WX-066, and QL-OA6A; immune modulator or immune stimulator therapies, which includes T-cell response activator AIC649 and biological agents belonging to the interferon class, such as interferon alpha 2a or 2b or modified interferons such as pegylated interferon, alpha 2a, alpha 2b, lamda; or STING (stimulator of interferon genes) modulator; or TLR modulators such as TLR-7 agonists, TLR-8 agonists or TLR-9 agonists; or therapeutic vaccines to stimulate an HBV-specific immune response such as virus-like particles composed of HBcAg and HBsAg, immune complexes of HBsAg and HBsAb, or recombinant proteins comprising HBx, HBsAg and HBcAg in the context of a yeast vector; or immunity activator such as SB-9200 of certain cellular viral RNA sensors such as RIG-I, NOD2, and MDA5 protein, or RNA interence (RNAi) or small interfering RNA (siRNA) such as ARC-520, ARC-521, ARB-1467, and ALN-HBV RNAi, or antiviral agents that block viral entry or maturation or target the HBV polymerase such as nucleoside or nucleotide or non-nucleos(t)ide polymerase inhibitors, and agents of distinct or unknown mechanism including agents that disrupt the function of other essential viral protein(s) or host proteins required for HBV replication or persistence such as REP 2139, RG7834, and AB-452. In an embodiment of the combination therapy, the reverse transcriptase inhibitor is at least one of Zidovudine, Didanosine, Zalcitabine, ddA, Stavudine, Lamivudine, Aba-cavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In another embodiment of the combination therapy, the TLR-7 agonist is selected from the group consisting of SM360320 (12-benzyl-8-hydroxy-2-(2-methoxy-ethoxy)adenine), AZD 8848 (methyl [3-({[3-(6-amino-2-butoxy-8-oxo-7,8-dihydro-9H-purin-12-yl)propyl][3-(4-morpholinyl)propyl]amino)methyl)phenyl]acetate), GS-9620 (4-Amino-2-butoxy-8-[3-(2-pyrrolidinylmethyl)benzyl]-7,8-dihydro-6(5H)-pteridinone), AL-034 (TQ-A3334), and RO6864018.

In another embodiment of the combination therapy, the TLR-8 agonist is GS-9688.

In an embodiment of these combination therapies, the compound and the additional therapeutic agent are co-formulated. In another embodiment, the compound and the additional therapeutic agent are co-administered.

In another embodiment of the combination therapy, administering the compound of the invention allows for administering of the additional therapeutic agent at a lower dose or frequency as compared to the administering of the at least one additional therapeutic agent alone that is required to achieve similar results in prophylactically treating an HBV infection in an individual in need thereof.

In another embodiment of the combination therapy, before administering the therapeutically effective amount of the compound of the invention, the individual is known to be refractory to a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

In still another embodiment of the method, administering the compound of the invention reduces viral load in the individual to a greater extent compared to the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

In another embodiment, administering of the compound of the invention causes a lower incidence of viral mutation and/or viral resistance than the administering of a compound selected from the group consisting of a HBV polymerase inhibitor, interferon, viral entry inhibitor, viral maturation inhibitor, distinct capsid assembly modulator, antiviral compounds of distinct or unknown mechanism, and combination thereof.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “alkyl” as used herein, refers to saturated, straight- or branched-chain hydrocarbon radicals. “C₁.C₄ alkyl,” “C₁-C₆ alkyl,” “C₁-C₈ alkyl,” “C₁.C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of C₁-C₈ alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of C₃-C₁₂ cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of C₃-C₁₂ cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” is a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups.Examples of alkoxy groups includes, but are not limited to, methoxy, ethoxy, n-propoxy, 2-propoxy (isopropoxy) and the higher homologs and isomers. Preferred alkoxy are C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic groups may be further substituted. Heteroaryl or Heterocyclic groups can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, CO₂—C₃-C₁₂-cycloalkyl, —CO₂— aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH— heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂—C₁-C₁₂-alkyl, —NHCO₂—C₂-C₈-alkenyl, —NHCO₂— C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH— heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(0) —C₂-C₈-alkenyl, —S(0) —C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH— C₂-C₈-alkynyl, —SO₂NH—C₃-C₁₂-cycloalkyl, —SO₂NH-aryl, —SO₂NH-heteroaryl, —SO₂NH— heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably C₁ and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

Combination and Alternation Therapy

It has been recognized that drug-resistant variants of HIV, HBV and HCV can emerge after prolonged treatment with an antiviral agent. Drug resistance most typically occurs by mutation of a gene that encodes for a protein such as an enzyme used in viral replication, and most typically in the case of HIV, reverse transcriptase, protease, or DNA polymerase, and in the case of HBV, DNA polymerase, or in the case of HCV, RNA polymerase, protease, or helicase. Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. The compounds can be used for combination are selected from the group consisting of a HBV polymerase inhibitor, interferon, TLR modulators such as TLR-7 agonists or TLR-9 agonists, therapeutic vaccines, immune activator of certain cellular viral RNA sensors, viral entry inhibitor, viral maturation inhibitor, distinct capsid assembly modulator, antiviral compounds of distinct or unknown mechanism, and combination thereof. Alternatively, the pharmacokinetics, biodistribution, or other parameter of the drug can be altered by such combination or alternation therapy. In general, combination therapy is typically preferred over alternation therapy because it induces multiple simultaneous stresses on the virus.

Preferred compounds for combination or alternation therapy for the treatment of HBV include 3TC, FTC, L-FMAU, interferon, adefovir dipivoxil, entecavir, telbivudine (L-dT), valtorcitabine (3′-valinyl L-dC), β-D-dioxolanyl-guanine (DXG), β-D-dioxolanyl-2,6-diaminopurine (DAPD), and β-D-dioxolanyl-6-chloropurine (ACP), famciclovir, penciclovir, lobucavir, ganciclovir, and ribavirin.

Although the invention has been described with respect to various preferred embodiments, it is not intended to be limited thereto, but rather those skilled in the art will recognize that variations and modifications may be made therein which are within the spirit of the invention and the scope of the appended claims.

Antiviral Activity

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantadine). The compositions according to the invention may also be used in combination with gene replacement therapy.

### Abbreviations

Abbreviations which may be used in the descriptions of the scheme and the examples that follow are: Ac for acetyl; AcOH for acetic acid; Boc₂O for di-tert-butyl-dicarbonate; Boc for t-butoxycarbonyl; Bz for benzoyl; Bn for benzyl; t-BuOK for potassium tert-butoxide; Brine for sodium chloride solution in water; CDI for carbonyldiimidazole; DCM or CH₂C₂ for dichloromethane; CH₃ for methyl; CH₃CN for acetonitrile; Cs₂CO₃ for cesium carbonate; CuCl for copper (I) chloride; CuI for copper (I) iodide; dba for dibenzylidene acetone; DBU for 1,8-diazabicyclo[5.4.0]-undec-7-ene; DEAD for diethylazodicarboxylate; DIAD for diisopropyl azodicarboxylate; DIPEA or (i-Pr)₂EtN for N,N,-diisopropylethyl amine; DMP or Dess-Martin periodinane for 1,1,2-tris(acetyloxy)-1,2-dihydro-1,2-benziodoxol-3-(1H)-one; DMAP for 4-dimethylamino-pyridine; DME for 1,2-dimethoxyethane; DMF for N,N-dimethylformamide; DMSO for dimethyl sulfoxide; EtOAc for ethyl acetate; EtOH for ethanol; Et₂O for diethyl ether; HATU for O-(7-azabenzotriazol-2-yl)-N,N,N′,N′,-tetramethyluronium Hexafluoro-phosphate; HCl for hydrogen chloride; K₂CO₃ for potassium carbonate; n-BuLi for n-butyl lithium; DDQ for 2,3-dichloro-5,6-dicyano-1,4-benzoquinone; LDA for lithium diisopropylamide; LiTMP for lithium 2,2,6,6-tetramethyl-piperidinate; MeOH for methanol; Mg for magnesium; MOM for methoxymethyl; Ms for mesyl or —SO₂—CH₃; NaHMDS for sodium bis(trimethylsilyl)amide; NaCl for sodium chloride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfite or sodium hydrogen sulfite; Na₂S₂O₃ for sodium thiosulfate; NH₂NH₂ for hydrazine; NH₄C₁ for ammonium chloride; Ni for nickel; OH for hydroxyl; OsO₄ for osmium tetroxide; OTf for triflate; PPA for polyphophoric acid; PTSA forp-toluenesulfonic acid; PPTS for pyridinium p-toluenesulfonate; TBAF for tetrabutylammonium fluoride; TEA or Et₃N for triethylamine; TES for triethylsilyl; TESC1 for triethylsilyl chloride; TESOTf for triethylsilyl trifluoromethanesulfonate; TFA for trifluoroacetic acid; THE for tetrahydrofuran; TMEDA for N,N,N′,N′-tetramethylethylene-diamine; TPP or PPh₃ for triphenyl-phosphine; Tos or Ts for tosyl or —SO₂—C₆H₄CH₃; Ts₂O for tolylsulfonic anhydride or tosyl-anhydride; TsOH for p-tolylsulfonic acid; Pd for palladium; Ph for phenyl; Pd₂(dba)₃ for tris(diben-zylideneacetone) dipalladium (0); Pd(PPh₃)₄ for tetrakis(triphenylphosphine)-palladium (0); PdCl₂(PPh₃)₂ for trans-dichlorobis-(triphenylphosphine)palladium (II); Pt for platinum; Rh for rhodium; rt for room temperature; Ru for ruthenium; TBS for tert-butyl dimethylsilyl; TMS for trimethylsilyl; or TMSCI for trimethylsilyl chloride.

Synthetic Methods

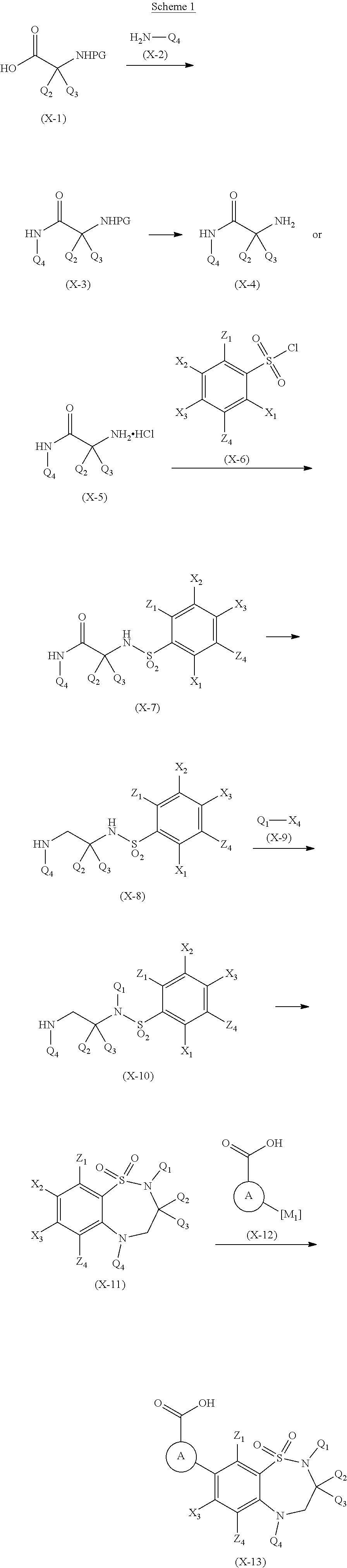

Scheme 1 illustrates a general method to prepare the compound of formula (X-13) from an optically pure or racemic amino acid derivative (X-1), wherein, Q1, Q2, Q3, Q4, and Z1, and Z4 are as previously defined, X1, X2, X3, and X4 are suitably chosen halogen atoms or pseudo-halogen groups. The group PG represents a viable protecting group. Amine protecting groups are described in T. H. Greene and P. G. M. Wuts, Protective Groups in Organic Synthesis, 3^{rd} edition, John Wiley & Sons, New York (1999). A represents an optionally substituted aromatic, heteroaromatic, optionally substituted C₃-C₈ cycloalkyl, or optionally substituted 3- to 12-membered heterocycloalkyl. The carboxylic acid functional group of (X-1) can be condensed with the amine (X-2) under commonly employed amide coupling conditions (HATU, EDC, DCC, etc) or following activation by a suitable chloroformate (e,g, isobutyl chloroformate) in the presence of base to form compound (X-3). The protecting group (PG) can be removed to liberate the free base (X-4) or a corresponding ammonium salt, depicted here as hydrochloride salt (X-5), depending upon the identity of the protecting group and the conditions employed for its removal. Upon removal of PG, compound (X-4) or (X-5) is reacted with compound (X-6) in the presence of base to afford (X-7). The carbonyl functional group of compound (X-7) is reduced using a suitable hydride reagent (BH₃ THF, BH₃·SMe₂, AlH₃, LiAlH₄, etc) to afford compound (X-8). Compound (X-8) is reacted with compound (X-9) with loss of X4 to afford the tertiary sulfonamide (X-10). Compound (X-10) is cyclized via a nucleophilic aromatic substitution reaction (SNAr) or in the presence of base and a metal catalyst via a catalytic C—N coupling reaction with concomitant loss of X1 to afford (X-11). Product (X-11) is reacted with compound (X-12), wherein [M¹] represents B(OH)₂, BF₃K, B(OR)₂, SnR₃, or ZnX, under the action of a metal catalyst and base to furnish compound (X-13).

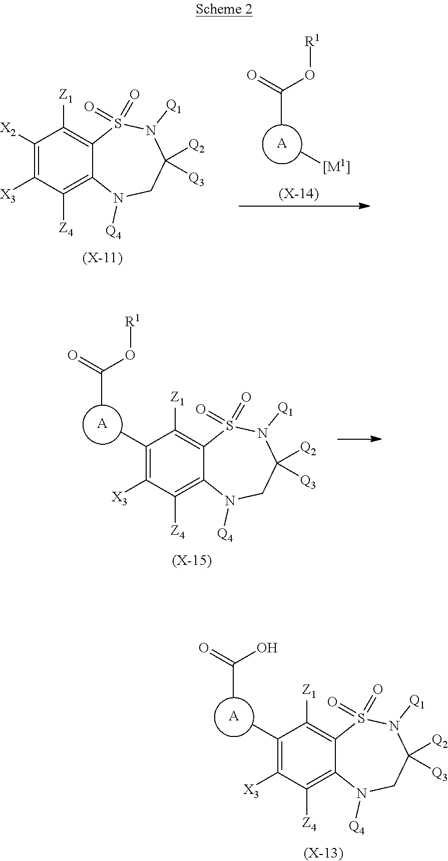

Scheme 2 provides an alternative method to prepare the compound of formula (X-13) beginning from (X-11), wherein Q1, Q2, Q3, Q4, Z1, Z4, X2, X3, A, and [M¹] are as previously described. R1 is an alkyl group. Compound (X-11) can be reacted compound (X-14) under the action of a metal catalyst and base to furnish compound (X-15). In some examples, the coupling between (X-11) and (X-14) to form (X-15) is promoted by light in the presence of an additional metal catalyst and a tertiary amine such as morpholine. Hydrolysis of (X-15) furnishes compound (X-13).

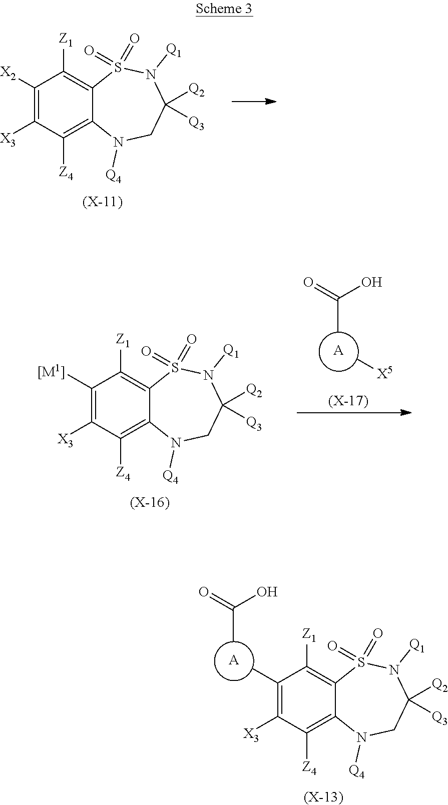

Scheme 3 provides an additional method to prepare the compound of formula (X-13) beginning from (X-11). Compound (X-11) is transformed to compound (X-16) in the presence of a metal containing catalyst and an appropriate [M¹] containing reagent (e.g. B₂Pin₂) to afford (X-16). Compound (X-16) is then reacted with (X-17), wherein X5 is a suitable halogen atom or pseudo-halogen group, in the presence of a metal catalyst and base to afford compound (X-13).

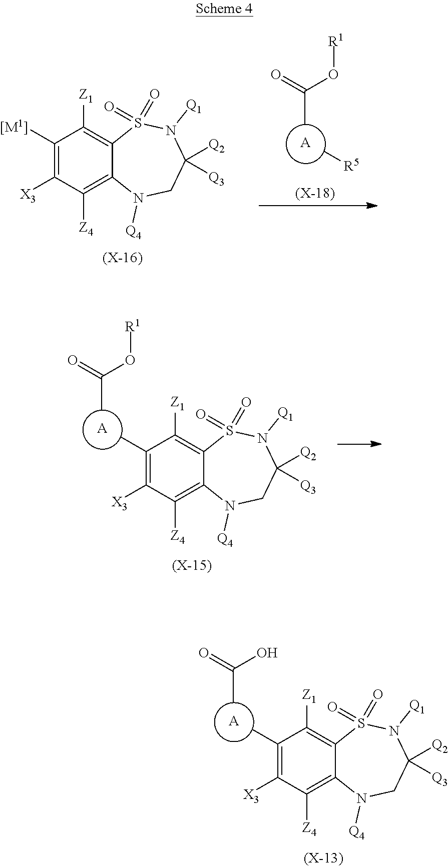

As illustrated in Scheme 4, in some examples, a compound of formula (X-16) is reacted with compound (X-18) to furnish compound (X-15). As previously described, hydrolysis of compound (X-15) provides compound (X-13).

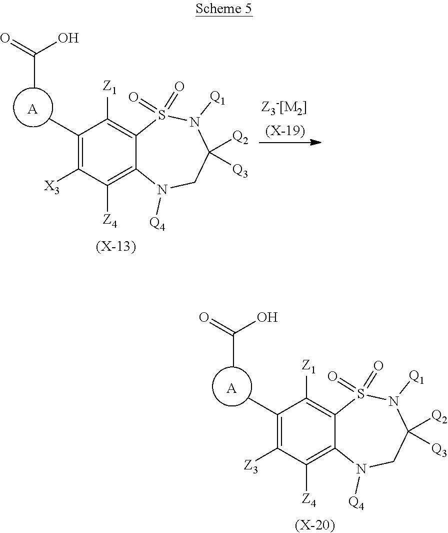

Scheme 5 illustrates a method for the preparation of compound (X-20), wherein Q1, Q2, Q3, Q4, Z1, Z4, Z3, and A are as previously described. In some examples, [M²] represents H, and Z3-[M²] defines a protic pro-nucleophile that reacts with (X-13) with concomitant loss of X3 in the presence of base with or without a metal containing catalyst. Alternatively, [M²] represents B(OH)₂, BF₃K, B(OR)₂, ZnX, SnR₃, or Na (e.g. Z3=CN and Z3-1[M²] is NaCN) and compound (X-13) and (X-19) react in the presence of a metal containing catalyst with or without base to form compound (X-20).

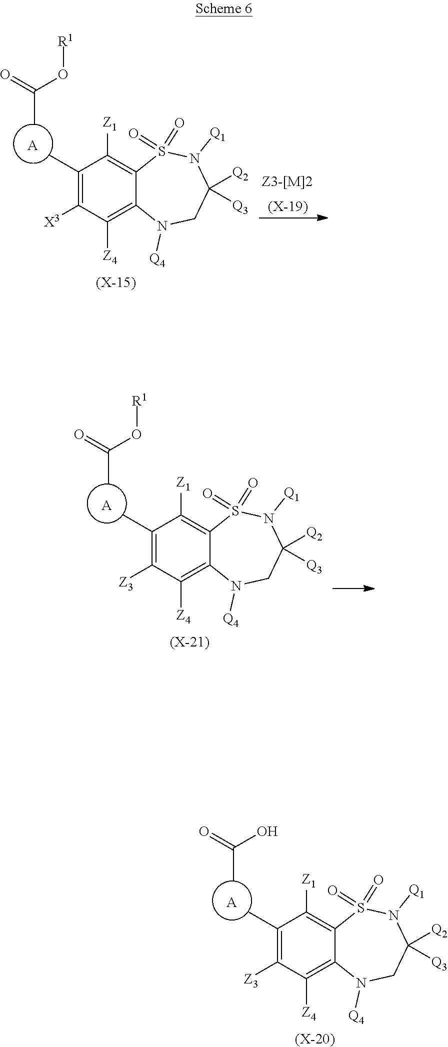

Scheme 6 illustrates a method for the preparation of compound (X-20), wherein Q1, Q2, Q3, Q4, Z1, Z4, Z3, and A are as previously described. R1 is an alkyl group. In some examples, [M²] represents H, and Z3-[M²] defines a protic pro-nucleophile that reacts with (X-15) with concomitant loss of X3 in the presence of base with or without a metal containing catalyst. Alternatively, [M²] represents B(OH)₂, BF₃K, B(OR)₂, ZnX, SnR₃, or Na (e.g. Z3=CN and Z3-[M²] is NaCN) and compound (X-13) and (X-21) react in the presence of a metal containing catalyst with or without base to form compound (X-20).

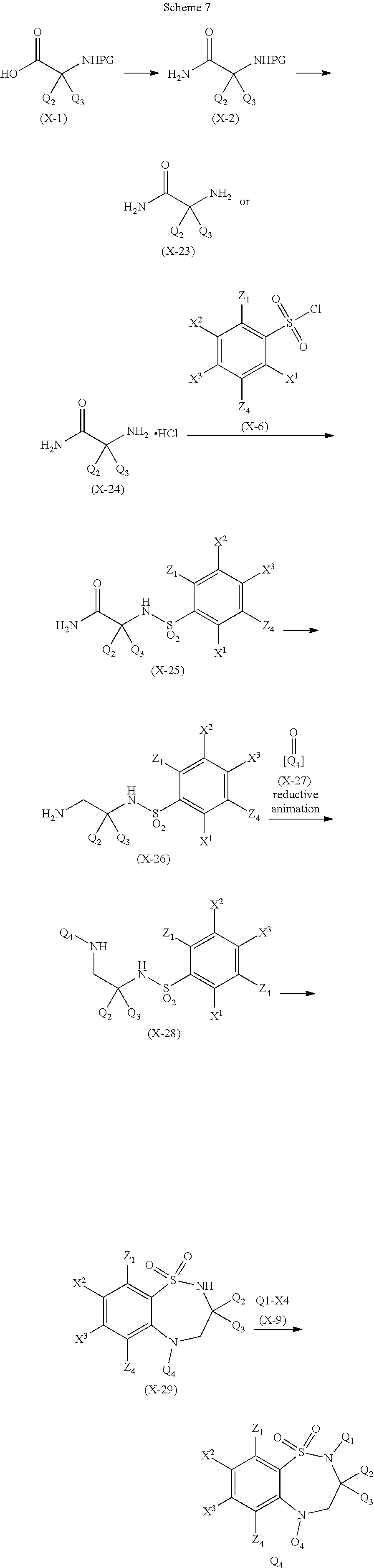

Scheme 7 illustrates an alternative method for the preparation of compound (X-11), wherein Q1, Q2, Q3, Q4, Z1, Z4, X1, X2, and X3, X4 are as previously defined. The carboxylic acid functional group of (X-1) can be condensed with an ammonia source (NH₃, NH₄₀H, or NH₃C₁) under commonly employed amide coupling conditions (HATU, EDC, DCC, etc) or following activation by a suitable chloroformate (e,g, isobutyl chloroformate) in the presence of base to form compound (X-22). The protecting group (PG) can be removed to liberate the free base (X-23) or a corresponding ammonium salt, depicted here as hydrochloride salt (X-24), depending upon the identity of the protecting group and the conditions employed for its removal. Upon removal of PG, compound (X-23) or (X-24) is reacted with compound (X-6) in the presence of base to afford (X-25). The carbonyl functional group of compound (X-25) is reduced using a suitable hydride reagent (BH₃ THF, BH₃ SMe₂, AlH₃, LiAlH₄, etc) to afford compound (X-26). Compound (X-26) reacts with ketone or aldehyde (X-27) in the presence of an appropriate reducing reagent (NaHB(OAc)₃ or NaBH₃CN) via a reductive amination process to afford compound (X-28). Compound (X-28) is cyclized via a nucleophilic aromatic substitution reaction in the presence of base (SNAr) or in the presence of base and a metal catalyst via a catalytic C—N coupling reaction with concomitant loss of X1 to afford (X-29). Finally, compound (X-29) undergoes an alkylation reaction with (X-9) to afford (X-11).

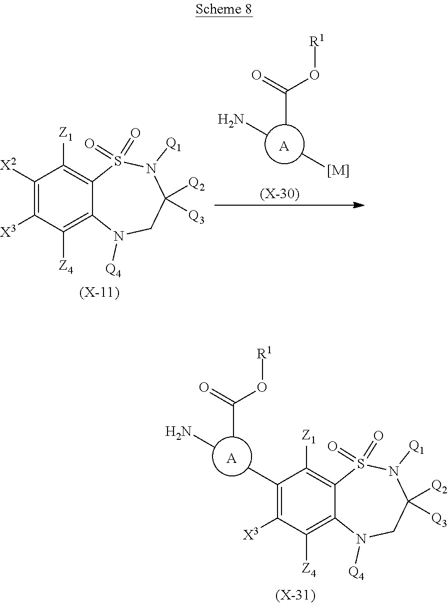

Scheme 8 illustrates a method for the preparation of compound (X-31), wherein Q1, Q2, Q3, Q4, Z1, Z4, X3, and ring A are as previously described, and ring A contains a primary amine. Compound (X-11) is reacted with (X-30) under the action of a metal catalyst and base to furnish compound (X-31).

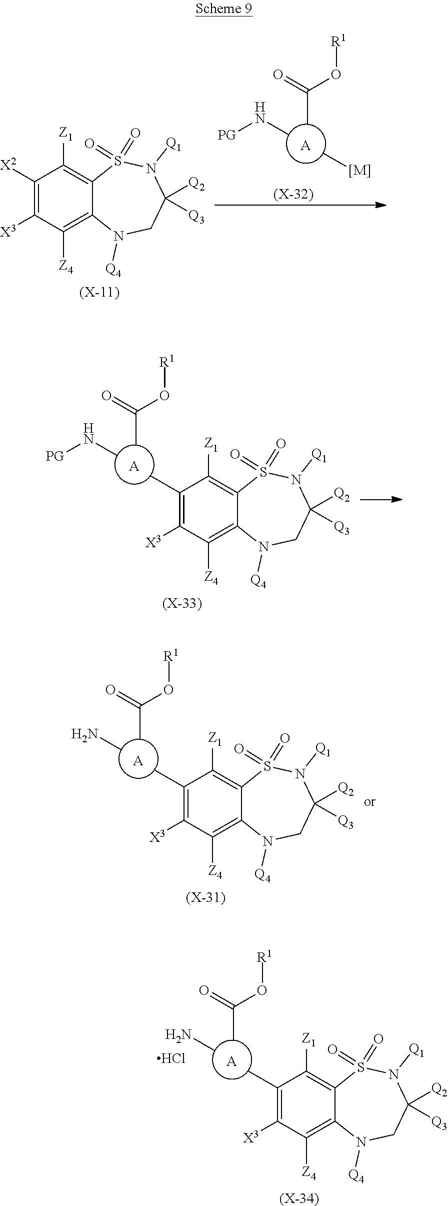

Scheme 9 illustrates a method for the preparation of (X-34) and an alternative method for the preparation of (X-31). First, (X-11) is reacted with (X-32), in which the amine functionality bears an appropriate protecting group PG, in the presence of a metal containing catalyst and base to afford (X-33). Next, (X-33) is subjected to protecting group removal to afford (X-31) or a corresponding ammonium salt, depicted herein as hydrochloride salt (X-34), depending on the identity of the protecting group and the conditions employed.

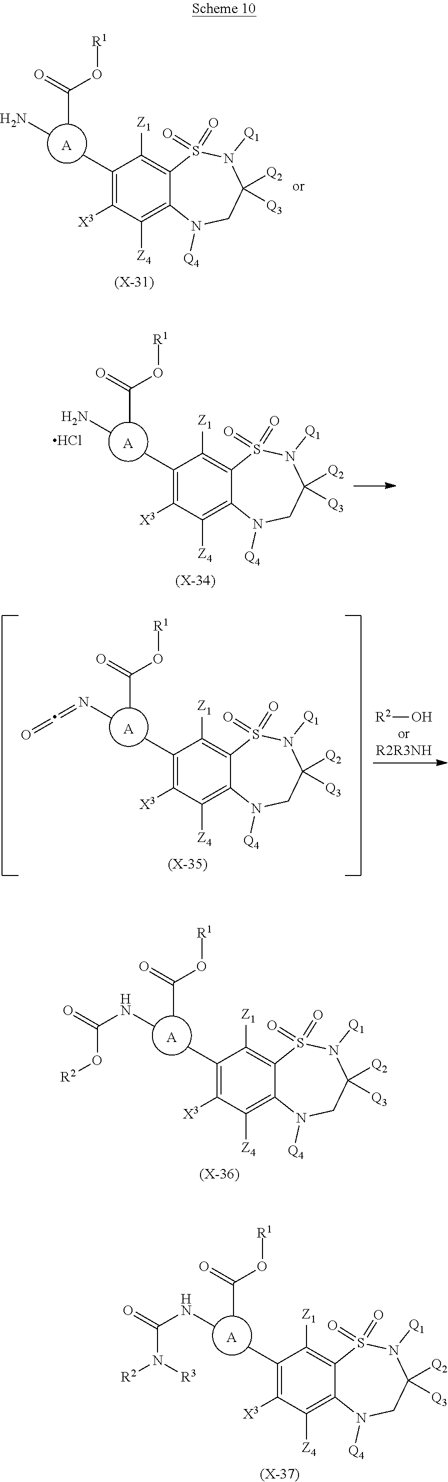

Scheme 10 illustrates the formation of a compound of formula (X-36) and (X-37) starting from compound (X-31) or (X-34). First, compound (X-31) or (X-34) is transformed to the corresponding isocyanate (X-35) via reaction with an appropriate C₁ electrophile such as phosgene or triphosgene in the presence of base. Compound (X-35) is then reacted in situ with an alcohol or amine to furnish compounds (X-36) and (X-37), respectively.

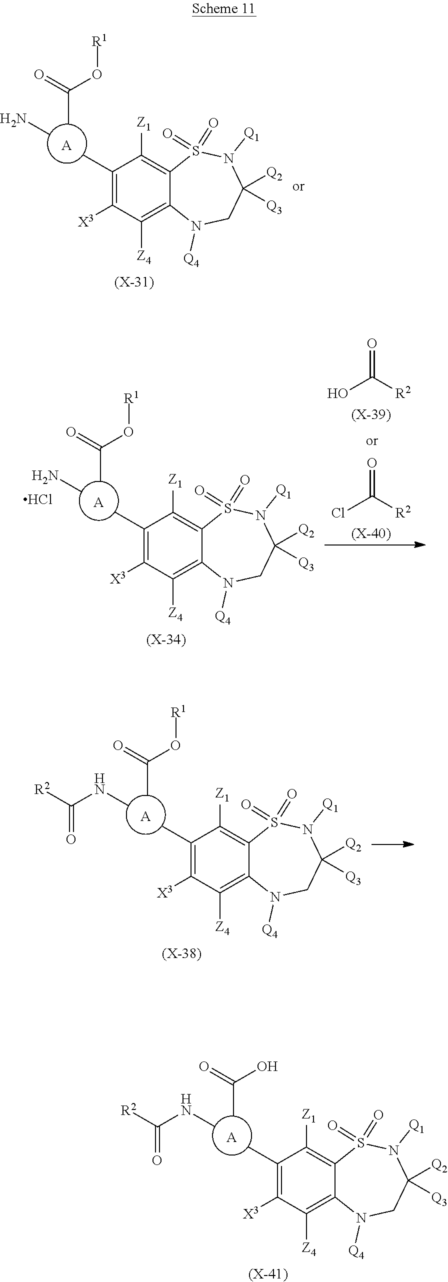

Scheme 11 illustrates a method for the preparation of amide (X-41) starting from (X-31) or (X-34). Compound (X-31) or a corresponding ammonium salt, depicted here as the hydrochloride salt (X-34), is reacted with a carboxylic acid (X-39) under commonly employed amide coupling conditions (HATU, EDC, DCC, etc) or with an acid chloride (X-40) in the presence of base to afford compound (X-38). Hydrolysis of (X-38) affords (X-41).

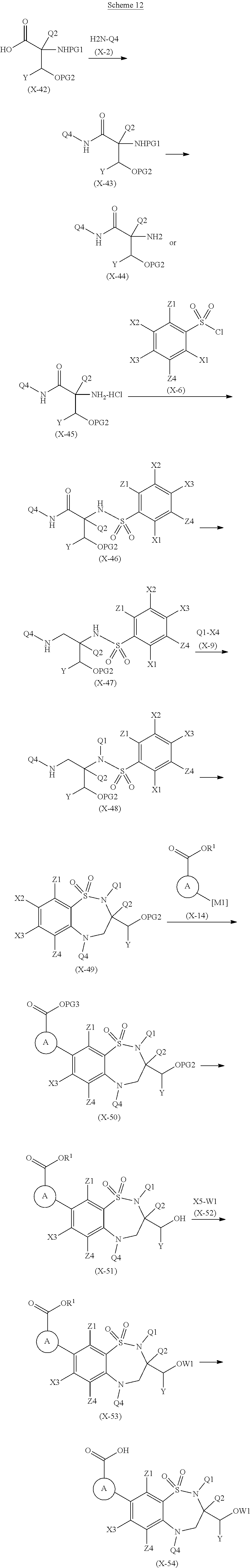

In some examples amino acid derivative (X-1) is a serine or threonine derivative (X-42, Y=H or Me) and scheme 12 illustrates a general method to prepare the compound of formula (X-54), wherein, Q1, Q2, Q3, Q4, and Z1, and Z4 are as previously defined, X1, X2, X3, X4, and X5 are suitably chosen halogen atoms or pseudo-halogen groups. PG1 and PG2 represent viable protecting groups. Wi is an alkyl group. Amine, alcohol and carboxylic acid protecting groups are described in T. H. Greene and P. G. M. Wuts, Protective Groups in Organic Synthesis, 3^{rd} edition, John Wiley & Sons, New York (1999). A represents an optionally substituted aromatic, heteroaromatic, optionally substituted C₃-C₈ cycloalkyl, or optionally substituted 3- to 12-membered heterocycloalkyl. The carboxylic acid functional group of (X-42) can be condensed with the amine (X-2) under commonly employed amide coupling conditions (HATU, EDC, DCC, etc) or following activation by a suitable chloroformate (e.g. isobutyl chloroformate) in the presence of base to form compound (X-43). The protecting group PG1 can be removed to liberate the free base (X-44) or a corresponding ammonium salt, depicted herein as the hydrochloride salt (X-45), dependent upon the identity of the protecting group and the conditions employed for its removal. Upon removal of PG1, compound (X-44) or (X-45) is reacted with compound (X-6) in the presence of base to afford (X-46). The carbonyl functional group of compound (X-46) is reduced using a suitable hydride reagent (BH₃ THF, BH₃ SMe₂, AlH₃, LiAlH₄, etc) to afford compound (X-47). Compound (X-47) is reacted with compound (X-9) with loss of X4 to afford the tertiary sulfonamide (X-48). Compound (X-48) is cyclized via a nucleophilic aromatic substitution reaction (SNAr) or in the presence of base and a metal catalyst via a catalytic C—N coupling reaction with concomitant loss of X1 to afford (X-49). Product (X-49) is reacted with compound (X-14), wherein [M¹]represents B(OH)₂, BF₃K, B(OR)₂, SnR₃, or ZnX, under the action of a metal containing catalyst and base to furnish compound (X-50). Protecting group PG2 is removed, giving compound (X-51). Compound (X-51) is treated with compound (X-52) with loss of X5 to afford ether (X-53). Hydrolysis then affords (X-54).

The compounds and processes of the present invention will be better understood in connection with the following synthetic schemes that illustrate the methods by which the compounds of the invention may be prepared. These schemes are of illustrative purpose and are not meant to limit the scope of the invention. Equivalent, similar, or suitable solvents, reagents or reaction conditions may be substituted for those particular solvents, reagents, or reaction conditions described herein without departing from the general scope of the method of synthesis.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### Examples

The compounds and processes of the present invention will be better understood in connection with the following examples, which are intended as an illustration only and not limiting of the scope of the invention. Starting materials were either available from a commercial vendor or produced by methods well known to those skilled in the art.

General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C18 column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]⁺ refers to mono-isotopic molecular weights.

NMR spectra were run on a Bruker 400 MHz spectrometer. Spectra were measured at 298K and referenced using the solvent peak. Chemical shifts for ¹H NMR are reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified.

Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Ex.1: Synthesis of 2-fluoro-5-(7-fluoro-2,3-dimethyl-1,1-dioxido-5-phenyl-2,3,4,5-tetrahydrobenzo[f][1,2,5]thiadiazepin-8-yl)benzoic acid

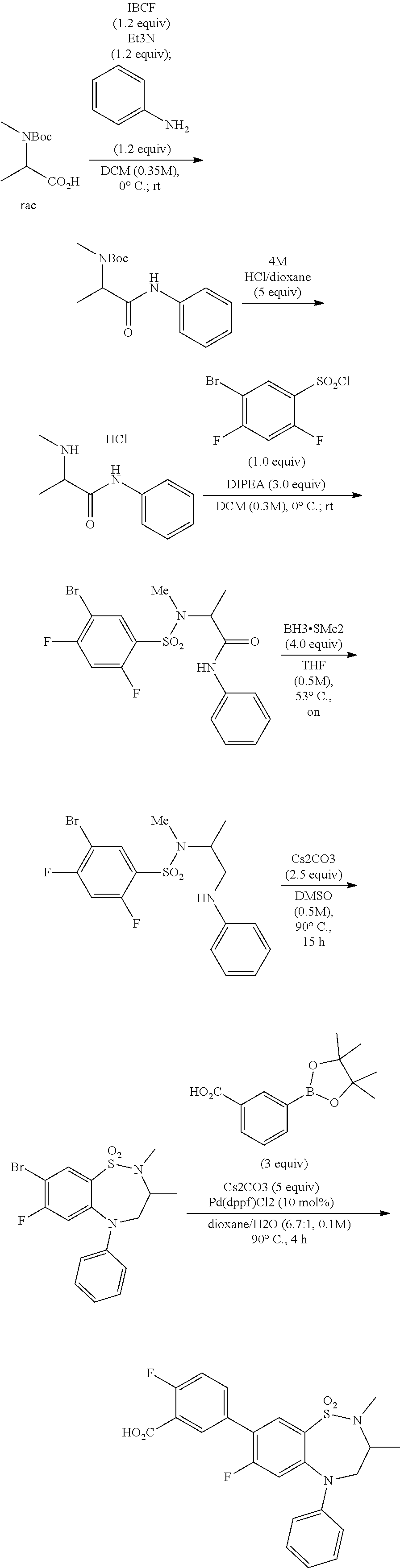

Step 1

N-(tert-butoxycarbonyl)-N-methylalanine (2.00 g, CAS #: 13734-31-1) was dissolved in dichloromethane (28.1 mL, 0.35M) in a 100 mL round-bottomed flask under a nitrogen atmosphere. The resulting solution was cooled in an ice and water bath prior to the successive addition of isobutyl chloroformate (1.55 ml, 11.8 mmol) and triethylamine (1.65 ml, 11.8 mmol). After stirring for 30 minutes, aniline (1.10 mL, 1.2 equiv) was added, and the resulting mixture was stirred for 20 h while being allowed to warm to room temperature. At this time, LCMS analysis indicated full conversion to the desired anilide and the reaction mixture was concentrated. The crude residue was purified by silica gel column chromatography to afford (tert-butyl methyl(1-oxo-1-(phenylamino)propan-2-yl)carbamate, 2.46 g, 90% yield). ESI MS m/z=301.2 [M+Na]⁺.

Step 2

Hydrochloric acid (4M in dioxanes, 5.0 equiv, 11.1 mL) was added to a 40 mL vial containing tert-butyl methyl(1-oxo-1-(phenylamino)propan-2-yl)carbamate (2.46 g). The resulting mixture was stirred for 2 h at room temperature. At this time, LCMS analysis indicated full conversion to the desired hydrochloride salt. The reaction mixture was concentrated to afford 2-(methylamino)-N-phenylpropanamide hydrochloride (1.90 g, theoretical mass) that was used directly in the next step without purification. ESI MS m/z=179.2 [M+H]⁺.

Step 3

The crude 2-(methylamino)-N-phenylpropanamide hydrochloride (1.90 g, theoretical mass) that was generated above was suspended in dichloromethane (12.6 mL, 0.7 mL). The resulting suspension was cooled in an ice and water bath prior to the addition of N,N-diisopropylethylamine (3.0 equiv, 4.63 mL). To the resulting solution was added 5-bromo-2,4-difluorobenzenesulfonyl chloride (2.58 g, 1.0 equiv, CAS #: 287172-61-6). The resulting mixture was stirred for 17 h while being allowed to warm to room temperature. At this time, LCMS analysis indicated full conversion to the desired sulfonamide. The reaction mixture was concentrated and the crude residue was purified by silica gel column chromatography to afford 2-((5-bromo-2,4-difluoro-N-methylphenyl)sulfonamido)-N-phenylpropanamide (3.00 g, 78% yield). ESI MS m/z=433.0 [M+H]⁺.

Step 4

In a 40 mL vial equipped with a stir bar under a nitrogen atmosphere, 2-((5-bromo-2,4-difluoro-N-methylphenyl)sulfonamido)-N-phenylpropanamide (3.00 g) was dissolved in tetrahydrofuran (13.9 mL, 0.5M). Borane-dimethylsulfide complex (4.0 equiv, 2.63 mL) was added, and the resulting mixture was heated at 53° C. for 12 h. At this time, LCMS analysis indicated full conversion to the desired secondary aniline, and the reaction mixture was cooled in an ice/water bath before being slowly quenched with water (3.0 mL). Upon concentration, the crude residue was purified by silica gel column chromatography to afford 5-bromo-2,4-difluoro-N-methyl-N-(1-(phenylamino)propan-2-yl)benzenesulfonamide (2.70 g, 93% yield). ESI MS m/z=419.0 [M+H]⁺.

Step 5

In a 40 mL vial equipped with a stir bar, 5-bromo-2,4-difluoro-N-methyl-N-(1-(phenylamino)propan-2-yl)benzenesulfonamide (2.70 g) was dissolved in dimethyl sulfoxide (12.9 mL, 0.5M). Cesium carbonate (2.5 equiv, 5.24 g) was added, and the resulting mixture was heated at 90° C. until full conversion to the cyclized product was determined by LCMS (15 h in this example). Note: the desired product exhibits weak ionization relative to the secondary amine starting material, and it can be difficult to judge conversion by mass signal alone. Upon full conversion, the crude reaction mixture was concentrated using a Biotage® V-10 evaporator, and the crude residue was purified by silica gel column chromatography to afford 8-bromo-7-fluoro-2,3-dimethyl-5-phenyl-2,3,4,5-tetrahydrobenzo[f][1,2,5]thiadiazepine 1,1-dioxide (1.35 g, 53% yield). ESI MS m/z=399.0 [M+H]⁺.

Step 6

In a reaction vial, 8-bromo-7-fluoro-2,3-dimethyl-5-phenyl-2,3,4,5-tetrahydrobenzo[f][1,2,5]thiadiazepine 1,1-dioxide (30 mg) was combined with 2-fluoro-5-(4,4,5,5-tetramethyl-1,3,2-dioxaborolan-2-yl)benzoic acid (60.0 mg, 3.0 equiv, CAS #: 867256-77-7), cesium carbonate (122 mg, 5.0 equiv), and [1,1′-bis(diphenylphosphino)ferrocene]dichloropalladium(II) (5.5 mg, 10 mol %, CAS #: 72287-26-4) under a nitrogen atmosphere. Dioxane (650 μL) was added, followed by water (100 μL). The vial was sealed with electrical tape and the reaction mixture was heated at 90° C. for 4 h. Upon cooling to room temperature, the reaction was quenched by the addition of formic acid (400 μL) and concentrated. The crude residue was re-dissolved in 2.0 mL of N,N-dimethylformamide, passed through a 0.2 μm syringe filter, and purified by RPHPLC to afford 2-fluoro-5-(7-fluoro-2,3-dimethyl-1,1-dioxido-5-phenyl-2,3,4,5-tetrahydrobenzo[f][1,2,5]thiadiazepin-8-yl)benzoic acid (11.4 mg, 33% yield). ESI MS m/z=459.2 [M+H]⁺. 1H NMR (400 MHz, dimethylsulfoxide-d6) δ 13.48 (br s, 1H), 8.04 (dd, J=7.1, 2.5 Hz, 1H), 7.92 (d, J=8.5 Hz, 1H), 7.90-7.86 (m, 1H), 7.47 (dd, J=10.6, 8.6 Hz, 1H), 7.29-7.25 (m, 2H), 7.20 (d, J=11.9 Hz, 1H), 6.96-6.90 (m, 3H), 4.10 (d, J=15.9 Hz, 1H), 4.00-3.92 (m, 1H), 3.57-3.50 (m, 1H), 2.65 (s, 3H), 1.26 (d, J=7.0 Hz, 3H).

The following compounds were prepared by using procedures similar to those described above:

| Example | Structure | MS | NMR |
|---|---|---|---|
| 1 | 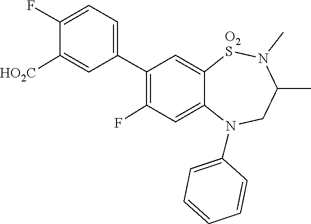 [SMILES: CC1CN(c2ccccc2)c2cc(F)c(-c3ccc(F)c(C(=O)O)c3)cc2S(=O)(=O)N1C] | [M + H]⁺ 459.2 | 1H NMR (400 MHz, dimethylsulfoxide-d6) δ 13.48 (br s, 1H), 8.04 (dd, J = 7.1, 2.5 Hz, 1H), 7.92 (d, J = 8.5 Hz, 1H), 7.90-7.86 (m, 1H), 7.47 (dd, J = 10.6, 8.6 Hz, 1H), 7.29-7.25 (m, 2H), 7.20 (d, J = 11.9 Hz, 1H), 6.96-6.90 (m, 3H), 4.10 (d, J = 15.9 Hz, 1H), 4.00-3.92 (m, 1H), 3.57-3.50 (m, 1H), 2.65 (s, 3H), 1.26 (d, J = 7.0 Hz, 3H). |
| 2 | 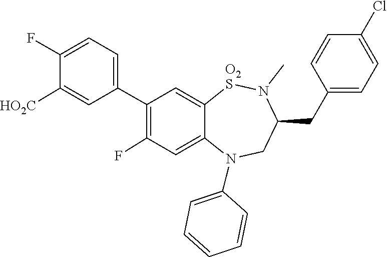 [SMILES: CN1[C@@H](Cc2ccc(Cl)cc2)CN(c2ccccc2)c2cc(F)c(-c3ccc(F)c(C(=O)O)c3)cc2S1(=O)=O] | [M + H]⁺ 569.2 | ¹H NMR (400 MHz, dimethylsulfoxide-d₆) § 13.47 (br s, 1H), 8.06-7.99 (m, 1H), 7.88 (dd, J = 16.9, 8.6 Hz, 2H), 7.45 (dd, J = 10.6, 8.6 Hz, 1H), 7.42-7.37 (m, 2H), 7.33 (d, J = 8.5 Hz, 2H), 7.25 (t, J = 7.8 Hz, 2H), 7.11 (d, J = 11.9 Hz, 1H), 6.95 (t, J = 7.3 Hz, 1H), 6.86-6.84 (m, 2H), 4.15 (d, J = 15.7 Hz, 1H), 4.05-3.99 (m, 1H), 3.78 (s, 1H), 3.01-2.89 (m, 2H), 2.60 (s, 3H) |
| 3 | 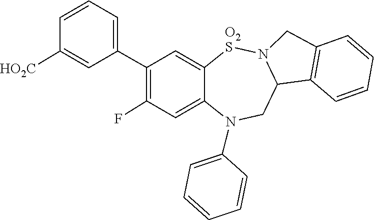 [SMILES: O=C(O)c1cccc(-c2cc3c(cc2F)N(c2ccccc2)CC2c4ccccc4CN2S3(=O)=O)c1] | [M + H]⁺ 501.2 | |
| 4 | 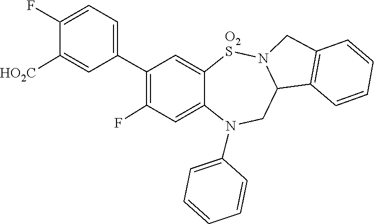 [SMILES: O=C(O)c1cc(-c2cc3c(cc2F)N(c2ccccc2)CC2c4ccccc4CN2S3(=O)=O)ccc1F] | [M + H]⁺ 519.2 | |
| 5 | 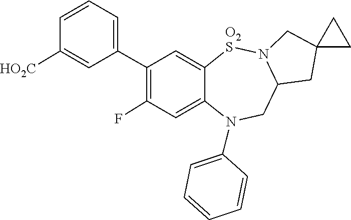 [SMILES: O=C(O)c1cccc(-c2cc3c(cc2F)N(c2ccccc2)CC2CC4(CC4)CN2S3(=O)=O)c1] | [M + H]⁺ 479.1 | |
| 6 | 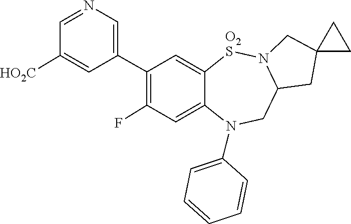 [SMILES: O=C(O)c1cncc(-c2cc3c(cc2F)N(c2ccccc2)CC2CC4(CC4)CN2S3(=O)=O)c1] | [M + H]⁺ 480.2 | |
| 7 | 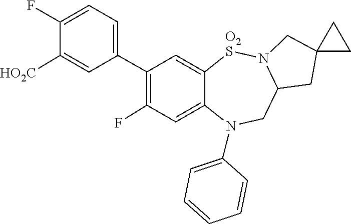 [SMILES: O=C(O)c1cc(-c2cc3c(cc2F)N(c2ccccc2)CC2CC4(CC4)CN2S3(=O)=O)ccc1F] | [M + H]⁺ 497.2 | |
| 8 | 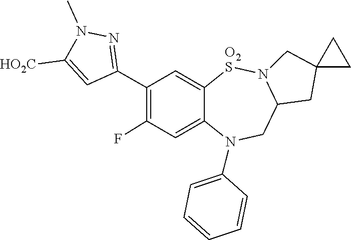 [SMILES: Cn1nc(-c2cc3c(cc2F)N(c2ccccc2)CC2CC4(CC4)CN2S3(=O)=O)cc1C(=O)O] | [M + H]⁺ 483.2 | ¹H NMR (400 MHz, dimethylsulfoxide-d₆) δ 13.63, 8.44 (d, J = 8.3 Hz, 1H), 7.28-7.24 (m, 3H), 7.13 (d, J = 3.9 Hz, 1H), 6.94-6.85 (m, 3H), 5.76 (s, 1H), 4.43 (dd, J = 15.9, 2.2 Hz, 1H), 4.19 (s, 3H), 4.15-3.99 (m, 1H), 3.06 (d, J = 9.5 Hz, 1H), 2.35-2.18 (m, 1H), 1.73-1.54 (m, 1H), 0.75-0.30 (m, 4H) |
| 9 | 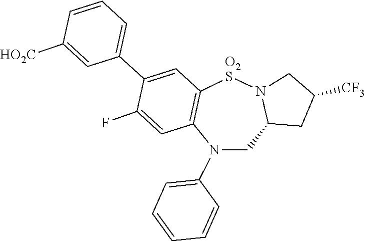 [SMILES: O=C(O)c1cccc(-c2cc3c(cc2F)N(c2ccccc2)C[C@H]2C[C@@H](C(F)(F)F)CN2S3(=O)=O)c1] | [M + H]⁺ 521.2 | |
| 10 | 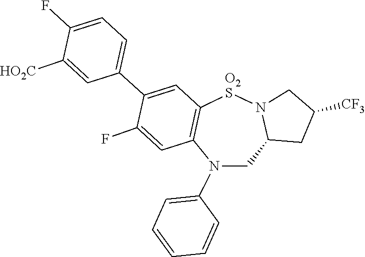 [SMILES: O=C(O)c1cc(-c2cc3c(cc2F)N(c2ccccc2)C[C@H]2C[C@@H](C(F)(F)F)CN2S3(=O)=O)ccc1F] | [M + H]⁺ 539.2 | |
| 11 | 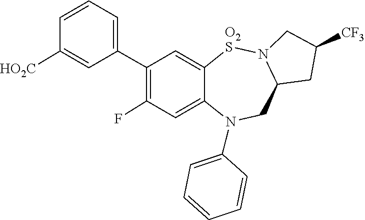 [SMILES: O=C(O)c1cccc(-c2cc3c(cc2F)N(c2ccccc2)C[C@@H]2C[C@H](C(F)(F)F)CN2S3(=O)=O)c1] | [M + H]⁺ 521.2 | |

### Ex.12: Synthesis of 3-(7-cyano-2,3-dimethyl-1,1-dioxido-5-phenyl-2,3,4,5-tetrahydrobenzo[f][1,2,5]thiadiazepin-8-yl)benzoic acid

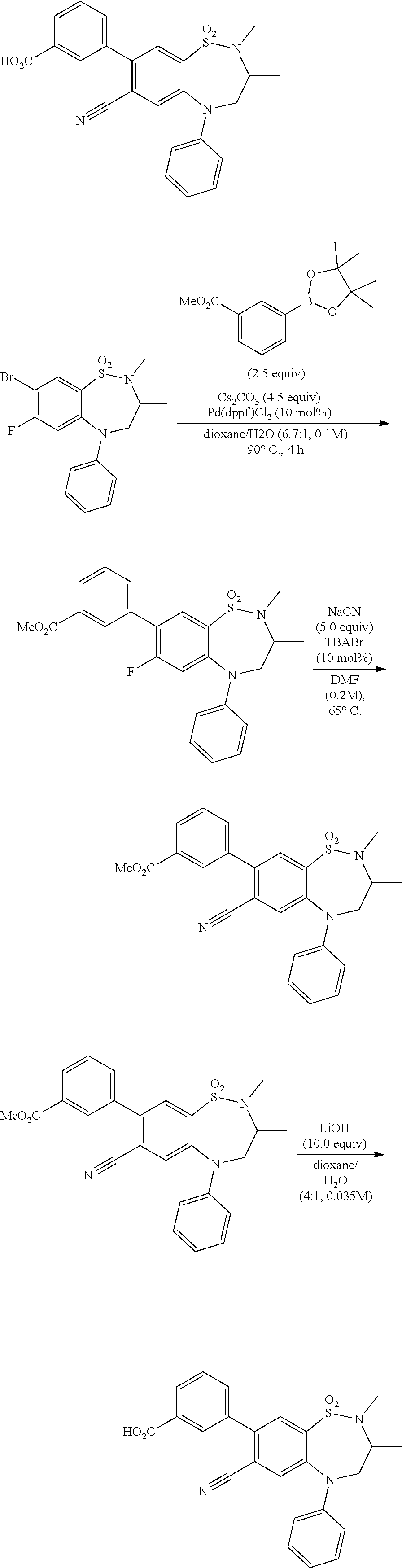

Step 1

In a 40 mL vial equipped with a stir bar, 8-bromo-7-fluoro-2,3-dimethyl-5-phenyl-2,3,4,5-tetrahydrobenzo[f][1,2,5]thiadiazepine 1,1-dioxide (200.0 mg) was combined with methyl 3-(4,4,5,5-tetramethyl-1,3,2-dioxaborolan-2-yl)benzoate (328.0 mg, 2.5 equiv, CAS #: 480425-35-2), cesium carbonate (734.0 mg, 4.5 equiv), and [1,1′-bis(diphenylphosphino)ferrocene]dichloropalladium(II) (36.7 mg, 10 mol %, CAS #: 72287-26-4) under a nitrogen atmosphere. Dioxane (4.4 mL) and water (650 μL) were added and the vial was sealed with electrical tape. The mixture was heated at 90° C. for 4 h. Upon concentration, the crude residue was purified by silica gel column chromatography to afford methyl 3-(7-fluoro-2,3-dimethyl-1,1-dioxido-5-phenyl-2,3,4,5-tetrahydrobenzo[f][1,2,5]thiadiazepin-8-yl)benzoate in quantitative yield. ESI MS m/z=455.2 [M+H]⁺.

Step 2

Sodium cyanide (13.5 mg) and tetrabutylammonium bromide were combined neat in a 4 mL vial equipped with a stir bar Next, methyl 3-(7-fluoro-2,3-dimethyl-1,1-dioxido-5-phenyl-2,3,4,5-tetrahydrobenzo[f][1,2,5]thiadiazepin-8-yl)benzoate (25.0 mg) was added to the vial as a solution in NN-dimethylformamide (275 μL, 0.2M). The vial was sealed and heated at 65° C. for 4 h. Upon cooling to room temperature, the mixture was diluted with water and extracted several times with methylene chloride. The organic layers were passed through a phase separator and concentrated. The residue was purified by RPHPLC to afford methyl 3-(7-cyano-2,3-dimethyl-1,1-dioxido-5-phenyl-2,3,4,5-tetrahydrobenzo[f][1,2,5]thiadiazepin-8-yl)benzoate (10.7 mg, 42% yield) ESI MS mn/z=462.2 [M+H]⁺.

Step 3

Methyl 3-(7-cyano-2.3-dimethyl-1,1-dioxido-5-phenyl-2,3,4,5-tetrahydrobenzo[f][1,2,5]thiadiazepin-8-yl)benzoate (10.7 mg) was dissolved in dioxane (530 μL) and water was added (130 μL) followed by lithium hydroxide (5.6 mg, 10.0 equiv). The mixture was stirred for 16 h at rt, quenched with formic acid (400 μL), and concentrated. The crude residue was re-dissolved in N,N-dimethylformamide (2.0 mL), passed through a 0.2 pun syringe filter, and purified by RPHPLC to afford 3-(7-cyano-2,3-dimethyl-1,1-dioxido-5-phenyl-2,3,4,5-tetrahydrobenzo[f][1,2,5]thiadiazepin-8-yl)benzoic acid (4.1 mg, 40% yield). ESI MS m/z=448.2 [M+H].

The following compounds were prepared by using a procedure similar to that described above:

| Ex- | | |
|---|---|---|
| am- | | |
| ple | Structure | MS |
| 12 | 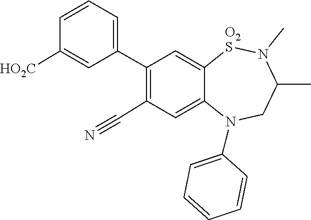 [SMILES: CC1CN(c2ccccc2)c2cc(C#N)c(-c3cccc(C(=O)O)c3)cc2S(=O)(=O)N1C] | 448.2 |
| 13 | 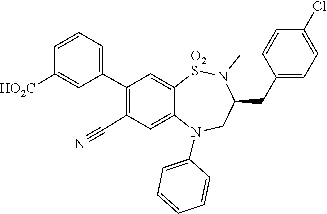 [SMILES: CN1[C@@H](Cc2ccc(Cl)cc2)CN(c2ccccc2)c2cc(C#N)c(-c3cccc(C(=O)O)c3)cc2S1(=O)=O] | [M + H] 558.2 |
| 14 | 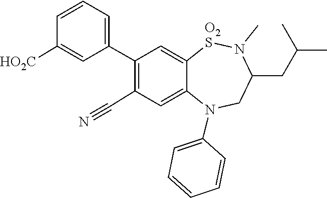 [SMILES: CC(C)CC1CN(c2ccccc2)c2cc(C#N)c(-c3cccc(C(=O)O)c3)cc2S(=O)(=O)N1C] | [M + H] 490.2 |

### Ex.15: Synthesis of 3-(7-methoxy-2,3-dimethyl-1,1-dioxido-5-phenyl-2,3,4,5-tetrahydrobenzo[f][1,2,5]thiadiazepin-8-yl)benzoic acid

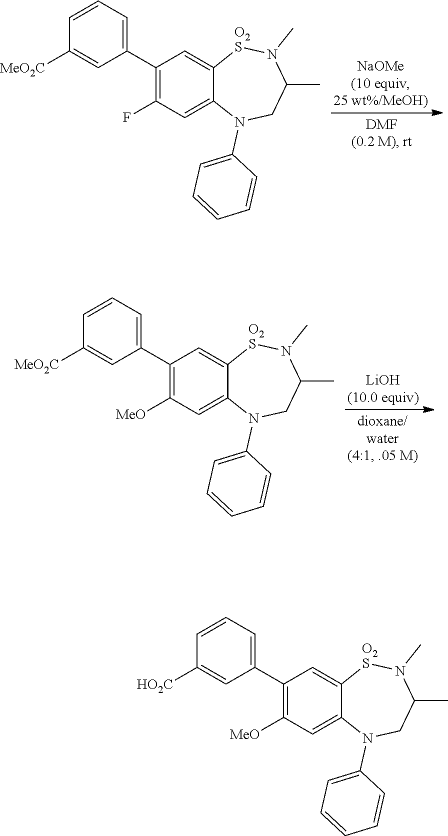 [SMILES: COC(=O)c1cccc(-c2cc3c(cc2F)N(c2ccccc2)CC(C)N(C)S3(=O)=O)c1 ; COC(=O)c1cccc(-c2cc3c(cc2OC)N(c2ccccc2)CC(C)N(C)S3(=O)=O)c1 ; COc1cc2c(cc1-c1cccc(C(=O)O)c1)S(=O)(=O)N(C)C(C)CN2c1ccccc1]

Step 1

Methyl 3-(7-fluoro-2,3-dimethyl-1,1-dioxido-5-phenyl-2,3,4,5-tetrahydrobenzo[f][1,2,5]thiadiazepin-8-yl)benzoate (30.0 mg) was dissolved in N,N-dimethylformamide (330 μL, 0.2M) in a 4 mL vial equipped with a stir bar. A solution of sodium methoxide (25 wt % in methanol, 151 μL, 10.0 equiv) was added, and the resulting mixture was stirred at room temperature for 3 h. Upon full conversion, the mixture was quenched with formic acid (400 μL) and concentrated. The crude residue was re-dissolved in N,N-dimethylformamide (2.0 mL), passed through a 0.2 μm syringe filter, and purified by RPHPLC to afford methyl 3-(7-methoxy-2,3-dimethyl-1,1-dioxido-5-phenyl-2,3,4,5-tetrahydrobenzo[f][1,2,5]thiadiazepin-8-yl)benzoate (20.6 mg, 67% yield). ESI MS m/z=467.2 [M+H]⁺.

Step 2

In a reaction vial, methyl 3-(7-methoxy-2,3-dimethyl-1,1-dioxido-5-phenyl-2,3,4,5-tetrahydrobenzo[f][1,2,5]thiadiazepin-8-yl)benzoate (20.6 mg) was dissolved in dioxane (710 μL) and water (180 μL). Lithium hydroxide (10.6 mg, 10.0 equiv) was added, and the mixture was stirred at room temperature for 16 h. Upon full conversion, as determined by LCMS analysis, the reaction mixture was quenched with formic acid (400 μL) and concentrated. The crude residue was re-dissolved in N,N-dimethylformamide (2.0 mL), passed through a 0.2 μm syringe filter and purified by RPHPLC to afford 3-(7-methoxy-2,3-dimethyl-1,1-dioxido-5-phenyl-2,3,4,5-tetrahydrobenzo[f][1,2,5]thiadiazepin-8-yl)benzoic acid (5.6 mg, 28%). ESI MS m/z=453.2 [M+H]⁺.

Examples 16 and 17 were prepared by using a procedure similar to that described above.

| Ex- | | |
|---|---|---|
| am- | | |
| ple | Structure | MS |
| 15 | 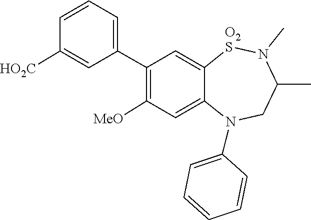 [SMILES: COc1cc2c(cc1-c1cccc(C(=O)O)c1)S(=O)(=O)N(C)C(C)CN2c1ccccc1] | [M + H]⁺ 453.2 |
| 16 | 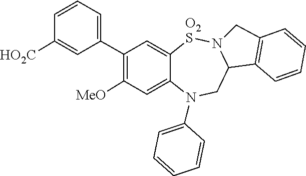 [SMILES: COc1cc2c(cc1-c1cccc(C(=O)O)c1)S(=O)(=O)N1Cc3ccccc3C1CN2c1ccccc1] | [M + H]⁺ 513.2 |
| 17 | 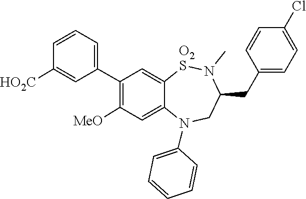 [SMILES: COc1cc2c(cc1-c1cccc(C(=O)O)c1)S(=O)(=O)N(C)[C@@H](Cc1ccc(Cl)cc1)CN2c1ccccc1] | [M + H]⁺ 563.2 |

### Ex.18: Synthesis of 3-(8-(methylthio)-5,5-dioxido-10-phenyl-1,10,11,11a-tetrahydro-3H-spiro[benzo[f]pyrrolo[1,2-b][1,2,5]thiadiazepine-2,1′-cyclopropan]-7-yl)benzoic acid

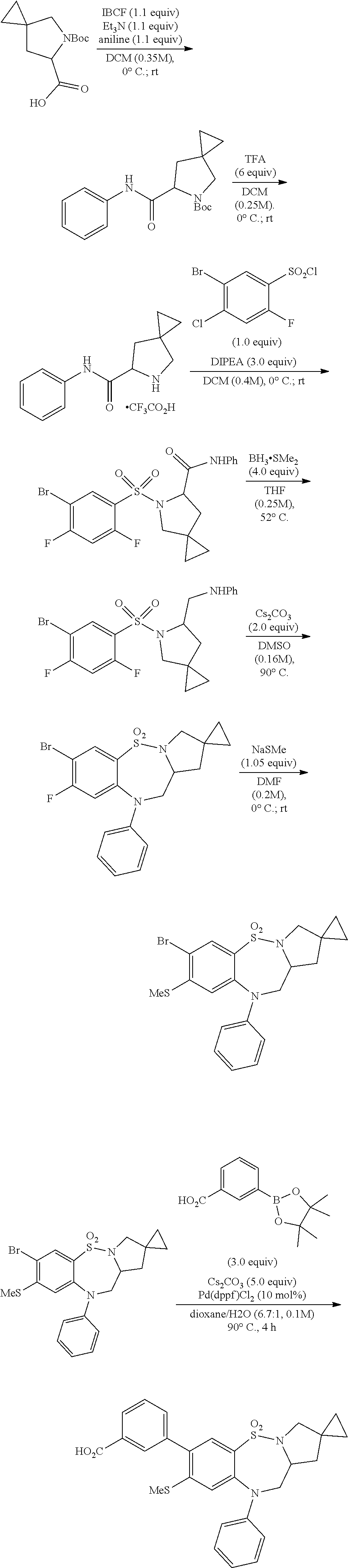

Step 1

In a 250 mL round-bottomed flask, 5-(tert-butoxycarbonyl)-5-azaspiro[2.4]heptane-6-carboxylic acid (5.00 g, CAS #: 1454843-77-6) was dissolved in dichloromethane (59.2 mL, 0.35M). The solution was cooled in an ice and water bath, and isobutyl chloroformate (3.0 mL, 1.1 equiv) was added followed by triethylamine (3.2 mL, 1.1 equiv). The resulting solution was stirred for 30 minutes before aniline (2.1 mL, 1.1 equiv) was added. The mixture was stirred for 24 h while being allowed to warm to room temperature. Upon completion, the reaction mixture was diluted with saturated aqueous sodium bicarbonate and the aqueous phase was extracted several times with dichloromethane. The combined organic layers were dried over magnesium sulfate then concentrated. Upon concentration, the crude residue was purified by silica gel column chromatography to afford tert-butyl 6-(phenylcarbamoyl)-5-azaspiro[2.4]heptane-5-carboxylate (5.22 g, 80% yield). ESI MS m/z=339.2 [M+H]⁺.

Step 2

To a 250 mL round-bottomed flask equipped with a stir bar was added tert-butyl 6-(phenylcarbamoyl)-5-azaspiro[2.4]heptane-5-carboxylate (5.22 g) and dichloromethane (66.0 mL, 0.25M). The resulting solution was cooled in an ice and water bath, and trifluoroacetic acid was added (7.6 mL, 6.0 equiv). The reaction mixture was stirred for 24 h, and additional trifluoroacetic acid (3.8 mL, 3.0 equiv) was added. After stirring for 16 h, the reaction mixture was concentrated to afford crude N-phenyl-5-azaspiro[2.4]heptane-6-carboxamide 2,2,2-trifluoroacetate, which was used without further purification. ESI MS m/z=217.2 [M+H]⁺.

Step 3

In a 250 mL round-bottomed flask equipped with a stir N-phenyl-5-azaspiro[2.4]heptane-6-carboxamide 2,2,2-trifluoroacetate (8.27 g) was dissolved in dichloromethane (63 mL, 0.4M). The resulting solution was cooled in an ice and water bath and N,N-diisopropylethylamine (13.1 mL, 3.0 equiv) was added followed by 5-bromo-2,4-difluorobenzenesulfonyl chloride (7.3 g, 1.0 equiv, CAS #: 287172-61-6). After being allowed to stir for 18 h while warming to room temperature, the reaction mixture was concentrated and the crude residue was purified by silica gel column chromatography to afford 5-((5-bromo-2,4-difluorophenyl)sulfonyl)-N-phenyl-5-azaspiro[2.4]heptane-6-carboxamide (5.59 g, 47%). ESI MS m/z=471.2 [M+H]⁺.

Step 4

In a 250 mL round-bottomed flask equipped with a stir bar and reflux condenser, 5-((5-bromo-2,4-difluorophenyl)sulfonyl)-N-phenyl-5-azaspiro[2.4]heptane-6-carboxamide (5.59 g) was dissolved in tetrahydrofuran (59 mL, 0.2M). Borane-dimethyl sulfide complex (4.50 mL, 4.0 equiv) was added, and the resulting mixture was heated at 60° C. for 19 h. Upon cooling to room temperature, the reaction mixture was quenched slowly with 10 mL of water and concentrated. The crude residue was purified by silica gel column chromatography to afford N-((5-((5-bromo-2,4-difluorophenyl)sulfonyl)-5-azaspiro[2.4]heptan-6-yl)methyl)aniline (4.72 g, 87%). ESI MS m/z=457.0 [M+H]⁺.

Step 5

In a 250 mL round-bottomed flask equipped with a stir bar and reflux condensor N-((5-((5-bromo-2,4-difluorophenyl)sulfonyl)-5-azaspiro[2.4]heptan-6-yl)methyl)aniline (4.72 g) was dissolved in dimethyl sulfoxide (65 mL, 0.16M). Cesium carbonate (6.73 g, 2.0 equiv) was added, and the resulting mixture was heated at heated at 90° C. for 6.5 h. Upon cooling to room temperature, the reaction mixture was diluted with methyl tert-butyl ether and water and the layers were separated. The aqueous phase was further extracted methyl tert-butyl ether and the combined organic phase was dried over magnesium sulfate. Upon concentration, the crude residue was purified by silica gel column chromatography to afford 7-bromo-8-fluoro-10-phenyl-1,10,11,11a-tetrahydro-3H-spiro[benzo[f]pyrrolo[1,2-b][1,2,5]thiadiazepine-2,1′-cyclopropane]5,5-dioxide (3.48 g, 77%). ESI MS m/z=437.0 [M+H]⁺.

Step 6

A 4 mL vial equipped with a stir bar was charged with sodium thiomethoxide (16.8 mg, 1.05 equiv) and N,N-dimethylformamide (1.1 mL, 0.2M). The resulting suspension was cooled in an ice and water bath prior to the addition of 7-bromo-8-fluoro-10-phenyl-1,10,11,11a-tetrahydro-3H-spiro[benzo[f]pyrrolo[1,2-b][1,2,5]thiadiazepine-2,1′-cyclopropane]5,5-dioxide (100 mg). After being stirred for 14 h while being warmed to room temperature, additional sodium thiomethoxide (10.0 mg) was added. After 1 h, the reaction mixture was quenched by the addition of formic acid (500 μL) and concentrated under a stream of nitrogen. The crude residue was purified via silica gel column chromatography to afford 7-bromo-8-(methylthio)-10-phenyl-1,10,11,11a-tetrahydro-3H-spiro[benzo[f]pyrrolo[1,2-b][1,2,5]thiadiazepine-2,1′-cyclopropane]5,5-dioxide (82.5 mg, 78%). ESI MS m/z=465.0 [M+H]⁺.

Step 7

In a 4 mL vial equipped with a stir bar, 7-bromo-8-(methylthio)-10-phenyl-1,10,11,11a-tetrahydro-3H-spiro[benzo[f]pyrrolo[1,2-b][1,2,5]thiadiazepine-2,1′-cyclopropane]5,5-dioxide (35.0 mg), 3-(4,4,5,5-tetramethyl-1,3,2-dioxaborolan-2-yl)benzoic acid (56.0 mg, 3.0 equiv, CAS #: 269409-73-6), cesium carbonate (123.0 mg, 5.0 equiv), and [1,1′-bis(diphenylphosphino)ferrocene]dichloropalladium(II) (5.5 mg, 10 mol %, CAS #:

72287-26-4) were combined neat under a nitrogen atmosphere. Dioxane (650 μL) and water (100 μL) were added, and the vial was sealed with electrical tape. The reaction mixture was heated at 90° C. for 5 h. Upon cooling to room temperature, the reaction mixture was quenched with formic acid (400 μL) and concentrated. The crude residue was re-dissolved in N,N-dimethylformamide (2.0 mL), passed through a 0.2 m syringe filter, and purified by RPHIPLC to afford 3-(8-(methylthio)-5,5-dioxido-10-phenyl-1,10,11,11a-tetrahydro-3H-spiro[benzo[f]pyrrolo[1,2-b][1,2,5]thiadiazepine-2,1′-cyclopropan]-7-yl)benzoic acid (22.2 mg, 580 yield). ESI MS m/z=507.2 [M+H]⁺. ¹H NMR (400 MHz, dimethylsulfoxide-d6) δ 13.14 (br s, 1H), 8.05-7.94 (m, 2H), 7.72 (dt, J=7.7, 1.6 Hz, 1H), 7.62 (t, J=7.6 Hz, 1H), 7.57 (s, 1H), 7.27-7.15 (m, 3H), 6.86-6.75 (m, 3H), 4.41 (dd, J=15.8, 2.2 Hz, 1H), 4.18-4.14 (m, 1H), 3.04 (d, J=9.5 Hz, 1H), 2.39-2.32 (i, 1H), 2.35 (s, 3H), 1.62 (d, J=12.4 Hz, 1H), 0.72-0.44 (in, 4H).

Examples 19-24 were prepared using procedures similar to that described above.

| Example | Structure | MS | NMR |
|---|---|---|---|
| 18 | 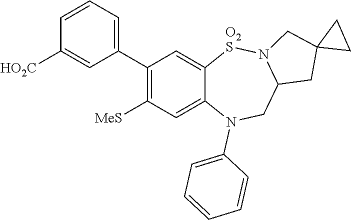 [SMILES: CSc1cc2c(cc1-c1cccc(C(=O)O)c1)S(=O)(=O)N1CC3(CC3)CC1CN2c1ccccc1] | [M + H]⁺ 507.2 | |
| 19 | 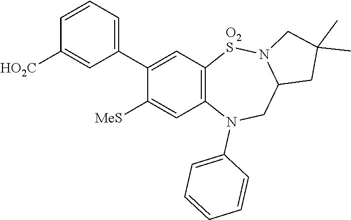 [SMILES: CSc1cc2c(cc1-c1cccc(C(=O)O)c1)S(=O)(=O)N1CC(C)(C)CC1CN2c1ccccc1] | [M + H]⁺ 509.2 | 1H NMR (400 MHz, dimethyl- sulfoxide-d₆) δ 13.15 (br s, 1H), 8.0-7.95 (m, 2H), 7.70-7.59 (m, 2H), 7.54 (s, 1H), 7.30-7.26 (m, 1H), 7.02-6.88 (m, 4H), 4.36 (d, J = 15.7 Hz, 1H), 4.11-4.04 (m, 1H), 3.15 (d, J = 8.8 Hz, 1H), 2.96 (d, J = 8.9 Hz, 1H), 2.66 (s, 3H), 1.97 (dd, J = 12.9, 8.0 Hz, 1H), 1.49 (dd, J = 13.0, 6.3 Hz, 1H), 1.11 (s, 3H), 1.03 (s, 3H) |
| 20 | 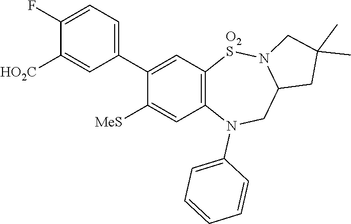 [SMILES: CSc1cc2c(cc1-c1ccc(F)c(C(=O)O)c1)S(=O)(=O)N1CC(C)(C)CC1CN2c1ccccc1] | [M + H]⁺ 527.2 | |
| 21 | 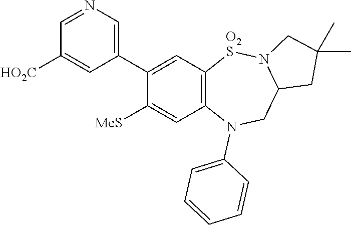 [SMILES: CSc1cc2c(cc1-c1cncc(C(=O)O)c1)S(=O)(=O)N1CC(C)(C)CC1CN2c1ccccc1] | [M + H]⁺ 510.2 | |
| 22 | 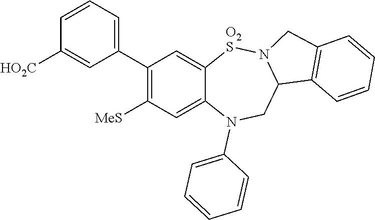 [SMILES: CSc1cc2c(cc1-c1cccc(C(=O)O)c1)S(=O)(=O)N1Cc3ccccc3C1CN2c1ccccc1] | [M + H]⁺ 529.2 | |
| 23 | 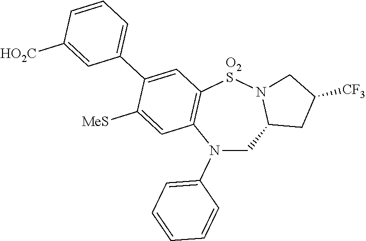 [SMILES: CSc1cc2c(cc1-c1cccc(C(=O)O)c1)S(=O)(=O)N1C[C@H](C(F)(F)F)C[C@@H]1CN2c1ccccc1] | [M + H]⁺ 549.2 | |
| 24 | 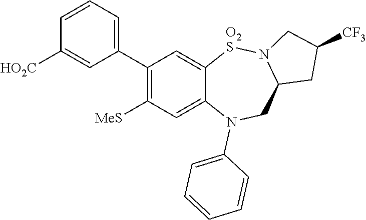 [SMILES: CSc1cc2c(cc1-c1cccc(C(=O)O)c1)S(=O)(=O)N1C[C@@H](C(F)(F)F)C[C@H]1CN2c1ccccc1] | [M + H]⁺ 549.2 | |

### Ex.25: Synthesis of(R)-5-(7-chloro-3-cyclohexyl-2-methyl-1,1-dioxido-5-phenyl-2,3,4,5-tetrahydrobenzo[f][1,2,5]thiadiazepin-8-yl)-2-fluorobenzoic acid

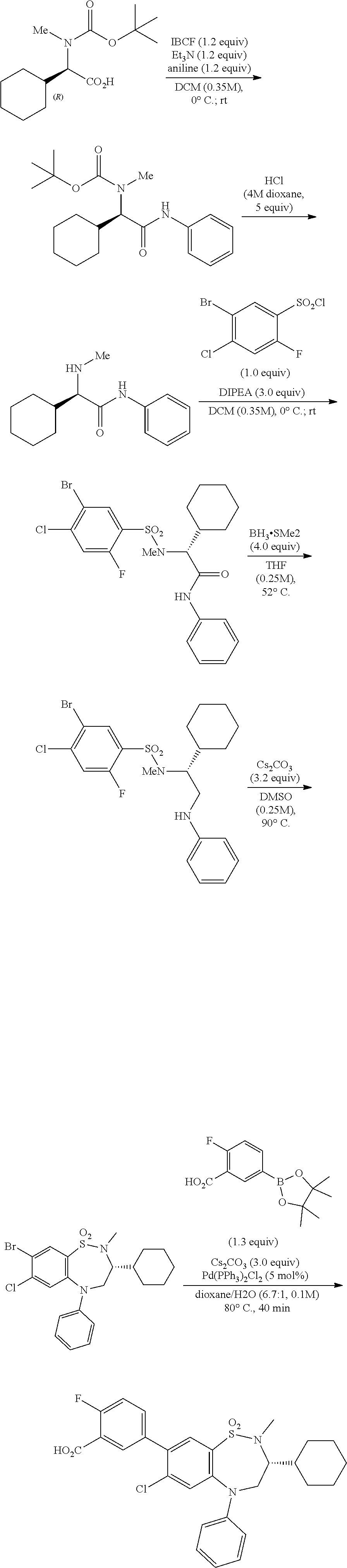

Step 1

In a 40 mL vial equipped with a stir bar, (R)-2-((tert-butoxycarbonyl)(methyl)amino)-2-cyclohexylacetic acid (1.00 g, CAS: 287210-86-0) was dissolved in dichloromethane (11 mL, 0.35M). The resulting solution was cooled in an ice and water bath. Next, isobutyl chloroformate (580 μL, 1.2 equiv) was added, followed by triethylamine (620 μL, 1.2 equiv). The resulting mixture was stirred at the same temperature for 30 minutes prior to the addition of aniline (400 μL). After stirring for 16 h while being allowed to warm to room temperature, the reaction mixture was concentrated and the crude residue was purified by silica gel column chromatography to afford tert-butyl (R)-(1-cyclohexyl-2-oxo-2-(phenylamino)ethyl)(methyl)carbamate (991.9 mg, 78%). ESI MS m/z=369.2 [M+Na]*.

Step 2

Hydrochloric acid (4M dioxane, 3.6 mL, 5.0 equiv) was added to a 40 mL vial containing tert-butyl (R)-(1-cyclohexyl-2-oxo-2-(phenylamino)ethyl)(methyl)carbamate (991.9 mg) and a stir bar. The reaction mixture was stirred for 2 h and concentrated to afford (R)-2-cyclohexyl-2-(methylamino)-N-phenylacetamide hydrochloride (810 mg, theoretical mass) which was used directly without purification. ESI MS m/z=247.2 [M+H]⁺.

Step 3

In a 40 mL vial equipped with a stir bar, (R)-2-cyclohexyl-2-(methylamino)-N-phenylacetamide hydrochloride (810 mg) produced above was suspended in dichloromethane (8.2 mL, 0.35M). The suspension was cooled in an ice and water bath and N,N-diisopropylethylamine (1.5 mL, 3.0 equiv) was added, followed by 5-bromo-4-chloro-2-fluorobenzenesulfonyl chloride (882 mg, 1.0 equiv, CAS #: 1070972-67-6). The resulting solution was stirred for 3 h and then concentrated. The crude residue was purified by silica gel column chromatography (cyclohexane/ethyl acetate) to afford (R)-2-((5-bromo-4-chloro-2-fluoro-N-methylphenyl)sulfonamido)-2-cyclohexyl-N-phenylacetamide (1.14 g, 77%). ESI MS m/z=517.0 [M+H]⁺.

Step 4

In a 40 mL vial equipped with a stir bar, (R)-2-((5-bromo-4-chloro-2-fluoro-N-methylphenyl)sulfonamido)-2-cyclohexyl-N-phenylacetamide (1.14 g) was dissolved in tetrahydrofuran under a nitrogen atmosphere. Borane-dimethylsulfide complex was then added (4.0 equiv, 835 μL), and the reaction mixture was heated at 52° C. for 24 h. Upon cooling to room temperature, the reaction was slowly quenched with water (1.0 mL) and concentrated. The crude residue was purified by silica gel column chromatography (cyclohexane/ethyl acetate) to afford (R)-5-bromo-4-chloro-N-(1-cyclohexyl-2-(phenylamino)ethyl)-2-fluoro-N-methylbenzenesulfonamide (789 mg, 71% yield). ESI MS m/z=503.0 [M+H]⁺.

Step 5

In a 40 mL vial equipped with a stir bar, (R)-5-bromo-4-chloro-N-(1-cyclohexyl-2-(phenylamino)ethyl)-2-fluoro-N-methylbenzenesulfonamide (789 mg) was combined neat with cesium carbonate (1.63 g, 3.2 equiv). Next, dimethyl sulfoxide (6.3 mL, 0.25M) was added, and the resulting mixture was heated at 90° C. in the sealed vial for 4 h. Upon cooling to room temperature, the reaction mixture was concentrated on a Biotage® V-10 evaporator and purified by silica gel column chromatography (cyclohexane/ethyl acetate) to afford (R)-8-bromo-7-chloro-3-cyclohexyl-2-methyl-5-phenyl-2,3,4,5-tetrahydrobenzo[f][1,2,5]thiadiazepine 1,1-dioxide (633 mg, 84% yield). [M+H], 482.8.

Step 6

In a 4 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (40 mg), 2-fluoro-5-(4,4,5,5-tetramethyl-1,3,2-dioxaborolan-2-yl)benzoic acid (28.6 mg, 1.3 equiv, CAS #: 867256-77-7), cesium carbonate (81.0 mg, 3.0 equiv), and bis(triphenylphosphine)palladium(II) chloride (2.9 mg, 5 mol %, CAS #: 13965-03-2) were combined neat under a nitrogen atmosphere. Dioxane (720 μL) and water (110 μL) were added and the vial was sealed with electrical tape. The mixture was heated at 80° C. for 40 min. Upon cooling to room temperature, the reaction mixture as quenched with formic acid (400 μL) and concentrated. The crude residue was re-dissolved in N,N-dimethylformamide, passed through a 0.2 m syringe filter and purified by RPTIPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-fluorobenzoic acid (12.2 mg, 2700 yield. ESI MS m/z=543.2 [M+H]⁺.

Examples 26-58 were prepared using procedures similar to that described above.

| Example | Structure | MS | NMR |
|---|---|---|---|
| 25 | 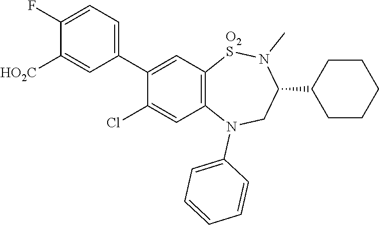 [SMILES: CN1[C@H](C2CCCCC2)CN(c2ccccc2)c2cc(Cl)c(-c3ccc(F)c(C(=O)O)c3)cc2S1(=O)=O] | [M + H]⁺ 543.2 | |
| 26 | 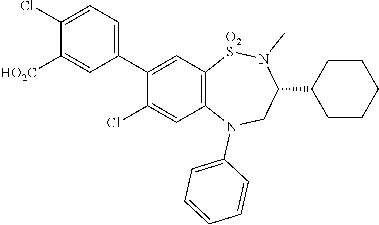 [SMILES: CN1[C@H](C2CCCCC2)CN(c2ccccc2)c2cc(Cl)c(-c3ccc(Cl)c(C(=O)O)c3)cc2S1(=O)=O] | [M + H]⁺ 559.0 | |
| 27 | 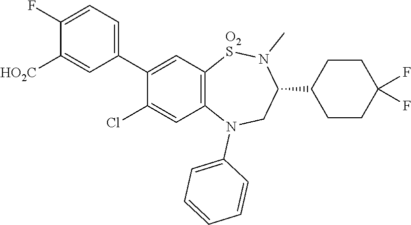 [SMILES: CN1[C@H](C2CCC(F)(F)CC2)CN(c2ccccc2)c2cc(Cl)c(-c3ccc(F)c(C(=O)O)c3)cc2S1(=O)=O] | [M + H]⁺ 579.13 | |
| 28 | 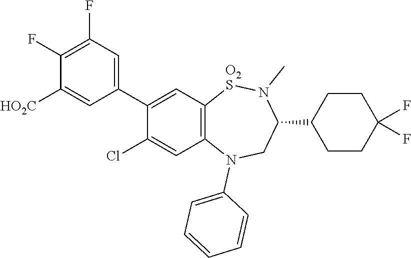 [SMILES: CN1[C@H](C2CCC(F)(F)CC2)CN(c2ccccc2)c2cc(Cl)c(-c3cc(F)c(F)c(C(=O)O)c3)cc2S1(=O)=O] | [M + H]⁺ 595.1 | |
| 29 | 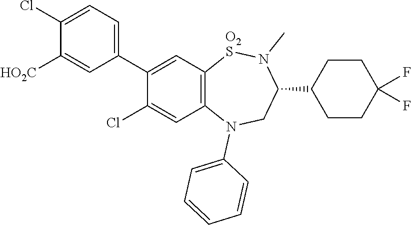 [SMILES: CN1[C@H](C2CCC(F)(F)CC2)CN(c2ccccc2)c2cc(Cl)c(-c3ccc(Cl)c(C(=O)O)c3)cc2S1(=O)=O] | [M + H]⁺ 595.1 | |
| 30 | 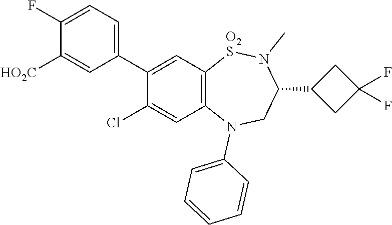 [SMILES: CN1[C@H](C2CC(F)(F)C2)CN(c2ccccc2)c2cc(Cl)c(-c3ccc(F)c(C(=O)O)c3)cc2S1(=O)=O] | [M + H]⁺ 551.1 | |
| 31 | 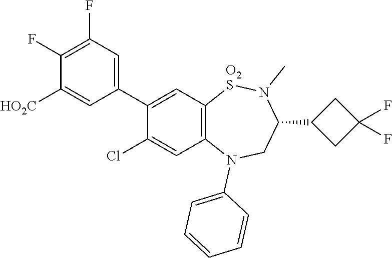 [SMILES: CN1[C@H](C2CC(F)(F)C2)CN(c2ccccc2)c2cc(Cl)c(-c3cc(F)c(F)c(C(=O)O)c3)cc2S1(=O)=O] | [M + H]⁺ 569.1 | |
| 32 | 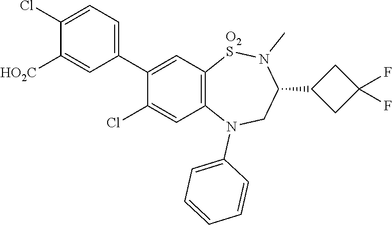 [SMILES: CN1[C@H](C2CC(F)(F)C2)CN(c2ccccc2)c2cc(Cl)c(-c3ccc(Cl)c(C(=O)O)c3)cc2S1(=O)=O] | [M + H]⁺ 567.07 | |
| 33 | 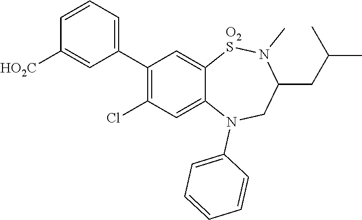 [SMILES: CC(C)CC1CN(c2ccccc2)c2cc(Cl)c(-c3cccc(C(=O)O)c3)cc2S(=O)(=O)N1C] | [M + H]⁺ 499.20 | ¹H NMR (400 MHz, dimethyl- sulfoxide-d₆) δ 13.17 (br s, 1H), 8.04-8.02 (m, 2H), 7.80-7.78 (m, 2H), 7.65 (t, J = 7.7 Hz, 1H), 7.46 (s, 1H), 7.27 (t, J = 7.8 Hz, 2H), 6.92-6.88 (m, 1H), 4.10 (d, J = 16.1 Hz, 1H), 3.89 (s, 1H), 3.46 (s, 1H), 2.61 (s, 3H), 1.76-1.65 (m, 1H), 1.60- 1.53 (m, 1H), 1.40-1.33 (m, 1H), 0.93 (d, J = 6.6 Hz, 3H), 0.89 (d, J = 6.5 Hz, 3H) |
| 34 | 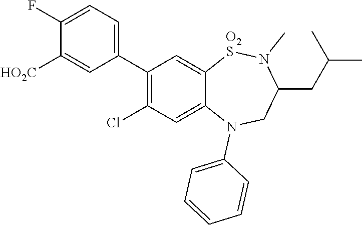 [SMILES: CC(C)CC1CN(c2ccccc2)c2cc(Cl)c(-c3ccc(F)c(C(=O)O)c3)cc2S(=O)(=O)N1C] | [M + H]⁺ 517.2 | ¹H NMR (400 MHz,dimethyl- sulfoxide-d₆) δ 13.45 (s, 1H), 7.95 (dd, J = 7.0, 2.6 Hz, 1H), 7.85-7.72 (m, 2H), 7.52-7.39 (m, 2H), 7.32-7.22 (m, 2H), 6.89 (d, J = 7.2 Hz, 3H), 4.10 (d, J = 16.2 Hz, 1H), 3.89 (s, 1H), 3.46 (s, 1H), 2.60 (s, 3H), 1.75-1.65 (m, 1H), 1.60-1.52 (m, 1H), 1.39-1.32 (m, 1H), 0.92 (d, J = 6.6 Hz, 3H), 0.89 (d, J = 6.5 Hz, 3H) |
| 35 | 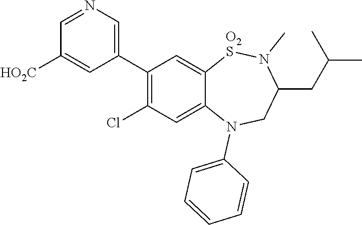 [SMILES: CC(C)CC1CN(c2ccccc2)c2cc(Cl)c(-c3cncc(C(=O)O)c3)cc2S(=O)(=O)N1C] | [M + H]⁺ 500.2 | |
| 36 | 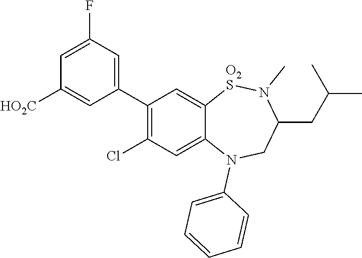 [SMILES: CC(C)CC1CN(c2ccccc2)c2cc(Cl)c(-c3cc(F)cc(C(=O)O)c3)cc2S(=O)(=O)N1C] | [M + H]⁺ 517.2 | |
| 37 | 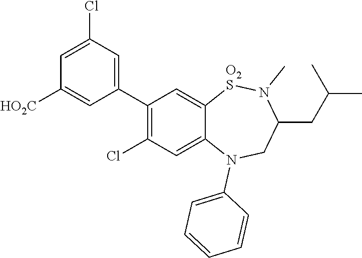 [SMILES: CC(C)CC1CN(c2ccccc2)c2cc(Cl)c(-c3cc(Cl)cc(C(=O)O)c3)cc2S(=O)(=O)N1C] | [M + H]⁺ 533.2 | |
| 38 | 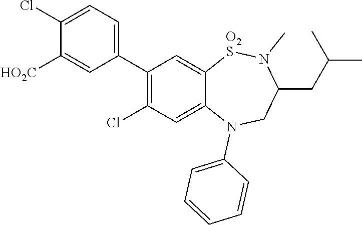 [SMILES: CC(C)CC1CN(c2ccccc2)c2cc(Cl)c(-c3ccc(Cl)c(C(=O)O)c3)cc2S(=O)(=O)N1C] | [M + H]⁺ 533.2 | |
| 39 | 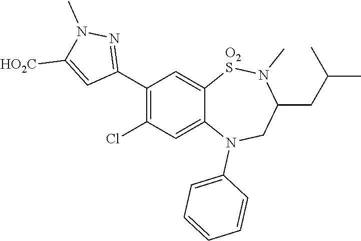 [SMILES: CC(C)CC1CN(c2ccccc2)c2cc(Cl)c(-c3cc(C(=O)O)n(C)n3)cc2S(=O)(=O)N1C] | [M + H]⁺ 503.2 | |
| 40 | 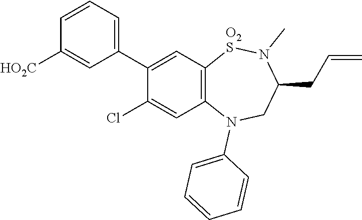 [SMILES: C=CC[C@H]1CN(c2ccccc2)c2cc(Cl)c(-c3cccc(C(=O)O)c3)cc2S(=O)(=O)N1C] | [M + H]⁺ 483.0 | |
| 41 | 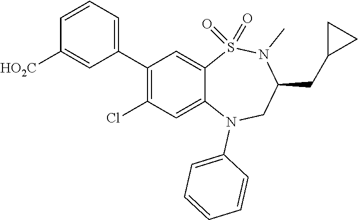 [SMILES: CN1[C@@H](CC2CC2)CN(c2ccccc2)c2cc(Cl)c(-c3cccc(C(=O)O)c3)cc2S1(=O)=O] | [M + H]⁺ 497.2 | |
| 42 | 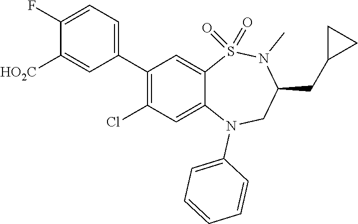 [SMILES: CN1[C@@H](CC2CC2)CN(c2ccccc2)c2cc(Cl)c(-c3ccc(F)c(C(=O)O)c3)cc2S1(=O)=O] | | |
| 43 | 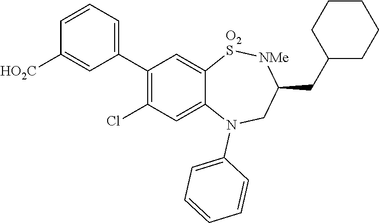 [SMILES: CN1[C@@H](CC2CCCCC2)CN(c2ccccc2)c2cc(Cl)c(-c3cccc(C(=O)O)c3)cc2S1(=O)=O] | [M + H]⁺ 539.2 | |
| 44 | 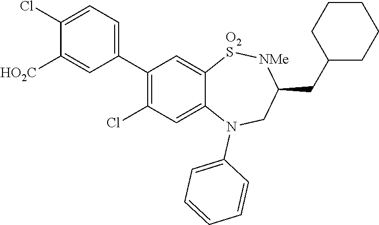 [SMILES: CN1[C@@H](CC2CCCCC2)CN(c2ccccc2)c2cc(Cl)c(-c3ccc(Cl)c(C(=O)O)c3)cc2S1(=O)=O] | [M + H]⁺ 573.2 | |
| 45 | 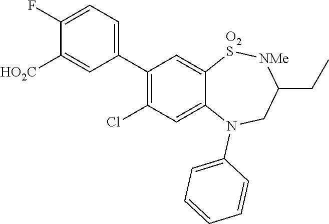 [SMILES: CCC1CN(c2ccccc2)c2cc(Cl)c(-c3ccc(F)c(C(=O)O)c3)cc2S(=O)(=O)N1C] | [M + H]⁺ 489.2 | |
| 46 | 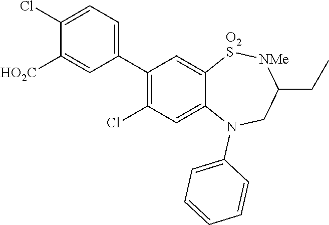 [SMILES: CCC1CN(c2ccccc2)c2cc(Cl)c(-c3ccc(Cl)c(C(=O)O)c3)cc2S(=O)(=O)N1C] | [M + H]⁺ 505.0 | |
| 47 | 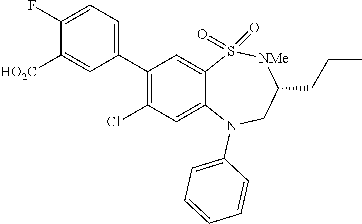 [SMILES: CCC[C@@H]1CN(c2ccccc2)c2cc(Cl)c(-c3ccc(F)c(C(=O)O)c3)cc2S(=O)(=O)N1C] | [M + H]⁺ 503.2 | |
| 48 | 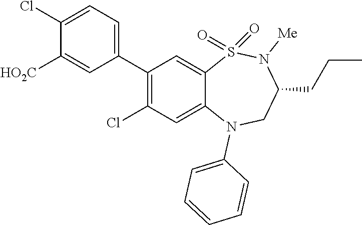 [SMILES: CCC[C@@H]1CN(c2ccccc2)c2cc(Cl)c(-c3ccc(Cl)c(C(=O)O)c3)cc2S(=O)(=O)N1C] | [M + H]⁺ 519.2 | |
| 49 | 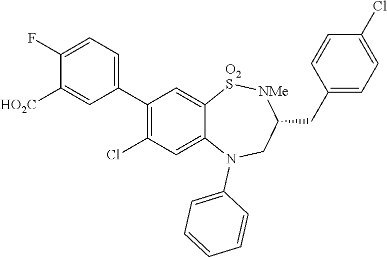 [SMILES: CN1[C@H](Cc2ccc(Cl)cc2)CN(c2ccccc2)c2cc(Cl)c(-c3ccc(F)c(C(=O)O)c3)cc2S1(=O)=O] | [M + H]⁺ 585.2 | |
| 50 | 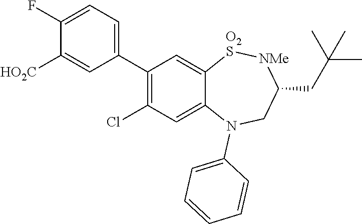 [SMILES: CN1[C@H](CC(C)(C)C)CN(c2ccccc2)c2cc(Cl)c(-c3ccc(F)c(C(=O)O)c3)cc2S1(=O)=O] | [M + H]⁺ 531.2 | |
| 51 | 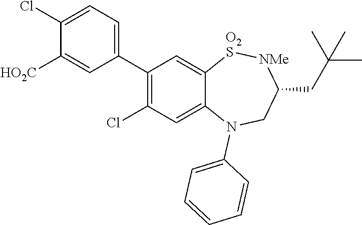 [SMILES: CN1[C@H](CC(C)(C)C)CN(c2ccccc2)c2cc(Cl)c(-c3ccc(Cl)c(C(=O)O)c3)cc2S1(=O)=O] | [M + H]⁺ 547.2 | |
| 52 | 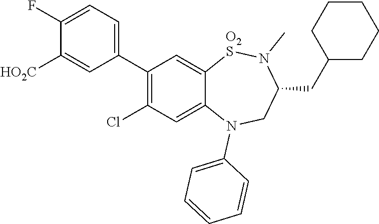 [SMILES: CN1[C@H](CC2CCCCC2)CN(c2ccccc2)c2cc(Cl)c(-c3ccc(F)c(C(=O)O)c3)cc2S1(=O)=O] | [M + H]⁺ 557.2 | |
| 53 | 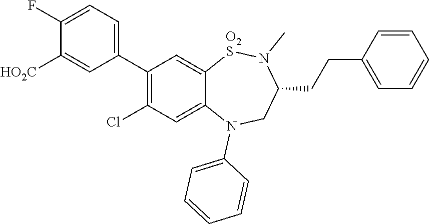 [SMILES: CN1[C@H](CCc2ccccc2)CN(c2ccccc2)c2cc(Cl)c(-c3ccc(F)c(C(=O)O)c3)cc2S1(=O)=O] | [M + H]⁺ 565.0 | |
| 54 | 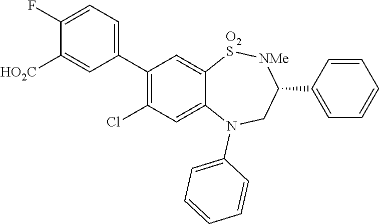 [SMILES: CN1[C@H](c2ccccc2)CN(c2ccccc2)c2cc(Cl)c(-c3ccc(F)c(C(=O)O)c3)cc2S1(=O)=O] | [M + H]⁺ 537.2 | ¹H NMR (400 MHz, Acetonitrile- d₃) δ 8.04 (dd, J = 7.1, 2.4 Hz, 1H), 7.89 (s, 1H), 7.75 (dtd, J = 8.1, 3.4, 1.6 Hz, 1H), 7.53-7.23 (m, 9H), 6.97-6.92 (m, 3H), 5.25-4.98 (m, 1H), 4.60 (dd, J = 15.7, 2.2 Hz, 1H), 4.11 (dd, J = 15.9, 10.9 Hz, 1H), 2.53 (s, 3H) |
| 55 | 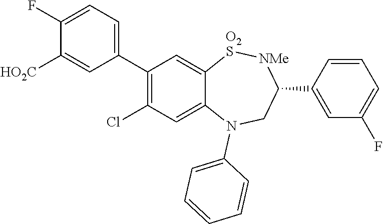 [SMILES: CN1[C@H](c2cccc(F)c2)CN(c2ccccc2)c2cc(Cl)c(-c3ccc(F)c(C(=O)O)c3)cc2S1(=O)=O] | [M + H]⁺ 555.1 | |
| 56 | 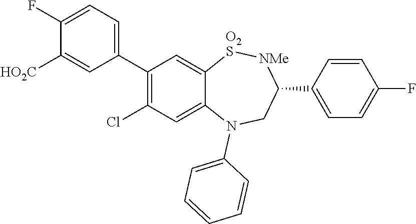 [SMILES: CN1[C@H](c2ccc(F)cc2)CN(c2ccccc2)c2cc(Cl)c(-c3ccc(F)c(C(=O)O)c3)cc2S1(=O)=O] | [M + H]⁺ 555.1 | |
| 57 | 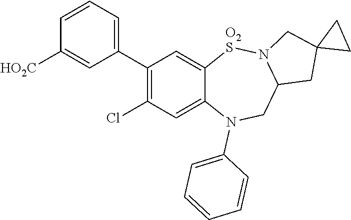 [SMILES: O=C(O)c1cccc(-c2cc3c(cc2Cl)N(c2ccccc2)CC2CC4(CC4)CN2S3(=O)=O)c1] | [M + H]⁺ 495.2 | ¹H NMR (400 MHz, dimethyl- sulfoxide-d₆) δ 13.19 (br s, 1H), 8.03-8.01 (m, 2H), 7.80-7.77 (m, 2H), 7.66-7.62 (m, 1H), 7.50 (s, 1H), 7.30-7.26 (m, 2H), 6.94-6.87 (m, 3H), 4.46 (dd, J = 15.9, 2.2 Hz, 1H), 4.15-4.09 (m, 1H), 3.14 (d, J = 9.5 Hz, 1H), 2.37-2.23 (m, 1H), 1.68-1.60 (m, 1H), 0.73-0.47 (m, 4H) |
| 58 | 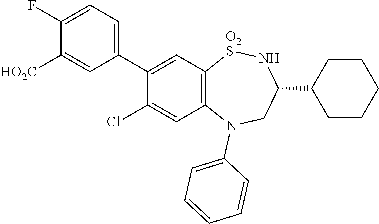 [SMILES: O=C(O)c1cc(-c2cc3c(cc2Cl)N(c2ccccc2)C[C@@H](C2CCCCC2)NS3(=O)=O)ccc1F] | [M + H]⁺ 529.1 | |

### Ex.59: Synthesis of (R)-5-(7-chloro-3-isobutyl-2-methyl-1,1-dioxido-5-phenyl-2,3,4,5-tetrahydrobenzo[f][1,2,5]thiadiazepin-8-yl)-2-fluorobenzoic acid

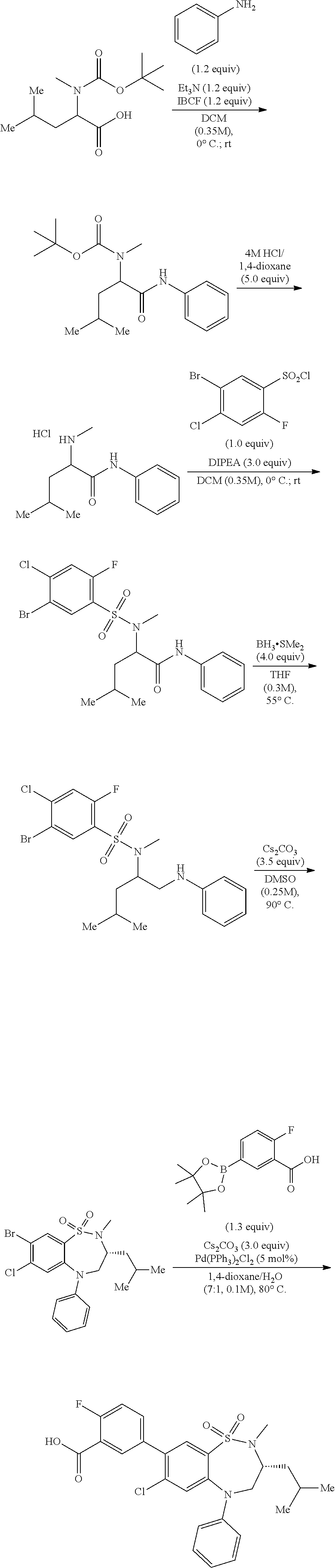

Step 1

In a 40 mL vial equipped with a stir bar, N-(tert-butoxycarbonyl)-N-methyl-D-leucine (1.10 g, 4.48 mmol, 1.0 equiv, CAS #89536-84-5) was dissolved in methylene chloride (12.8 mL, 0.35M). The resulting solution was cooled in an ice bath prior to the addition of isobutyl chloroformate (735.0 mg, 0.71 mL, 5.38 mmol, 1.2 equiv) and triethylamine (544.0 mg, 0.75 mL, 5.38 mmol, 1.2 equiv). The resulting mixture was stirred for 20 minutes before aniline (501.0 mg, 0.49 mL, 5.38 mmol, 1.2 equiv) was added. After stirring overnight, the reaction mixture was concentrated and the residue was purified by silica gel column chromatography (gradient elution, ethyl acetate/cyclohexane, 0 to 20% ethyl acetate) to afford tert-butyl (R)-methyl(4-methyl-1-oxo-1-(phenylamino)pentan-2-yl)carbamate (1.35 g, 94%). ESI MS m/z=265.2 [M-C₄H₈+H]⁺, 319.2 [M−H]⁻.

Step 2

In a 40 mL vial equipped with a stir bar, tert-butyl (R)-methyl(4-methyl-1-oxo-1-(phenylamino)pentan-2-yl)carbamate (1.35 g, 4.23 mmol, 1.0 equiv) was treated with HCl (4M in dioxane, 5.28 mL, 5.0 equiv). The reaction was stirred until LCMS analysis indicated full consumption of the starting material. Upon completion, the reaction was concentrated to afford (R)-4-methyl-2-(methylamino)-N-phenylpentanamide hydrochloride which was used directly without purification in the subsequent step (1.09 g theoretical).

Step 3

In a 40 mL vial equipped with a stir bar, (R)-4-methyl-2-(methylamino)-N-phenylpentanamide hydrochloride (1.09 g theoretical) produced in step 2 was suspended in methylene chloride (12.1 mL, 0.35M). The suspension was cooled in an ice bath and N,N-diisopropylethylamine (3.0 equiv) was added, followed by 5-bromo-4-chloro-2-fluorobenzenesulfonyl chloride (1.30 g, 4.23 mmol, 1.0 equiv). The resulting mixture was stirred overnight while being allowed to warm to room temperature. Upon completion, the reaction mixture was concentrated and purified by silica gel column chromatography (gradient elution, ethyl acetate/cyclohexane, 0 to 30% ethyl acetate) to afford (R)-2-((5-bromo-4-chloro-2-fluoro-N-methylphenyl)sulfonamido)-4-methyl-N-phenylpentanamide (2.08 g). ESI MS m/z=491.0 [M+H]⁺.

Step 4

In a 40 mL vial equipped with a stir bar, (R)-2-((5-bromo-4-chloro-2-fluoro-N-methylphenyl)sulfonamido)-4-methyl-N-phenylpentanamide (2.08 g, 4.23 mmol, 1.0 equiv) was dissolved in tetrahydrofuran (14.1 mL, 0.3M). Next, borane-dimethyl sulfide complex (1.28 g, 1.61 mL, 4.0 equiv) was added. The resulting mixture was heated at 52° C. for 12 h. Upon cooling to room temperature, the reaction was slowly quenched by the addition of 1.0 mL of water. The mixture was concentrated and the residue was purified by silica gel column chromatography (gradient elution, ethyl acetate/cyclohexane, 0 to 30% ethyl acetate) to afford (R)-5-bromo-4-chloro-2-fluoro-N-methyl-N-(4-methyl-1-(phenylamino)pentan-2-yl)benzenesulfonamide (1.60 g, 79%). ESI MS m/z=477.0 [M+H]⁺.

Step 5

In a 40 mL vial equipped with a stir bar, (R)-5-bromo-4-chloro-2-fluoro-N-methyl-N-(4-methyl-1-(phenylamino)pentan-2-yl)benzenesulfonamide (1.60 g, 3.34 mmol, 1.0 equiv) was dissolved in dimethyl sulfoxide (13.4 mL, 0.25M). Cesium carbonate (3.81 g, 11.7 mmol, 3.0 equiv) was added, and the mixture was heated at 90° C. for 6 h. Upon cooling to room temperature, the reaction mixture was concentrated using a Biotage® V-10 evaporator and the residue was purified by silica gel column chromatography (gradient elution, ethyl acetate/cyclohexane, 0 to 15% ethyl acetate) to afford (R)-8-bromo-7-chloro-3-isobutyl-2-methyl-5-phenyl-2,3,4,5-tetrahydrobenzo[f][1,2,5]thiadiazepine 1,1-dioxide (790 mg, 52% yield). ESI MS m/z=457.0 [M+H]⁺.

Step 6

In a 4 mL vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (40.0 mg, 0.087 mmol, 1.0 equiv) was combined neat with 2-fluoro-5-(4,4,5,5-tetramethyl-1,3,2-dioxaborolan-2-yl)benzoic acid (30.2 mg, 0.114 mmol, 1.3 equiv), cesium carbonate (85.0 mg, 0.262 mmol, 3.0 equiv), and bis(triphenylphosphine)palladium( ) chloride (3.1 mg, 5 mol %) under a nitrogen atmosphere. Next, 1,4-dioxane (0.76 mL) and water (0.11 mL) were added, and the vial was sealed with electrical tape and heated at 80° C. for 40 min. Upon cooling to room temperature, the reaction mixture was quenched by the addition of formic acid (0.25 mL) and concentrated. The residue was purified by RPHIPLC to afford the product, which was lyophilized from mixture of acetonitrile and water to afford (R)-5-(7-chloro-3-isobutyl-2-methyl-1,1-dioxido-5-phenyl-2,3,4,5-tetrahydrobenzo[f][1,2,5]thiadiazepin-8-yl)-2-fluorobenzoic acid (11.4 mg, 25%). ESI MS m/z=517.2 [M+H]⁺. ¹H NMR (400 MHz, acetone-d₆ δ 8.09 (dd, J 6.9, 2.5 Hz, 1H), 7.92 (s, 1H), 7.83 (ddd, J 8.6, 4.5, 2.5 Hz, 1H), 7.42 (dd, J=10.6, 8.5 Hz, 1H), 7.36-7.20 (m, 3H), 7.11-6.91 (m, 3H), 4.18 (d, J=15.9 Hz, 1H), 4.04 (s, 1H), 3.69 (s, 1H), 2.75 (s, 3H), 1.86-1.55 (in, 2H), (ddd, J=14.3, 9.2, 5.3 Hz, 1H), 0.97-0.93 (m, 6H).

Example 60 was prepared using a procedure analogous to that used above for Ex. 59

| Example | Structure | MS | NMR |
|---|---|---|---|
| 59 | 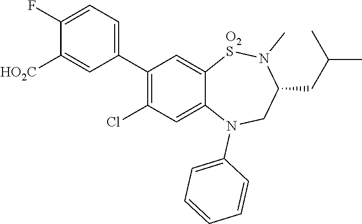 [SMILES: CC(C)C[C@@H]1CN(c2ccccc2)c2cc(Cl)c(-c3ccc(F)c(C(=O)O)c3)cc2S(=O)(=O)N1C] | [M + H]⁺ 517.2 | ¹H NMR (400 MHz, acetone-d6 δ 8.09 (dd, J =6.9, 2.5 Hz, 1H), 7.92 (s, 1H), 7.83 (ddd, J = 8.6, 4.5, 2.5 Hz, 1H), 7.42 (dd, J = 10.6, 8.5 Hz, 1H), 7.36-7.20 (m, 3H), 7.11-6.91 (m, 3H), 4.18 (d, J = 15.9 Hz, 1H), 4.04 (s, 1H), 3.69 (s, 1H), 2.75 (s, 3H), 1.86- 1.55 (m, 2H), (ddd, J = 14.3, 9.2, 5.3 Hz, 1H), 0.97-0.93 (m, 6H) |
| 60 | 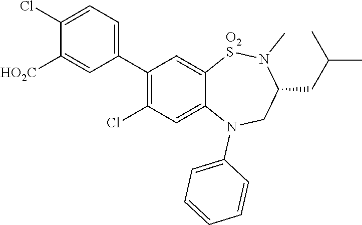 [SMILES: CC(C)C[C@@H]1CN(c2ccccc2)c2cc(Cl)c(-c3ccc(Cl)c(C(=O)O)c3)cc2S(=O)(=O)N1C] | [M + H]⁺ 533.2 | |

### Ex.61: Synthesis of (R)-5-(7-chloro-3-isopropyl-2-methyl-1,1-dioxido-5-phenyl-2,3,4,5-tetrahydrobenzo[f][1,2,5]thiadiazepin-8-y)-2-fluorobenzoic acid

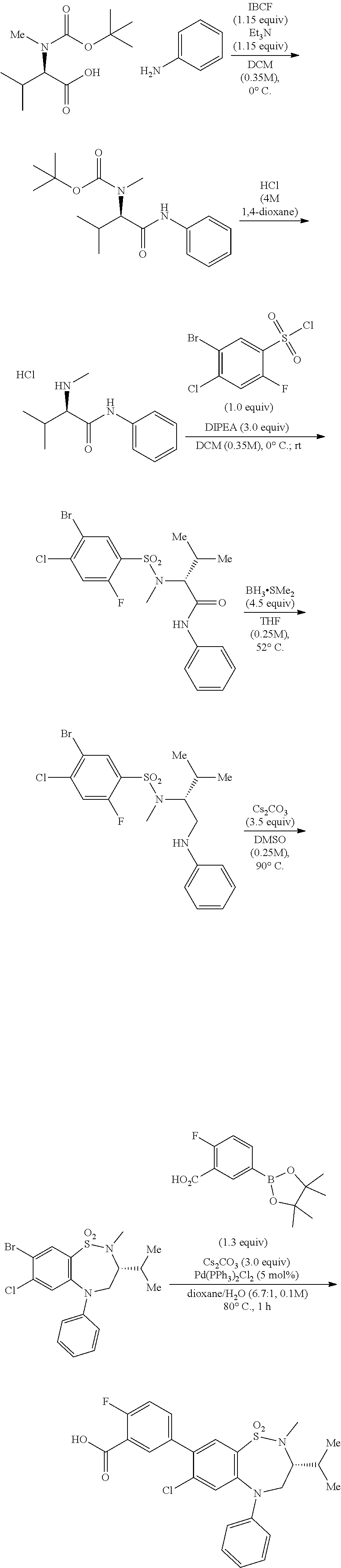

Step 1

In a 40 mL vial equipped with a stir bar, N-(tert-butoxycarbonyl)-N-methyl-D-valine (1.00 g, 4.32 mmol, 1.0 equiv) was dissolved in methylene chloride (12.4 nL, 035M). The resulting solution was cooled in an ice bath, and isobutyl chloroformate (0.653 nL, 679.0 mg, 4.97 mmol, 1.15 equiv) was added, followed immediately by trietylamine (0.693 mL, 503 mg, 4.97 mmol, 1.15 equiv). The resulting mixture was stirred at 0° C. for 15 to 20 min prior to the addition of aniline (0.454 mL, 463 mg, 4.97 mmol, 1.15 equiv). The reaction mixture was stirred for 16 h while being allowed to warm to room temperature and concentrated. Purification of the crude residue by silica gel column chromatography (ethyl acetate/cyclohexane) afforded tert-butyl (R)-methyl(3-methyl-1-oxo-1-(phenylamino)butan-2-ylcarbamate as a white solid in quantitative yield (1.325 g) ESI MS n/z=207.0 [M+H].

Step 2

In a 40 mL vial equipped with a stir bar, tert-butyl (R)-methyl(3-methyl-1-oxo-1-(phenylamino)butan-2-yl)carbonate (1.325 g) was dissolved in 4M HCl in dioxane (5.41 mL, 5.0 equiv). The resulting mixture was stirred for 3 h at room temperature and concentrated to afford (R)-3-methyl-2-(methylamino)-N-phenylbutanamide hydrochloride (946.1 tug, 90% yield) as a white solid that was used directly in the subsequent step without purification. ESI MS m/z=207.0 [M+H]⁺.

Step 3

In a 40 mL vial equipped with a stir bar (R)-3-methyl-2-(methylamino)-N-phenyl butanamide hydrochloride (946.1 mg, 3.90 mmol, 1.0 equiv) was suspended in methylene chloride (11.1 ml, 0.35M). The resulting suspension was cooled to 0° C. prior to the addition of N,N-diisopropylethylamine (2.04 ml, 1.51 g, 11.69 mmol, 3.0 equiv). The resulting homogenous mixture was charged with 5-bromo-4-chloro-2-fluorobenzenesulfonyl chloride (1.20 g, 3.90 mmol, L 0 equiv) and was stirred for 16 h while being allowed to warm to room temperature. Upon full conversion, as judged by LCMS analysis of the reaction mixture, the reaction was concentrated and purified by silica gel column chromatography (gradient elution, 0 to 30% ethyl acetate in cyclohexane) to afford (R)-2-((5-bromo-4-chloro-2-fluoro-N-methylphenyl)sulfonamido)-3-methyl-N-phenylbutanamide (1.51 g, 81% yield). ESI MS m/z=477.0 [M+H]⁺.

Step 4

In a 12 mL vial equipped with a stir bar (R)-2-((5-bromo-4-chloro-2-fluoro-N-methylphenyl)sulfonamido)-3-methyl-N-phenylbutanamide (1.51 g, 3.16 mmol, 1.0 equiv) was dissolved in tetrahydrofuran (12.6 mL, 0.25M) under a nitrogen atmosphere. Next, borane dimethyl sulfide complex (1.35 mL, 4.5 equiv) was added, and the resulting mixture was heated at 52° C. for 24 h. Upon cooling to room temperature, the reaction mixture was carefully quenched with 1.0 mL of water and concentrated. Purification by silica gel column chromatography (gradient elution, ethyl acetate in cyclohexane, 0 to 40%) afforded (R)-5-bromo-4-chloro-2-fluoro-N-methyl-N-(3-methyl-1-(phenylamino)butan-2-yl)benzenesulfonamide (1.06 g, 72% yield). ESI MS m/z=463.0 [M+H]⁻.

Step 5

In a 40 mL vial equipped with a stir bar, (R)-5-bromo-4-chloro-2-fluoro-N-methyl-N-(3-methyl-1-(phenylamino)butan-2-yl)benzenesulfonamide (1.06 g, 2.28 mmol, 1.0 equiv) was dissolved in DMSO (9.13 ml, 0.25M). Cesium carbonate (2.60 g, 7.99 mmol, 3.5 equiv) was added, and the reaction mixture was heated at 90° C. for 45 min. Upon cooling to room temperature, the crude reaction mixture was concentrated on a Biotage® V-10 evaporator, and the soluble residue was purified by silica gel column chromatography (10% ethyl acetate/cyclohexane) to afford (R)-8-bromo-7-chloro-3-isopropyl-2-methyl-5-phenyl-2,3,4,5-tetrahydrobenzo[f][1,2,5]thiadiazepine 1,1-dioxide (648.3 mg, 1.46 mmol, 64% yield). ESI MS m/z=443.0 [M+H]⁺.

Step 6

In a 4 mL vial equipped with a stir bar equipped with a stir bar, (R)-8-bromo-7-chloro-3-isopropyl-2-methyl-5-phenyl-2,3,4,5-tetrahydrobenzo[f][1,2,5]thiadiazepine 1,1-dioxide (30 mg, 0.068 mmol, 1.0 equiv), cesium carbonate (66.1 mg, 0.203 mmol, 3.0 equiv), 2-fluoro-5-(4,4,5,5-tetramethyl-1,3,2-dioxaborolan-2-yl)benzoic acid (23.38 mg, 0.088 mmol, 1.3 equiv, CAS #: 867256-77-7), and bis(triphenylphosphine)palladium(II) chloride (2.37 mg, 5 mol %, CAS #: 13965-03-2) were combined neat under a nitrogen atmosphere. Next, 1,4-dioxane (0.59 mL) and water (0.9 mL) were added, and the reaction mixture was heated at 80° C. for 40 min. Upon cooling to room temperature, the reaction mixture was quenched with 250 μL of formic acid, passed through a 0.45 micron syringe filter using N,N-dimethylformamide to rinse, and purified by RPHPLC to afford a white solid after concentration that was lyophilized from a mixture of acetonitrile and water to afford (R)-5-(7-chloro-3-isopropyl-2-methyl-1,1-dioxido-5-phenyl-2,3,4,5-tetrahydrobenzo[f][1,2,5]thiadiazepin-8-yl)-2-fluorobenzoic acid (18.6 mg, 0.037 mmol, 55% yield). ESI MS m/z=503.0 [M+H]⁺. ¹H NMR (500 MHz, acetonitrile-d3) δ 8.02 (dd, J=6.9, 2.5 Hz, 1H), 7.84 (s, 1H), 7.74 (ddd, J=8.6, 4.6, 2.5 Hz, 1H), 7.38-7.30 (m, 3H), 7.27 (s, 1H), 7.03-7.00 (m, 3H), 4.34 (d, J=16.1 Hz, 1H), 3.72 (s, 1H), 3.34 (t, J=10.6 Hz, 1H), 2.75 (s, 3H), 1.08 (d, J=6.6 Hz, 3H), 1.06 (d, J=6.6 Hz, 3H).

Examples 62 and 63 were prepared using a procedure analogous to that used above for Ex.61

| Example | Structure | MS | NMR |
|---|---|---|---|
| 61 | 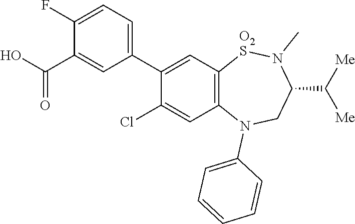 [SMILES: CC(C)[C@@H]1CN(c2ccccc2)c2cc(Cl)c(-c3ccc(F)c(C(=O)O)c3)cc2S(=O)(=O)N1C] | [M + H]⁺ 503.0 | ¹H NMR (500 MHz, acetonitrile- d₃) δ 8.02 (dd, J = 6.9, 2.5 Hz, 1H), 7.84 (s, 1H), 7.74 (ddd, J = 8.6, 4.6, 2.5 Hz, 1H), 7.38-7.30 (m, 3H), 7.27 (s, 1H), 7.03-7.00 (m, 3H), 4.34 (d, J = 16.1 Hz, 1H), 3.72 (s, 1H), 3.34 (t, J = 10.6 Hz, 1H), 2.75 (s, 3H), 2.09 (m, 1H), 1.08 (d, J = 6.6 Hz, 3H), 1.06 (d, J = 6.6 Hz, 3H) |
| 62 | 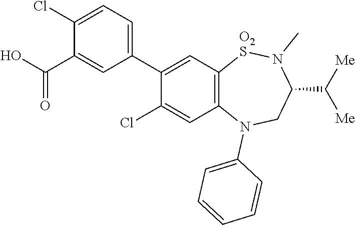 [SMILES: CC(C)[C@@H]1CN(c2ccccc2)c2cc(Cl)c(-c3ccc(Cl)c(C(=O)O)c3)cc2S(=O)(=O)N1C] | [M + H]⁺ 519.1 | ¹H NMR (500 MHz, acetonitrile- d₃)δ 7.94 (dd, J = 1.8, 0.9 Hz, 1H), 7.85 (s, 1H), 7.65-7.61 (m, 2H), 7.35 (t, J = 7.9 Hz, 2H), 7.26 (s, 1H), 7.04-7.01 (m, 3H), 4.34 (d, J = 16.1 Hz, 1H), 3.74 (s, 1H), 3.33 (t, J = 10.7 Hz, 1H), 2.09 (m, 1H), 1.08-1.05 (m, 6H) |
| 63 | 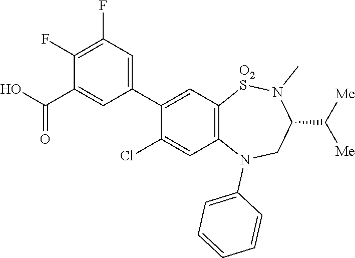 [SMILES: CC(C)[C@@H]1CN(c2ccccc2)c2cc(Cl)c(-c3cc(F)c(F)c(C(=O)O)c3)cc2S(=O)(=O)N1C] | [M + H]⁺ 521.1 | |

### Ex.64: Synthesis of(R)-5-(7-chloro-3-cyclopropyl-2-methyl-1,1-dioxido-5-phenyl-2,3,4,5-tetrahydrobenzo[f][1,2,5]thiadiazepin-8-yl)-2-fluorobenzoic acid

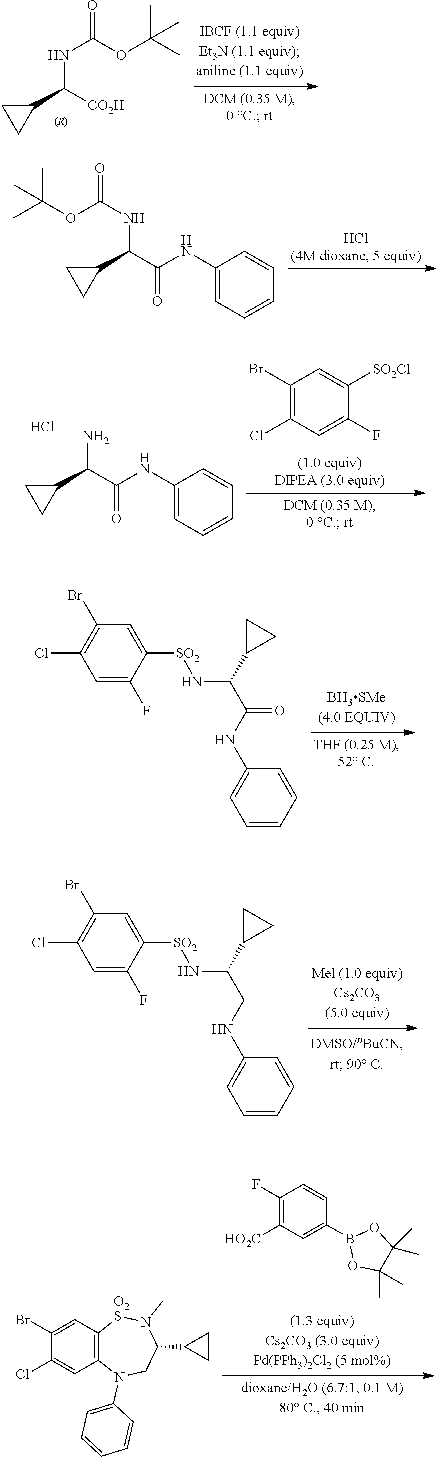

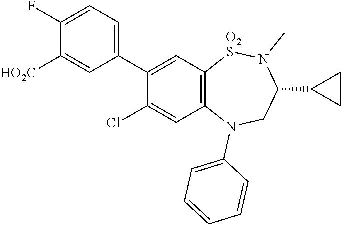 [SMILES: CN1[C@H](C2CC2)CN(c2ccccc2)c2cc(Cl)c(-c3ccc(F)c(C(=O)O)c3)cc2S1(=O)=O]

Step 1

In a 40 mL vial equipped with a stir bar, (R)-2-((tert-butoxycarbonyl)amino)-2-cyclopropylacetic acid (1.00 g, CAS: 609768-49-2) was dissolved in DCM (13.3 mL, 0.35M). The solution was cooled in an ice and water bath prior to the successive addition of isobutyl chloroformate (670 μL, 1.1 equiv) and triethylamine (710 μL, 1.1 equiv). The resulting mixture was stirred for 30 minutes prior to the addition of aniline (470 μL, 1.1 equiv). The reaction was stirred a further 16 h while being allowed to warm to room temperature. Upon completion, the reaction mixture was concentrated, and the crude material was purified by silica gel column chromatography to afford tert-butyl (R)-(1-cyclopropyl-2-oxo-2-(phenylamino)ethyl)carbamate (1.30 g, 97%). ESI MS m/z=313.2 [M+Na]*.

Step 2

In a 40 mL vial equipped with a stir bar, tert-butyl (R)-(1-cyclopropyl-2-oxo-2-(phenylamino)ethyl)carbamate (1.30 g) was combined with hydrochloric acid (4M in dioxane, 5.6 mL. 5 equiv). The mixture was stirred for 3 h and concentrated to afford (R)-2-amino-2-cyclopropyl-N-phenylacetamide hydrochloride (1.02 g, theoretical mass) which was used without purification. ESI MS m/z=191.0 [M+H]⁺.

Step 3

In a 40 mL vial equipped with a stir bar, (R)-2-amino-2-cyclopropyl-N-phenylacetamide hydrochloride (1.02 g) was suspended in dichloromethane (12.8 mL, 0.35M). The suspension was cooled in an ice and water bath and N,N-diisopropylethylamine (2.35 mL, 3.0 equiv) was added followed by 5-bromo-4-chloro-2-fluorobenzenesulfonyl chloride (1.38 g, 1.0 equiv, CAS #: 1070972-67-6). The resulting solution was stirred for 3 h and concentrated. The crude residue was purified by silica gel column chromatography to afford (R)-2-((5-bromo-4-chloro-2-fluorophenyl)sulfonamido)-2-cyclopropyl-N-phenylacetamide (1.97 g, 95%). ESI MS m/z=461.0 [M+H]⁺.

Step 4

In a 40 mL vial equipped with a stir bar, (R)-2-((5-bromo-4-chloro-2-fluorophenyl)sulfonamido)-2-cyclopropyl-N-phenylacetamide (1.97 g) was dissolved in tetrahydrofuran (13.9 mL, 0.3M) under a nitrogen atmosphere. Borane-dimethyl sulfide complex (1.59 mL, 4.0 equiv) was added, and the mixture was heated at 52° C. for 24 h. Upon cooling to room temperature, the reaction was slowly quenched with water (1.0 mL) and concentrated. The crude residue was purified by silica gel column chromatography to afford (R)-5-bromo-4-chloro-N-(1-cyclopropyl-2-(phenylamino)ethyl)-2-fluorobenzenesulfonamide (1.68 g, 90%). ESI MS m/z=447.0 [M+H]⁺.

Step 5

Dimethyl sulfoxide (15.0 mL, 0.25M) was added to a 40 mL vial containing a stir bar and (R)-5-bromo-4-chloro-N-(1-cyclopropyl-2-(phenylamino)ethyl)-2-fluorobenzenesulfonamide (1.68 g). Cesium carbonate (6.11 g, 5.0 equiv) was added, followed by a solution of iodomethane (3M in butyronitrile, 1.25 mL, 1.0 equiv). Within 15 minutes, LCMS had indicated full conversion to the N-methylsulfonamide and the mixture was subsequently heated at 90° C. for 14 h. Upon cooling to room temperature, the reaction mixture was concentrated using a Biotage® V-10 evaporator and the crude residue was purified by silica gel column chromatography to afford (R)-8-bromo-7-chloro-3-cyclopropyl-2-methyl-5-phenyl-2,3,4,5-tetrahydrobenzo[f][1,2,5]thiadiazepine 1,1-dioxide (1.09 g, 66% yield). ESI MS m/z=440.8 [M+H]⁺.

Step 6

Cesium carbonate (89.0 mg, 3.0 equiv), 2-fluoro-5-(4,4,5,5-tetramethyl-1,3,2-dioxaborolan-2-yl)benzoic acid (31.3 mg, 1.3 equiv, CAS #: 867256-77-7), bis(triphenylphosphine)palladium(II) chloride (3.2 mg, 5 mol %, CAS #: 13965-03-2), and (R)-8-bromo-7-chloro-3-cyclopropyl-2-methyl-5-phenyl-2,3,4,5-tetrahydrobenzo[f][1,2,5]thiadiazepine 1,1-dioxide (1.09 g, 66% yield) were combined neat in a 4 mL vial equipped with a stir bar under a nitrogen atmosphere. Dioxane (790 μL) and water (120 μL) were added, and the vial was sealed with electrical tape. The reaction was heated at 80° C. for 30 min. Upon cooling to room temperature, the reaction was quenched with formic acid (400 μL) and concentrated. The crude residue was re-dissolved in N,N-dimethylformamide, passed through a 0.2 μm syringe filter, and purified by RPHPLC to afford (R)-5-(7-chloro-3-cyclopropyl-2-methyl-1,1-dioxido-5-phenyl-2,3,4,5-tetrahydrobenzo[f][1,2,5]thiadiazepin-8-yl)-2-fluorobenzoic acid (8.3 mg, 18% yield). ESI MS m/z=501.2 [M+H]⁺. ¹H NMR (400 MHz, acetonitrile-d₃) δ 8.03 (dd, J=6.9, 2.4 Hz, 1H), 7.87 (s, 1H), 7.75 (ddd, J=8.7, 4.6, 2.5 Hz, 1H), 7.42 (s, 1H), 7.34 (dd, J=10.8, 8.6 Hz, 1H), 7.27 (dd, J=8.7, 7.2 Hz, 2H), 7.01-6.87 (m, 1H), 6.84 (d, J=8.1 Hz, 2H), 4.26 (d, J=16.1 Hz, 1H), 3.64-3.51 (m, 1H), 3.05 (m, 1H), 2.78 (s, 3H), 1.03-0.94 (m, 1H), 0.82-0.58 (m, 2H), 0.50-0.28 (m, 2H).

Examples 65-79 were prepared by using similar procedures to that described above for Ex. 64:

| Example | Structure | MS | NMR |
|---|---|---|---|
| 64 | 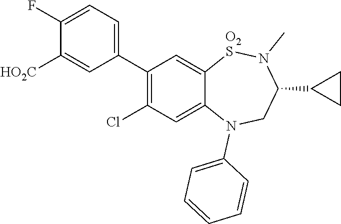 [SMILES: CN1[C@H](C2CC2)CN(c2ccccc2)c2cc(Cl)c(-c3ccc(F)c(C(=O)O)c3)cc2S1(=O)=O] | [M + H]⁺ 501.2 | ¹H NMR (400 MHz, acetonitrile-d₃) δ 8.03 (dd, J = 6.9, 2.4 Hz, 1H), 7.87 (s, 1H), 7.75 (ddd, J = 8.7, 4.6, 2.5 Hz, 1H), 7.42 (s, 1H), 7.34 (dd, J = 10.8, 8.6 Hz, 1H), 7.27 (dd, J = 8.7, 7.2 Hz, 2H), 7.01-6.87 (m, 1H), 6.84 (d, J = 8.1 Hz, 2H), 4.26 (d, J = 16.1 Hz, 1H), 3.64-3.51 (m, 1H), 3.05 (m, 1H), 2.78 (s, 3H), 1.03-0.94 (m, 1H), 0.82-0.58 (m, 2H), 0.50-0.28 (m, 2H). |
| 65 | 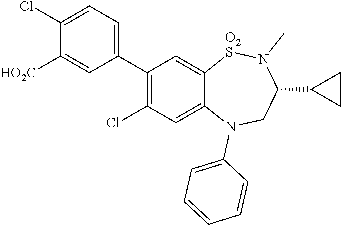 [SMILES: CN1[C@H](C2CC2)CN(c2ccccc2)c2cc(Cl)c(-c3ccc(Cl)c(C(=O)O)c3)cc2S1(=O)=O] | [M + H]⁺ 517.2 | |
| 66 | 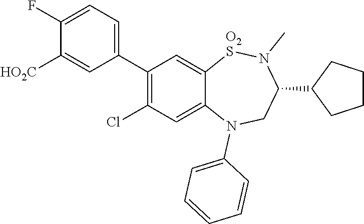 [SMILES: CN1[C@H](C2CCCC2)CN(c2ccccc2)c2cc(Cl)c(-c3ccc(F)c(C(=O)O)c3)cc2S1(=O)=O] | [M + H]⁺ 529.2 | |
| 67 | 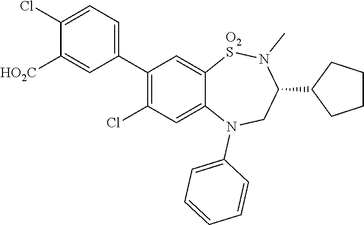 [SMILES: CN1[C@H](C2CCCC2)CN(c2ccccc2)c2cc(Cl)c(-c3ccc(Cl)c(C(=O)O)c3)cc2S1(=O)=O] | [M + H]⁺ 545.2 | |
| 68 | 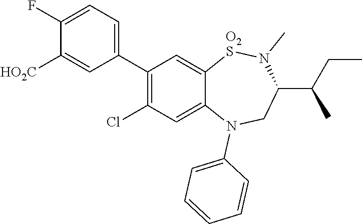 [SMILES: CC[C@@H](C)[C@@H]1CN(c2ccccc2)c2cc(Cl)c(-c3ccc(F)c(C(=O)O)c3)cc2S(=O)(=O)N1C] | [M + H]⁺ 517.2 | |
| 69 | 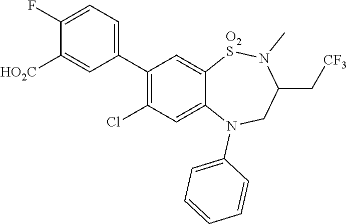 [SMILES: CN1C(CC(F)(F)F)CN(c2ccccc2)c2cc(Cl)c(-c3ccc(F)c(C(=O)O)c3)cc2S1(=O)=O] | [M + H]⁺ 543.2 | |
| 70 | 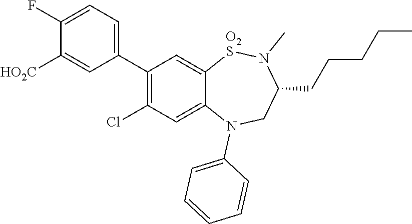 [SMILES: CCCCC[C@@H]1CN(c2ccccc2)c2cc(Cl)c(-c3ccc(F)c(C(=O)O)c3)cc2S(=O)(=O)N1C] | [M + H]⁺ 531.2 | |
| 71 | 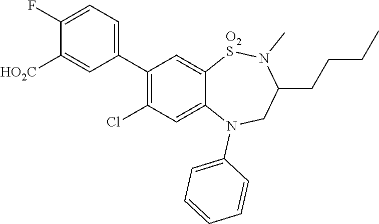 [SMILES: CCCCC1CN(c2ccccc2)c2cc(Cl)c(-c3ccc(F)c(C(=O)O)c3)cc2S(=O)(=O)N1C] | [M + H]⁺ 517.1 | |
| 72 | 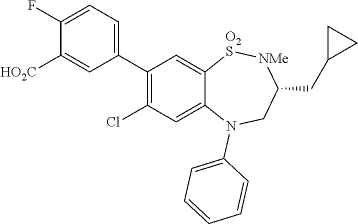 [SMILES: CN1[C@H](CC2CC2)CN(c2ccccc2)c2cc(Cl)c(-c3ccc(F)c(C(=O)O)c3)cc2S1(=O)=O] | [M + H]⁺ 531.2 | ¹H NMR (400 MHz, acetonitrile-d₃) δ 7.94 (app s, 1H), 7.86 (s, 1H), 7.64-7.60 (m, 2H), 7.37-7.23 (m, 3H), 6.98-6.95 (m, 3H), 4.19 (d, J = 15.9 Hz, 1H), 3.95-3.88 (m, 1H), 3.61-3.55 (m, 1H), 2.71 (s, 3H), 1.81-1.74 (m, 1H), 1.28 (dt, J = 14.6, 7.7 Hz, 1H), 0.91-0.80 (m, 1H), 0.62-0.38 (m, 2H), 0.19-0.12 (m, 2H) |
| 73 | 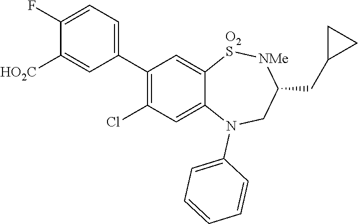 [SMILES: CN1[C@H](CC2CC2)CN(c2ccccc2)c2cc(Cl)c(-c3ccc(F)c(C(=O)O)c3)cc2S1(=O)=O] | [M + H]⁺ 515.2 | ¹H NMR (400 MHz, acetonitrile-d₃) δ 8.02 (dd, J = 6.9, 2.5 Hz, 1H), 7.85 (s, 1H), 7.73 (ddd, J = 8.7, 4.5, 2.4 Hz, 1H), 7.43-7.22 (m, 4H), 7.02-6.77 (m, 3H), 4.19 (d, J = 16.0 Hz, 1H), 3.96-3.90 (m, 1H), 3.59- 3.52 (m, 1H), 2.71 (s, 3H), 1.80- 1.74 (m, 1H), 1.29 (dt, J = 14.6, 7.7 Hz, 1H), 0.93-0.76 (m, 1H), 0.58- 0.51 (m, 2H), 0.19-0.13 (m, 2H) |
| 74 | 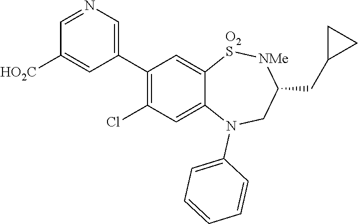 [SMILES: CN1[C@H](CC2CC2)CN(c2ccccc2)c2cc(Cl)c(-c3cncc(C(=O)O)c3)cc2S1(=O)=O] | [M + H]⁺ 498.2 | |
| 75 | 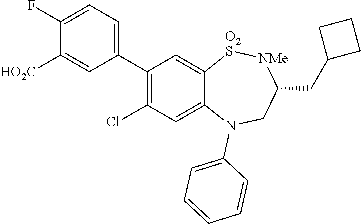 [SMILES: CN1[C@H](CC2CCC2)CN(c2ccccc2)c2cc(Cl)c(-c3ccc(F)c(C(=O)O)c3)cc2S1(=O)=O] | [M + H]⁺ 529.0 | |
| 76 | 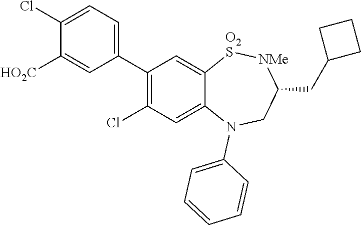 [SMILES: CN1[C@H](CC2CCC2)CN(c2ccccc2)c2cc(Cl)c(-c3ccc(Cl)c(C(=O)O)c3)cc2S1(=O)=O] | [M + H]⁺ 545.2 | |
| 77 | 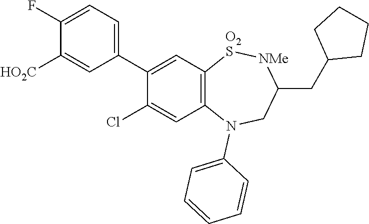 [SMILES: CN1C(CC2CCCC2)CN(c2ccccc2)c2cc(Cl)c(-c3ccc(F)c(C(=O)O)c3)cc2S1(=O)=O] | [M + H]⁺ 543.0 | |
| 78 | 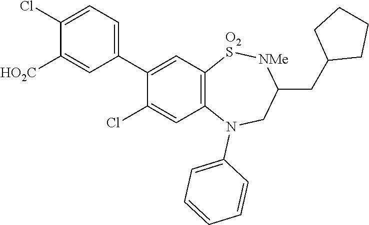 [SMILES: CN1C(CC2CCCC2)CN(c2ccccc2)c2cc(Cl)c(-c3ccc(Cl)c(C(=O)O)c3)cc2S1(=O)=O] | [M + H]⁺ 559.0 | |
| 79 | 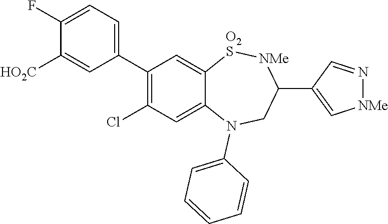 [SMILES: CN1C(c2cnn(C)c2)CN(c2ccccc2)c2cc(Cl)c(-c3ccc(F)c(C(=O)O)c3)cc2S1(=O)=O] | [M + H]⁺ 541.2 | |

### Ex.80: Synthesis of(R)-5-(7-chloro-3-cyclobutyl-2-methyl-1,1-dioxido-5-phenyl-2,3,4,5-tetrahydrobenzo[f][1,2,5]thiadiazepin-8-yl)-2-fluorobenzoic acid

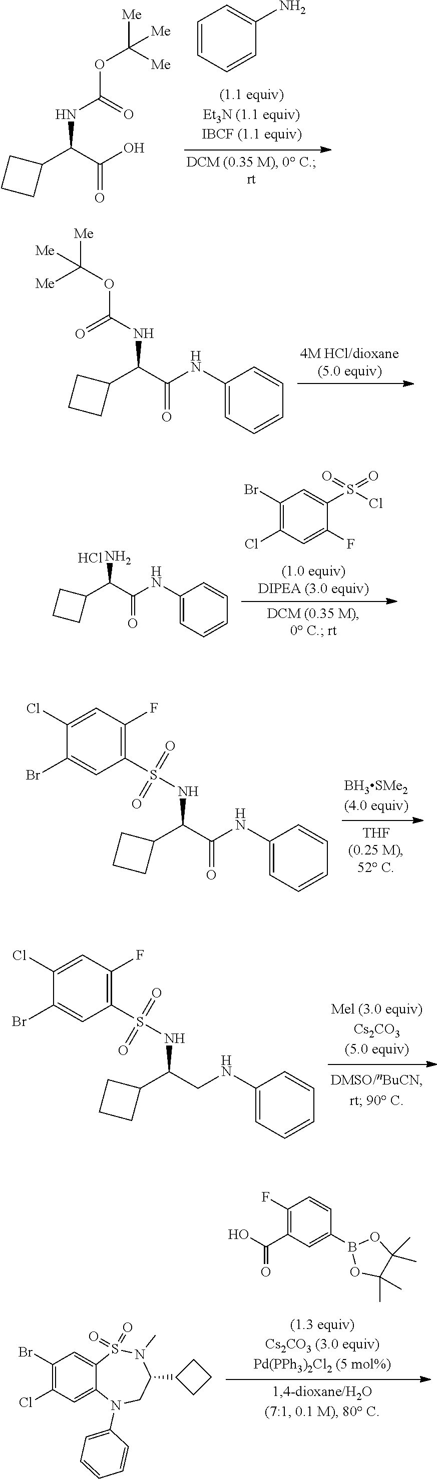

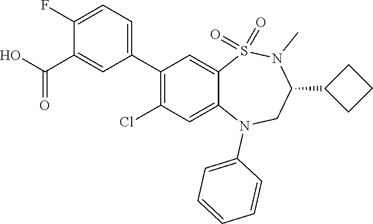 [SMILES: CN1[C@H](C2CCC2)CN(c2ccccc2)c2cc(Cl)c(-c3ccc(F)c(C(=O)O)c3)cc2S1(=O)=O]

Step 1″

In a 40 mL vial equipped with a stir bar, (R)-2-((tert-butoxycarbonyl)amino)-2-cyclo butylacetic acid (1.0 g, 4.36 mmol, 1.0 equiv, CAS 155905-78-5) was dissolved in methylene chloride (12.5 mL, 0.35M). The resulting mixture was cooled in an ice bath. Next, isobutyl chloroformate (655.0 mg, 0.63 mL, 4.80 mmol, 1.1 equiv) and triethylamine (485.0 mg, 0.67 mL, 4.8 mmol, 1.1 equiv) were added. The resulting mixture was stirred for 20 min prior to the addition of aniline (447 mg, 0.44 mL, 4.80 mmol, 1.1 equiv). The reaction was stirred for 7 h while being allowed to warm to room temperature. Upon completion, the reaction mixture was concentrated and purified by silica gel column chromatography (gradient elution, ethyl acetate/cyclohexane, 0 to 30% ethyl acetate) to afford tert-butyl (R)-(1-cyclobutyl-2-oxo-2-(phenylamino)ethyl)carbamate (1.31 g, 99%). ESI MS m/z=249.0 [M-C₄H₈+H]⁺, 327.2 [M+Na]⁺.

Step 2

In a 40 mL vial equipped with a stir bar, tert-butyl (R)-(1-cyclobutyl-2-oxo-2-(phenylamino)ethyl)carbamate (1.31 g, 4.32 mmol, 1.0 equiv) was treated with 4M HCl in 1,4-dioxane (5.40 mL, 5.0 equiv). The resulting mixture was stirred at room temperature for 2 h and concentrated to afford (R)-2-amino-2-cyclobutyl-N-phenylacetamide hydrochloride which was used in the next step without purification (1.04 g theoretical). ESI MS m/z=205.2 [M+H]⁺.

Step 3

In a 40 mL vial equipped with a stir bar, (R)-2-amino-2-cyclobutyl-N-phenylacetamide hydrochloride (1.04 g theoretical) formed in step 3 was suspended in methylene chloride (12.3 mL, 0.35M). The resulting mixture was cooled in an ice bath and N,N-diisopropylethylamine (1.67 g, 2.26 mL, 3.0 equiv) was added. Next, 5-bromo-4-chloro-2-fluorobenzenesulfonyl chloride (1.33 g, 4.32 mmol, 1.0 equiv, CAS #1070972-67-6) was added, and the mixture was stirred overnight while being allowed to warm to room temperature. Upon completion, as determined by LCMS analysis, the reaction mixture was concentrated, and the residue was purified by silica gel column chromatography (gradient elution, ethyl acetate/cyclohexane, 0 to 25% ethyl acetate) to afford (R)-2-((5-bromo-4-chloro-2-fluorophenyl)sulfonamido)-2-cyclobutyl-N-phenylacetamide (1.58 g, 77%). ESI MS m/z=475.0 [M+H]⁺.

Step 4

In a 40 mL vial equipped with a stir bar, (R)-2-((5-bromo-4-chloro-2-fluorophenyl)sulfonamido)-2-cyclobutyl-N-phenylacetamide (1.58 g, 3.31 mmol, 1.0 equiv) was dissolved in tetrahydrofuran (13.3 mL, 0.25M). Next, borane-dimethyl sulfide complex (1.26 mL, 4.0 equiv) was added, and the mixture was heated at 52° C. for 24 h. Upon cooling to room temperature, the reaction mixture was slowly quenched with water (1.0 mL). The crude mixture was concentrated and the residue was purified by silica gel column chromatography (gradient elution, ethyl acetate/cyclohexane, 0 to 40% ethyl acetate) to afford (R)-5-bromo-4-chloro-N-(1-cyclobutyl-2-(phenylamino)ethyl)-2-fluorobenzenesulfonamide (1.52 g). ESI MS m/z=461.0 [M+H]⁺.

Step 5

In a 40 mL vial equipped with a stir bar, (R)-5-bromo-4-chloro-N-(1-cyclobutyl-2-(phenylamino)ethyl)-2-fluorobenzenesulfonamide (1.52 g, 3.29 mmol, 1.0 equiv) was dissolved in dimethyl sulfoxide (13.1 mL, 0.25M). Cesium carbonate (5.35 g, 16.4 mmol, 5.0 equiv) was added, followed by iodomethane as a 3M solution in butyronitrile (1.0 equiv, 1.10 mL). The mixture was stirred for 50 min at room temperature while monitoring for formation of the tertiary sulfonamide, then heated at 90° C. for 17 h. Upon cooling to room temperature, the reaction mixture was concentrated using a Biotage® V-10 evaporator. Purification of the residue by silica gel column chromatography afforded (R)-8-bromo-7-chloro-3-cyclobutyl-2-methyl-5-phenyl-2,3,4,5-tetrahydrobenzo[f][1,2,5]thiadiazepine 1,1-dioxide (912.0 mg, 61%). ESI MS m/z=455.0 [M+H]⁺.

Step 6

In a 1 dram vial equipped with a stir bar, (R)-8-bromo-7-chloro-3-cyclobutyl-2-methyl-5-phenyl-2,3,4,5-tetrahydrobenzo[f][1,2,5]thiadiazepine 1,1-dioxide, (R)-8-bromo-7-chloro-3-cyclobutyl-2-methyl-5-phenyl-2,3,4,5-tetrahydrobenzo[f][1,2,5]thiadiazepine 1,1-dioxide (40.0 mg, 0.088 mmol, 1.0 equiv) was combined neat with 2-fluoro-5-(4,4,5,5-tetramethyl-1,3,2-dioxaborolan-2-yl)benzoic acid (30.4 mg, 0.114 mmol, 1.3 equiv, CAS #882679-10-9), cesium carbonate (86.0 mg, 0.26 mmol, 3.0 equiv), and bis(triphenylphosphine)palladium(II) chloride (3.1 mg, 5 mol %) under a nitrogen atmosphere. Next, 1,4-dioxane (0.76 mL) and water (0.11 mL) were added, and the vial was sealed with electrical tape and then heated at 80° C. for 30 min. Upon cooling to room temperature, the reaction mixture was quenched by the addition of formic acid (0.5 mL), concentrated, and purified by RPHPLC. Lyophilization of the isolated product from a mixture of acetonitrile and water afforded (R)-5-(7-chloro-3-cyclobutyl-2-methyl-1,1-dioxido-5-phenyl-2,3,4,5-tetrahydrobenzo[f][1,2,5]thiadiazepin-8-yl)-2-fluorobenzoic acid (26.5 mg, 59%). ESI MS m/z=515.2 [M+H]⁺.

Example 81 was prepared using a procedure analogous to that used above for Ex.80.

| Example | Structure | MS |
|---|---|---|
| 80 | 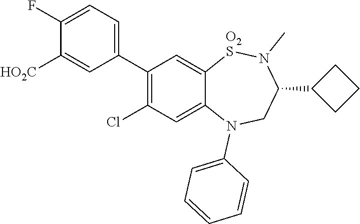 [SMILES: CN1[C@H](C2CCC2)CN(c2ccccc2)c2cc(Cl)c(-c3ccc(F)c(C(=O)O)c3)cc2S1(=O)=O] | [M + H]⁺ 515.2 |
| 81 | 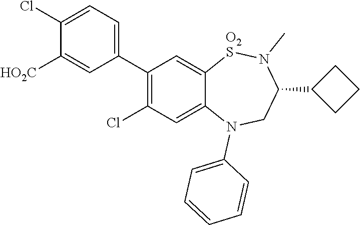 [SMILES: CN1[C@H](C2CCC2)CN(c2ccccc2)c2cc(Cl)c(-c3ccc(Cl)c(C(=O)O)c3)cc2S1(=O)=O] | [M + H]⁺ 531.0 |

### Ex.82: Synthesis of (R)-5-(7-chloro-2-methyl-3-octyl-1,1-dioxido-5-phenyl-2,3,4,5-tetrahydrobenzo[f][1,2,5]thiadiazepin-8-yl)-2-fluorobenzoic acid

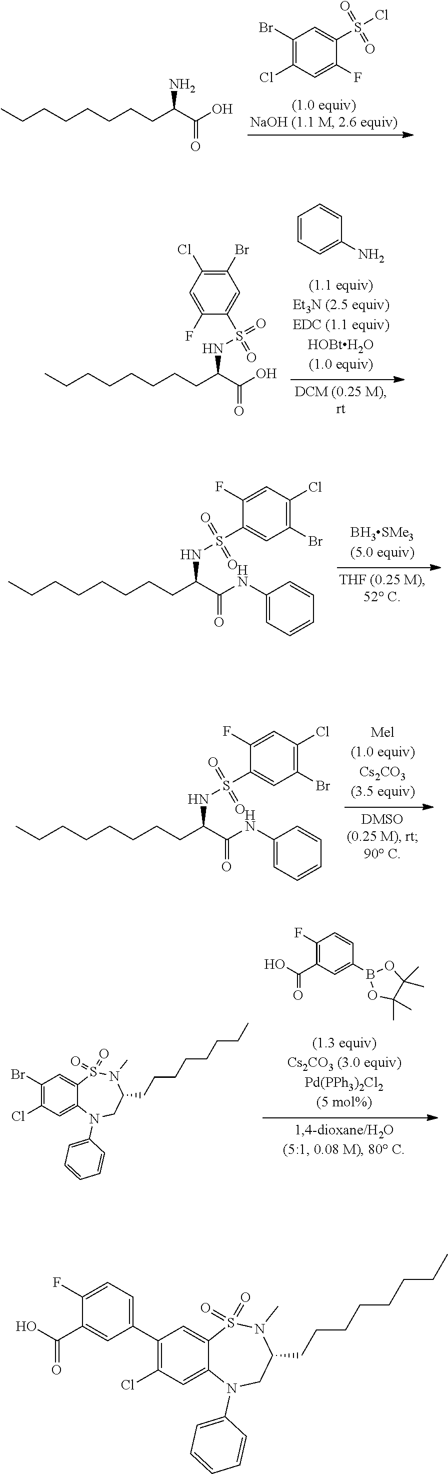

Step 1

In a 40 mL vial equipped with a stir bar, (R)-2-aminodecanoic acid (1.046 g, 5.59 mmol, 1.0 equiv, CAS #84276-16-4) was dissolved in sodium hydroxide (1.1M, 13.2 mL, 2.6 equiv) prior to the addition of 5-bromo-4-chloro-2-fluorobenzenesulfonyl chloride (1.72 g, 5.59 mmol, 1.0 equiv, CAS #1070972-67-6). Upon complete consumption of starting material, as determined by LCMS analysis, the crude reaction mixture was slowly added to 50 mL of 1.2M HCl. The aqueous phase was extracted with ethyl acetate and the combined organic layers were dried over magnesium sulfate. Purification by silica gel column chromatography (ethyl acetate/cyclohexane) afforded (R)-2-((5-bromo-4-chloro-2-fluorophenyl)sulfonamido)decanoic acid (1.068 g). ESI MS m/z=480 [M+Na]⁺.

Step 2

In a 40 mL vial equipped with a stir bar, (R)-2-((5-bromo-4-chloro-2-fluorophenyl)sulfonamido)decanoic acid (1.068 g) was dissolved in dichloromethane (9.3 mL, 0.25M). Aniline (238.0 mg, 0.234 mL, 2.56 mmol, 1.1 equiv) was added, followed by triethylamine (589 mg, 0.811 mL, 5.82 mmol, 2.5 equiv). Next, 1-(3-dimethylaminopropyl)-3-ethylcarbodiimide hydrochloride (0.491 g, 2.56 mmol, 1.1 equiv) and 1H-benzo[d][1,2,3]triazol-1-ol hydrate (0.356 g, 2.327 mmol, 1.0 equiv) were added and the reaction mixture was stirred at room temperature for 25 h. Upon completion, the reaction mixture was concentrated and purified by silica gel column chromatography (ethyl acetate/cyclohexane) to afford (R)-2-((5-bromo-4-chloro-2-fluorophenyl)sulfonamido)-N-phenyldecanamide (595 mg). ESI MS m/z=533.0 [M+H]⁺.

Step 3

In a 40 mL vial equipped with a stir bar, (R)-2-((5-bromo-4-chloro-2-fluorophenyl)sulfonamido)-N-phenyldecanamide (595 mg) was dissolved in tetrahydrofuran (4.46 mL, 0.25M) under a nitrogen atmosphere. Next, borane-dimethylsulfide complex (423 mg, 0.529 mL, 5.57 mmol, 5.0 equiv) was added, and the reaction mixture was heated at 52° C. for 18 h. Upon cooling in an ice bath, the reaction mixture was slowly quenched with 2 mL of water, dried over magnesium sulfate, filtered through a pad of silica gel using ethyl acetate to rinse and concentrated to afford (R)-5-bromo-4-chloro-2-fluoro-N-(1-(phenylamino)decan-2-yl)benzenesulfonamide (537.5 mg) that was used in the next step without purification. ESI MS m/z=519.1 [M+H]⁺.

Step 4

Crude (R)-5-bromo-4-chloro-2-fluoro-N-(1-(phenylamino)decan-2-yl)benzenesulfonamide (537.5 mg) from the previous step was dissolved in dimethyl sulfoxide (4.1 mL, 0.25M). Cesium carbonate (1.18 g, 1.03 mmol, 3.5 equiv) was added, followed by iodomethane (147 mg, 64.6 μL, 1.0 equiv). The reaction mixture was stirred for 15 minutes at room temperature, at which time, LCMS analysis indicated complete methylation to form the tertiary sulfonamide. The mixture was subsequently heated at 90° C. for 1 h to promote the cyclization reaction. Upon cooling to room temperature, the reaction mixture was concentrated using a Biotage® V-10 evaporator and the crude residue was purified by silica gel column chromatography to afford (R)-8-bromo-7-chloro-2-methyl-3-octyl-5-phenyl-2,3,4,5-tetrahydrobenzo[f][1,2,5]thiadiazepine 1,1-dioxide (397.9 mg, 75% yield). ESI MS m/z=515.0 [M+H]⁺.

Step 5

In a 4 mL vial equipped with a stir bar, (R)-8-bromo-7-chloro-2-methyl-3-octyl-5-phenyl-2,3,4,5-tetrahydrobenzo[f][1,2,5]thiadiazepine 1,1-dioxide (40.0 mg, 0.078 mmol, 1.0 equiv), cesium carbonate (76.0 mg, 0.233 mmol, 3.0 equiv), 2-fluoro-5-(4,4,5,5-tetramethyl-1,3,2-dioxaborolan-2-yl)benzoic acid (26.9 mg, 0.101 mmol, 1.3 equiv, CAS #: 867256-77-7), and bis(triphenylphosphine)palladium(II) chloride (2.73 mg, 5 mol %, CAS #: 13965-03-2) were combined neat under a nitrogen atmosphere. Next, 1,4-dioxane (0.78 mL) and water (0.16 mL) were added. The vial was sealed with electrical tape and heated at 80° C. for 4 h. The reaction mixture was quenched with 250 μL of formic acid, passed through a 0.45 micron syringe filter using N,N-dimethylformamide to rinse, and purified by RPHPLC to afford a residue which was lyophilized from an acetonitrile and water mixture to afford, (R)-5-(7-chloro-2-methyl-3-octyl-1,1-dioxido-5-phenyl-2,3,4,5-tetrahydrobenzo[f][1,2,5]thiadiazepin-8-yl)-2-fluorobenzoic acid, as a white solid (23.4 mg, 0.041 mmol, 53% yield). ESI MS m/z=573.2 [M+H]⁺.

Examples 83 and 84 were prepared using a procedure analogous to that used above for Ex.82.

| Example | Structure | MS |
|---|---|---|
| 82 | 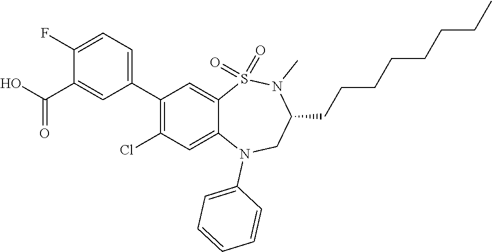 [SMILES: CCCCCCCC[C@@H]1CN(c2ccccc2)c2cc(Cl)c(-c3ccc(F)c(C(=O)O)c3)cc2S(=O)(=O)N1C] | [M + H]⁺ 573.2 |
| 83 | 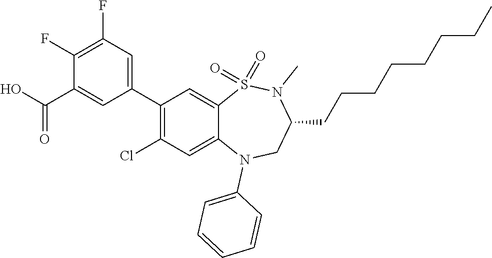 [SMILES: CCCCCCCC[C@@H]1CN(c2ccccc2)c2cc(Cl)c(-c3cc(F)c(F)c(C(=O)O)c3)cc2S(=O)(=O)N1C] | [M + H]⁺ 591.2 |
| 84 | 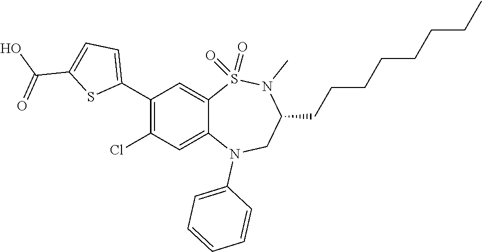 [SMILES: CCCCCCCC[C@@H]1CN(c2ccccc2)c2cc(Cl)c(-c3ccc(C(=O)O)s3)cc2S(=O)(=O)N1C] | [M + H]⁺ 561.1 |

### Ex.85: Synthesis of(R)-5-(3-(tert-butyl)-7-chloro-2-methyl-1,1-dioxido-5-phenyl-2,3,4,5-tetrahydrobenzo[f][1,2,5]thiadiazepin-8-yl)-2-fluorobenzoic acid

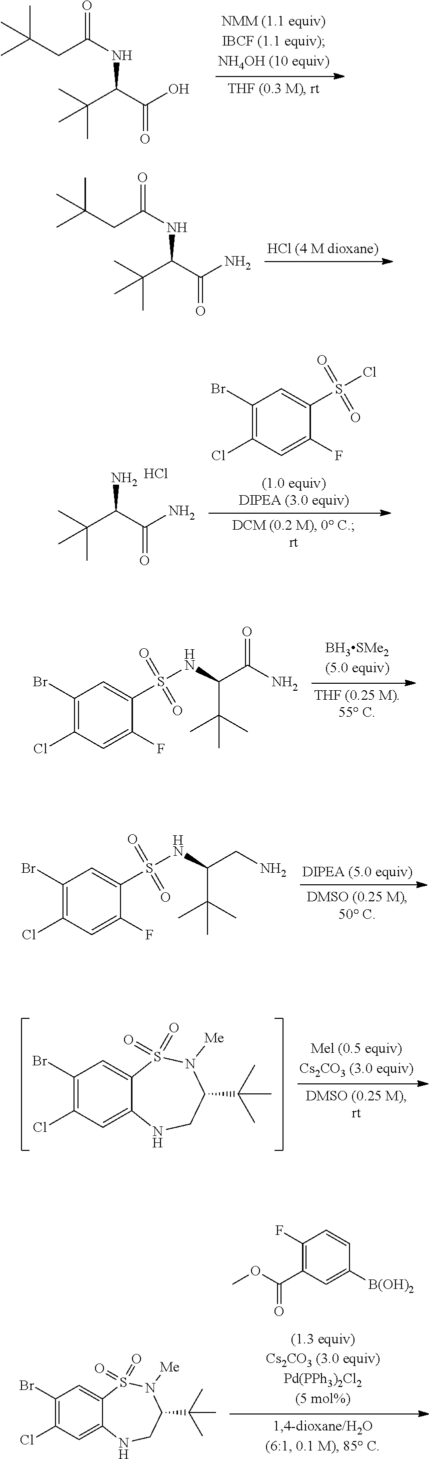

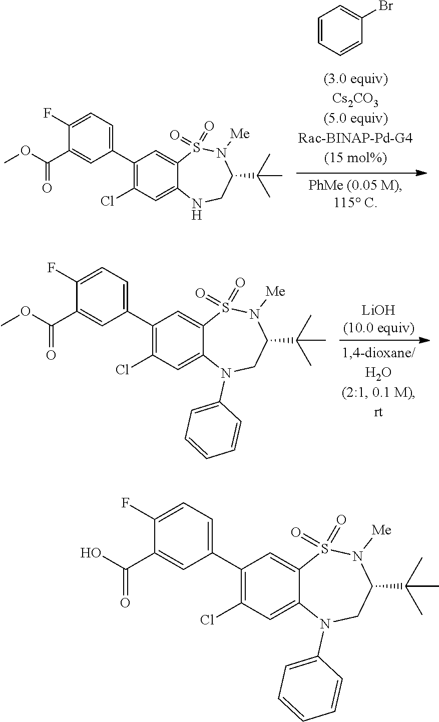

Step 1

In a 100 mL round bottom flask equipped with a stir bar, (R)-2-((tert-butoxycarbonyl)amino)-3,3-dimethylbutanoic acid (2.0 g, 8.65 mmol, 1.0 equiv, CAS #124655-17-0) was dissolved in THE (28.8 mL, 0.3M). To the resulting solution was added N-methylmorpholine (962 mg, 1.05 mL, 9.51 mmol, 1.1 equiv) and isobutyl chloroformate (1.299 g, 1.25 mL, 9.51 mmol, 1.1 equiv). Shortly thereafter, ammonium hydroxide (5.84 mL, 86 mmol, 14.8M, 10 equiv) was added, and the reaction mixture was stirred overnight. Upon completion, the reaction mixture was concentrated in vacuo, and the aqueous phase was extracted three times with ethyl acetate (30 mL per extraction). The combined organic layers were dried over anhydrous sodium sulfate, filtered, and concentrated to afford tert-butyl (R)-(1-amino-3,3-dimethyl-1-oxobutan-2-yl)carbamate (1.99 g theoretical) that was used in the subsequent step without purification. ESI MS m/z=231.0 [M+H]⁺.

Step 2

In a 250 mL round bottom flask equipped with a stir bar, the tert-butyl (R)-(1-amino-3,3-dimethyl-1-oxobutan-2-yl)carbamate (1.99 g theoretical) formed in the previous step was treated with HCl (4M dioxane, 16.2 mL, 7.5 equiv). The reaction mixture was stirred for 4 h, at which time, LCMS analysis indicated full consumption of the starting material. The reaction mixture was concentrated in vacuo to afford (R)-2-amino-3,3-dimethylbutanamide hydrochloride (1.44 g theoretical) which was used in the subsequent step without purification. ESI MS m/z=131.0 [M+H]⁺.

Step 3

In a 250 mL round bottom flask equipped with a stir bar, (R)-2-amino-3,3-dimethylbutanamide hydrochloride (1.44 g theoretical) formed in the previous step was suspended in dichloromethane (43.2 mL, 0.2M) at 0° C. To the resulting suspension was added N,N-diisopropylethylamine (3.35 g, 4.53 mL, 3.0 equiv) which produced a homogenous solution that was subsequently charged with 5-bromo-4-chloro-2-fluorobenzenesulfonyl chloride (2.66 g, 8.65 mmol, 1.0 equiv, CAS #1070972-67-6). The reaction was stirred for 18 h while being allowed to warm to room temperature. The reaction mixture was then diluted with water and 6M HCl producing a white precipitate that was collected via filtration and dried to afford (R)-2-((5-bromo-4-chloro-2-fluorophenyl)sulfonamido)-3,3-dimethylbutanamide (2.30 g, 66% yield). ESI MS m/z=401.0 [M+H]⁺.

Step 4

In a 100 mL round bottom flask equipped with a stir bar and a reflux condenser, (R)-2-((5-bromo-4-chloro-2-fluorophenyl)sulfonamido)-3,3-dimethylbutanamide (2.30 g, 5.73 mmol, 1.0 equiv) was dissolved in tetrahydrofuran (22.9 mL, 0.25M) under a nitrogen atmosphere. Next, borane-dimethyl sulfide complex (2.175 g, 2.72 mL, 5.0 equiv) was added, and the reaction mixture was heated at 55° C. for 18 h. Upon full conversion, as determined by LCMS analysis, the reaction mixture was cooled to room temperature and quenched carefully with methanol over the course of 40 mins. Concentration afforded crude (R)-N-(1-amino-3,3-dimethylbutan-2-yl)-5-bromo-4-chloro-2-fluorobenzenesulfonamide (2.22 g theoretical) which was used in the next step without purification. ESI MS m/z=387.1 [M+H]⁺.

Step 5

The crude (R)-N-(1-amino-3,3-dimethylbutan-2-yl)-5-bromo-4-chloro-2-fluorobenzenesulfonamide (2.22 g theoretical) produced in the previous step was dissolved in dimethylsulfoxide (22.9 mL, 0.25M) in a 100 mL round bottom flask. Next, N,N-diisopropylethylamine (3.70 g, 5.00 mL, 5.0 equiv) was added, and the flask was sealed with a septum then heated at 50° for 27 h at which time, (R)-8-bromo-3(tert-butyl)-7-chloro-2,3,4,5-tetrahydrobenzo[f][1,2,5]thiadiazepine 1,1-dioxide was detected by LCMS and the mixture was cooled to room temperature. ESI MS m/z=367.1 [M+H]⁺.

Step 6

The crude solution of (R)-8-bromo-3-(tert-butyl)-7-chloro-2,3,4,5-tetrahydrobenzo[f][1,2,5]thiadiazepine 1,1-dioxide produced above was charged with cesium carbonate (5.60 g, 17.19 mmol, 3.0 equiv) at room temperature. Next, iodomethane (407 mg, 0.179 mL, 2.87 mmol, 0.5 equiv) was added, and the mixture was stirred at room temperature. for 4 days. The reaction mixture was passed through a pad of celite using dichloromethane to rinse, and the volatiles were removed in vacuo using a Biotage® V-10 evaporator. The resulting orange oil was purified by silica gel column chromatography (ethylacetate/cyclohexane) to afford (R)-8-bromo-3-(tert-butyl)-7-chloro-2-methyl-2,3,4,5-tetrahydrobenzo[f][1,2,5]thiadiazepine 1,1-dioxide (360 mg, 16% yield). ESI MS m/z=381.0 [M+H]⁺.

Step 7

In a 20 mL vial equipped with a stir bar, (R)-8-bromo-3-(tert-butyl)-7-chloro-2-methyl-2,3,4,5-tetrahydrobenzo[f][1,2,5]thiadiazepine 1,1-dioxide (76.0 mg, 0.2 mmol, 1.0 equiv), (4-fluoro-3-(methoxycarbonyl)phenyl)boronic acid (51.0 mg, 0.260 mmol, 1.3 equiv, 874219-35-9), bis(triphenylphosphine)palladium(II) chloride (7.0 mg, 5 mol %), and cesium carbonate (195 mg, 0.60 mmol, 3.0 equiv) were combined neat under a nitrogen atmosphere. Next, 1,4-dioxane (1.7 mL) and water (0.29 mL) were added and the vial was sealed. The reaction was heated at 85° C. for 1 h. The mixture was diluted with water and the aqueous phase was extracted with ethyl acetate. The combined organic layer was filtered through celite and concentrated. The residue was purified by silica gel column chromatography (ethyl acetate/cyclohexane) to afford methyl (R)-5-(3-(tert-butyl)-7-chloro-2-methyl-1,1-dioxido-2,3,4,5-tetrahydrobenzo[f][1,2,5]thiadiazepin-8-yl)-2-fluorobenzoate (80.0 mg, 0.176 mmol, 88% yield). ESI MS m/z=455.1 [M+H]⁺.

Step 8

In a 20 mL vial equipped with a stir bar, (R)-5-(3-(tert-butyl)-7-chloro-2-methyl-1,1-dioxido-2,3,4,5-tetrahydrobenzo[f][1,2,5]thiadiazepin-8-yl)-2-fluorobenzoate (80.0 mg, 0.176 mmol, 1.0 equiv), cesium carbonate (286 mg, 0.879 mmol, 5.0 equiv), and Rac-BINAP-Pd-G4 (26.5 mg, 15 mol %) were combined neat under a nitrogen atmosphere. Next, toluene (3.5 mL, 0.05M) was added followed by bromobenzene (83 mg, 55.6 μL, 3.0 equiv). The vial was sealed and the mixture was heated at 115° C. for 16 h. Upon cooling to room temperature the reaction mixture was passed through a pad of celite using ethyl acetate to rinse and concentrated. The crude residue was purified by silica gel column chromatography (ethyl acetate/hexanes) to afford methyl (R)-5-(3-(tert-butyl)-7-chloro-2-methyl-1,1-dioxido-5-phenyl-2,3,4,5-tetrahydrobenzo[f][1,2,5]thiadiazepin-8-yl)-2-fluorobenzoate (45.0 mg, 4800 yield). ESI MS m/z=531.1 [M+H]⁺.

Step 9

In a 20 mL round bottom flask equipped with a stir bar, (R)-5-(3-(tert-butyl)-7-chloro-2-methyl-1,1-dioxido-5-phenyl-2,3,4,5-tetrahydrobenzo[f][1,2,5]thiadiazepin-8-yl)-2-fluorobenzoate (45.0 mg, 0.085 mmol, 1.0 equiv) was dissolved in a mixture of 1,4-dioxane (0.57 mL) and water (0.28 mL) and lithium hydroxide (20.3 mg, 0.847 mmol, 10.0 equiv). The mixture was stirred at room temperature for 18 h. The crude reaction mixture was purified by RPTIPLC to afford (R)-5-(3-(tert-butyl)-7-chloro-2-methyl-1,1-dioxido-5-phenyl-2,3,4,5-tetrahydrobenzo[f][1,2,5]thiadiazepin-8-yl)-2-fluorobenzoic acid (24.0 mg, 55% yield). ESI MS m/z=517.2 [M+H]⁺.

Examples 86 and 87 were prepared using procedures analogous to that used above for Ex. 85.

| Example | Structure | MS | NMR |
|---|---|---|---|
| 85 | 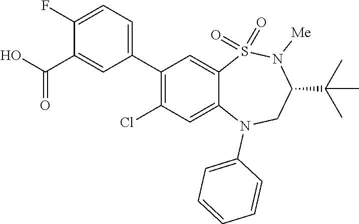 [SMILES: CN1[C@H](C(C)(C)C)CN(c2ccccc2)c2cc(Cl)c(-c3ccc(F)c(C(=O)O)c3)cc2S1(=O)=O] | [M + H]⁺ 517.2 | |
| 86 | 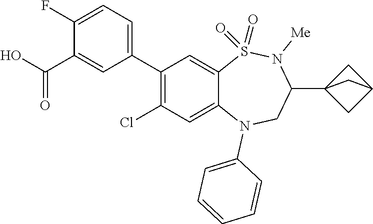 [SMILES: CN1C(C23CC(C2)C3)CN(c2ccccc2)c2cc(Cl)c(-c3ccc(F)c(C(=O)O)c3)cc2S1(=O)=O] | [M + H]⁺ 527.1 | ¹H NMR (400 MHz, CDCl₃) δ 8.14 (dd, J = 6.9, 2.4 Hz, 1H), 7.91 (s, 1H), 7.68 (ddd, J = 8.6, 4.5, 2.5 Hz, 1H), 7.42-7.32 (m, 2H), 7.33-7.23 (m, 1H), 7.14 (s, 1H), 7.12- 7.07 (m, 1H), 7.06-7.00 (m, 2H), 4.12 (dd, J = 15.8, 3.0 Hz, 1H), 3.96 (t, J = 13.5 Hz, 1H), 3.78 (dd, J = 11.0, 3.0 Hz, 1H), 2.91 (s, 3H), 2.59 (s, 1H), 2.20 (s, 1H), 2.03 (s, 1H), 1.91 (s, 6H). |
| 87 | 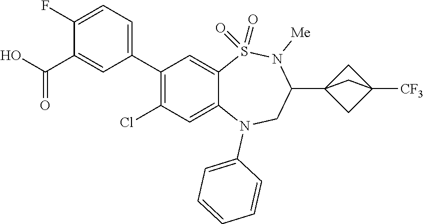 [SMILES: CN1C(C23CC(C(F)(F)F)(C2)C3)CN(c2ccccc2)c2cc(Cl)c(-c3ccc(F)c(C(=O)O)c3)cc2S1(=O)=O] | [M + H]⁺ 595.1 | ¹H NMR (400 MHz, CDCl₃) δ 8.14 (dd, J = 6.8, 2.4 Hz, 1H), 7.89 (s, 1H), 7.68 (ddd, J = 8.5, 4.5, 2.5 Hz, 1H), 7.48-7.40 (m, 2H), 7.32-7.28 (m, 2H), 7.23-7.17 (m, 1H), 7.16-7.11 (m, 2H), 7.04 (s, 1H), 4.50-4.28 (m, 1H), 4.10 (dd, J = 15.8, 3.7 Hz, 1H), 3.74 (dd, J = 11.6, 3.6 Hz, 1H), 3.01 (s, 3H), 2.14-2.02 (m, 6H). |

### Ex.88: Synthesis of 5-((S)-7-chloro-3-((S)-1-methoxyethyl)-2-methyl-1,1-dioxido-5-phenyl-2,3,4,5-tetrahydrobenzo[f][1,2,5]thiadiazepin-8-yl)-2-fluorobenzoic acid

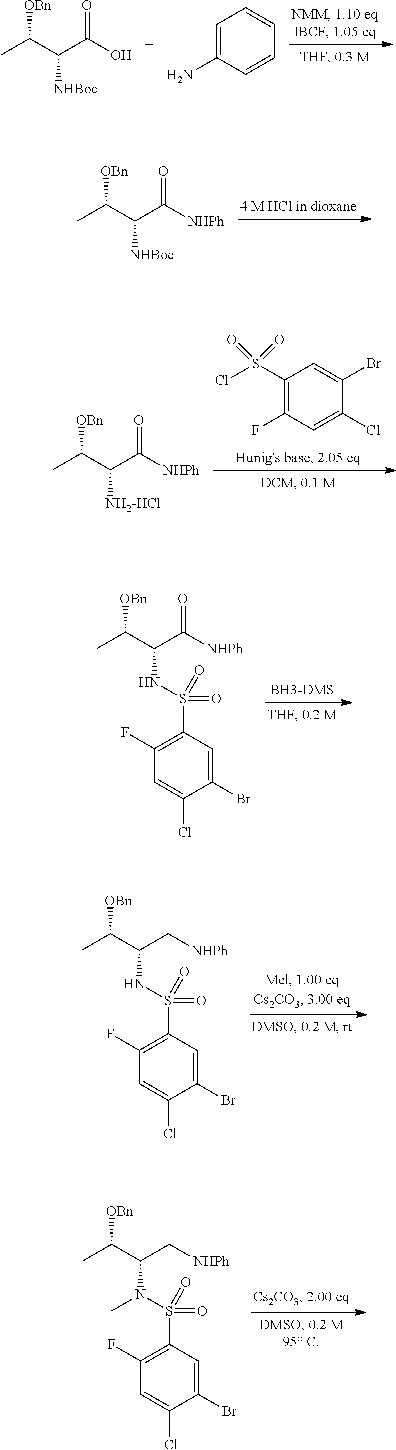

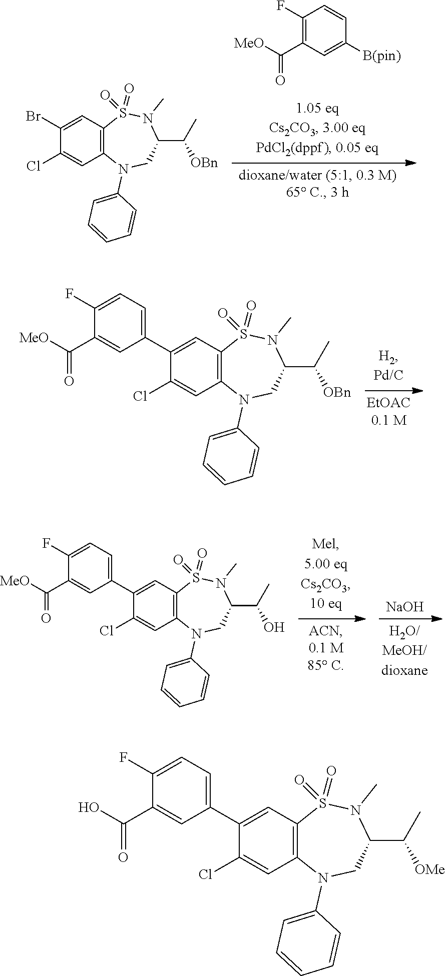

Step 1

To a 500-mL round-bottom flask were added a magnetic stir bar and O-benzyl-N-(tert-butoxycarbonyl)-D-threonine (5 g, 16.16 mmol), followed by THF (54 ml) giving a colorless solution. N-methylmorpholine (1.955 ml, 17.78 mmol) was added, followed by isobutyl chloroformate (2.229 ml, 16.97 mmol), giving a white suspension, which was then treated with aniline (1.623 ml, 17.78 mmol). The resulting white mixture was stirred overnight, at which time LCMS showed complete conversion to product. The reaction mixture was concentrated, taken up in a mixture of DCM/water (1:1), and phase-separated. The combined organics were shaken with an equal volume of water; layers were then separated and the organics concentrated. The resulting crude residue was treated with HCl in dioxane (4.0 M solution, 16.2 ml, 64.6 mmol), giving a yellow solution, which was stirred 2 h, then concentrated under reduced pressure to give a yellow foam, which was further manipulated as follows.

The crude hydrochloride salt (5.18 g, 16.15 mmol) obtained above was charged to a round-bottom flask (500 mL) equipped with a magnetic sir bar. The flask was charged with DCM (90 mL), and the resulting solution treated with Hunig's base (5.79 ml, 33.1 mmol), followed by 5-bromo-4-chloro-2-fluorobenzenesulfonyl chloride (5.03 g, 16.32 mmol). The walls of the flask were rinsed with additional DCM (10 mL), and the deep yellow solution was stirred overnight, then rinsed twice with water, dried over sodium sulfate, filtered and concentrated under reduced pressure into a 500-mL round bottom flask. The resulting brown residue of crude sulfonamide was treated with THE (80 mL) and sonicated, affording a brown solution. The round bottom flask was charged with a magnetic stir bar, flushed with nitrogen, and capped with a rubber septum. Borane-dimethyl sulfide complex (5.37 ml, 56.6 mmol) was added and the reaction mixture heated to 55° C. The reaction mixture was stirred 8 h, at which time LCMS showed complete consumption of starting material. The reaction was cooled to r.t., then quenched by portionwise addition of methanol until gas evolution ceased. The reaction solution was concentrated under reduced pressure to give a brown glass which was used immediately without further purification as described below.

Crude amine (8.76 g, 16.16 mmol) was dissolved in DMSO (81 ml) and charged to a 500-mL round bottom flask containing a magnetic stir bar. Cesium carbonate (15.80 g, 48.5 mmol) was added, the flask sealed with a rubber septum and iodomethane (0.505 ml, 8.08 mmol) was added. The reaction mixture was monitored periodically by LCMS as additional iodomethane (0.515 ml, 8.24 mmol) was added. Once the starting amine had been completely consumed (approximately 8 h after the first addition of iodomethane), additional cesium carbonate (10.5 g, 32.3 mmol) was added, and the reaction mixture was heated at 95° C. Upon full conversion to the cyclized product, as judged by LCMS analysis, the reaction mixture was cooled to r.t. and filtered. The filtrates were diluted with tert-butyl methyl ether (1 L) and the combined organics rinsed with water (5×500 mL). The organic phase was concentrated directly onto silica (60 g) and the residue purified by flash chromatography on silica gel (300 g) eluting with ethyl acetate in cyclohexane to give a white solid (2.44 g, 28%). ¹H NMR (400 MHz, CDCl₃) δ 8.16 (s, 1H), 7.42-7.30 (m, 8H), 7.12 (s, 1H), 7.07 (t, J=7.4 Hz, 1H), 6.96 (d, J=8.0 Hz, 2H), 4.70 (d, J=11.5 Hz, 1H), 4.54 (d, J=11.6 Hz, 1H), 4.09-4.02 (m, 2H), 3.99 (q, J=6.5 Hz, 1H), 3.72-3.61 (m, 1H), 2.97 (s, 3H), 1.27 (d, J=6.4 Hz, 3H). ESI-MS m/z=535.1 [M+H]⁺.

Step 2

To a 20-mL glass vial containing (S)-3-((S)-1-(benzyloxy)ethyl)-8-bromo-7-chloro-2-methyl-5-phenyl-2,3,4,5-tetrahydrobenzo[f][1,2,5]thiadiazepine 1,1-dioxide (1.45 g, 2.71 mmol) and a magnetic stir bar were added cesium carbonate (2.64 g, 8.12 mmol) and methyl 2-fluoro-5-(4,4,5,5-tetramethyl-1,3,2-dioxaborolan-2-yl)benzoate (0.796 g, 2.84 mmol). Dioxane (8 ml) and water (1.5 ml) were added, and the resulting mixture sonicated until solids dissolved. The resulting solution was sparged with nitrogen, then treated with PdCl₂(dppf) (0.099 g, 0.135 mmol). The vial was flushed with nitrogen, capped, placed under positive N₂ pressure and heated to 65° C. After stirring for 3 h, the reaction mixture was concentrated directly onto silica gel and purified by flash chromatography eluting with ethyl acetate in cyclohexane to give the product (1.12 g, 69%) as a white solid. ESI-MS m/z=609.2 [M+H]⁺.

Step 3

A 250-mL round-bottom flask containing methyl 5-((S)-3-((S)-1-(benzyloxy)ethyl)-7-chloro-2-methyl-1,1-dioxido-5-phenyl-2,3,4,5-tetrahydrobenzo[f][1,2,5]thiadiazepin-8-yl)-2-fluorobenzoate (1.02 g, 1.675 mmol) was charged with a magnetic stir bar, 10% (w/w) Pd-C(0.356 g, 0.335 mmol) and ethyl acetate (17 ml). The resulting mixture was sparged with hydrogen, then stirred under a hydrogen atmosphere at balloon pressure. After 48 h, the reaction mixture was filtered through Celite, concentrated under reduced pressure and purified by flash chromatography to afford both recovered starting material and product (0.474 g, 55%) as a white solid. ESI-MS m/z=519.2 [M+H]⁺.

Step 4

To a 20-mL glass vial containing a magnetic stir bar and methyl 5-((S)-7-chloro-3-((S)-1-hydroxyethyl)-2-methyl-1,1-dioxido-5-phenyl-2,3,4,5-tetrahydrobenzo[f][1,2,5]thiadiazepin-8-yl)-2-fluorobenzoate (50 mg, 0.096 mmol) were added cesium carbonate (305 mg, 0.94 mmol), acetonitrile (1 ml) and iodomethane (30 μl, 0.48 mmol). The vial was capped, sealed with tape, and placed in a hotblock pre-heated to 85° C. The reaction mixture was stirred overnight, then concentrated under reduced pressure. Dioxane (0.5 mL), methanol (0.5 mL) and 3 N aqueous NaOH (0.5 mL) were added, and the resulting mixture stirred vigorously for 1.5 h, at which time LCMS indicated complete hydrolysis of the methyl ester. The reaction was quenched with formic acid (0.04 mL), then purified by RP-HPLC to give the product (8 mg, 16%) as a white solid:

| Example | Structure | MS |
|---|---|---|
| 88 | 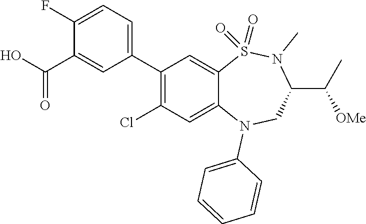 [SMILES: CO[C@@H](C)[C@@H]1CN(c2ccccc2)c2cc(Cl)c(-c3ccc(F)c(C(=O)O)c3)cc2S(=O)(=O)N1C] | [M + H]⁺ 519.3 |

Example 89 was prepare by a procedure identical to that described above for Ex.88, except that in Step 4, ethyl iodide was used in place of iodomethane:

| Example | Structure | MS |
|---|---|---|
| 89 | 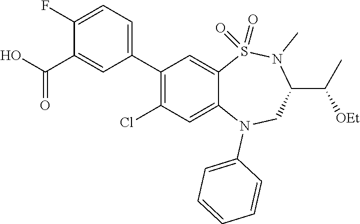 [SMILES: CCO[C@@H](C)[C@@H]1CN(c2ccccc2)c2cc(Cl)c(-c3ccc(F)c(C(=O)O)c3)cc2S(=O)(=O)N1C] | [M + H]⁺ 533.3 |

Likewise, the following compound was prepared by a procedure identical to that described above, except that Step 4 was performed as follows:

To a 20-mL glass vial containing a magnetic stir bar and methyl 5-((S)-7-chloro-3-((S)-1-hydroxyethyl)-2-methyl-1,1-dioxido-5-phenyl-2,3,4,5-tetrahydrobenzo[f][1,2,5]thiadiazepin-8-yl)-2-fluorobenzoate (30 mg, 0.049 mmol) were added dioxane (0.2 mL), methanol (0.2 mL) and 3 N aqueous NaOH (0.2 mL). The resulting mixture was stirred vigorously for 3 h, then quenched with acetic acid (0.04 mL) and purified by RP-HPLC to afford Ex.90 (12 mg, 41%) as a white solid:

| Example | Structure | MS |
|---|---|---|
| 90 | 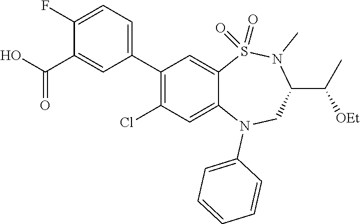 [SMILES: CCO[C@@H](C)[C@@H]1CN(c2ccccc2)c2cc(Cl)c(-c3ccc(F)c(C(=O)O)c3)cc2S(=O)(=O)N1C] | [M − Bn + H]⁺ 505.2 |

Likewise, the following compound was prepared by a procedure identical to that described above, except that Step 4 was performed as follows:

To a 2-dram glass vial containing methyl 5-((S)-7-chloro-3-((S)-1-hydroxyethyl)-2-methyl-1,1-dioxido-5-phenyl-2,3,4,5-tetrahydrobenzo[f][1,2,5]thiadiazepin-8-yl)-2-fluorobenzoate (35 mg, 0.067 mmol) and a magnetic stir bar were added Amberlyst 15-H (50 mg) and DCM (0.7 mL). The mixture was then sparged with isobutylene delivered as a gas through a stainless steel needle to the bottom of the vial; sparging continued for 10 minutes, after which the vial was sealed and stirring continued overnight under an atmosphere of isobutylene. The reaction mixture was then filtered and concentrated under reduced pressure, giving a crude residue which taken up in dioxane (0.25 mL), methanol (0.25 mL) and 3 N aq. NaOH (0.25 mL). The resulting mixture was stirred 3 h, then purified by RP-HIPLC to afford Ex. 91 (8 mg, 21%) as a white solid:

| Example | Structure | MS | NMR |
|---|---|---|---|
| 91 | 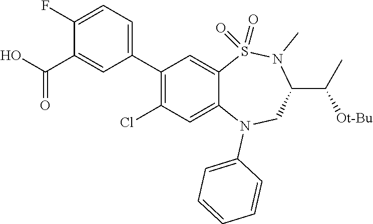 [SMILES: C[C@H](OC(C)(C)C)[C@@H]1CN(c2ccccc2)c2cc(Cl)c(-c3ccc(F)c(C(=O)O)c3)cc2S(=O)(=O)N1C] | [M + H]⁺ 561.2 | ¹H NMR (400 MHz,CDCl₃) δ 8.02 (dd, J = 6.9, 2.4 Hz, 1H), 7.77-7.73 (m, 1H), 7.59-7.53 (m, 1H), 7.34 (d, J = 8.5 Hz, 2H), 7.17-7.10 (m, 2H), 7.02 (d, J = 8.5 Hz, 2H), 6.88 (s, 1H), 4.59-4.45 (m, 1H), 4.02 (dd, J = 16.0, 4.1 Hz, 1H), 3.95 (t, J = 6.5 Hz, 1H), 3.30 (dd, J = 9.8, 5.0 Hz, 1H), 3.16 (s, 1H), 3.02 (s, 3H), 1.29 (s, 9H), 1.17 (d, J = 6.3 Hz, 3H). |

Likewise, the following compound was prepared by a procedure identical to that described above, except that Step 4 was performed as follows:

To a 20-mL glass vial containing a magnetic stir bar and methyl 5-((S)-7-chloro-3-((S)-1-hydroxyethyl)-2-methyl-1,1-dioxido-5-phenyl-2,3,4,5-tetrahydrobenzo[f][1,2,5]thiadiazepin-8-yl)-2-fluorobenzoate (100 mg, 0.193 mmol) were added di-tert-butyl azodicarboxylate (91 mg, 0.393 mmol), triphenylphosphine (bead-supported, 1.6 mmol/gram, 246 mg, 0.393 mmol), THE (2 mL) and acetic acid (24 mg, 0.393 mmol). The resulting mixture was stirred 24 h, then filtered and concentrated under reduced pressure. The residue was dissolved in dioxane (1 mL), methanol (1 mL), and 3 N aq. NaOH (1 mL), then stirred one week at room temperature. The reaction mixture was concentrated under reduced pressure to give the crude sodium carboxylate which was taken up in minimal DCM and absorbed to silica. Impurities were removed by rinsing with DCM, followed by EtOAc, followed by additional DCM; the desired material was then eluted with methanol. The methanolic filtrates were concentrated, and the resulting crude material was charged to a 2-dram vial containing a magnetic stir bar. DMSO (0.5 mL), cesium carbonate (194 mg, 0.594 mmol), ethyl iodide (93 mg, 0.594 mmol) were added, and the resulting mixture stirred 24 h at 45° C.; sodium hydride (12 mg, 60% w/w dispersion in mineral oil, 0.297 mmol) was then added and the resulting mixture stirred overnight. The reaction mixture was quenched with acetic acid (0.02 mL, 0.297 mmol), filtered and purified by RP-HPLC to afford Ex 92 (5 mg, 5%) as a white solid:

| Example | Structure | MS | NMR |
|---|---|---|---|
| 92 | 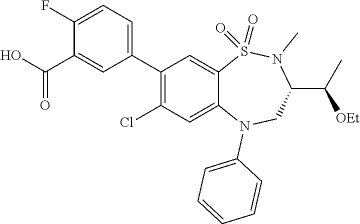 [SMILES: CCO[C@H](C)[C@@H]1CN(c2ccccc2)c2cc(Cl)c(-c3ccc(F)c(C(=O)O)c3)cc2S(=O)(=O)N1C] | [M + H]⁺ 533.2 | ¹H NMR (400 MHz, CDCl₃) δ 8.11 (dd, J = 6.9, 2.5 Hz, 1H), 7.86 (s, 1H), 7.65 (ddd, J = 8.6, 4.5, 2.4 Hz, 1H), 7.37 (dd, J = 8.3, 7.2 Hz, 2H), 7.28-7.21 (m, 1H), 7.17-7.06 (m, 4H), 4.37 (s, 1H), 3.85-3.74 (m, 1H), 3.61 (t, J = 7.9 Hz, 1H), 3.48-3.09 (m, 3H), 2.96 (s, 3H), 1.31 (d, J = 6.1 Hz, 3H), 1.15 (t, J = 6.8 Hz, 2H). |

### Ex.93: Synthesis of 5-((S)-7-chloro-3-((S)-1-methoxyethyl)-2-methyl-1,1-dioxido-5-phenyl-2,3,4,5-tetrahydrobenzo[f][1,2,5]thiadiazepin-8-yl)-2-fluorobenzoic acid

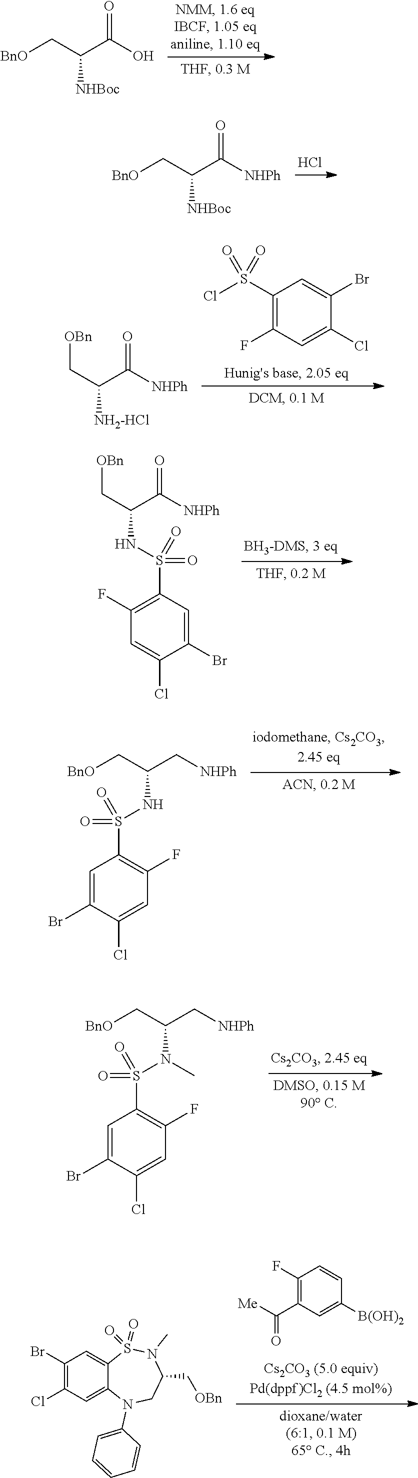

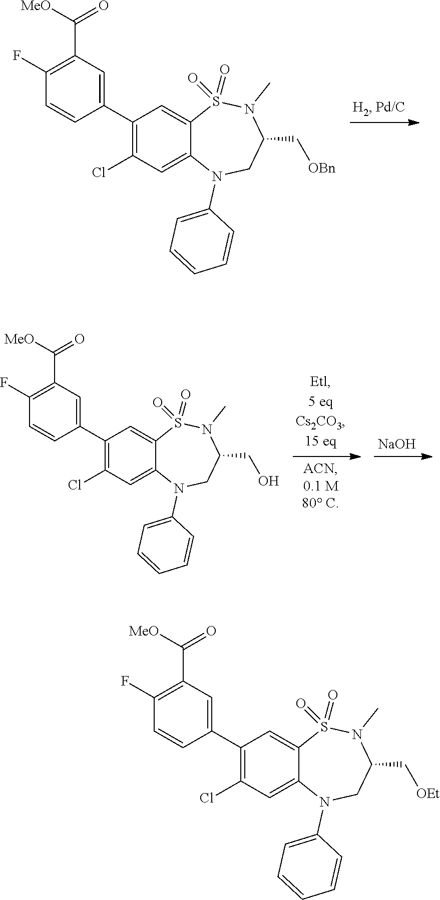 [SMILES: COC(=O)c1cc(-c2cc3c(cc2Cl)N(c2ccccc2)C[C@@H](COCc2ccccc2)N(C)S3(=O)=O)ccc1F ; COC(=O)c1cc(-c2cc3c(cc2Cl)N(c2ccccc2)C[C@@H](CO)N(C)S3(=O)=O)ccc1F ; "NaOH" ; CCOC[C@@H]1CN(c2ccccc2)c2cc(Cl)c(-c3ccc(F)c(C(=O)OC)c3)cc2S(=O)(=O)N1C]

Step 1

To a 500-mL round bottom flask containing a magnetic stir bar and O-benzyl-N-(tert-butoxycarbonyl)-D-serine (10 g, 33.9 mmol) was added THF (113 ml), giving a colorless solution. N-methylmorpholine (4.09 ml, 37.2 mmol) was added, followed by isobutyl chloroformate (4.67 ml, 35.6 mmol), giving a white suspension. The reaction vessel was placed in an ice bath and aniline (3.40 ml, 37.2 mmol) was added. After 30 min, the ice bath was removed and the reaction mixture stirred overnight, at which time LCMS confirmed complete consumption of starting material. The reaction mixture was concentrated under reduced pressure and the resulting residue partitioned between DCM (100 mL) and water (100 mL) and phase-separated. The aqueous phase was partially extracted once more with DCM, then the combined organics were rinsed with water, phase-separated and concentrated under reduced pressure to give a viscous yellow oil which was charged to a round bottom flask equipped with a magnetic stir bar, dissolved in a solution of HCl in dioxane (4.0 M, 42.4 mmol, 170 mmol) and stirred vigorously at room temperature for 30 min. The resulting yellow solution was concentrated under reduced pressure, affording the crude amine hydrochloride as a brownish-yellow gum which was dissolved in DCM (150 mL), then treated with Hunig's base (12 mL, 69.5 mmol) and 5-bromo-4-chloro-2-fluorobenzenesulfonyl chloride (10.65 g, 34.6 mmol). The resulting deep yellow solution was stirred overnight, then rinsed with water (3×100 mL), dried with sodium sulfate, filtered and concentrated under reduced pressure to give the crude sulfonamide, which was used immediately without further purification as follows:

To a 500-mL round bottom flask containing the material obtained above was added THE (170 mL), affording a yellow solution. The flask was blown out with nitrogen, capped with a rubber septum and charged with BH₃-DMS (9.66 ml, 102 mmol). After 3 h, the reaction flask was heated to 40° C.; after stirring overnight, the reaction mixture was cooled to r.t. and treated with methanol portionwise until gas evolution ceased. The resulting solution was concentrated under reduced pressure to give (S)-N-(1-(benzyloxy)-3-(phenylamino)propan-2-yl)-5-bromo-4-chloro-2-fluorobenzenesulfonamide as an off-white, waxy solid (18 g, 100%). ESI-MS m/z=527.0 [M+H]⁺.

Step 2

To a 500-mL round bottom flask containing (S)-N-(1-(benzyloxy)-3-(phenylamino)propan-2-yl)-5-bromo-4-chloro-2-fluorobenzenesulfonamide (17.52 g, 33.2 mmol) were added acetonitrile (200 mL) and cesium carbonate (27.0 g, 83 mmol). The flask was capped with a rubber septum, iodomethane (2.076 mL, 33.2 mmol) was added, and the resulting mixture stirred at r.t. for 2 h. Volatiles were then removed under reduced pressure, and the flask charged with additional cesium carbonate (27.0 g, 83 mmol) and DMSO (166 ml). The resulting mixture was first sonicated to render it homogenous, then heated to 90° C. and stirred 4 h, at which time LCMS showed complete conversion to the desired product. The reaction was allowed to cool, then diluted with diethyl ether (300 mL), filtered to remove most of the cesium carbonate, and further diluted with tert-butyl methyl ether (1.2 L). The organics were washed with water (4×500 mL) and brine (1×500 mL), then concentrated under reduced pressure onto silica gel (60 g). Purification by flash chromatography on silica gel eluting with ethyl acetate in cyclohexane gave (S)-3-((benzyloxy)methyl)-8-bromo-7-chloro-2-methyl-5-phenyl-2,3,4,5-tetrahydrobenzo[f][1,2,5]thiadiazepine 1,1-dioxide (11.23 g, 65%) as an off-white foam. ESI-MS m z=521.0 [M+H]⁺. ¹H NMR (400 MHz, CDCl₃) δ 8.13 (s, 1H), 7.40-7.21 (m, 7H), 7.11-7.05 (m, 2H), 7.05-7.00 (m, 2H), 4.47 (s, 2H), 4.11 (s, 2H), 3.84-3.65 (m, 3H), 2.90 (s, 3H).

Step 3

A 500-mL round bottom flask was charged with (S)-3-((benzyloxy)methyl)-8-bromo-7-chloro-2-methyl-5-phenyl-2,3,4,5-tetrahydrobenzo[f][1,2,5]thiadiazepine 1,1-dioxide (3.83 g, 7.34 mmol), cesium carbonate (11.96 g, 36.7 mmol), (4-fluoro-3-(methoxycarbonyl)phenyl)boronic acid (1.525 g, 7.71 mmol) and a magnetic stir bar. Dioxane (62 ml) and water (10 ml) were added, and the resulting burnt-orange colored solution was sparged with nitrogen for 10 min. PdCl₂(dppf) (0.269 g, 0.367 mmol) was added. The flask was sealed with a rubber septum and heated to 65° C. After 3.5 h, LCMS analysis indicated full conversion. Upon cooling to room temperature, the reaction mixture was concentrated onto silica gel (60 g) and purified by flash chromatography to afford methyl (S)-5-(3-((benzyloxy)methyl)-7-chloro-2-methyl-1,1-dioxido-5-phenyl-2,3,4,5-tetrahydrobenzo[f][1,2,5]thiadiazepin-8-yl)-2-fluorobenzoate (3.49 g, 80%) as a white solid. ESI-MS m/z=595.2 [M+H]⁺.

Step 4

A 250-mL round bottom flask was charged with methyl (S)-5-(3-((benzyloxy)methyl)-7-chloro-2-methyl-1,1-dioxido-5-phenyl-2,3,4,5-tetrahydrobenzo[f][1,2,5]thiadiazepin-8-yl)-2-fluorobenzoate (2.85 g, 4.79 mmol), a magnetic stir bar, and 10% Pd-C(1.019 g, 0.958 mmol), followed by ethyl acetate (50 mL). The resulting mixture was sparged with hydrogen for 5 min, then stirred under a hydrogen atmosphere at balloon pressure an additional 8 h. The reaction mixture was then filtered through Celite and concentrated under reduced pressure to afford methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (1.93 g, 80%) as a white solid. ESI-MS m/z=505.2 [M+H]⁺. ¹H NMR (400 MHz, CDCl₃) δ 8.01 (dd, J=6.8, 2.4 Hz, 1H), 7.90-7.83 (m, 1H), 7.60 (ddd, J=8.4, 4.6, 2.4 Hz, 1H), 7.39 (t, J=7.8 Hz, 2H), 7.25-7.11 (m, 4H), 7.07-7.01 (m, 1H), 4.44-4.34 (m, 1H), 4.19-4.08 (m, 1H), 3.96 (s, 3H), 3.94-3.88 (m, 1H), 3.84-3.77 (m, 1H), 3.70 (s, 1H), 3.01 (s, 3H).

Step 5

To a 2-dram glass vial equipped with a magnetic stir bar and containing methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (16 mg, 0.032 mmol) were added cesium carbonate (100 mg, 0.32 mmol), acetonitrile (0.3 mL) and ethyl iodide (25 mg, 0.158 mmol). The vial was capped, sealed with electrical tape, and heated to 80° C. After stirring overnight, the reaction mixture was diluted with DCM (1 mL), filtered, and concentrated; the residues thus obtained were then taken up in dioxane (0.4 mL), methanol (0.2 mL) and 3 N aqueous NaOH (0.2 mL). The resulting mixture was stirred at room temperature 1.5 hours, then quenched with acetic acid and purified by RP-HPLC to afford (S)-5-(7-chloro-3-(ethoxymethyl)-2-methyl-1,1-dioxido-dioxido-5-phenyl-2,3,4,5-tetrahydrobenzo[f][1,2,5]thiadiazepin-8-yl)-2-fluorobenzoic acid, Ex. 93, (2 mg, 10%) as a white solid.

| Example | Structure | MS |
|---|---|---|
| 93 | 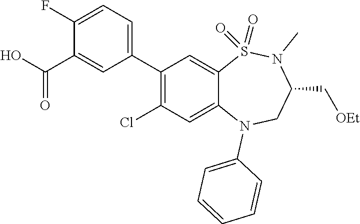 [SMILES: CCOC[C@@H]1CN(c2ccccc2)c2cc(Cl)c(-c3ccc(F)c(C(=O)O)c3)cc2S(=O)(=O)N1C] | [M + H]⁺ 519.2 |

Likewise, the following compounds were prepared by a procedure identical to that described above, except that in Step 5, an appropriate alkyl halide was used in place of ethyl iodide:

| Example | Structure | MS |
|---|---|---|
| 94 | 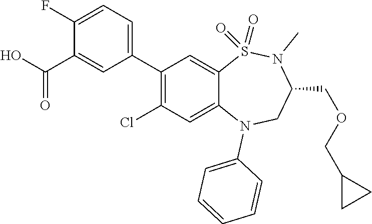 | [M + H]⁺ 559.4 |
| 95 | 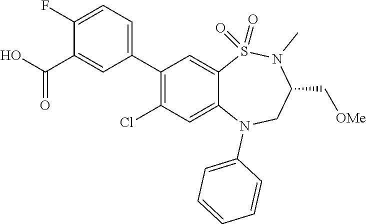 [SMILES: COC[C@@H]1CN(c2ccccc2)c2cc(Cl)c(-c3ccc(F)c(C(=O)O)c3)cc2S(=O)(=O)N1C] | [M + H]⁺ 505.2 |

Likewise, the following compound was prepared by a procedure identical to that described above, except that Step 5 was performed as follows:

To an oven-dried 2-dram glass vial equipped with a dry magnetic stir bar were added methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (30 mg, 0.059 mmol), potassium hydride (30% w/w dispersion in mineral oil, 40 mg, 0.297 mmol), 2-iodopropane (101 mg, 0.594 mmol), and anhydrous DMSO (0.5 mL). The vial was sealed, heated to 95° C., and the reaction mixture stirred vigorously for 2 weeks. The dark brown reaction mixture was then allowed to cool to r.t., treated with 3 N aqueous NaOH (0.25 mL), and allowed to stir overnight, at which time the reaction mixture was neutralized with acetic acid, filtered and purified by RP-HPLC to provide (S)-5-(7-chloro-3-(isopropoxymethyl)-2-methyl-1,1-dioxido-5-phenyl-2,3,4,5-tetrahydrobenzo[f][1,2,5]thiadiazepin-8-yl)-2-fluorobenzoic acid, Ex.96, (3 mg, 9.5%) as a white solid:

| Example | Structure | MS |
|---|---|---|
| 96 | 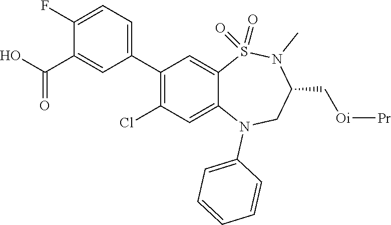 | [M + H]⁺ 533.2 |

Likewise, the following compound was prepared by a procedure identical to that described above, except that Step 5 was performed as follows:

To a 2-dram vial containing a magnetic stir bar and methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (16 mg, 0.032 mmol) were added DCM (0.3 mL), Hunig's base (7.19 μl, 0.041 mmol), and methoxymethyl chloride (3.61 μl, 0.038 mmol). The resulting colorless solution was stirred overnight, concentrated, re-constituted in dioxane (0.2 mL), MeOH (0.2 mL) and 3 N aq. NaOH (0.2 mL), and stirred until LCMS indicated complete consumption of starting material, then purified by RP-HPLC to afford (S)-5-(7-chloro-3-((methoxymethoxy)methyl)-2-methyl-1,1-dioxido-5-phenyl-2,3,4,5-tetrahydrobenzo[f][1,2,5]thiadiazepin-8-yl)-2-fluorobenzoic acid, Ex.97, (11 mg, 62%) as a white solid:

| Example | Structure | MS |
|---|---|---|
| 97 | 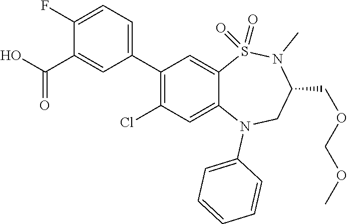 [SMILES: COCOC[C@@H]1CN(c2ccccc2)c2cc(Cl)c(-c3ccc(F)c(C(=O)O)c3)cc2S(=O)(=O)N1C] | [M − OMe]⁺ 503.0 |

Likewise, the following compound was prepared by a procedure identical to that described above, except that Step 5 was performed as follows:

To a 2-dram glass vial containing methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (15 mg, 0.03 mmol) and a magnetic stir bar were added dioxane (0.2 mL), methanol (0.2 mL) and 3 N aqueous NaOH (0.2 mL). The resulting mixture was stirred overnight, then quenched with acetic acid and purified by RP-HPLC to afford (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoic acid, Ex.98, (8.4 mg, 58%) as a white solid:

| Example | Structure | MS |
|---|---|---|
| 98 | 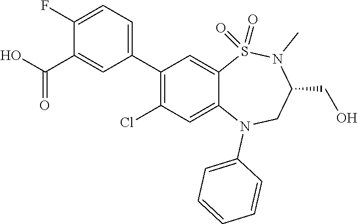 [SMILES: CN1[C@H](CO)CN(c2ccccc2)c2cc(Cl)c(-c3ccc(F)c(C(=O)O)c3)cc2S1(=O)=O] | [M + H]⁺ 491.3 |

Likewise, the following compound was prepared by a procedure identical to that described above, except that Step 5 was performed as follows:

To a 2-dram glass vial containing methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (35 mg, 0.069 mmol) and a magnetic stir bar were added THE (0.7 mL), diphenyl phosphoryl azide (37 mg, 0.15 mmol), and triphenylphosphine (43 mg, 0.17 mmol). The resulting mixture was stirred 2 h, at which time the reaction mixture was concentrated under reduced pressure and divided into two aliquots. To a 2-dram vial containing the first aliquot were charged a magnetic stir bar, dioxane (0.2 mL), methanol (0.2 mL) and 3 N aqueous NaOH (0.2 mL); the resulting mixture was stirred 3 h, then purified by RP-HPLC to give (S)-5-(3-(azidomethyl)-7-chloro-2-methyl-1,1-dioxido-5-phenyl-2,3,4,5-tetrahydrobenzo[f][1,2,5]thiadiazepin-8-yl)-2-fluorobenzoic acid, Ex.99 (5.5 mg, 31%) as a white solid:

| Example | Structure | MS |
|---|---|---|
| 99 | 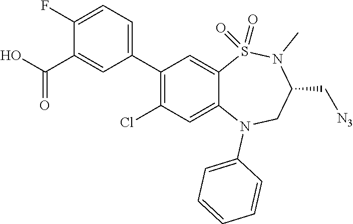 [SMILES: CN1[C@H](CN=[N+]=[N-])CN(c2ccccc2)c2cc(Cl)c(-c3ccc(F)c(C(=O)O)c3)cc2S1(=O)=O] | [M + H]⁺ 516.1 |

Likewise, the following compound was prepared by a procedure identical to that described above, except that Step 5 was performed as follows: To a 2-dram glass vial containing methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (15 mg, 0.03 mmol) and a magnetic stir bar were added phenol (5.3 mg, 0.056 mmol), triphenylphosphine (8 mg, 0.030 mmol), diisopropyl azodicarboxylate (6.6 mg, 0.033 mmol), and THE (0.3 mL). The resulting mixture was stirred overnight, then treated with 3 N aqueous NaOH (0.3 mL) and stirred until LCMS indicated complete conversion to product. The reaction mixture was then neutralized with acetic acid, concentrated under reduced pressure, and purified by RP-HPLC to afford (S)-5-(7-chloro-2-methyl-1,1-dioxido-3-(phenoxymethyl)-5-phenyl-2,3,4,5-tetrahydrobenzo[f][1,2,5]thiadiazepin-8-yl)-2-fluorobenzoic acid, Ex.100 (6 mg, 36%) as a white solid:

| Example | Structure | MS |
|---|---|---|
| 100 | 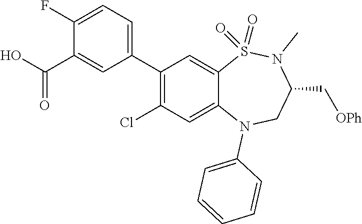 [SMILES: CN1[C@H](COc2ccccc2)CN(c2ccccc2)c2cc(Cl)c(-c3ccc(F)c(C(=O)O)c3)cc2S1(=O)=O] | [M + H]⁺ 567.2 |

Likewise, the following compound was prepared by a procedure identical to that described above, except that Step 5 was performed as follows:

In a 2-dram glass vial were combined methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (19 mg, 0.038 mmol), Amberlyst 15-H (100 mg), a magnetic stir bar and DCM (0.4 mL); the resulting mixture was sparged with isobutylene for 10 minutes, then allowed to stir under an isobutylene atmosphere 9 h. The reaction mixture was then filtered, concentrated under reduced pressure, and re-constituted in dioxane (0.3 mL), methanol (0.3 mL) and 3 N aqueous NaOH (0.3 mL). The resulting mixture was stirred 3 h, then purified by RP-HPLC, affording (S)-5-(3-(tert-butoxymethyl)-7-chloro-2-methyl-1,1-dioxido-5-phenyl-2,3,4,5-tetrahydrobenzo[f][1,2,5]thiadiazepin-8-yl)-2-fluorobenzoic acid, Ex.101, (6 mg, 29%) as a white solid:

| Example | Structure | MS |
|---|---|---|
| 101 | 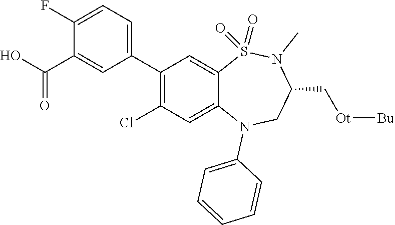 | [M − C₄H₈ + H]⁺ 491.1 |

Likewise, the following compound was prepared by a procedure identical to that described above, except that Steps 4-5 were performed as follows:

To a 2-dram glass vial equipped with a magnetic stir bar and containing methyl (S)-5-(3-((benzyloxy)methyl)-7-chloro-2-methyl-1,1-dioxido-5-phenyl-2,3,4,5-tetrahydrobenzo[f][1,2,5]thiadiazepin-8-yl)-2-fluorobenzoate (23 mg, 0.039 mmol) were added dioxane (0.15 mL), methanol (0.15 mL) and 3 N aqueous NaOH (0.15 mL). The resulting mixture was stirred 3 h at r.t., then quenched with acetic acid (0.02 mL) and purified by RP-HPLC to afford 5-((S)-3-((S)-1-(benzyloxy)ethyl)-7-chloro-2-methyl-1,1-dioxido-5-phenyl-2,3,4,5-_tetrahydrobenzo[f][1,2,5]thiadiazepin-8-yl)-2-fluorobenzoic acid, Ex.102 (18 mg, 80%) as a white solid:

| Example | Structure | MS |
|---|---|---|
| 102 | 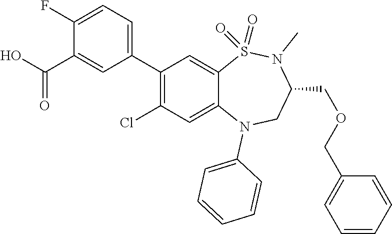 [SMILES: CN1[C@H](COCc2ccccc2)CN(c2ccccc2)c2cc(Cl)c(-c3ccc(F)c(C(=O)O)c3)cc2S1(=O)=O] | [M + H]⁺ 581.2 |

### Ex.103: Synthesis of 5-((3S)-7-chloro-3-(3,6-dihydro-2H-pyran-2-yl)-2-methyl-1,1-dioxido-5-phenyl-2,3,4,5-tetrahydrobenzo[f][1,2,5]thiadiazepin-8-yl)-2-fluorobenzoic acid

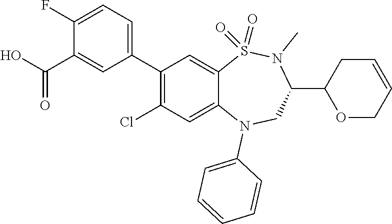 [SMILES: CN1[C@H](C2CC=CCO2)CN(c2ccccc2)c2cc(Cl)c(-c3ccc(F)c(C(=O)O)c3)cc2S1(=O)=O]

Step 1

To a 500-mL round bottom flask containing methyl (S)-5-(7-chloro-3-(hydroxymethyl)-2-methyl-1,1-dioxido-dioxido-5-phenyl-2,3,4,5-tetrahydrobenzo[f][1,2,5]thiadiazepin-8-yl)-2-fluorobenzoate (1.93 g, 3.82 mmol) and a magnetic stir bar was added DCM (30 mL), giving a colorless stirred solution. Dess-Martin periodinane (3.24 g, 7.64 mmol) was added, the mixture subsequently evolving to a red-brown, then brown opaque appearance. The reaction mixture was stirred 45 minutes, at which time LCMS showed complete conversion to product. The reaction was quenched with ethanol (0.402 ml, 6.88 mmol), then concentrated directly onto silica gel (30 g). Flash chromatography gave methyl (S)-5-(7-chloro-3-formyl-2-methyl-1,1-dioxido-5-phenyl-2,3,4,5-tetrahydrobenzo[f][1,2,5]thiadiazepin-8-yl)-2-fluorobenzoate as a white powder; a portion of this material was carried on as follows:

Methyl (S)-5-(7-chloro-3-formyl-2-methyl-1,1-dioxido-5-phenyl-2,3,4,5-tetrahydrobenzo[f][1,2,5]thiadiazepin-8-yl)-2-fluorobenzoate (10 mg, 0.020 mmol) was charged to a 2-dram glass vial equipped with a magnetic stir bar. Indium dust (6.85 mg, 0.060 mmol), allyl bromide (7.22 mg, 0.060 mmol), THE (0.32 ml) and water (0.08 ml) were added, and the colorless mixture stirred 15 h, at which time an additional drop of allyl bromide was added. The resulting grey suspension was sonicated, then stirred a further 5 h, at which time the reaction was quenched with 1 N aqueous HCl (0.1 mL, 0.1 mmol) and extracted with ethyl acetate (3×0.25 mL). The combined organics were transferred to a 3-dram glass vial and concentrated, affording a colorless residue which was carried on immediately as described below:

To the 2-dram glass vial containing the crude allyl alcohol obtained as described above were added a magnetic stir bar, cesium carbonate (0.033 g, 0.100 mmol), and acetonitrile (0.2 ml), followed by allyl bromide (0.03 mL, 0.3 mmol). The resulting opaque white mixture was stirred at room temperature 96 h, at which time the reaction mixture was concentrated and partitioned between water and ethyl acetate. The layers were separated and the aqueous phase extracted three times with ethyl acetate; the combined organics were then concentrated to afford the crude diene, methyl 5-((3S)-3-(1-(allyloxy)but-3-en-1-yl)-7-chloro-2-methyl-1,1-dioxido-5-phenyl-2,3,4,5-tetrahydrobenzo[f][1,2,5]thiadiazepin-8-yl)-2-fluorobenzoate, as a colorless solid (10 mg, 85%). ESI-MS m/z=585.3 [M+H]⁺.

Step 2

To a 2-dram glass vial containing methyl 5-((3S)-3-(1-(allyloxy)but-3-en-1-yl)-7-chloro-2-methyl-1,1-dioxido-dioxido-5-phenyl-2,3,4,5-tetrahydrobenzo[f][1,2,5]thiadiazepin-8-yl)-2-fluorobenzoate (10 mg, 0.017 mmol) and a magnetic stir bar were added Grubbs Catalyst M204 (3.63 mg, 4.27 μmol) and DCM (0.9 mL), giving a brown solution which was sparged with nitrogen. The vial was then capped, sealed and placed in a hotblock preheated to 45° C. The reaction solution was stirred overnight, then additional catalyst (3.63 mg, 3.53) was added and the reaction was stirred an additional 24 h. The reaction mixture was concentrated, and the resulting residue taken up in dioxane (0.25 mL), methanol (0.25 mL), and 3 N aqueous NaOH (0.25 mL), affording a brown mixture which was stirred an additional 1.5 h. The reaction mixture was quenched with acetic acid and purified by RP-HPLC to afford 5-((3S)-7-chloro-3-(3,6-dihydro-2H-pyran-2-yl)-2-methyl-1,1-dioxido-5-phenyl-2,3,4,5-tetrahydrobenzo[f][1,2,5]thiadiazepin-8-yl)-2-fluorobenzoic acid (4 mg, 43%) as a colorless, apparently diastereopure solid; the relative configuration of the two stereocenters was not determined:

| Example | Structure | MS | NMR |
|---|---|---|---|
| 103 | 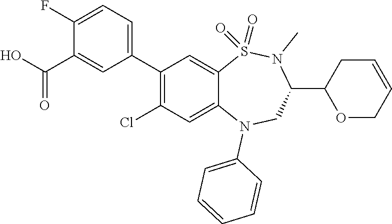 [SMILES: CN1[C@H](C2CC=CCO2)CN(c2ccccc2)c2cc(Cl)c(-c3ccc(F)c(C(=O)O)c3)cc2S1(=O)=O] | [M + H]⁺ 543.3 | ¹H NMR (400 MHz, CDCl₃) δ 8.11 (dd, J = 6.9, 2.4 Hz, 1H), 7.87 (s, 1H), 7.66 (ddd, J = 8.6, 4.5, 2.5 Hz, 1H), 7.41- 7.32 (m, 2H), 7.29-7.22 (m, 1H), 7.18-7.03 (m, 4H), 5.87- 5.78 (m, 1H), 5.75-5.65 (m, 1H), 4.38 (br s, 2H), 4.11 (br s, 2H), 3.89 (td, J = 9.6, 3.2 Hz, 1H), 3.41 (s, 1H), 2.98 (s, 3H), 2.43 (d, J = 17.6 Hz, 1H), 2.09 (t, J = 14.5 Hz, 1H). |

### Ex.104: Synthesis of 5-((3S)-7-chloro-2-methyl-1,1-dioxido-5-phenyl-3-(tetrahydro-2H-pyran-2-yl)-2,3,4,5-tetrahydrobenzo[f][1,2,5]thiadiazepin-8-yl)-2-fluorobenzoic acid

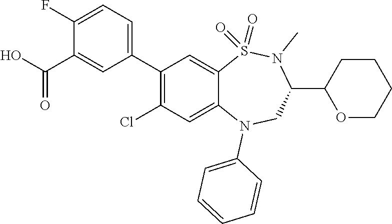 [SMILES: CN1[C@H](C2CCCCO2)CN(c2ccccc2)c2cc(Cl)c(-c3ccc(F)c(C(=O)O)c3)cc2S1(=O)=O]

Step 1

To a 40-mL glass vial equipped with a magnetic stir bar were charged methyl 5-((3S)-3-(1-(allyloxy)but-3-en-1-yl)-7-chloro-2-methyl-1,1-dioxido-5-phenyl-2,3,4,5-tetrahydrobenzo[f][1,2,5]thiadiazepin-8-yl)-2-fluorobenzoate (122 mg, 0.209 mmol), Grubbs Catalyst M204 (44 mg, 0.052 mmol) and DCM (10 mL); the resulting solution was sparged with nitrogen. The reaction vial was sealed, heated to 45° C. and stirred overnight, then concentrated under reduced pressure onto silica gel and purified by flash chromatography, affording methyl 5-((3S)-7-chloro-3-(3,6-dihydro-2H-pyran-2-yl)-2-methyl-1,1-dioxido-5-phenyl-2,3,4,5-tetrahydrobenzo[f][1,2,5]thiadiazepin-8-yl)-2-fluorobenzoate (61 mg, 53%) as a colorless solid. A portion of this material (27 mg, 0.048 mmol) was then charged to a 20-mL glass vial containing a magnetic stir bar; 10% Pd-C(10 mg) was added, followed by ethyl acetate (0.5 mL). The mixture was sparged with hydrogen, then stirred for 2 h under hydrogen atmosphere before being filtered and concentrated under reduced pressure. Purification by RP-HPLC afforded 5-((3S)-7-chloro-2-methyl-1,1-dioxido-5-phenyl-3-(tetrahydro-2H-pyran-2-yl)-2,3,4,5-tetrahydrobenzo[f][1,2,5]thiadiazepin-8-yl)-2-fluorobenzoic acid, Ex.104, as a colorless solid:

| Example | Structure | MS | NMR |
|---|---|---|---|
| 104 | 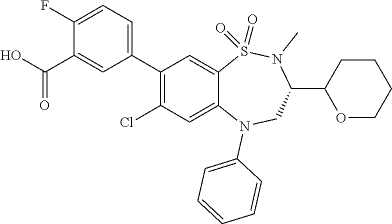 [SMILES: CN1[C@H](C2CCCCO2)CN(c2ccccc2)c2cc(Cl)c(-c3ccc(F)c(C(=O)O)c3)cc2S1(=O)=O] | [M + H]⁺ 545.4 | ¹H NMR (400 MHz, CDCl₃) δ 8.11 (dd, J = 6.9, 2.4 Hz, 1H), 7.85 (s, 1H), 7.65 (ddd, J = 8.5, 4.5, 2.5 Hz, 1H), 7.37 (t, J = 7.8 Hz, 2H), 7.28-7.19 (m, 1H), 7.17-7.02 (m, 4H), 4.35 (s, 2H), 3.91 (d, J = 11.4 Hz, 1H), 3.64 (t, J = 9.9 Hz, 1H), 3.35 (s, 2H), 2.97 (s, 3H), 2.08 (d, J = 12.9 Hz, 1H), 1.87 (s, 1H), 1.51 (s, 3H), 1.32-1.16 (m, 2H). |

### Ex.105: Synthesis of (R)-5-(7-chloro-2-methyl-1,1-dioxido-5-phenyl-3-(tetrahydro-2H-pyran-4-yl)-2,3,4,5-tetrahydrobenzo[f][1,2,5]thiadiazepin-8-yl)-2-fluorobenzoic acid

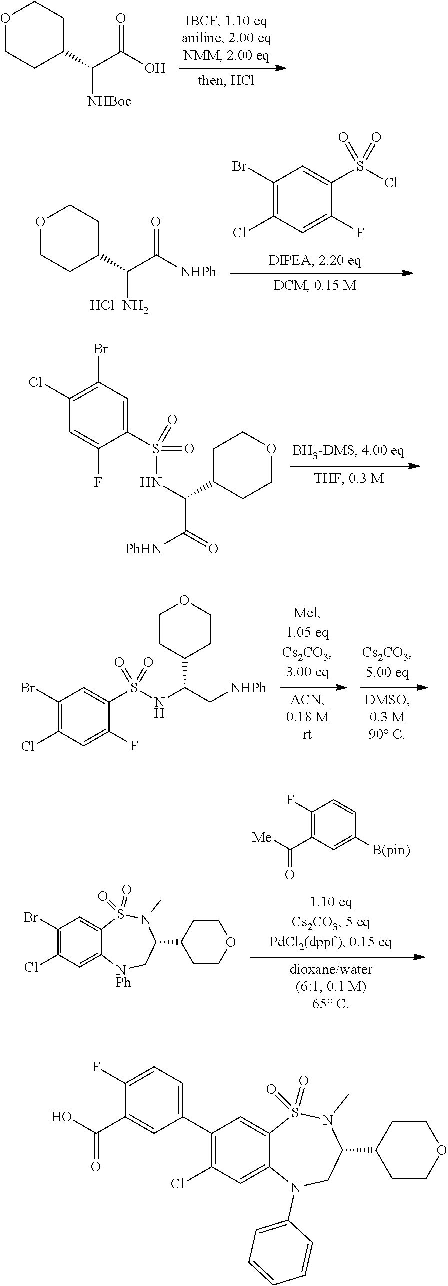

Step 1

To a 100-mL round bottom flask equipped with a magnetic stir bar and containing a colorless mixture of (R)-2-((tert-butoxycarbonyl)amino)-2-(tetrahydro-2H-pyran-4-yl)acetic acid (1.10 g, 4.24 mmol) in THF (7 mL) was added N-methylmorpholine (0.933 ml, 8.48 mmol), followed by isobutyl chloroformate (0.613 ml, 4.67 mmol). Aniline (0.775 ml, 8.48 mmol) was added, and the reaction mixture was stirred overnight, then concentrated under reduced pressure. The resulting residue was taken up in HCl (4.00 M solution in dioxane, 10.60 ml, 42.4 mmol) and stirred 1.5 h at r.t., then once again concentrated under reduced pressure to give an off-white residue. This residue was charged to a 100-mL round bottom flask equipped with a magnetic stir bar; DCM (20 mL) and Hünig's base (2.38 ml, 13.63 mmol) were added, followed by 5-bromo-4-chloro-2-fluorobenzenesulfonyl chloride (1.70 g, 5.51 mmol). The walls of the flask were rinsed with the additional DCM (8 mL) and the reaction mixture allowed to stir 36 h, at which time the reaction was diluted with water, phase-separated, then washed with 1 N HCl followed by water and concentrated under reduced pressure to reveal a brown gum. This crude sulfonamide was dissolved in THE (17 mL), giving a yellow solution, charged to a 100-mL round bottom flask, placed under nitrogen atmosphere and treated with borane-dimethyl sulfide (1.812 ml, 19.08 mmol) and heated to 55° C. After stirring for 72 h, the reaction mixture was allowed to cool to r.t. and quenched by slow addition of methanol until gas evolution ceased. The mixture was then transferred to a 100-mL round bottom flask and concentrated under reduced pressure to remove volatiles; the resulting white solid was taken up in acetonitrile (24 mL) and treated with cesium carbonate (4.14 g, 12.72 mmol). The flask was capped with a rubber septum and the resulting mixture stirred vigorously as methyl iodide (0.278 ml, 4.45 mmol) was added slowly over 30 minutes. The reaction mixture was then stirred an additional 1.5 h. Volatiles were removed under reduced pressure, and additional cesium carbonate (2.76 g, 8.48 mmol) was added, followed by DMSO (14 ml), giving a yellow mixture. The flask was then brought to 90° C. and the mixture stirred overnight; the next morning, the reaction mixture was filtered through Celite, the filter cake rinsed with DCM, and the resulting filtrates concentrated under reduced pressure then purified by flash chromatography on silica gel eluting with ethyl acetate in cyclohexane to afford (R)-8-bromo-7-chloro-2-methyl-5-phenyl-3-(tetrahydro-2H-pyran-4-yl)-2,3,4,5-tetrahydrobenzo[f][1,2,5]thiadiazepine 1,1-dioxide (0.480 g, 23%) as a white solid. ESI-MS m/z=484.9 [M+H]⁺.

Step 2

To a 2-dram vial containing (R)-8-bromo-7-chloro-2-methyl-5-phenyl-3-(tetrahydro-2H-pyran-4-yl)-2,3,4,5-tetrahydrobenzo[f][1,2,5]thiadiazepine 1,1-dioxide (100 mg, 0.206 mmol) and 2-fluoro-5-(4,4,5,5-tetramethyl-1,3,2-dioxaborolan-2-yl)benzoic acid (60.2 mg, 0.226 mmol) was added cesium carbonate (335 mg, 1.029 mmol) and a magnetic stir bar, followed by dioxane (2 mL). The mixture was sparged 5 min with nitrogen; PdCl₂(dppf) (22.59 mg, 0.031 mmol) was then added and the resulting mixture heated to 65° C. for 4.5 h. The reaction mixture was diluted with formic acid, partitioned between DCM and water, and the aqueous phase extracted with DCM. The combined organics were purified by flash chromatography on silica gel to afford (R)-5-(7-chloro-2-methyl-1,1-dioxido-5-phenyl-3-(tetrahydro-2H-pyran-4-yl)-2,3,4,5-tetrahydrobenzo[f][1,2,5]thiadiazepin-8-yl)-2-fluorobenzoic acid, Ex.105 (83 mg, 74%), as a white solid:

| Example | Structure | MS |
|---|---|---|
| 105 | 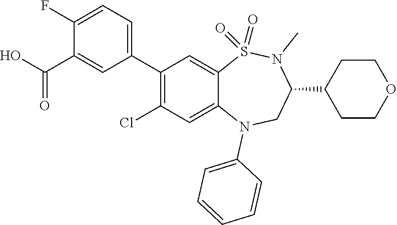 [SMILES: CN1[C@H](C2CCOCC2)CN(c2ccccc2)c2cc(Cl)c(-c3ccc(F)c(C(=O)O)c3)cc2S1(=O)=O] | [M + H]⁺ 545.3 |

The following compound was prepared in a manner identical to that described above, except that in Step 1 (R)-2-((tert-butoxycarbonyl)amino)-2-(oxetan-3-yl)acetic acid was used in place of (R)-2-((tert-butoxycarbonyl)amino)-2-(tetrahydro-2H-pyran-4-yl)acetic acid, Ex.106:

| Example | Structure | MS |
|---|---|---|
| 106 | 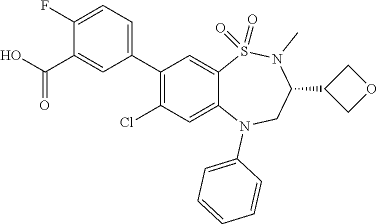 [SMILES: CN1[C@H](C2COC2)CN(c2ccccc2)c2cc(Cl)c(-c3ccc(F)c(C(=O)O)c3)cc2S1(=O)=O] | [M + H]⁺ 517.2 |

### Ex.107: Synthesis of (S)-5-(3-(bromomethyl)-7-chloro-2-methyl-1,1-dioxido-5-phenyl-2,3,4,5-tetrahydrobenzo[f][1,2,5]thiadiazepin-8-yl)-2-fluorobenzoic acid

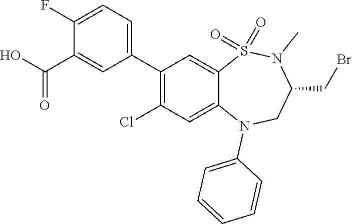 [SMILES: CN1[C@H](CBr)CN(c2ccccc2)c2cc(Cl)c(-c3ccc(F)c(C(=O)O)c3)cc2S1(=O)=O]

Step 1

To a 20-mL glass vial containing a magnetic stir bar were added methyl (S)-5-(7-chloro-3-(hydroxymethyl)-2-methyl-1,1-dioxido-5-phenyl-2,3,4,5-tetrahydrobenzo[f][1,2,5]thiadiazepin-8-yl)-2-fluorobenzoate (300 mg, 0.594 mmol), DCM (6 ml), CBr₄ (404 mg, 1.218 mmol), and polymer-supported triphenylphosphine (3 mmol/gram, 406 mg, 1.218 mmol). The resulting mixture was stirred overnight, then filtered and concentrated under reduced pressure to afford methyl (S)-5-(3-(bromomethyl)-7-chloro-2-methyl-1,1-dioxido-5-phenyl-2,3,4,5-tetrahydrobenzo[f][1,2,5]thiadiazepin-8-yl)-2-fluorobenzoate (267 mg, 79%) as an off-white solid. ESI-MS m z 566.9 [M+H]⁺.

Step 2

To a 2-dram glass vial were charged methyl (S)-5-(3-(bromomethyl)-7-chloro-2-methyl-1,1-dioxido-5-phenyl-2,3,4,5-tetrahydrobenzo[f][1,2,5]thiadiazepin-8-yl)-2-fluorobenzoate (20 mg, 0.035 mmol) and 1,4-dioxane (0.2 mL), giving a colorless solution; methanol (0.2 mL) and 3 N aqueous sodium hydroxide (0.2 mL) were added and the resulting solution stirred 40 minutes. The reaction was then quenched with acetic acid (0.05 mL) and purified by RP-HPLC to afford (S)-5-(3-(bromomethyl)-7-chloro-2-methyl-1,1-dioxido-5-phenyl-2,3,4,5-tetrahydrobenzo[f][1,2,5]thiadiazepin-8-yl)-2-fluorobenzoic acid (16 mg, 82%) as a colorless solid. ESI MS m/z=553.1 [M+H]⁺.

| Example | Structure | MS |
|---|---|---|
| 107 | 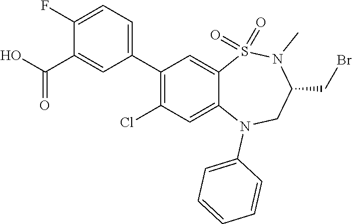 [SMILES: CN1[C@H](CBr)CN(c2ccccc2)c2cc(Cl)c(-c3ccc(F)c(C(=O)O)c3)cc2S1(=O)=O] | [M + H]⁺ 553.1 |

### Ex.108: Synthesis of(R)-5-(7-chloro-2-methyl-1,1-dioxido-5-phenyl-3-(piperidin-1-ylmethyl)-2,3,4,5-tetrahydrobenzo[f][1,2,5]thiadiazepin-8-yl)-2-fluorobenzoic acid

Step 1

To a 2-dram vial containing a magnetic stir bar, methyl (S)-5-(3-(bromomethyl)-7-chloro-2-methyl-1,1-dioxido-dioxido-5-phenyl-2,3,4,5-tetrahydrobenzo[f][1,2,5]thiadiazepin-8-yl)-2-fluorobenzoate (20 mg, 0.035 mmol), and piperidine (0.05 mL, 0.51 mmol) were added acetonitrile (0.35 mL) and cesium carbonate (50.5 mg, 0.155 mmol). The resulting mixture was stirred vigorously at rt 24 h, then concentrated and partitioned between water and ethyl acetate. The aqueous phase was extracted twice with ethyl acetate, then the combined organics were concentrated, re-constituted in dioxane (0.25 mL), methanol (0.25), and 3 N aqueous NaOH (0.25 mL), and stirred 1.5 hours before being purified by RP-HPLC to afford (R)-5-(7-chloro-2-methyl-1,1-dioxido-5-phenyl-3-(piperidin-1-ylmethyl)-2,3,4,5-tetrahydrobenzo[f][1,2,5]thiadiazepin-8-yl)-2-fluorobenzoic acid (11 mg, 56%), as a colorless solid.

| Example | Structure | MS |
|---|---|---|
| 108 | 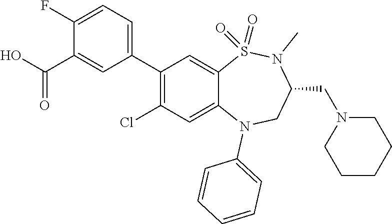 | [M + H]⁺ 517.2 |

Examples 109 to 115 were prepared by procedures identical to that described above, except that in Step 2, the appropriate amine was substituted for piperidine.

| Example | Structure | MS |
|---|---|---|
| 109 | 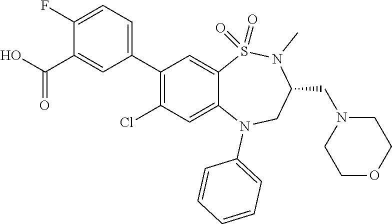 [SMILES: CN1[C@H](CN2CCOCC2)CN(c2ccccc2)c2cc(Cl)c(-c3ccc(F)c(C(=O)O)c3)cc2S1(=O)=O] | [M + H]⁺ 560.3 |
| 110 | 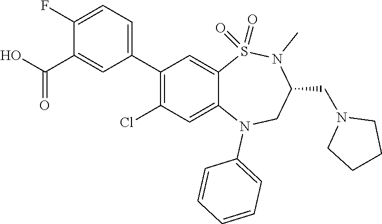 [SMILES: CN1[C@H](CN2CCCC2)CN(c2ccccc2)c2cc(Cl)c(-c3ccc(F)c(C(=O)O)c3)cc2S1(=O)=O] | [M + H]⁺ 543.9 |
| 111 | 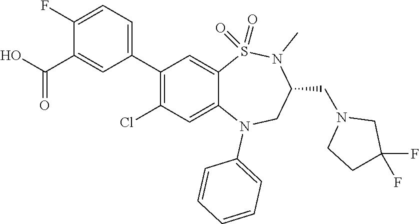 [SMILES: CN1[C@H](CN2CCC(F)(F)C2)CN(c2ccccc2)c2cc(Cl)c(-c3ccc(F)c(C(=O)O)c3)cc2S1(=O)=O] | [M + H]⁺ 580.3 |
| 112 | 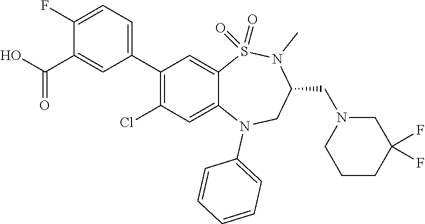 [SMILES: CN1[C@H](CN2CCCC(F)(F)C2)CN(c2ccccc2)c2cc(Cl)c(-c3ccc(F)c(C(=O)O)c3)cc2S1(=O)=O] | [M + H]⁺ 594.3 |
| 113 | 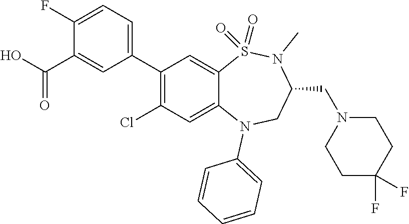 [SMILES: CN1[C@H](CN2CCC(F)(F)CC2)CN(c2ccccc2)c2cc(Cl)c(-c3ccc(F)c(C(=O)O)c3)cc2S1(=O)=O] | [M + H]⁺ 594.3 |
| 114 | 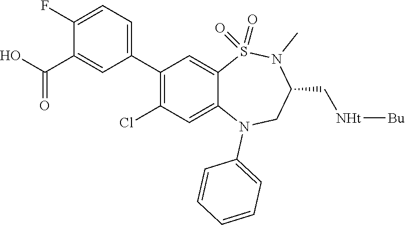 | [M + H]⁺ 546.4 |
| 115 | 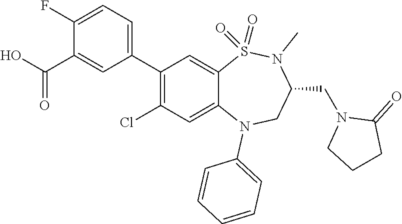 [SMILES: CN1[C@H](CN2CCCC2=O)CN(c2ccccc2)c2cc(Cl)c(-c3ccc(F)c(C(=O)O)c3)cc2S1(=O)=O] | [M + H]⁺ 558.4 |

### Ex.116: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-3-fluorothiophene-2-carboxylic acid

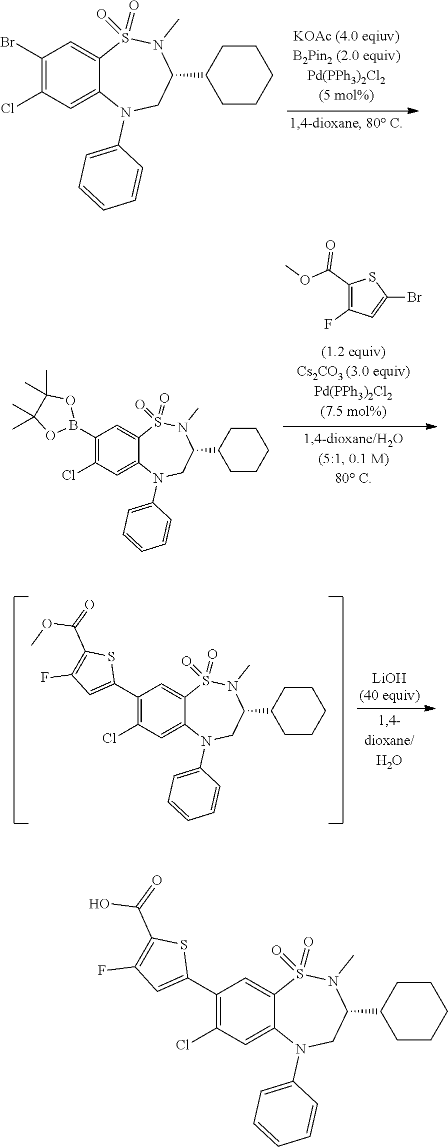

Step 1

In a 40 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (520 mg, 1.08 mmol, 1.0 equiv), bis(pinacolato)diboron (546.0 mg, 2.15 mmol, 2.0 equiv), potassium acetate (422.0 mg, 4.30 mmol, 4.0 equiv), and bis(triphenylphosphine)palladium(II) chloride (37.7 mg, 0.054 mmol, 0.05 equiv) were combined neat under a nitrogen atmosphere. Next, 1,4-dioxane (5.37 mL, 0.2M) was added, and the reaction mixture was heated at 80° C. for 12 h. Upon cooling to room temperature, analysis of the reaction mixture by LCMS indicated incomplete conversion to the desired arylboronic ester. Additional bis(pinacolato)diboron (273.0 mg, 1.08 mmol, 1.0 equiv) was added. The reaction mixture was re-heated to 80° C. for 16 h. Upon cooling to room temperature, the mixture was concentrated and purified by silica gel column chromatography (0 to 50% ethyl acetate/hexanes) to afford (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide as a white solid (350.5 mg, 0.66 mmol, 61% yield). ESI MS m/z=531.2 [M+H]⁺.

Step 2

In a 4 mL vial equipped with a stir bar, (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (30.0 mg, 0.057 mmol, 1.0 equiv), methyl 5-bromo-3-fluorothiophene-2-carboxylate (16.2 mg, 0.068 mmol, 1.2 equiv), cesium carbonate (55.2 mg, 0.170 mmol, 3.0 equiv), and bis(triphenylphosphine)palladium(II) chloride (2.97 mg, 4.24 μmol, 0.075 equiv) were combined under a nitrogen atmosphere. Next, 1,4-dioxane (0.47 mL) and water (94 μL) was added and the vial was sealed with electrical tape and heated at 80° C. for 4 h. Upon cooling to room temperature, analysis of the reaction mixture by LCMS indicated high conversion to the desired biaryl methyl ester intermediate. Lithium hydroxide (54.1 mg, 2.260 mmol, 40 equiv) was added, and the resulting mixture was stirred overnight at room temperature. Upon full conversion to the carboxylic acid, as judged by LCMS analysis, the mixture was quenched with 500 μL of formic acid, passed through a 0.45 micron syringe filter using DMIF to rinse the vial, and purified by RPHIPLC and lyophilized from MeCN/water to afford (R)-5-(7-chloro-3-cyclohexyl-2-methyl-1,1I-dioxido-5-phenyl-2,3,4,5-tetrahydrobenzo[f][1,2,5]thiadiazepin-8-yl)-3-fluorothiophene-2-carboxylic acid, Ex.116 (5.83 mg, 10.6 μmol, 1900 yield). ¹H NMR (400 MHz, CD₃CN) δ 8.00 (s, 1H), 7.42-7.38 (i, 2H), 7.31 (s, 1H), 7.20-7.07 (m, 4H), 4.30 (d, J 15.9 Hz, 1H), 3.98 (s, 1H), 3.32 (t, J=10.7 Hz, 1H), 2.83 (s, 3H), 2.10-2.00 (m, 1H), 1.87-1.66 (m, 5H), 1.34-1.14 (m, 3H), 1.08-0.95 (in, 2H). ESI MS m/z=549.0 [M+H]⁺.

Examples 117-125 were prepared using procedures analogous to that described for Ex. 116.

| Example | Structure | MS | NMR |
|---|---|---|---|
| 116 | 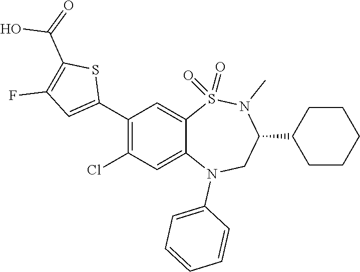 [SMILES: CN1[C@H](C2CCCCC2)CN(c2ccccc2)c2cc(Cl)c(-c3cc(F)c(C(=O)O)s3)cc2S1(=O)=O] | [M + H]⁺ 549.0 | ¹H NMR (400 MHz, CD₃CN) δ 8.00 (s, 1H), 7.42-7.38 (m, 2H), 7.31 (s, 1H), 7.20-7.07 (m, 4H), 4.30 (d, J = 15.9 Hz, 1H), 3.98 (s, 1H), 3.32 (t, J = 10.7 Hz, 1H), 2.83 (s, 3H), 2.10-2.00 (m, 1H), 1.87- 1.66 (m, 5H), 1.34-1.14 (m, 3H), 1.08-0.95 (m, 2H) |
| 117 | 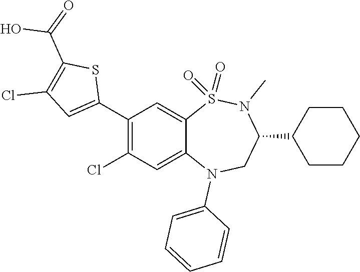 [SMILES: CN1[C@H](C2CCCCC2)CN(c2ccccc2)c2cc(Cl)c(-c3cc(Cl)c(C(=O)O)s3)cc2S1(=O)=O] | [M + H]⁺ 565.0 | |
| 118 | 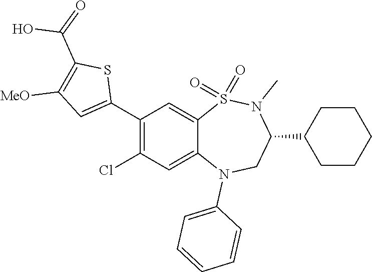 [SMILES: COc1cc(-c2cc3c(cc2Cl)N(c2ccccc2)C[C@@H](C2CCCCC2)N(C)S3(=O)=O)sc1C(=O)O] | [M + H]⁺ 561.0 | ¹H NMR (400 MHz, CD₃CN) δ 8.05 (s, 1H), 7.41-7.37 (m, 2H), 7.34 (s, 1H), 7.13-7.11 (m, 4H), 4.32 (d, J = 15.9 Hz, 1H), 4.06 (s, 3H), 3.95 (s, 1H), 3.34 (t, J = 10.5 Hz, 1H), 2.82 (s, 4H), 2.05 (d, J = 13.2 Hz, 1H), 1.88 (d, J = 12.5 Hz, 1H), 1.82-1.61 (m, 4H), 1.35- 1.15 (m, 3H), 1.08-0.95 (m, 2H). |
| 119 | 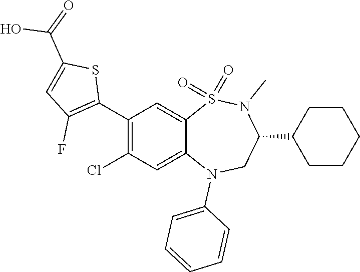 [SMILES: CN1[C@H](C2CCCCC2)CN(c2ccccc2)c2cc(Cl)c(-c3sc(C(=O)O)cc3F)cc2S1(=O)=O] | [M − H]⁻ 547.0 | |
| 120 | 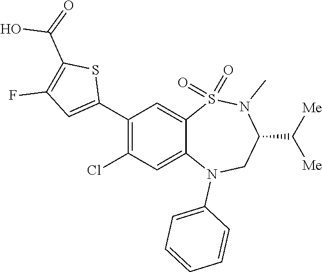 [SMILES: CC(C)[C@@H]1CN(c2ccccc2)c2cc(Cl)c(-c3cc(F)c(C(=O)O)s3)cc2S(=O)(=O)N1C] | [M + H]⁺ 509.0 | ¹H NMR (400 MHz, CD₃CN) δ 9.70 (s, 1H), 8.01 (s, 1H), 7.43-7.39 (m, 2H), 7.31 (s, 1H), 7.16-7.11 (m, 4H), 4.30 (d, J = 16.0 Hz, 1H), 4.01 (s, 1H), 3.25 (t, J = 10.7 Hz, 1H), 2.85 (s, 3H), 2.25-2.10 (m, 1H), 1.07 (d, J = 6.6 Hz, 3H), 1.01 (d, J = 6.6 Hz, 3H). |
| 121 | 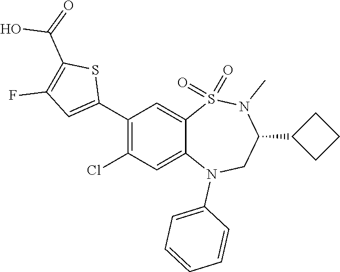 [SMILES: CN1[C@H](C2CCC2)CN(c2ccccc2)c2cc(Cl)c(-c3cc(F)c(C(=O)O)s3)cc2S1(=O)=O] | [M + H]⁺ 521.1 | ¹H NMR (400 MHz, CD₃CN) δ 8.07 (s, 1H), 7.37-7.33 (m, 4H), 7.04-6.96 (m, 3H), 4.02 (d, J = 15.9 Hz, 1H), 3.77 (t, J = 10.6 Hz, 1H), 3.60-3.37 (m, 1H), 2.67 (s, 3H), 2.59- 2.52 (m, 1H), 2.18-2.09 (m, 3H), 1.93-1.79 (m, 3H). |
| 122 | 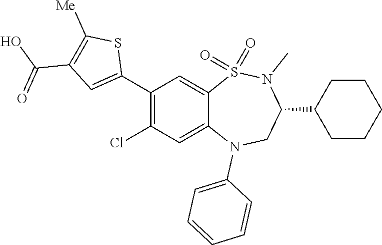 [SMILES: Cc1sc(-c2cc3c(cc2Cl)N(c2ccccc2)C[C@@H](C2CCCCC2)N(C)S3(=O)=O)cc1C(=O)O] | [M + H]⁺ 545.0 | |
| 123 | 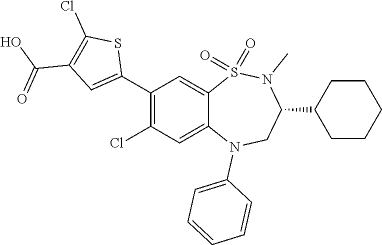 [SMILES: CN1[C@H](C2CCCCC2)CN(c2ccccc2)c2cc(Cl)c(-c3cc(C(=O)O)c(Cl)s3)cc2S1(=O)=O] | [M + H]⁺ 565.0 | |
| 124 | 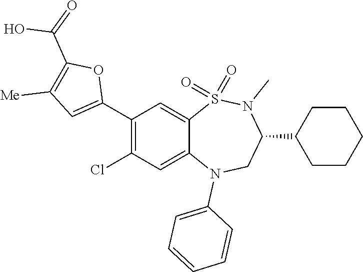 [SMILES: Cc1cc(-c2cc3c(cc2Cl)N(c2ccccc2)C[C@@H](C2CCCCC2)N(C)S3(=O)=O)oc1C(=O)O] | [M + H]⁺ 529.1 | |
| 125 | 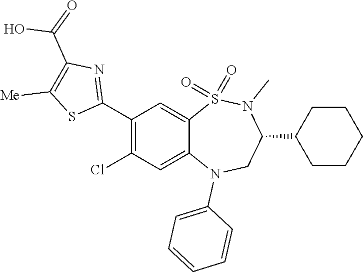 [SMILES: Cc1sc(-c2cc3c(cc2Cl)N(c2ccccc2)C[C@@H](C2CCCCC2)N(C)S3(=O)=O)nc1C(=O)O] | [M + H]⁺ 546.2 | |

### Ex.126: Synthesis of(R)-5-(7-chloro-3-cyclohexyl-2-methyl-1,1-dioxido-5-phenyl-2,3,4,5-tetrahydrobenzo[f][1,2,5]thiadiazepin-8-yl)-3-methylthiophene-2-carboxylic acid

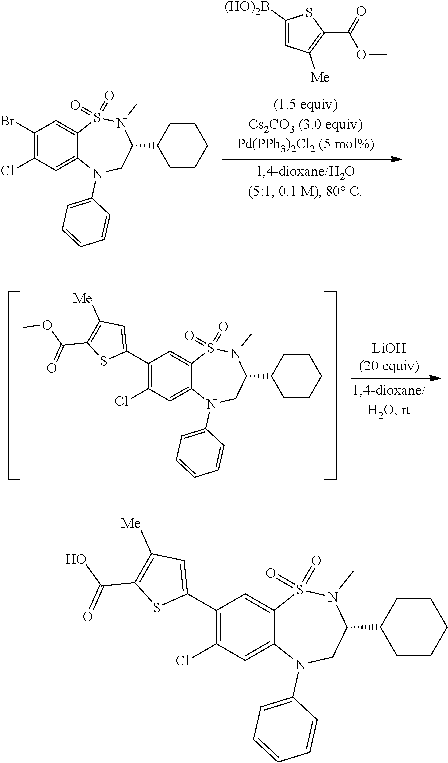

Step 1

In a 4 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide was combined neat with (5-(methoxycarbonyl)-4-methylthiophen-2-yl)boronic acid (18.6 mg, 0.093 mmol, 1.5 equiv, CAS #1256345-70-6), cesium carbonate (60.6 mg, 0.186 mmol, 3.0 equiv), and bis(triphenylphosphine)palladium(II) chloride (2.18 mg, 5 mol %) under a nitrogen atmosphere. Next, 1,4-dioxane (0.52 mL) and water (0.10 mL) were added and the vial was sealed with electrical tape. The reaction was heated at 80° C. for 16 h. Upon cooling to room temperature, LCMS analysis indicated the formation of methyl (R)-5-(7-chloro-3-cyclohexyl-2-methyl-1,1-dioxido-5-phenyl-2,3,4,5-tetrahydrobenzo[f][1,2,5]thiadiazepin-8-yl)-3-methylthiophene-2-carboxylate.

Step 2

To the reaction mixture containing methyl (R)-5-(7-chloro-3-cyclohexyl-2-methyl-1,1-dioxido-5-phenyl-2,3,4,5-tetrahydrobenzo[f][1,2,5]thiadiazepin-8-yl)-3-methylthiophene-2-carboxylate generated above was added lithium hydroxide (29.7 mg, 20 equiv). The mixture was stirred for 8 h, and additional lithium hydroxide (29.7 mg, 20 equiv) was added. The mixture was stirred for an additional 16 h and then quenched with formic acid (0.25 mL). Purification by RPHPLC and lyophilization of the isolated material from acetonitrile and water afforded (R)-5-(7-chloro-3-cyclohexyl-2-methyl-1,1-dioxido-5-phenyl-2,3,4,5-tetrahydrobenzo[f][1,2,5]thiadiazepin-8-yl)-3-methylthiophene-2-carboxylic acid, Ex.126 (2.08 mg, 6.1% yield). ESI MS m/z=545.0 [M+H]⁺.

| Example | Structure | MS |
|---|---|---|
| 126 | 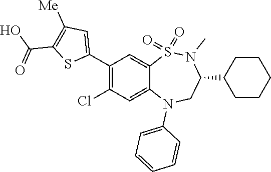 [SMILES: Cc1cc(-c2cc3c(cc2Cl)N(c2ccccc2)C[C@@H](C2CCCCC2)N(C)S3(=O)=O)sc1C(=O)O] | [M + H]⁺ 545.0 |

### Ex.127: Synthesis of methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate

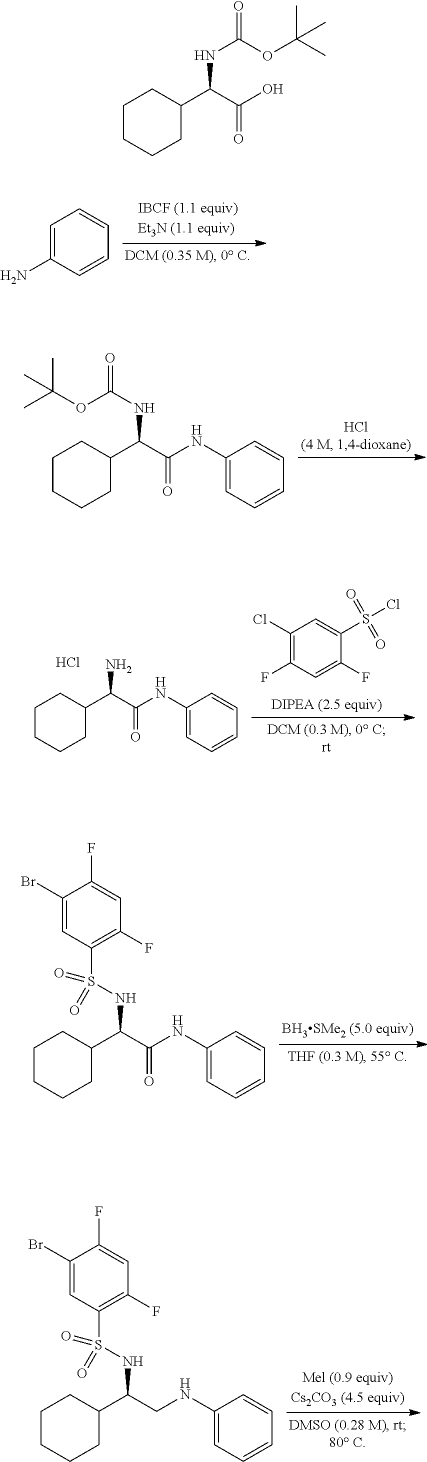

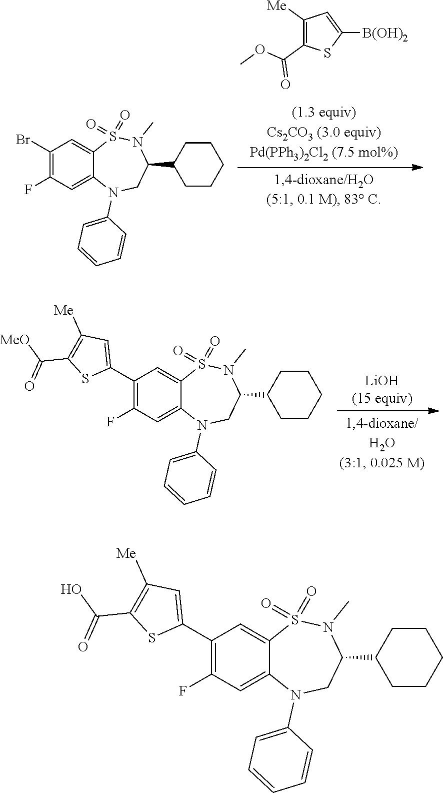 [SMILES: COC(=O)c1sc(B(O)O)cc1C ; CN1[C@@H](C2CCCCC2)CN(c2ccccc2)c2cc(F)c(Br)cc2S1(=O)=O ; COC(=O)c1sc(-c2cc3c(cc2F)N(c2ccccc2)C[C@@H](C2CCCCC2)N(C)S3(=O)=O)cc1C ; Cc1cc(-c2cc3c(cc2F)N(c2ccccc2)C[C@@H](C2CCCCC2)N(C)S3(=O)=O)sc1C(=O)O]

Step 1

In a 1L round bottom flask equipped with a stir bar, (R)-2-((tert-butoxycarbonyl)amino)-2-cyclohexylacetic acid (25.04 g, 97 mmol, 1.0 equiv, CAS #70491-05-3) was dissolved in methylene chloride (278 mL, 0.35M) under a nitrogen atmosphere. The solution was cooled to 0° C. using an ice/water bath. Next, isobutyl chloroformate (14.1 mL, 14.62 g, 107 mmol, 1.1 equiv) was added, followed by triethylamine (14.9 mL, 10.83 g, 107 mmol, 1.1 equiv). The resulting mixture was stirred for 30 minutes at 0° C. prior to the addition of aniline (9.8 mL, 9.97 g, 107 mmol). The resulting mixture was stirred for 16 h while being allowed to warm to room temperature. At this time, LCMS indicated full conversion to the anilide and the mixture was diluted with DCM (300 mL) and washed twice with 1.2M HCl (300 mL each) and once with brine. The organic layer was dried over magnesium sulfate, filtered through celite using methylene chloride to rinse, and concentrated to afford crude tert-butyl (R)-(1-cyclohexyl-2-oxo-2-(phenylamino)ethyl)carbamate (32.4 grams theoretical) which was used in the next step without purification. ESI MS m/z=355.2 [M+Na]*.

Step 2

The crude tert-butyl (R)-(1-cyclohexyl-2-oxo-2-(phenylamino)ethyl)carbamate (32.4 grams theoretical) obtained in Step 1 was dissolved in 4M HCl in 1,4-dioxane (200 mL, 8.85 equiv). Upon full conversion to the hydrochloride salt, as judged by LCMS analysis, the reaction mixture was concentrated in vacuo to afford (R)-2-amino-2-cyclohexyl-N-phenylacetamide hydrochloride (24.25 g theoretical) as a white solid that was used without further purification in the next stage. ESI MS m/z=233.0 [M+H]⁺.

Step 3

The crude (R)-2-amino-2-cyclohexyl-N-phenylacetamide hydrochloride (24.25 g theoretical) obtained in Step 3 was suspended in methylene chloride (301 mL, 0.3M) and the flask was cooled to 0° C. Next, N,N-diisopropylethylamine (39.4 mL, 29.2 g, 226 mmol) was added resulting in a homogenous solution. Following the addition of 5-chloro-2,4-difluorobenzenesulfonyl chloride (26.3 g, 90 mmol, 1.0 equiv, CAS #287172-61-6) the reaction was allowed to reach room temperature and was stirred for 20 h. Upon full conversion to the desired sulfonamide, as judged by LCMS analysis of the reaction mixture, the mixture was diluted with 300 mL of 1.2M HCl and the aqueous phase was extracted three times with methylene chloride (100 mL). The combined organic layers were washed three times with 1.2M HCl (100 mL each) and dried over magnesium sulfate. Filtration and concentration of the reaction mixture afforded the crude (R)-2-((5-bromo-2,4-difluorophenyl)sulfonamido)-2-cyclohexyl-N-phenylacetamide (44.0 g theoretical) which was used in the next step without purification. ESI MS m/z=487.0 [M+H]⁺.

Step 4

In a 1 L round bottom flask equipped with a stir bar and a reflux condenser, (R)-2-((5-bromo-2,4-difluorophenyl)sulfonamido)-2-cyclohexyl-N-phenylacetamide (44.0 g theoretical) was dissolved in THE (301 mL, 0.3M) under a nitrogen atmosphere. Next, borane dimethyl sulfide complex (42.9 mL, 34.3 g, 451 mmol, 5 equiv) was added, and the mixture was heated for 19 h at 55° C. Upon completion the reaction was cooled in an ice bath, and was carefully and slowly quenched with 100 mL of water. The mixture was further diluted with water (300 mL) and extracted three times with ethyl acetate (150 mL each). The combined organic layers were dried over magnesium sulfate, filtered through celite and concentrated to afford the crude (R)-5-bromo-N-(1-cyclohexyl-2-(phenylamino)ethyl)-2,4-difluorobenzenesulfonamide (40.32 g, 94% yield) that was used without purification in the subsequent step. ESI MS m/z=473.0 [M+H]⁺.

Step 5

In a 1 L round bottom flask equipped with a stir bar and a reflux condenser, R)-5-bromo-N-(1-cyclohexyl-2-(phenylamino)ethyl)-2,4-difluorobenzenesulfonamide (40.32 g) was dissolved in dimethyl sulfoxide (300 mL, 0.28M) under a nitrogen atmosphere. Cesium carbonate (125 g, 383 mmol, 4.5 equiv) was added, followed by iodomethane (4.79 mL, 10.88 g, 77 mmol, 0.9 equiv). After 30 minutes, LCMS analysis indicated incomplete methylation of the sulfonamide and additional iodomethane (0.53 mL, 1.209 g, 8.5 mmol, 0.1 equiv) was added. Upon full conversion to the methyl sulfonamide intermediate, as judged by LCMS analysis, the reaction was heated at 80° C. for 24 h. Upon cooling to room temperature the reaction was diluted with water (500 mL). The aqueous phase was extracted three times with ethyl acetate (200 mL each) and the combined organic layers were dried over magnesium sulfate. Upon filtration through celite and concentration, the crude residue was purified by silica gel column chromatography (gradient elution, 0 to 20% ethyl acetate in cyclohexane) to afford (R)-8-bromo-3-cyclohexyl-7-fluoro-2-methyl-5-phenyl-2,3,4,5-tetrahydrobenzo[f][1,2,5]thiadiazepine 1,1-dioxide (23.05 g, 58% yield). ESI MS m/z=467.0 [M+H]⁺.

Step 6

In a 40 mL vial equipped with a stir bar, (R)-8-bromo-3-cyclohexyl-7-fluoro-2-methyl-5-phenyl-2,3,4,5-tetrahydrobenzo[f][1,2,5]thiadiazepine 1,1-dioxide (335.0 mg, 0.717 mmol, 1.0 equiv), (5-(methoxycarbonyl)-4-methylthiophen-2-yl)boronic acid (186.0 mg, 0.932 mmol, 1.3 equiv, CAS#1256345-70-6), cesium carbonate (701.0 mg, 2.15 mmol, 3.0 equiv), and bis(triphenylphosphine)palladium(II) chloride (37.7 mg, 0.054 mmol, 7.5 mol %) were combined neat under a nitrogen atmosphere. Next, 1,4-dioxane (5.97 mL) and water (1.20 mL) were added, and the vial was sealed with electrical tape. The reaction mixture was heated at 83° C. for 16 h. Upon cooling to room temperature, the reaction mixture was concentrated and purified by silica gel column chromatography (gradient elution, 0 to 30% ethyl acetate in cyclohexane) to afford methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate as a light yellow solid in quantitative yield (389.0 mg). ESI MS m/z=543.1 [M+H]⁺.

Step 7

In a 1 dram vial equipped with a stir bar, (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate (15.0 mg, 0.028 mmol, 1.0 equiv) was dissolved in 1,4-dioxane (0.83 mL) and water (0.28 mL). Lithium hydroxide (9.9 mg, 0.415 mmol, 15.0 equiv) was added, and the reaction mixture was stirred for 32 h. Upon completion, the reaction mixture was quenched with formic acid (300 μL), passed through a 0.45 micron syringe filter using 1.0 mL of N,N-dimethylformamide to rinse, and purified by RPHPLC. The desired compound obtained upon purification was lyophilized from acetonitrile and water to afford (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylic acid, Ex.127 (8.32 mg, 57% yield). ESI MS m/z=529.2 [M+H]⁺. ¹H NMR (400 MHz, DMSO) δ 13.11 (s, 1H), 8.15 (d, J=8.5 Hz, 1H), 7.58 (s, 1H), 7.39 (t, J=7.8 Hz, 2H), 7.15 (br s, 2H), 7.10-7.08 (m, 1H), 6.97 (s, 1H), 4.38 (d, J=16.0 Hz, 1H), 3.90 (s, 1H), 3.28 (s, 1H), 2.79 (s, 3H), 1.99-1.91 (m, 2H), 1.74-1.64 (m, 4H), 1.29-1.14 (m, 3H), 1.05-0.93 (m, 2H).

| Example | Structure | MS | NMR |
|---|---|---|---|
| 127 | 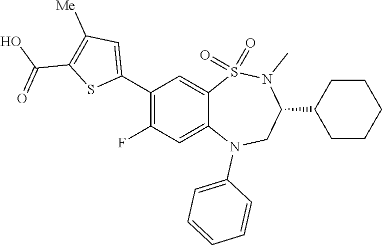 [SMILES: Cc1cc(-c2cc3c(cc2F)N(c2ccccc2)C[C@@H](C2CCCCC2)N(C)S3(=O)=O)sc1C(=O)O] | [M + H]⁺ 529.2 | ¹H NMR (400 MHz, DMSO) δ 13.11 (s, 1H), 8.15 (d, J = 8.5 Hz, 1H), 7.58 (s, 1H), 7.39 (t, J = 7.8 Hz, 2H), 7.15 (br s, 2H), 7.10 − 7.08 (m, 1H), 6.97 (s, 1H), 4.38 (d, J = 16.0 Hz, 1H), 3.90 (s, 1H), 3.28 (s, 1H), 2.79 (s, 3H), 1.99 − 1.91 (m, 2H), 1.74 − 1.64 (m, 4H), 1.29 − 1.14 (m, 3H), 1.05 − 0.93 (m, 2H) |

### Ex. 128: Synthesis of (R)-5-(3-cyclohexyl-7-fluoro-2-methyl-1,1-dioxido-5-phenyl-2,3,4,5-tetrahydrobenzo[f][1,2,5]thiadiazepin-8-yl)-3-fluorothiophene-2-carboxylic acid

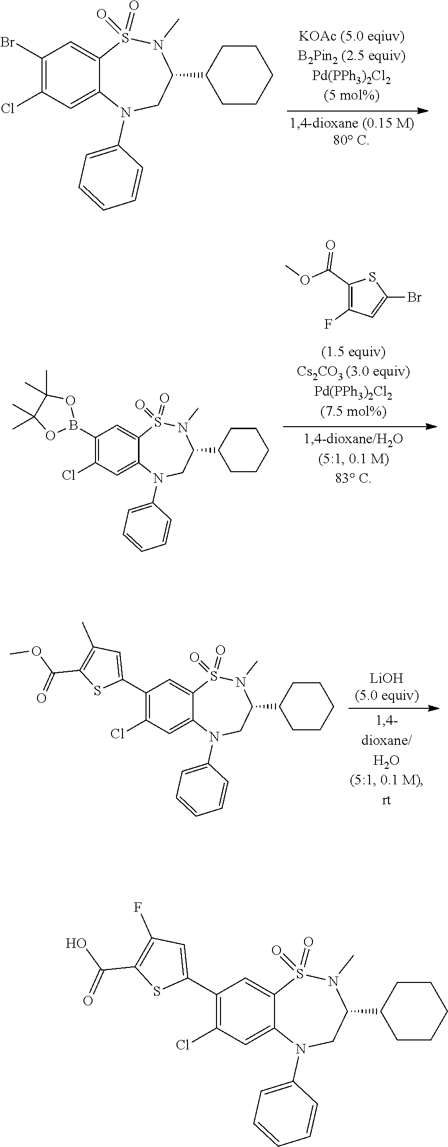 [SMILES: CN1[C@H](C2CCCCC2)CN(c2ccccc2)c2cc(Cl)c(Br)cc2S1(=O)=O ; COC(=O)c1sc(Br)cc1F ; CN1[C@H](C2CCCCC2)CN(c2ccccc2)c2cc(Cl)c(B3OC(C)(C)C(C)(C)O3)cc2S1(=O)=O ; COC(=O)c1sc(-c2cc3c(cc2Cl)N(c2ccccc2)C[C@@H](C2CCCCC2)N(C)S3(=O)=O)cc1C ; CN1[C@H](C2CCCCC2)CN(c2ccccc2)c2cc(Cl)c(-c3cc(F)c(C(=O)O)s3)cc2S1(=O)=O]

Step 1

In a 40 mL vial equipped with a stir bar, (R)-8-bromo-3-cyclohexyl-7-fluoro-2-methyl-5-phenyl-2,3,4,5-tetrahydrobenzo[f][1,2,5]thiadiazepine 1,1-dioxide (750 mg, 1.61 mmol, 1.0 equiv) was combined neat with bis(pinacolato)diboron (1.02 g, 4.01 mmol, 2.5 equiv), potassium acetate (787 mg, 8.02 mmol, 5.0 equiv), and bis(triphenylphosphine)palladium(II) chloride (84.0 mg, 0.120 mmol, 7.5 mol %) under a nitrogen atmosphere. Next, 1,4-dioxane (10.7 mL, 0.15M) was added and the vial was sealed with electrical tape and heated at 83° C. for 21 h. Upon cooling to room temperature the mixture was concentrated and purified by silica gel column chromatography (cyclohexane/ethyl acetate) to afford (R)-3-cyclohexyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (785.8 mg, 95% yield). ESI MS m/z=515.2 [M+H]⁺.

Step 2

In a 40 mL vial equipped with a stir bar, (R)-3-cyclohexyl-7-fluoro-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (785.8 mg, 1.53 mmol, 1.0 equiv), methyl 5-bromo-3-fluorothiophene-2-carboxylate (548.0 mg, 2.29 mmol, 1.5 equiv, CAS #1820885-11-7), cesium carbonate (1.49 g, 4.58 mmol, 3.0 equiv), and bis(triphenylphosphine)palladium(II) chloride (80 mg, 7.5 mol %) were combined neat under a nitrogen atmosphere. Next, 1,4-dioxane (8.49 mL) and water (1.70 mL) were added and the vial was sealed with electrical tape. The mixture was heated at 83° C. for 3.5 h. Upon cooling to room temperature, the reaction mixture was concentrated and the residue was purified by silica gel column chromatography to afford methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-fluorothiophene-2-carboxylate (463.0 mg, 56% yield).

Step 3

Methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-fluorothiophene-2-carboxylate (342.0 mg, 0.63 mmol, 1.0 equiv) was dissolved in a mixture of 1,4-dioxane (5.2 mL) and water (1.0 mL). Lithium hydroxide (74.9 mg, 3.13 mmol, 5.0 equiv) was then added and the mixture was stirred for 48 h at room temperature. The reaction mixture was quenched with 1.2M HCl (5.2 mL, 10.0 equiv), and the aqueous phase was extracted with ethyl acetate. The combined organic layers were concentrated to afford (R)-5-(3-cyclohexyl-7-fluoro-2-methyl-1,1-dioxido-5-phenyl-2,3,4,5-tetrahydrobenzo[f][1,2,5]thiadiazepin-8-yl)-3-fluorothiophene-2-carboxylic acid, Ex.128 (325.0 mg, 98% yield). ESI MS m/z=533.2 [M+H]⁺.

| Example | Structure | MS |
|---|---|---|
| 128 | 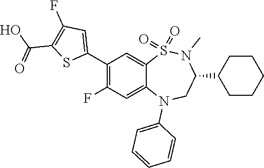 [SMILES: CN1[C@H](C2CCCCC2)CN(c2ccccc2)c2cc(F)c(-c3cc(F)c(C(=O)O)s3)cc2S1(=O)=O] | [M + H]⁺ 533.2 |

### Ex.129: Synthesis of(R)-5-(7-chloro-3-cyclohexyl-2-methyl-1,1-dioxido-5-phenyl-2,3,4,5-tetrahydrobenzo[f][1,2,5]thiadiazepin-8-yl)thiophene-3-carboxylic acid

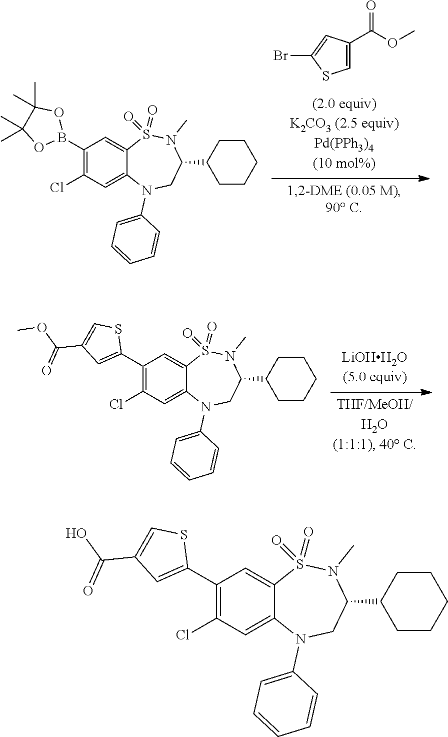

Step 1

A mixture of compound (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.19 mmol, 1.0 eq), methyl 5-bromothiophene-3-carboxylate (83.0 mg, 0.38 mmol, 2.0 eq), K₂CO₃ (64.0 mg, 0.47 mmol, 2.5 eq) and Pd(PPh₃)₄ (23.1 mg, 0.02 mmol, 0.1 eq) in DME (4 mL) was stirred for 4 h at 90° C. under nitrogen atmosphere. The resulting mixture was cooled to room temperature. The reaction was quenched with H₂O (15 mL) and extracted three times with EtOAc (15 mL each). The combined organic layers were dried over anhydrous Na₂SO₄ and concentrated. The residue was purified by silica gel column chromatography (eluting with EtOAc/Petroleum ether=1/10) to give methyl (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-3-carboxylate (100.0 mg, 97% yield) as a yellow solid. ESI MS m/z=544.9 [M+H]⁺.

Step 2

A mixture of methyl (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-3-carboxylate (100.0 mg, 0.18 mmol, 1.0 eq) and LiOH·H₂O (40.0 mg, 0.95 mmol, 5.0 eq) in MeOH (1.0 mL), H₂O (1.0 mL) and THE (1.0 mL) was stirred for 2 h at 40° C. The resulting mixture was concentrated under vacuum. The residue was adjusted to pH 6 with HCl (1 N). The reaction mixture was filtered through a pad of Celite. The residue was then purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-3-carboxylic acid, Ex.129 (30.6 mg, 32% yield) as a white solid. ESI MS m/z=531.0 [M+H]⁺. ¹H NMR (300 MHz, DMSO-d6): δ 8.33 (s, 1H), 7.90 (s, 1H), 7.69 (d, J=1.2 Hz, 1H), 7.34 (t, J=7.8 Hz, 2H), 7.20 (s, 1H), 7.12-6.98 (m, 3H), 4.44-4.28 (m, 1H), 3.77 (m, 1H), 3.43-3.30 (m, 1H), 2.73 (s, 3H), 1.99-1.87 (m, 2H), 1.78-1.53 (m, 4H), 1.25-1.14 (m, 3H), 1.03-0.89 (m, 2H).

| Example | Structure | MS | NMR |
|---|---|---|---|
| 129 | 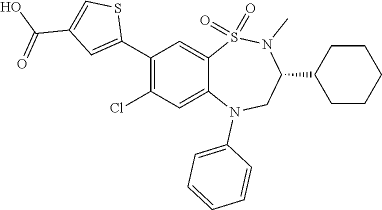 [SMILES: CN1[C@H](C2CCCCC2)CN(c2ccccc2)c2cc(Cl)c(-c3cc(C(=O)O)cs3)cc2S1(=O)=O] | [M + H]⁺ 531.0 | ¹H NMR (300 MHz, DMSO- d₆): δ 8.33 (s, 1H), 7.90 (s, 1H), 7.69 (d, J = 1.2 Hz, 1H), 7.34 (t, J = 7.8 Hz, 2H), 7.20 (s, 1H), 7.12 − 6.98 (m, 3H), 4.44 − 4.28 (m, 1H), 3.77 (m, 1H), 3.43 − 3.30 (m, 1H), 2.73 (s, 3H), 1.99 − 1.87 (m, 2H), 1.78 − 1.53 (m, 4H), 1.25 − 1.14 (m, 3H), 1.03 − 0.89 (m, 2H) |

### Ex.130: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylic acid

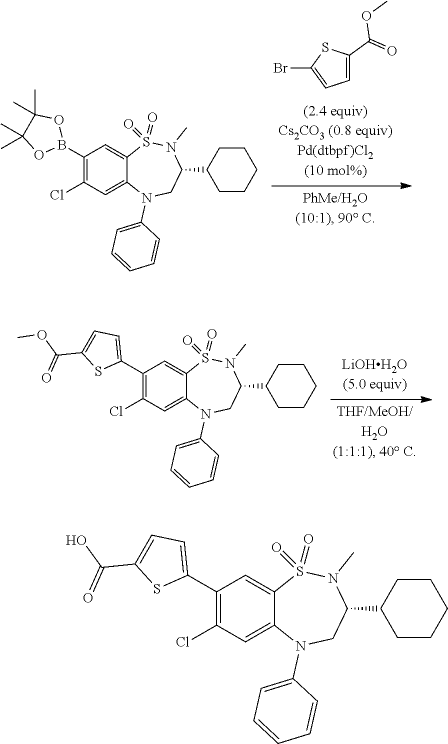

Step 1

A mixture of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (200.0 mg, 0.38 mmol, 1.0 eq), methyl 5-bromothiophene-2-carboxylate (200.0 mg, 0.91 mmol, 2.4 eq), Pd(dtbpf)Cl₂ (24.5 mg, 0.04 mmol, 0.1 eq) and Cs₂CO₃ (100.0 mg, 0.31 mmol, 0.8 eq) in toluene (5.0 mL) and H₂O (0.5 mL) was stirred for 2 h at 90° C. under nitrogen atmosphere. The resulting mixture was cooled to room temperature and quenched with H₂O (50 mL). The mixture was extracted three times with EtOAc (50 mL each), the combined organic layers were dried over anhydrous Na₂SO₄, filtered and concentrated under reduced pressure. The residue was purified by silica gel column chromatography (eluting with EtOAc/Petroleum ether=1/10) to afford methyl (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate (120.0 mg, 58% yield) as a white solid. ESI MS m/z=545.2 [M+H]⁺.

Step 2

A mixture of methyl (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate (80.0 mg, 0.15 mmol, 1.0 eq) and LiOH H₂O (31.0 mg, 0.74 mmol, 5.0 eq) in MeOH (1.0 mL), H₂O (1.0 mL) and THE (1.0 mL) was stirred for 3 h at room temperature. The resulting mixture was concentrated under vacuum, and the aqueous phase was adjusted to pH 6 with HCl (1 N) before being extracted with EtOAc (15 mL×3).

The combined organic layers were dried over anhydrous Na₂SO₄ and concentrated under reduced pressure. Then the residue was purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylic acid, Ex.130 (32.0 mg, 41% yield) as a yellow solid. ESI MS m/z=531.1 [M+H]⁺. ¹H NMR (300 MHz, CDCl₃): δ 8.07 (s, 1H), 7.86 (d, J=4.2 Hz, 1H), 7.42-7.33 (m, 3H), 7.18-7.10 (m, 3H), 7.01 (s, 1H), 4.33-4.19 (m, 2H), 3.16-3.05 (m, 1H), 2.92 (s, 3H), 2.24-2.10 (m, 1H), 1.80-1.64 (m, 5H), 1.19-1.05 (m, 2H), 0.96-0.81 (m, 3H).

| Example | Structure | MS | NMR |
|---|---|---|---|
| 130 | 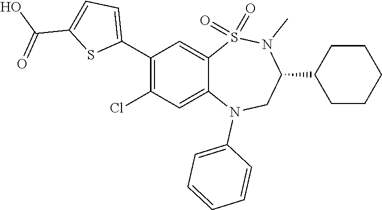 [SMILES: CN1[C@H](C2CCCCC2)CN(c2ccccc2)c2cc(Cl)c(-c3ccc(C(=O)O)s3)cc2S1(=O)=O] | [M + H]⁺ 531.1 | ¹H NMR (300 MHz, CDCl₃): δ 8.07 (s, 1H), 7.86 (d, J = 4.2 Hz, 1H), 7.42 − 7.33 (m, 3H), 7.18 − 7.10 (m, 3H), 7.01 (s, 1H), 4.33 − 4.19 (m, 2H), 3.16 − 3.05 (m, 1H), 2.92 (s, 3H), 2.24 − 2.10 (m, 1H), 1.80 − 1.64 (m, 5H), 1.19 − 1.05 (m, 2H), 0.96 − 0.81 (m, 3H) |

### Ex.131: Synthesis of (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-4-carboxylic acid

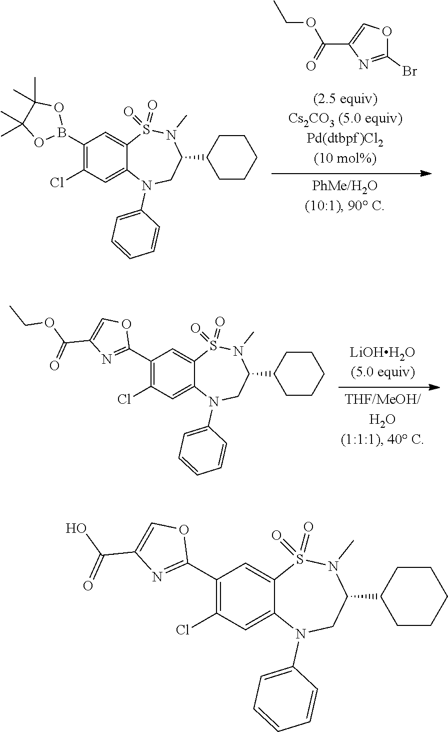 [SMILES: CCOC(=O)c1coc(Br)n1 ; CN1[C@H](C2CCCCC2)CN(c2ccccc2)c2cc(Cl)c(B3OC(C)(C)C(C)(C)O3)cc2S1(=O)=O ; CCOC(=O)c1coc(-c2cc3c(cc2Cl)N(c2ccccc2)C[C@@H](C2CCCCC2)N(C)S3(=O)=O)n1 ; CN1[C@H](C2CCCCC2)CN(c2ccccc2)c2cc(Cl)c(-c3nc(C(=O)O)co3)cc2S1(=O)=O]

Step 1

A mixture of ethyl 2-bromooxazole-4-carboxylate (100.0 mg, 0.45 mmol, 2.5 eq), (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.19 mmol, 1.0 eq), Cs₂CO₃ (306.0 mg, 0.94 mmol, 5.0 eq), Pd(dtbpf)Cl₂ (20.0 mg, 0.03 mmol, 0.2 eq) in toluene (3.0 mL) and H₂O (0.3 mL) were stirred at 90° C. for 2 h under nitrogen atmosphere. The resulting mixture was cooled to room temperature. The reaction was quenched with H₂O (50 mL) and extracted three times with EtOAc (50 mL each). The combined organic layers were dried over anhydrous Na₂SO₄, filtered, and concentrated under reduced pressure. The residue was purified by silica gel column chromatography (EtOAc/Petroleum ether=3:7) to afford ethyl (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-4-carboxylate (90.0 mg, 88% yield) as a yellow solid. ESI MS m/z=544.1 [M+H]⁺.

Step 2

A mixture of ethyl (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-4-carboxylate (90.0 mg, 0.16 mmol, 1.0 eq) and LiOH—H₂O (35.0 mg, 0.83 mmol, 5.0 eq) in MeOH (0.5 mL), THE (0.5 mL) and H₂O (0.5 mL) was stirred for 2 h at 40° C. The resulting mixture was concentrated under vacuum. The aqueous phase was adjusted to pH 6 with HCl (1N) and extracted three times with EtOAc (20 mL each). The combined organic layers were dried over anhydrous Na₂SO₄ and concentrated under reduced pressure. The residue was purified by RPHPLC to afford (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-4-carboxylic acid, Ex.131 (17.0 mg, 20% yield) as a white solid. ESI MS m/z=516.0 [M+H]⁺. ¹H NMR (400 MHz, DMSO-d6): δ 13.27 (s, 1H), 8.90 (s, 1H), 8.34 (s, 1H), 7.42 (t, J=7.6 Hz, 2H), 7.37-7.10 (m, 3H), 6.96 (s, 1H), 4.45-4.24 (m, 1H), 4.24-3.92 (m, 1H), 3.45-3.10 (m, 1H), 2.84 (s, 3H), 2.09-1.90 (m, 1H), 1.88-1.47 (m, 5H), 1.40-0.70 (m, 5H).

| Example | Structure | MS | NMR |
|---|---|---|---|
| 131 | 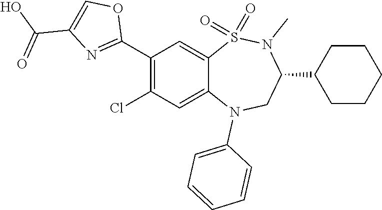 [SMILES: CN1[C@H](C2CCCCC2)CN(c2ccccc2)c2cc(Cl)c(-c3nc(C(=O)O)co3)cc2S1(=O)=O] | [M + H]⁺ 516.0 | ¹H NMR (400 MHz, DMSO-d₆): δ 13.27 (s, 1H), 8.90 (s, 1H), 8.34 (s, 1H), 7.42 (t, J = 7.6 Hz, 2H), 7.37 − 7.10 (m, 3H), 6.96 (s, 1H), 4.45 − 4.24 (m, 1H), 4.24 − 3.92 (m, 1H), 3.45 − 3.10 (m, 1H), 2.84 (s, 3H), 2.09 − 1.90 (m, 1H), 1.88 − 1.47 (m, 5H), 1.40 − 0.70 (m, 5H) |

### Ex.132: Synthesis of (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-5-carboxylic acid

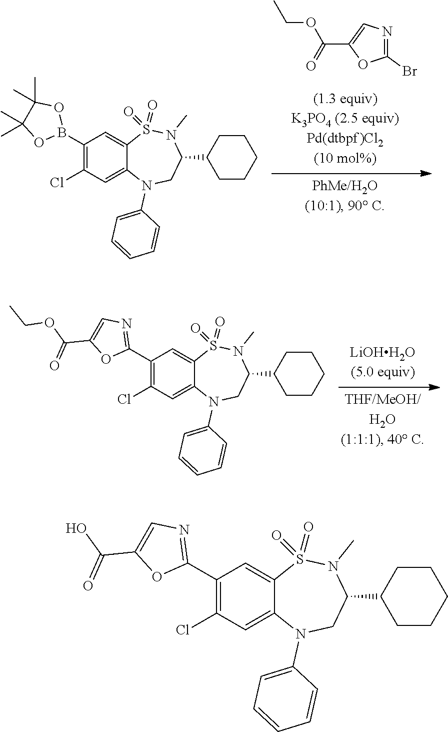 [SMILES: CCOC(=O)c1cnc(Br)o1 ; CN1[C@H](C2CCCCC2)CN(c2ccccc2)c2cc(Cl)c(B3OC(C)(C)C(C)(C)O3)cc2S1(=O)=O ; CCOC(=O)c1cnc(-c2cc3c(cc2Cl)N(c2ccccc2)C[C@@H](C2CCCCC2)N(C)S3(=O)=O)o1 ; CN1[C@H](C2CCCCC2)CN(c2ccccc2)c2cc(Cl)c(-c3ncc(C(=O)O)o3)cc2S1(=O)=O]

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (150.0 mg, 0.28 mmol, 1.0 eq) and ethyl 2-bromooxazole-5-carboxylate (80.9 mg, 0.37 mmol, 1.3 eq) in dioxane/H₂O (5.0/0.5 mL) was added K₃PO₄ (150.3 mg, 0.7 mmol, 2.5 eq) and Pd(dppf)C₁₂ (20.3 mg, 0.028 mmol, 0.1 eq) under N₂ atmosphere. The reaction mixture was then stirred at 90° C. overnight. The mixture was concentrated, diluted with water (50 mL) and extracted three times with EtOAc (50 mL each). The combined organic layers were concentrated to give a residue that was purified by silica gel chromatography (eluted with Petroleum ether: EtOAc=100:1) to give ethyl (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-5-carboxylate (85 mg, 55% yield) as a white solid. 1H NMR (300 MHz, CDCl₃): δ 8.58 (s, 1H), 7.87 (s, 1H), 7.48-7.39 (m, 2H), 7.25-7.15 (m, 3H), 6.87 (s, 1H), 4.60-4.38 (m, 3H), 4.25-4.17 (m, 1H), 3.15-3.05 (m, 1H), 2.98 (s, 3H), 2.30-2.16 (m, 1H), 1.90-1.84 (m, 1H), 1.81-1.60 (m, 4H), 1.41 (t, J=7.1 Hz, 3H), 1.29-1.23 (m, 4H), 1.13-1.05 (m, 1H).

Step 2

To a solution of ethyl (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-5-carboxylate (40.0 mg, 0.074 mmol, 1.0 eq) in THF/EtOH/H₂O (1.0/1.0/0.3 mL) was added LiOH (13.9 mg, 0.33 mmol, 4.5 eq). The reaction mixture was stirred at room temperature overnight and concentrated. The aqueous was acidified with 1 N HCl, and the resulting precipitate was collected via filtration, washed with water (5.0 mL), and dried to give (R)-2-(7-chloro-3-cyclohexyl-2-methyl-1,1-dioxido-5-phenyl-2,3,4,5-tetrahydrobenzo[f][1,2,5]thiadiazepin-8-yl)oxazole-5-carboxylic acid, Ex.132 (18 mg, 47% yield) as a white solid. ESI MS m/z=516.0 [M+H]⁺. ¹H NMR (400 MHz, DMSO-d6): δ 13.81 (s, 1H), 8.37 (s, 1H), 8.05 (s, 1H), 7.49-7.36 (m, 2H), 7.32-7.26 (m, 2H), 7.19 (t, J=7.1 Hz, 1H), 6.91 (s, 1H), 4.34-4.17 (m, 2H), 3.27-3.20 (m, 1H), 2.87 (s, 3H), 2.01-1.92 (m, 1H), 1.76-1.51 (m, 5H), 1.25-1.12 (m, 3H), 1.02-0.95 (m, 1H), 0.89-0.82 (m, 1H).

| Example | Structure | MS | NMR |
|---|---|---|---|
| 132 | 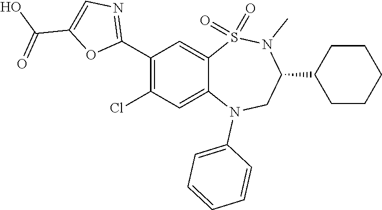 [SMILES: CN1[C@H](C2CCCCC2)CN(c2ccccc2)c2cc(Cl)c(-c3ncc(C(=O)O)o3)cc2S1(=O)=O] | [M + H]⁺ 516.0 | ¹H NMR (400 MHz, DMSO-d₆): δ 13.81 (s, 1H), 8.37 (s, 1H), 8.05 (s, 1H), 7.49 − 7.36 (m, 2H), 7.32 − 7.26 (m, 2H), 7.19 (t, J = 7.1 Hz, 1H), 6.91 (s, 1H), 4.34 − 4.17 (m, 2H), 3.27 − 3.20 (m, 1H), 2.87 (s, 3H), 2.01 − 1.92 (m, 1H), 1.76 − 1.51 (m, 5H), 1.25 − 1.12 (m, 3H), 1.02 − 0.95 (m, 1H), 0.89 − 0.82 (m, 1H) |

### Ex.133: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-1-methyl-1H-pyrazole-3-carboxylic acid

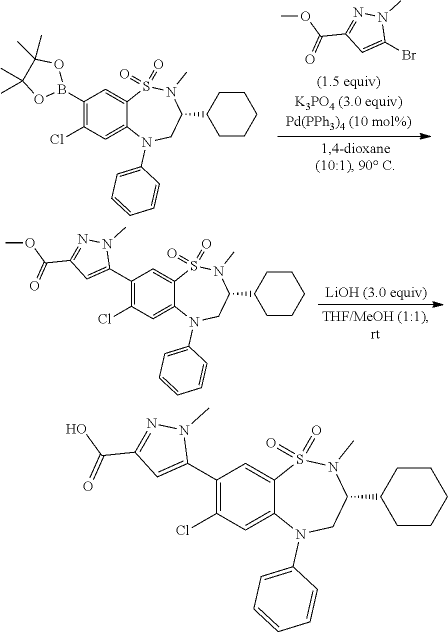

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (150 mg, 0.3 mmol, 1.0 eq) and methyl 5-bromo-1-methyl-1H-pyrazole-3-carboxylate (93 mg, 0.4 mmol, 1.5 eq) in dioxane (3.0 mL) and H₂O (0.3 mL) was added K₃PO₄ (180.2 mg, 0.8 mmol, 3.0 eq) and Pd(PPh₃)₄ (32.3 mg, 0.03 mmol, 0.1 eq) under N₂ atmosphere. The reaction mixture was heated at 90° C. overnight. The mixture was filtered, and the filtrate was diluted with EtOAc (5 mL). The reaction mixture was concentrated under reduced pressure to give a residue. The crude residue was purified by silica gel column chromatography (Petroleum ether/EtOAc=5:1) to give methyl (R)-5-(7-chloro-3-cyclohexyl-2-methyl-1,1-dioxido-5-phenyl-2,3,4,5-tetrahydrobenzo[f][1,2,5]thiadiazepin-8-yl)-1-methyl-1H-pyrazole-3-carboxylate (70 mg, 46% yield) as a white solid. ESI MS m/z=543.0 [M+H]⁺. ¹H NMR (400 MHz, CDCl₃): δ 7.83 (s, 1H), 7.42 (t, J=8.0 Hz, 2H), 7.23-7.10 (m, 3H), 6.96 (s, 1H), 6.85 (s, 1H), 4.43-4.18 (m, 2H), 3.94 (s, 3H), 3.82 (s, 3H), 3.21-3.08 (m, 1H), 2.97 (s, 3H), 2.22-2.13 (m, 1H), 1.89-1.63 (m, 5H), 1.35-1.24 (m, 3H), 0.97-0.86 (m, 2H).

Step 2

To a solution of methyl (R)-5-(7-chloro-3-cyclohexyl-2-methyl-1,1-dioxido-5-phenyl-2,3,4,5-tetrahydrobenzo[f][1,2,5]thiadiazepin-8-yl)-1-methyl-1H-pyrazole-3-carboxylate (75 mg, 0.14 mmol, 1.0 eq) in MeOH (1.0 mL) and THE (1.0 mL) was added LiOH (17.3 mg, 0.4 mmol 3.0 eq). The reaction mixture was stirred at rt overnight. The mixture was then concentrated, and the aqueous was adjusted to pH to 3-4 with 1 N HCl. The resulting precipitate was collected via filtration to give (R)-5-(7-chloro-3-cyclohexyl-2-methyl-1,1-dioxido-5-phenyl-2,3,4,5-tetrahydrobenzo[f][1,2,5]thiadiazepin-8-yl)-1-methyl-1H-pyrazole-3-carboxylic acid, Ex.133 (35 mg, yield 48% yield) as white solid. ESI MS m/z=529.2 [M+H]⁺. ¹H NMR (300 MHz, DMSO-d6): δ 7.80 (s, 1H), 7.37 (t, J=8.1 Hz, 2H), 7.38-6.98 (m, 4H), 6.81 (s, 1H), 4.45-4.24 (m, 1H), 4.03-3.78 (m, 1H), 3.74 (s, 3H), 3.55-3.10 (m, 1H), 2.76 (s, 3H), 2.03-1.80 (m, 2H), 1.78-1.52 (m, 4H), 1.32-1.07 (m, 3H), 1.07-0.80 (m, 2H).

| Example | Structure | MS | NMR |
|---|---|---|---|
| 133 | 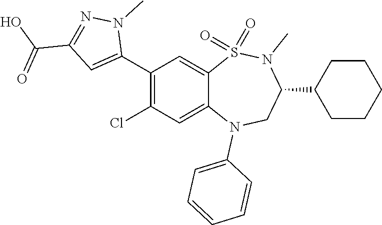 [SMILES: CN1[C@H](C2CCCCC2)CN(c2ccccc2)c2cc(Cl)c(-c3cc(C(=O)O)nn3C)cc2S1(=O)=O] | [M + H]⁺ 529.2 | ¹H NMR (300 MHz, DMSO-d₆): δ 7.80 (s, 1H), 7.37 (t, J = 8.1 Hz, 2H), 7.38 − 6.98 (m, 4H), 6.81 (s, 1H), 4.45 − 4.24 (m, 1H), 4.03 − 3.78 (m, 1H), 3.74 (s, 3H), 3.55 − 3.10 (m, 1H), 2.76 (s, 3H), 2.03 − 1.80 (m, 2H), 1.78 − 1.52 (m, 4H), 1.32 − 1.07 (m, 3H), 1.07 − 0.80 (m, 2H). |

The following compounds were prepared using a procedure analogous to that used for Ex.133:

| Example | Structure | MS | NMR |
|---|---|---|---|
| 134 | 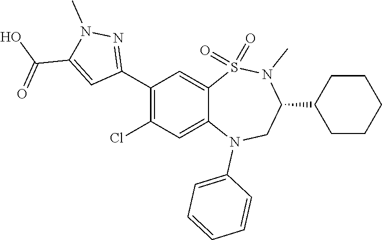 [SMILES: CN1[C@H](C2CCCCC2)CN(c2ccccc2)c2cc(Cl)c(-c3cc(C(=O)O)n(C)n3)cc2S1(=O)=O] | [M − H]⁻ 526.9 | ¹H NMR (400 MHz, DMSO-d₆): δ 13.57 (brs, 1H), 8.24 (s, 1H), 7.60 − 7.28 (m, 3H), 7.20 (s, 1H), 7.12 − 6.80 (m, 3H), 4.50 − 4.25 (m, 1H), 4.18 (s, 3H), 3.74 (s, 1H), 3.52 − 3.10 (m, 1H), 2.69 (s, 3H), 2.07 − 1.82 (m, 2H), 1.80 − 1.48 (m, 4H), 1.40 − 1.07 (m, 3H), 1.07 − 0.83 (m, 2H) |

### Ex. 135: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-1H-pyrazole-5-carboxylic acid

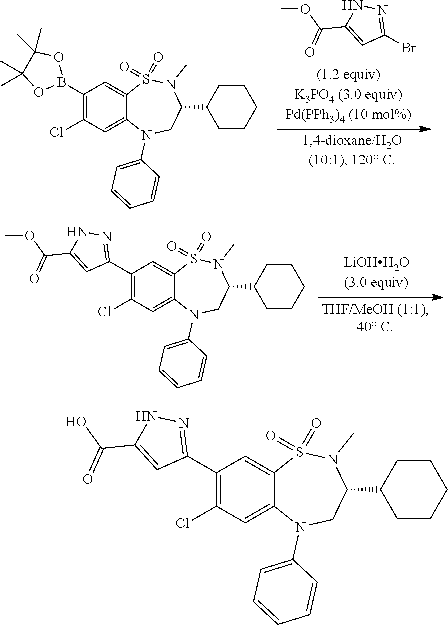

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100 mg, 0.2 mmol, 1.0 eq) and methyl 3-bromo-1H-pyrazole-5-carboxylate (46.3 mg, 0.24 mmol, 1.2 eq) in 1,4-dioxane/H₂O (1.0 mL/0.1 mL) was added K₃PO₄ (119.7 mg, 0.6 mmol, 3.0 eq) and Pd(dppf)Cl₂ (13.8 mg, 0.02 mmol, 0.1 eq) under N₂ atmosphere. The reaction mixture was then stirred at 120° C. for 2 h. The mixture was filtered, and the filtrate was concentrated to give a residue. The crude material was purified by silica gel column chromatography (Petroleum ether/EtOAc=50:1 to 10:1 to 3:1) to provide methyl (R)-3-(7-chloro-3-cyclohexyl-2-methyl-1,1-dioxido-5-phenyl-2,3,4,5-tetrahydrobenzo[f][1,2,5]thiadiazepin-8-yl)-1H-pyrazole-5-carboxylate (30 mg, 30% yield) as a white solid. ESI MS m/z=529.0 [M+H]⁺. ¹H NMR (300 MHz, DMSO-d6): δ 7.77-7.63 (m, 1H), 7.43-6.90 (m, 8H), 4.42-4.27 (m, 1H), 3.92 (s, 3H), 3.40-3.20 (m, 2H), 2.82-2.63 (m, 3H), 2.05-1.82 (m, 2H), 1.78-1.50 (m, 4H), 1.32-1.22 (m, 5H) ppm.

Step 2

To a solution of methyl (R)-3-(7-chloro-3-cyclohexyl-2-methyl-1,1-dioxido-5-phenyl-2,3,4,5-tetrahydrobenzo[f][1,2,5]thiadiazepin-8-yl)-1H-pyrazole-5-carboxylate (40 mg, 0.1 mmol, 1.0 eq) in THF/MeOH (0.5 mL/0.5 mL) was added LiOH—H₂O (9.6 mg, 0.2 mmol, 3.0 eq). The reaction mixture was then stirred at 40° C. overnight. The reaction mixture was concentrated under reduced pressure to give a crude product. The crude residue was adjusted to pH 4 with HCl (1 N), and the aqueous phase was extracted twice with EtOAc (20 mL each). The combined organic layers were concentrated and the residue was purified by RPHPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)-1H-pyrazole-5-carboxylic acid, Ex.135 (3 mg, 8% yield) as a white solid. ESI MS m/z=515.0. ¹H NMR (400 MHz, DMSO-d₆): δ 13.81 (brs, 1H), 8.29 (s, 1H), 7.50-6.80 (m, 8H), 4.50-4.22 (m, 1H), 3.95-3.55 (m, 2H), 2.67 (s, 3H), 2.10-1.82 (m, 2H), 1.80-1.50 (m, 4H), 1.35-0.70 (m, 5H).

| Example | Structure | MS | NMR |
|---|---|---|---|
| 135 | 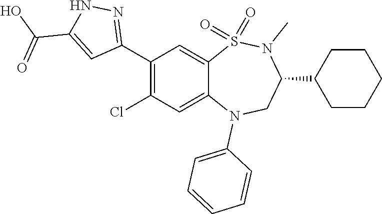 [SMILES: CN1[C@H](C2CCCCC2)CN(c2ccccc2)c2cc(Cl)c(-c3cc(C(=O)O)[nH]n3)cc2S1(=O)=O] | [M + H]⁺ 515.0 | ¹H NMR (400 MHz, DMSO-d₆): δ 13.81 (brs, 1H), 8.29 (s, 1H), 7.50 − 6.80 (m, 8H), 4.50 − 4.22 (m, 1H), 3.95 − 3.55 (m, 2H), 2.67 (s, 3H), 2.10 − 1.82 (m, 2H), 1.80 − 1.50 (m, 4H), 1.35 − 0.70 (m, 5H) |

### Ex.136: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)nicotinic acid

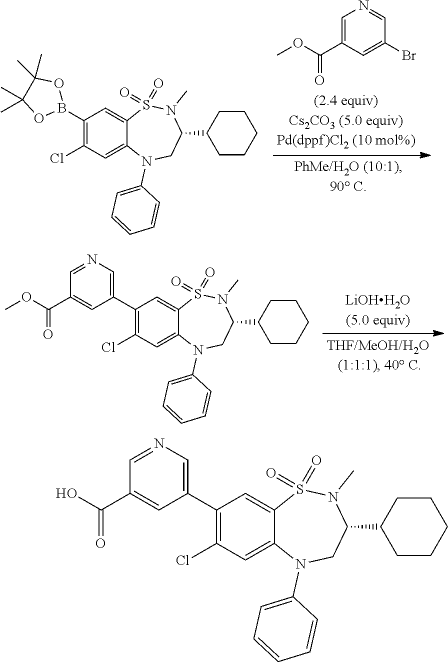

Step 1

A mixture of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.19 mmol, 1.0 eq), methyl 5-bromonicotinate (100.0 mg, 0.46 mmol, 2.4 equiv, CAS #29681-44-5), Pd(dppf)Cl₂ (17.0 mg, 0.02 mmol, 0.1 eq) and Cs₂CO₃ (300.0 mg, 0.92 mmol, 5.0 eq) in toluene (5.0 mL) and H₂O (0.5 mL) were stirred for 2 h at 90° C. under a nitrogen atmosphere. The reaction was quenched by the addition of H₂O (10 mL) at room temperature and extracted with EtOAc (30 mL×3). The combined organic layers were dried over anhydrous Na₂SO₄ and concentrated under reduced pressure. The residue was purified by silica gel column chromatography (eluting with EtOAc/Petroleum ether=20/80) to give methyl (R)-5-(7-chloro-3-cyclohexyl-2-methyl-1,1-dioxido-5-phenyl-2,3,4,5-tetrahydrobenzo[f][1,2,5]thiadiazepin-8-yl)nicotinate (60.0 mg, 59.0%) as a white solid. ESI MS m/z=540.1 [M+H]⁺.

Step 2

A mixture of (R)-5-(7-chloro-3-cyclohexyl-2-methyl-1,1-dioxido-5-phenyl-2,3,4,5-tetrahydrobenzo[f][1,2,5]thiadiazepin-8-yl)nicotinate (60.0 mg, 0.1 mmol, 1.0 equiv) and LiOH·H₂O (20.0 mg, 0.5 mmol, 5.0 eq.) in THE (1.0 mL), MeOH (1.0 mL) and H₂O (1.0 mL) was stirred for 2 h at 40° C. The resulting mixture was concentrated under vacuum, and the aqueous layer was adjusted to pH 6 with HCl (1N). The resulting mixture was extracted three times with EtOAc (20 mL each). The combined organic layers were dried over anhydrous Na₂SO₄ and concentrated under reduced pressure. The residue was purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)nicotinic acid, Ex.136 (20.8 mg, 36% yield) as a white solid. ESI MS m/z=526.0 [M+H]⁺. ¹H NMR (300 MHz, DMSO-d₆): δ 8.14-8.05 (m, 2H), 8.03-7.92 (m, 2H), 7.40-7.31 (m, 2H), 7.38-6.92 (m, 4H), 4.50-4.30 (m, 1H), 3.92-3.82 (m, 1H), 3.29-3.20 (m, 1H), 2.76 (s, 3H), 2.01-1.81 (m, 2H), 1.80-1.55 (m, 4H), 1.26-1.12 (m, 3H), 1.06-0.09 (m, 2H).

| Example | Structure | MS | NMR |
|---|---|---|---|
| 136 | 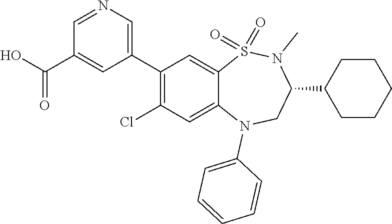 [SMILES: CN1[C@H](C2CCCCC2)CN(c2ccccc2)c2cc(Cl)c(-c3cncc(C(=O)O)c3)cc2S1(=O)=O] | [M + H]⁺ 526.0 | ¹H NMR (300 MHz, DMSO- d₆): δ 8.14 − 8.05 (m, 2H), 8.03 − 7.92 (m, 2H), 7.40 − 7.31 (m, 2H), 7.38 − 6.92 (m, 4H), 4.50 − 4.30 (m, 1H), 3.92 − 3.82 (m, 1H), 3.29 − 3.20 (m, 1H), 2.76 (s, 3H), 2.01-1.81 (m, 2H), 1.80 − 1.55 (m, 4H), 1.26 − 1.12 (m, 3H), 1.06 − 0.09 (m, 2H) |

### Ex.137: Synthesis of (R)-4-(7-chloro-3-cyclohexyl-2-methyl-1,1-dioxido-5-phenyl-2,3,4,5-tetrahydrobenzo[f][1,2,5]thiadiazepin-8-yl)picolinic acid

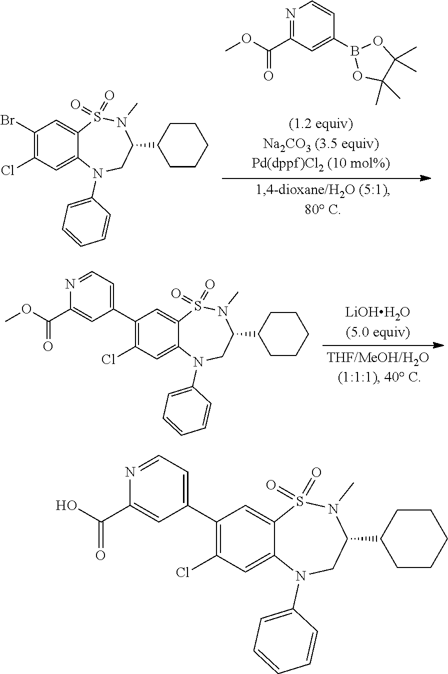

Step 1

A mixture of (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 equiv), methyl 4-(4,4,5,5-tetramethyl-1,3,2-dioxaborolan-2-yl)picolinate (65 mg, 0.25 mmol, 1.2 equiv, CAS #957062-72-5), Na₂CO₃ (79 mg, 0.74 mmol, 3.5 eq) and Pd(dppf)Cl₂ (15 mg, 0.02 mmol, 10 mol %) in 1,4-dioxane (5 mL) and H₂O (1 mL) was stirred for 2 h at 80° C. under a nitrogen atmosphere. Upon cooling to room temperature, the reaction was quenched by the addition of H₂O (50 mL) at room temperature. The resulting mixture was extracted three times with EtOAc (50 mL each). The combined organic layers were dried over anhydrous Na₂SO₄, filtered, and concentrated under reduced pressure. Purification of the residue by silica gel column chromatography (eluting with EtOAc/Petroleum ether=20/80) afforded methyl (R)-4-(7-chloro-3-cyclohexyl-2-methyl-1,1-dioxido-5-phenyl-2,3,4,5-tetrahydrobenzo[f][1,2,5]thiadiazepin-8-yl)picolinate (40 mg, 36% yield) as a white solid. ESI MS m/z=540.0 [M+H]⁺.

Step 2

A mixture of methyl (R)-4-(7-chloro-3-cyclohexyl-2-methyl-1,1-dioxido-5-phenyl-2,3,4,5-tetrahydrobenzo[f][1,2,5]thiadiazepin-8-yl)picolinate (40 mg, 0.07 mmol, 1.0 eq.) and LiOH—H₂O (16 20 mg, 0.37 mmol, 5.0 eq.) in THF (1 mL), MeOH (1 mL) and H₂O (1 mL) was stirred for 3 h at 40° C. The resulting mixture was extracted three times with EtOAc (20 mL each). The combined organic layers were dried over anhydrous Na₂SO₄. The residue was then purified by RPHPLC to afford (R)-4-(7-chloro-3-cyclohexyl-2-methyl-1,1-dioxido-5-phenyl-2,3,4,5-tetrahydrobenzo[f][1,2,5]thiadiazepin-8-yl)picolinic acid, Ex.137 (11.3 mg, 29% yield) as a yellow solid. ESI MS m/z=526.0 [M+H]⁺. ¹H NMR (300 MHz, DMSO-d₆): δ 8.90-8.72 (m, 1H), 8.13 (s, 1H), 7.85-7.76 (m, 2H), 7.42-7.30 (m, 2H), 7.26-6.98 (m, 4H), 4.46-4.28 (m, 1H), 3.92-3.65 (m, 2H), 2.75 (s, 3H), 2.06-1.82 (m, 2H), 1.74-1.56 (m, 4H), 1.25-1.12 (m, 3H), 1.06-0.86 (m, 2H).

| Example | Structure | MS | NMR |
|---|---|---|---|
| 137 | 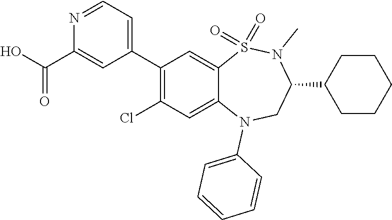 [SMILES: CN1[C@H](C2CCCCC2)CN(c2ccccc2)c2cc(Cl)c(-c3ccnc(C(=O)O)c3)cc2S1(=O)=O] | [M + H]⁺ 526.0 | ¹H NMR (300 MHz, DMSO-d₆): δ 8.90 − 8.72(m, 1H), 8.13 (s, 1H), 7.85 − 7.76 (m, 2H), 7.42 − 7.30 (m, 2H), 7.26− 6.98 (m, 4H), 4.46 − 4.28 (m, 1H), 3.92 − 3.65 (m, 2H), 2.75 (s, 3H), 2.06 − 1.82 (m, 2H), 1.74 − 1.56 (m, 4H), 1.25 − 1.12 (m, 3H), 1.06 − 0.86 (m, 2H). |

### Ex.138: Synthesis of (R)-6-(7-chloro-3-cyclohexyl-2-methyl-1,1-dioxido-5-phenyl-2,3,4,5-tetrahydrobenzo[f][1,2,5]thiadiazepin-8-yl)picolinic acid

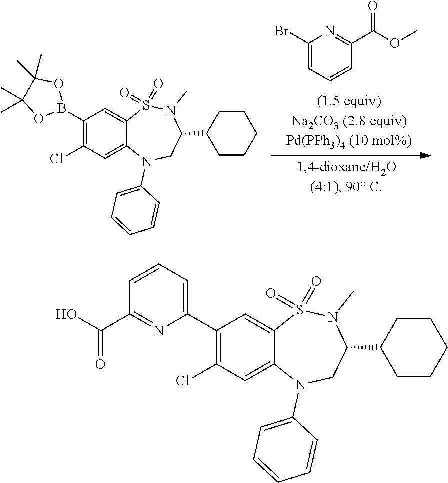 [SMILES: CN1[C@H](C2CCCCC2)CN(c2ccccc2)c2cc(Cl)c(B3OC(C)(C)C(C)(C)O3)cc2S1(=O)=O ; COC(=O)c1cccc(Br)n1 ; CN1[C@H](C2CCCCC2)CN(c2ccccc2)c2cc(Cl)c(-c3cccc(C(=O)O)n3)cc2S1(=O)=O]

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (200 mg, 0.4 mmol, 1.0 eq) and methyl 6-bromopicolinate (114 mg, 0.6 mmol, 1.5 eq) in 1,4-dioxane (4 mL) and H₂O (1 mL) was added Na₂CO₃ (120 mg, 1.1 mmol, 2.8 eq) and Pd(PPh₃)₄ (44 mg, 0.04 mmol, 0.1 eq) under N₂ atmosphere. The reaction mixture was heated at 90° C. for 2 h. The mixture was filtered, and the filtrate was concentrated in vacuo. The residue was purified by RPHPLC to afford (R)-6-(7-chloro-3-cyclohexyl-2-methyl-1,1-dioxido-5-phenyl-2,3,4,5-tetrahydrobenzo[f][1,2,5]thiadiazepin-8-yl)picolinic acid, Ex.138 (20.0 mg, yield 10% yield) as a white solid. ESI MS m/z=526.3 [M+H]⁺. ¹H NMR (400 MHz, DMSO-d₆): δ 13.41 (brs, 1H), 8.34-7.88 (m, 4H), 7.67-6.76 (m, 6H), 4.38 (d, J=14.8 Hz, 1H), 3.85 (s, 1H), 3.30-3.20 (m, 1H), 2.76 (s, 3H), 2.01-1.82 (m, 2H), 1.80-1.40 (m, 4H), 1.35-1.04 (m, 3H), 1.04-0.70 (m, 2H).

| Example | Structure | MS | NMR |
|---|---|---|---|
| 138 | 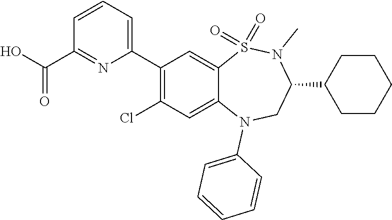 [SMILES: CN1[C@H](C2CCCCC2)CN(c2ccccc2)c2cc(Cl)c(-c3cccc(C(=O)O)n3)cc2S1(=O)=O] | [M + H]⁺ 526.3 | ¹H NMR (400 MHz, DMSO-d₆): δ 13.41 (brs, 1H), 8.34 − 7.88 (m, 4H), 7.67 − 6.76 (m, 6H), 4.38 (d, J = 14.8 Hz, 1H), 3.85 (s, 1H), 3.30 − 3.20 (m, 1H), 2.76 (s, 3H), 2.01 − 1.82 (m, 2H), 1.80 − 1.40 (m, 4H), 1.35 − 1.04 (m, 3H), 1.04 − 0.70 (m, 2H) |

### Ex.139: Synthesis of (R)-2-(7-chloro-3-cyclohexyl-2-methyl-1,1-dioxido-5-phenyl-2,3,4,5-tetrahydrobenzo[f][1,2,5]thiadiazepin-8-yl)isonicotinic acid

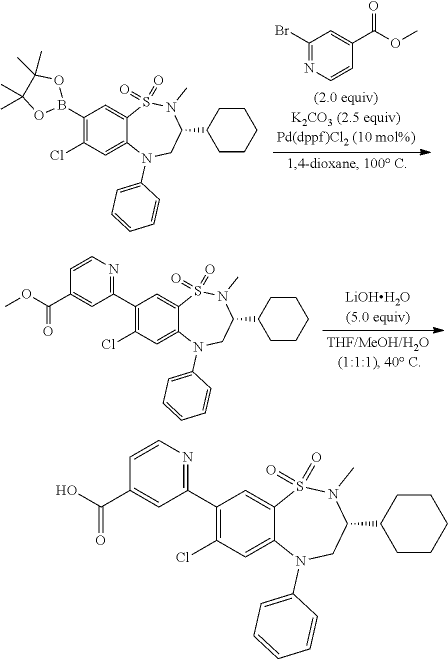

Step 1

A mixture of methyl 2-bromoisonicotinate (81.0 mg, 0.37 mmol, 2.0 eq), (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.19 mmol, 1.0 eq), K₂CO₃ (65.0 mg, 0.47 mmol, 2.5 eq) and Pd(dppf)C₁₂ (15.0 mg, 0.02 mmol, 0.1 eq) in 1,4-dioxane (4.0 mL) was stirred for 2 h at 100° C. under a nitrogen atmosphere. The resulting mixture was cooled to room temperature. The reaction was quenched by the addition of H₂O (15 mL) at room temperature. The resulting mixture was extracted with EtOAc (15 mL×3). The combined organic layers were dried over anhydrous Na₂SO₄, filtered, and concentrated under reduced pressure. The residue was purified by silica gel column chromatography (eluting with EtOAc/Petroleum ether=1/10) to give methyl (R)-2-(7-chloro-3-cyclohexyl-2-methyl-1,1-dioxido-5-phenyl-2,3,4,5-tetrahydrobenzo[f][1,2,5]thiadiazepin-8-yl)isonicotinate (50.0 mg, 49% yield) as a yellow solid.

Step 2

A mixture of methyl (R)-2-(7-chloro-3-cyclohexyl-2-methyl-1,1-dioxido-5-phenyl-2,3,4,5-tetrahydrobenzo[f][1,2,5]thiadiazepin-8-yl)isonicotinate (40.0 mg, 0.07 mmol, 1.0 eq) and lithium hydroxide monohydrate (16.0 mg, 0.37 mmol, 5.0 eq) in MeOH (1 mL), H₂O (1 mL) and THF (1 mL) was stirred for 2 h at 40° C. The resulting mixture was concentrated under vacuum. The residue was adjusted to pH 6 with HCl (1 N), and the resulting mixture was extracted three times with EtOAc (15 mL each). The combined organic layers were dried over anhydrous Na₂SO₄, filtered, and concentrated under reduced pressure. The resulting residue was purified by RPHPLC to afford (R)-2-(7-chloro-3-cyclohexyl-2-methyl-1,1-dioxido-5-phenyl-2,3,4,5-tetrahydrobenzo[f][1,2,5]thiadiazepin-8-yl)isonicotinic acid, Ex.139 (3.8 mg, 10% yield) as a yellow solid. ESI MS m/z=526.0 [M+H]⁺. ¹H NMR (300 MHz, DMSO-d₆): δ 8.90 (d, J=5.1 Hz, 1H), 8.18 (s, 1H), 8.06 (s, 1H), 7.89-7.80 (m, 1H), 7.36 (t, J=7.8 Hz, 2H), 7.28-6.99 (m, 4H), 4.52-4.28 (m, 1H), 3.99-3.85 (m, 1H), 3.50-3.40 (m, 1H), 2.75 (s, 3H), 2.01-1.89 (m, 2H), 1.81-1.49 (m, 4H), 1.29-0.90 (m, 5H).

| Example | Structure | MS | NMR |
|---|---|---|---|
| 139 | 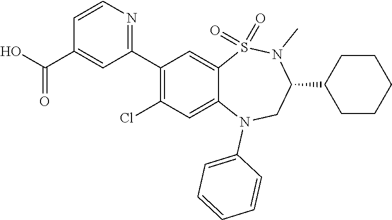 [SMILES: CN1[C@H](C2CCCCC2)CN(c2ccccc2)c2cc(Cl)c(-c3cc(C(=O)O)ccn3)cc2S1(=O)=O] | [M + H]⁺ 526.0 | ¹H NMR (300 MHz, DMSO-d₆): δ 8.90 (d, J = 5.1 Hz, 1H), 8.18 (s, 1H), 8.06 (s, 1H), 7.89 − 7.80 (m, 1H), 7.36 (t, J = 7.8 Hz, 2H), 7.28 − 6.99 (m, 4H), 4.52 − 4.28 (m, 1H), 3.99 − 3.85 (m, 1H), 3.50 − 3.40 (m, 1H), 2.75 (s, 3H), 2.01 − 1.89 (m, 2H), 1.81 − 1.49 (m, 4H), 1.29 − 0.90 (m, 5H) |

### Ex.140: Synthesis of (R)-6-(7-chloro-3-cyclohexyl-2-methyl-1,1-dioxido-5-phenyl-2,3,4,5-tetrahydrobenzo[f][1,2,5]thiadiazepin-8-yl)pyrimidine-4-carboxylic acid

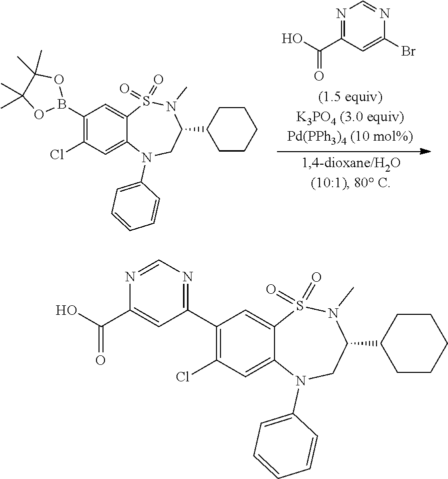 [SMILES: CN1[C@H](C2CCCCC2)CN(c2ccccc2)c2cc(Cl)c(B3OC(C)(C)C(C)(C)O3)cc2S1(=O)=O ; O=C(O)c1cc(Br)ncn1 ; CN1[C@H](C2CCCCC2)CN(c2ccccc2)c2cc(Cl)c(-c3cc(C(=O)O)ncn3)cc2S1(=O)=O]

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100.0 mg, 0.2 mmol, 1.0 eq) and 6-bromopyrimidine-4-carboxylic acid (44.7 mg, 0.3 mmol, 1.5 eq) in 1,4-dioxane (3.0 mL) and H₂O (0.3 mL) was added Pd(PPh₃)₄ (21.9 mg, 0.02 mmol, 0.1 eq) and K₃PO₄ (119.7 mg, 0.6 mmol, 3.0 eq) under N₂. The reaction mixture was heated at 80° C. overnight. The mixture was filtered, the filtrate was diluted with EtOAc (10 mL) and washed with H₂O (5 mL). The mixture was adjusted to pH 3 to 4 with 1 N HCl, and extracted three times with EtOAc (5 mL each). The organic phase was concentrated and the residue was purified RPHPLC to afford (R)-6-(7-chloro-3-cyclohexyl-2-methyl-1,1-dioxido-5-phenyl-2,3,4,5-tetrahydrobenzo[f][1,2,5]thiadiazepin-8-yl)pyrimidine-4-carboxylic acid, Ex.140 (25.0 mg, 25% yield) as a white solid. ESI MS m/z=527.0 [M+H]⁺. ¹H NMR (400 MHz, DMSO-d₆): δ 13.98 (brs, 1H), 9.47 (d, J=1.2 Hz, 1H), 8.37 (d, J=1.2 Hz, 1H), 8.19 (s, 1H), 7.41 (t, J=7.6 Hz, 2H), 7.32-6.95 (m, 4H), 4.47-4.24 (m, 1H), 4.01 (s, 1H), 3.32-3.20 (m, 1H), 2.82 (s, 3H), 2.05-1.50 (m, 6H), 1.30-1.05 (m, 3H), 1.05-0.80 (m, 2H).

| Example | Structure | MS | NMR |
|---|---|---|---|
| 140 | 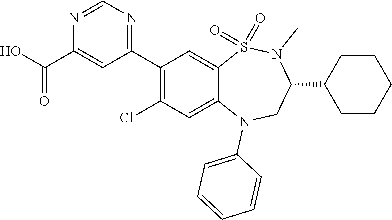 [SMILES: CN1[C@H](C2CCCCC2)CN(c2ccccc2)c2cc(Cl)c(-c3cc(C(=O)O)ncn3)cc2S1(=O)=O] | [M + H]⁺ 527.0 | ¹H NMR (400 MHz, DMSO-d6): δ 13.98 (brs, 1H), 9.47 (d, J = 1.2 Hz, 1H), 8.37 (d, J = 1.2 Hz, 1H), 8.19 (s, 1H), 7.41 (t, J = 7.6 Hz, 2H), 7.32 − 6.95 (m, 4H), 4.47 − 4.24 (m, 1H), 4.01 (s, 1H), 3.32 − 3.20 (m, 1H), 2.82 (s, 3H), 2.05 − 1.50 (m, 6H), 1.30 − 1.05 (m, 3H), 1.05 − 0.80 (m, 2H). |

### Ex.141: Synthesis of (R)-6-(7-chloro-3-cyclohexyl-2-methyl-1,1-dioxido-5-phenyl-2,3,4,5-tetrahydrobenzo[f][1,2,5]thiadiazepin-8-yl)-3-fluoropicolinic acid

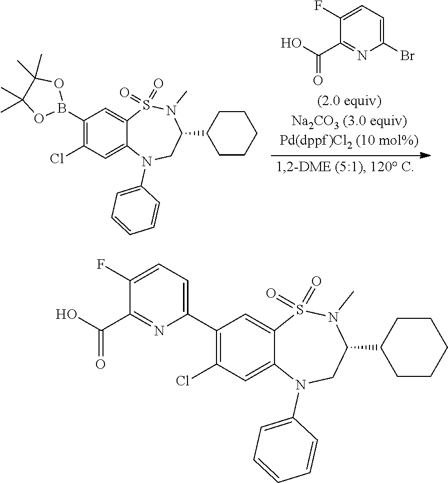 [SMILES: CN1[C@H](C2CCCCC2)CN(c2ccccc2)c2cc(Cl)c(B3OC(C)(C)C(C)(C)O3)cc2S1(=O)=O ; O=C(O)c1nc(Br)ccc1F ; CN1[C@H](C2CCCCC2)CN(c2ccccc2)c2cc(Cl)c(-c3ccc(F)c(C(=O)O)n3)cc2S1(=O)=O]

Step 1

A mixture of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100 mg, 0.19 mmol, 1.0 eq), 6-bromo-3-fluoropicolinic acid (84 mg, 0.38 mmol, 2.0 eq), Na₂CO₃ (60 mg, 0.57 mmol, 3.0 eq) and Pd(dppf)C₁₂ (14 mg, 0.02 mmol, 0.1 eq) in DME (3 mL) and H₂O (0.6 mL) was stirred for 30 min at 120° C. under microwave irradiation. The resulting mixture was cooled to room temperature. The reaction was quenched by the addition of H₂O (20 mL) at room temperature. The resulting mixture was extracted three times with EtOAc (20 mL each). The combined organic layers were dried over anhydrous Na₂SO₄ and concentrated under reduced pressure. The residue was purified by RPHPLC to afford (R)-6-(7-chloro-3-cyclohexyl-2-methyl-1,1-dioxido-5-phenyl-2,3,4,5-tetrahydrobenzo[f][1,2,5]thiadiazepin-8-yl)-3-fluoropicolinic acid, Ex.141 (16.7 mg, 16% yield) as a white solid. ESI MS m/z=544.0 [M+H]⁺. ¹H NMR (400 MHz, DMSO-d₆): δ 13.77 (brs, 1H), 8.10-7.90 (m, 3H), 7.36 (t, J=7.6 Hz, 2H), 7.28-6.80 (m, 4H), 4.37 (d, J=14.8 Hz, 1H), 3.85 (s, 1H), 3.50-3.10 (m, 1H), 2.76 (s, 3H), 2.05-1.82 (m, 2H), 1.80-1.48 (m, 4H), 1.38-1.06 (m, 3H), 1.05-0.75 (m, 2H).

| Example | Structure | MS | NMR |
|---|---|---|---|
| 141 | 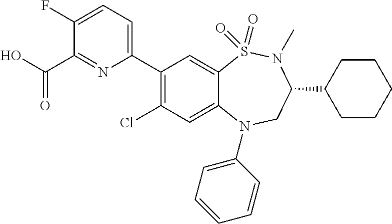 [SMILES: CN1[C@H](C2CCCCC2)CN(c2ccccc2)c2cc(Cl)c(-c3ccc(F)c(C(=O)O)n3)cc2S1(=O)=O] | [M + H]⁺ 544.0 | ¹H NMR (400 MHz, DMSO-d₆): δ 13.77 (brs, 1H), 8.10-7.90 (m, 3H), 7.36 (t, J = 7.6 Hz, 2H), 7.28-6.80 (m, 4H), 4.37 (d, J = 14.8 Hz, 1H), 3.85 (s, 1H), 3.50-3.10 (m, 1H), 2.76 (s, 3H), 2.05-1.82 (m, 2H), 1.80-1.48 (m, 4H), 1.38-1.06 (m, 3H), 1.05-0.75 (m, 2H) |

### Ex.142: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-5-(trifluoromethyl)benzoic acid

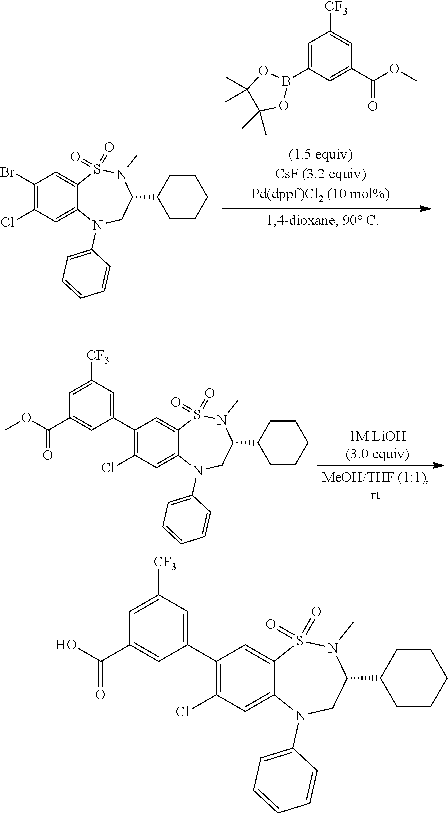

Step 1

To a solution of (R)-8-bromo-7-chloro-3-cyclohexyl-2-methyl-5-phenyl-2,3,4,5-tetrahydrobenzo[f][1,2,5]thiadiazepine 1,1-dioxide (300 mg, 0.6 mmol, 1.0 eq) and methyl 3-(4,4,5,5-tetramethyl-1,3,2-dioxaborolan-2-yl)-5-(trifluoromethyl)benzoate (307 mg, 0.9 mmol, 1.5 eq) in 1,4-dioxane (10 mL) was added CsF (282 mg, 1.9 mmol, 3.2 eq) and Pd(dppf)C₁₂ (45 mg, 0.06 mmol, 0.1 eq) under N₂ atmosphere. The reaction mixture was heated at 90° C. overnight. Upon cooling to room temperature, the reaction mixture was filtered through a pad of celite and concentrated. The crude product was purified by silica gel column chromatography (Petroleum ether/EtOAc=5:1 to 3:1) to give methyl (R)-3-(7-chloro-3-cyclohexyl-2-methyl-1,1-dioxido-5-phenyl-2,3,4,5-tetrahydrobenzo[f][1,2,5]thiadiazepin-8-yl)-5-(trifluoromethyl)benzoate (110 mg, 29% yield) as a white solid. ESI MS m/z=606.9 [M+H]⁺.

Step 2

To a solution of methyl (R)-3-(7-chloro-3-cyclohexyl-2-methyl-1,1-dioxido-5-phenyl-2,3,4,5-tetrahydrobenzo[f][1,2,5]thiadiazepin-8-yl)-5-(trifluoromethyl)benzoate (110 mg, 0.2 mmol, 1.0 eq) in MeOH (2 mL) and THE (2 mL) was added lithium hydroxide (13 mg, 0.5 mmol, 1 N, 3.0 eq). The reaction mixture was stirred at room temperature overnight. The mixture was adjusted to pH to 2-3 with 1N HCl and the resulting precipitate was collected via filtration to give (R)-3-(7-chloro-3-cyclohexyl-2-methyl-1,1-dioxido-dioxido-5-phenyl-2,3,4,5-tetrahydrobenzo[f][1,2,5]thiadiazepin-8-yl)-5-(trifluoromethyl)benzoic acid, Ex.142 (40 mg, 37.2%) as a white solid. ESI MS m/z=593.0 [M+H]⁺. ¹H NMR (300 MHz, DMSO-d₆): δ 8.23 (d, J=5.1 Hz, 2H), 8.00 (s, 1H), 7.81 (s, 1H), 7.45-7.18 (m, 3H), 7.18-6.88 (m, 3H), 4.40-4.34 (m, 1H), 3.72 (s, 1H), 3.30-3.22 (m, 1H), 2.71 (s, 3H), 2.10-1.83 (m, 2H), 1.80-1.50 (m, 4H), 1.40-0.77 (m, 5H).

| Example | Structure | MS | NMR |
|---|---|---|---|
| 142 | 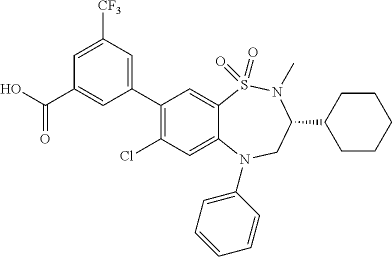 [SMILES: CN1[C@H](C2CCCCC2)CN(c2ccccc2)c2cc(Cl)c(-c3cc(C(=O)O)cc(C(F)(F)F)c3)cc2S1(=O)=O] | [M + H]⁺ 593.0 | ¹H NMR (300 MHz, DMSO- d₆): δ 8.90 (d, J = 5.1 Hz, 1H), 8.18 (s, 1H), 8.06 (s, 1H), 7.89- 7.80 (m, 1H), 7.36 (t, J = 7.8 Hz, 2H), 7.28-6.99 (m, 4H), 4.52- 4.28 (m, 1H), 3.99-3.85 (m, 1H), 3.50-3.40 (m, 1H), 2.75 (s, 3H), 2.01-1.89 (m, 2H), 1.81-1.49 (m, 4H), 1.29-0.90 (m, 5H) |

### Ex.143: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-2-(trifluoromethyl)benzoic acid

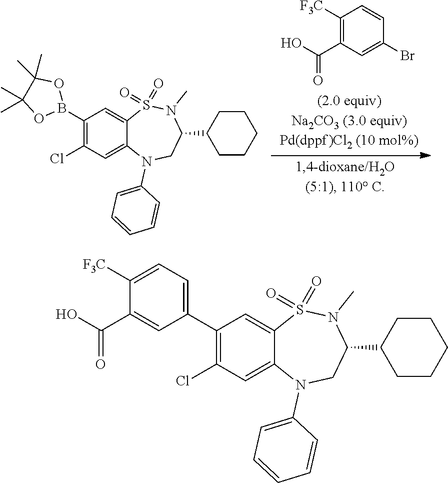 [SMILES: CN1[C@H](C2CCCCC2)CN(c2ccccc2)c2cc(Cl)c(B3OC(C)(C)C(C)(C)O3)cc2S1(=O)=O ; O=C(O)c1cc(Br)ccc1C(F)(F)F ; CN1[C@H](C2CCCCC2)CN(c2ccccc2)c2cc(Cl)c(-c3ccc(C(F)(F)F)c(C(=O)O)c3)cc2S1(=O)=O]

Step 1

A mixture of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (50.0 mg, 0.09 mmol, 1.0 eq.), 5-bromo-2-(trifluoromethyl)benzoic acid (51.0 mg, 0.19 mmol, 2.0 eq.), Na₂CO₃ (30.0 mg, 0.28 mmol, 3.0 eq.) and Pd(dppf)C₁₂ (7.0 mg, 0.01 mmol, 0.1 eq.) in 1,4-dioxane (2.0 mL) and H₂O (0.4 mL) was stirred for 2 h at 110° C. under a nitrogen atmosphere. The resulting mixture was cooled to room temperature. The reaction was quenched by the addition of H₂O (15 mL) at room temperature. The resulting mixture was extracted three times with EtOAc (15 mL each). The combined organic layers were dried over anhydrous Na₂SO₄, filtered, and concentrated under reduced pressure. The residue was purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-(trifluoromethyl)benzoic acid, Ex.143 (15.0 mg, 27%) as a white solid. ESI MS m/z=593.0 [M+H]⁺. ¹H NMR (300 MHz, DMSO-d₆): δ 13.77 (brs, 1H), 7.91-7.76 (m, 4H), 7.37-7.23 (m, 3H), 7.07-6.99 (m, 3H), 4.50-4.28 (m, 1H), 3.80-3.72 (m, 1H), 3.47-3.32 (m, 1H), 2.72 (s, 3H), 1.97-1.86 (m, 2H), 1.75-1.58 (m, 4H), 1.27-1.13 (m, 3H), 1.04-0.90 (m, 2H).

| Example | Structure | MS |
|---|---|---|
| 143 | 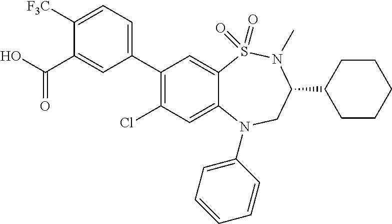 [SMILES: CN1[C@H](C2CCCCC2)CN(c2ccccc2)c2cc(Cl)c(-c3ccc(C(F)(F)F)c(C(=O)O)c3)cc2S1(=O)=O] | [M + H]⁺ 593.0 |

### Ex.144: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-2-methylbenzoic acid

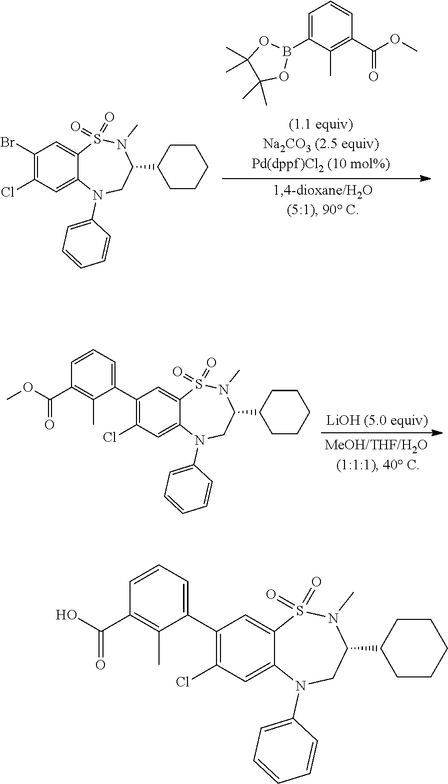

Step 1

A mixture of (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 eq), methyl 2-methyl-3-(4,4,5,5-tetramethyl-1,3,2-dioxaborolan-2-yl)benzoate (63 mg, 0.23 mmol, 1.1 eq), Pd(dppf)Cl₂ (15 mg, 0.02 mmol, 0.1 eq) and Na₂CO₃ (56 mg, 0.53 mmol, 2.5 eq) in 1,4-dioxane (3 mL) and H₂O (0.6 mL) was stirred for 1 h at 90° C. under N₂ atmosphere. The mixture was allowed to cool to room temperature and poured into water (20 mL). The resulting mixture was extracted with CH₂C₁₂ (20 mL×3). The combined organic layers were dried over anhydrous Na₂SO₄ and concentrated under reduced pressure. The residue was purified by silica gel column chromatography (eluting with 10/90=EtOAc/petroleum ether) to give methyl (R)-3-(7-chloro-3-cyclohexyl-2-methyl-1,1-dioxido-5-phenyl-2,3,4,5-tetrahydrobenzo[f][1,2,5]thiadiazepin-8-yl)-2-methylbenzoate (90 mg, 79% yield) as a white solid. ESI MS m/z=553.0 [M+H]⁺.

Step 2

A mixture of methyl (R)-3-(7-chloro-3-cyclohexyl-2-methyl-1,1-dioxido-5-phenyl-2,3,4,5-tetrahydrobenzo[f][1,2,5]thiadiazepin-8-yl)-2-methylbenzoate (90 mg, 0.16 mmol, 1.0 eq) and LiOH (33 mg, 0.79 mmol, 5.0 eq) in MeOH (0.4 mL), H₂O (0.4 mL) and THE (0.4 mL) was stirred for 1 h at 40° C. The mixture was allowed to cool down to room temperature and poured into water (10 mL). The resulting mixture was extracted with EtOAc (20 mL×3). The combined organic layers were dried over anhydrous Na₂SO₄ and concentrated under reduced pressure. The residue was purified by RPHPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)-2-methylbenzoic acid, Ex.144 (24.3 mg, 28% yield) as a white solid. ESI MS m/z=539.2 [M+H]⁺. ¹H NMR (300 MHz, DMSO-d₆): δ 13.12 (brs, 1H), 7.80 (d, J=6.3 Hz, 1H), 7.62 (d, J=3.9 Hz, 1H), 7.50-7.16 (m, 5H), 7.15-6.80 (m, 3H), 4.54-4.20 (m, 1H), 3.64 (s, 1H), 3.33 (m, 1H), 2.67 (s, 3H), 2.29-2.22 (m, 3H), 2.13-1.83 (m, 2H), 1.81-1.42 (m, 4H), 1.42-1.10 (m, 3H), 1.10-0.70 (m, 2H).

| Example | Structure | MS | NMR |
|---|---|---|---|
| 144 | 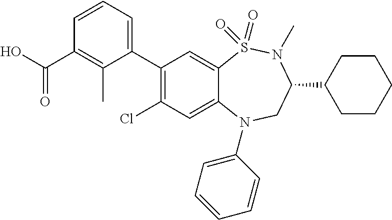 [SMILES: Cc1c(C(=O)O)cccc1-c1cc2c(cc1Cl)N(c1ccccc1)C[C@@H](C1CCCCC1)N(C)S2(=O)=O] | [M + H]⁺ 539.2 | ¹H NMR (300 MHz, DMSO-d₆): δ 13.12 (brs, 1H), 7.80 (d, J = 6.3 Hz, 1H), 7.62 (d, J = 3.9 Hz, 1H), 7.50- 7.16 (m, 5H), 7.15-6.80 (m, 3H), 4.54-4.20 (m, 1H), 3.64 (s, 1H), 3.33 (m, 1H), 2.67 (s, 3H), 2.29- 2.22 (m, 3H), 2.13-1.83 (m, 2H), 1.81-1.42 (m, 4H), 1.42-1.10 (m, 3H), 1.10-0.70 (m, 2H). |

### Ex.145: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-4-methylbenzoic acid

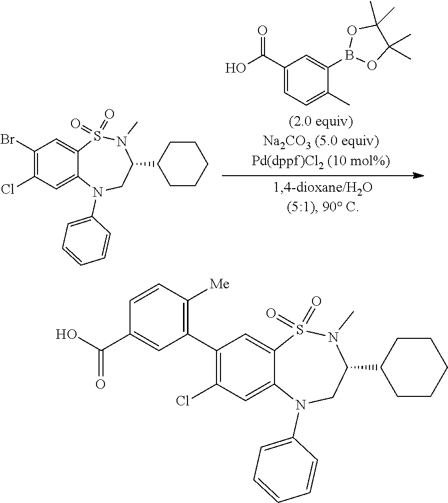 [SMILES: Cc1ccc(C(=O)O)cc1B1OC(C)(C)C(C)(C)O1 ; CN1[C@H](C2CCCCC2)CN(c2ccccc2)c2cc(Cl)c(Br)cc2S1(=O)=O ; Cc1ccc(C(=O)O)cc1-c1cc2c(cc1Cl)N(c1ccccc1)C[C@@H](C1CCCCC1)N(C)S2(=O)=O]

Step 1

A mixture of 4-methyl-3-(4,4,5,5-tetramethyl-1,3,2-dioxaborolan-2-yl)benzoic acid (109 mg, 0.41 mmol, 2.0 eq), (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 eq), Na₂CO₃ (110 mg, 1.04 mmol, 5.0 eq) and Pd(dppf)Cl₂ (15 mg, 0.02 mmol, 0.1 eq) in 1,4-dioxane (5 mL) and H₂O (1 mL) was stirred for 2 h at 90° C. under a nitrogen atmosphere. The resulting mixture was cooled to room temperature. The reaction was quenched with H₂O (20 mL) at room temperature, and the resulting mixture was extracted three times with ethyl acetate (3×20 mL). The combined organic layers were dried over anhydrous Na₂SO₄. The residue was purified by RPHPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)-4-methylbenzoic acid as a white solid (20.7 mg, 18% yield). ESI MS m/z=539.0 [M+H]⁺. ¹H NMR (400 MHz, DMSO-d₆): δ 13.07 (brs, 1H), 7.92 (d, J=7.8 Hz, 1H), 7.76-7.70 (m, 1H), 7.65 (d, J=8.0 Hz, 1H), 7.48 (t, J=7.2 Hz, 1H), 7.37-7.35 (m, 3H), 7.09-6.89 (m, 3H), 4.47-4.27 (m, 1H), 3.92-3.38 (m, 2H), 2.68 (d, J=10.4 Hz, 3H), 2.22-2.16 (m, 3H), 1.98-1.92 (m, 2H), 1.72 (s, 2H), 1.61 (s, 2H), 1.40-1.06 (m, 3H), 1.05-0.78 (m, 2H).

| Example | Structure | MS | NMR |
|---|---|---|---|
| 145 | 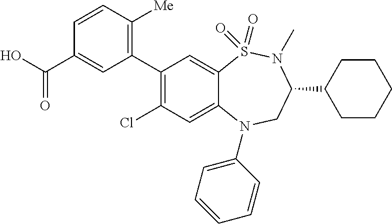 [SMILES: Cc1ccc(C(=O)O)cc1-c1cc2c(cc1Cl)N(c1ccccc1)C[C@@H](C1CCCCC1)N(C)S2(=O)=O] | [M + H]⁺ 539.0 | ¹H NMR (400 MHz, DMSO-d₆): δ 13.07 (brs, 1H), 7.92 (d, J = 7.8 Hz, 1H), 7.76-7.70 (m, 1H), 7.65 (d, J = 8.0 Hz, 1H), 7.48 (t, J = 7.2 Hz, 1H), 7.37-7.35 (m, 3H), 7.09-6.89 (m, 3H), 4.47-4.27 (m, 1H), 3.92- 3.38 (m, 2H), 2.68 (d, J = 10.4 Hz, 3H), 2.22-2.16 (m, 3H), 1.98- 1.92 (m, 2H), 1.72 (s, 2H), 1.61 (s, 2H), 1.40-1.06 (m, 3H), 1.05- 0.78 (m, 2H) |

### Ex. 146 and Ex.147: Synthesis of 3-((3R)-7-chloro-3-cyclohexyl-2-methyl-1,1-dioxido-5-phenyl-2,3,4,5-tetrahydrobenzo[f][1,2,5]thiadiazepin-8-yl)-2,4-dimethylbenzoic acid

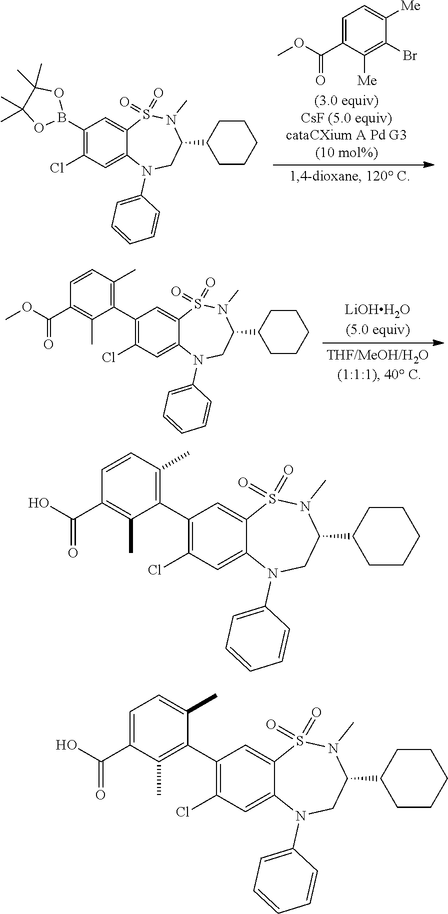

Step 1

A mixture of cataCXium A Pd G3 (42.0 mg, 0.06 mmol, 0.1 equiv, CAS #1651823-59-4), methyl 3-bromo-2,4-dimethylbenzoate (400.0 mg, 1.65 mmol, 3.0 eq.), (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (300.0 mg, 0.57 mmol, 1.0 eq.) and CsF (430.0 mg, 2.83 mmol, 5.0 eq.) in 1,4-dioxane (10.0 mL) was heated under microwave irradiation at 120° C. for 40 min. The reaction was quenched with water (10 mL). The resulting mixture was then extracted three times with EtOAc (20 mL each). The combined organic layers were dried over anhydrous Na₂SO₄ and concentrated under reduced pressure. The residue was purified by silica gel column chromatography (eluting with EtOAc/Petroleum ether=30/70) to give methyl 3-((3R)-7-chloro-3-cyclohexyl-2-methyl-1,1-dioxido-5-phenyl-2,3,4,5-tetrahydrobenzo[f][1,2,5]thiadiazepin-8-yl)-2,4-dimethylbenzoate (130.0 mg, 41% yield) as a white solid. ESI MS m/z=567.0 [M+H]⁺.

Step 2

A mixture of methyl 3-((3R)-7-chloro-3-cyclohexyl-2-methyl-1,1-dioxido-5-phenyl-2,3,4,5-tetrahydrobenzo[f][1,2,5]thiadiazepin-8-yl)-2,4-dimethylbenzoate (130.0 mg, 0.23 mmol, 1.0 eq.) and LiOH H₂O (53.0 mg, 1.27 mmol, 5.0 eq.) in MeOH (0.5 mL), THE (0.5 mL) and H₂O (0.5 mL) was stirred for 2 h at 40° C. The resulting mixture was concentrated under vacuum. The residue was adjusted to pH 6 with HCl (1N). The resulting mixture was extracted three times with EtOAc (20 mL each). The combined organic layers were dried over anhydrous Na₂SO₄ and concentrated under reduced pressure. The residue was purified by RPHIPLC to afford Ex. 146 (8.9 mg, 7% yield) and Ex. 147 (10.8 mg, 8% yield) as white solids. The products of this reaction were assigned as atropisomers and the relative configuration was not determined. ESI MS m/z=550.9 [M+H]⁺.

| Example | Structure | MS | NMR |
|---|---|---|---|
| 146 | 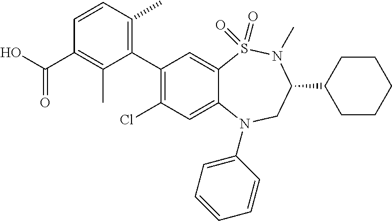 | [M + H]⁺ 550.9 | ¹H NMR (400 MHz, DMSO-d₆): δ 12.56 (s, 1H), 7.77 (d, J = 8.0 Hz, 1H), 7.55 (s, 1H), 7.42 (s, 1H), 7.37- 7.23 (m, 3H), 6.93 (s, 3H), 4.35 (d, J = 15.2 Hz, 1H), 3.75-3.49 (m, 2H), 2.63 (s, 3H), 2.15 (s, 3H), 2.03 (d, J = 12.4 Hz, 3H), 2.01-1.95 (m, 1H), 1.90-1.82 (m, 1H), 1.76-1.56 (m, 4H), 1.26-0.99 (m, 5H) |
| 147 | 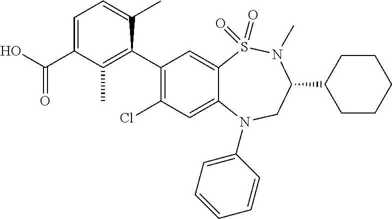 | [M + H]⁺ 550.9 | ¹H NMR (400 MHz, DMSO-d₆): δ 12.73 (s, 1H), 7.77 (d, J = 8.0 Hz, 1H), 7.55 (s, 1H), 7.43 (s, 1H), 7.35- 7.25 (m, 3H), 6.99-7.89 (m, 3H), 4.35 (d, J = 15.2 Hz, 1H), 3.86-3.46 (m, 2H), 2.63 (s, 3H), 2.22 (s, 3H), 1.99-1.98 (m, 1H), 1.97 (s, 3H), 1.90-1.86 (m, 1H), 1.80-1.55 (m, 4H), 1.22-0.98 (m, 5H) |

The following compound was prepared using a procedure analogous to that used for Ex. 144:

| Example | Structure | MS | NMR |
|---|---|---|---|
| 148 | 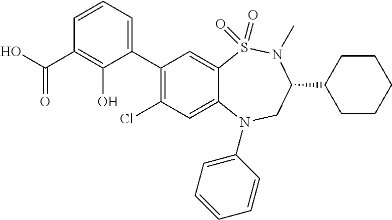 [SMILES: CN1[C@H](C2CCCCC2)CN(c2ccccc2)c2cc(Cl)c(-c3cccc(C(=O)O)c3O)cc2S1(=O)=O] | [M + H]⁺ 541.0 | ¹H NMR (300 MHz, DMSO-d₆): δ 7.77-7.70 (m, 2H), 7.34-7.23 (m, 3H), 7.16 (dd, J = 7.5, 1.8 Hz, 1H), 6.91 (s, 3H), 6.65 (t, J = 7.5 Hz, 1H), 4.36 (d, J = 15.0 Hz, 1H), 3.57 (s, 1H), 3.36 (d, J = 5.7 Hz, 1H), 2.63 (s, 3H), 2.07-1.82 (m, 2H), 1.81-1.54 (m, 4H), 1.31-1.14 (m, 3H), 1.09-0.88 (m, 2H) |

### Ex. 149: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-2-fluorobenzoic acid

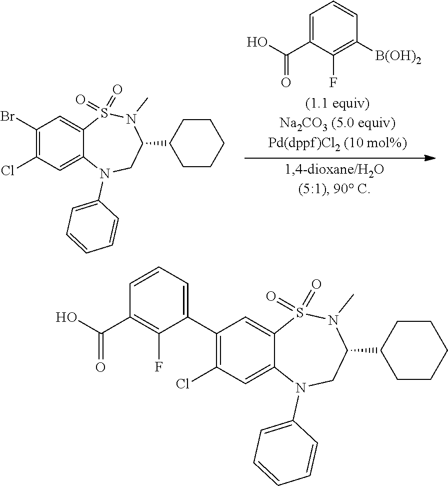 [SMILES: O=C(O)c1cccc(B(O)O)c1F ; CN1[C@H](C2CCCCC2)CN(c2ccccc2)c2cc(Cl)c(Br)cc2S1(=O)=O ; CN1[C@H](C2CCCCC2)CN(c2ccccc2)c2cc(Cl)c(-c3cccc(C(=O)O)c3F)cc2S1(=O)=O]

Step 1

A mixture of 3-borono-2-fluorobenzoic acid (115 mg, 0.63 mmol, 1.1 equiv), (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 eq), Na₂CO₃ (111 mg, 1.05 mmol, 5.0 eq) and Pd(dppf)C₁₂ (16 mg, 0.02 mmol, 0.1 eq) in 1,4-dioxane (3 mL) and H₂O (0.6 mL) was stirred for 2 h at 90° C. under nitrogen atmosphere. The resulting mixture was cooled to room temperature. The reaction was quenched with H₂O (20 mL) at room temperature. The resulting mixture was extracted three times with EtOAc (20 mL each). The combined organic layers were dried over anhydrous Na₂SO₄ and concentrated under reduced pressure. The residue was purified by RPHPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)-2-fluorobenzoic acid, Ex.149 (23.5 mg, 21.4%) as a white solid. ESI MS m/z=543.0 [M+H]⁺. ¹H NMR (300 MHz, DMSO): δ 13.4 (s, 1H), 7.93 (t, J=6.6 Hz, 1H), 7.82-7.59 (m, 2H), 7.44-7.21 (m, 4H), 7.09-6.96 (m, 3H), 4.35 (d, J=15.3 Hz, 1H), 3.78 (s, 1H), 3.40-3.32 (m, 1H), 2.72 (s, 3H), 2.02-1.82 (m, 2H), 1.79-1.51 (m, 4H), 1.32-1.06 (m, 3H), 1.09-0.92 (m, 2H).

| Example | Structure | MS | NMR |
|---|---|---|---|
| 149 | 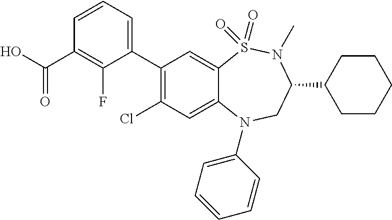 [SMILES: CN1[C@H](C2CCCCC2)CN(c2ccccc2)c2cc(Cl)c(-c3cccc(C(=O)O)c3F)cc2S1(=O)=O] | [M + H]⁺ 543.0 | ¹H NMR (300 MHz, DMSO): δ 13.4 (s, 1H), 7.93 (t, J = 6.6 Hz, 1H), 7.82-7.59 (m, 2H), 7.44-7.21 (m, 4H), 7.09-6.96 (m, 3H), 4.35 (d, J = 15.3 Hz, 1H), 3.78 (s, 1H), 3.40- 3.32 (m, 1H), 2.72 (s, 3H), 2.02- 1.82 (m, 2H), 1.79-1.51 (m, 4H), 1.32-1.06 (m, 3H), 1.09-0.92 (m, 2H) |

### Ex.150: Synthesis of (R)-3-(7-chloro-3-cyclohexyl-2-methyl-1,1-dioxido-5-phenyl-2,3,4,5-tetrahydrobenzo[f][1,2,5]thiadiazepin-8-yl)-4-fluorobenzoic acid

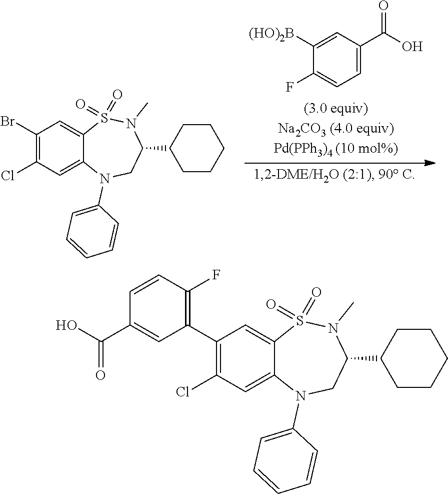 [SMILES: O=C(O)c1ccc(F)c(B(O)O)c1 ; CN1[C@H](C2CCCCC2)CN(c2ccccc2)c2cc(Cl)c(Br)cc2S1(=O)=O ; CN1[C@H](C2CCCCC2)CN(c2ccccc2)c2cc(Cl)c(-c3cc(C(=O)O)ccc3F)cc2S1(=O)=O]

Step 1

A mixture of 3-borono-4-fluorobenzoic acid (114 mg, 0.62 mmol, 3.0 eq), (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 eq), Na₂CO₃ (88 mg, 0.83 mmol, 4.0 eq) and Pd(PPh₃)₄ (24 mg, 0.02 mmol, 0.1 eq) in H₂O (0.5 mL) and DME (2.0 mL) was stirred for 2 h at 80° C. under a nitrogen atmosphere. The resulting mixture was cooled to room temperature. The reaction was quenched with H₂O (20 mL) at room temperature. The resulting mixture was extracted three times with EtOAc (20 mL each). The combined organic layers were dried over anhydrous Na₂SO₄. The residue was then purified by RPHPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)-4-fluorobenzoic acid, Ex.150 (20.4 mg, 18% yield) as a white solid. ESI MS m/z=543.0 [M+H]⁺. ¹H NMR (300 MHz, DMSO-d₆): δ 8.12-7.90 (m, 2H), 7.76 (s, 1H), 7.52-7.15 (m, 4H), 7.06-6.98 (m, 3H), 4.44-4.28 (m, 1H), 3.36-3.32 (m, 2H), 2.73 (s, 3H), 2.01-1.82 (m, 2H), 1.69-1.60 (m, 4H), 1.31-1.08 (m, 3H), 0.98-0.94 (m, 2H).

| Example | Structure | MS | NMR |
|---|---|---|---|
| 150 | 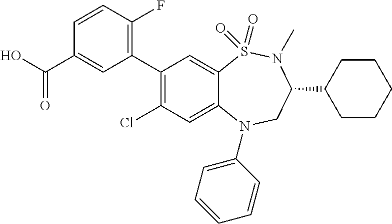 [SMILES: CN1[C@H](C2CCCCC2)CN(c2ccccc2)c2cc(Cl)c(-c3cc(C(=O)O)ccc3F)cc2S1(=O)=O] | [M + H]⁺ 543.0 | δ 8.12-7.90 (m, 2H), 7.76 (s, 1H), 7.52-7.15 (m, 4H), 7.06-6.98 (m, 3H), 4.44-4.28 (m, 1H), 3.36- 3.32 (m, 2H), 2.73 (s, 3H), 2.01- 1.82 (m, 2H), 1.69-1.60 (m, 4H), 1.31-1.08 (m, 3H), 0.98-0.94 (m, 2H) |

### Ex.151: Synthesis of (R)-4-chloro-3-(7-chloro-3-cyclohexyl-2-methyl-1,1-dioxido-5-phenyl-2,3,4,5-tetrahydrobenzo[f][1,2,5]thiadiazepin-8-yl)benzoic acid

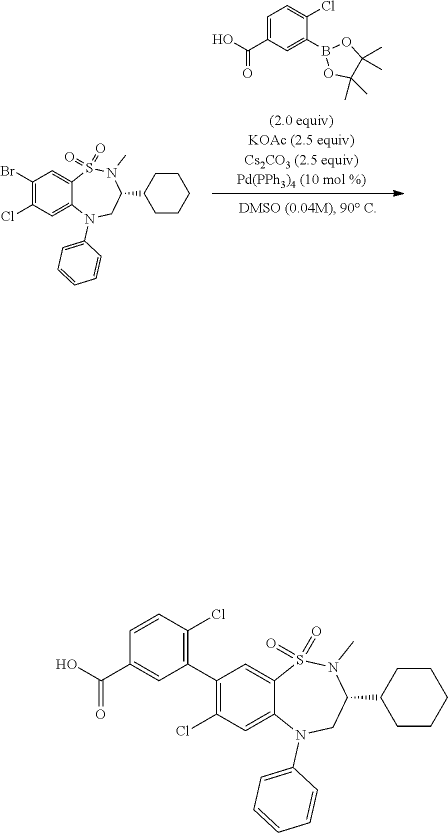 [SMILES: CC1(C)OB(c2cc(C(=O)O)ccc2Cl)OC1(C)C ; CN1[C@H](C2CCCCC2)CN(c2ccccc2)c2cc(Cl)c(Br)cc2S1(=O)=O ; CN1[C@H](C2CCCCC2)CN(c2ccccc2)c2cc(Cl)c(-c3cc(C(=O)O)ccc3Cl)cc2S1(=O)=O]

Step 1

A mixture of 4-chloro-3-(4,4,5,5-tetramethyl-1,3,2-dioxaborolan-2-yl)benzoic acid (117 mg, 0.41 mmol, 2.0 eq), (R)-8-bromo-7-chloro-3-cyclohexyl-2-methyl-5-phenyl-2,3,4,5-tetrahydrobenzo[f][1,2,5]thiadiazepine 1,1-dioxide (100 mg, 0.21 mmol, 1.0 eq), Pd(PPh₃)₄ (23 mg, 0.02 mmol, 0.1 eq), potassium acetate (51 mg, 0.52 mmol, 2.5 eq) and Cs₂CO₃ (171 mg, 0.53 mmol, 2.5 eq) in DMSO (5 mL) was stirred for 4 h at 90° C. under nitrogen atmosphere. The resulting mixture was cooled to room temperature. The reaction was quenched with water (10 mL). The resulting mixture was extracted three times with EtOAc (20 mL each). The combined organic layers were dried over anhydrous Na₂SO₄ and concentrated. The residue was purified by silica gel column chromatography (eluting with DCM/MeOH=10/1). The crude product was further purified by RPHPLC to afford (R)-4-chloro-3-(7-chloro-3-cyclohexyl-2-methyl-1,1-dioxido-5-phenyl-2,3,4,5-tetrahydrobenzo[f][1,2,5]thiadiazepin-8-yl)benzoic acid, Ex. 151 (19.2 mg, 16% yield) as a white solid. ESI MS m/z=558.9 [M+H]⁺. ¹H NMR (300 MHz, DMSO-d₆): δ 8.25 (s, 1H), 8.05-7.80 (m, 2H), 7.77-7.55 (m, 2H), 7.46-7.15 (m, 3H), 7.15-6.79 (m, 3H), 4.50-4.24 (m, 1H), 4.10-2.85 (m, 2H), 2.72-2.68 (m, 3H), 2.08-1.82 (m, 2H), 1.80-1.43 (m, 4H), 1.40-1.10 (m, 3H), 1.10-0.90 (m, 2H).

| Example | Structure | MS | NMR |
|---|---|---|---|
| 151 | 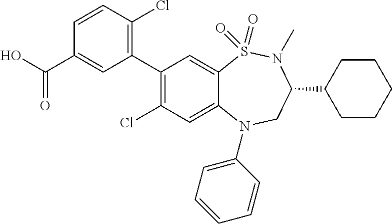 [SMILES: CN1[C@H](C2CCCCC2)CN(c2ccccc2)c2cc(Cl)c(-c3cc(C(=O)O)ccc3Cl)cc2S1(=O)=O] | [M + H]⁺ 558.9 | ¹H NMR (300 MHz, DMSO-d₆): δ 8.25 (s, 1H), 8.05-7.80 (m, 2H), 7.77-7.55 (m, 2H), 7.46-7.15 (m, 3H), 7.15-6.79 (m, 3H), 4.50- 4.24 (m, 1H), 4.10-2.85 (m, 2H), 2.72-2.68 (m, 3H), 2.08-1.82 (m, 2H), 1.80-1.43 (m, 4H), 1.40- 1.10 (m, 3H), 1.10-0.90 (m, 2H) |

### Ex.152: Synthesis of(R)-2-chloro-3-(7-chloro-3-cyclohexyl-2-methyl-1,1-dioxido-5-phenyl-2,3,4,5-tetrahydrobenzo[f][1,2,5]thiadiazepin-8-yl)benzoic acid

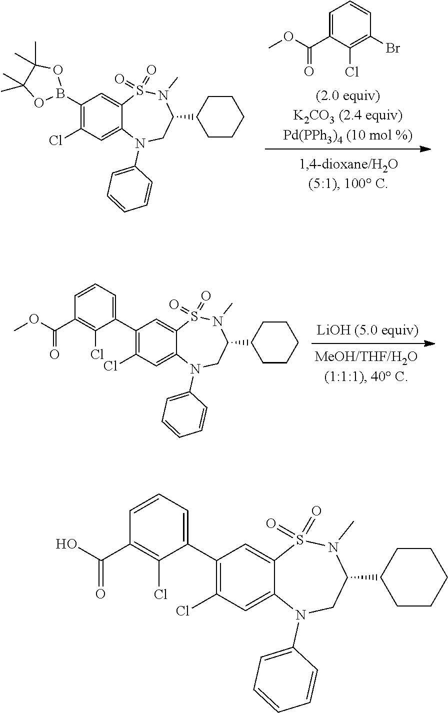 [SMILES: CN1[C@H](C2CCCCC2)CN(c2ccccc2)c2cc(Cl)c(B3OC(C)(C)C(C)(C)O3)cc2S1(=O)=O ; COC(=O)c1cccc(Br)c1Cl ; COC(=O)c1cccc(-c2cc3c(cc2Cl)N(c2ccccc2)C[C@@H](C2CCCCC2)N(C)S3(=O)=O)c1Cl ; CN1[C@H](C2CCCCC2)CN(c2ccccc2)c2cc(Cl)c(-c3cccc(C(=O)O)c3Cl)cc2S1(=O)=O]

Step 1

A mixture of methyl 3-bromo-2-chlorobenzoate (94 mg, 0.38 mmol, 2.0 eq), (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (100 mg, 0.19 mmol, 1.0 eq), Pd(PPh₃)₄ (22 mg, 0.02 mmol, 0.1 eq) and K₂CO₃ (65 mg, 0.47 mmol, 2.4 eq) in 1,4-dioxane (5 mL) and H₂O (1 mL) was stirred for overnight at 100° C. under nitrogen atmosphere. The resulting mixture was cooled to room temperature. The reaction was quenched with H₂O (20 mL). The resulting mixture was extracted three times with EtOAc (25 mL each). The combined organic layers were dried over anhydrous Na₂SO₄. The residue was purified by silica gel column chromatography (eluting with petroleum ether/EtOAc=5/1) to give methyl (R)-2-chloro-3-(7-chloro-3-cyclohexyl-2-methyl-1,1-dioxido-5-phenyl-2,3,4,5-tetrahydrobenzo[f][1,2,5]thiadiazepin-8-yl)benzoate (99 mg, 92% yield) as a colorless oil. ESI MS m/z=572.9 [M+H]⁺.

Step 2

A mixture of methyl (R)-2-chloro-3-(7-chloro-3-cyclohexyl-2-methyl-1,1-dioxido-5-phenyl-2,3,4,5-tetrahydrobenzo[f][1,2,5]thiadiazepin-8-yl)benzoate (115 mg, 0.2 mmol, 1.0 eq) and LiOH (42 mg, 1.0 mmol, 5.0 eq) in MeOH (2.0 mL), H₂O (2 mL) and THE (2 mL) was stirred for 3 h at 40° C. The volatiles were removed under reduced pressure and the residue was adjusted to pH 6 with HCl (1N). The resulting precipitate was collected via filtration and lyophilized from acetonitrile and water to obtain (R)-2-chloro-3-(7-chloro-3-cyclohexyl-2-methyl-1,1-dioxido-5-phenyl-2,3,4,5-tetrahydrobenzo[f][1,2,5]thiadiazepin-8-yl)benzoic acid, Ex.152 (40 mg, 36% yield) as a white solid. ESI MS m/z=558.9 [M+H]⁺. ¹H NMR (400 MHz, DMSO-d₆): δ 13.66 (brs, 1H), 7.74 (d, J=6.8 Hz, 1H), 7.66 (d, J=6.0 Hz, 1H), 7.60-7.42 (m, 2H), 7.42-7.17 (m, 3H), 7.17-6.70 (m, 3H), 4.35 (d, J=15.2 Hz, 1H), 3.72 (s, 1H), 3.33-3.24 (m, 1H), 2.70 (s, 3H), 2.25-1.82 (m, 2H), 1.82-1.48 (m, 4H), 1.42-1.07 (m, 3H), 1.06-0.90 (m, 2H).

| Example | Structure | MS | NMR |
|---|---|---|---|
| 152 | 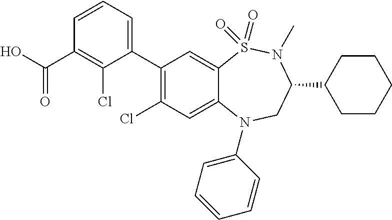 [SMILES: CN1[C@H](C2CCCCC2)CN(c2ccccc2)c2cc(Cl)c(-c3cccc(C(=O)O)c3Cl)cc2S1(=O)=O] | [M + H]⁺ 558.9 | ¹H NMR (400 MHz, DMSO-d₆): δ 13.66 (brs, 1H), 7.74 (d, J = 6.8 Hz, 1H), 7.66 (d, J = 6.0 Hz, 1H), 7.60- 7.42 (m, 2H), 7.42-7.17 (m, 3H), 7.17-6.70 (m, 3H), 4.35 (d, J = 15.2 Hz, 1H), 3.72 (s, 1H), 3.33- 3.24 (m, 1H), 2.70 (s, 3H), 2.25- 1.82 (m, 2H), 1.82-1.48 (m, 4H), 1.42-1.07 (m, 3H), 1.06-0.90 (m, 2H) |

### Ex.153: Synthesis of (R)-6-(7-chloro-3-cyclohexyl-2-methyl-1,1-dioxido-5-phenyl-2,3,4,5-tetrahydrobenzo[f][1,2,5]thiadiazepin-8-yl)benzo[d][1,3]dioxole-4-carboxylic acid

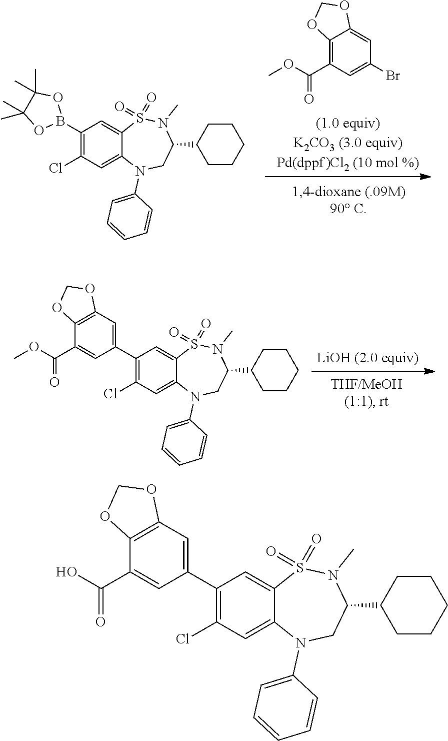 [SMILES: CN1[C@H](C2CCCCC2)CN(c2ccccc2)c2cc(Cl)c(B3OC(C)(C)C(C)(C)O3)cc2S1(=O)=O ; COC(=O)c1cc(Br)cc2c1OCO2 ; COC(=O)c1cc(-c2cc3c(cc2Cl)N(c2ccccc2)C[C@@H](C2CCCCC2)N(C)S3(=O)=O)cc2c1OCO2 ; CN1[C@H](C2CCCCC2)CN(c2ccccc2)c2cc(Cl)c(-c3cc4c(c(C(=O)O)c3)OCO4)cc2S1(=O)=O]

Step 1

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (753 mg, 1.4 mmol, 1.5 eq) and methyl 6-bromobenzo[d][1,3]dioxole-4-carboxylate (245 mg, 0.9 mmol 1.0 eq) in 1,4-dioxane (10 mL) were added K₂CO₃ (392 mg, 2.8 mmol, 3.0 eq) and Pd(dppf)C₁₂ (69 mg, 0.09 mmol, 0.1 eq) under N₂. The reaction mixture was heated at 90° C. overnight. Upon completion, as determined by LCMS analysis, the reaction mixture was cooled to rt and poured into water. The aqueous phase was extracted with EtOAc (30 mL). The organic phase was concentrated, and the residue was purified by RPHPLC to afford methyl (R)-6-(7-chloro-3-cyclohexyl-2-methyl-1,1-dioxido-5-phenyl-2,3,4,5-tetrahydrobenzo[f][1,2,5]thiadiazepin-8-yl)benzo[d][1,3]dioxole-4-carboxylate (380 mg, 69% yield) as a white solid. ESI MS m/z=583.0 [M+H]⁺. ¹H NMR (400 MHz, CDCl₃): δ 7.72 (s, 1H), 7.37-7.27 (m, 5H), 6.98 (d, J=6.8 Hz, 3H), 6.25 (s, 2H), 4.36 (d, J=14.4 Hz, 1H), 3.92 (s, 3H), 3.78-3.60 (m, 1H), 3.30 (m, 1H), 2.69 (s, 3H), 2.02-1.84 (m, 2H), 1.78-1.68 (m, 2H), 1.62-1.50 (m, 2H), 1.27-1.17 (m, 5H).

Step 2

To a solution of methyl (R)-6-(7-chloro-3-cyclohexyl-2-methyl-1,1-dioxido-5-phenyl-2,3,4,5-tetrahydrobenzo[f][1,2,5]thiadiazepin-8-yl)benzo[d][1,3]dioxole-4-carboxylate (360 mg, 0.6 mmol 1.0 eq) in MeOH (2 mL) and THE (2 mL) was added lithium hydroxide (50 mg, 1.2 mmol, 2.0 eq). The reaction mixture was stirred at room temperature overnight. The resulting mixture was concentrated under vacuum. The aqueous phase was acidified with 1 N HCl, and the resulting precipitate was collected via filtration and dried to obtain (R)-6-(7-chloro-3-cyclohexyl-2-methyl-1,1-dioxido-5-phenyl-2,3,4,5-tetrahydrobenzo[f][1,2,5]thiadiazepin-8-yl)benzo[d][1,3]dioxole-4-carboxylic acid, Ex.153 (50 mg, 14% yield) as a white solid. ESI MS m/z=566.9 [M+H]⁺. ¹H NMR (400 MHz, DMSO-d₆): δ 13.18 (s, 1H), 7.72 (s, 1H), 7.37-7.21 (m, 5H), 7.12-6.84 (m, 3H), 6.22 (s, 2H), 4.36 (d, J=15.6 Hz, 1H), 3.65 (s, 1H), 3.35 (s, 1H), 2.68 (s, 3H), 2.02-1.82 (m, 2H), 1.75-1.55 (m, 4H), 1.27-1.10 (m, 3H), 1.02-0.92 (m, 2H).

| Example | Structure | MS | NMR |
|---|---|---|---|
| 153 | 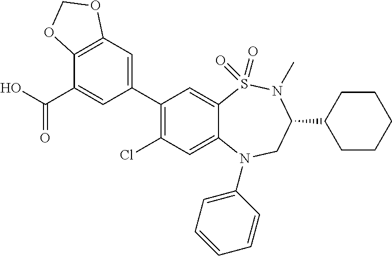 [SMILES: CN1[C@H](C2CCCCC2)CN(c2ccccc2)c2cc(Cl)c(-c3cc4c(c(C(=O)O)c3)OCO4)cc2S1(=O)=O] | [M + H]⁺ 566.9 | ¹H NMR (400 MHz, DMSO-d₆): δ 13.18 (s, 1H), 7.72 (s, 1H), 7.37- 7.21 (m, 5H), 7.12-6.84 (m, 3H), 6.22 (s, 2H), 4.36 (d, J = 15.6 Hz, 1H), 3.65 (s, 1H), 3.35 (s, 1H), 2.68 (s, 3H), 2.02-1.82 (m, 2H), 1.75- 1.55 (m, 4H), 1.27-1.10 (m, 3H), 1.02-0.92 (m, 2H) |

### Ex.154: Synthesis of (R)-6-(7-chloro-3-cyclohexyl-2-methyl-1,1-dioxido-5-phenyl-2,3,4,5-tetrahydrobenzo[f][1,2,5]thiadiazepin-8-yl)-2,2-difluorobenzo[d][1,3]dioxole-4-carboxylic acid

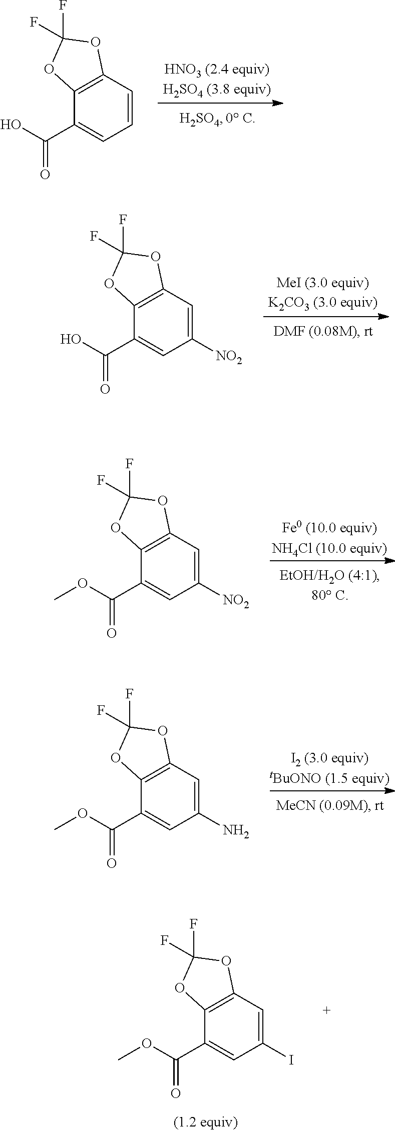 [SMILES: O=C(O)c1cccc2c1OC(F)(F)O2 ; O=C(O)c1cc([N+](=O)[O-])cc2c1OC(F)(F)O2 ; COC(=O)c1cc([N+](=O)[O-])cc2c1OC(F)(F)O2 ; COC(=O)c1cc(N)cc2c1OC(F)(F)O2 ; COC(=O)c1cc(I)cc2c1OC(F)(F)O2]

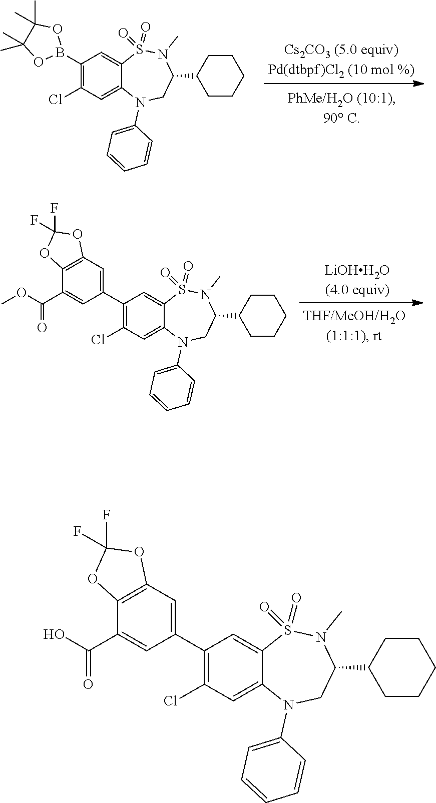 [SMILES: CN1[C@H](C2CCCCC2)CN(c2ccccc2)c2cc(Cl)c(B3OC(C)(C)C(C)(C)O3)cc2S1(=O)=O ; COC(=O)c1cc(-c2cc3c(cc2Cl)N(c2ccccc2)C[C@@H](C2CCCCC2)N(C)S3(=O)=O)cc2c1OC(F)(F)O2 ; CN1[C@H](C2CCCCC2)CN(c2ccccc2)c2cc(Cl)c(-c3cc4c(c(C(=O)O)c3)OC(F)(F)O4)cc2S1(=O)=O]

Step 1

To a solution of 2,2-difluorobenzo[d][1,3]dioxole-4-carboxylic acid (2.0 g, 9.9 mmol, 1.0 eq) in H₂SO₄ (8 mL) was added H₂SO₄ (2 mL, 3.8 mmol, 3.8 eq) and HNO₃ (1 mL, 2.4 mmol, 2.4 eq) dropwise at 0° C. The reaction mixture was stirred at 0° C. for 1 h. The reaction was then added to ice water and stirred for 20 minutes. The mixture was filtered, and the filter cake was concentrated to give 2,2-difluoro-6-nitrobenzo[d][1,3]dioxole-4-carboxylic acid (1.0 g, 41% yield) as a white solid. ESI MS m/z=245.9 [M−H]. ¹H NMR (300 MHz, CDCl₃): δ 8.72 (d, J=2.1 Hz, 1H), 8.36-8.09 (m, 1H).

Step 2

To a solution of 2,2-difluoro-6-nitrobenzo[d][1,3]dioxole-4-carboxylic acid (1.0 g, 4.0 mmol, 1.0 eq) in DMF (50 mL) was added K₂CO₃ (1.7 g, 12.3 mmol, 3.0 eq) and CH₃I (1.8 g, 12.7 mmol, 3.0 eq). The reaction mixture was then stirred at room temperature for 2 h. The mixture was concentrated, diluted with water (100 mL) and extracted three times with EtOAc (100 mL each). The combined organic layers were washed with brine (100 mL), dried over Na₂SO₄ and concentrated to give a residue which was purified by silica gel chromatography (eluted with petroleum ether: EtOAc=100/1) to give methyl 2,2-difluoro-6-nitrobenzo[d][1,3]dioxole-4-carboxylate (1.0 g, 95% yield) as a white solid. ESI MS m/z=261.9 [M+H]⁺. ¹H NMR (400 MHz, DMSO-d₆): δ 8.67 (d, J=2.4 Hz, 1H), 8.45 (d, J=2.4 Hz, 1H), 3.96 (s, 3H).

Step 3

To a solution of methyl 2,2-difluoro-6-nitrobenzo[d][1,3]dioxole-4-carboxylate (1.0 g, 3.8 mmol, 1.0 eq) in EtOH/H₂O (40/10 mL) was added NH₄C₁ (2.0 g, 38.3 mmol, 10.0 eq) and Fe (2.1 g, 38.3 mmol, 10.0 eq). The reaction mixture was then stirred at 80° C. for 2 h. The mixture was filtered, and the filtrate was concentrated to give a crude residue that was partitioned between EtOAc (50 mL) and water (25 mL). The layers were separated, and the aqueous layer was extracted twice with EtOAc (50 mL each). The combined organic layers were washed with H₂O (50 mL) and brine (50 mL) before being dried over Na₂SO₄. Concentration gave methyl 6-amino-2,2-difluorobenzo[d][1,3]dioxole-4-carboxylate (790 mg, 89% yield) as a yellow solid that was used without further purification. ESI MS m/z=232.0 [M+H]⁺. ¹H NMR (400 MHz, DMSO-d₆): δ 6.84-6.78 (m, 2H), 5.56 (s, 2H), 3.84 (s, 3H).

Step 4

To a solution of methyl 6-amino-2,2-difluorobenzo[d][1,3]dioxole-4-carboxylate (620.0 mg, 2.7 mmol, 1.0 eq) in acetonitrile (30 mL) was added tert-butyl nitrite (414.8 mg, 4.0 mmol, 1.5 eq) and I2 (2.0 g, 8.1 mmol, 3.0 eq). The reaction mixture was stirred at room temperature for 1 h. The mixture was then filtered, and the filtrate was concentrated to give a crude residue that was partitioned between EtOAc (50 mL) and water (25 mL). The layers were separated, and the aqueous layer was extracted twice with EtOAc (50 mL each). The combined organic layers were washed with H₂O (50 mL), brine (50 mL), and dried over Na₂SO₄. The organic layer was concentrated, and the residue was purified by silica gel column chromatography (petroleum ether: EtOAc=30:1) to give methyl 2,2-difluoro-6-iodobenzo[d][1,3]dioxole-4-carboxylate (350.0 mg, 38% yield) as a yellow solid. ESI MS m/z=343.0 [M+H]⁺. ¹H NMR (300 MHz, CDCl₃): δ 8.02 (d, J=1.8 Hz, 1H), 7.54 (d, J=1.8 Hz, 1H), 3.96 (s, 3H).

Step 5

To a solution of (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (80.0 mg, 0.15 mmol, 1.0 eq) and methyl 2,2-difluoro-6-iodobenzo[d][1,3]dioxole-4-carboxylate (61.9 mg, 0.18 mmol, 1.2 eq) in toluene/H₂O (2 mL/0.2 mL) was added Cs₂CO₃ (245.9 mg, 0.75 mmol, 5.0 eq) and Pd(dtbpf)C₁₂ (9.7 mg, 0.015 mmol, 0.1 eq). The reaction mixture was then stirred at 90° C. for 2 h. The mixture was filtered, and the filtrate was concentrated to give a residue which was purified by silica gel column chromatography (Petroleum ether: EtOAc=100:1 to 50:1 to 30:1) to give methyl (R)-6-(7-chloro-3-cyclohexyl-2-methyl-1,1-dioxido-5-phenyl-2,3,4,5-tetrahydrobenzo[f][1,2,5]thiadiazepin-8-yl)-2,2-difluorobenzo[d][1,3]dioxole-4-carboxylate (70.0 mg, 75.0% yield) as a white solid. ESI MS m/z=619.2 [M+H]⁺. ¹H NMR (400 MHz, CDCl₃): δ 7.84 (s, 1H), 7.74 (d, J=1.6 Hz, 1H), 7.40-7.34 (m, 3H), 7.15-7.03 (m, 4H), 4.40-4.26 (m, 1H), 3.99 (s, 3H), 3.26-3.16 (m, 1H), 2.90 (s, 3H), 2.20-2.12 (m, 1H), 1.85-1.65 (m, 5H), 1.23-1.11 (m, 2H), 1.02-0.86 (m, 3H).

Step 6

To a solution of methyl (R)-6-(7-chloro-3-cyclohexyl-2-methyl-1,1-dioxido-5-phenyl-2,3,4,5-tetrahydrobenzo[f][1,2,5]thiadiazepin-8-yl)-2,2-difluorobenzo[d][1,3]dioxole-4-carboxylate (70 mg, 0.11 mmol, 1.0 eq) in THF/MeOH/H₂O (2/2/2 mL) was added LiOH H₂O (18.9 mg, 0.44 mmol, 4.0 eq). The reaction mixture was then stirred at rt for 6 h. The reaction mixture was concentrated under reduced pressure to give a residue. The mixture was adjusted to pH 4 with HCl (1 N), and the resulting precipitate was collected via filtration and the filtrate was washed with cold water and lyophilized to give (R)-6-(7-chloro-3-cyclohexyl-2-methyl-1,1-dioxido-5-phenyl-2,3,4,5-tetrahydrobenzo[f][1,2,5]thiadiazepin-8-yl)-2,2-difluorobenzo[d][1,3]dioxole-4-carboxylic acid, Ex.154 (27 mg, 39% yield) as a white solid. ESI MS m/z=605.2 [M+H]⁺. ¹H NMR (400 MHz, DMSO-d₆): δ 13.85 (s, 1H), 7.90 (s, 1H), 7.78 (s, 1H), 7.68 (s, 1H), 7.37-7.23 (m, 3H), 7.10-6.80 (m, 3H), 4.48-4.26 (m, 1H), 3.71 (s, 1H), 3.33 (s, 1H), 2.71 (s, 3H), 2.00-1.82 (m, 2H), 1.80-1.54 (m, 2H), 1.62 (s, 2H), 1.27-1.14 (m, 3H), 1.06-0.90 (m, 2H).

| Example | Structure | MS | NMR |
|---|---|---|---|
| 154 | 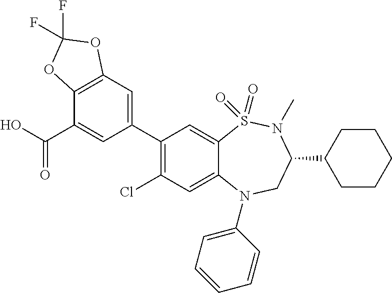 [SMILES: CN1[C@H](C2CCCCC2)CN(c2ccccc2)c2cc(Cl)c(-c3cc4c(c(C(=O)O)c3)OC(F)(F)O4)cc2S1(=O)=O] | [M + H]⁺ 605.2 | ¹H NMR (400 MHz, DMSO-d₆): δ 13.85 (s, 1H), 7.90 (s, 1H), 7.78 (s, 1H), 7.68 (s, 1H), 7.37-7.23 (m, 3H), 7.10-6.80 (m, 3H), 4.48- 4.26 (m, 1H), 3.71 (s, 1H), 3.33 (s, 1H), 2.71 (s, 3H), 2.00-1.82 (m, 2H), 1.80-1.54 (m, 2H), 1.62 (s, 2H), 1.27-1.14 (m, 3H), 1.06- 0.90 (m, 2H). |

### Ex.155: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-2-ethoxybenzoic acid

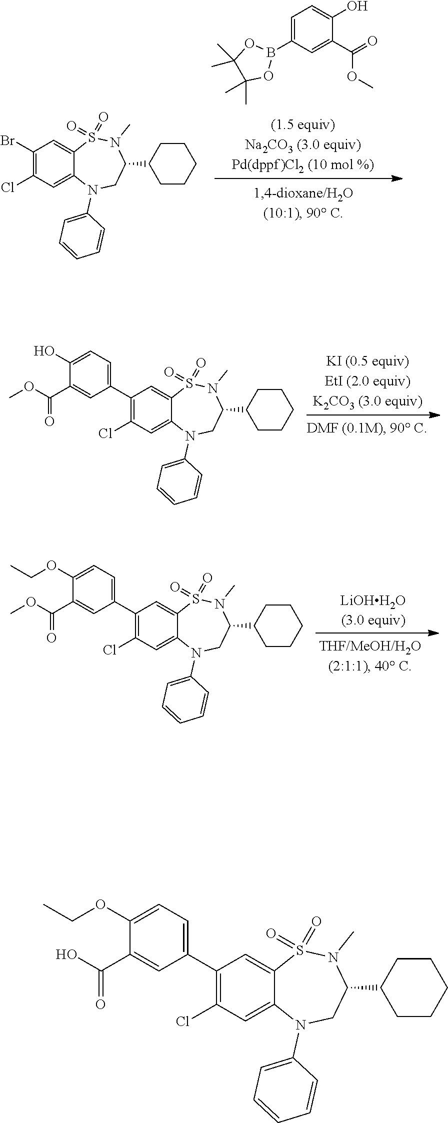

Step 1

To a solution of (R)-8-bromo-7-chloro-3-cyclohexyl-2-methyl-5-phenyl-2,3,4,5-tetrahydrobenzo[f][1,2,5]thiadiazepine 1,1-dioxide (900 mg, 1.9 mmol, 1.0 eq) in 1,4-dioxane (15 mL) and H₂O (1.5 mL) was added methyl 2-hydroxy-5-(4,4,5,5-tetramethyl-1,3,2-dioxaborolan-2-yl)benzoate (776 mg, 2.8 mmol, 1.5 eq), Na₂CO₃ (600 mg, 5.6 mmol, 3.0 eq) and Pd(dppf)C₁₂ (135 mg, 0.2 mmol, 0.1 eq). The mixture was stirred at 90° C. overnight. The mixture was then diluted with water (20 mL) and extracted three times with EtOAc (20 mL each). The combined organic layer was washed twice with water (20 mL each), dried over Na₂SO₄ and concentrated to give a residue, which was purified by silica gel column chromatography (Petroleum ether/EtOAc=20:1) to give methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-hydroxybenzoate (520 mg, 50% yield) as yellow oil. ESI MS m/z=555.0 [M+H]⁺.

Step 2

To a solution of methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-hydroxybenzoate (80 mg, 0.1 mmol, 1.0 eq) in DMF (1.0 mL) was added iodoethane (45 mg, 0.3 mmol, 2.0 eq), KI (12 mg, 0.1 mmol, 0.5 eq) and K₂CO₃ (60 mg, 0.4 mmol, 3.0 eq). The mixture was stirred at 90° C. overnight. The mixture was diluted with water (10 mL) and extracted three times with EtOAc (10 mL each). The combined organic layer was washed twice with water (10 mL each), dried over Na₂SO₄ and concentrated to give a residue, which was purified by silica gel column chromatography (Petroleum ether/EtOAc=50:1) to give methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-ethoxybenzoate (59 mg, 70% yield) as yellow oil. ESI MS m/z=583.0 [M+H]⁺.

Step 3

To a solution of methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-ethoxybenzoate (59 mg, 0.1 mmol, 1.0 eq) in THE (2.0 mL) and MeOH (1.0 mL) was added a solution of LiOH H₂O (13 mg, 0.3 mmol, 3.0 eq) in H₂O (1.0 mL). The reaction mixture was stirred at 40° C. for 4 h. The mixture was then concentrated in vacuo to remove solvent. The residue was adjusted pH to 3-4 with 0.5 N HCl. The resulting precipitate was washed twice with cold water (10 mL) and dried under vacuum to give (R)-5-(7-chloro-3-cyclohexyl-2-methyl-1,1-dioxido-5-phenyl-2,3,4,5-tetrahydrobenzo[f][1,2,5]thiadiazepin-8-yl)-2-ethoxybenzoic acid, Ex. 155 (18 mg, 31% yield) as white solid. ESI MS m/z=569.1 [M+H]⁺. ¹H NMR (300 MHz, DMSO-d₆): δ 8.20 (s, 1H), 7.78-7.50 (m, 3H), 7.40-7.10 (m, 4H), 7.09-6.82 (m, 3H), 4.45-4.28 (m, 1H), 4.15 (q, J=6.9 Hz, 2H), 3.80-3.50 (m, 1H), 3.30-3.20 (m, 1H), 2.67 (s, 3H), 2.08-1.80 (m, 2H), 1.80-1.45 (m, 4H), 1.36 (t, J=6.9 Hz, 3H), 1.30-1.10 (m, 3H), 1.10-0.70 (m, 2H).

The following compounds were prepared using a procedure analogous to that used for Ex.155:

| Example | Structure | MS | NMR |
|---|---|---|---|
| 155 | 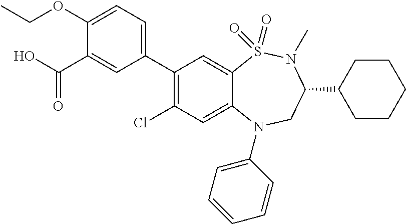 [SMILES: CCOc1ccc(-c2cc3c(cc2Cl)N(c2ccccc2)C[C@@H](C2CCCCC2)N(C)S3(=O)=O)cc1C(=O)O] | [M + H]⁺ 569.1 | ¹H NMR (300 MHz, DMSO-d₆): δ 8.20 (s, 1H), 7.78-7.50 (m, 3H), 7.40-7.10 (m, 4H), 7.09-6.82 (m, 3H), 4.45-4.28 (m, 1H), 4.15 (q, J = 6.9 Hz, 2H), 3.80-3.50 (m, 1H), 3.30-3.20 (m, 1H), 2.67 (s, 3H), 2.08-1.80 (m, 2H), 1.80- 1.45 (m, 4H), 1.36 (t, J = 6.9 Hz, 3H), 1.30-1.10 (m, 3H), 1.10- 0.70 (m, 2H). |

### Ex. 156: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-2-hydroxybenzoic acid

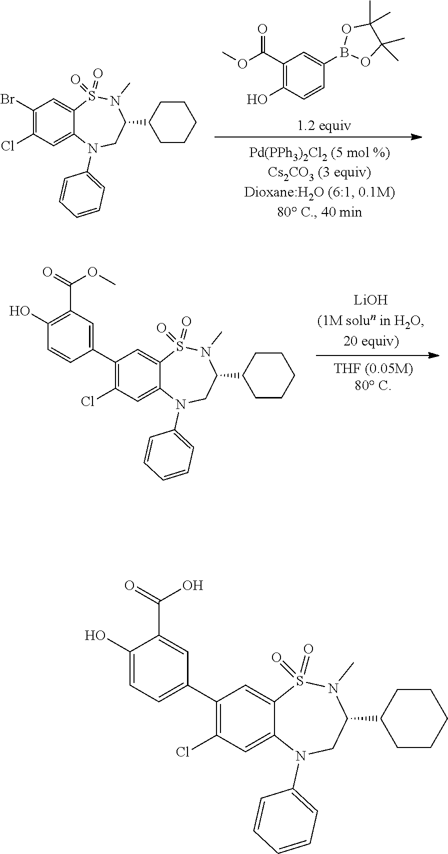

Step 1

In a 4 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (30.0 mg), methyl 2-hydroxy-5-(4,4,5,5-tetramethyl-1,3,2-dioxaborolan-2-yl)benzoate (20.7 mg, 1.2 equiv.), bis(triphenylphosphine)palladium(II) chloride (2.2 mg, 0.05 equiv.) and cesium carbonate (60.6 mg, 3.0 equiv.) were combined neat under nitrogen atmosphere, followed by addition of dioxane (0.53 mL) and water (0.09 mL). The reaction mixture was heated at 80° C. for 40 minutes in a heating block to afford methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-hydroxybenzoate LC-MS. ESI MS m/z=555.4 [M+H]⁺.

Step 2

To the reaction mixture from Step 1, THE (1.25 mL) and LiOH (1M solⁿ in H₂O, 1.24 mL, 20.0 equiv.) were added and the resulting mixture was stirred overnight at 80° C. Reaction progress was monitored by LC-MS. The reaction mixture was quenched with 1N HCl, extracted with ethyl acetate (x3). The combined organic layers were washed with water and brine and dried over sodium sulfate and concentrated. The crude residue was re-dissolved in 2.0 mL of dimethyl sulfoxide, passed through a 0.45 μm syringe filter, and purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-hydroxybenzoic acid, Ex. 156 (9.5 mg, 28% yield). ESI MS m/z=541.3 [M+H]⁺.

| Example | Structure | MS | NMR |
|---|---|---|---|
| 156 | 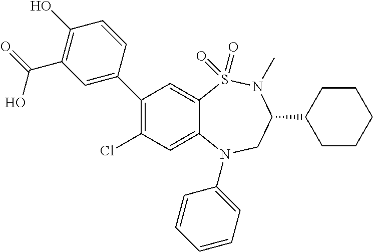 [SMILES: CN1[C@H](C2CCCCC2)CN(c2ccccc2)c2cc(Cl)c(-c3ccc(O)c(C(=O)O)c3)cc2S1(=O)=O] | [M + H]⁺ 541.3 | ¹H NMR (400 MHz, DMSO-d6) δ 11.54 (br, 1H), 7.87 (d, J = 2.4 Hz, 1H), 7.72 (s, 1H), 7.65 (dd, J = 8.6, 2.4 Hz, 1H), 7.35-7.23 (m, 3H), 7.06 (d, J = 8.6 Hz, 1H), 7.03- 6.88 (m, 3H), 4.36 (d, J = 16.1 Hz, 1H), 3.80-3.52 (m, 1H), 2.68 (s, 3H), 2.03-1.52 (m, 6H), 1.33- 0.86 (m, 5H). |

The following compounds were prepared using a procedure analogous to that described for Ex. 156:

| Example | Structure | MS |
|---|---|---|
| 157 | 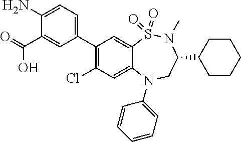 [SMILES: CN1[C@H](C2CCCCC2)CN(c2ccccc2)c2cc(Cl)c(-c3ccc(N)c(C(=O)O)c3)cc2S1(=O)=O] | [M + H]⁺ 540.4 |
| 158 | 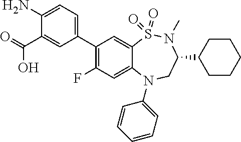 [SMILES: CN1[C@H](C2CCCCC2)CN(c2ccccc2)c2cc(F)c(-c3ccc(N)c(C(=O)O)c3)cc2S1(=O)=O] | [M + H]⁺ 524.1 |
| 159 | 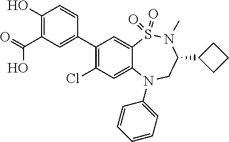 [SMILES: CN1[C@H](C2CCC2)CN(c2ccccc2)c2cc(Cl)c(-c3ccc(O)c(C(=O)O)c3)cc2S1(=O)=O] | [M + H]⁺ 513.1 |

### Ex.160: Synthesis of (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoic acid

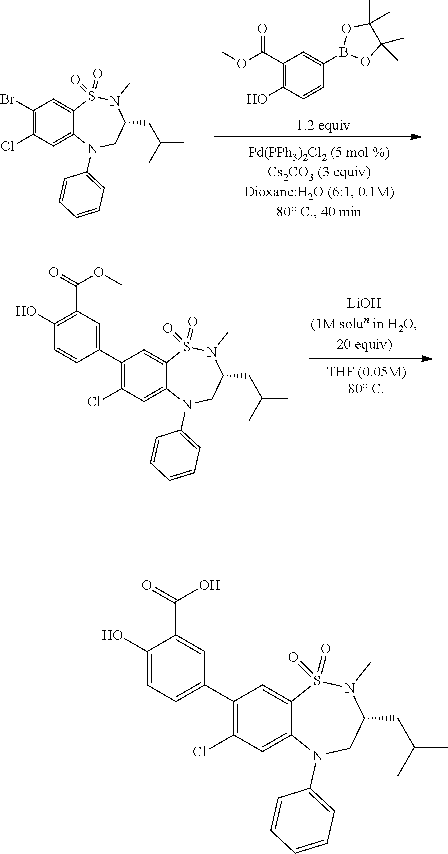

Step 1

In a 20 mL vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (200.0 mg), methyl 2-hydroxy-5-(4,4,5,5-tetramethyl-1,3,2-dioxaborolan-2-yl)benzoate (146.0 mg, 1.2 equiv.), bis(triphenylphosphine)palladium(II) chloride (15.33 mg, 0.05 equiv.) and cesium carbonate (427 mg, 3.0 equiv.) were combined neat under nitrogen atmosphere, followed by addition of dioxane (3.8 ml) and water (0.6 ml). The reaction mixture was heated at 80° C. for 40 minutes in a heating block. The reaction mixture was then quenched with formic acid (0.335 ml) and concentrated. The crude was purified through reversed phase chromatography to afford (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoate (200 mg, 87% yield). ESI MS m/z=529.4 [M+H]⁺.

Step 2

In a 4 mL vial equipped with a stir bar, (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoate (25.0 mg, 1.0 equiv.) was dissolved in tetrahydrofuran (0.945 mL). Lithium hydroxide (1M solution in water, 0.945 mL, 20.0 equiv.) was added subsequently, and the reaction mixture was stirred overnight at 80° C. in a heating block. Reaction progress was monitored by LC-MS. Upon completion, the reaction mixture was quenched with 1N HCl and extracted three times with ethyl acetate. The combined organic layers were washed with water and brine and dried over sodium sulfate and concentrated. The crude residue was re-dissolved in 2.0 mL of dimethyl sulfoxide, passed through a 0.45 μm syringe filter, and purified by RPHPLC to afford (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoic acid, Ex.160 (4.7 mg, 19% yield). ESI MS m/z=515.4 [M+H]⁺.

| Example | Structure | MS | NMR |
|---|---|---|---|
| 160 | 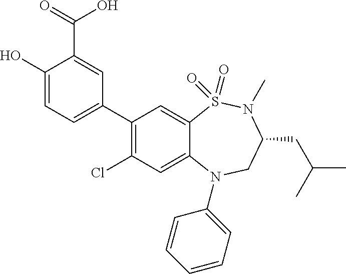 [SMILES: CC(C)C[C@@H]1CN(c2ccccc2)c2cc(Cl)c(-c3ccc(O)c(C(=O)O)c3)cc2S(=O)(=O)N1C] | [M + H]⁺ 515.4 | ¹H NMR (500 MHz, DMSO-d6) δ 7.87 (d, J = 2.5 Hz, 1H), 7.76 (s, 1H), 7.61 (d, J = 8.8 Hz, 1H), 7.44 (s, 1H), 7.25 (dd, J = 8.5, 7.2 Hz, 2H), 7.01 (d, J = 8.5, 1H), 6.92 − 6.83 (m, 3H), 4.16 − 4.01 (m, 1H), 3.96 − 3.82 (m, 1H), 2.58 (s, 3H), 1.77 − 1.63 (m, 1H), 1.61 − 1.48 (m, 1H), 1.40 − 1.31 (m, 1H), 0.93 (d, J = 6.6 Hz, 3H), 0.89 (d, J = 6.5 Hz, 3H). |

The following compounds were prepared using a procedure analogous to that described for Ex. 160:

| Example | Structure | MS | NMR |
|---|---|---|---|
| 161 | 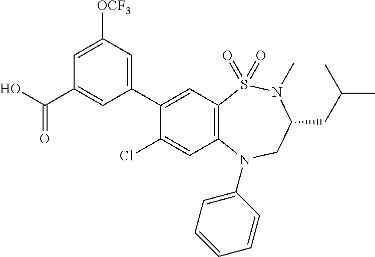 [SMILES: CC(C)C[C@@H]1CN(c2ccccc2)c2cc(Cl)c(-c3cc(OC(F)(F)F)cc(C(=O)O)c3)cc2S(=O)(=O)N1C] | [M + H]⁺ 583.3 | ¹H NMR (400 MHz, DMSO-d6) δ 13.70 (br, 1H), 8.07 − 7.02 (m, 1H), 7.91 − 7.81 (m, 3H), 7.44 (s, 1H), 7.28 (t, J = 7.9 Hz, 2H), 6.96 − 6.87 (m, 3H), 4.10 (d, J = 16.1 Hz, 1H), 3.94 − 3.81 (,, 1H), 3.56 − 3.40 (m, 1H), 2.62 (s, 3H), 1.76 − 1.63 (m, 1H), 1.62 − 1.47 (m, 1H), 1.43 − 1.27 (m 1H), 0.92 (d, J = 6.6 Hz, 3H), 0.89 (d, J = 6.5 Hz, 3H). |
| 162 | 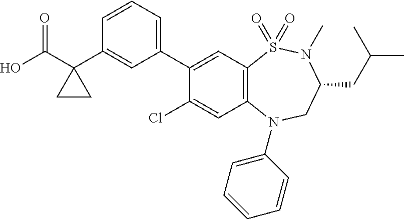 [SMILES: CC(C)C[C@@H]1CN(c2ccccc2)c2cc(Cl)c(-c3cccc(C4(C(=O)O)CC4)c3)cc2S(=O)(=O)N1C] | [M + H]⁺ 539.5 | ¹H NMR (400 MHz, DMSO-d6) δ 12.45 (br, 1H), 7.77 (s, 1H), 7.48 − 7.35 (m, 5H), 7.31 − 7.22 (m, 2H), 6.93 − 6.82 (m, 3H), 4.09 (d, J = 16.0 Hz, 1H), 3.98 − 3.82 (m, 1H), 3.51 − 3.41 (m, 1H), 2.59 (s, 3H), 1.76 − 1.64 (m, 1H), 1.61 − 1.49 (m, 1H), 1.48 − 1.30 (m, 3H), 1.20 − 1.12 (m, 2H), 0.93 (d, J = 6.6 Hz, 3H), 0.89 (d, J = 6.5 Hz, 3H). |

### Ex.163: Synthesis of (R)-5-(7-chloro-3-isobutyl-2-methyl-1,1-dioxido-5-phenyl-2,3,4,5-tetrahydrobenzo[f][1,2,5]thiadiazepin-8-yl)-2-(trifluoromethoxy)benzoic acid

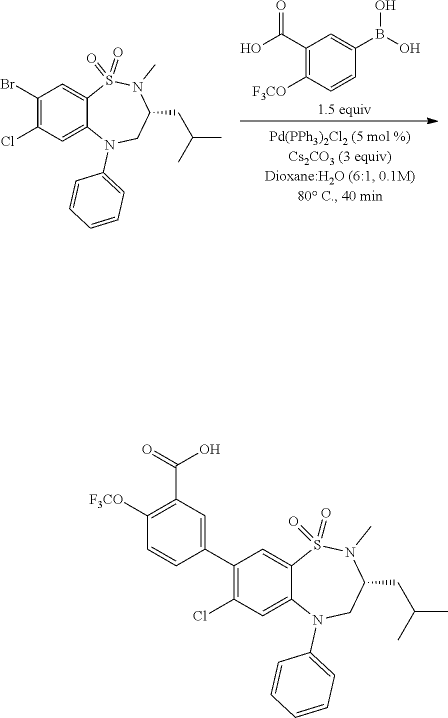 [SMILES: CC(C)C[C@@H]1CN(c2ccccc2)c2cc(Cl)c(Br)cc2S(=O)(=O)N1C ; O=C(O)c1cc(B(O)O)ccc1OC(F)(F)F ; CC(C)C[C@@H]1CN(c2ccccc2)c2cc(Cl)c(-c3ccc(OC(F)(F)F)c(C(=O)O)c3)cc2S(=O)(=O)N1C]

In a 20 mL vial equipped with a stir bar, (R)-8-bromo-7-chloro-3-isobutyl-2-methyl-5-phenyl-2,3,4,5-tetrahydrobenzo[f][1,2,5]thiadiazepine 1,1-dioxide (20.0 mg, 1.0 equiv), 5-borono-2-(trifluoromethoxy)benzoic acid (16.4 mg, 1.5 equiv), bis(triphenylphosphine)palladium(II) chloride (1.5 mg, 0.05 equiv) and cesium carbonate (42.7 mg, 3.0 equiv) were combined neat under nitrogen atmosphere, followed by addition of dioxane (0.38 mL) and water (0.057 mL). Reaction mixture was heated at 80° C. for 40 minutes in a heating block. After completion, the reaction mixture was quenched with formic acid (0.0335 ml) and concentrated under vacuum. The crude residue was re-dissolved in 2.0 mL of dimethyl sulfoxide, passed through a 0.45 μm syringe filter, and purified by RPHPLC to afford (R)-5-(7-chloro-3-isobutyl-2-methyl-1,1-dioxido-5-phenyl-2,3,4,5-tetrahydrobenzo[f][1,2,5]thiadiazepin-8-yl)-2-(trifluoromethoxy)benzoic acid, Ex.163 (13.0 mg, 51% yield). ESI MS m/z=583.3 [M+H]⁺.

| Example | Structure | MS | NMR |
|---|---|---|---|
| 163 | 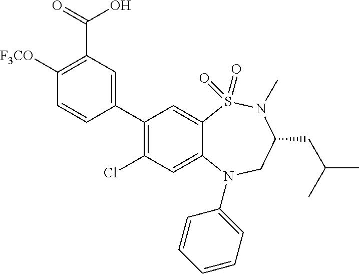 [SMILES: CC(C)C[C@@H]1CN(c2ccccc2)c2cc(Cl)c(-c3ccc(OC(F)(F)F)c(C(=O)O)c3)cc2S(=O)(=O)N1C] | [M + H]⁺ 583.3 | ¹H NMR (500 MHz, DMSO-d6) δ 13.60 (br, 1H), 7.98 (s, 1H), 7.85 − 7.78 (m, 2H), 7.58 (d, J = 8.4 Hz, 1H), 7.44 (s, 1H), 7.28 (t, J = 7.8 Hz, 2H), 6.91 (dd, J = 8.4, 5.0 Hz, 3H), 4.10 (d, J = 16.1 Hz, 1H), 3.95 − 3.81 (m, 1H), 3.57 − 3.41 (m, 1H), 2.61 (s, 3H), 1.76 − 1.63 (m, 1H), 1.61 − 1.50 (m, 1H), 1.41 − 1.30 (m, 1H), 0.92 (d, J = 6.6 Hz, 3H), 0.89 (d, J = 6.4 Hz, 3H). |

The following compounds were prepared using a procedure analogous to that described or Ex. 163:

| Example | Structure | MS | NMR |
|---|---|---|---|
| 164 | 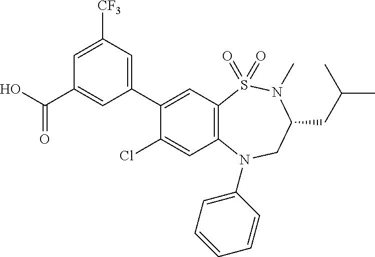 [SMILES: CC(C)C[C@@H]1CN(c2ccccc2)c2cc(Cl)c(-c3cc(C(=O)O)cc(C(F)(F)F)c3)cc2S(=O)(=O)N1C] | [M + H]⁺ 567.3 | ¹H NMR (400 MHz, DMSO-d6) δ 8.32 − 8.21 (m, 2H), 8.16 (s, 1H), 7.87 (s, 1H), 7.45 (s, 1H), 7.28 (t, J = 7.9 Hz, 2H), 6.91 (dd, J = 8.3, 5.2 Hz, 3H), 4.11 (d, J = 16.0 Hz, 1H), 3.96 − 3.80 (m, 1H), 2.62 (s, 3H), 1.77 − 1.64 (m, 1H), 1.63 − 1.51 (m, 1H), 1.42 − 1.30 (m 1H), 0.93 (d, J = 6.6 Hz, 3H), 0.89 (d, J = 6.5 Hz, 3H). |
| 165 | 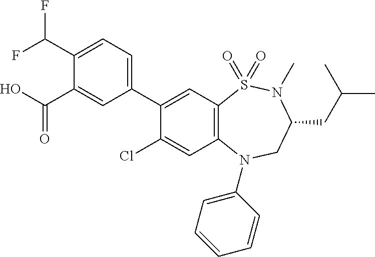 [SMILES: CC(C)C[C@@H]1CN(c2ccccc2)c2cc(Cl)c(-c3ccc(C(F)F)c(C(=O)O)c3)cc2S(=O)(=O)N1C] | [M + H]⁺ 549.4 | |
| 166 | 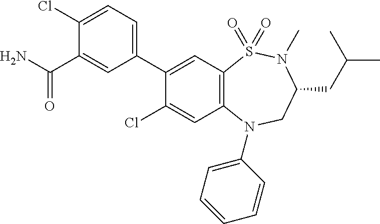 [SMILES: CC(C)C[C@@H]1CN(c2ccccc2)c2cc(Cl)c(-c3ccc(Cl)c(C(N)=O)c3)cc2S(=O)(=O)N1C] | [M + H]⁺ 532.3 | |
| 167 | 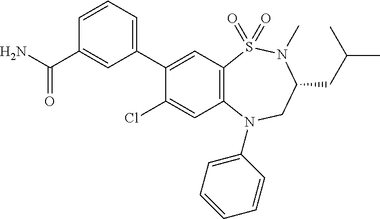 [SMILES: CC(C)C[C@@H]1CN(c2ccccc2)c2cc(Cl)c(-c3cccc(C(N)=O)c3)cc2S(=O)(=O)N1C] | [M + H]⁺ 498.3 | |
| 168 | 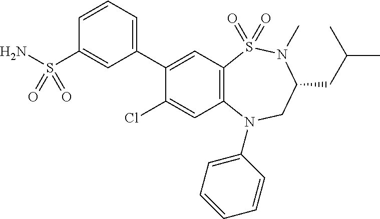 [SMILES: CC(C)C[C@@H]1CN(c2ccccc2)c2cc(Cl)c(-c3cccc(S(N)(=O)=O)c3)cc2S(=O)(=O)N1C] | [M + H]⁺ 534.3 | |
| 169 | 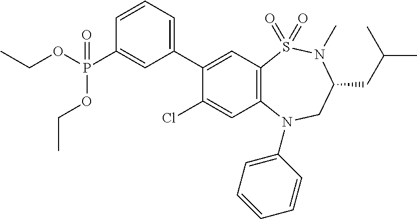 [SMILES: CCOP(=O)(OCC)c1cccc(-c2cc3c(cc2Cl)N(c2ccccc2)C[C@@H](CC(C)C)N(C)S3(=O)=O)c1] | [M + H]⁺ 591.1 | |
| 170 | 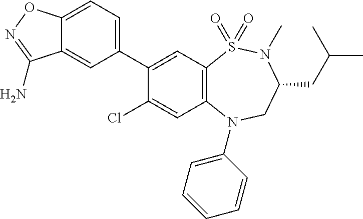 [SMILES: CC(C)C[C@@H]1CN(c2ccccc2)c2cc(Cl)c(-c3ccc4onc(N)c4c3)cc2S(=O)(=O)N1C] | [M + H]⁺ 511.4 | |
| 171 | 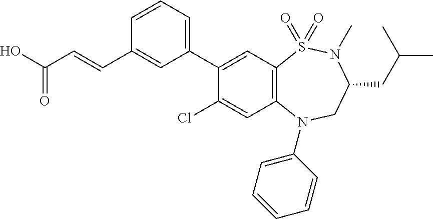 [SMILES: CC(C)C[C@@H]1CN(c2ccccc2)c2cc(Cl)c(-c3cccc(/C=C/C(=O)O)c3)cc2S(=O)(=O)N1C] | [M + H]⁺ 525.3 | |
| 172 | 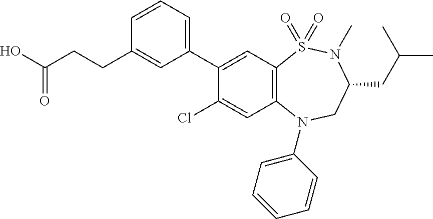 [SMILES: CC(C)C[C@@H]1CN(c2ccccc2)c2cc(Cl)c(-c3cccc(CCC(=O)O)c3)cc2S(=O)(=O)N1C] | [M + H]⁺ 527.4 | |
| 173 | 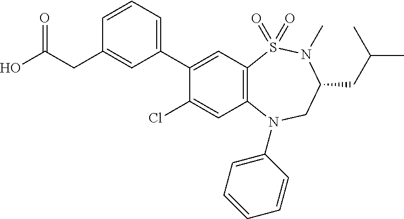 [SMILES: CC(C)C[C@@H]1CN(c2ccccc2)c2cc(Cl)c(-c3cccc(CC(=O)O)c3)cc2S(=O)(=O)N1C] | [M + H]⁺ 513.4 | |
| 174 | 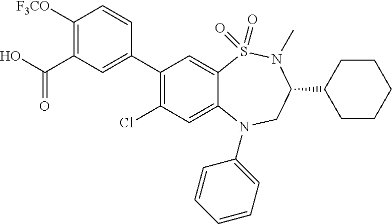 [SMILES: CN1[C@H](C2CCCCC2)CN(c2ccccc2)c2cc(Cl)c(-c3ccc(OC(F)(F)F)c(C(=O)O)c3)cc2S1(=O)=O] | [M + H]⁺ 609.2 | |
| 175 | 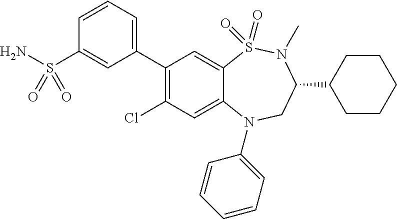 [SMILES: CN1[C@H](C2CCCCC2)CN(c2ccccc2)c2cc(Cl)c(-c3cccc(S(N)(=O)=O)c3)cc2S1(=O)=O] | [M + H]⁺ 560.4 | |
| 176 | 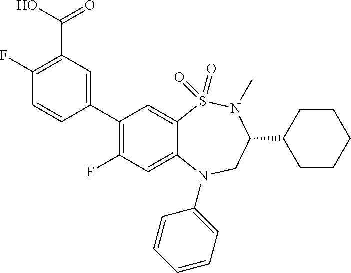 [SMILES: CN1[C@H](C2CCCCC2)CN(c2ccccc2)c2cc(F)c(-c3ccc(F)c(C(=O)O)c3)cc2S1(=O)=O] | [M + H]⁺ 527.2 | |
| 177 | 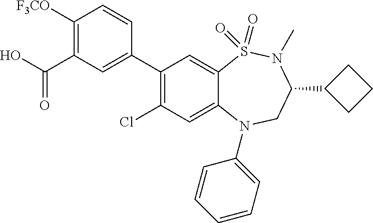 [SMILES: CN1[C@H](C2CCC2)CN(c2ccccc2)c2cc(Cl)c(-c3ccc(OC(F)(F)F)c(C(=O)O)c3)cc2S1(=O)=O] | [M + H]⁺ 581.2 | |
| 178 | 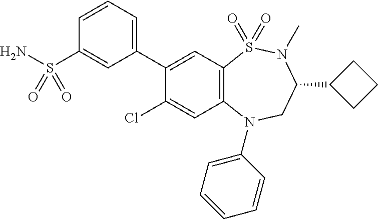 [SMILES: CN1[C@H](C2CCC2)CN(c2ccccc2)c2cc(Cl)c(-c3cccc(S(N)(=O)=O)c3)cc2S1(=O)=O] | [M + H]⁺ 532.3 | |

### Ex.179: (R)-5-(7-chloro-3-isobutyl-2-methyl-1,1-dioxido-5-phenyl-2,3,4,5-tetrahydrobenzo[f][1,2,5]thiadiazepin-8-yl)-2-fluoro-3-methoxybenzoic acid

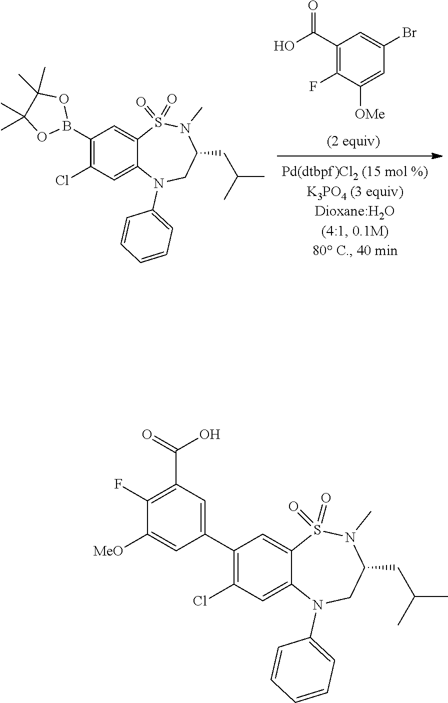 [SMILES: CC(C)C[C@@H]1CN(c2ccccc2)c2cc(Cl)c(B3OC(C)(C)C(C)(C)O3)cc2S(=O)(=O)N1C ; COc1cc(Br)cc(C(=O)O)c1F ; COc1cc(-c2cc3c(cc2Cl)N(c2ccccc2)C[C@@H](CC(C)C)N(C)S3(=O)=O)cc(C(=O)O)c1F]

In a 4 mL vial equipped with a stir bar, (R)-7-chloro-3-isobutyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (30.0 mg, 1.0 equiv.), 5-bromo-2-fluoro-3-methoxybenzoic acid (29.6 mg, 2.0 equiv., CAS #: 1782260-95-0), [1,1′-bis(di-tert-butylphosphino) ferrocene]dichloropalladium(II) (5.8 mg, 0.15 equiv., CAS #: 95408-45-0) and potassium phosphate tribasic (37.8 mg, 3.0 equiv., CAS #: 7778-53-2) were combined neat under nitrogen atmosphere and followed by addition of 1,4-dioxane (0.48 mL) and water (0.12 mL). The reaction mixture was heated at 80° C. for 40 min in a heating block. Upon cooling to room temperature, the reaction mixture was quenched with formic acid (0.2 mL) and concentrated under vacuum. The crude residue was re-dissolved in 2.0 mL of dimethyl sulfoxide, passed through a 0.45 μm syringe filter, and purified by RPHPLC to afford (R)-5-(7-chloro-3-isobutyl-2-methyl-1,1-dioxido-5-phenyl-2,3,4,5-tetrahydrobenzo[f][1,2,5]thiadiazepin-8-yl)-2-fluoro-3-methoxybenzoic acid, Ex.179. ESI MS m/z=547.4 [M+H]⁺.

| Example | Structure | MS | NMR |
|---|---|---|---|
| 179 | 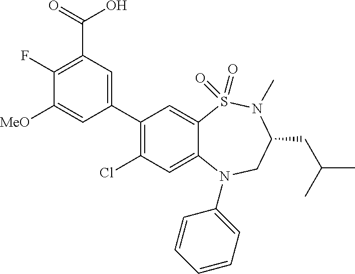 [SMILES: COc1cc(-c2cc3c(cc2Cl)N(c2ccccc2)C[C@@H](CC(C)C)N(C)S3(=O)=O)cc(C(=O)O)c1F] | [M + H]⁺ 547.4 | ¹H NMR (500 MHz, DMSO-d6) δ 13.46 (br, 1H), 7.81 (s, 1H), 7.51 − 7.41 (m, 2H), 7.39 − 7.34 (m, 1H), 7.27 (t, J = 7.7 Hz, 2H), 6.93 − 6.85 (m, 3H), 4.10 (d, J = 16.0 Hz, 1H), 3.91 (s, 3H), 3.90 − 3.83 (m, 1H), 3.53 − 3.39 (m, 1H), 2.60 (s, 3H), 1.76 − 1.63 (m, 1H), 1.63 − 1.49 (m, 1H), 1.42 − 1.31(m, 1H), 0.93 (d, J = 6.6 Hz, 3H), 0.89 (d, J = 6.4 Hz, 3H). |

The following compounds were prepared using a procedure analogous to that described for Ex. 179:

| Example | Structure | MS |
|---|---|---|
| 180 | 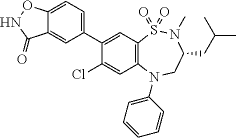 [SMILES: CC(C)C[C@@H]1CN(c2ccccc2)c2cc(Cl)c(-c3ccc4o[nH]c(=O)c4c3)cc2S(=O)(=O)N1C] | [M + H]⁺ 512.3 |
| 181 | 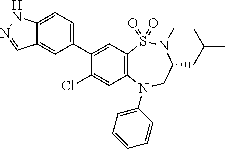 [SMILES: CC(C)C[C@@H]1CN(c2ccccc2)c2cc(Cl)c(-c3ccc4[nH]ncc4c3)cc2S(=O)(=O)N1C] | [M + H]⁺ 495.4 |
| 182 | 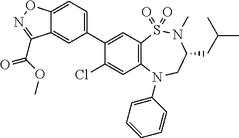 [SMILES: COC(=O)c1noc2ccc(-c3cc4c(cc3Cl)N(c3ccccc3)C[C@@H](CC(C)C)N(C)S4(=O)=O)cc12] | [M + H]⁺ 554.3 |
| 183 | 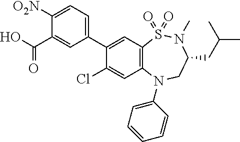 [SMILES: CC(C)C[C@@H]1CN(c2ccccc2)c2cc(Cl)c(-c3ccc([N+](=O)[O-])c(C(=O)O)c3)cc2S(=O)(=O)N1C] | [M + H]⁺ 544.3 |
| 184 | 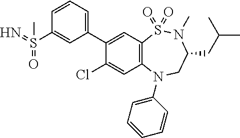 [SMILES: CC(C)C[C@@H]1CN(c2ccccc2)c2cc(Cl)c(-c3cccc(S(C)(=N)=O)c3)cc2S(=O)(=O)N1C] | [M + H]⁺ 532.3 |
| 185 | 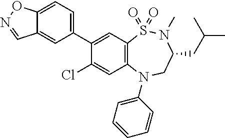 [SMILES: CC(C)C[C@@H]1CN(c2ccccc2)c2cc(Cl)c(-c3ccc4oncc4c3)cc2S(=O)(=O)N1C] | [M + H]⁺ 496.4 |
| 186 | 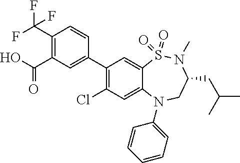 [SMILES: CC(C)C[C@@H]1CN(c2ccccc2)c2cc(Cl)c(-c3ccc(C(F)(F)F)c(C(=O)O)c3)cc2S(=O)(=O)N1C] | [M + H]⁺ 567.3 |
| 187 | 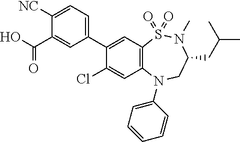 [SMILES: CC(C)C[C@@H]1CN(c2ccccc2)c2cc(Cl)c(-c3ccc(C#N)c(C(=O)O)c3)cc2S(=O)(=O)N1C] | [M + H]⁺ 524.3 |
| 188 | 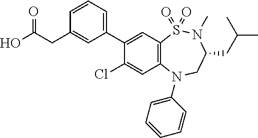 [SMILES: CC(C)C[C@@H]1CN(c2ccccc2)c2cc(Cl)c(-c3cccc(CC(=O)O)c3)cc2S(=O)(=O)N1C] | [M + H]⁺ 513.3 |

### Ex.189: (R)-5-(7-chloro-3-cyclohexyl-2-methyl-1,1-dioxido-5-phenyl-2,3,4,5-tetrahydrobenzo[f][1,2,5]thiadiazepin-8-yl)-2-nitrobenzoic acid

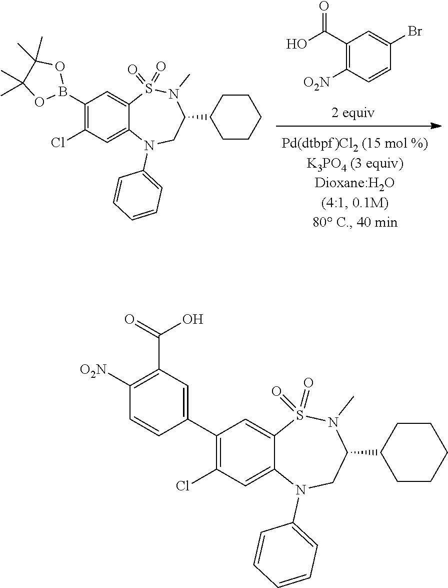 [SMILES: CN1[C@H](C2CCCCC2)CN(c2ccccc2)c2cc(Cl)c(B3OC(C)(C)C(C)(C)O3)cc2S1(=O)=O ; O=C(O)c1cc(Br)ccc1[N+](=O)[O-] ; CN1[C@H](C2CCCCC2)CN(c2ccccc2)c2cc(Cl)c(-c3ccc([N+](=O)[O-])c(C(=O)O)c3)cc2S1(=O)=O]

In a 4 mL vial equipped with a stir bar, ((R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (40.0 mg, 1.0 equiv.), 5-bromo-2-nitrobenzoic acid (37.1 mg, 2.0 equiv., CAS #: 6950-43-2), [1,1′-bis(di-tert-butylphosphino) ferrocene]dichloropalladium(II) (7.4 mg, 0.15 equiv., CAS #: 95408-45-0) and potassium phosphate tribasic (48.0 mg, 3.0 equiv., CAS #: 7778-53-2) were combined neat under nitrogen atmosphere and followed by addition of 1,4-dioxane (0.60 mL) and water (0.15 mL). The reaction mixture was heated at 80° C. for 40 min in a heating block. Upon cooling to room temperature, the reaction mixture was quenched with formic acid (0.2 mL) and concentrated under vacuum. The crude residue was re-dissolved in 2.0 mL of dimethyl sulfoxide, passed through a 0.45 μm syringe filter, and purified by RPHPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-nitrobenzoic acid, Ex.189 (13.6 mg, 32% yield). ESI MS m/z=570.3 [M+H]⁺.

| Example | Structure | MS | NMR |
|---|---|---|---|
| 189 | 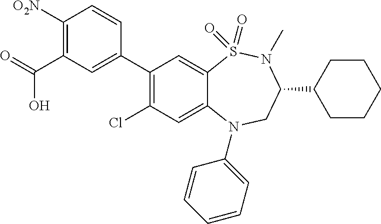 [SMILES: CN1[C@H](C2CCCCC2)CN(c2ccccc2)c2cc(Cl)c(-c3ccc([N+](=O)[O-])c(C(=O)O)c3)cc2S1(=O)=O] | [M + H]⁺ 570.3 | ¹H NMR (500 MHz, DMSO-d6) δ 14.04 (br, 1H), 8.07 (d, J = 8.4 Hz, 1H), 7.93 (s, 1H), 7.88 (d, J = 8.4 Hz, 1H), 7.82 (s, 1H), 7.35 (t, J = 7.7 Hz, 2H), 7.24 (br, 1H), 7.14 − 6.96 (m, 3H), 4.36 (d, J = 16.0 Hz, 1H), 4.01 − 3.57 (s, 1H), 2.73 (s, 3H), 2.00 − 1.84 (m, 2H), 1.76 − 1.67 (m, 2H), 1.66 − 1.54 (m, 2H), 1.30 − 1.07 (m, 3H), 1.04 − 0.89 (m, 2H). |

The following compounds were prepared using a procedure analogous to that described for Ex. 189:

| Example | Structure | MS |
|---|---|---|
| 190 | 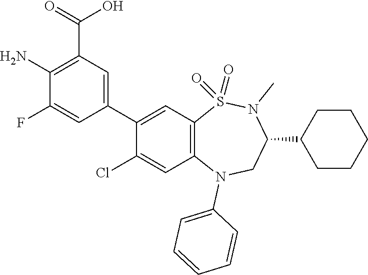 [SMILES: CN1[C@H](C2CCCCC2)CN(c2ccccc2)c2cc(Cl)c(-c3cc(F)c(N)c(C(=O)O)c3)cc2S1(=O)=O] | [M + H]⁺ 558.2 |
| 191 | 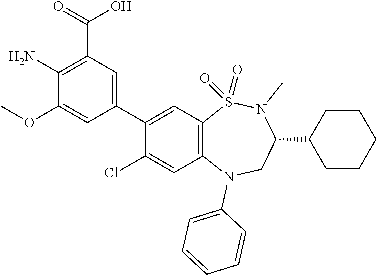 [SMILES: COc1cc(-c2cc3c(cc2Cl)N(c2ccccc2)C[C@@H](C2CCCCC2)N(C)S3(=O)=O)cc(C(=O)O)c1N] | [M + H]⁺ 570.3 |
| 192 | 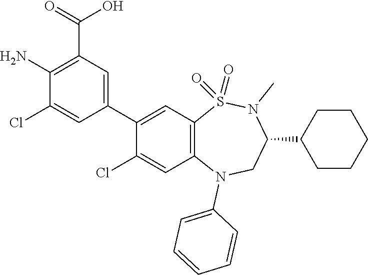 [SMILES: CN1[C@H](C2CCCCC2)CN(c2ccccc2)c2cc(Cl)c(-c3cc(Cl)c(N)c(C(=O)O)c3)cc2S1(=O)=O] | [M + H]⁺ 574.2 |
| 193 | 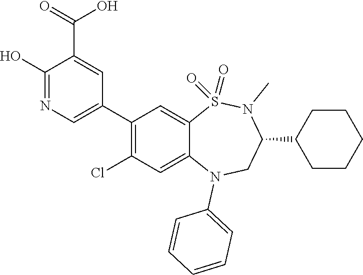 [SMILES: CN1[C@H](C2CCCCC2)CN(c2ccccc2)c2cc(Cl)c(-c3cnc(O)c(C(=O)O)c3)cc2S1(=O)=O] | [M + H]⁺ 542.0 |
| 194 | 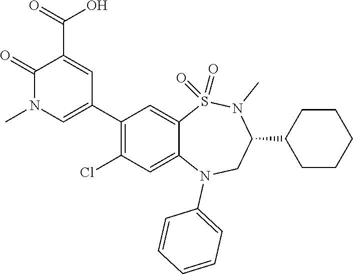 | [M + H]⁺ 1300 |
| 195 | 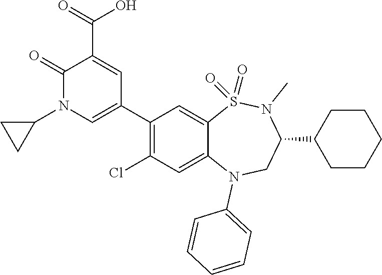 | [M + H]⁺ 560 |
| 196 | 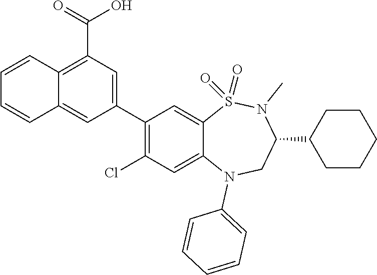 [SMILES: CN1[C@H](C2CCCCC2)CN(c2ccccc2)c2cc(Cl)c(-c3cc(C(=O)O)c4ccccc4c3)cc2S1(=O)=O] | [M + H]⁺ 575.2 |
| 197 | 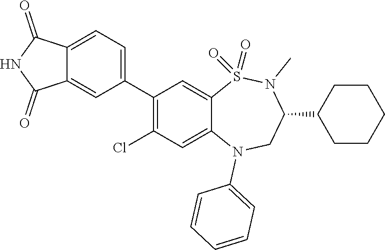 [SMILES: CN1[C@H](C2CCCCC2)CN(c2ccccc2)c2cc(Cl)c(-c3ccc4c(c3)C(=O)NC4=O)cc2S1(=O)=O] | [M + H]⁺ 550.0 |
| 198 | 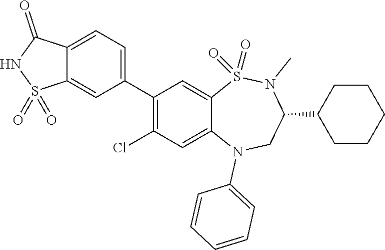 [SMILES: CN1[C@H](C2CCCCC2)CN(c2ccccc2)c2cc(Cl)c(-c3ccc4c(c3)S(=O)(=O)NC4=O)cc2S1(=O)=O] | [M + H]⁺ 586.1 |
| 199 | 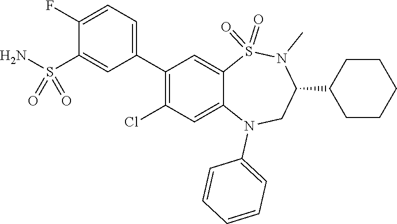 [SMILES: CN1[C@H](C2CCCCC2)CN(c2ccccc2)c2cc(Cl)c(-c3ccc(F)c(S(N)(=O)=O)c3)cc2S1(=O)=O] | [M + H]⁺ 578.4 |
| 200 | 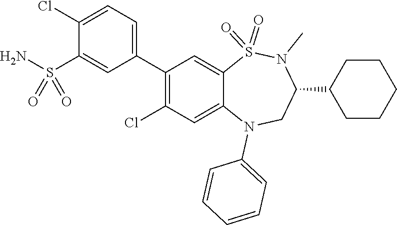 [SMILES: CN1[C@H](C2CCCCC2)CN(c2ccccc2)c2cc(Cl)c(-c3ccc(Cl)c(S(N)(=O)=O)c3)cc2S1(=O)=O] | [M + H]⁺ 594.3 |
| 201 | 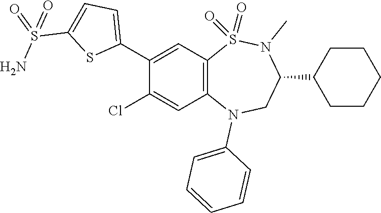 [SMILES: CN1[C@H](C2CCCCC2)CN(c2ccccc2)c2cc(Cl)c(-c3ccc(S(N)(=O)=O)s3)cc2S1(=O)=O] | [M + H]⁺ 566.1 |
| 202 | 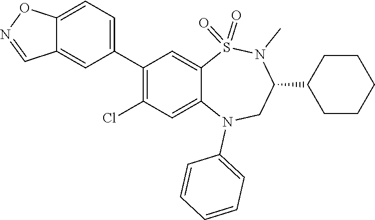 [SMILES: CN1[C@H](C2CCCCC2)CN(c2ccccc2)c2cc(Cl)c(-c3ccc4oncc4c3)cc2S1(=O)=O] | [M + H]⁺ 522.2 |
| 203 | 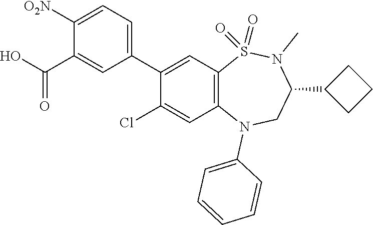 [SMILES: CN1[C@H](C2CCC2)CN(c2ccccc2)c2cc(Cl)c(-c3ccc([N+](=O)[O-])c(C(=O)O)c3)cc2S1(=O)=O] | [M + H]⁺ 542.1 |
| 204 | 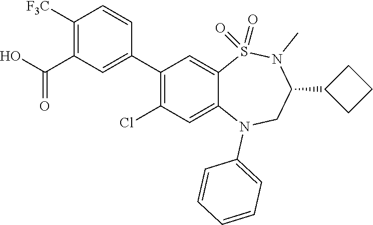 [SMILES: CN1[C@H](C2CCC2)CN(c2ccccc2)c2cc(Cl)c(-c3ccc(C(F)(F)F)c(C(=O)O)c3)cc2S1(=O)=O] | [M + H]⁺ 565.1 |

### Ex.205: (R)-2-(3-(7-chloro-3-isobutyl-2-methyl-1,1-dioxido-5-phenyl-2,3,4,5-tetrahydrobenzo[f][1,2,5]thiadiazepin-8-yl)phenyl)-2-oxoacetic acid

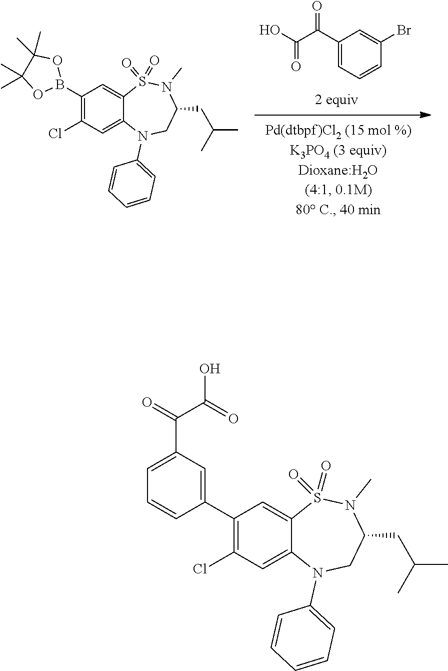 [SMILES: CC(C)C[C@@H]1CN(c2ccccc2)c2cc(Cl)c(B3OC(C)(C)C(C)(C)O3)cc2S(=O)(=O)N1C ; O=C(O)C(=O)c1cccc(Br)c1 ; CC(C)C[C@@H]1CN(c2ccccc2)c2cc(Cl)c(-c3cccc(C(=O)C(=O)O)c3)cc2S(=O)(=O)N1C]

In a 4 mL vial equipped with a stir bar, (R)-7-chloro-3-isobutyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (30.0 mg, 1.0 equiv.), 3-Bromo-α-oxobenzeneacetic acid (27.2 mg, 2.0 equiv., CAS #: 7194-78-7), [1,1′-bis(di-tert-butylphosphino) ferrocene]dichloropalladium(II) (5.8 mg, 0.15 equiv., CAS #: 95408-45-0) and potassium phosphate tribasic (37.8 mg, 3.0 equiv., CAS #: 7778-53-2) were combined neat under nitrogen atmosphere and followed by addition of 1,4-dioxane (0.48 mL) and water (0.12 mL). The reaction mixture was heated at 80° C. for 40 minutes in a heating block. Upon cooling to room temperature, the reaction mixture was quenched with formic acid (0.2 mL) and concentrated under vacuum. The crude residue was purified through normal phase silica gel column chromatography (DCM/MeOH) to afford (R)-2-(3-(7-chloro-3-isobutyl-2-methyl-1,1-dioxido-5-phenyl-2,3,4,5-tetrahydrobenzo[f][1,2,5]thiadiazepin-8-yl)phenyl)-2-oxoacetic acid, Ex.205 (5.0 mg, 16% yield). ESI MS m/z=527.4 [M+H]⁺.

| Example | Structure | MS | NMR |
|---|---|---|---|
| 205 | 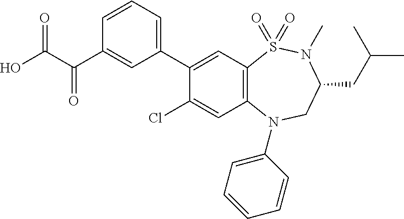 [SMILES: CC(C)C[C@@H]1CN(c2ccccc2)c2cc(Cl)c(-c3cccc(C(=O)C(=O)O)c3)cc2S(=O)(=O)N1C] | [M + H]⁺ 527.4 | ¹H NMR (500 MHz, DMSO) δ 7.92 − 7.85 (m, 2H), 7.77 (s, 1H), 7.73 − 7.68 (m, 1H), 7.61 (t, J = 7.6 Hz, 1H), 7.46 (s, 1H), 7.27 (t, J = 7.9 Hz, 2H), 6.93 − 6.86 (m, 3H), 4.10 (d, J = 16.2 Hz, 1H), 3.95 − 3.83 (m, 1H), 3.56 − 3.40 (m, 1H), 2.61 (s, 3H), 1.74 − 1.68 (m, 1H), 1.59 − 1.49 (m, 1H), 1.40 − 1.31 (m, 1H), 0.92 (d, J = 6.6 Hz, 3H), 0.89 (d, J = 6.4 Hz, 3H). |

The following compounds were prepared using a procedure analogous to that described for Ex.205.

| Example | Structure | MS |
|---|---|---|
| 206 | 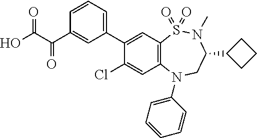 [SMILES: CN1[C@H](C2CCC2)CN(c2ccccc2)c2cc(Cl)c(-c3cccc(C(=O)C(=O)O)c3)cc2S1(=O)=O] | [M + H]⁺ 525.2 |
| 207 | 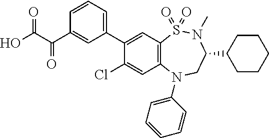 [SMILES: CN1[C@H](C2CCCCC2)CN(c2ccccc2)c2cc(Cl)c(-c3cccc(C(=O)C(=O)O)c3)cc2S1(=O)=O] | [M + H]⁺ 553.1 |

### Ex.208: Synthesis of(R)-6-(7-chloro-3-cyclohexyl-2-methyl-1,1-dioxido-5-phenyl-2,3,4,5-tetrahydrobenzo[f][1,2,5]thiadiazepin-8-yl)-1H-indazole-4-carboxylic acid

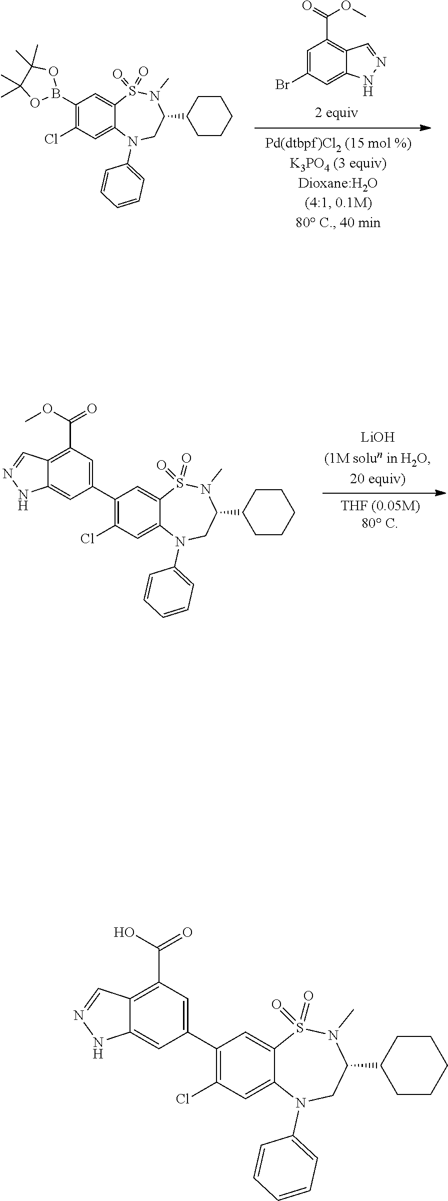 [SMILES: CN1[C@H](C2CCCCC2)CN(c2ccccc2)c2cc(Cl)c(B3OC(C)(C)C(C)(C)O3)cc2S1(=O)=O ; COC(=O)c1cc(Br)cc2[nH]ncc12 ; COC(=O)c1cc(-c2cc3c(cc2Cl)N(c2ccccc2)C[C@@H](C2CCCCC2)N(C)S3(=O)=O)cc2[nH]ncc12 ; CN1[C@H](C2CCCCC2)CN(c2ccccc2)c2cc(Cl)c(-c3cc(C(=O)O)c4cn[nH]c4c3)cc2S1(=O)=O]

Step 1

In a 4 mL vial equipped with a stir bar, (R)-7-chloro-3-cyclohexyl-2-methyl-5-phenyl-8-(4,4,5,5-tetramethyl-1,3,2-dioxaborolan-2-yl)-2,3,4,5-tetrahydrobenzo[f][1,2,5]thiadiazepine 1,1-dioxide (40.0 mg, 1.0 equiv.), methyl 6-bromo-1H-indazole-4-carboxylate (38.4 mg, 2.0 equiv., CAS #: 885518-49-0), [1,1′-bis(di-tert-butylphosphino) ferrocene]dichloropalladium(II) (7.4 mg, 0.15 equiv., CAS #: 95408-45-0) and potassium phosphate tribasic (48.0 mg, 3.0 equiv., CAS #: 7778-53-2) were combined neat under nitrogen atmosphere and followed by addition of 1,4-dioxane (0.58 mL) and water (0.15 mL). The reaction mixture was heated at 80° C. for 40 min in a heating block to afford methyl (R)-6-(7-chloro-3-cyclohexyl-2-methyl-1,1-dioxido-5-phenyl-2,3,4,5-tetrahydrobenzo[f][1,2,5]thiadiazepin-8-yl)-1H-indazole-4-carboxylate. ESI MS m/z=579.4 [M+H]⁺. Aliquot was directly transferred to the next step.

Step 2

In the reaction mixture from Step 1, THE (1.5 mL) and LiOH (1M solⁿ in H₂O, 1.5 mL, 20.0 equiv.) were added and stirred for 2 h at 80° C. in a heating block. After cooling to room temperature, the reaction mixture was quenched with 1N HCl and concentrated under vacuum. The crude residue was re-dissolved in 2.0 mL of dimethyl sulfoxide, passed through a 0.45 m syringe filter, and purified by RPTIPLC to afford (R)-6-(7-chloro-3-cyclohexyl-2-methyl-1,1-dioxido-5-phenyl-2,3,4,5-tetrahydrobenzo[f][1,2,5]thiadiazepin-8-yl)-1H-indazole-4-carboxylic acid, Ex.208 (14.4 mg, 34 yield). ESI MS m/z=565.1 [M+H]⁺.

| Example | Structure | MS | NMR |
|---|---|---|---|
| 208 | 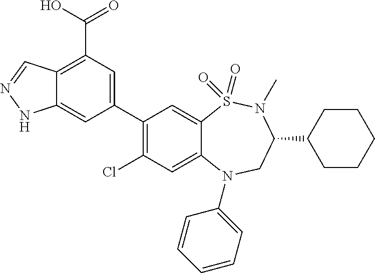 [SMILES: CN1[C@H](C2CCCCC2)CN(c2ccccc2)c2cc(Cl)c(-c3cc(C(=O)O)c4cn[nH]c4c3)cc2S1(=O)=O] | [M + H]⁺ 565.1 | ¹H NMR (500 MHz, DMSO-d6) δ 13.50 (s, 1H), 13.35 (s, 1H), 8.45 (s, 1H), 7.92 (s, 1H), 7.83 (s, 2H), 7.38 − 7.25 (m, 3H), 7.12 − 6.89 (m, 2H), 4.38 (d, J = 16.0 Hz, 1H), 3.88 − 3.58 (m, 1H), 2.71 (s, 3H), 2.01 − 1.84 (m, 2H), 1.78 − 1.52 (m, 4H), 1.30 − 1.08 (m, 3H), 1.06 − 0.90 (m, 2H). |

The following compounds were prepared using a procedure analogous to that described for Ex.208:

| Example | Structure | MS | NMR |
|---|---|---|---|
| 209 | 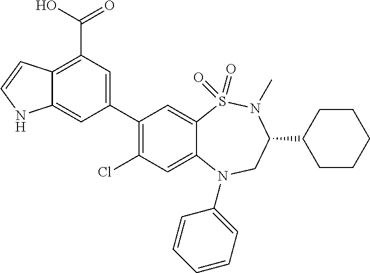 [SMILES: CN1[C@H](C2CCCCC2)CN(c2ccccc2)c2cc(Cl)c(-c3cc(C(=O)O)c4cc[nH]c4c3)cc2S1(=O)=O] | [M + H]⁺ 564.2 | ¹H NMR (400 MHz, DMSO-d6) δ 12.79 (br, 1H), 11.55 (s, 1H), 7.85 − 7.74 (m, 3H), 7.60 (t, J = 2.8 Hz, 1H), 7.31 (t, J = 7.8 Hz, 3H), 7.06 − 6.87 (m, 4H), 4.38 (d, J = 16.0 Hz, 1H), 3.75 − 3.53 (m, 1H), 2.69 (s, 3H), 2.04 − 1.84 (m, 2H), 1.80 − 1.49 (m, 4H), 1.33 − 0.88 (m, 5H). |
| 210 | 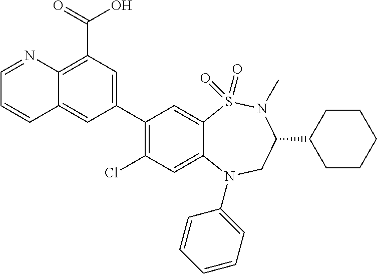 [SMILES: CN1[C@H](C2CCCCC2)CN(c2ccccc2)c2cc(Cl)c(-c3cc(C(=O)O)c4ncccc4c3)cc2S1(=O)=O] | [M + H]⁺ 576.3 | |
| 211 | 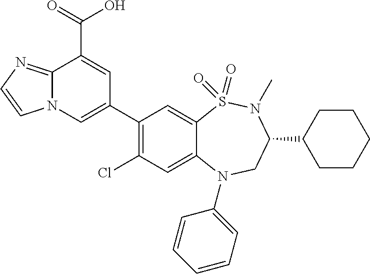 [SMILES: CN1[C@H](C2CCCCC2)CN(c2ccccc2)c2cc(Cl)c(-c3cc(C(=O)O)c4nccn4c3)cc2S1(=O)=O] | [M + H]⁺ 565.4 | |
| 212 | 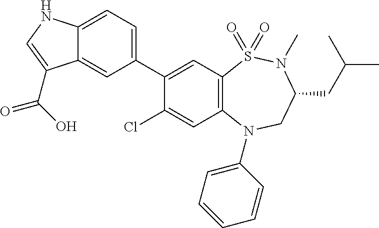 [SMILES: CC(C)C[C@@H]1CN(c2ccccc2)c2cc(Cl)c(-c3ccc4[nH]cc(C(=O)O)c4c3)cc2S(=O)(=O)N1C] | [M + H]⁺ 538.4 | |

### Ex.213: Synthesis of (R)-4-(7-chloro-3-cyclohexyl-2-methyl-1,1-dioxido-5-phenyl-2,3,4,5-tetrahydrobenzo[f][1,2,5]thiadiazepin-8-yl)phthalic acid

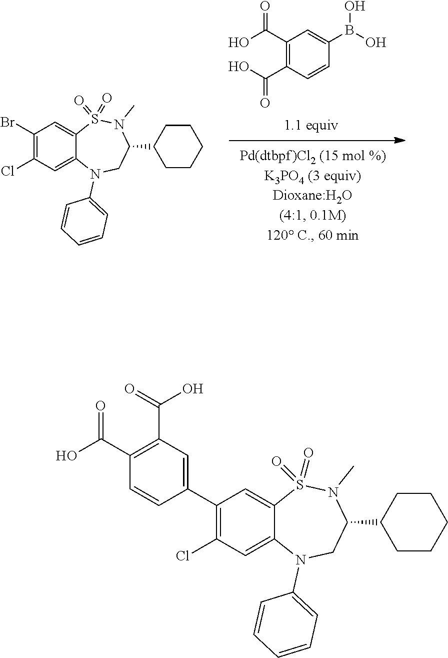 [SMILES: CN1[C@H](C2CCCCC2)CN(c2ccccc2)c2cc(Cl)c(Br)cc2S1(=O)=O ; O=C(O)c1ccc(B(O)O)cc1C(=O)O ; CN1[C@H](C2CCCCC2)CN(c2ccccc2)c2cc(Cl)c(-c3ccc(C(=O)O)c(C(=O)O)c3)cc2S1(=O)=O]

In a 2 mL reaction vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (23.0 mg, 1.0 equiv), 4-boronophthalic acid (10.0 mg, 1.1 equiv, CAS #: 1072946-35-0), [1,1′-bis(di-tert-butylphosphino) ferrocene]dichloropalladium(II) (4.4 mg, 0.15 equiv, CAS #: 95408-45-0) and potassium phosphate tribasic (29.0 mg, 3.0 equiv, CAS #: 7778-53-2) were combined neat under nitrogen atmosphere and followed by addition of 1,4-dioxane (0.290 mL) and water (0.072 mL). The reaction mixture was heated at 120° C. for 1 h under microwave condition. Upon cooling to the room temperature, reaction mixture was quenched with formic acid (0.2 mL) and concentrated and purified by reversed phase C₁₈ flash column chromatography to afford (R)-4-(7-chloro-3-cyclohexyl-2-methyl-1,1-dioxido-5-phenyl-2,3,4,5-tetrahydrobenzo[f][1,2,5]thiadiazepin-8-yl)phthalic acid, Ex.213 (8.6 mg, 33% yield). ESI MS m/z=569.3 [M+H]⁺.

| Example | Structure | MS |
|---|---|---|
| 213 | 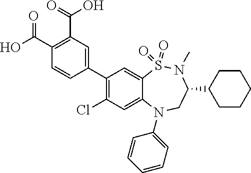 [SMILES: CN1[C@H](C2CCCCC2)CN(c2ccccc2)c2cc(Cl)c(-c3ccc(C(=O)O)c(C(=O)O)c3)cc2S1(=O)=O] | [M + H]⁺ 569.3 |

### Ex.214: Synthesis of(R)-5-(7-chloro-3-isobutyl-2-methyl-1,1-dioxido-5-phenyl-2,3,4,5-tetrahydrobenzo[f][1,2,5]thiadiazepin-8-yl)-2-(2,2,2-trifluoroethoxy)benzoic acid

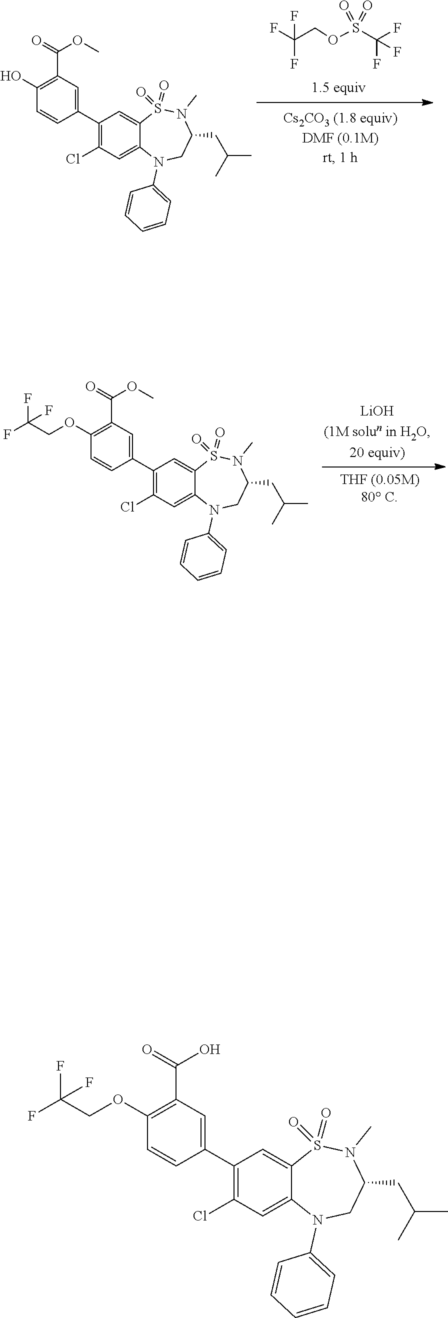 [SMILES: COC(=O)c1cc(-c2cc3c(cc2Cl)N(c2ccccc2)C[C@@H](CC(C)C)N(C)S3(=O)=O)ccc1O ; O=S(=O)(OCC(F)(F)F)C(F)(F)F ; COC(=O)c1cc(-c2cc3c(cc2Cl)N(c2ccccc2)C[C@@H](CC(C)C)N(C)S3(=O)=O)ccc1OCC(F)(F)F ; CC(C)C[C@@H]1CN(c2ccccc2)c2cc(Cl)c(-c3ccc(OCC(F)(F)F)c(C(=O)O)c3)cc2S(=O)(=O)N1C]

Step 1

In a 4 mL vial equipped with a stir bar, methyl (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoate (20.0 mg, 1.0 equiv) and 2,2,2-trifluoroethyl trifluoromethanesulfonate (13.0 mg, 1.5 equiv, CAS #: 6226-25-1) were dissolved in dry DMF (0.4 mL) neat under nitrogen atmosphere. Cesium carbonate (22.0 mg, 1.8 equiv) was added subsequently, and reaction mixture was stirred for 1 h at room temperature. After completion, all volatiles were removed under vacuum to afford methyl (R)-5-(7-chloro-3-isobutyl-2-methyl-1,1-dioxido-5-phenyl-2,3,4,5-tetrahydrobenzo[f][1,2,5]thiadiazepin-8-yl)-2-(2,2,2-trifluoroethoxy)benzoate. ESI MS m/z=611.5 [M+H]⁺. The crude residue was subjected to the next step without further purification.

Step 2

In a 4 mL reaction vial equipped with a stir bar, crude was dissolved in THE (0.8 mL) and followed by addition of LiOH (1M solⁿ in H₂O, 0.75 mL, 20.0 equiv). Reaction mixture was stirred for 2 h at 80 C.

Once completed, reaction mixture was quenched with 1N HCl and concentrated under vacuum. The crude residue was re-dissolved in 2.0 mL of dimethyl sulfoxide, passed through a 0.45 μm syringe filter, and purified by RPTIPLC to afford (R)-5-(7-chloro-3-isobutyl-2-methyl-1,1-dioxido-5-phenyl-2,3,4,5-tetrahydrobenzo[f][1,2,5]thiadiazepin-8-yl)-2-(2,2,2-trifluoroethoxy)benzoic acid, Ex.214 (15.4 mg, 70% yield). ESI MS m/z=597.4 [M+H]⁺.

| Example | Structure | MS | NMR |
|---|---|---|---|
| 214 | 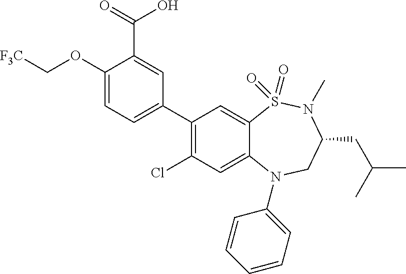 [SMILES: CC(C)C[C@@H]1CN(c2ccccc2)c2cc(Cl)c(-c3ccc(OCC(F)(F)F)c(C(=O)O)c3)cc2S(=O)(=O)N1C] | [M + H]+ 597.4 | ¹H NMR (400 MHz, DMSO-d6) δ 13.01 (s, 1H), 7.80 (d, J = 2.4 Hz 1H), 7.78 (s, 1H), 7.71 (dd, J = 8.6, 2.5 Hz, 1H), 7.45 (s, 1H), 7.35 (d, J = 8.7 Hz, 1H), 7.31 − 7.22 (m, 2H), 6.92 − 6.82 (m, 3H), 4.89 (q, J = 8.8 Hz, 2H), 4.09 (d, J = 16.1 Hz, 1H), 3.97 − 3.81 (m, 1H), 3.53 − 3.36 (m, 1H), 2.59 (s, 3H), 1.76 − 1.63 (m, 1H), 1.61 − 1.48 (m, 1H), 1.41 − 1.29 (m, 1H), 0.93 (d, J = 6.6 Hz, 3H), 0.89 (d, J = 6.5 Hz, 3H) |

### Ex.215: Synthesis of (R)-4-(7-chloro-3-isobutyl-2-methyl-1,1-dioxido-5-phenyl-2,3,4,5-tetrahydrobenzo[f][1,2,5]thiadiazepin-8-yl)-[1,1′-biphenyl]-2-carboxylic acid

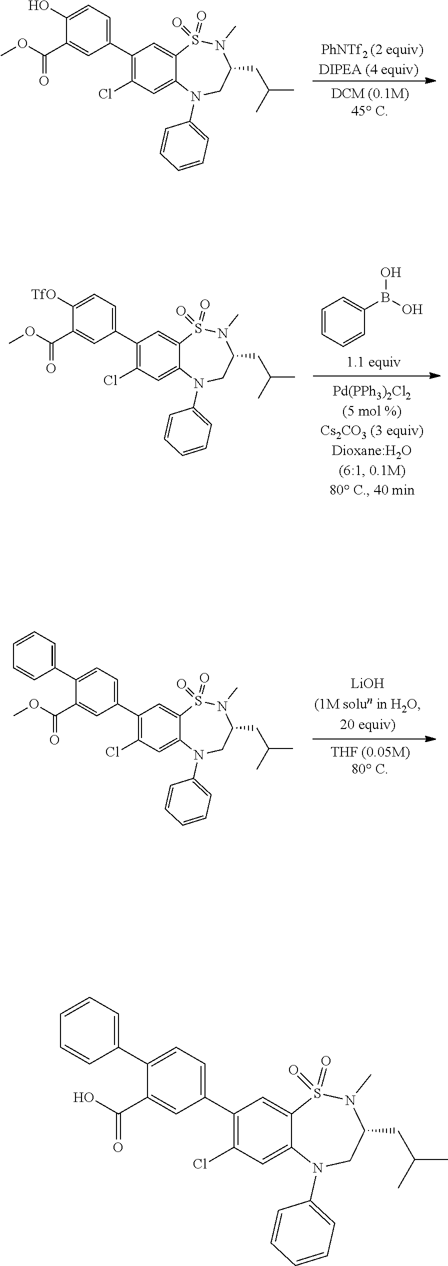

Step 1

In a 8 mL reaction vial equipped with a stir bar, methyl (R)-5-(7-chloro-3-isobutyl-2-methyl-1,1-dioxido-5-phenyl-2,3,4,5-tetrahydrobenzo[f][1,2,5]thiadiazepin-8-yl)-2-hydroxybenzoate (120.0 mg, 1.0 equiv.) was dissolved in DCM (2.268 mL) at room temperature, followed by addition of triethylamine (0.126 mL, 4.0 equiv.). To this stirred solution, 1,1,1-trifluoro-N-phenyl-N-((trifluoromethyl)sulfonyl)methanesulfonamide (162 mg, 2.0 equiv., CAS #82113-65-3) was added and stirred for 5 h at 45° C. 40% conversion was observed. Added additional 4.0 equiv. of (0.126 mL) triethylamine and 2 equiv. of 1,1,1-trifluoro-N-phenyl-N-((trifluoromethyl)sulfonyl) methanesulfonamide (162 mg) and stirred for overnight. Reaction mixture was concentrated under vacuum and crude was subjected to silica gel flash chromatography to afford methyl (R)-5-(7-chloro-3-isobutyl-2-methyl-1,1-dioxido-5-phenyl-2,3,4,5-tetrahydrobenzo[f][1,2,5]thiadiazepin-8-yl)-2-(((trifluoromethyl)sulfonyl)oxy)benzoate in quantitative yield. ESI MS m/z=661.2 [M+H]⁺.

Step 2 & 3

In 4 mL reaction vial equipped with a stir bar methyl (R)-5-(7-chloro-3-isobutyl-2-methyl-1,1-dioxido-5-phenyl-2,3,4,5-tetrahydrobenzo[f][1,2,5]thiadiazepin-8-yl)-2-(((trifluoromethyl)sulfonyl)oxy)benzoate (30.0 mg, 1.0 equiv.), phenyl boronic acid (6.1 mg, 1.1 equiv., CAS #: 98-80-6), bis(triphenylphosphine)palladium(II) chloride (1.5 mg, 0.05 equiv.) and cesium carbonate (44.4 mg, 3.0 equiv.) were combined neat under nitrogen atmosphere, followed by addition of dioxane (0.29 mL) and water (0.07 mL). Reaction mixture was heated at 80° C. for 4 h in a heating block. 50% conversion was observed. Added additional amount of 1.1 equiv. of phenyl boronic acid (6.1 mg) and a pinch of Pd catalyst and continued stirring overnight at 80° C.

Once completed, THE (1.0 mL) and LiOH (1M solⁿ in H₂O, 0.9 mL, 20.0 equiv.) were added to the reaction mixture and stirred overnight at 80° C. After cooling to room temperature, the reaction mixture was quenched with 1N HCl and concentrated under vacuum. The crude residue was re-dissolved in 2.0 mL of dimethyl sulfoxide, passed through a 0.45 m syringe filter, and purified by RPTIPLC to afford (R)-4-(7-chloro-3-isobutyl-2-methyl-1,1-dioxido-5-phenyl-2,3,4,5-tetrahydrobenzo[f][1,2,5]thiadiazepin-8-yl)-[1,1′-biphenyl]-2-carboxylic acid, Ex.215 (6.5 mg, 2500 yield). ESI MS m/z=575.4 [M+H]⁺.

| Example | Structure | MS | NMR |
|---|---|---|---|
| 215 | 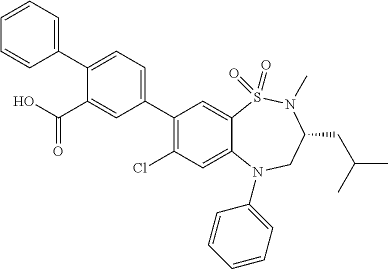 [SMILES: CC(C)C[C@@H]1CN(c2ccccc2)c2cc(Cl)c(-c3ccc(-c4ccccc4)c(C(=O)O)c3)cc2S(=O)(=O)N1C] | [M + H]⁺ | ¹H NMR (400 MHz, DMSO-d6) δ 7.85 (s, 1H), 7.83 − 7.80 (m, 1H), 7.73 (dd, J = 7.9, 2.1 Hz, 1H), 7.52 (d, J = 7.9 Hz, 1H), 7.49 − 7.35 (m, 6H), 7.33 − 7.24 (m, 2H), 6.94 − 6.85 (m, 3H), 4.10 (d, J = 16.1 Hz, 1H), 3.97 − 3.81 (m, 1H), 2.61 (s, 3H), 1.77 − 1.64 (m, 1H), 1.63 − 1.49 (m, 1H), 1.42 − 1.31 (m, 1H), 0.93 (d, J = 6.6 Hz, 3H), 0.89 (d, J = 6.5 Hz, 3H). |

The following compounds were prepared using a procedure analogous to that described for Ex.215. Ex.218 was isolated as a byproduct during the preparation of Ex.217.

| Example | Structure | MS |
|---|---|---|
| 216 | 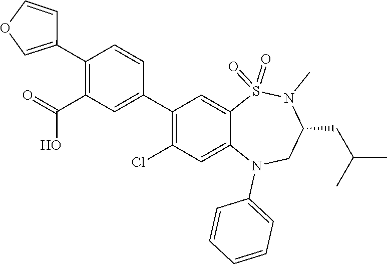 [SMILES: CC(C)C[C@@H]1CN(c2ccccc2)c2cc(Cl)c(-c3ccc(-c4ccoc4)c(C(=O)O)c3)cc2S(=O)(=O)N1C] | [M + H]⁺ 565.3 |
| 217 | 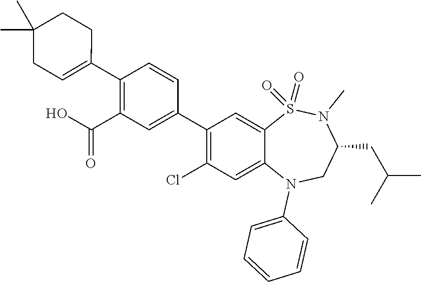 [SMILES: CC(C)C[C@@H]1CN(c2ccccc2)c2cc(Cl)c(-c3ccc(C4=CCC(C)(C)CC4)c(C(=O)O)c3)cc2S(=O)(=O)N1C] | [M + H]⁺ 607.6 |
| 218 | 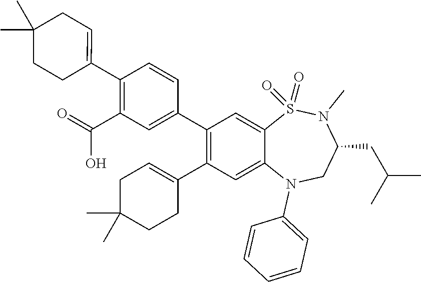 [SMILES: CC(C)C[C@@H]1CN(c2ccccc2)c2cc(C3=CCC(C)(C)CC3)c(-c3ccc(C4=CCC(C)(C)CC4)c(C(=O)O)c3)cc2S(=O)(=O)N1C] | [M + H]⁺ 681.7 |

### Ex. 219: Synthesis of (R)-5-(7-chloro-3-isobutyl-2-methyl-1,1-dioxido-5-phenyl-2,3,4,5-tetrahydrobenzo[f][1,2,5]thiadiazepin-8-yl)-2-(piperidin-1-yl)benzoic acid

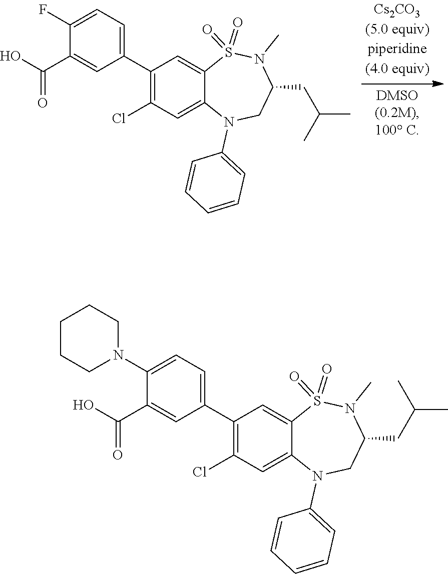 [SMILES: CC(C)C[C@@H]1CN(c2ccccc2)c2cc(Cl)c(-c3ccc(F)c(C(=O)O)c3)cc2S(=O)(=O)N1C ; CC(C)C[C@@H]1CN(c2ccccc2)c2cc(Cl)c(-c3ccc(N4CCCCC4)c(C(=O)O)c3)cc2S(=O)(=O)N1C]

To a 2-dram glass vial containing a magnetic stir bar and (R)-5-(7-chloro-3-isobutyl-2-methyl-1,1-dioxido-5-phenyl-2,3,4,5-tetrahydrobenzo[f][1,2,5]thiadiazepin-8-yl)-2-fluorobenzoic acid (68 mg, 0.131 mmol) were added DMSO (0.65 mL), piperidine (0.02 mL, 0.197 mmol) and cesium carbonate (128 mg, 0.393 mmol). The resulting mixture was heated to 100° C. and stirred overnight, then purified by RP-HPLC to afford (R)-5-(7-chloro-3-isobutyl-2-methyl-1,1-dioxido-5-phenyl-2,3,4,5-tetrahydrobenzo[f][1,2,5]thiadiazepin-8-yl)-2-(piperidin-1-yl)benzoic acid, Ex.219 (5 mg, 7%) as a white solid:

| Example | Structure | MS |
|---|---|---|
| 219 | 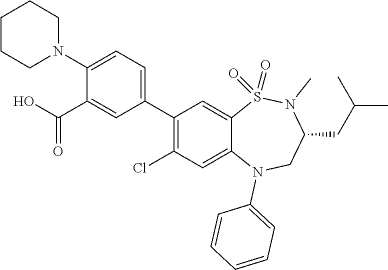 [SMILES: CC(C)C[C@@H]1CN(c2ccccc2)c2cc(Cl)c(-c3ccc(N4CCCCC4)c(C(=O)O)c3)cc2S(=O)(=O)N1C] | [M + H]⁺ |

### Ex.220: Synthesis of(R)-N-((3-(7-chloro-3-isobutyl-2-methyl-1,1-dioxido-5-phenyl-2,3,4,5-tetrahydrobenzo[f][1,2,5]thiadiazepin-8-yl)phenyl)sulfonyl)acetamide

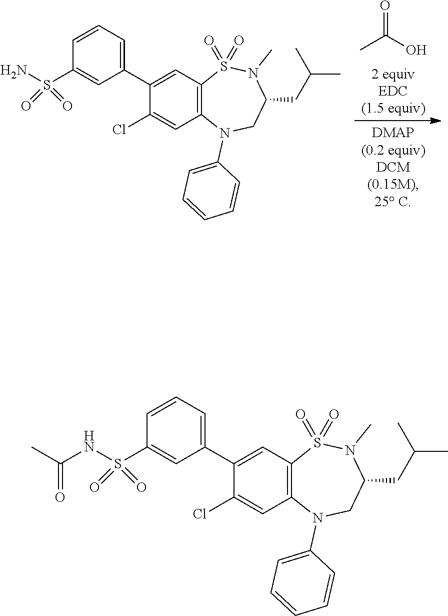

In a 2 mL vial equipped with a stir bar, (R)-3-(7-chloro-3-isobutyl-2-methyl-1,1-dioxido-5-phenyl-2,3,4,5-tetrahydrobenzo[f][1,2,5]thiadiazepin-8-yl)benzenesulfonamide (14.8 mg, 1.0 equiv.) was dissolved in dry DCM (0.185 ml) under nitrogen atmosphere. Subsequently, acetic acid (3.17 μl, 2.0 equiv.), 3-(((ethylimino)methylene)amino)-N,N-dimethylpropan-1-amine hydrochloride (7.97 mg, 1.5 equiv.), and N,N-dimethylpyridin-4-amine (0.677 mg, 0.2 equiv.) were added and reaction mixture was stirred at room temperature. After 12 h, the reaction was quenched with a few drops of 1N HCl and concentrated under vacuum. The crude residue was re-dissolved in 2.0 mL of dimethyl sulfoxide, passed through a 0.45 μm syringe filter, and purified by RPHPLC to afford (R)-N-((3-(7-chloro-3-isobutyl-2-methyl-1,1-dioxido-5-phenyl-2,3,4,5-tetrahydrobenzo[f][1,2,5]thiadiazepin-8-yl)phenyl)sulfonyl)acetamide, Ex.220 (8.5 mg, 53% yield). ESI MS m/z=576.3 [M+H]⁺.

| Example | Structure | MS | NMR |
|---|---|---|---|
| 220 | 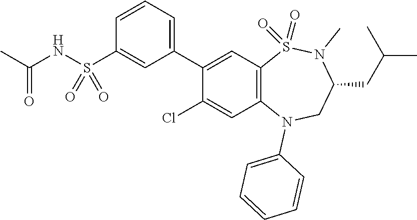 [SMILES: CC(=O)NS(=O)(=O)c1cccc(-c2cc3c(cc2Cl)N(c2ccccc2)C[C@@H](CC(C)C)N(C)S3(=O)=O)c1] | [M + H]⁺ 576.3 | ¹H NMR (400 MHz, DMSO-d6) δ 12.19 (s, 1H), 8.04-7.92 (m, 2H), 7.92-7.84 (m, 1H), 7.81 (s, 1H), 7.76 (t, J = 7.8 Hz, 1H), 7.44 (s, 1H), 7.28 (dd, J = 8.7, 7.2 Hz, 2H), 6.96-6.88 (m, 3H), 4.11 (d, J = 16.1 Hz, 1H), 3.93-3.81 (m, 1H), 3.58-3.41 (m, 1H), 2.62 (s, 3H), 1.93 (s, 3H), 1.75-1.64 (m, 1H), 1.63-1.50 (m, 1H), 1.41-1.31 (m, 1H), 0.92 (d, J = 6.6 Hz, 3H), 0.89 (d, J = 6.5 Hz, 3H). |

The following compounds were prepared using a procedure analogous to that described for Ex.220.

| Example | Structure | MS |
|---|---|---|
| 221 | 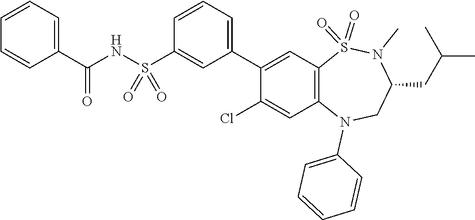 [SMILES: CC(C)C[C@@H]1CN(c2ccccc2)c2cc(Cl)c(-c3cccc(S(=O)(=O)NC(=O)c4ccccc4)c3)cc2S(=O)(=O)N1C] | [M + H]⁺ 638.4 |
| 222 | 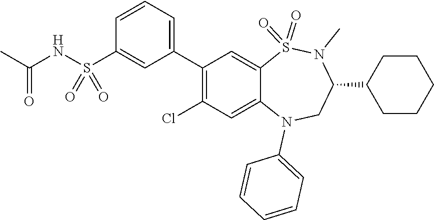 [SMILES: CC(=O)NS(=O)(=O)c1cccc(-c2cc3c(cc2Cl)N(c2ccccc2)C[C@@H](C2CCCCC2)N(C)S3(=O)=O)c1] | [M + H]⁺ 602.2 |
| 223 | 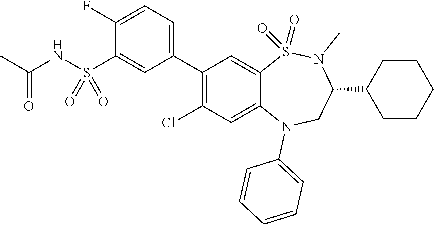 [SMILES: CC(=O)NS(=O)(=O)c1cc(-c2cc3c(cc2Cl)N(c2ccccc2)C[C@@H](C2CCCCC2)N(C)S3(=O)=O)ccc1F] | [M + H]⁺ 620.2 |
| 224 | 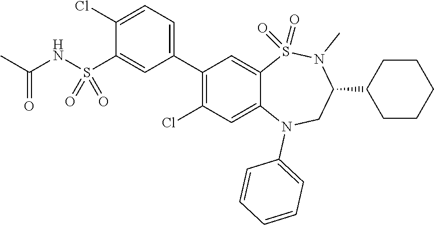 [SMILES: CC(=O)NS(=O)(=O)c1cc(-c2cc3c(cc2Cl)N(c2ccccc2)C[C@@H](C2CCCCC2)N(C)S3(=O)=O)ccc1Cl] | [M + H]⁺ 636.2 |
| 225 | 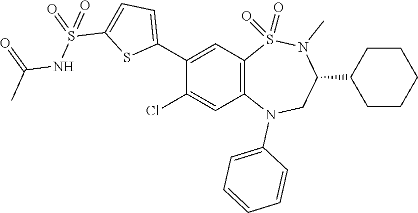 [SMILES: CC(=O)NS(=O)(=O)c1ccc(-c2cc3c(cc2Cl)N(c2ccccc2)C[C@@H](C2CCCCC2)N(C)S3(=O)=O)s1] | [M + H]⁺ 608.1 |
| 226 | 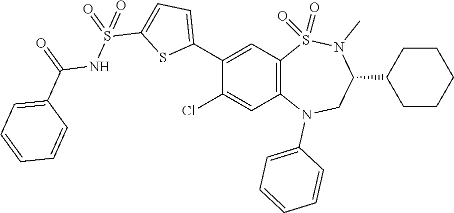 [SMILES: CN1[C@H](C2CCCCC2)CN(c2ccccc2)c2cc(Cl)c(-c3ccc(S(=O)(=O)NC(=O)c4ccccc4)s3)cc2S1(=O)=O] | [M + H]⁺ 670.0 |
| 227 | 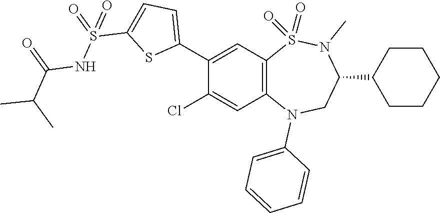 [SMILES: CC(C)C(=O)NS(=O)(=O)c1ccc(-c2cc3c(cc2Cl)N(c2ccccc2)C[C@@H](C2CCCCC2)N(C)S3(=O)=O)s1] | [M + H]⁺ 636.2 |
| 228 | 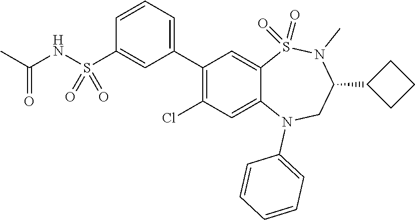 [SMILES: CC(=O)NS(=O)(=O)c1cccc(-c2cc3c(cc2Cl)N(c2ccccc2)C[C@@H](C2CCC2)N(C)S3(=O)=O)c1] | [M + H]⁺ 574.1 |

The following compound, Ex.229, was prepared starting from (R)-2-amino-5-(7-chloro-3-cyclohexyl-2-methyl-1,1-dioxido-dioxido-5-phenyl-2,3,4,5-tetrahydrobenzo[f][1,2,5]thiadiazepin-8-yl)benzoic acid, Ex.157, using a procedure similar to that used for Ex.220:

| Example | Structure | MS |
|---|---|---|
| 229 | 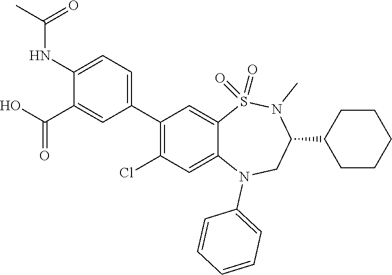 [SMILES: CC(=O)Nc1ccc(-c2cc3c(cc2Cl)N(c2ccccc2)C[C@@H](C2CCCCC2)N(C)S3(=O)=O)cc1C(=O)O] | [M + H]⁺ 582.3 |

### Ex.230: Synthesis of(R)-3-(7-chloro-3-isobutyl-2-methyl-1,1-dioxido-5-phenyl-2,3,4,5-tetrahydrobenzo[f][1,2,5]thiadiazepin-8-yl)benzenesulfonic acid

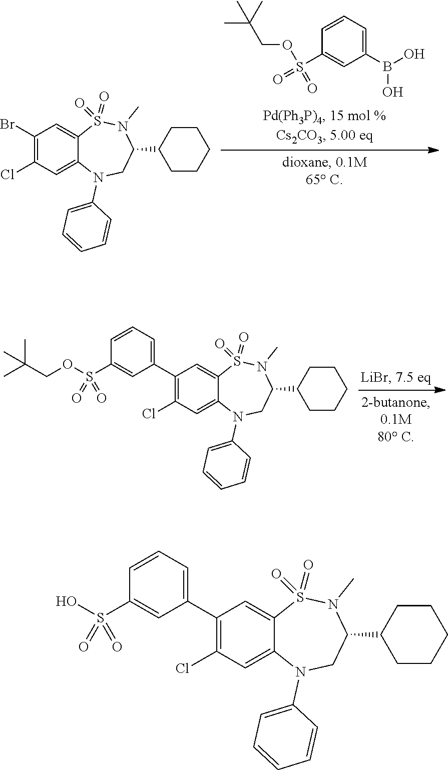 [SMILES: CC(C)(C)COS(=O)(=O)c1cccc(B(O)O)c1 ; CN1[C@H](C2CCCCC2)CN(c2ccccc2)c2cc(Cl)c(Br)cc2S1(=O)=O ; CN1[C@H](C2CCCCC2)CN(c2ccccc2)c2cc(Cl)c(-c3cccc(S(=O)(=O)OCC(C)(C)C)c3)cc2S1(=O)=O ; CN1[C@H](C2CCCCC2)CN(c2ccccc2)c2cc(Cl)c(-c3cccc(S(=O)(=O)O)c3)cc2S1(=O)=O]

Step 1

To a 2-dram glass vial containing (R)-8-bromo-7-chloro-3-cyclohexyl-2-methyl-5-phenyl-2,3,4,5-tetrahydrobenzo[f][1,2,5]thiadiazepine 1,1-dioxide (26 mg, 0.054 mmol) and a magnetic stir bar were added (3-((neopentyloxy)sulfonyl)phenyl)boronic acid (16 mg, 0.059 mmol), cesium carbonate (88 mg, 0.269 mmol) and dioxane (0.5 mL). The resulting mixture was sparged with nitrogen, then Pd(Ph₃P)₄ (9.31 mg, 8.06 μmol) was added, the vessel capped, sealed with parafilm, and heated to 65° C. After stirring overnight, the reaction mixture was diluted with DCM, filtered and concentrated, then purified by RP-HPLC to afford neopentyl (R)-3-(7-chloro-3-cyclohexyl-2-methyl-1,1-dioxido-5-phenyl-2,3,4,5-tetrahydrobenzo[f][1,2,5]thiadiazepin-8-yl)benzenesulfonate (12 mg, 35%) as a colorless solid. ESI-MS m/z=631.3 [M+H]⁺.

Step 2

To a 2-dram glass vial containing neopentyl (R)-3-(7-chloro-3-cyclohexyl-2-methyl-1,1-dioxido-5-phenyl-2,3,4,5-tetrahydrobenzo[f][1,2,5]thiadiazepin-8-yl)benzenesulfonate (12 mg, 0.019 mmol) were added lithium bromide (62 mg, 0.714 mmol) and a magnetic stir bar, followed by 2-butanone (0.190 ml). After sonication, a yellow solution was obtained. The reaction vessel was heated to 80° C.; after stirring 24 h, additional LiBr (55 mg, 0.613 mmol) was added, and the temperature was increased to 95° C. After stirring an additional 12 h, the reaction mixture was concentrated, quenched with 1 N HCl (0.3 mL), diluted with water, and extracted with EtOAc (3×0.75 mL). The combined organics were concentrated and purified by RP-HPLC to afford (R)-3-(7-chloro-3-cyclohexyl-2-methyl-1,1-dioxido-5-phenyl-2,3,4,5-tetrahydrobenzo[f][1,2,5]thiadiazepin-8-yl)benzenesulfonic acid, Ex.230 (1.2 mg, 11%) as a colorless solid:

| Example | Structure | MS |
|---|---|---|
| 230 | 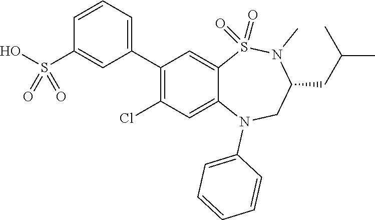 [SMILES: CC(C)C[C@@H]1CN(c2ccccc2)c2cc(Cl)c(-c3cccc(S(=O)(=O)O)c3)cc2S(=O)(=O)N1C] | [M + H]⁺ 535.2 |

Likewise, the following compound was prepared by a procedure identical to that described above, except that in Step 1, neopentyl 2-chloro-5-(4,4,5,5-tetramethyl-1,3,2-dioxaborolan-2-yl)benzenesulfonate was used in place of (3-((neopentyloxy) sulfonyl)phenyl)boronic acid:

| Example | Structure | MS |
|---|---|---|
| 231 | 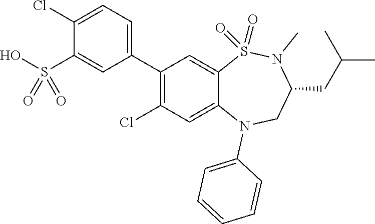 [SMILES: CC(C)C[C@@H]1CN(c2ccccc2)c2cc(Cl)c(-c3ccc(Cl)c(S(=O)(=O)O)c3)cc2S(=O)(=O)N1C] | [M − H]⁻ 593.2 |

### Ex.232: Synthesis of (R)-(3-(7-chloro-3-cyclohexyl-2-methyl-1,1-dioxido-5-phenyl-2,3,4,5-tetrahydrobenzo[f][1,2,5]thiadiazepin-8-yl)phenyl)phosphonic acid

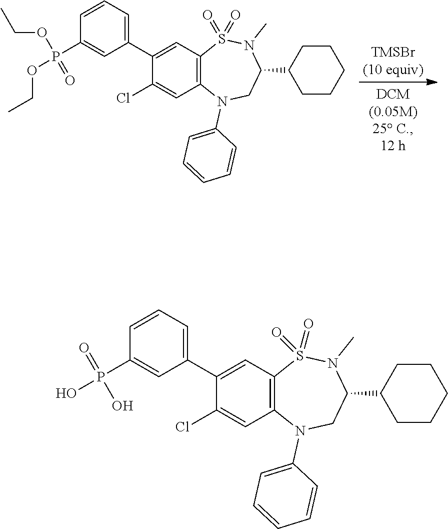 [SMILES: CCOP(=O)(OCC)c1cccc(-c2cc3c(cc2Cl)N(c2ccccc2)C[C@@H](C2CCCCC2)N(C)S3(=O)=O)c1 ; CN1[C@H](C2CCCCC2)CN(c2ccccc2)c2cc(Cl)c(-c3cccc(P(=O)(O)O)c3)cc2S1(=O)=O]

In a 8 mL vial equipped with a stir bar, diethyl (R)-(3-(7-chloro-3-cyclohexyl-2-methyl-1,1-dioxido-5-phenyl-2,3,4,5-tetrahydrobenzo[f][1,2,5]thiadiazepin-8-yl)phenyl)phosphonate (48 mg, 1.0 equiv.) was dissolved in dry DCM (1.6 mL), followed by dropwise addition of bromotrimethylsilane (0.1 mL, 10 equiv. CAS #2857-97-8). Reaction mixture was stirred at room temperature (25° C.) and reaction progress was monitored through LC-MS. After 12 h, reaction mixture was concentrated under vacuum and crude was purified through reversed phase flash column chromatography to afford (R)-(3-(7-chloro-3-cyclohexyl-2-methyl-1,1-dioxido-5-phenyl-2,3,4,5-tetrahydrobenzo[f][1,2,5]thiadiazepin-8-yl)phenyl)phosphonic acid, Ex.232 (23 mg, 53% yield). ESI MS m/z=561.4 [M+H]⁺.

| Example | Structure | MS | NMR |
|---|---|---|---|
| 232 | 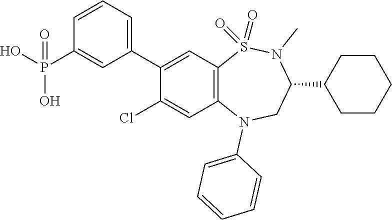 [SMILES: CN1[C@H](C2CCCCC2)CN(c2ccccc2)c2cc(Cl)c(-c3cccc(P(=O)(O)O)c3)cc2S1(=O)=O] | [M + H]+ 561.4 | ¹H NMR (400 MHz, DMSO-d6) δ 11.18 (br, 1H), 7.80-7.68 (m, 3H), 7.68-7.61 (m, 1H), 7.57 (td, J = 7.8, 3.6 Hz, 1H), 7.38-7.18 (m, 3H), 7.10-6.88 (m, 3H), 4.37 (d, J = 16.0 Hz, 1H), 3.85-3.58 (m, 2H), 2.70 (s, 3H), 2.02-1.58 (m, 6H), 1.32-0.84 (m, 5H). |

The following compounds were prepared using a procedure analogous to that described for Ex.232:

| Example | Structure | MS |
|---|---|---|
| 233 | 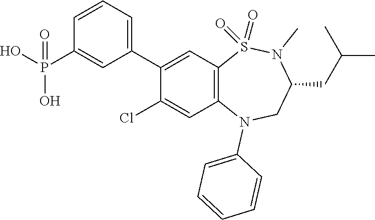 [SMILES: CC(C)C[C@@H]1CN(c2ccccc2)c2cc(Cl)c(-c3cccc(P(=O)(O)O)c3)cc2S(=O)(=O)N1C] | [M + H]+ 535.4 |
| 234 | 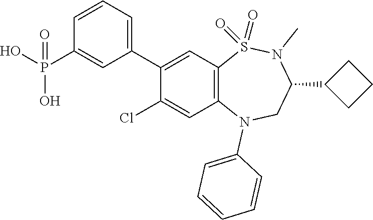 [SMILES: CN1[C@H](C2CCC2)CN(c2ccccc2)c2cc(Cl)c(-c3cccc(P(=O)(O)O)c3)cc2S1(=O)=O] | [M + H]+ 533.2 |

### Ex.235: Synthesis of(R)-2-(((benzyloxy)carbonyl)amino)-5-(3-cyclohexyl-7-fluoro-2-methyl-1,1-dioxido-5-phenyl-2,3,4,5-tetrahydrobenzo[f][1,2,5]thiadiazepin-8-yl)benzoic acid

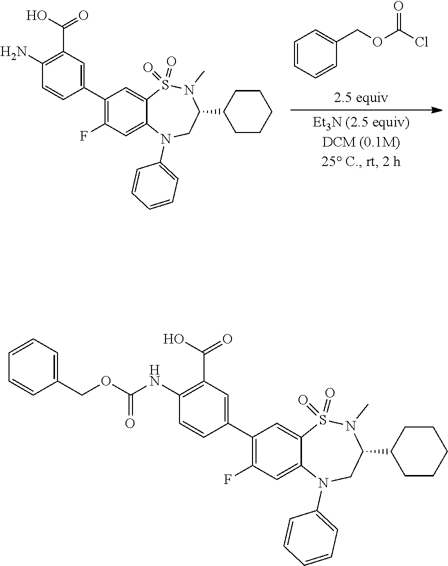 [SMILES: CN1[C@H](C2CCCCC2)CN(c2ccccc2)c2cc(F)c(-c3ccc(N)c(C(=O)O)c3)cc2S1(=O)=O ; O=C(Cl)OCc1ccccc1 ; CN1[C@H](C2CCCCC2)CN(c2ccccc2)c2cc(F)c(-c3ccc(NC(=O)OCc4ccccc4)c(C(=O)O)c3)cc2S1(=O)=O]

In a 8 mL vial equipped with a stir bar, (R)-2-amino-5-(3-cyclohexyl-7-fluoro-2-methyl-1,1-dioxido-5-phenyl-2,3,4,5-tetrahydrobenzo[f][1,2,5]thiadiazepin-8-yl)benzoic acid (120.0 mg, 1.0 equiv.) was dissolved in dry DCM (2.9 mL) and followed by addition of benzyl chloroformate (82 μl, 2.5 equiv., CAS #501-53-1) and triethylamine (80 μl, 2.5 equiv.) at 0° C. Reaction mixture was stirred for 5 minutes at 0° C. and then ice bath was removed and continue stirring at 25° C. for 2 h. Then, mixture was concentrated under vacuum and the crude residue was re-dissolved in 4.0 mL of dimethyl sulfoxide and loaded in a 100 g C18 gold column for reversed phase flash chromatography purification to afford (R)-2-(((benzyloxy)carbonyl)amino)-5-(3-cyclohexyl-7-fluoro-2-methyl-1,1-dioxido-5-phenyl-2,3,4,5-tetrahydrobenzo[f][1,2,5]thiadiazepin-8-yl)benzoic acid, Ex.235 (105 mg, 70% yield). ESI MS m/z=658.1 [M+H]⁺.

| Example | Structure | MS |
|---|---|---|
| 235 | 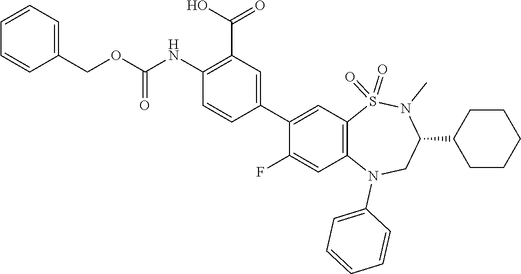 [SMILES: CN1[C@H](C2CCCCC2)CN(c2ccccc2)c2cc(F)c(-c3ccc(NC(=O)OCc4ccccc4)c(C(=O)O)c3)cc2S1(=O)=O] | [M + H]⁺ 658.1 |

The following compounds were prepared using a procedure analogous to that described for Ex.235:

| Example | Structure | MS | NMR |
|---|---|---|---|
| 236 | 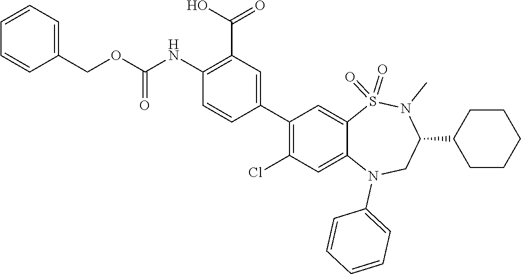 [SMILES: CN1[C@H](C2CCCCC2)CN(c2ccccc2)c2cc(Cl)c(-c3ccc(NC(=O)OCc4ccccc4)c(C(=O)O)c3)cc2S1(=O)=O] | [M + H]⁺ 674.2 | ¹H NMR (500 MHz, MeOD- d4) δ 8.49 (d, J = 8.7 Hz, 1H), 8.14 (d, J = 2.3 Hz, 1H), 7.79 (s, 1H), 7.65 (dd, J = 8.7, 2.3 Hz, 1H), 7.47-7.42 (m, 2H), 7.42-7.30 (m, 5H), 7.14-7.02 (m, 4H), 5.23 (s, 2H), 4.38 (d, J = 15.6 Hz, 1H), 3.93 (br, 1H), 2.83 (s, 3H), 2.13-1.62 (m, 6H), 1.68-0.23 (m, 5H). |

### Ex.237: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-2-(3-phenylureido)benzoic acid

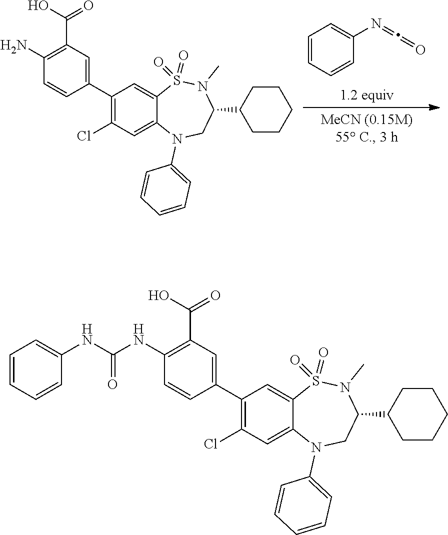

In a 2 mL vial equipped with a stir bar, (R)-2-amino-5-(7-chloro-3-cyclohexyl-2-methyl-1,1-dioxido-5-phenyl-2,3,4,5-tetrahydrobenzo[f][1,2,5]thiadiazepin-8-yl)benzoic acid (15 mg, 1.0 equiv.) was dissolved in dry MeCN (0.2 mL) under nitrogen, followed by addition of phenyl isocyanate (8.75 mg, 2.5 equiv., CAS #103-71-9) at room temperature. Then, the reaction vial was heated at 55° C. in a heating block for 3 h. Reaction progress was monitored through LC-MS. After completion, reaction mixture was concentrated under vacuum and the crude residue was re-dissolved in 2.0 mL of dimethyl sulfoxide, passed through a 0.45 m syringe filter, and purified by RPHIPLC to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-(3-phenylureido)benzoic acid, Ex.237 (7.2 mg, 400 yield). ESI MS m/z=659.4 [M+H]H.

| Example | Structure | MS | NMR |
|---|---|---|---|
| 237 | 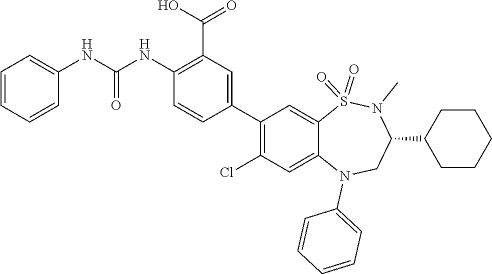 [SMILES: CN1[C@H](C2CCCCC2)CN(c2ccccc2)c2cc(Cl)c(-c3ccc(NC(=O)Nc4ccccc4)c(C(=O)O)c3)cc2S1(=O)=O] | [M + H]⁺ 659.4 | ¹H NMR (400 MHz, DMSO-d6) δ 13.65 (br, 1H), 10.63 (br, 1H), 9.86 (s, 1H), 8.49 (d, J = 8.8 Hz, 1H), 8.05 (d, J = 2.4 Hz, 1H), 7.75 (s, 1H), 7.68 (dd, J = 8.8, 2.4 Hz, 1H), 7.58- 7.47 (m, 2H), 7.38-7.19 (m, 5H), 7.09-6.86 (m, 4H), 4.37 (d, J = 16.0 Hz, 1H), 3.81-3.51 (m, 1H), 2.69 (s, 3H), 2.03-1.83 (m, 2H), 1.85-1.50 (m, 4H), 1.36-0.88 (m, 5H). |

### Ex.238: Synthesis of (R)-3-((tert-butoxycarbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylic acid

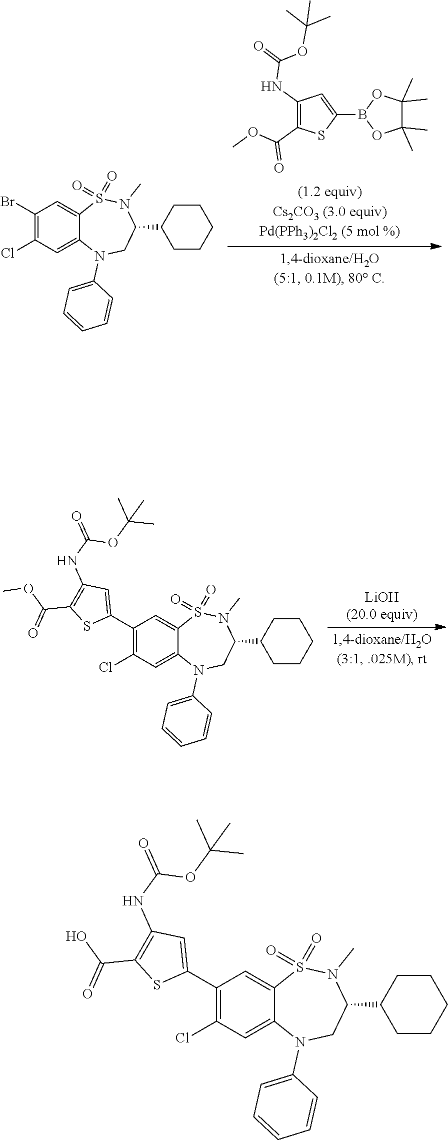

Step 1

In a 40 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (908 mg, 1.877 mmol, 1.0 equiv) was combined neat with methyl 3-((tert-butoxycarbonyl)amino)-5-(4,4,5,5-tetramethyl-1,3,2-dioxaborolan-2-yl)thiophene-2-carboxylate (863 mg, 2.252 mmol, 1.2 equiv, CAS #2377606-49-8), cesium carbonate (1.834 g, 5.63 mmol, 3.0 equiv), and bis(triphenylphosphine)palladium(II) chloride (65.9 mg, 0.094 mmol, 5 mol %) under a nitrogen atmosphere. Next, 1,4-dioxane (10.4 mL) and water (2.09 mL) were added, and the vial was sealed with electrical tape. The reaction mixture was heated at 80° C. for 2 h. Upon cooling to room temperature, the reaction mixture was concentrated and purified directly by silica gel column chromatography to afford methyl (R)-3-((tert-butoxycarbonyl)amino)-5-(7-chloro-3-cyclohexyl-2-methyl-1,1-dioxido-dioxido-5-phenyl-2,3,4,5-tetrahydrobenzo[f][1,2,5]thiadiazepin-8-yl)thiophene-2-carboxylate (525 mg, 42% yield). ESI MS m/z=606.1 [M-C₄H₈+H]⁺, 560.0 [M-C₄Hs-CO₂+H]⁺.

Step 2

In a 20 mL vial equipped with a stir bar, methyl (R)-3-((tert-butoxycarbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate (15 mg, 0.023 mmol, 1.0 equiv) was dissolved in 1,4-dioxane (0.68 mL) and water (0.23 mL). Next, lithium hydroxide (10.9 mg, 0.454 mmol, 20 equiv) was added and the mixture was stirred at room temperature until LCMS analysis indicated full consumption of the starting material. The reaction mixture was quenched with 250 μL of formic acid and purified by RPHPLC to afford (R)-3-((tert-butoxycarbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylic acid, Ex.238 (6.44 mg, 44%). ESI MS m/z=590.1 [M-C₄H₈+H]. ¹H NMR (400 MHz, CD₃CN) δ 9.34 (s, 1H), 8.12 (s, 1H), 8.00 (s, 1H), 7.42-7.33 (m, 2H), 7.16-7.05 (m, 4H), 4.30 (d, J=15.9 Hz, 1H), 3.89 (s, 1H), 3.34 (t, J=10.8 Hz, 1H), 2.79 (s, 3H), 2.03 (d, J=13.2 Hz, 1H), 1.87 (d, J=12.4 Hz, 1H), 1.79-1.65 (m, 4H), 1.54 (s, 9H), 1.33-1.14 (m, 3H), 1.07-0.97 (m, 2H).

| Example | Structure | MS | NMR |
|---|---|---|---|
| 238 | 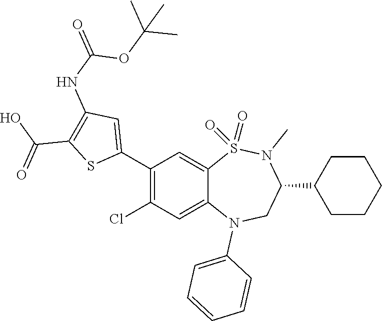 [SMILES: CN1[C@H](C2CCCCC2)CN(c2ccccc2)c2cc(Cl)c(-c3cc(NC(=O)OC(C)(C)C)c(C(=O)O)s3)cc2S1(=O)=O] | [M − C₄H₈ + H]⁺ 590.1 | ¹H NMR (400 MHz, CD₃CN) δ 9.34 (s, 1H), 8.12 (s, 1H), 8.00 (s, 1H), 7.42-7.33 (m, 2H), 7.16-7.05 (m, 4H), 4.30 (d, J = 15.9 Hz, 1H), 3.89 (s, 1H), 3.34 (t, J = 10.8 Hz, 1H), 2.79 (s, 3H), 2.03 (d, J = 13.2 Hz, 1H), 1.87 (d, J = 12.4 Hz, 1H), 1.79-1.65 (m, 4H), 1.54 (s, 9H), 1.33-1.14 (m, 3H), 1.07-0.97 (m, 2H) |

### Ex.239: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-3-(((2-methoxy-2-methylpropoxy)carbonyl)amino)thiophene-2-carboxylic acid

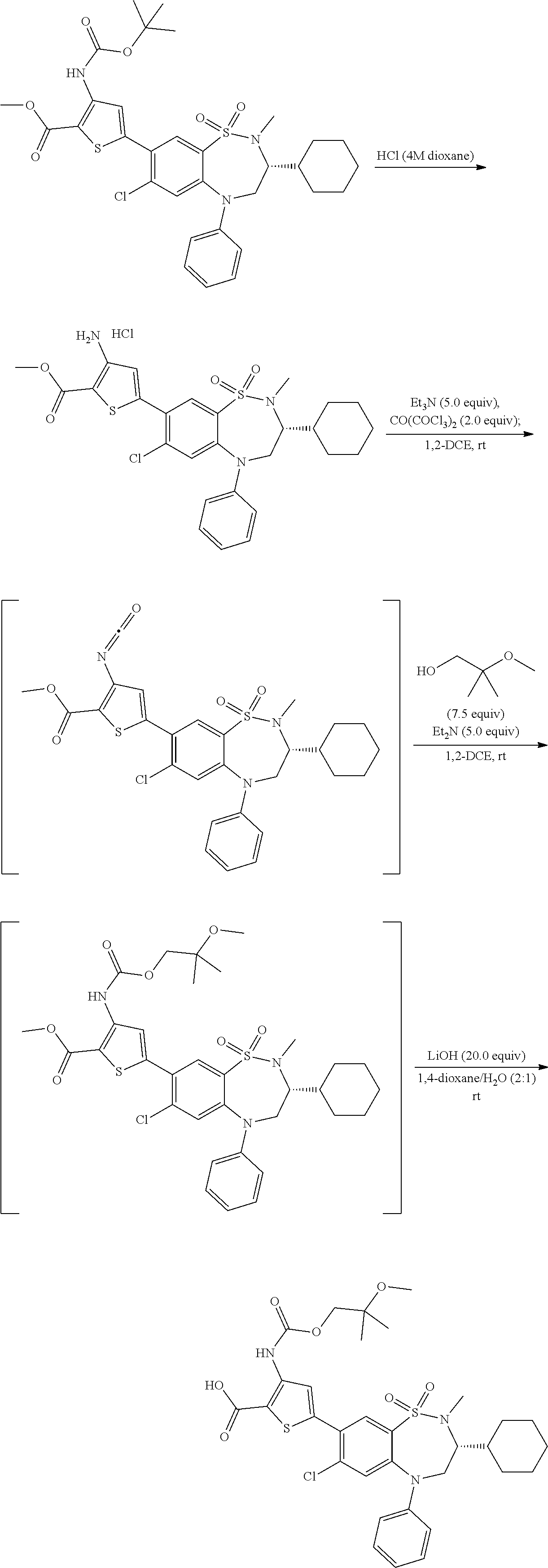

Step 1

In a 40 mL vial equipped with a stir bar, methyl (R)-3-((tert-butoxycarbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate (510 mg, 0.772 mmol, 1.0 equiv) was treated with 4M HCl in 1,4-dioxane (2.90 mL, 15.0 equiv). The reaction was stirred for 2 h at room temperature and concentrated to afford methyl (R)-3-amino-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate hydrochloride (461.0 mg, theoretical) which was used in the subsequent step without purification. ESI MS m/z=560.0 [M+H]⁺.

Step 2

In a 4 mL vial equipped with a stir bar, triphosgene (49.7 mg, 0.168 mmol, 2.0 equiv) was dissolved in 1,2-dichloroethane (0.5 mL). Next, triethylamine (42.4 mg, 58 μL, 5.0 equiv) was added. To the resulting solution was added (R)-3-amino-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate hydrochloride (50.0 mg, 0.084 mmol, 1.0 equiv) dropwise as a solution in 1,2-dichloroethane (1.0 mL). After 15 min, LCMS analysis indicated full conversion to methyl (R)-5-(7-chloro-3-cyclohexyl-2-methyl-1,1-dioxido-5-phenyl-2,3,4,5-tetrahydrobenzo[f][1,2,5]thiadiazepin-8-yl)-3-isocyanatothiophene-2-carboxylate which was used directly in the subsequent step.

Step 3

To the solution of methyl (R)-5-(7-chloro-3-cyclohexyl-2-methyl-1,1-dioxido-5-phenyl-2,3,4,5-tetrahydrobenzo[f][1,2,5]thiadiazepin-8-yl)-3-isocyanatothiophene-2-carboxylate formed in step 2 was added 2-methoxy-2-methylpropan-1-ol (65.5 mg, 0.629 mmol, 7.5 equiv, CAS #22665-67-4) as a solution in 1,2-dichloroethane (0.5 mL). Additional triethylamine (42.4 mg, 58 μL, 5.0 equiv) was then added. Within 1 h, LCMS analysis indicated full conversion to methyl (R)-5-(7-chloro-3-cyclohexyl-2-methyl-1,1-dioxido-dioxido-5-phenyl-2,3,4,5-tetrahydrobenzo[f][1,2,5]thiadiazepin-8-yl)-3-(((2-methoxy-2-methylpropoxy)carbonyl)amino)thiophene-2-carboxylate. The reaction mixture was then concentrated.

Step 4

The crude residue formed above was re-dissolved in 1,4-dioxane (1.0 mL) and water (0.5 mL) and lithium hydroxide (40.1 mg, 1.676 mmol, 20.0 equiv) was added and the mixture was stirred at room temperature. Upon full conversion, as judged by LCMS analysis, the reaction mixture was quenched with formic acid (0.25 mL) and passed through a 0.45 micron syringe filter using N,N-dimethylformamide to rinse before being purified by RPHPLC. The product obtained was lyophilized from acetonitrile and water to afford (R)-5-(7-chloro-3-cyclohexyl-2-methyl-1,1-dioxido-5-phenyl-2,3,4,5-tetrahydrobenzo[f][1,2,5]thiadiazepin-8-yl)-3-(((2-methoxy-2-methylpropoxy)carbonyl)amino)thiophene-2-carboxylic acid, Ex.239 (26.8 mg, 470 yield). ESI MS m/z=676.3 [M+H]⁺. ¹H NMR(400 MHz, CD3CN) 69.54 (s, 1H), 8.12 (s, 1H), 8.00 (s, 1H), 7.37 (dd, J=9.2, 6.5 Hz, 2H), 7.23-6.96 (m, 4H), 4.30 (d, J=16.0 Hz, 1H), 4.12 (s, 2H), 3.90 (s, 1H), 3.34 (t, J=10.7 Hz, 1H), 3.22 (s, 3H), 2.80 (s, 3H), 2.03 (d, J=13.1 Hz, 1H), 1.87 (d, J=12.1 Hz, 1H), 1.81-1.62 (m, 4H), 1311(1.36-1.13 (m, 9H), 1.06-0.97 (m,2H).

The following compounds were prepared using a procedure analogous to that described for Ex.239:

| Example | Structure | MS | NMR |
|---|---|---|---|
| 239 | 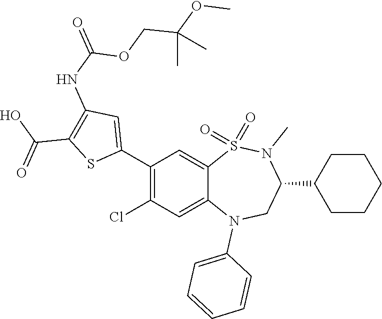 [SMILES: COC(C)(C)COC(=O)Nc1cc(-c2cc3c(cc2Cl)N(c2ccccc2)C[C@@H](C2CCCCC2)N(C)S3(=O)=O)sc1C(=O)O] | [M + H]⁺ 676.3 | ¹H NMR (400 MHz, CD₃CN) δ 9.54 (s, 1H), 8.12 (s, 1H), 8.00 (s, 1H), 7.37 (dd, J = 9.2, 6.5 Hz, 2H), 7.23-6.96 (m, 4H), 4.30 (d, J = 16.0 Hz, 1H), 4.12 (s, 2H), 3.90 (s, 1H), 3.34 (t, J = 10.7 Hz, 1H), 3.22 (s, 3H), 2.80 (s, 3H), 2.03 (d, J = 13.1 Hz, 1H), 1.87 (d, J = 12.1 Hz, 1H), 1.81-1.62 (m, 4H), 1.36-1.13 (m, 9H), 1.06-0.97 (m, 2H) |
| 240 | 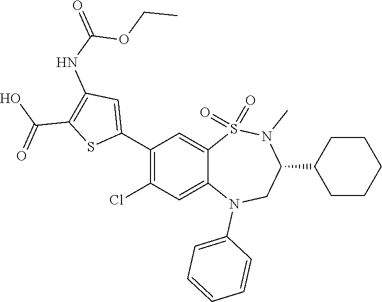 [SMILES: CCOC(=O)Nc1cc(-c2cc3c(cc2Cl)N(c2ccccc2)C[C@@H](C2CCCCC2)N(C)S3(=O)=O)sc1C(=O)O] | [M + H]⁺ 618.2 | |
| 241 | 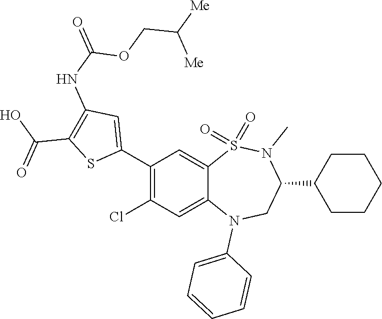 [SMILES: CC(C)COC(=O)Nc1cc(-c2cc3c(cc2Cl)N(c2ccccc2)C[C@@H](C2CCCCC2)N(C)S3(=O)=O)sc1C(=O)O] | [M + H]⁺ 646.2 | ¹H NMR (400 MHz, CD₃CN) δ 9.52 (s, 1H), 8.13 (s, 1H), 8.00 (s, 1H), 7.37 (t, J = 7.8 Hz, 2H), 7.16-7.06 (m, 4H), 4.30 (d, J = 16.0 Hz, 1H), 4.01- 3.99 (m, 2H), 3.90 (s, 1H), 3.37-3.34 (m, 1H), 2.79 (s, 3H). 2.06-1.99 (m, 2H), 1.88 (d, J = 12.6 Hz, 1H), 1.80- 1.64 (m, 4H), 1.34-1.14 (m, 3H), 1.07-0.97 (m, 8H). |
| 242 | 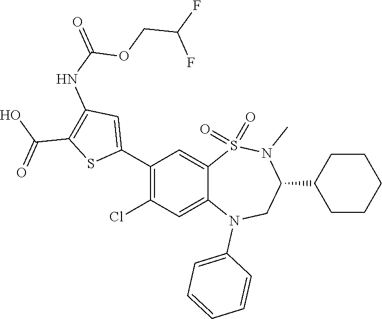 [SMILES: CN1[C@H](C2CCCCC2)CN(c2ccccc2)c2cc(Cl)c(-c3cc(NC(=O)OCC(F)F)c(C(=O)O)s3)cc2S1(=O)=O] | [M + H]⁺ 654.2 | |
| 243 | 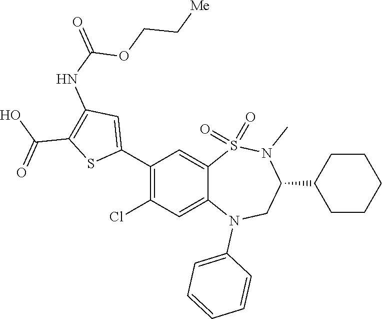 [SMILES: CCCOC(=O)Nc1cc(-c2cc3c(cc2Cl)N(c2ccccc2)C[C@@H](C2CCCCC2)N(C)S3(=O)=O)sc1C(=O)O] | [M + H]⁺ 632.1 | |
| 244 | 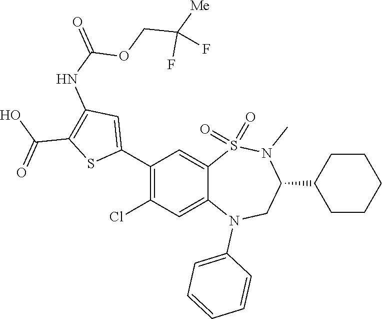 [SMILES: CN1[C@H](C2CCCCC2)CN(c2ccccc2)c2cc(Cl)c(-c3cc(NC(=O)OCC(C)(F)F)c(C(=O)O)s3)cc2S1(=O)=O] | [M + H]⁺ 668.3 | |
| 245 | 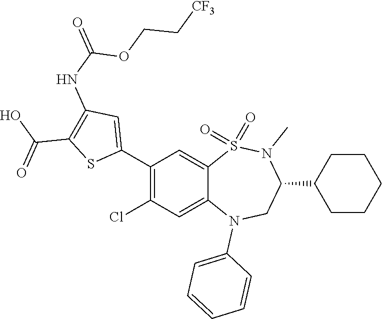 [SMILES: CN1[C@H](C2CCCCC2)CN(c2ccccc2)c2cc(Cl)c(-c3cc(NC(=O)OCCC(F)(F)F)c(C(=O)O)s3)cc2S1(=O)=O] | [M + H]⁺ 686.2 | |
| 246 | 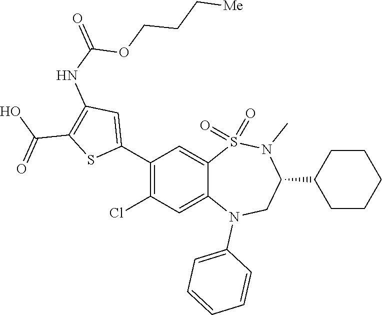 [SMILES: CCCCOC(=O)Nc1cc(-c2cc3c(cc2Cl)N(c2ccccc2)C[C@@H](C2CCCCC2)N(C)S3(=O)=O)sc1C(=O)O] | [M + H]⁺ 646.7 | |
| 247 | 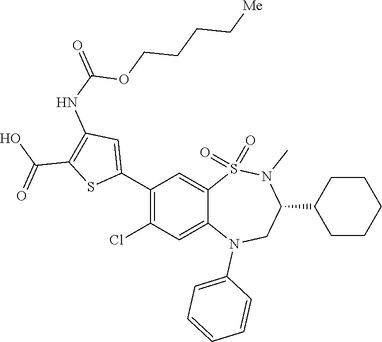 [SMILES: CCCCCOC(=O)Nc1cc(-c2cc3c(cc2Cl)N(c2ccccc2)C[C@@H](C2CCCCC2)N(C)S3(=O)=O)sc1C(=O)O] | [M + H]⁺ 660.4 | |
| 248 | 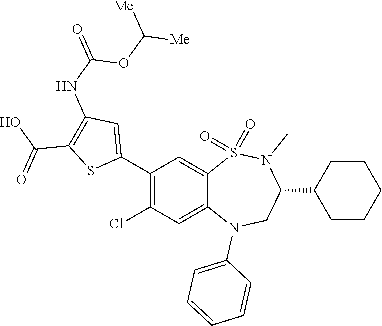 [SMILES: CC(C)OC(=O)Nc1cc(-c2cc3c(cc2Cl)N(c2ccccc2)C[C@@H](C2CCCCC2)N(C)S3(=O)=O)sc1C(=O)O] | [M + H]⁺ 632.3 | |
| 249 | 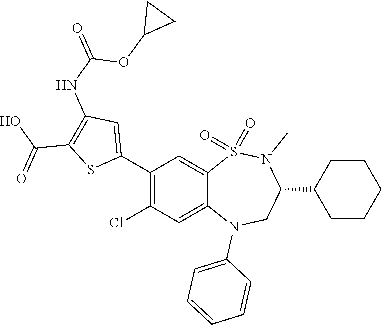 [SMILES: CN1[C@H](C2CCCCC2)CN(c2ccccc2)c2cc(Cl)c(-c3cc(NC(=O)OC4CC4)c(C(=O)O)s3)cc2S1(=O)=O] | [M + H]⁺ 630.3 | |
| 250 | 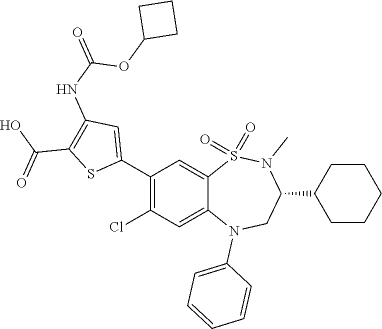 [SMILES: CN1[C@H](C2CCCCC2)CN(c2ccccc2)c2cc(Cl)c(-c3cc(NC(=O)OC4CCC4)c(C(=O)O)s3)cc2S1(=O)=O] | [M + H]⁺ 644.3 | |
| 251 | 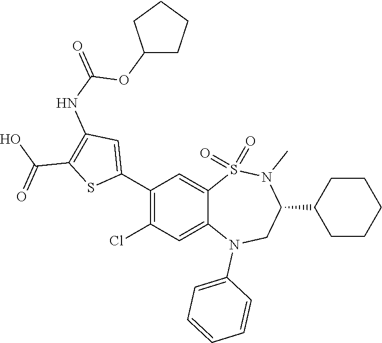 [SMILES: CN1[C@H](C2CCCCC2)CN(c2ccccc2)c2cc(Cl)c(-c3cc(NC(=O)OC4CCCC4)c(C(=O)O)s3)cc2S1(=O)=O] | [M + H]⁺ 658.3 | |
| 252 | 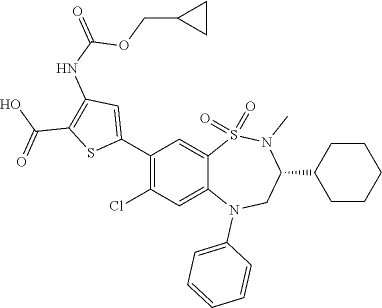 [SMILES: CN1[C@H](C2CCCCC2)CN(c2ccccc2)c2cc(Cl)c(-c3cc(NC(=O)OCC4CC4)c(C(=O)O)s3)cc2S1(=O)=O] | [M + H]⁺ 644.1 | ¹H NMR (500 MHz, CDCl₃) δ 9.39 (s, 1H), 8.17 (s, 1H), 8.07 (s, 1H), 7.39 (t, J = 7.7 Hz, 2H), 7.19-7.07 (m, 3H), 6.99 (s, 1H), 4.26 (s, 2H), 4.03 (d, J = 7.3 Hz, 2H), 3.16 (s, 1H), 2.92 (s, 3H), 2.17 (d, J = 12.8 Hz, 1H), 1.91-1.58 (m, 6H), 1.37-1.18 (m, 3H), 1.11 (q, J = 12.8 Hz, 1H), 0.92 (dd, J = 18.4, 6.8 Hz, 2H), 0.62 (q, J = 5.7 Hz, 2H), 0.35 (t, J = 5.1 Hz, 2H). |
| 253 | 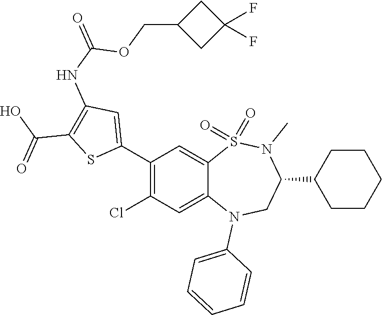 [SMILES: CN1[C@H](C2CCCCC2)CN(c2ccccc2)c2cc(Cl)c(-c3cc(NC(=O)OCC4CC(F)(F)C4)c(C(=O)O)s3)cc2S1(=O)=O] | [M + H]⁺ 694.2 | |
| 254 | 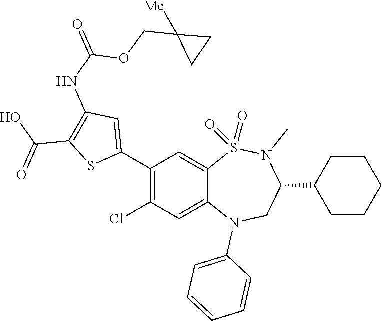 [SMILES: CN1[C@H](C2CCCCC2)CN(c2ccccc2)c2cc(Cl)c(-c3cc(NC(=O)OCC4(C)CC4)c(C(=O)O)s3)cc2S1(=O)=O] | [M + H]⁺ 660.2 | ¹H NMR (500 MHz, CDCl₃) δ 9.39 (s, 1H), 8.17 (s, 1H), 8.07 (s, 1H), 7.39 (t, J = 7.7 Hz, 2H), 7.18-7.10 (m, 3H), 6.99 (s, 1H), 4.26 (s, 2H), 4.01 (s, 2H), 3.16 (s, 1H), 2.92 (s, 3H), 2.17 (d, J = 12.9 Hz, 1H), 1.86-1.61 (m, 5H), 1.34-1.21 (m, 2H), 1.20 (s, 3H), 1.16- 1.06 (m, 1H), 0.92 (h, J = 11.5 Hz, 2H), 0.58-0.52 (m, 2H), 0.45-0.38 (m, 2H) |
| 255 | 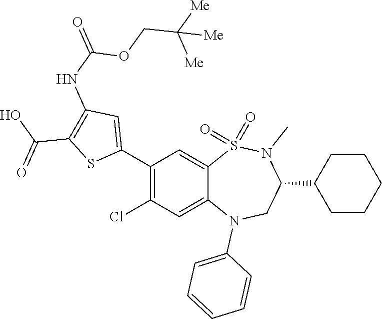 [SMILES: CN1[C@H](C2CCCCC2)CN(c2ccccc2)c2cc(Cl)c(-c3cc(NC(=O)OCC(C)(C)C)c(C(=O)O)s3)cc2S1(=O)=O] | [M + H]⁺ 660.4 | |
| 256 | 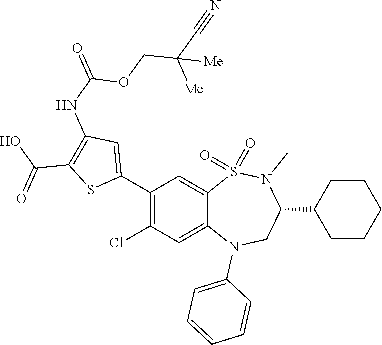 [SMILES: CN1[C@H](C2CCCCC2)CN(c2ccccc2)c2cc(Cl)c(-c3cc(NC(=O)OCC(C)(C)C#N)c(C(=O)O)s3)cc2S1(=O)=O] | [M + H]⁺ 671.1 | 1H NMR (400 MHz, CD3CN) δ 9.69 (s, 1H), 8.11 (s, 1H), 8.01 (s, 1H), 7.38 (t, J = 7.8 Hz, 2H), 7.21-7.01 (m, 4H), 4.30 (d, J = 16.1 Hz, 1H), 4.22 (s, 2H), 3.90 (s, 1H), 3.34 (t, J = 10.7 Hz, 1H), 2.80 (s, 3H), 1.78-1.73 (m, 3H), 1.70-1.65 (m, 2H), 1.43 (s, 6H), 1.33- 1.16 (m, 4H), 1.07-0.97 (m, 2H) |
| 257 | 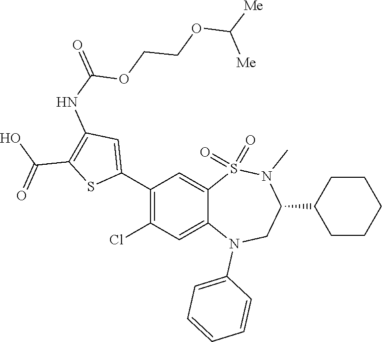 [SMILES: CC(C)OCCOC(=O)Nc1cc(-c2cc3c(cc2Cl)N(c2ccccc2)C[C@@H](C2CCCCC2)N(C)S3(=O)=O)sc1C(=O)O] | [M + H]⁺ 676.0 | |
| 258 | 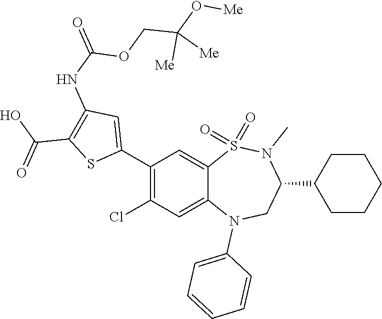 [SMILES: COC(C)(C)COC(=O)Nc1cc(-c2cc3c(cc2Cl)N(c2ccccc2)C[C@@H](C2CCCCC2)N(C)S3(=O)=O)sc1C(=O)O] | [M + H]⁺ 676.2 | |
| 259 | 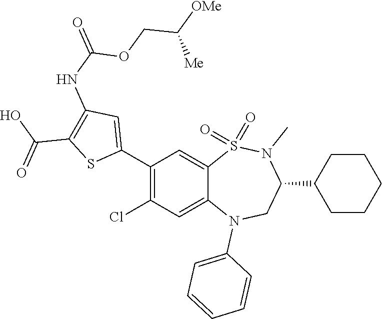 [SMILES: CO[C@H](C)COC(=O)Nc1cc(-c2cc3c(cc2Cl)N(c2ccccc2)C[C@@H](C2CCCCC2)N(C)S3(=O)=O)sc1C(=O)O] | [M + H]⁺ 662.2 | |
| 260 | 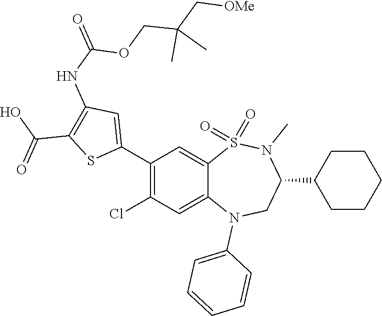 [SMILES: COCC(C)(C)COC(=O)Nc1cc(-c2cc3c(cc2Cl)N(c2ccccc2)C[C@@H](C2CCCCC2)N(C)S3(=O)=O)sc1C(=O)O] | [M + H]⁺ 690.2 | |
| 261 | 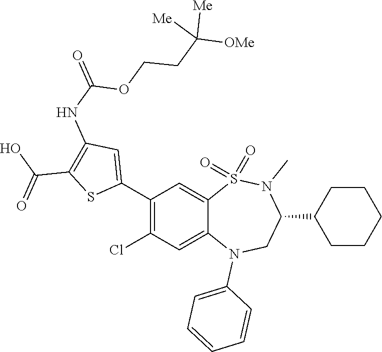 [SMILES: COC(C)(C)CCOC(=O)Nc1cc(-c2cc3c(cc2Cl)N(c2ccccc2)C[C@@H](C2CCCCC2)N(C)S3(=O)=O)sc1C(=O)O] | [M + H]⁺ 690.1 | |
| 262 | 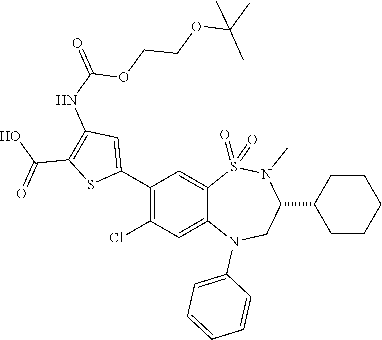 [SMILES: CN1[C@H](C2CCCCC2)CN(c2ccccc2)c2cc(Cl)c(-c3cc(NC(=O)OCCOC(C)(C)C)c(C(=O)O)s3)cc2S1(=O)=O] | [M − C₄H₈ + H]⁺ 634.2 | |
| 263 | 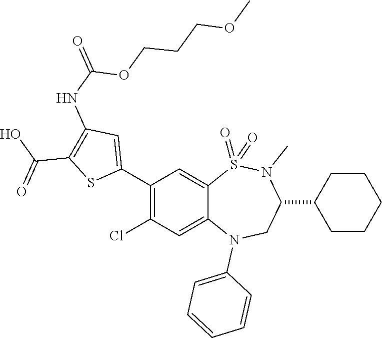 [SMILES: COCCCOC(=O)Nc1cc(-c2cc3c(cc2Cl)N(c2ccccc2)C[C@@H](C2CCCCC2)N(C)S3(=O)=O)sc1C(=O)O] | [M + H]⁺ 662.3 | |
| 264 | 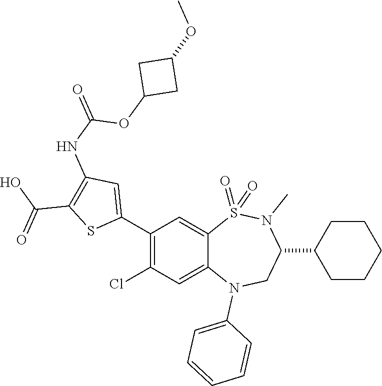 | [M + H]⁺ 674.0 | |

### Ex.265: Synthesis of(R)-5-(3-cyclohexyl-7-fluoro-2-methyl-1,1-dioxido-5-phenyl-2,3,4,5-tetrahydrobenzo[f][1,2,5]thiadiazepin-8-yl)-3-((ethoxycarbonyl)amino)thiophene-2-carboxylic acid

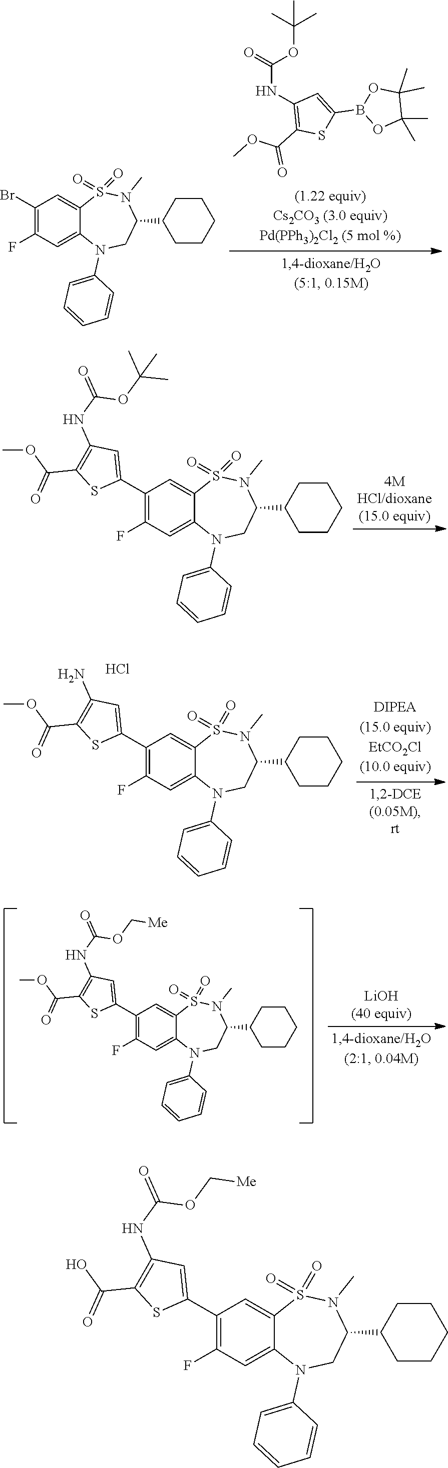

Step 1

In a 40 mL vial equipped with a stir bar, (R)-8-bromo-3-cyclohexyl-7-fluoro-2-methyl-5-phenyl-2,3,4,5-tetrahydrobenzo[f][1,2,5]thiadiazepine 1,1-dioxide (800 mg, 1.71 mmol, 1.0 equiv) was combined neat with methyl 3-((tert-butoxycarbonyl)amino)-5-(4,4,5,5-tetramethyl-1,3,2-dioxaborolan-2-yl)thiophene-2-carboxylate (800 mg, 2.09 mmol, 1.22 equiv, CAS #2377606-49-8), cesium carbonate (1.67 g, 5.13 mmol, 3.0 equiv), and bis(triphenylphosphine)palladium(II) chloride (60.1 mg, 0.086 mmol, 5 mol %, CAS #13965-03-2) under a nitrogen atmosphere. Next, 1,4-dioxane (9.5 mL) and water (1.9 mL) were added. The vial was then sealed with electrical tape and heated at 80° C. for 2 h. Upon cooling to room temperature, the mixture was concentrated and purified by silica gel column chromatography (cyclohexane/ethyl acetate) to afford methyl (R)-3-((tert-butoxycarbonyl)amino)-5-(3-cyclohexyl-7-fluoro-2-methyl-1,1-dioxido-5-phenyl-2,3,4,5-tetrahydrobenzo[f][1,2,5]thiadiazepin-8-yl)thiophene-2-carboxylate (684.0 mg, 62% yield). ESI MS m/z=544.1 [M-C₄H₈-CO₂+H]⁺.

Step 2

In a 40 mL vial equipped with a stir bar, methyl (R)-3-((tert-butoxycarbonyl)amino)-5-(3-cyclohexyl-7-fluoro-2-methyl-1,1-dioxido-5-phenyl-2,3,4,5-tetrahydrobenzo[f][1,2,5]thiadiazepin-8-yl)thiophene-2-carboxylate (684 mg, 1.06 mmol, 1.0 equiv) was charged with 4M HCl in 1,4-dioxane (3.98 mL, 15.0 equiv). The mixture was stirred for 1.5 h and concentrated to afford methyl (R)-3-amino-5-(3-cyclohexyl-7-fluoro-2-methyl-1,1-dioxido-5-phenyl-2,3,4,5-tetrahydrobenzo[f][1,2,5]thiadiazepin-8-yl)thiophene-2-carboxylate hydrochloride that was used in the next step without purification (616 mg, theoretical). ESI MS m/z=544.0 [M+H]⁺.

Step 3

Methyl (R)-3-amino-5-(3-cyclohexyl-7-fluoro-2-methyl-1,1-dioxido-5-phenyl-2,3,4,5-tetrahydrobenzo[f][1,2,5]thiadiazepin-8-yl)thiophene-2-carboxylate hydrochloride was suspended in 1,2-dichloroethane (0.84 mL). Next, N,N-diisopropylethylamine (0.11 mL) was added, followed by ethyl chloroformate (45.5 mg, 40 μL). The resulting mixture was stirred for 24 h. At this time, LCMS analysis indicated conversion to methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-((ethoxycarbonyl)amino)thiophene-2-carboxylate, and the reaction mixture was concentrated.

Step 4

The residue formed above in step 3 was dissolved in a mixture of 1,4-dioxane (0.75 mL) and water (0.25 mL) and lithium hydroxide (40.1 mg) was added. The mixture was heated at 50° C. for 3 h. Upon cooling to room temperature, the reaction mixture was quenched with formic acid (0.25 mL) and passed through a 0.45 micron syringe filter using N,N-dimethylformamide to rinse before being purified by RPHPLC. Lyophilization of the isolated product gave (R)-5-(3-cyclohexyl-7-fluoro-2-methyl-1,1-dioxido-5-phenyl-2,3,4,5-tetrahydrobenzo[f][1,2,5]thiadiazepin-8-yl)-3-((ethoxycarbonyl)amino)thiophene-2-carboxylic acid as a white solid, Ex.265 (1.52 mg, 6%). ESI MS m/z=602.2 [M+H]⁺.

The following compounds were prepared using a procedure analogous to that described for Ex.265:

| Example | Structure | MS | NMR |
|---|---|---|---|
| 265 | 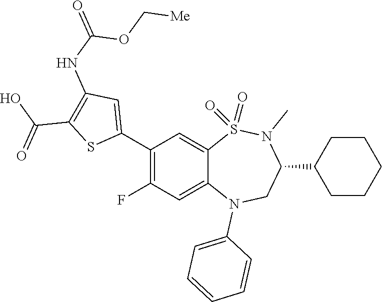 [SMILES: CCOC(=O)Nc1cc(-c2cc3c(cc2F)N(c2ccccc2)C[C@@H](C2CCCCC2)N(C)S3(=O)=O)sc1C(=O)O] | [M + H]⁺ 602.2 | |
| 266 | 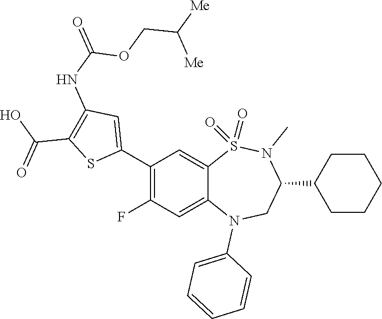 [SMILES: CC(C)COC(=O)Nc1cc(-c2cc3c(cc2F)N(c2ccccc2)C[C@@H](C2CCCCC2)N(C)S3(=O)=O)sc1C(=O)O] | [M + H]⁺ 630.3 | ¹H NMR (400 MHz, CD₃CN) δ 9.52 (s, 1H), 8.20 (s, 1H), 8.14 (d, J = 8.4 Hz, 1H), 7.38 (t, J = 7.8 Hz, 2H), 7.13-7.09 (m, 3H), 6.80 (d, J = 13.2 Hz, 1H), 4.29 (d, J = 15.9 Hz, 1H), 4.01 (d, J = 6.6 Hz, 2H), 3.35-3.30 (m, 1H), 2.82 (s, 3H), 1.87-1.84 (m, 1H), 1.80-1.66 (m, 4H), 1.33-1.15 (m, 4H), 1.07-0.95 (m, 8H). |

### Ex.267: Synthesis of(R)-5-(7-chloro-3-cyclohexyl-2-methyl-1,1-dioxido-5-phenyl-2,3,4,5-tetrahydrobenzo[f][1,2,5]thiadiazepin-8-yl)-2-((2-methylpropyl)sulfonamido)benzoic acid

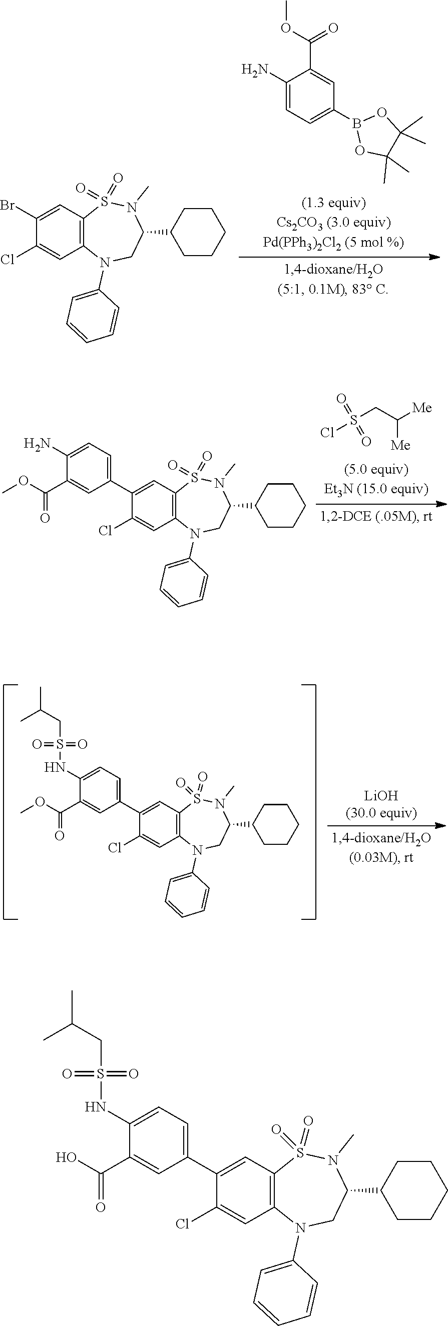

Step 1

In a 40 mL vial equipped with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (513.5 mg, 1.06 mmol, 1.0 equiv) was combined neat with methyl 2-amino-5-(4,4,5,5-tetramethyl-1,3,2-dioxaborolan-2-yl)benzoate (382.0 mg, 1.38 mmol, 1.3 equiv, CAS #363185-87-9), cesium carbonate (1.04 g, 3.18 mmol, 3.0 equiv), and bis(triphenylphosphine)palladium(II) chloride (37.2 mg, 0.053 mmol, 5 mol %) under a nitrogen atmosphere. Next, 1,4-dioxane (8.8 mL) and water (1.8 mL) were added, and the vial was sealed with electrical tape. The reaction was heated at 83° C. for 2 h. Upon cooling to room temperature, the mixture was concentrated and purified by silica gel column chromatography (0 to 40% ethyl acetate/cyclohexane) to afford methyl (R)-2-amino-5-(7-chloro-3-cyclohexyl-2-methyl-1,1-dioxido-5-phenyl-2,3,4,5-tetrahydrobenzo[f][1,2,5]thiadiazepin-8-yl)benzoate as an off white solid (482.5 mg, 82%). ESI MS m/z=554.0 [M+H]⁺.

Step 2

In a 1 mL vial equipped with a stir bar, methyl (R)-2-amino-5-(7-chloro-3-cyclohexyl-2-methyl-1,1-dioxido-5-phenyl-2,3,4,5-tetrahydrobenzo[f][1,2,5]thiadiazepin-8-yl)benzoate (15.0 mg, 0.027 mmol, 1.0 equiv) was dissolved in 1,2-dichloroethane (0.5 mL, 0.05M). Next, triethylamine (41.1 mg, 57 μL, 0.406 mmol, 15.0 equiv) was added, followed by 2-methylpropane-1-sulfonyl chloride (21.2 mg, 0.135 mmol, 5.0 equiv, CAS #35432-36-1). The resulting mixture was stirred at room temperature until LCMS analysis indicated complete consumption of the starting material. Concentration afforded a crude residue which was used directly.

Step 3

The residue obtained in step 2 was dissolved in 1,4-dioxane (0.7 mL) and water (0.3 mL). Lithium hydroxide (19.5 mg, 0.81 mmol, 30.0 equiv) was added, and the mixture was stirred at room temperature until LCMS analysis indicated full consumption of the starting material. The reaction mixture was quenched with formic acid (0.25 mL) and purified by RPHPLC. Upon isolation, the product was lyophilized from a mixture of water and acetonitrile to afford (R)-5-(7-chloro-3-cyclohexyl-2-methyl-1,1-dioxido-5-phenyl-2,3,4,5-tetrahydrobenzo[f][1,2,5]thiadiazepin-8-yl)-2-((2-methylpropyl)sulfonamido)benzoic acid, Ex.267 as a white solid (3.4 mg, 19% yield). ESI MS m/z=660.2 [M+H]⁺. ¹H NMR (400 MHz, CD₃CN) δ 10.61 (s, 1H), 8.19 (d, J=2.0 Hz, 1H), 7.83 (s, 1H), 7.81-7.71 (m, 2H), 7.39-7.24 (m, 3H), 7.00 (t, J=7.3 Hz, 3H), 4.35 (d, J=15.9 Hz, 1H), 3.68 (s, 1H), 3.42 (t, J=10.6 Hz, 1H), 3.20 (d, J=6.5 Hz, 2H), 2.72 (s, 3H), 1.97 (m, 2H), 1.80-1.76 (m, 2H), 1.69-1.61 (m, 2H), 1.36-1.18 (m, 4H), 1.13-0.98 (m, 8H).

The following compounds were prepared using a procedure analogous to that used to prepare Ex.267:

| Example | Structure | MS | NMR |
|---|---|---|---|
| 267 | 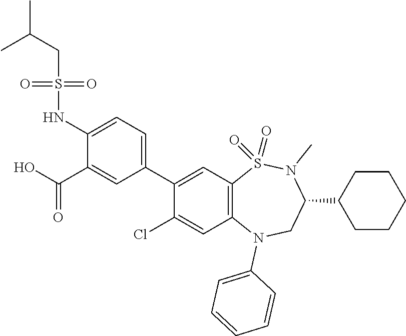 | [M + H]⁺ 554.0 | ¹H NMR (400 MHz, CD₃CN) δ 10.61 (s, 1H), 8.19 (d, J = 2.0 Hz, 1H), 7.83 (s, 1H), 7.81 − 7.71 (m, 2H), 7.39 − 7.24 (m, 3H), 7.00 (t, J = 7.3 Hz, 3H), 4.35 (d, J = 15.9 Hz, 1H), 3.68 (s, 1H), 3.42 (t, J = 10.6 Hz, 1H), 3.20 (d, J = 6.5 Hz, 2H), 2.72 (s, 3H), 1.97 (m, 2H), 1.80 − 1.76 (m, 2H), 1.69 − 1.61 (m, 2H), 1.36 − 1.18 (m, 4H), 1.13 − 0.98 (m, 8H) |
| 268 | 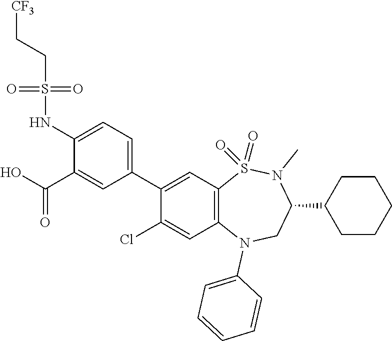 [SMILES: CN1[C@H](C2CCCCC2)CN(c2ccccc2)c2cc(Cl)c(-c3ccc(NS(=O)(=O)CCC(F)(F)F)c(C(=O)O)c3)cc2S1(=O)=O] | [M + H]⁺ 700.1 | |
| 269 | 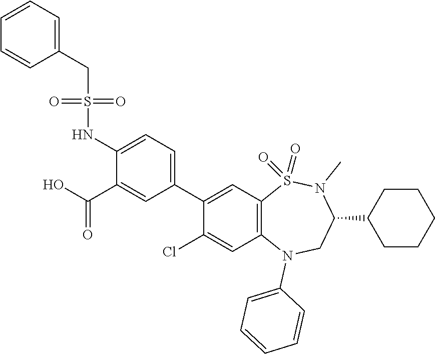 [SMILES: CN1[C@H](C2CCCCC2)CN(c2ccccc2)c2cc(Cl)c(-c3ccc(NS(=O)(=O)Cc4ccccc4)c(C(=O)O)c3)cc2S1(=O)=O] | [M + H]⁺ 694.1 | |
| 270 | 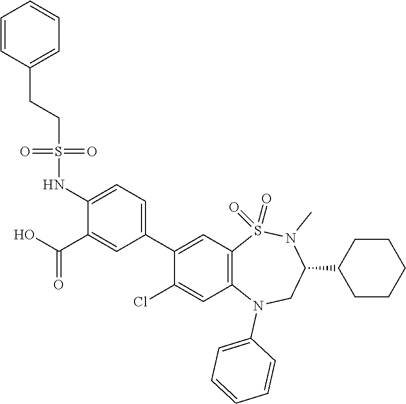 [SMILES: CN1[C@H](C2CCCCC2)CN(c2ccccc2)c2cc(Cl)c(-c3ccc(NS(=O)(=O)CCc4ccccc4)c(C(=O)O)c3)cc2S1(=O)=O] | [M + H]⁺ 708.4 | |
| 271 | 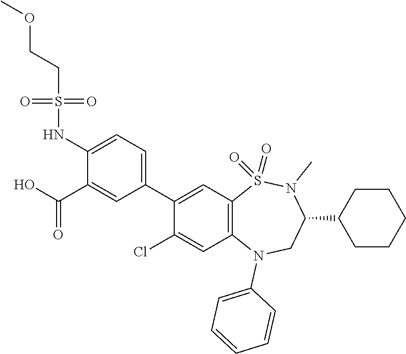 [SMILES: COCCS(=O)(=O)Nc1ccc(-c2cc3c(cc2Cl)N(c2ccccc2)C[C@@H](C2CCCCC2)N(C)S3(=O)=O)cc1C(=O)O] | [M + H]⁺ 663.3 | |
| 272 | 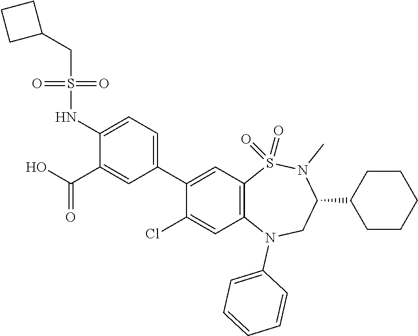 [SMILES: CN1[C@H](C2CCCCC2)CN(c2ccccc2)c2cc(Cl)c(-c3ccc(NS(=O)(=O)CC4CCC4)c(C(=O)O)c3)cc2S1(=O)=O] | [M + H]⁺ 672.1 | |
| 273 | 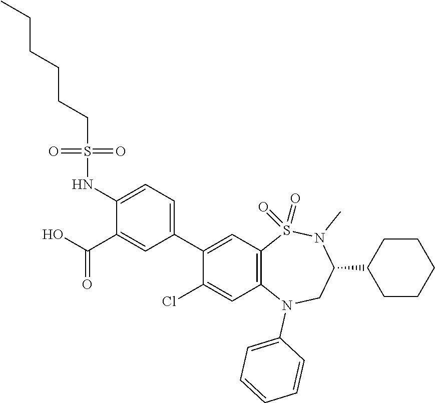 [SMILES: CCCCCCS(=O)(=O)Nc1ccc(-c2cc3c(cc2Cl)N(c2ccccc2)C[C@@H](C2CCCCC2)N(C)S3(=O)=O)cc1C(=O)O] | [M + H]⁺ 688.1 | |
| 274 | 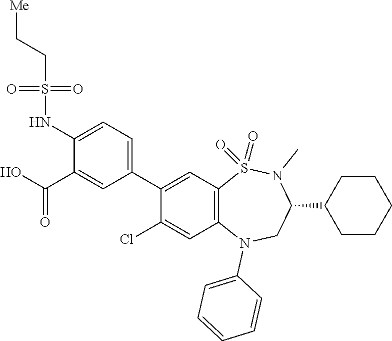 [SMILES: CCCS(=O)(=O)Nc1ccc(-c2cc3c(cc2Cl)N(c2ccccc2)C[C@@H](C2CCCCC2)N(C)S3(=O)=O)cc1C(=O)O] | [M + H]⁺ 646.1 | |

### Ex.275: Synthesis of (R)-5-(7-chloro-3-cyclohexyl-2-methyl-1,1-dioxido-5-phenyl-2,3,4,5-tetrahydrobenzo[f][1,2,5]thiadiazepin-8-yl)-3-(propylsulfonamido)thiophene-2-carboxylic acid

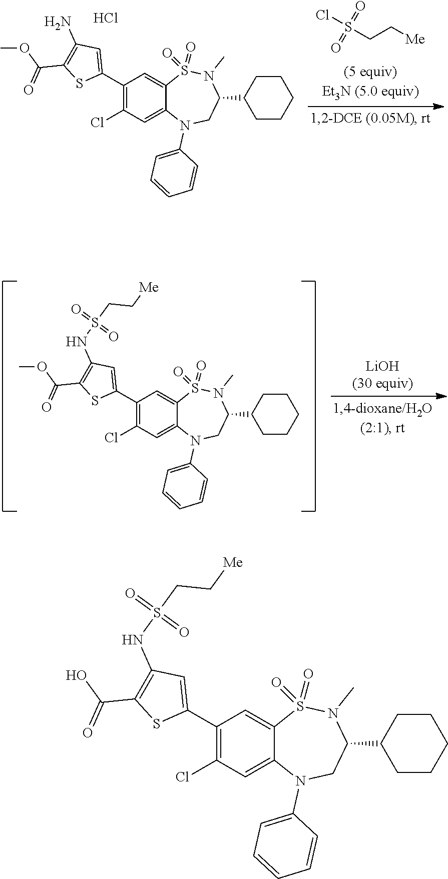

Step 1

In 1 4 mL vial equipped with a stir bar, methyl (R)-3-amino-5-(7-chloro-3-cyclohexyl-2-methyl-1,1-dioxido-5-phenyl-2,3,4,5-tetrahydrobenzo[f][1,2,5]thiadiazepin-8-yl)thiophene-2-carboxylate hydrochloride (25.0 mg, 0.04 mmol, 1.0 equiv) was dissolved in 1,2-dichloroethane (0.84 mL).

Triethylamine (63.6 mg, 88 μL, 0.63 mmol, 15.0 equiv) was then added, followed by propane-1-sulfonyl chloride (29.9 mg, 0.210 mmol, 5.0 equiv, CAS#10147-36-1). The resulting mixture was stirred for 2 h and concentrated to afford a crude residue which was used directly in the next step.

Step 2

The crude residue generated above in step 1 was dissolved in a mixture of 1,4-dioxane (0.5 mL) and water (0.25 mL) and lithium hydroxide (30.1 mg, 1.26 mmol, 30 equiv) was added. The reaction mixture was then heated at 50° C. for 3 h. Additional lithium hydroxide (30.1 mg, 1.26 mmol, 30 equiv) was added, and the mixture was heated at 55° C. for 18 h. Upon cooling to room temperature, the reaction was quenched by the addition of formic acid (0.25 mL) and the mixture was purified by RPHPLC. The isolated product was lyophilized from a mixture of acetonitrile and water to afford (R)-5-(7-chloro-3-cyclohexyl-2-methyl-1,1-dioxido-5-phenyl-2,3,4,5-tetrahydrobenzo[f][1,2,5]thiadiazepin-8-yl)-3-(propylsulfonamido)thiophene-2-carboxylic acid, Ex.275 (13.1 mg, 48%). ESI MS m/z=646.2 [M+H]⁺.

Example 276 was prepared using a procedure analogous to that used to prepare Ex.275:

| Example | Structure | MS |
|---|---|---|
| 275 | 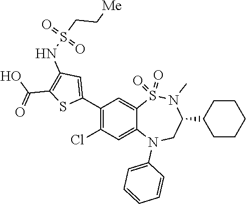 | [M + H]⁺ 646.2 |
| 276 | 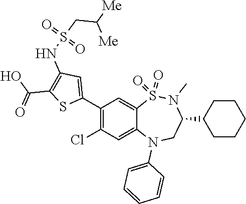 [SMILES: CC(C)CS(=O)(=O)Nc1cc(-c2cc3c(cc2Cl)N(c2ccccc2)C[C@@H](C2CCCCC2)N(C)S3(=O)=O)sc1C(=O)O] | [M + H]⁺ 666.1 |

### Ex.277: Synthesis of(R)-2-(((benzyloxy)carbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)-3-fluorobenzoic acid

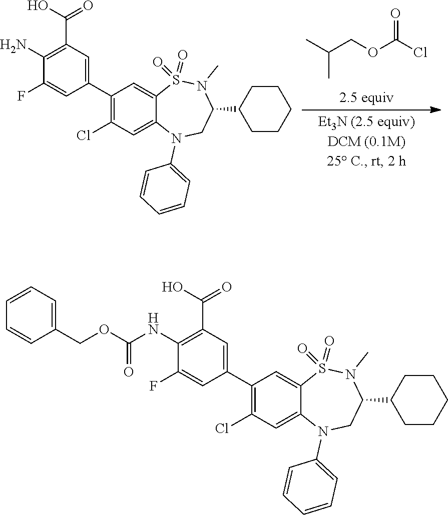

In a 4 mL vial equipped with a stir bar, (R)-2-amino-5-(7-chloro-3-cyclohexyl-2-methyl-1,1-dioxido-5-phenyl-2,3,4,5-tetrahydrobenzo[f][1,2,5]thiadiazepin-8-yl)-3-fluorobenzoic acid (30.0 mg, 1.0 equiv.) was dissolved in dry DCM (0.54 mL) and followed by addition of isobutyl chloroformate (17 μl, 2.5 equiv., CAS #543-27-1) and triethylamine (18 μl, 2.5 equiv.) at 0° C. Reaction mixture was stirred for 5 minutes at 0° C. and then ice bath was removed and continue stirring at 25° C. for 2 h. Then, mixture was concentrated under vacuum and the crude residue was re-dissolved in 4.0 mL of dimethyl sulfoxide and loaded in a 100 g C18 gold column for reversed phase flash chromatography purification to afford (R)-2-(((benzyloxy)carbonyl)amino)-5-(7-chloro-3-cyclohexyl-2-methyl-1,1-dioxido-5-phenyl-2,3,4,5-tetrahydrobenzo[f][1,2,5]thiadiazepin-8-yl)-3-fluorobenzoic acid (5 mg, 14% yield). ESI MS m/z=658.3 [M+H]⁺.

| Example | Structure | MS |
|---|---|---|
| 277 | 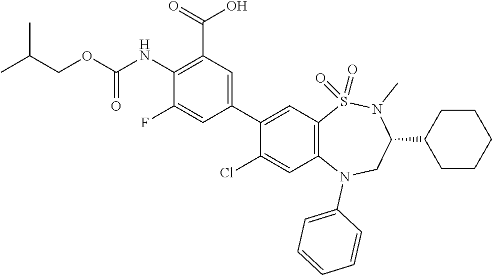 [SMILES: CC(C)COC(=O)Nc1c(F)cc(-c2cc3c(cc2Cl)N(c2ccccc2)C[C@@H](C2CCCCC2)N(C)S3(=O)=O)cc1C(=O)O] | [M + H]⁺ 658.3 |

The following compounds were prepared using procedures analogous to that described for Ex.277:

| Example | Structure | MS | NMR |
|---|---|---|---|
| 278 | 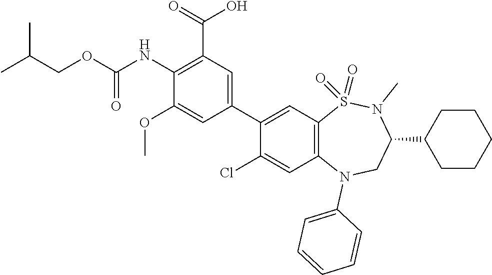 [SMILES: COc1cc(-c2cc3c(cc2Cl)N(c2ccccc2)C[C@@H](C2CCCCC2)N(C)S3(=O)=O)cc(C(=O)O)c1NC(=O)OCC(C)C] | [M + H]⁺ 670.3 | ¹H NMR (400 MHz, DMSO- d6) δ 8.83 (br, 1H), 7.77 (s, 1H), 7.43 − 7.16 (m, 5H), 7.07 − 6.87 (m, 3H), 4.47 − 4.29 (m, 1H), 3.85 (s, 3H), 3.80 (d, J = 6.7 Hz, 2H), 2.69 (s, 3H), 2.00 − 1.79 (m, 3H), 1.77 − 1.52 (m, 4H), 1.29 − 0.92 (m, 5H), 0.90 (d, J = 6.7 Hz, 6H). |
| 279 | 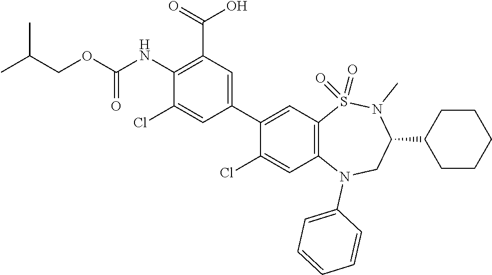 [SMILES: CC(C)COC(=O)Nc1c(Cl)cc(-c2cc3c(cc2Cl)N(c2ccccc2)C[C@@H](C2CCCCC2)N(C)S3(=O)=O)cc1C(=O)O] | [M + H]⁺ 674.2 | |

### Ex.280: Synthesis of(R)-5-(7-chloro-3-cyclohexyl-5-(5-fluoropyridin-3-yl)-2-methyl-1,1-dioxido-2,3,4,5-tetrahydrobenzo[f][1,2,5]thiadiazepin-8-yl)-2-fluorobenzoic acid

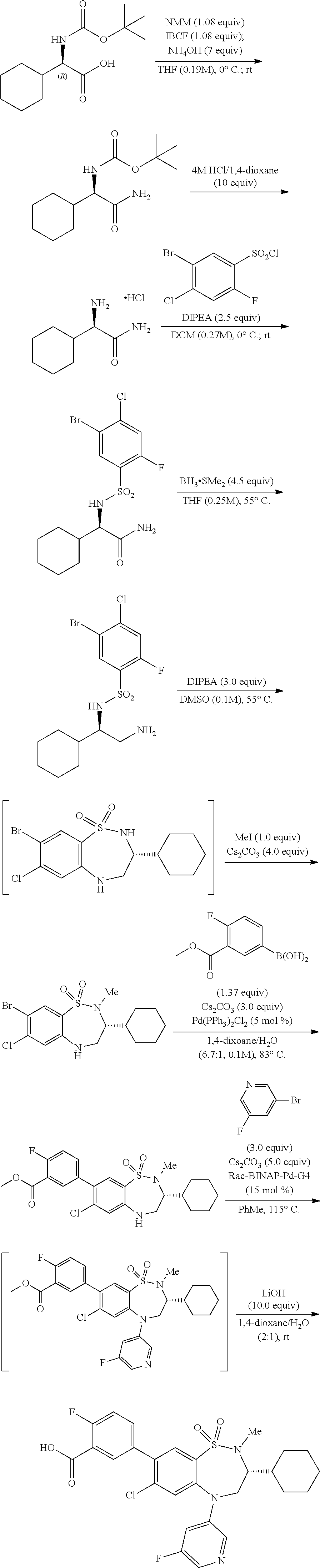

Step 1

In a 500 mL round bottom flask equipped with a stir bar, (R)-2-((tert-butoxycarbonyl)amino)-2-cyclohexylacetic acid (5.0 gram, 19.4 mmol, 1.0 equiv) was dissolved in tetrahydrofuran (100 mL, 0.19M). The resulting solution was cooled in an ice bath, and N-methylmorpholine (2.12 g, 2.31 mL, 20.98 mmol, 1.08 equiv) was added followed by dropwise addition of isobutyl chloroformate (2.87 g, 2.76 mL, 20.98 mmol, 1.08 equiv) over the course of 5 minutes. The resulting mixture was stirred for 15 minutes before ammonium hydroxide (15.9 g, 17.7 mL, 30 wt %, 7 equiv) was added. The reaction was stirred overnight while being allowed to warm to room temperature. Upon completion, 1.2M aqueous hydrochloric acid (200 mL) was added and the aqueous phase was extracted with ethyl acetate. The combined organic layers were dried over magnesium sulfate and concentrated to afford tert-butyl (R)-(2-amino-1-cyclohexyl-2-oxoethyl)carbamate (2.78 g, 56% yield). ESI MS m/z=157.2 [M-C₄Hs-CO₂+H]⁺.

Step 2

The tert-butyl (R)-(2-amino-1-cyclohexyl-2-oxoethyl)carbamate (2.78 g, 10.84 mmol, 1.0 equiv) produced above in step 1 was treated with 4M HCl in 1,4-dioxane (27.1 mL, 10.0 equiv). The resulting mixture was stirred for 40 min and additional 4M HCl in 1,4-dioxane (20 mL) was added. After 45 min, LCMS analysis indicated full consumption of the starting material, and the reaction was concentrated to afford (R)-2-amino-2-cyclohexylacetamide hydrochloride (2.09 g theoretical) that was used directly in the subsequent step without purification. ESI MS m/z=157.3 [M+H]⁺.

Step 3

The (R)-2-amino-2-cyclohexylacetamide hydrochloride (2.09 g theoretical) generated in step 2 above was suspended in dichloromethane (40 mL, 0.27M). The suspension was cooled in an ice bath, and N,N-diisopropylethylamine (3.50 g, 4.74 mL, 27.1 mmol, 2.5 equiv) was added. To the resulting homogenous solution was added 5-bromo-4-chloro-2-fluorobenzenesulfonyl chloride (3.34 g, 10.85 mmol, 1.0 equiv, CAS #1070972-67-6). The resulting mixture was stirred for 21 h while being allowed to warm to room temperature. Upon full conversion, as determined by LCMS analysis of the reaction mixture, 1.2M aqueous hydrochloric acid (150 mL) was added. The aqueous phase was extracted with ethyl acetate and the combined organic layers were washed twice with 1.2M aqueous hydrochloride (25 mL each) and once with brine (50 mL). The combined organic layers were dried over magnesium sulfate and concentrated to afford (R)-2-((5-bromo-4-chloro-2-fluorophenyl)sulfonamido)-2-cyclohexylacetamide (4.51 g) which was used directly without purification in the next step. ESI MS m/z=427.1 [M+H]⁺.

Step 4

In a 250 mL round bottom flask equipped with a stir bar and a reflux condenser, (R)-2-((5-bromo-4-chloro-2-fluorophenyl)sulfonamido)-2-cyclohexylacetamide was dissolved in tetrahydrofuran (42.2 mL, 0.25M) under a nitrogen atmosphere. Next, borane-dimethyl sulfide complex (3.60 g, 4.51 mL, 4.5 equiv) was added, and the mixture was heated at 55° C. for 17 h. Upon cooling to room temperature, the reaction was slowly quenched with water (15 mL) and diluted with saturated aqueous sodium bicarbonate (200 mL) and dichloromethane (125 mL) and the layers were separated. The aqueous phase was extracted with dichloromethane and the combined organic layers were dried over magnesium sulfate. Purification by silica gel column chromatography (gradient elution, 0 to 20% MeOH/dichloromethane) afforded (R)-N-(2-amino-1-cyclohexylethyl)-5-bromo-4-chloro-2-fluorobenzenesulfonamide (1.47 g, 34% yield). ESI MS m/z=413.3[M+H]⁺.

Step 5

In a 250 mL round bottom flask equipped with a stir bar and a reflux condenser, (R)-N-(2-amino-1-cyclohexylethyl)-5-bromo-4-chloro-2-fluorobenzenesulfonamide was dissolved in dimethyl sulfoxide (35.5 mL, 0.1M). To the resulting solution was added N,N-diisopropylethylamine (1.38 g, 1.86 mL, 3.0 equiv). The mixture was heated at 55° C. until LCMS analysis indicated full consumption of the starting material and formation of the intermediate (R)-8-bromo-7-chloro-3-cyclohexyl-2,3,4,5-tetrahydrobenzo[f][1,2,5]thiadiazepine 1,1-dioxide.

Step 6

The solution formed above in step 5 was charged with cesium carbonate (4.63 g, 14.21 mmol. 4.0 equiv) prior to the addition of iodomethane (504 mg, 0.22 mL, 3.55 mmol, 1.0 equiv). Upon full consumption of the starting material, as determined by LCMS analysis, the reaction mixture was diluted with water and methyl tert-butyl ether. The layers were separated and the aqueous phase was extracted with methyl tert-butyl ether. The combined organic layers were dried over magnesium sulfate, concentrated, and purified by silica gel column chromatography (cyclohexane/ethyl acetate) to afford (R)-8-bromo-7-chloro-3-cyclohexyl-2-methyl-2,3,4,5-tetrahydrobenzo[f][1,2,5]thiadiazepine 1,1-dioxide (1.07 g, 74% yield). ESI MS m/z=406.9 [M+H]⁺.

Step 7

In a Schlenk tube equipped with a stir bar, (R)-8-bromo-7-chloro-3-cyclohexyl-2-methyl-2,3,4,5-tetrahydrobenzo[f][1,2,5]thiadiazepine 1,1-dioxide (1.05 g, 2.58 mmol, 1.0 equiv), (4-fluoro-3-(methoxycarbonyl)phenyl)boronic acid (701.0 mg, 3.54 mmol, 1.37 equiv, CAS #874219-35-9), cesium carbonate (2.52 g, 7.74 mmol, 3.0 equiv), and bis(triphenylphosphine)palladium(II) chloride (91.0 mg, 5 mol %) were combined neat under a nitrogen atmosphere. Next, 1,4-dioxane (22.4 mL) and water (3.4 mL) were added, and the mixture was heated at 83° C. (external oil bath temperature) for 45 minutes. Upon cooling to room temperature, the mixture was diluted with methyl tert-butyl ether and brine. The aqueous phase was extracted with methyl tert-butyl ether, and the combined organic layers were dried over magnesium sulfate. Concentration afforded a crude residue which was purified by silica gel column chromatography (cyclohexane/ethyl acetate) to afford methyl (R)-5-(7-chloro-3-cyclohexyl-2-methyl-1,1-dioxido-2,3,4,5-tetrahydrobenzo[f][1,2,5]thiadiazepin-8-yl)-2-fluorobenzoate (1.19 g, 96% yield). ESI MS m/z=481.1 [M+H]⁺.

Step 8

In a 4 mL vial equipped with a stir bar, (R)-5-(7-chloro-3-cyclohexyl-2-methyl-1,1-dioxido-2,3,4,5-tetrahydrobenzo[f][1,2,5]thiadiazepin-8-yl)-2-fluorobenzoate (20.0 mg, 0.042 mmol, 1.0 equiv), cesium carbonate (67.7 mg, 0.21 mmol, 5.0 equiv), and Rac-BINAP-Pd-G4 (6.28 mg, 15 mol %, CAS#1599466-90-6) were combined neat under a nitrogen atmosphere. Next, 3-bromo-5-fluoropyridine (22.0 mg, 0.125 mmol, 3.0 equiv) was added as a solution in toluene (0.83 mL). The vial was then sealed with electrical tape and heated at 115° C. for 15 h. Upon cooling to room temperature, the reaction mixture was concentrated, and the residue was re-dissolved in N,N-dimethylformamide and passed through a 0.45 micron syringe filter and purified by RPHPLC to afford methyl (R)-5-(7-chloro-3-cyclohexyl-5-(5-fluoropyridin-3-yl)-2-methyl-1,1-dioxido-2,3,4,5-tetrahydrobenzo[f][1,2,5]thiadiazepin-8-yl)-2-fluorobenzoate.

Step 9

The (R)-5-(7-chloro-3-cyclohexyl-5-(5-fluoropyridin-3-yl)-2-methyl-1,1-dioxido-2,3,4,5-tetrahydrobenzo[f][1,2,5]thiadiazepin-8-yl)-2-fluorobenzoate formed above in step 8 was dissolved in 1,4-dioxane (1.0 mL) and water (0.5 mL). Lithium hydroxide (10.0 mg, 10.0 equiv) was added, and the resulting mixture was stirred for 16 h at room temperature. The reaction mixture was quenched with formic acid (0.25 mL) and purified by RPHPLC. The product was lyophilized from acetonitrile and water to afford (R)-5-(7-chloro-3-cyclohexyl-5-(5-fluoropyridin-3-yl)-2-methyl-1,1-dioxido-2,3,4,5-tetrahydrobenzo[f][1,2,5]thiadiazepin-8-yl)-2-fluorobenzoic acid, Ex.280 (12.3 mg, 53% over two steps). ESI MS m/z=562.2 [M+H]⁺.

Examples 281-303 were prepared using procedures analogous to that used for Ex.280.

| Example | Structure | MS |
|---|---|---|
| 280 | 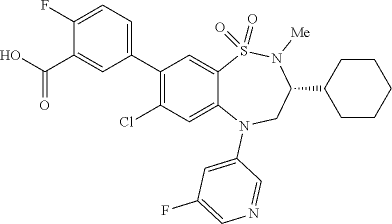 [SMILES: CN1[C@H](C2CCCCC2)CN(c2cncc(F)c2)c2cc(Cl)c(-c3ccc(F)c(C(=O)O)c3)cc2S1(=O)=O] | [M + H]⁺ 562.2 |
| 281 | 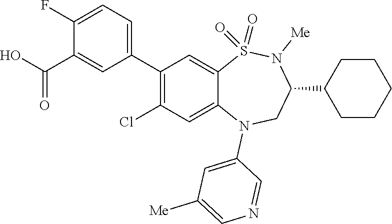 [SMILES: Cc1cncc(N2C[C@@H](C3CCCCC3)N(C)S(=O)(=O)c3cc(-c4ccc(F)c(C(=O)O)c4)c(Cl)cc32)c1] | [M + H]⁺ 558.4 |
| 282 | 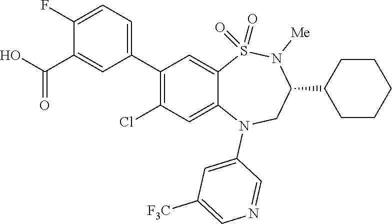 [SMILES: CN1[C@H](C2CCCCC2)CN(c2cncc(C(F)(F)F)c2)c2cc(Cl)c(-c3ccc(F)c(C(=O)O)c3)cc2S1(=O)=O] | [M + H]⁺ 612.2 |
| 283 | 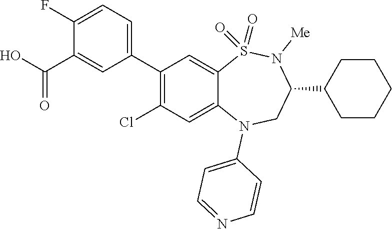 [SMILES: CN1[C@H](C2CCCCC2)CN(c2ccncc2)c2cc(Cl)c(-c3ccc(F)c(C(=O)O)c3)cc2S1(=O)=O] | [M + H]⁺ 544.2 |
| 284 | 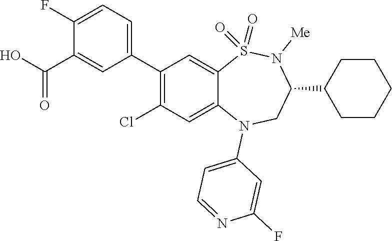 [SMILES: CN1[C@H](C2CCCCC2)CN(c2ccnc(F)c2)c2cc(Cl)c(-c3ccc(F)c(C(=O)O)c3)cc2S1(=O)=O] | [M + H]⁺ 562.3 |
| 285 | 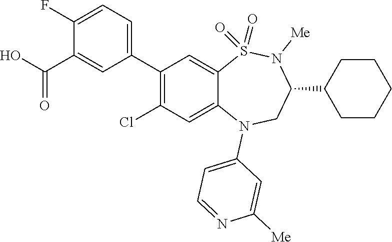 [SMILES: Cc1cc(N2C[C@@H](C3CCCCC3)N(C)S(=O)(=O)c3cc(-c4ccc(F)c(C(=O)O)c4)c(Cl)cc32)ccn1] | [M + H]⁺ 558.1 |
| 286 | 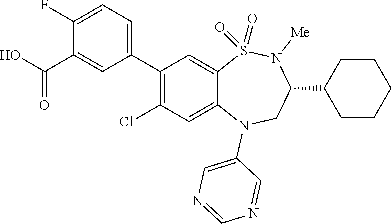 [SMILES: CN1[C@H](C2CCCCC2)CN(c2cncnc2)c2cc(Cl)c(-c3ccc(F)c(C(=O)O)c3)cc2S1(=O)=O] | [M + H]⁺ 545.2 |
| 287 | 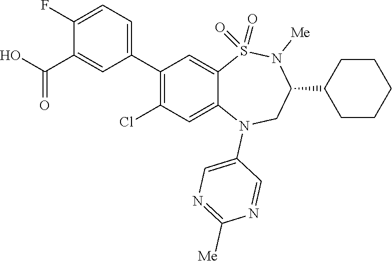 [SMILES: Cc1ncc(N2C[C@@H](C3CCCCC3)N(C)S(=O)(=O)c3cc(-c4ccc(F)c(C(=O)O)c4)c(Cl)cc32)cn1] | [M + H]⁺ 559.2 |
| 288 | 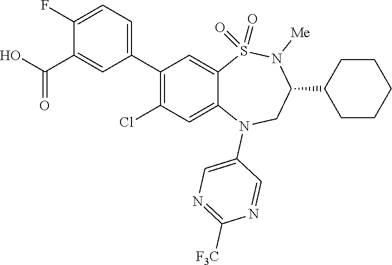 [SMILES: CN1[C@H](C2CCCCC2)CN(c2cnc(C(F)(F)F)nc2)c2cc(Cl)c(-c3ccc(F)c(C(=O)O)c3)cc2S1(=O)=O] | [M + H]⁺ 613.3 |
| 289 | 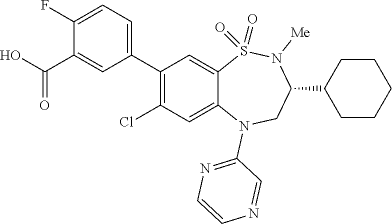 [SMILES: CN1[C@H](C2CCCCC2)CN(c2cnccn2)c2cc(Cl)c(-c3ccc(F)c(C(=O)O)c3)cc2S1(=O)=O] | [M + H]⁺ 545.4 |
| 290 | 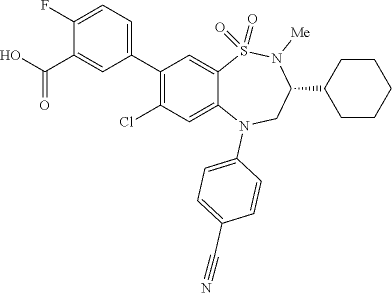 [SMILES: CN1[C@H](C2CCCCC2)CN(c2ccc(C#N)cc2)c2cc(Cl)c(-c3ccc(F)c(C(=O)O)c3)cc2S1(=O)=O] | [M + H]⁺ 568.2 |
| 291 | 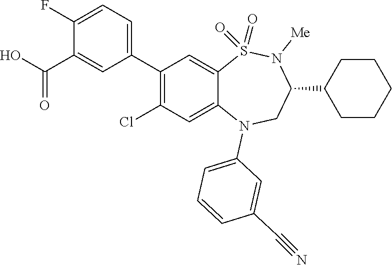 [SMILES: CN1[C@H](C2CCCCC2)CN(c2cccc(C#N)c2)c2cc(Cl)c(-c3ccc(F)c(C(=O)O)c3)cc2S1(=O)=O] | [M + H]⁺ 568.0 |
| 292 | 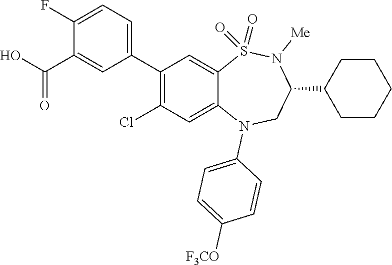 [SMILES: CN1[C@H](C2CCCCC2)CN(c2ccc(OC(F)(F)F)cc2)c2cc(Cl)c(-c3ccc(F)c(C(=O)O)c3)cc2S1(=O)=O] | [M + H]⁺ 626.9 |
| 293 | 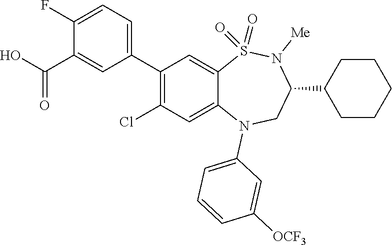 [SMILES: CN1[C@H](C2CCCCC2)CN(c2cccc(OC(F)(F)F)c2)c2cc(Cl)c(-c3ccc(F)c(C(=O)O)c3)cc2S1(=O)=O] | [M + H]⁺ 627.4 |
| 294 | 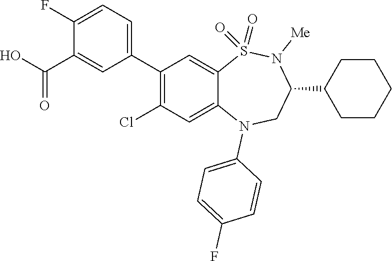 [SMILES: CN1[C@H](C2CCCCC2)CN(c2ccc(F)cc2)c2cc(Cl)c(-c3ccc(F)c(C(=O)O)c3)cc2S1(=O)=O] | [M + H]⁺ 561.2 |
| 295 | 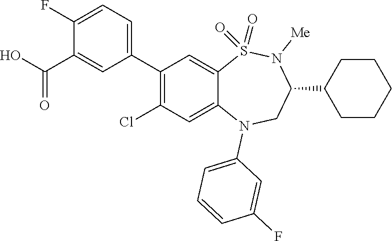 [SMILES: CN1[C@H](C2CCCCC2)CN(c2cccc(F)c2)c2cc(Cl)c(-c3ccc(F)c(C(=O)O)c3)cc2S1(=O)=O] | [M + H]⁺ 561.2 |
| 296 | 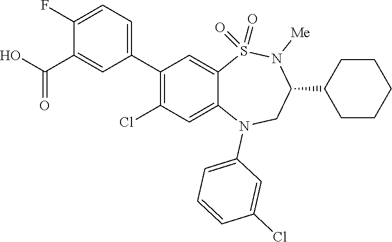 [SMILES: CN1[C@H](C2CCCCC2)CN(c2cccc(Cl)c2)c2cc(Cl)c(-c3ccc(F)c(C(=O)O)c3)cc2S1(=O)=O] | [M + H]⁺ 577.1 |
| 297 | 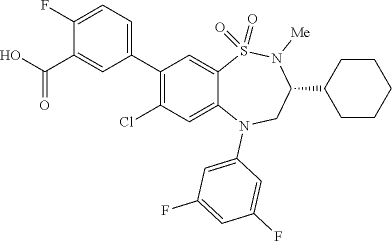 [SMILES: CN1[C@H](C2CCCCC2)CN(c2cc(F)cc(F)c2)c2cc(Cl)c(-c3ccc(F)c(C(=O)O)c3)cc2S1(=O)=O] | [M + H]⁺ 579.2 |
| 298 | 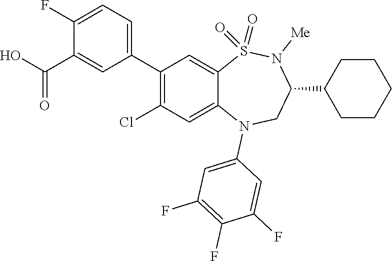 [SMILES: CN1[C@H](C2CCCCC2)CN(c2cc(F)c(F)c(F)c2)c2cc(Cl)c(-c3ccc(F)c(C(=O)O)c3)cc2S1(=O)=O] | [M + H]⁺ 597.2 |
| 299 | 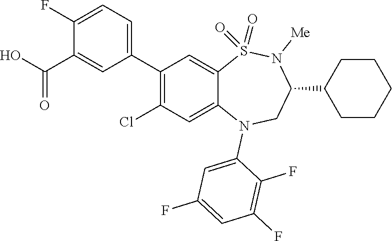 [SMILES: CN1[C@H](C2CCCCC2)CN(c2cc(F)cc(F)c2F)c2cc(Cl)c(-c3ccc(F)c(C(=O)O)c3)cc2S1(=O)=O] | [M + H]⁺ 597.2 |
| 300 | 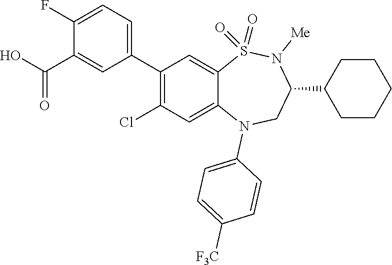 [SMILES: CN1[C@H](C2CCCCC2)CN(c2ccc(C(F)(F)F)cc2)c2cc(Cl)c(-c3ccc(F)c(C(=O)O)c3)cc2S1(=O)=O] | [M + H]⁺ 611.2 |
| 301 | 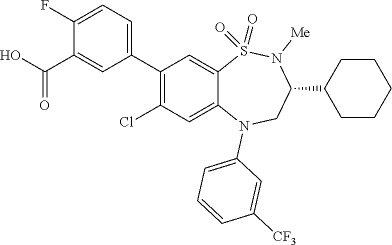 [SMILES: CN1[C@H](C2CCCCC2)CN(c2cccc(C(F)(F)F)c2)c2cc(Cl)c(-c3ccc(F)c(C(=O)O)c3)cc2S1(=O)=O] | [M + H]⁺ 611.2 |
| 302 | 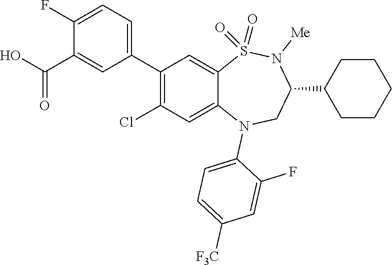 [SMILES: CN1[C@H](C2CCCCC2)CN(c2ccc(C(F)(F)F)cc2F)c2cc(Cl)c(-c3ccc(F)c(C(=O)O)c3)cc2S1(=O)=O] | [M + H]⁺ 629.2 |
| 303 | 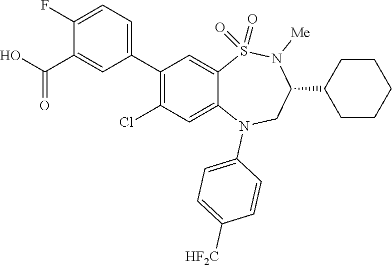 | [M + H]⁺ 593.2 |

### Ex.304: Synthesis of(R)-5-(7-chloro-3-cyclohexyl-5-(2-methoxyphenyl)-2-methyl-1,1-dioxido-2,3,4,5-tetrahydrobenzo[f][1,2,5]thiadiazepin-8-yl)-2-fluorobenzoic acid

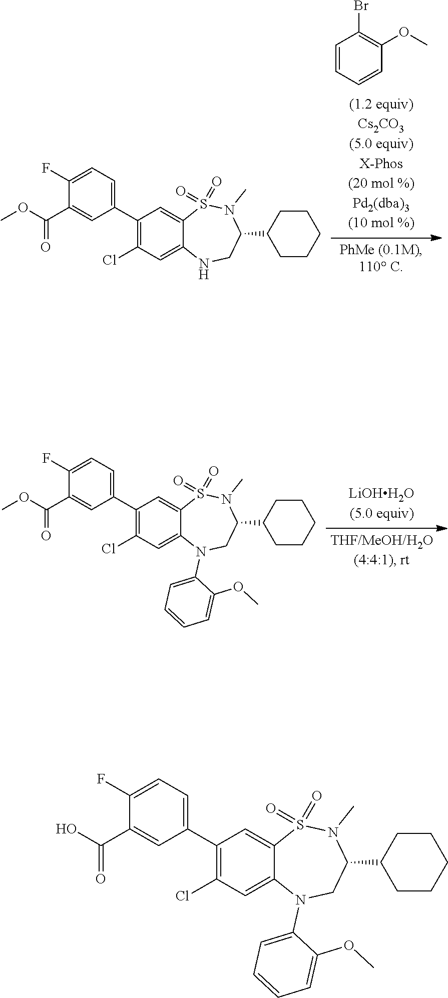

Step 1

To a solution of methyl (R)-5-(7-chloro-3-cyclohexyl-2-methyl-1,1-dioxido-2,3,4,5-tetrahydrobenzo[f][1,2,5]thiadiazepin-8-yl)-2-fluorobenzoate (200.0 mg, 0.4 mmol, 1.0 eq) in toluene (4.0 mL) was added 1-bromo-2-methoxybenzene (233.4 mg, 1.2 mmol, 3.0 eq), Cs₂CO₃ (677.7 mg, 2.0 mmol, 5.0 eq), Pd₂(dba)₃ (38.4 mg, 0.04 mmol, 0.1 eq) and X-Phos (39.5 mg, 0.08 mmol, 0.2 eq) under N₂ atmosphere. The reaction mixture was then stirred at 110° C. overnight. The mixture was diluted with water (10 mL) and extracted three times with EtOAc (20 mL each). The combined organic phase was washed with brine (10 mL), dried over Na₂SO₄ and concentrated to give a residue which was purified by silica gel column chromatography (petroleum ether: EtOAc=20:1) to give methyl (R)-5-(7-chloro-3-cyclohexyl-5-(2-methoxyphenyl)-2-methyl-1,1-dioxido-2,3,4,5-tetrahydrobenzo[f][1,2,5]thiadiazepin-8-yl)-2-fluorobenzoate (150 mg, 61% yield) as a white solid. ESI MS m/z=587.1 [M+H]⁺. ¹H NMR (400 MHz, DMSO-d₆): δ 7.85 (dd, J=6.8, 2.4 Hz, 1H), 7.80-7.68 (m, 1H), 7.62 (s, 1H), 7.53-7.31 (m, 3H), 7.31-7.18 (m, 1H), 7.09 (t, J=7.6 Hz, 1H), 6.26 (s, 1H), 4.64-4.42 (m, 1H), 3.87 (s, 3H), 3.81 (s, 3H), 3.33 (s, 1H), 3.28-3.13 (m, 1H), 3.02 (s, 3H), 2.12-1.92 (m, 1H), 1.83-1.47 (m, 5H), 1.26-0.99 (m, 4H), 0.87-0.72 (m, 1H).

Step 2

To a solution of methyl (R)-5-(7-chloro-3-cyclohexyl-5-(2-methoxyphenyl)-2-methyl-1,1-dioxido-2,3,4,5-tetrahydrobenzo[f][1,2,5]thiadiazepin-8-yl)-2-fluorobenzoate (150.0 mg, 0.25 mmol, 1.0 eq) in THF/MeOH/H₂O (2/2/0.5 mL) was added LiOH H₂O (53.5 mg, 1.25 mmol, 5.0 eq). Then the reaction mixture was stirred overnight at room temperature. The reaction mixture was concentrated under reduced pressure and the mixture was adjusted to pH 4 with HCl (1 N). The resulting precipitate was collected via filtration and dried by lyophilization to give (R)-5-(7-chloro-3-cyclohexyl-5-(2-methoxyphenyl)-2-methyl-1,1-dioxido-2,3,4,5-tetrahydrobenzo[f][1,2,5]thiadiazepin-8-yl)-2-fluorobenzoic acid, Ex.304 (66 mg, 45% yield) as a yellow solid. ESI MS m/z=573.1 [M+H]⁺. ¹H NMR (300 MHz, DMSO-d₆): δ 13.34 (s, 1H), 7.85-7.81 (m, 1H), 7.72-7.65 (m, 1H), 7.61 (s, 1H), 7.43-7.34 (m, 3H), 7.24 (d, J=8.1 Hz, 1H), 7.09 (t, J=7.5 Hz, 1H), 6.25 (s, 1H), 4.52 (s, 1H), 3.81 (s, 3H), 3.32-3.28 (m, 1H), 3.21 (s, 1H), 3.02 (s, 3H), 2.05-1.97 (m, 1H), 1.78-1.54 (m, 5H), 1.27-0.99 (m, 5H).

Example 305 was prepared using a procedure analogous to that used for Ex.304.

| Example | Structure | MS | NMR |
|---|---|---|---|
| 304 | 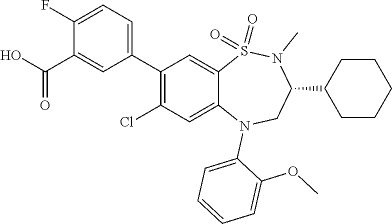 [SMILES: COc1ccccc1N1C[C@@H](C2CCCCC2)N(C)S(=O)(=O)c2cc(-c3ccc(F)c(C(=O)O)c3)c(Cl)cc21] | [M + H]⁺ 573.1 | ¹H NMR (300 MHz, DMSO-d₆): δ 13.34 (s, 1H), 7.85 − 7.81 (m, 1H), 7.72 − 7.65 (m, 1H), 7.61 (s, 1H), 7.43 − 7.34 (m, 3H), 7.24 (d, J = 8.1 Hz, 1H), 7.09 (t, J = 7.5 Hz, 1H), 6.25 (s, 1H), 4.52 (s, 1H), 3.81 (s, 3H), 3.32 − 3.28 (m, 1H), 3.21 (s, 1H), 3.02 (s, 3H), 2.05 − 1.97 (m, 1H), 1.78 − 1.54 (m, 5H), 1.27 − 0.99 (m, 5H) |
| 305 | 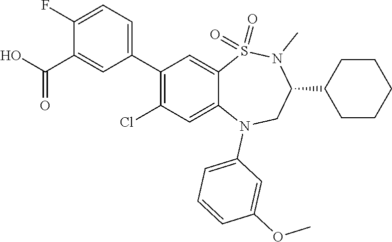 [SMILES: COc1cccc(N2C[C@@H](C3CCCCC3)N(C)S(=O)(=O)c3cc(-c4ccc(F)c(C(=O)O)c4)c(Cl)cc32)c1] | [M + H]⁺ 573.1 | ¹H NMR (300 MHz, DMSO-d₆): δ 13.47 (s, 1H), 7.97 − 7.87 (m, 1H), 7.83 − 7.73 (m, 2H), 7.50 − 7.32 (m, 2H), 7.20 (t, J = 8.1 Hz, 1H), 6.61 − 6.40 (m, 3H), 4.38 − 4.32 (m, 1H), 3.73 (s, 3H), 3.63 − 3.40 (m, 1H), 3.28 − 3.11 (m, 1H), 2.66 (s, 3H), 2.03 − 1.83 (m, 2H), 1.77 − 1.53 (m, 4H), 1.26 − 1.14 (m, 3H), 1.06 − 0.86 (m, 2H) |

### Ex.306: Synthesis of (R)-5-(7-chloro-3-cyclohexyl-5-(4-methoxyphenyl)-2-methyl-1,1-dioxido-2,3,4,5-tetrahydrobeuzo[f][1,2,5]thiadiazepin-8-yl)-2-fluorobenzoic acid

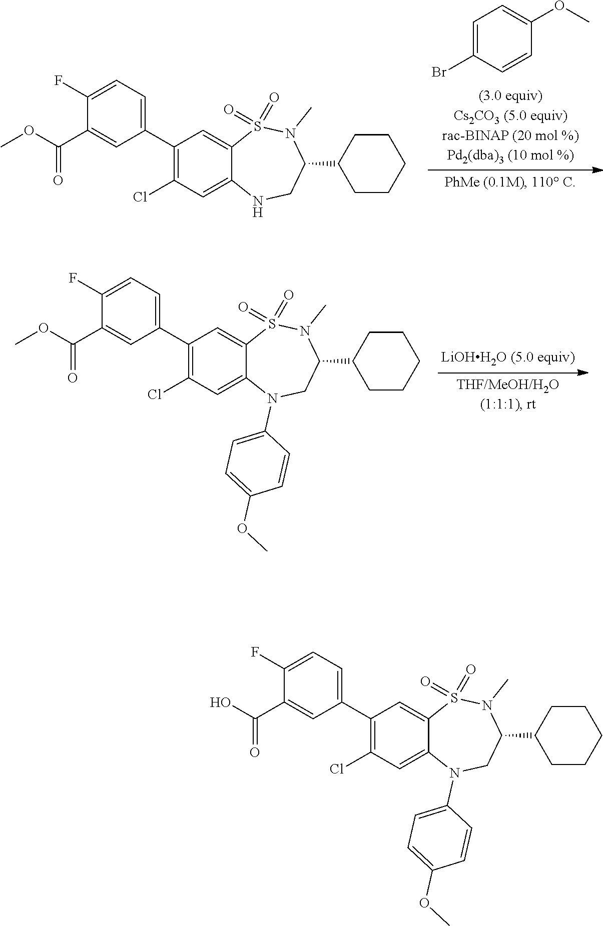

Step 1

A mixture of methyl (R)-5-(7-chloro-3-cyclohexyl-2-methyl-1,1-dioxido-2,3,4,5-tetrahydrobenzo[f][1,2,5]thiadiazepin-8-yl)-2-fluorobenzoate (80.0 mg, 0.17 mmol, 1.0 eq), 1-bromo-4-methoxybenzene (93.1 mg, 0.50 mmol, 3.0 eq), Cs₂CO₃ (270.0 mg, 0.83 mmol, 5.0 eq), Pd₂(dba)₃ (15.6 mg, 0.017 mmol, 0.1 eq) and rac-BINAP (20.5 mg, 0.033 mmol, 0.2 eq) in toluene (5.0 mL) was heated at 110° C. overnight under nitrogen atmosphere. The resulting mixture was cooled to room temperature and poured into water (30 mL). The aqueous phase was extracted twice with EtOAc (50 mL each), and the combined organic layers were dried over Na₂SO₄ and concentrated. The residue was purified by silica gel column chromatography (petroleum ether: EtOAc=3:1) to give afford methyl (R)-5-(7-chloro-3-cyclohexyl-5-(4-methoxyphenyl)-2-methyl-1,1-dioxido-2,3,4,5-tetrahydrobenzo[f][1,2,5]thiadiazepin-8-yl)-2-fluorobenzoate (40.0 mg, 41% yield) as a yellow solid. ¹H NMR (400 MHz, CDCl₃): δ 7.99 (dd, J=6.8, 2.4 Hz, 1H), 7.80 (s, 1H), 7.62-7.54 (m, 1H), 7.21-7.14 (m, 1H), 7.14-7.06 (m, 2H), 6.98-6.90 (m, 2H), 6.78 (s, 1H), 4.50-4.29 (m, 1H), 4.18-4.02 (m, 1H), 3.95 (s, 3H), 3.84 (s, 3H), 3.14 (t, J=10.4 Hz, 1H), 2.99 (s, 3H), 2.27-2.14 (m, 1H), 1.93-1.60 (m, 5H), 1.33-1.05 (m, 3H), 1.00-0.77 (m, 2H).

Step 2

A mixture of methyl (R)-5-(7-chloro-3-cyclohexyl-5-(4-methoxyphenyl)-2-methyl-1,1-dioxido-2,3,4,5-tetrahydrobenzo[f][1,2,5]thiadiazepin-8-yl)-2-fluorobenzoate (40.0 mg, 0.07 mmol, 0.1 eq) and LiOH—H₂O (14.7 mg, 0.35 mmol, 5.0 eq) in THE (1.0 mL), MeOH (1.0 mL) and H₂O (1.0 mL) was stirred for 3 h at room temperature. The resulting mixture was concentrated under vacuum and diluted with H₂O (5 mL). The mixture was adjusted to pH 2-3 with 1 N HCl. The resulting precipitate was collected via filtration to afford (R)-5-(7-chloro-3-cyclohexyl-5-(4-methoxyphenyl)-2-methyl-1,1-dioxido-2,3,4,5-tetrahydrobenzo[f][1,2,5]thiadiazepin-8-yl)-2-fluorobenzoic acid, Ex.306 (17.5 mg, 45% yield) as a white solid. ESI MS m/z=573.2 [M+H]⁺. ¹H NMR (300 MHz, DMSO-d₆): δ 13.38 (s, 1H), 7.87 (dd, J=6.9, 2.4 Hz, 1H), 7.79-7.62 (m, 2H), 7.48-7.35 (m, 1H), 7.30-7.14 (m, 2H), 7.06-6.94 (m, 2H), 6.80 (s, 1H), 4.27-3.98 (m, 2H), 3.77 (s, 3H), 3.28-3.13 (m, 1H), 2.87 (s, 3H), 2.04-1.88 (m, 1H), 1.82-1.50 (m, 5H), 1.30-0.77 (m, 5H).

| Example | Structure | MS | NMR |
|---|---|---|---|
| 306 | 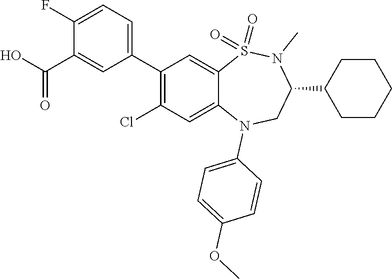 [SMILES: COc1ccc(N2C[C@@H](C3CCCCC3)N(C)S(=O)(=O)c3cc(-c4ccc(F)c(C(=O)O)c4)c(Cl)cc32)cc1] | [M + H]⁺ 573.2 | ¹H NMR (300 MHz, DMSO-d₆): δ Hz, 1H), 7.79 − 7.62 (m, 2H), 7.48 − 7.35 (m, 1H), 7.30 − 7.14 (m, 2H), 7.06 − 6.94 (m, 2H), 6.80 (s, 1H), 4.27 − 3.98 (m, 2H), 3.77 (s, 3H), 3.28 − 3.13 (m, 1H), 2.87 (s, 3H), 2.04 − 1.88 (m, 1H), 1.82 − 1.50 (m, 5H), 1.30 − 0.77 (m, 5H) |

The following compounds were prepared using a procedure analogous to that used for Ex.306.

| Example | Structure | MS | NMR |
|---|---|---|---|
| 307 | 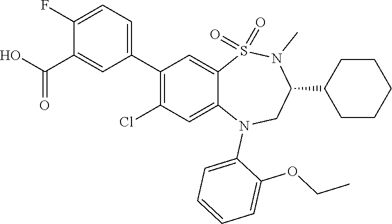 [SMILES: CCOc1ccccc1N1C[C@@H](C2CCCCC2)N(C)S(=O)(=O)c2cc(-c3ccc(F)c(C(=O)O)c3)c(Cl)cc21] | [M + H]⁺ 587.0 | ¹H NMR (300 MHz, DMSO-d₆): δ 13.43 (s, 1H), 7.85-7.78 (m, 1H), 7.69-7.60 (m, 2H), 7.46-7.26 (m, 3H), 7.24-7.13 (m, 1H), 7.07 (t, J = 7.5 Hz, 1H), 6.33 (s, 1H), 4.58-4.41 (m, 1H), 4.22-3.82 (m, 2H), 3.24-3.11 (m, 1H), 3.01 (s, 3H), 2.03-1.99 (m, 1H), 1.77- 1.53 (m, 5H), 1.26-0.95 (m, 7H), 0.88-0.72 (m, 1H) |
| 308 | 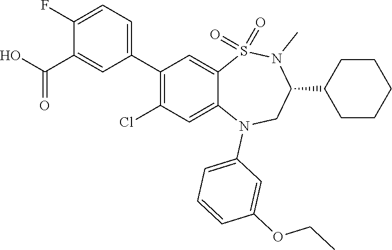 [SMILES: CCOc1cccc(N2C[C@@H](C3CCCCC3)N(C)S(=O)(=O)c3cc(-c4ccc(F)c(C(=O)O)c4)c(Cl)cc32)c1] | [M − H]⁻ 585.0 | ¹H NMR (300 MHz, DMSO-d₆): δ 7.94 (d, J = 5.4 Hz, 1H), 7.84- 7.69 (m, 2H), 7.58-7.43 (m, 1H), 7.32 (s, 1H), 7.20 (t, J = 8.1 Hz, 1H), 6.64-6.35 (m, 3H), 4.38- 4.32 (m, 1H), 4.07-3.91 (m, 2H), 3.67-3.54 (m, 1H), 3.47-3.38 (m, 1H), 2.68 (s, 3H), 2.02-1.82 (m, 2H), 1.78-1.67 (m, 2H), 1.65- 1.52 (m, 2H), 1.30 (t, J = 6.9 Hz, 3H), 1.24-1.08 (m, 3H), 1.08- 0.88 (m, 2H). |
| 309 | 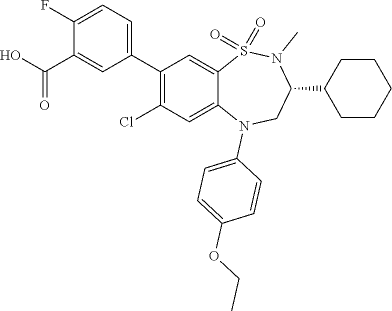 [SMILES: CCOc1ccc(N2C[C@@H](C3CCCCC3)N(C)S(=O)(=O)c3cc(-c4ccc(F)c(C(=O)O)c4)c(Cl)cc32)cc1] | [M + H]⁺ 587.2 | ¹H NMR (400 MHz, DMSO-d₆): δ 13.42 (s, 1H), 7.93- 7.80 (m, 1H), 7.75-7.61 (m, 2H), 7.49-7.31 (m, 1H), 7.20 (d, J = 8.0 Hz, 2H), 6.97 (d, J = 8.8 Hz, 2H), 6.78 (s, 1H), 4.13 (s, 2H), 4.03 (q, J = 6.8 Hz, 2H), 3.23 (s, 1H), 2.86 (s, 3H), 2.05-1.89 (m, 1H), 1.81-1.53 (m, 5H), 1.33 (t, J = 6.8 Hz, 3H), 1.20-1.06 (m, 3H), 1.06- 0.94 (m, 1H), 0.90-0.78 (m, 1H) |
| 310 | 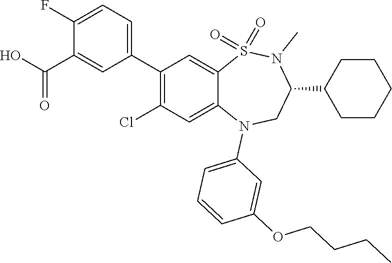 [SMILES: CCCCOc1cccc(N2C[C@@H](C3CCCCC3)N(C)S(=O)(=O)c3cc(-c4ccc(F)c(C(=O)O)c4)c(Cl)cc32)c1] | [M − H]⁻ 613.1 | ¹H NMR (400 MHz, DMSO-d₆): δ 7.87 (d, J = 6.4 Hz, 1H), 7.77- 7.63 (m, 2H), 7.41-7.26 (m, 2H), 7.19 (t, J = 8.0 Hz, 1H), 6.64-6.36 (m, 3H), 4.46-4.28 (m, 1H), 3.99- 3.82 (m, 2H), 3.80-3.40 (m, 1H), 2.67 (s, 3H), 2.02-1.84 (m, 2H), 1.80-1.48 (m, 6H), 1.45- 1.35 (m, 2H), 1.31-1.08 (m, 4H), 1.02-0.96 (m, 1H), 0.92 (t, J = 7.2 Hz, 3H). |
| 311 | 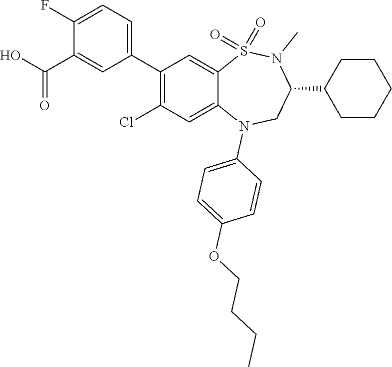 [SMILES: CCCCOc1ccc(N2C[C@@H](C3CCCCC3)N(C)S(=O)(=O)c3cc(-c4ccc(F)c(C(=O)O)c4)c(Cl)cc32)cc1] | [M + H]⁺ 615.3 | ¹H NMR (300 MHz, DMSO-d6): δ 13.44 (s, 1H), 7.87-7.85 (m, 1H), 7.77-7.58 (m, 2H), 7.48-7.30 (m, 1H), 7.19 (d, J = 8.4 Hz, 2H), 6.97 (d, J = 8.7 Hz, 2H), 6.78 (s, 1H), 4.13 (s, 2H), 3.97 (t, J = 6.3 Hz, 2H), 3.29-3.11 (m, 1H), 2.86 (s, 3H), 2.08-1.85 (m, 1H), 1.82- 1.53 (m, 7H), 1.53-1.34 (m, 2H), 1.29-1.04 (m, 4H), 0.95 (t, J = 7.8 Hz, 3H), 0.88-0.78 (m, 1H). |
| 312 | 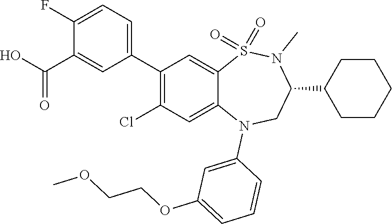 [SMILES: COCCOc1cccc(N2C[C@@H](C3CCCCC3)N(C)S(=O)(=O)c3cc(-c4ccc(F)c(C(=O)O)c4)c(Cl)cc32)c1] | [M + H]⁺ 617.1 | ¹H NMR (300 MHz, DMSO-d₆): δ 7.90-7.84 (m, 1H), 7.76-7.65 (m, 2H), 7.42-7.30 (m, 2H), 7.20 (t, J = 8.1 Hz, 1H), 6.60-6.40 (m, 3H), 4.48-4.29 (m, 1H), 4.10- 4.01 (m, 2H), 3.64-3.33 (m, 3H), 3.30-3.26 (m, 4H), 2.67 (s, 3H), 2.03-1.84 (m, 2H), 1.75-1.56 (m, 4H), 1.23-1.14 (m, 3H), 1.04- 0.91 (m, 2H). |
| 313 | 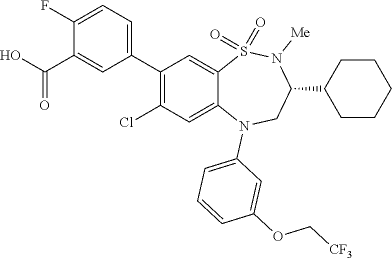 [SMILES: CN1[C@H](C2CCCCC2)CN(c2cccc(OCC(F)(F)F)c2)c2cc(Cl)c(-c3ccc(F)c(C(=O)O)c3)cc2S1(=O)=O] | [M + H]⁺ 641.0 | ¹H NMR (400 MHz, DMSO-d6): δ 13.46 (s, 1H), 7.98-7.91 (m, 1H), 7.83-7.74 (m, 2H), 7.49-7.33 (m, 2H), 7.30-7.19 (m, 1H), 6.69- 6.55 (m, 3H), 4.78-4.65 (m, 2H), 4.37-4.23 (m, 1H), 3.51 (s, 1H), 3.36 (s, 1H), 2.66 (s, 3H), 2.06-1.85 (m, 2H), 1.73-1.55 (m, 4H), 1.23-0.91 (m, 5H). |
| 314 | 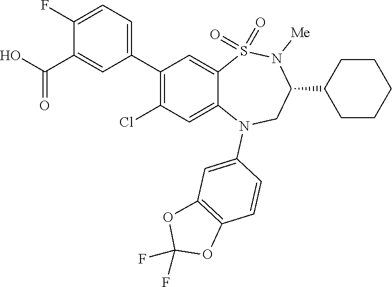 [SMILES: CN1[C@H](C2CCCCC2)CN(c2ccc3c(c2)OC(F)(F)O3)c2cc(Cl)c(-c3ccc(F)c(C(=O)O)c3)cc2S1(=O)=O] | [M + H]⁺ 623.0 | ¹H NMR (400 MHz, DMSO-d₆): δ 7.74 (s, 1H), 7.69 (s, 1H), 7.44 (s, 1H), 7.31 (d, J = 8.8 Hz, 2H), 7.23- 7.10 (m, 2H), 6.74 (s, 1H), 4.34- 4.28 (m, 1H), 3.67 (s, 1H), 3.31- 3.22 (m, 1H), 2.68 (s, 3H), 2.02- 1.86 (m, 2H), 1.73-1.55 (m, 4H), 1.24-0.95 (m, 5H). |
| 315 | 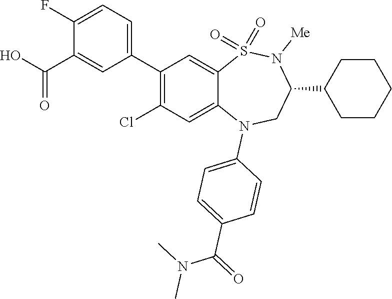 [SMILES: CN(C)C(=O)c1ccc(N2C[C@@H](C3CCCCC3)N(C)S(=O)(=O)c3cc(-c4ccc(F)c(C(=O)O)c4)c(Cl)cc32)cc1] | [M + H]⁺ 614.2 | ¹H NMR (300 MHz, DMSO-d₆): δ 7.76 (s, 1H), 7.71 (s, 1H), 7.57 (s, 1H), 7.49-7.41 (m, 1H), 7.30- 7.36 (m, 2H), 7.24-7.14 (m, 1H), 6.76-6.65 (m, 2H), 4.43-4.27 (m, 1H), 3.52-3.41 (m, 2H), 2.96 (s, 6H), 2.58 (s, 3H), 2.13-2.03 (m, 1H), 1.87-1.53 (m, 5H), 1.30- 0.99 (m, 5H). |

### Ex.316: Synthesis of (R)-5-(7-chloro-5-(5-chlorothiophen-3-yl)-3-cyclohexyl-2-methyl-1,1-dioxido-2,3,4,5-tetrahydrobeuzo[f][1,2,5]thiadiazepin-8-yl)-2-fluorobenzoic acid

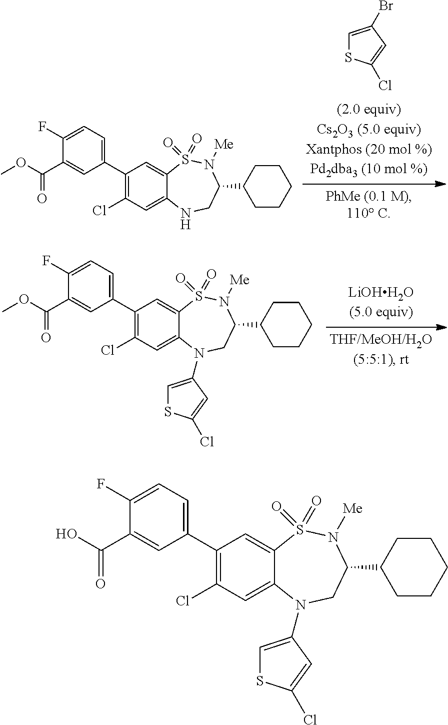

### Step 1

To a solution of methyl (R)-5-(7-chloro-3-cyclohexyl-2-methyl-1,1-dioxido-2,3,4,5-tetrahydrobenzo[f][1,2,5]thiadiazepin-8-yl)-2-fluorobenzoate (200.0 mg, 0.42 mmol, 1.0 eq) in toluene (4 mL) was added 4-bromo-2-chlorothiophene (164.3 mg, 0.83 mmol, 2.0 eq), Cs₂CO₃ (677.7 mg, 2.1 mmol, 5.0 eq), Pd₂(dba)₃ (41 mg, 0.042 mmol, 0.1 eq) and Xantphos (42 mg, 0.084 mmol, 0.2 eq) under N₂ atmosphere. The reaction mixture was then stirred at 110° C. overnight. The mixture was concentrated, then added with water (10 mL) and extracted with EtOAc (20 mL×3). The combined organic phase was washed with brine (10 mL), dried over Na₂SO₄ and concentrated to give a residue. The residue was purified by silica gel column chromatography (Petroleum ether: EtOAc=20:1) to give methyl (R)-5-(7-chloro-5-(5-chlorothiophen-3-yl)-3-cyclohexyl-2-methyl-1,1-dioxido-2,3,4,5-tetrahydrobenzo[f][1,2,5]thiadiazepin-8-yl)-2-fluorobenzoate (30 mg, 12% yield) as a yellow solid. ESI MS m/z=596.4 [M+H]⁺. ¹H NMR (300 MHz, CDCl₃): δ 8.04-7.98 (m, 1H), 7.86 (s, 1H), 7.64-7.57 (m, 1H), 7.23-7.15 (m, 2H), 6.67 (s, 1H), 6.34 (s, 1H), 4.12-4.02 (m, 1H), 3.96 (s, 4H), 3.41-3.25 (m, 1H), 2.82 (s, 3H), 2.19-2.10 (m, 1H), 1.81-1.68 (m, 5H), 1.03-0.92 (m, 3H), 0.87-0.83 (m, 2H).

Step 2

To a solution of methyl (R)-5-(7-chloro-5-(5-chlorothiophen-3-yl)-3-cyclohexyl-2-methyl-1,1-dioxido-2,3,4,5-tetrahydrobenzo[f][1,2,5]thiadiazepin-8-yl)-2-fluorobenzoate (30.0 mg, 0.05 mmol, 1.0 eq) in THE (1 mL), MeOH (1 mL) and H₂O (0.2 mL) was added LiOH·H₂O (10.5 mg, 0.25 mmol, 5.0 eq).

Then the reaction mixture was stirred overnight at room temperature. The reaction mixture was concentrated under reduced pressure, the aqueous was adjusted pH to 4 with HCl (1 N). The mixture was filtered and the filter cake was dried by lyophilization to give (R)-5-(7-chloro-5-(5-chlorothiophen-3-yl)-3-cyclohexyl-2-methyl-1,1-dioxido-2,3,4,5-tetrahydrobenzo[f][1,2,5]thiadiazepin-8-yl)-2-fluorobenzoic acid, Ex.316 (2 mg, 7% yield) as a white solid. ESI MS m/z=582.9 [M+H]⁺. ¹H NMR (300 MHz, DMSO-d₆): δ 7.93-7.87 (m, 1H), 7.75-7.68 (m, 2H), 7.46-7.37 (m, 2H), 6.93 (s, 1H), 6.69 (s, 1H), 4.22-4.10 (m, 1H), 3.78-3.50 (m, 2H), 2.68 (s, 3H), 1.96-1.86 (m, 2H), 1.76-1.56 (m, 4H), 1.23-1.10 (m, 3H), 1.04-0.91 (m, 2H).

Examples 317 and 318 were prepared using procedures analogous to that used for Ex.316.

| Example | Structure | MS | NMR |
|---|---|---|---|
| 316 | 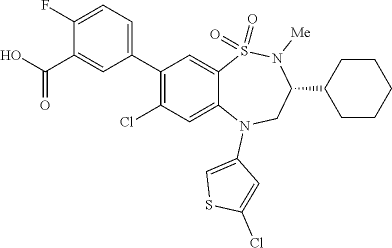 [SMILES: CN1[C@H](C2CCCCC2)CN(c2csc(Cl)c2)c2cc(Cl)c(-c3ccc(F)c(C(=O)O)c3)cc2S1(=O)=O] | [M + H]⁺ 582.9 | ¹H NMR (300 MHz, DMSO- d₆): δ 7.93 − 7.87 (m, 1H), 7.75 − 7.68 (m, 2H), 7.46 − 7.37 (m, 2H), 6.93 (s, 1H), 6.69 (s, 1H), 4.22 − 4.10 (m, 1H), 3.78 − 3.50 (m, 2H), 2.68 (s, 3H), 1.96 − 1.86 (m, 2H), 1.76 − 1.56 (m, 4H), 1.23 − 1.10 (m, 3H), 1.04 − 0.91 (m, 2H) |
| 317 | 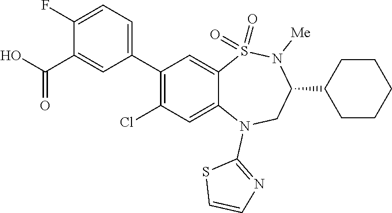 [SMILES: CN1[C@H](C2CCCCC2)CN(c2nccs2)c2cc(Cl)c(-c3ccc(F)c(C(=O)O)c3)cc2S1(=O)=O] | [M + H]⁺ 550.1 | ¹H NMR (300 MHz, DMSO- d₆): δ 13.44 (s, 1H), 8.11 (s, 1H), 8.00 − 7.94 (m, 1H), 7.87 − 7.79 (m, 2H), 7.51 − 7.42 (m, 1H), 7.28 − 7.21 (m, 1H), 6.90 (s, 1H), 4.68 − 4.48 (m, 1H), 3.88 − 3.62 (m, 1H), 3.35 (m, 1H), 2.52 (s, 3H), 2.05 − 1.95 (m, 1H), 1.88 − 1.51 (m, 5H), 1.25 − 1.22 (m, 3H), 1.04 − 0.79 (m, 2H). |
| 318 | 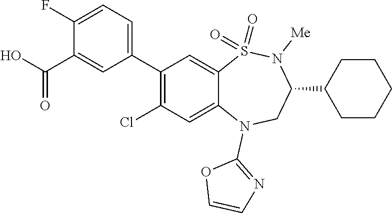 [SMILES: CN1[C@H](C2CCCCC2)CN(c2ncco2)c2cc(Cl)c(-c3ccc(F)c(C(=O)O)c3)cc2S1(=O)=O] | [M + H]⁺ 534.1 | ¹H NMR (300 MHz, DMSO- d₆): δ 13.83 − 13.09 (m, 1H), 8.02 − 7.94 (m, 2H), 7.87 − 7.78 (m, 2H), 7.63 (s, 1H), 7.52 − 7.43 (m, 1H), 6.97 (s, 1H), 4.54 − 4.38 (m, 1H), 3.60 − 3.51 (m, 2H), 2.48 − 2.40 (m, 3H), 2.01 − 1.59 (m, 6H), 1.28 − 1.16 (m, 4H), 1.04 − 0.93 (m, 1H). |

### Ex.319: Synthesis of (R)-5-(7-chloro-3-cyclohexyl-5-(cyclopropylmethyl)-2-methyl-1,1-dioxido-2,3,4,5-tetrahydrobenzo[f][1,2,5]thiadiazepin-8-yl)-2-fluorobenzoic acid

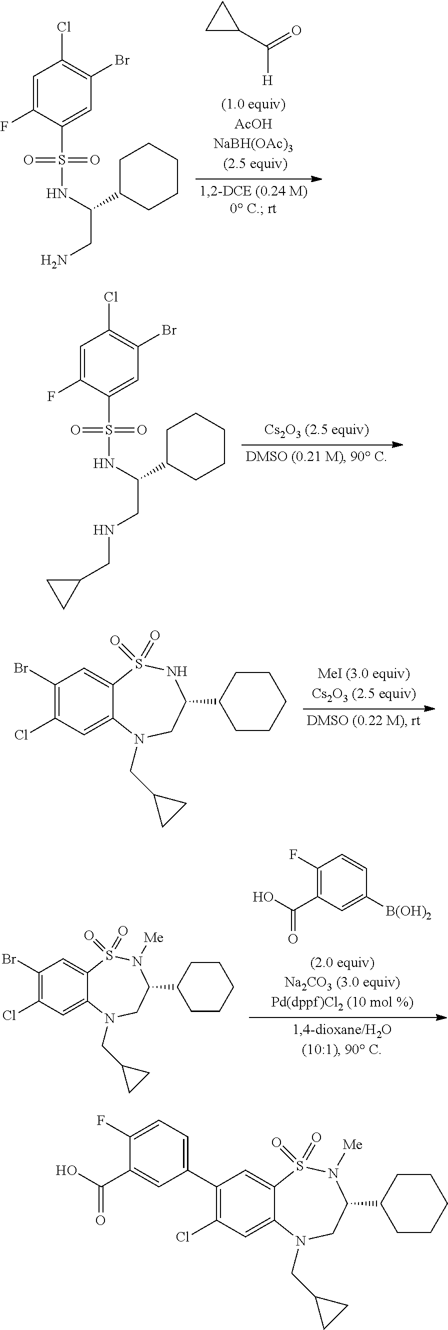

Step 1

To a solution of (R)-N-(2-amino-1-cyclohexylethyl)-5-bromo-4-chloro-2-fluorobenzenesulfonamide (3.0 g, 7.25 mmol, 1.0 eq), cyclopropanecarbaldehyde (510.0 mg, 7.28 mmol, 1.0 eq) and AcOH (0.9 mL) in DCE (30.0 mL) was added NaBH(OAc)₃ (3.9 g, 18.40 mmol, 2.5 eq) at 0° C. under N₂ atmosphere. The reaction mixture was then stirred at room temperature for 2 h. The mixture was poured into water (60 mL) and extracted three times with EtOAc (80 mL each). The combined organic phase was washed three times with brine (80 mL each), dried over Na₂SO₄ and concentrated under reduced pressure to give (R)-5-bromo-4-chloro-N-(1-cyclohexyl-2-((cyclopropylmethyl)amino)ethyl)-2-fluorobenzenesulfonamide (2.0 g, 59% yield) as a colorless oil. ESI MS=467.1 [M+H]⁺. ¹H NMR (300 MHz, CDCl₃): δ 8.13 (d, J=6.0 Hz, 1H), 7.33 (d, J=9.3 Hz, 1H), 3.16-3.06 (m, 1H), 2.77-2.65 (m, 1H), 2.50-2.41 (m, 1H), 2.38-2.23 (m, 2H), 1.78-1.58 (m, 5H), 1.54-1.40 (m, 1H), 1.20-1.03 (m, 3H), 1.00-0.69 (m, 4H), 0.50-0.40 (m, 2H), 0.05 (q, J=4.5 Hz, 2H).

Step 2

A mixture of (R)-5-bromo-4-chloro-N-(1-cyclohexyl-2-((cyclopropylmethyl)amino)ethyl)-2-fluorobenzenesulfonamide (2.0 g, 4.28 mmol, 1.0 eq) and Cs₂CO₃ (3.5 g, 10.74 mmol, 2.5 eq) in DMSO (20.0 mL) was stirred for 2 h at 90° C. under nitrogen atmosphere. The resulting mixture was cooled to room temperature and diluted with H₂O (50 mL). The organic phase was extracted three times with EtOAc (60 mL each). Then the combined organic layers were washed three times with brine (60 mL×3), dried over anhydrous Na₂SO₄ and concentrated under reduced pressure to give (R)-8-bromo-7-chloro-3-cyclohexyl-5-(cyclopropylmethyl)-2,3,4,5-tetrahydrobenzo[f][1,2,5]thiadiazepine 1,1-dioxide (1.5 g, 78% yield) as a yellow solid. ESI MS m/z=447.0 [M+H]⁺.

Step 3

A mixture of (R)-8-bromo-7-chloro-3-cyclohexyl-5-(cyclopropylmethyl)-2,3,4,5-tetrahydrobenzo[f][1,2,5]thiadiazepine 1,1-dioxide (1.5 g, 3.35 mmol, 1.0 eq), Cs₂CO₃ (2.7 g, 8.38 mmol, 2.5 eq) and Mel (1.4 g, 10.05 mmol, 3.0 eq) in DMSO (15.0 mL) was stirred for 1 h at room temperature. The reaction was quenched by the addition of H₂O (40 mL) at room temperature. The resulting mixture was extracted three times with EtOAc (60 mL each). The combined organic layers were then washed three times with brine (60 mL each), dried over anhydrous Na₂SO₄ and concentrated under reduced pressure. The residue was purified by silica gel column chromatography (eluting with EtOAc: Petroleum ether=1:10) to give (R)-8-bromo-7-chloro-3-cyclohexyl-5-(cyclopropylmethyl)-2-methyl-2,3,4,5-tetrahydrobenzo[f][1,2,5]thiadiazepine 1,1-dioxide (800.0 mg, 52% yield) as a white solid. ESI MS m/z=461.1 [M+H]⁺.

Step 4

A mixture of (R)-8-bromo-7-chloro-3-cyclohexyl-5-(cyclopropylmethyl)-2-methyl-2,3,4,5-tetrahydrobenzo[f][1,2,5]thiadiazepine 1,1-dioxide (150.0 mg, 0.33 mmol, 1.0 eq), 5-borono-2-fluorobenzoic acid (119.9 mg, 0.65 mmol, 2.0 eq), Na₂CO₃ (103.0 mg, 0.97 mmol, 3.0 eq) and Pd(dppf)Cl₂ (23.0 mg, 0.03 mmol, 0.1 eq) in 1,4-dioxane (3.0 mL) and H₂O (0.3 mL) were stirred for 1 h at 90° C. under nitrogen atmosphere. The resulting mixture was cooled to room temperature and concentrated under vacuum. The residue was purified by RPHPLC to afford (R)-5-(7-chloro-3-cyclohexyl-5-(cyclopropylmethyl)-2-methyl-1,1-dioxido-2,3,4,5-tetrahydrobenzo[f][1,2,5]thiadiazepin-8-yl)-2-fluorobenzoic acid, Ex.319 (71.2 mg, 42.0%) as a white solid. ESI MS m/z=521.0 [M+H]⁺. ¹H NMR (300 MHz, DMSO-d₆): δ 13.43 (brs, 1H), 7.79-7.73 (m, 1H), 7.61-7.51 (m, 2H), 7.35-7.25 (m, 1H), 7.17 (s, 1H), 3.99-3.84 (m, 1H), 3.80-3.69 (m, 1H), 3.50-3.38 (m, 2H), 3.18-3.08 (m, 1H), 2.85 (s, 3H), 2.07-2.03 (m, 1H), 1.78-1.60 (m, 5H), 1.23-0.95 (m, 6H), 0.60 (d, J=8.1 Hz, 2H), 0.38-0.27 (in, 2H).

Examples 320-325 were prepared using procedures analogous to that used for Ex.319:

| Example | Structure | MS | NMR |
|---|---|---|---|
| 319 | 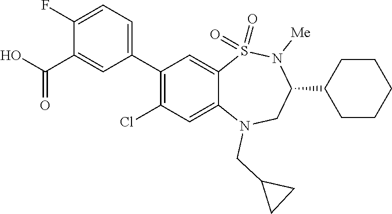 [SMILES: CN1[C@H](C2CCCCC2)CN(CC2CC2)c2cc(Cl)c(-c3ccc(F)c(C(=O)O)c3)cc2S1(=O)=O] | [M + H]⁺ 521.0 | ¹H NMR (300 MHz, DMSO-d₆): δ 13.43 (brs, 1H), 7.79 − 7.73 (m, 1H), 7.61 − 7.51 (m, 2H), 7.35 − 7.25 (m, 1H), 7.17 (s, 1H), 3.99 − 3.84 (m, 1H), 3.80 − 3.69 (m, 1H), 3.50 − 3.38 (m, 2H), 3.18 − 3.08 (m, 1H), 2.85 (s, 3H), 2.07 − 2.03 (m, 1H), 1.78 − 1.60 (m, 5H), 1.23 − 0.95 (m, 6H), 0.60 (d, J = 8.1 Hz, 2H), 0.38 − 0.27 (m, 2H) |
| 320 | 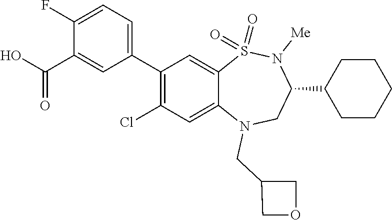 [SMILES: CN1[C@H](C2CCCCC2)CN(CC2COC2)c2cc(Cl)c(-c3ccc(F)c(C(=O)O)c3)cc2S1(=O)=O] | [M + H]⁺ 537.4 | 1H NMR (300 MHz, DMSO-d6): δ 13.47 (s, 1H), 7.82 (d, J = 7.2 Hz, 1H), 7.72 − 7.60 (m, 1H), 7.57 (s, 1H), 7.44 − 7.27 (m, 2H), 4.78 − 4.60 (m, 2H), 4.45 − 4.26 (m, 2H), 4.02 − 3.88 (m, 1H), 3.79 − 3.64 (m, 1H), 3.62 − 3.50 (m, 1H), 3.48 − 3.39 (m, 1H), 3.30 − 3.22 (m, 1H), 3.20 − 3.05 (m, 1H), 2.69 (s, 3H), 2.02 − 1.88 (m, 1H), 1.81 − 1.55 (m, 5H), 1.26 − 0.96 (m, 5H). |
| 321 | 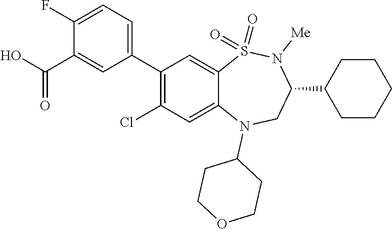 [SMILES: CN1[C@H](C2CCCCC2)CN(C2CCOCC2)c2cc(Cl)c(-c3ccc(F)c(C(=O)O)c3)cc2S1(=O)=O] | [M + H]⁺ 551.1 | ¹H NMR (300 MHz, CDCl₃): δ 8.82 − 8.73 (m, 1H), 7.82 (s, 1H), 7.64 − 7.61 (m, 1H), 7.26 − 7.19 (m, 1H), 6.99 (s, 1H), 4.15 − 4.12 (m, 2H), 3.67 − 3.52 (m, 5H), 3.20 − 2.85 (m, 4H), 2.20 − 2.05 (m, 1H), 1.93 − 1.42 (m, 7H), 1.40 − 1.18 (m, 5H), 1.10 − 0.88 (m, 2H). |
| 322 | 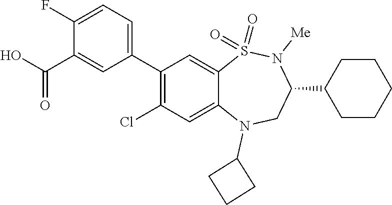 [SMILES: CN1[C@H](C2CCCCC2)CN(C2CCC2)c2cc(Cl)c(-c3ccc(F)c(C(=O)O)c3)cc2S1(=O)=O] | [M + H]⁺ 521.2 | ¹H NMR (400 MHz, DMSO-d₆): δ 13.38 (brs, 1H), 7.82 (d, J = 4.5 Hz, 1H), 7.66 (s, 1H), 7.59 (s, 1H), 7.37 (m, 1H), 7.08 − 6.94 (m, 1H), 4.24 − 4.09 (m, 1H), 3.56 (s, 1H), 3.22 (s, 2H), 2.77 (s, 3H), 2.40 − 2.25 (m, 2H), 2.21 − 2.09 (m, 1H), 2.00 (d, J = 7.9 Hz, 1H), 1.86 − 1.77 (m, 2H), 1.76 − 1.66 (m, 4H), 1.65 − 1.53 (m, 2H), 1.27 − 1.14 (m, 3H), 1.08 − 0.97 (m, 2H) |
| 323 | 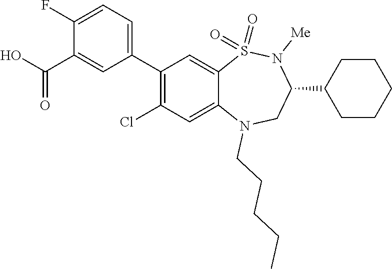 [SMILES: CCCCCN1C[C@@H](C2CCCCC2)N(C)S(=O)(=O)c2cc(-c3ccc(F)c(C(=O)O)c3)c(Cl)cc21] | [M + H]⁺ 537.2 | ¹H NMR (400 MHz, DMSO): δ 13.38 (s, 1H), 7.86 − 7.76 (m, 1H), 7.69 − 7.62 (m, 1H), 7.55 (s, 1H), 7.45 − 7.30 (m, 1H), 7.10 (s, 1H), 4.00 − 3.80 (m, 1H), 3.61 − 3.42 (m, 2H), 3.33 (s, 1H), 3.24 (s, 1H), 2.78 (s, 3H), 2.09 − 1.91 (m, 1H), 1.77 − 1.59 (m, 7H), 1.38 − 1.30 (m, 4H), 1.24 − 1.12 (m, 3H), 1.06 − 0.96 (m, 2H), 0.91 (t, J = 6.8 Hz, 3H) |
| 324 | 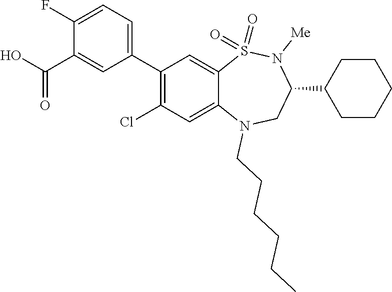 [SMILES: CCCCCCN1C[C@@H](C2CCCCC2)N(C)S(=O)(=O)c2cc(-c3ccc(F)c(C(=O)O)c3)c(Cl)cc21] | [M + H]⁺ 551.2 | ¹H NMR (300 MHz, DMSO-d₆): δ 13.39 (s, 1H), 7.87 − 7.80 (m, 1H), 7.73 − 7.64 (m, 1H), 7.55 (s, 1H), 7.43 − 7.33 (m, 1H), 7.10 (s, 1H), 3.97 − 3.82 (m, 1H), 3.56 − 3.42 (m, 2H), 3.39 − 3.33 (m, 1H), 3.30 − 3.22 (m, 1H), 2.78 (s, 3H), 2.04 − 1.95 (m, 1H), 1.79 − 1.55 (m, 7H), 1.39 − 1.26 (m, 6H), 1.24 − 1.12 (m, 3H), 1.09 − 0.95 (m, 2H), 0.93 − 0.83 (m, 3H). |
| 325 | 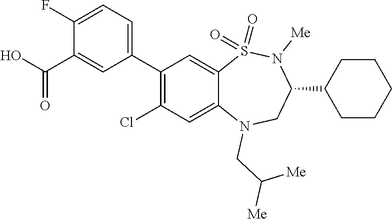 [SMILES: CC(C)CN1C[C@@H](C2CCCCC2)N(C)S(=O)(=O)c2cc(-c3ccc(F)c(C(=O)O)c3)c(Cl)cc21] | [M + H]⁺ 523.1 | ¹H NMR (300 MHz, DMSO-d₆): δ 7.90 − 7.80 (m, 1H), 7.79 − 7.78 (m, 1H), 7.73 − 7.63 (m, 1H), 7.60 (s, 1H), 7.45 − 7.32 (m, 1H), 7.26 (s, 1H), 3.75 − 3.53 (m, 1H), 3.49 − 3.38 (m, 2H), 3.20 − 3.07 (m, 1H), 2.69 (s, 3H), 2.04 − 1.88 (m, 2H), 1.86 − 1.67 (m, 3H), 1.65 − 1.49 (m, 2H), 1.32 − 1.11 (m, 3H), 1.07 − 0.88 (m, 8H). |

### Ex.326: Synthesis of 3-(7-chloro-5-cyclopentyl-3-isobutyl-2-methyl-1,1-dioxido-2,3,4,5-tetrahydrobenzo[f][1,2,5]thiadiazepin-8-yl)benzoic acid

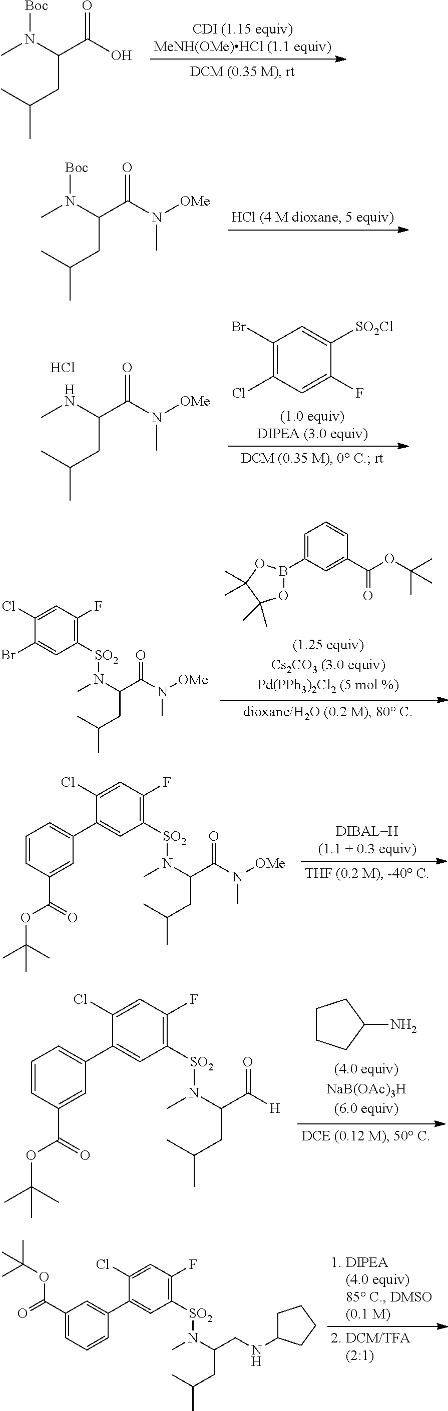

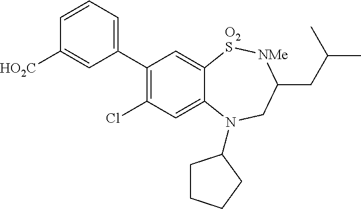 [SMILES: CC(C)CC1CN(C2CCCC2)c2cc(Cl)c(-c3cccc(C(=O)O)c3)cc2S(=O)(=O)N1C]

Step 1

In a 250 mL flask equipped with a stir bar, Boc-N-methyl-dl-leucine (1.5 g, CAS #: 13734-32-2) was dissolved in dichlormethane (17.5 mL, 0.35M). To the resulting solution was added carbonyl diimidazole (1.14 g, 1,15 equiv, CAS #530-62-1). The resulting mixture was stirred for 20 minutes. Then, N, O-di methylhydroxylamine hydrochloride (656.0 mg, 1.1 equiv, CAS #: 6638-79-5) was added. After 6 h, the reaction mixture was concentrated and purified by silica gel column chromatography to afford tert-butyl (1-(methoxy(methyl)amino)-4-methyl-1-oxopentan-2-yl)(methy)carbamate (711.0 mg, 40% yield). ESI MS m/z=311.2 [M-+Na].

Step 2

In a 40 mL vial equipped with a stir bar, tert-butyl (1-(methoxy(methyl)amino)-4-methyl-1-oxopentan-2-yl)(methyl)carbamate (711.0 mg) was combined with hydrochloric acid (4M in dioxane, 5.0 equiv, 3.1 ml. After stirring for 2 h, the reaction mixture was concentrated and the crude N-methoxy-N,4-dimethyl-2(methylamino)pentanamide hydrochloride (554.0 mg, theoretical mass) was used directly without purification. ESI MS m/z=189.2 [M+H]⁺.

Step 3

In a 40 nL vial equipped with a stir bar, N-methoxy-N,4-dimethyl-2-(methylamino)pentanamide hydrochloride (554.0 mg) was suspended in dichloromethane (7.0 mL, 0.35M). The resulting suspension was cooled in an ice and water bath prior to the addition of N,N-diisopropylethylamine (1.29 mL, 3.0 equiv). Next, 5-bromo-4-chloro-2-fluorobenzenesulfonyl chloride (759.0 mg, 1.0 equiv, CAS #: 1070972-67-6) was added to the resulting solution. After stirring for 24 h while being allowed to warm to room temperature, the reaction mixture was concentrated and the residue was purified by silica gel column chromatography to afford 2-((5-bromo-4-chloro-2-fluoro-N-methylphenyl)sulfonamido)-N-methoxy-N,4-dimethylpentanamide (806.6 mg, 71% yield). ESI MS n/z=459.0 [M+1H]⁺.

Step 4

In a 40 mL vial equipped with a stir bar, 2-((5-bromo-4-chloro-2-fluoro-N-methylphenyl)sulfonamido)-N-methoxy-N,4-dimethylpentanamide (806.6 mg), cesium carbonate (1.72 g, 3.0 equiv), tert-butyl 3-(4,4,5,5-tetramethyl-1,3,2-dioxaborolan-2-yl)benzoate (667.0 mg, 1.25 equiv, CAS #: 903895-48-7), and bis(triphenylphosphine)palladium(II) chloride (61.6 mg, 5 mol %, CAS #: 13965-03-2) were combined in a mixture of dioxane (7.6 mL) and water (1.1 mL) under a nitrogen atmosphere. The vial was then sealed with electrical tape and heated at 80° C. for 1 h. Upon cooling to room temperature, the reaction mixture was concentrated and the crude residue was purified by silica gel column chromatography to afford tert-butyl 2′-chloro-4′-fluoro-5′-(N-(1-(methoxy(methyl)amino)-4-methyl-1-oxopentan-2-yl)-N-methylsulfamoyl)-[1,1′-biphenyl]-3-carboxylate (891.7 mg, 91%). ESI MS m/z=501.2 [M-C₄H₈+H]⁺.

Step 5

In a flame-dried 40 mL vial equipped with a stir bar under a nitrogen atmosphere, tert-butyl 2′-chloro-4′-fluoro-5′-(N-(1-(methoxy(methyl)amino)-4-methyl-1-oxopentan-2-yl)-N-methylsulfamoyl)-[1,1′-biphenyl]-3-carboxylate (891.7 mg) was dissolved in tetrahydrofuran (8.0 mL, 0.2M). The resulting solution was then cooled to −40° C. using a dry ice/acetonitrile bath. Next diisobutylaluminum hydride (1M toluene, 1.76 mL, 1.1 equiv, CAS #: 1191-15-7) was added, and the reaction temperature was maintained between −40° C. and −30° C. After 2 h, additional diisobutylaluminum hydride (1 M toluene, 480 μL, 0.3 equiv) was added. The reaction mixture was stirred further between −40° C. and −30° C. After complete consumption of the starting material, as determined by LCMS analysis, a solution of saturated aqueous Rochelle salt (10 mL, CAS #: 6381-59-5) was added to the solution at −30° C. After stirring for 1 h at room temperature, the reaction mixture was further diluted with water and extracted with ethyl acetate. The combined organic layers were dried over magnesium sulfate. Upon concentration, the crude residue was purified by silica gel column chromatography to afford tert-butyl 2′-chloro-4′-fluoro-5′-(N-methyl-N-(4-methyl-1-oxopentan-2-yl)sulfamoyl)-[1,1′-biphenyl]-3-carboxylate (691.8 mg, 87% yield). ESI MS m/z=442.0 [M-C₄H₈+H]⁺.

Step 6

In a 8 mL vial equipped with a stir bar, tert-butyl 2′-chloro-4′-fluoro-5′-(N-methyl-N-(4-methyl-1-oxopentan-2-yl)sulfamoyl)-[1,1′-biphenyl]-3-carboxylate (60 mg) was dissolved in 1,2-dichloroethane (1.0 mL, 0.12M). Next, cyclopentylamine (48 μL, 4.0 equiv, CAS #: 1003-03-8) was added, followed by 1 drop of acetic acid and sodium triacetoxyborohydride (153 mg, 6.0 equiv). The resulting mixture was heated at 55° C. for 1.5 h. Upon cooling to room temperature, the reaction mixture was diluted with saturated aqueous sodium bicarbonate, and the aqueous phase was extracted with dichloromethane. The combined organic layers were passed through a phase separator and concentrated. The crude residue was purified by RPHPLC to afford tert-butyl 2′-chloro-5′-(N-(1-(cyclopentylamino)-4-methylpentan-2-yl)-N-methylsulfamoyl)-4′-fluoro-[1,1′-biphenyl]-3-carboxylate (50.6 mg, 74% yield). [M+H], 567.3.

Step 7 In a 20 mL vial equipped with a stir bar, tert-butyl 2′-chloro-5′-(N-(1-(cyclopentylamino)-4-methylpentan-2-yl)-N-methylsulfamoyl)-4′-fluoro-[1,1′-biphenyl]-3-carboxylate (50.6 mg) was dissolved in dimethyl sulfoxide (890 μL, 0.1M). Next, N,N-diisopropylethylamine (62 μL, 4.0 equiv) was added and the sealed vial was heated at 85° C. for 24 h. Upon cooling to room temperature, the crude reaction mixture was concentrated on a Biotage® V-10 evaporator. The residue was dissolved in dichloromethane (1.0 mL) and trifluoroacetic acid (500 μL) was added. Within 30 minutes, LCMS analysis indicated full conversion to the desired product. The reaction mixture was concentrated, and the crude residue was purified by RPHPLC to afford 3-(7-chloro-5-cyclopentyl-3-isobutyl-2-methyl-1,1-dioxido-2,3,4,5-tetrahydrobenzo[f][1,2,5]thiadiazepin-8-yl)benzoic acid, Ex.326 (4.06 mg, 9% yield). ESI MS m/z=491.2 [M+H]⁺.

Examples 327-331 were prepared using similar procedures as described above for Ex.326:

| Example | Structure | MS | NMR |
|---|---|---|---|
| 326 | 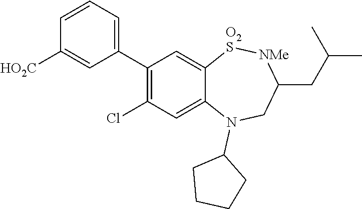 [SMILES: CC(C)CC1CN(C2CCCC2)c2cc(Cl)c(-c3cccc(C(=O)O)c3)cc2S(=O)(=O)N1C] | [M + H]⁺ 491.2 | |
| 327 | 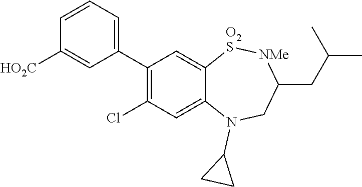 [SMILES: CC(C)CC1CN(C2CC2)c2cc(Cl)c(-c3cccc(C(=O)O)c3)cc2S(=O)(=O)N1C] | [M + H]⁺ 463.2 | |
| 328 | 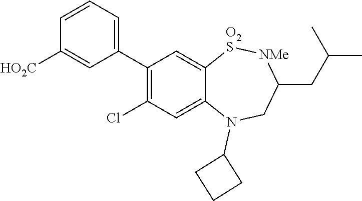 [SMILES: CC(C)CC1CN(C2CCC2)c2cc(Cl)c(-c3cccc(C(=O)O)c3)cc2S(=O)(=O)N1C] | [M + H]⁺ 477.2 | |
| 329 | 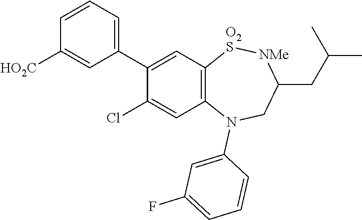 [SMILES: CC(C)CC1CN(c2cccc(F)c2)c2cc(Cl)c(-c3cccc(C(=O)O)c3)cc2S(=O)(=O)N1C] | [M + H]⁺ 517.2 | |
| 330 | 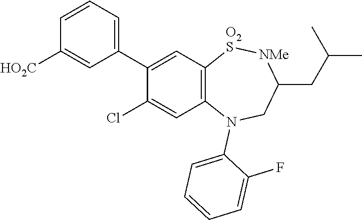 [SMILES: CC(C)CC1CN(c2ccccc2F)c2cc(Cl)c(-c3cccc(C(=O)O)c3)cc2S(=O)(=O)N1C] | [M + H]⁺ 517.2 | |
| 331 | 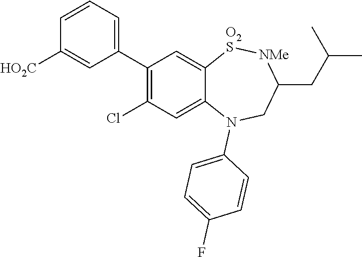 [SMILES: CC(C)CC1CN(c2ccc(F)cc2)c2cc(Cl)c(-c3cccc(C(=O)O)c3)cc2S(=O)(=O)N1C] | [M + H]⁺ 517.2 | ¹H NMR (400 MHz, Acetonitrile- d₃) δ 8.16 − 8.02 (m, 2H), 7.85 (s, 1H), 7.78 − 7.70 (m, 1H), 7.60 (t, J = 7.7 Hz, 1H), 7.27 (s, 1H), 7.10 − 7.01 (m, 2H), 7.01 − 6.88 (m, 2H), 3.97 − 3.93 (m, 2H), 3.55 − 3.48 (m, 1H), 2.65 (s, 3H), 1.79 − 1.69 (m, 1H), 1.64 − 1.57 (m, 1H), 0.95 − 0.91 (m, 6H) |

### Ex.332: Synthesis of(R)-5-(7-(benzyloxy)-3-cyclohexyl-2-methyl-1,1-dioxido-5-phenyl-2,3,4,5-tetrahydrobenzo[f][1,2,5]thiadiazepin-8-yl)-2-fluorobenzoic acid

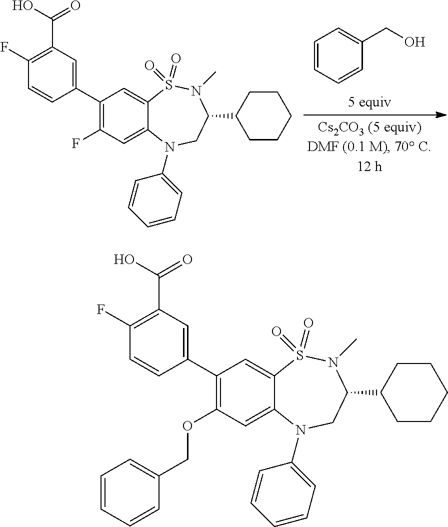

In a 2 mL vial equipped with a stir bar, (R)-5-(3-cyclohexyl-7-fluoro-2-methyl-1,1-dioxido-5-phenyl-2,3,4,5-tetrahydrobenzo[f][1,2,5]thiadiazepin-8-yl)-2-fluorobenzoic acid (15 mg, 1.0 equiv.) was dissolved in DMF (0.285 ml), followed by addition of benzyl alcohol (15.4 mg, 5.0 equiv., CAS #100-51-6) and cesium carbonate (46.4 mg, 5.0 equiv.) at room temperature. Then, the reaction mixture was stirred 12 h at 70° C. in a heating block. Reaction progress was monitored through LC-MS. After 12 h, the reaction was quenched with formic acid (0.2 mL) and concentrated under vacuum. The crude residue was re-dissolved in 2.0 mL of dimethyl sulfoxide, passed through a 0.45 μm syringe filter, and purified by RPHPLC to afford (R)-5-(7-(benzyloxy)-3-cyclohexyl-2-methyl-1,1-dioxido-5-phenyl-2,3,4,5-tetrahydrobenzo[f][1,2,5]thiadiazepin-8-yl)-2-fluorobenzoic acid, Ex.332 (12.0 mg, 68% yield). ESI MS m/z=615.3 [M+H]⁺.

| Example | Structure | MS | NMR |
|---|---|---|---|
| 332 | 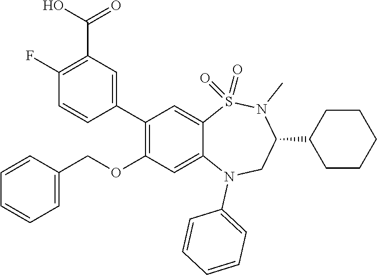 [SMILES: CN1[C@H](C2CCCCC2)CN(c2ccccc2)c2cc(OCc3ccccc3)c(-c3ccc(F)c(C(=O)O)c3)cc2S1(=O)=O] | [M + H]⁺ 615.3 | ¹H NMR (500 MHz, DMSO-d6) δ 13.35 (br, 1H), 8.07 (dd, J = 7.0, 2.5 Hz, 1H), 7.85 − 7.78 (m, 1H), 7.71 (s, 1H), 7.36 (t, J = 9.0 Hz, 1H), 7.33 − 7.25 (m, 5H), 7.21 (t, J = 7.8 Hz, 2H), 7.05 (s, 1H), 6.92 − 6.69 (m, 3H), 5.12 (q, J = 12.3 Hz, 2H), 4.35 (d, J = 15.7 Hz, 1H), 2.57 (s, 3H), 2.10 − 1.50 (m, 6H), 1.34 − 0.84 (m, 5H). |

The following compounds were prepared using a procedure analogous to that described for Ex.332:

| Example | Structure | MS | NMR |
|---|---|---|---|
| 333 | 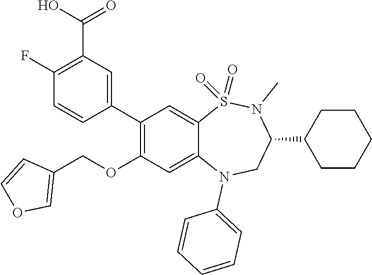 [SMILES: CN1[C@H](C2CCCCC2)CN(c2ccccc2)c2cc(OCc3ccoc3)c(-c3ccc(F)c(C(=O)O)c3)cc2S1(=O)=O] | [M + H]⁺ 605.1 | ¹H NMR (500 MHz, DMSO-d6) δ 13.41 (br, 1H), 8.02 (d, J = 5.5 Hz, 1H), 7.81 - 7.75 (m, 1H), 7.70 (s, 1H), 7.62 (t, J = 1.4 Hz, 1H), 7.57 (s, 1H), 7.35 (t, J = 9.6 Hz, 1H), 7.24 (t, J = 7.7 Hz, 2H), 7.07 (s, 1H), 6.89 - 6.75 (m, 3H), 6.47 - 6.44 (m, 1H), 4.99 (t, J = 8.3 Hz, 2H), 4.36 (d, J = 15.6 Hz, 1H), 3.51 - 3.36 (m, 2H), 2.57 (s, 3H), 2.10 - 1.99 (m, 1H), 1.93 - 1.46 (m, 5H), 1.31 - 0.89 (m, 5H). |
| 334 | 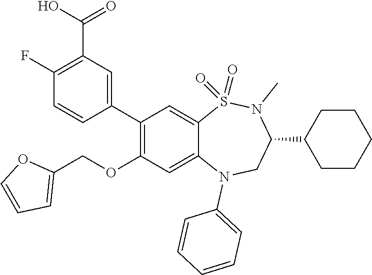 [SMILES: CN1[C@H](C2CCCCC2)CN(c2ccccc2)c2cc(OCc3ccco3)c(-c3ccc(F)c(C(=O)O)c3)cc2S1(=O)=O] | [M + H]⁺ 605.2 | |
| 335 | 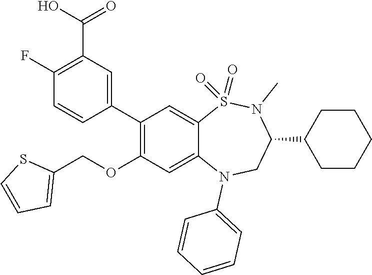 [SMILES: CN1[C@H](C2CCCCC2)CN(c2ccccc2)c2cc(OCc3cccs3)c(-c3ccc(F)c(C(=O)O)c3)cc2S1(=O)=O] | [M + H]⁺ 621.0 | |
| 336 | 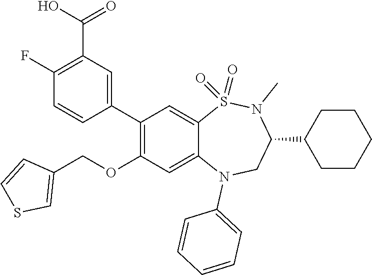 [SMILES: CN1[C@H](C2CCCCC2)CN(c2ccccc2)c2cc(OCc3ccsc3)c(-c3ccc(F)c(C(=O)O)c3)cc2S1(=O)=O] | [M + H]⁺ 621.1 | |
| 337 | 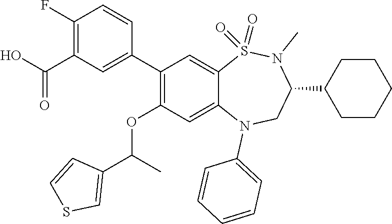 [SMILES: CC(Oc1cc2c(cc1-c1ccc(F)c(C(=O)O)c1)S(=O)(=O)N(C)[C@H](C1CCCCC1)CN2c1ccccc1)c1ccsc1] | [M + H]⁺ 635.2 | |
| 338 | 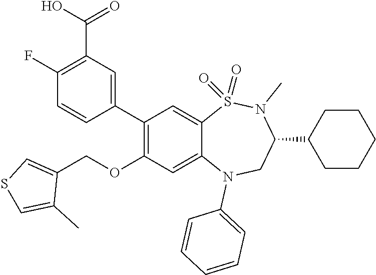 [SMILES: Cc1cscc1COc1cc2c(cc1-c1ccc(F)c(C(=O)O)c1)S(=O)(=O)N(C)[C@H](C1CCCCC1)CN2c1ccccc1] | [M + H]⁺ 635.2 | |
| 339 | 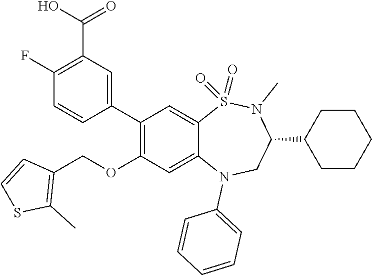 [SMILES: Cc1sccc1COc1cc2c(cc1-c1ccc(F)c(C(=O)O)c1)S(=O)(=O)N(C)[C@H](C1CCCCC1)CN2c1ccccc1] | [M + H]⁺ 635.2 | |
| 340 | 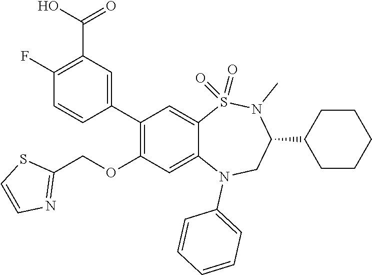 [SMILES: CN1[C@H](C2CCCCC2)CN(c2ccccc2)c2cc(OCc3nccs3)c(-c3ccc(F)c(C(=O)O)c3)cc2S1(=O)=O] | [M + H]⁺ 622.2 | |
| 341 | 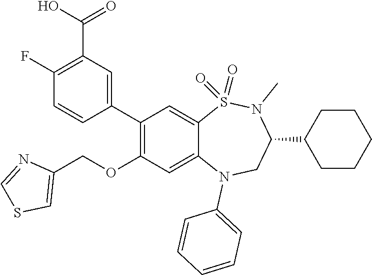 [SMILES: CN1[C@H](C2CCCCC2)CN(c2ccccc2)c2cc(OCc3cscn3)c(-c3ccc(F)c(C(=O)O)c3)cc2S1(=O)=O] | [M + H]⁺ 622.2 | |
| 342 | 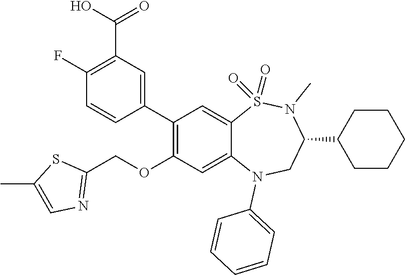 [SMILES: Cc1cnc(COc2cc3c(cc2-c2ccc(F)c(C(=O)O)c2)S(=O)(=O)N(C)[C@H](C2CCCCC2)CN3c2ccccc2)s1] | [M + H]⁺ 636.2 | |
| 343 | 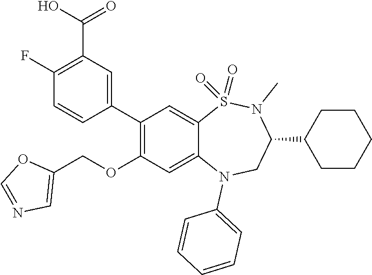 [SMILES: CN1[C@H](C2CCCCC2)CN(c2ccccc2)c2cc(OCc3cnco3)c(-c3ccc(F)c(C(=O)O)c3)cc2S1(=O)=O] | [M + H]⁺ 606.2 | |
| 344 | 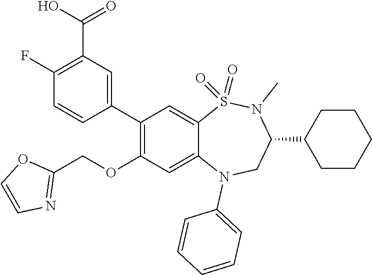 [SMILES: CN1[C@H](C2CCCCC2)CN(c2ccccc2)c2cc(OCc3ncco3)c(-c3ccc(F)c(C(=O)O)c3)cc2S1(=O)=O] | [M + H]⁺ 606.2 | |
| 345 | 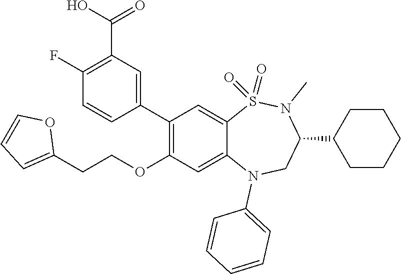 [SMILES: CN1[C@H](C2CCCCC2)CN(c2ccccc2)c2cc(OCCc3ccco3)c(-c3ccc(F)c(C(=O)O)c3)cc2S1(=O)=O] | [M + H]⁺ 619.2 | |
| 346 | 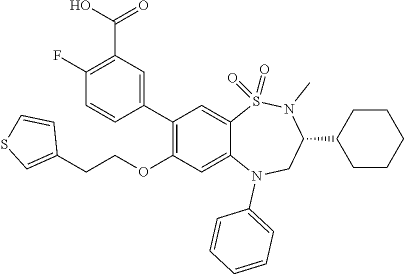 [SMILES: CN1[C@H](C2CCCCC2)CN(c2ccccc2)c2cc(OCCc3ccsc3)c(-c3ccc(F)c(C(=O)O)c3)cc2S1(=O)=O] | [M + H]⁺ 635.2 | |
| 347 | 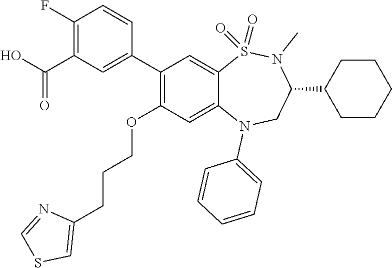 [SMILES: CN1[C@H](C2CCCCC2)CN(c2ccccc2)c2cc(OCCCc3cscn3)c(-c3ccc(F)c(C(=O)O)c3)cc2S1(=O)=O] | [M + H]⁺ 650.3 | |
| 348 | 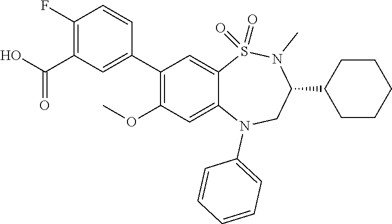 [SMILES: COc1cc2c(cc1-c1ccc(F)c(C(=O)O)c1)S(=O)(=O)N(C)[C@H](C1CCCCC1)CN2c1ccccc1] | [M + H]⁺ 539.1 | |
| 349 | 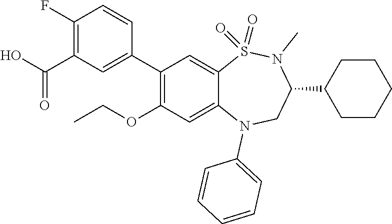 [SMILES: CCOc1cc2c(cc1-c1ccc(F)c(C(=O)O)c1)S(=O)(=O)N(C)[C@H](C1CCCCC1)CN2c1ccccc1] | [M + H]⁺ 553.2 | |
| 350 | 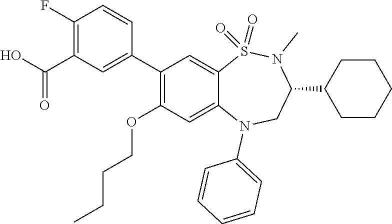 [SMILES: CCCCOc1cc2c(cc1-c1ccc(F)c(C(=O)O)c1)S(=O)(=O)N(C)[C@H](C1CCCCC1)CN2c1ccccc1] | [M + H]⁺ 581.3 | |
| 351 | 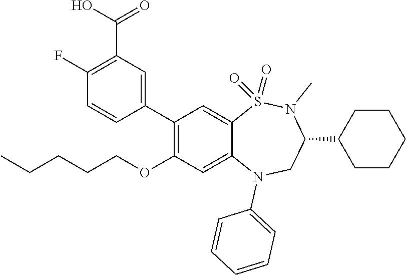 [SMILES: CCCCCOc1cc2c(cc1-c1ccc(F)c(C(=O)O)c1)S(=O)(=O)N(C)[C@H](C1CCCCC1)CN2c1ccccc1] | [M + H]⁺ 595.3 | |
| 352 | 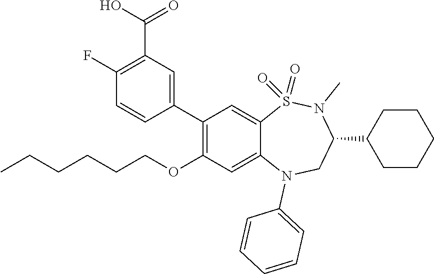 [SMILES: CCCCCCOc1cc2c(cc1-c1ccc(F)c(C(=O)O)c1)S(=O)(=O)N(C)[C@H](C1CCCCC1)CN2c1ccccc1] | [M + H]⁺ 609.3 | |
| 353 | 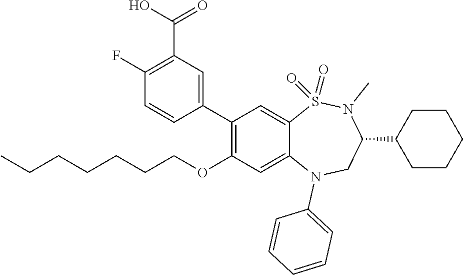 [SMILES: CCCCCCCOc1cc2c(cc1-c1ccc(F)c(C(=O)O)c1)S(=O)(=O)N(C)[C@H](C1CCCCC1)CN2c1ccccc1] | [M + H]⁺ 623.4 | |
| 354 | 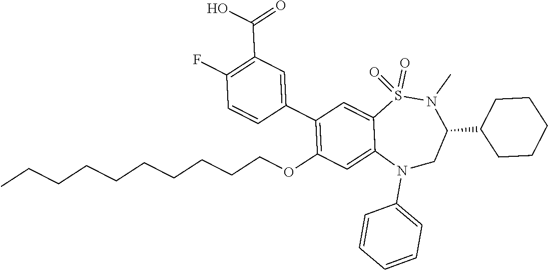 [SMILES: CCCCCCCCCCOc1cc2c(cc1-c1ccc(F)c(C(=O)O)c1)S(=O)(=O)N(C)[C@H](C1CCCCC1)CN2c1ccccc1] | [M + H]⁺ 665.2 | |
| 355 | 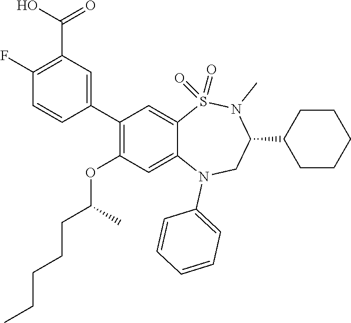 [SMILES: CCCCC[C@@H](C)Oc1cc2c(cc1-c1ccc(F)c(C(=O)O)c1)S(=O)(=O)N(C)[C@H](C1CCCCC1)CN2c1ccccc1] | [M + H]⁺ 623.3 | |
| 356 | 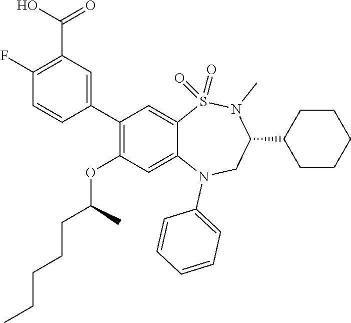 [SMILES: CCCCC[C@H](C)Oc1cc2c(cc1-c1ccc(F)c(C(=O)O)c1)S(=O)(=O)N(C)[C@H](C1CCCCC1)CN2c1ccccc1] | [M + H]⁺ 623.3 | |
| 357 | 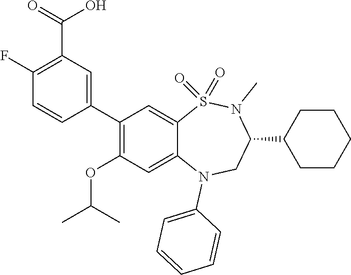 [SMILES: CC(C)Oc1cc2c(cc1-c1ccc(F)c(C(=O)O)c1)S(=O)(=O)N(C)[C@H](C1CCCCC1)CN2c1ccccc1] | [M + H]⁺ 567.3 | |
| 358 | 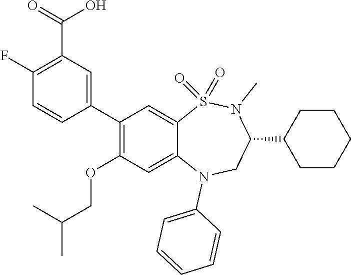 [SMILES: CC(C)COc1cc2c(cc1-c1ccc(F)c(C(=O)O)c1)S(=O)(=O)N(C)[C@H](C1CCCCC1)CN2c1ccccc1] | [M + H]⁺ 581.4 | |
| 359 | 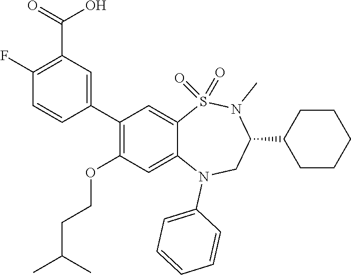 [SMILES: CC(C)CCOc1cc2c(cc1-c1ccc(F)c(C(=O)O)c1)S(=O)(=O)N(C)[C@H](C1CCCCC1)CN2c1ccccc1] | [M + H]⁺ 595.4 | |
| 360 | 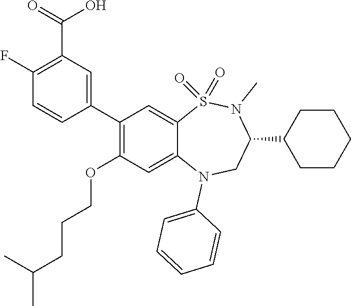 [SMILES: CC(C)CCCOc1cc2c(cc1-c1ccc(F)c(C(=O)O)c1)S(=O)(=O)N(C)[C@H](C1CCCCC1)CN2c1ccccc1] | [M + H]⁺ 609.4 | |
| 361 | 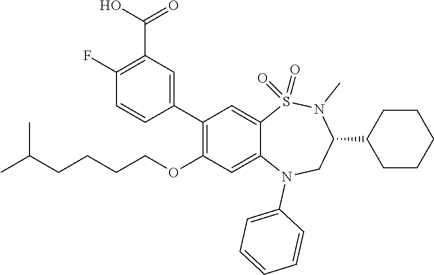 [SMILES: CC(C)CCCCOc1cc2c(cc1-c1ccc(F)c(C(=O)O)c1)S(=O)(=O)N(C)[C@H](C1CCCCC1)CN2c1ccccc1] | [M + H]⁺ 623.3 | |
| 362 | 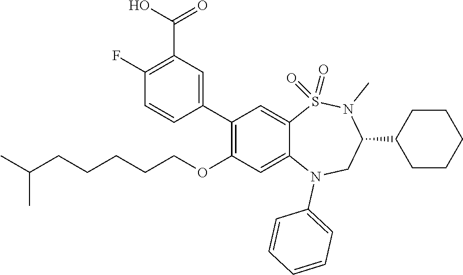 [SMILES: CC(C)CCCCCOc1cc2c(cc1-c1ccc(F)c(C(=O)O)c1)S(=O)(=O)N(C)[C@H](C1CCCCC1)CN2c1ccccc1] | [M + H]⁺ 637.4 | |
| 363 | 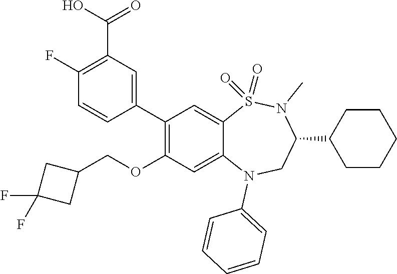 [SMILES: CN1[C@H](C2CCCCC2)CN(c2ccccc2)c2cc(OCC3CC(F)(F)C3)c(-c3ccc(F)c(C(=O)O)c3)cc2S1(=O)=O] | [M + H]⁺ 629.1 | |
| 364 | 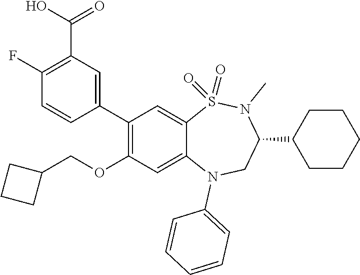 [SMILES: CN1[C@H](C2CCCCC2)CN(c2ccccc2)c2cc(OCC3CCC3)c(-c3ccc(F)c(C(=O)O)c3)cc2S1(=O)=O] | [M + H]⁺ 593.1 | |
| 365 | 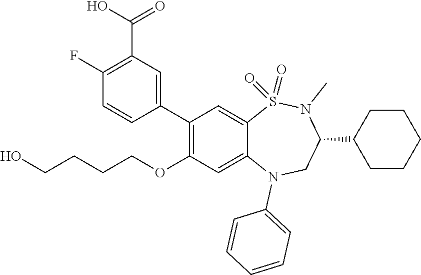 [SMILES: CN1[C@H](C2CCCCC2)CN(c2ccccc2)c2cc(OCCCCO)c(-c3ccc(F)c(C(=O)O)c3)cc2S1(=O)=O] | [M + H]⁺ 597.2 | |
| 366 | 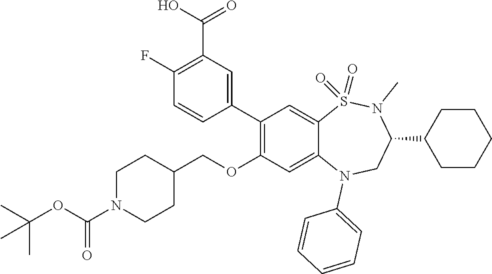 [SMILES: CN1[C@H](C2CCCCC2)CN(c2ccccc2)c2cc(OCC3CCN(C(=O)OC(C)(C)C)CC3)c(-c3ccc(F)c(C(=O)O)c3)cc2S1(=O)=O] | [M + H]⁺ 722.2 | |
| 367 | 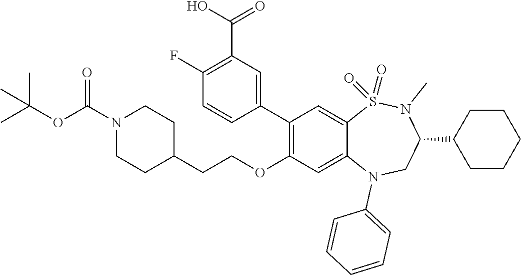 [SMILES: CN1[C@H](C2CCCCC2)CN(c2ccccc2)c2cc(OCCC3CCN(C(=O)OC(C)(C)C)CC3)c(-c3ccc(F)c(C(=O)O)c3)cc2S1(=O)=O] | [M + H]⁺ 736.8 | |
| 368 | 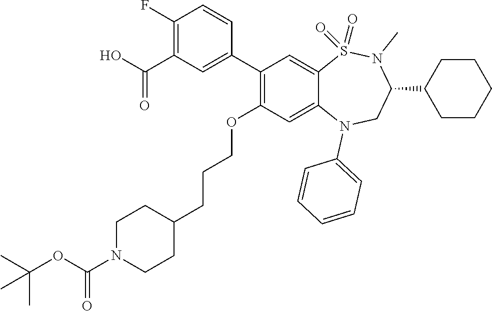 [SMILES: CN1[C@H](C2CCCCC2)CN(c2ccccc2)c2cc(OCCCC3CCN(C(=O)OC(C)(C)C)CC3)c(-c3ccc(F)c(C(=O)O)c3)cc2S1(=O)=O] | [M − H]⁻ 748.7 | |
| 369 | 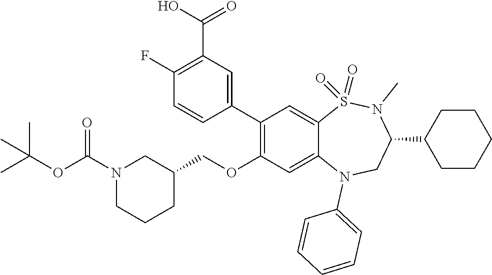 [SMILES: CN1[C@H](C2CCCCC2)CN(c2ccccc2)c2cc(OC[C@@H]3CCCN(C(=O)OC(C)(C)C)C3)c(-c3ccc(F)c(C(=O)O)c3)cc2S1(=O)=O] | [M − H]⁻ 720.3 | |
| 370 | 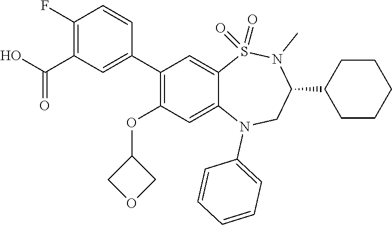 [SMILES: CN1[C@H](C2CCCCC2)CN(c2ccccc2)c2cc(OC3COC3)c(-c3ccc(F)c(C(=O)O)c3)cc2S1(=O)=O] | [M + H]⁺ 581.3 | |
| 371 | 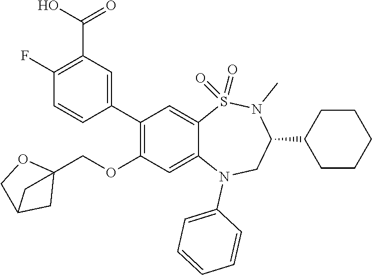 [SMILES: CN1[C@H](C2CCCCC2)CN(c2ccccc2)c2cc(OCC34CC(CO3)C4)c(-c3ccc(F)c(C(=O)O)c3)cc2S1(=O)=O] | [M + H]⁺ 621.2 | |
| 372 | 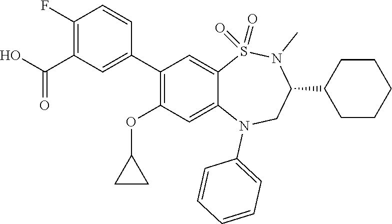 [SMILES: CN1[C@H](C2CCCCC2)CN(c2ccccc2)c2cc(OC3CC3)c(-c3ccc(F)c(C(=O)O)c3)cc2S1(=O)=O] | [M + H]⁺ 565.3 | |
| 373 | 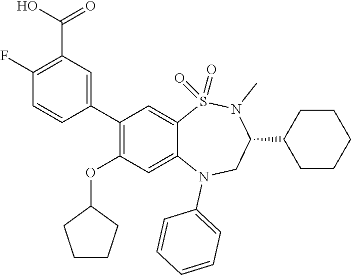 [SMILES: CN1[C@H](C2CCCCC2)CN(c2ccccc2)c2cc(OC3CCCC3)c(-c3ccc(F)c(C(=O)O)c3)cc2S1(=O)=O] | [M + H]⁺ 593.3 | |
| 374 | 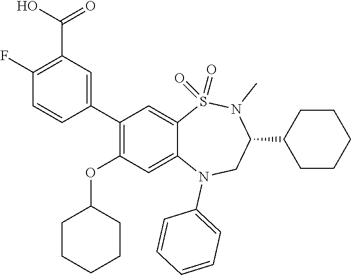 [SMILES: CN1[C@H](C2CCCCC2)CN(c2ccccc2)c2cc(OC3CCCCC3)c(-c3ccc(F)c(C(=O)O)c3)cc2S1(=O)=O] | [M + H]⁺ 607.4 | |
| 375 | 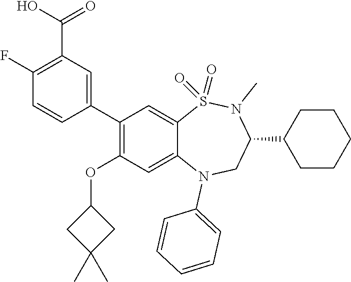 [SMILES: CN1[C@H](C2CCCCC2)CN(c2ccccc2)c2cc(OC3CC(C)(C)C3)c(-c3ccc(F)c(C(=O)O)c3)cc2S1(=O)=O] | [M + H]⁺ 607.3 | |
| 376 | 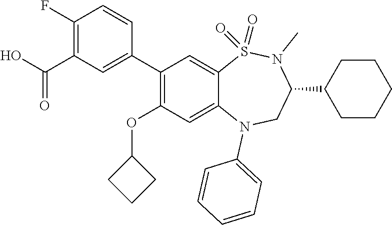 [SMILES: CN1[C@H](C2CCCCC2)CN(c2ccccc2)c2cc(OC3CCC3)c(-c3ccc(F)c(C(=O)O)c3)cc2S1(=O)=O] | [M + H]⁺ 579.2 | |
| 377 | 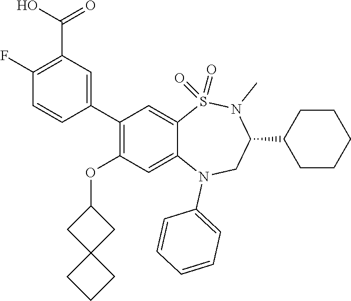 [SMILES: CN1[C@H](C2CCCCC2)CN(c2ccccc2)c2cc(OC3CC4(CCC4)C3)c(-c3ccc(F)c(C(=O)O)c3)cc2S1(=O)=O] | [M + H]⁺ 619.2 | |
| 378 | 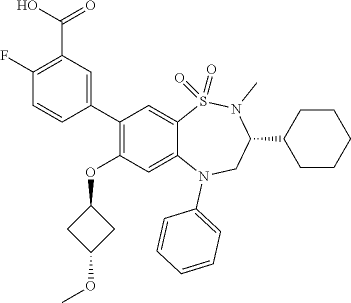 [SMILES: CO[C@H]1C[C@H](Oc2cc3c(cc2-c2ccc(F)c(C(=O)O)c2)S(=O)(=O)N(C)[C@H](C2CCCCC2)CN3c2ccccc2)C1] | [M + H]⁺ 609.2 | |
| 379 | 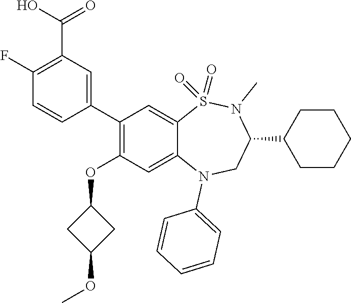 [SMILES: CO[C@H]1C[C@@H](Oc2cc3c(cc2-c2ccc(F)c(C(=O)O)c2)S(=O)(=O)N(C)[C@H](C2CCCCC2)CN3c2ccccc2)C1] | [M + H]⁺ 609.2 | |
| 380 | 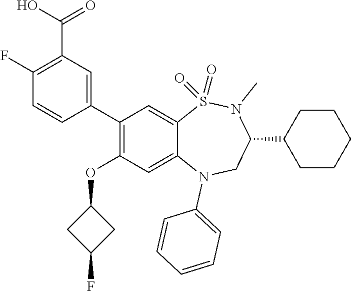 [SMILES: CN1[C@H](C2CCCCC2)CN(c2ccccc2)c2cc(O[C@H]3C[C@@H](F)C3)c(-c3ccc(F)c(C(=O)O)c3)cc2S1(=O)=O] | [M + H]⁺ 597.3 | |
| 381 | 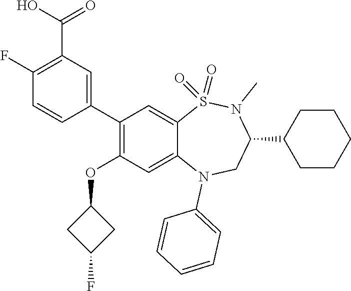 [SMILES: CN1[C@H](C2CCCCC2)CN(c2ccccc2)c2cc(O[C@H]3C[C@H](F)C3)c(-c3ccc(F)c(C(=O)O)c3)cc2S1(=O)=O] | [M + H]⁺ 597.3 | |
| 382 | 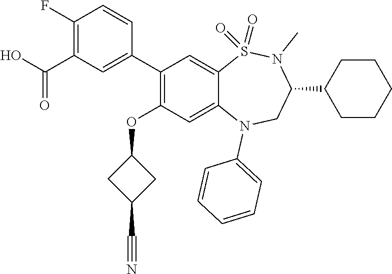 [SMILES: CN1[C@H](C2CCCCC2)CN(c2ccccc2)c2cc(O[C@H]3C[C@@H](C#N)C3)c(-c3ccc(F)c(C(=O)O)c3)cc2S1(=O)=O] | [M + H]⁺ 604.2 | |
| 383 | 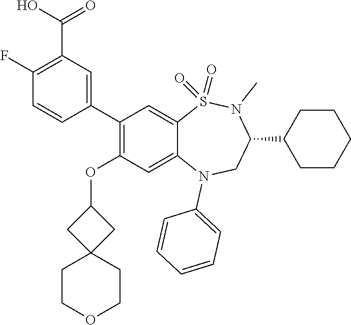 [SMILES: CN1[C@H](C2CCCCC2)CN(c2ccccc2)c2cc(OC3CC4(CCOCC4)C3)c(-c3ccc(F)c(C(=O)O)c3)cc2S1(=O)=O] | [M + H]⁺ 649.2 | |
| 384 | 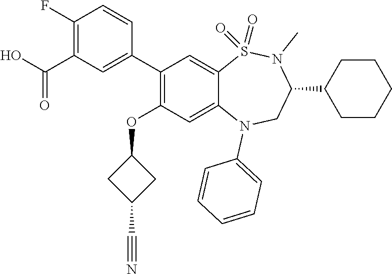 [SMILES: CN1[C@H](C2CCCCC2)CN(c2ccccc2)c2cc(O[C@H]3C[C@H](C#N)C3)c(-c3ccc(F)c(C(=O)O)c3)cc2S1(=O)=O] | [M + H]⁺ 604.2 | |
| 385 | 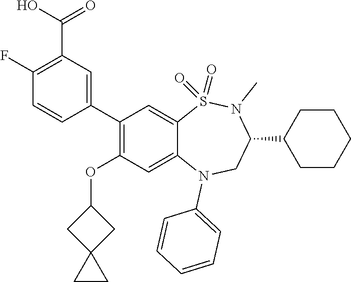 [SMILES: CN1[C@H](C2CCCCC2)CN(c2ccccc2)c2cc(OC3CC4(CC4)C3)c(-c3ccc(F)c(C(=O)O)c3)cc2S1(=O)=O] | [M + H]⁺ 605.3 | |
| 386 | 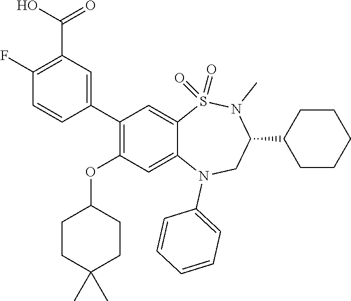 [SMILES: CN1[C@H](C2CCCCC2)CN(c2ccccc2)c2cc(OC3CCC(C)(C)CC3)c(-c3ccc(F)c(C(=O)O)c3)cc2S1(=O)=O] | [M + H]⁺ 635.2 | |
| 387 | 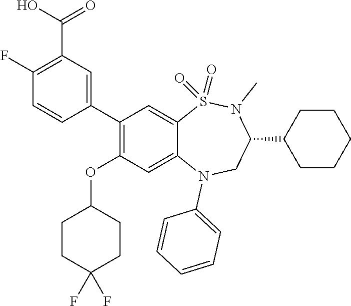 | [M + H]⁺ 635.2 | |
| 388 | 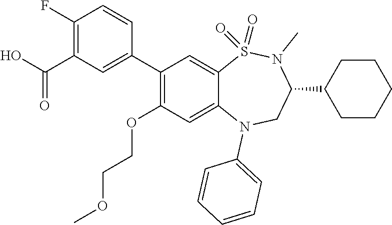 [SMILES: COCCOc1cc2c(cc1-c1ccc(F)c(C(=O)O)c1)S(=O)(=O)N(C)[C@H](C1CCCCC1)CN2c1ccccc1] | [M + H]⁺ 583.3 | |

### Ex.389: Synthesis of(R)-5-(3-cyclohexyl-2-methyl-7-((1-methyl-1H-imidazol-4-yl)methoxy)-1,1-dioxido-5-phenyl-2,3,4,5-tetrahydrobenzo[f][1,2,5]thiadiazepin-8-yl)-2-fluorobenzoic acid

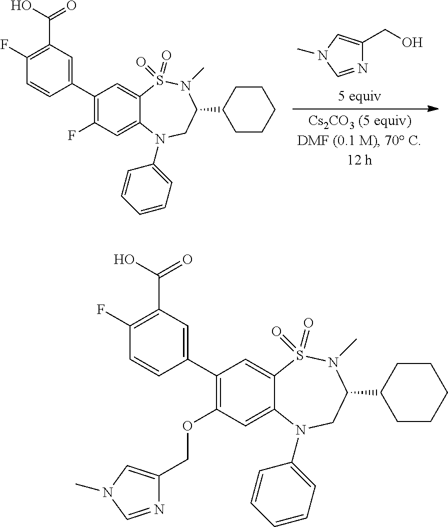

In a 2 mL vial equipped with a stir bar, (R)-5-(3-cyclohexyl-7-fluoro-2-methyl-1,1-dioxido-5-phenyl-2,3,4,5-tetrahydrobenzo[f][1,2,5]thiadiazepin-8-yl)-2-fluorobenzoic acid (15 mg, 1.0 equiv.) was dissolved in dry DMF (0.285 ml) and subsequently added 1-Methyl-1H-imidazole-4-methanol (16.0 mg, 5.0 equiv., CAS #17289-25-7) and cesium carbonate (46.4 mg, 5.0 equiv.). The reaction mixture was stirred at 70° C. in a heating blockand reaction progress was monitored through LC-MS. After 12 h, the reaction was quenched with formic acid (0.2 mL) and concentrated under vacuum. The crude residue was re-dissolved in 2.0 mL of dimethyl sulfoxide, purified through reversed phase C18 flash column chromatography (acetonitrile/water) to afford (R)-5-(3-cyclohexyl-2-methyl-7-((1-methyl-1H-imidazol-4-yl)methoxy)-1,1-dioxido-5-phenyl-2,3,4,5-tetrahydrobenzo[f][1,2,5]thiadiazepin-8-yl)-2-fluorobenzoic acid, Ex.389 (14.5 mg, 82% yield). ESI MS m/z=619.4 [M+H]⁺.

| Example | Structure | MS | NMR |
|---|---|---|---|
| 389 | 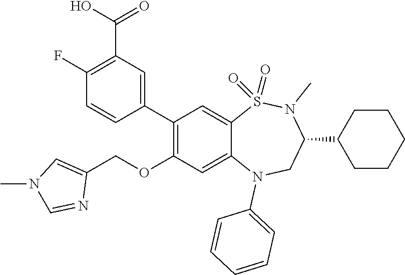 [SMILES: CN1[C@H](C2CCCCC2)CN(c2ccccc2)c2cc(OCc3cn(C)cn3)c(-c3ccc(F)c(C(=O)O)c3)cc2S1(=O)=O] | [M + H]⁺ 619.4 | ¹H NMR (400 MHz, DMSO-d6) δ 7.97 (dd, J = 7.0, 2.5 Hz, 1H), 7.86 − 7.78 (m, 1H), 7.67 (s, 1H), 7.49 (s, 1H), 7.34 (dd, J = 10.6, 8.6 Hz, 1H), 7.24 (t, J = 7.7 Hz, 2H), 7.17 (s, 1H), 6.99 (s, 1H), 6.89 − 6.78 (m, 3H), 4.95 (s, 2H), 4.35 (d, J = 15.4 Hz, 1H), 3.59 (s, 3H), 2.57 (s, 3H), 2.10 − 1.48 (m, 6H), 1.34 − 0.85 (m, 5H). |

The following compounds were prepared using a procedure analogous to that described for Ex.389.

| Example | Structure | MS |
|---|---|---|
| 390 | 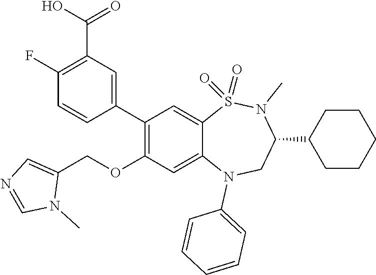 [SMILES: CN1[C@H](C2CCCCC2)CN(c2ccccc2)c2cc(OCc3cncn3C)c(-c3ccc(F)c(C(=O)O)c3)cc2S1(=O)=O] | [M + H]⁺ 619.4 |
| 391 | 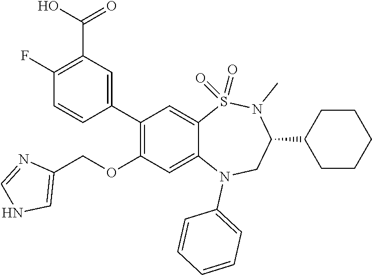 [SMILES: CN1[C@H](C2CCCCC2)CN(c2ccccc2)c2cc(OCc3c[nH]cn3)c(-c3ccc(F)c(C(=O)O)c3)cc2S1(=O)=O] | [M + H]⁺ 605.3 |
| 392 | 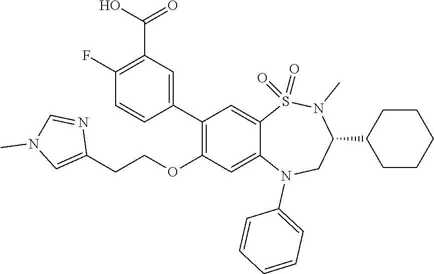 [SMILES: CN1[C@H](C2CCCCC2)CN(c2ccccc2)c2cc(OCCc3cn(C)cn3)c(-c3ccc(F)c(C(=O)O)c3)cc2S1(=O)=O] | [M + H]⁺ 633.4 |

### Ex.393: Synthesis of (R)-5-(7-((1-acetylpiperidin-4-yl)oxy)-3-cyclohexyl-2-methyl-1,1-dioxido-5-phenyl-2,3,4,5-tetrahydrobenzo[f][1,2,5]thiadiazepin-8-yl)-2-fluorobenzoic acid

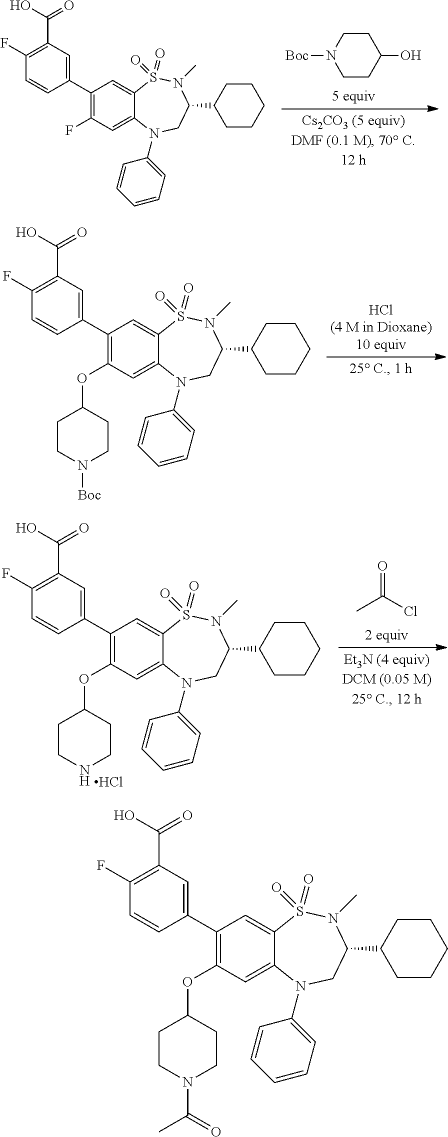

Step 1

In a 8 mL vial equipped with a stir bar, (R)-5-(3-cyclohexyl-7-fluoro-2-methyl-1,1-dioxido-5-phenyl-2,3,4,5-tetrahydrobenzo[f][1,2,5]thiadiazepin-8-yl)-2-fluorobenzoic acid (100.0 mg, 1.0 equiv.) was dissolved in DMF (1.9 mL), followed by addition of tert-butyl 4-hydroxypiperidine-1-carboxylate (191.0 mg, 5.0 equiv., CAS #109384-19-2) and cesium carbonate (309.0 mg, 5.0 equiv.). The reaction mixture was stirred at 70° C. in a heating block. After 12 h, the reaction was quenched with formic acid (1.0 mL) and concentrated under vacuum. The crude residue was loaded into a 100 g gold C18 column and purified through reversed phase flash column chromatography (acetonitrile/water) to afford (R)-5-(7-((1-(tert-butoxycarbonyl)piperidin-4-yl)oxy)-3-cyclohexyl-2-methyl-1,1-dioxido-5-phenyl-2,3,4,5-tetrahydrobenzo[f][1,2,5]thiadiazepin-8-yl)-2-fluorobenzoic acid (108.0 mg, 80% yield). ESI MS m/z=707.1 [M+H]⁺.

Step 2

In a 2 mL vial equipped with a stir bar, (R)-5-(7-((1-(tert-butoxycarbonyl)piperidin-4-yl)oxy)-3-cyclohexyl-2-methyl-1,1-dioxido-5-phenyl-2,3,4,5-tetrahydrobenzo[f][1,2,5]thiadiazepin-8-yl)-2 fluorobenzoic acid (100.0 mg, 1.0 equiv.) was taken and added HCl (4M in Dioxane, 0.353 mL, 10.0 equiv., CAS #7647-01-0). The reaction mixture was stirred for 1 h at 25° C. Crude was concentrated under vacuum to afford (R)-5-(3-cyclohexyl-2-methyl-1,1-dioxido-5-phenyl-7-(piperidin-4-yloxy)-2,3,4,5-tetrahydrobenzo[f][1,2,5]thiadiazepin-8-yl)-2-fluorobenzoic acid. ESI MS m/z=608.5 [M+H]⁺. Crude was transferred to the next step without further purification.

Step 3

In a 4 mL vial equipped with a stir bar, (R)-5-(3-cyclohexyl-2-methyl-1,1-dioxido-5-phenyl-7-(piperidin-4-yloxy)-2,3,4,5-tetrahydrobenzo[f][1,2,5]thiadiazepin-8-yl)-2-fluorobenzoic acid (12.5 mg, 1.0 equiv.) was dissolved in dry DCM (0.4 mL) under nitrogen and followed by addition of triethylamine (11.47 μl, 4.0 equiv.). Then, the reaction mixture was cooled down to zero degree and added acetyl chloride (3.23 mg, 2.0 equiv., CAS #75-36-5). After addition, reaction mixture was stirred at 25° C. After 12 h, reaction mixture was concentrated under vacuum and the crude residue was re-dissolved in 2.0 mL of dimethyl sulfoxide, passed through a 0.45 μm syringe filter, and purified by RPTIPLC to afford (R)-5-(7-((1-acetylpiperidin-4-yl)oxy)-3-cyclohexyl-2-methyl-1,1-dioxido-5-phenyl-2,3,4,5-tetrahydrobenzo[f][1,2,5]thiadiazepin-8-yl)-2-fluorobenzoic acid, Ex.393 (4.4 mg, 330 yield). ESI MS m/z=650.2 [M+H]⁺.

| Example | Structure | MS | NMR |
|---|---|---|---|
| 393 | 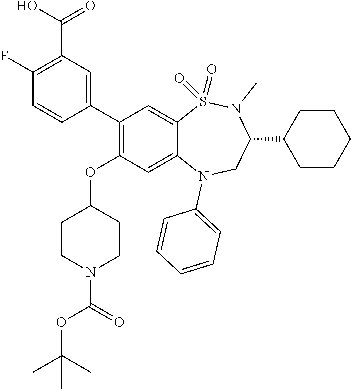 [SMILES: CN1[C@H](C2CCCCC2)CN(c2ccccc2)c2cc(OC3CCN(C(=O)OC(C)(C)C)CC3)c(-c3ccc(F)c(C(=O)O)c3)cc2S1(=O)=O] | [M + H]⁺ 707.1 | ¹H NMR (400 MHz, DMSO-d6) δ 13.32 (s, 1H), 8.06 (dd, J = 7.1, 2.5 Hz, 1H), 7.89 − 7.78 (m, 1H), 7.73 (s, 1H), 7.40 (dd, J = 10.7, 8.7 Hz, 1H), 7.25 (t, J = 7.8 Hz, 2H), 6.98 (s, 1H), 6.88 − 6.78 (m, 3H), 4.62 (s, 1H), 4.35 (d, J = 15.4 Hz, 1H), 3.58 − 3.18 (m, 6H), 2.57 (s, 3H), 2.06 − 1.42 (m, 10H), 1.36 (s, 9H), 1.31 − 0.87 (m, 5H). |
| 394 | 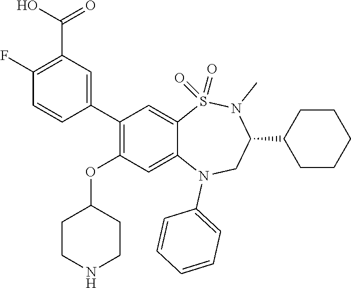 [SMILES: CN1[C@H](C2CCCCC2)CN(c2ccccc2)c2cc(OC3CCNCC3)c(-c3ccc(F)c(C(=O)O)c3)cc2S1(=O)=O] | [M + H]⁺ 608.5 | |
| 395 | 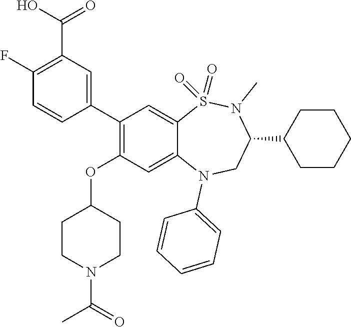 [SMILES: CC(=O)N1CCC(Oc2cc3c(cc2-c2ccc(F)c(C(=O)O)c2)S(=O)(=O)N(C)[C@H](C2CCCCC2)CN3c2ccccc2)CC1] | [M + H]⁺ 650.2 | ¹H NMR (400 MHz, DMSO-d6) δ 8.08 − 7.97 (m, 1H), 7.83 − 7.75 (m, 1H), 7.73 (d, J = 2.8 Hz, 1H), 7.35 (t, J = 9.6 Hz, 1H), 7.29 − 7.19 (m, 2H), 7.05 − 6.75 (m, 4H), 4.67 (s, 1H), 4.35 (d, J = 15.4 Hz, 1H), 2.57 (d, J = 4.7 Hz, 3H), 2.10 − 2.01 (m, 1H), 1.95 (s, 3H), 1.92 − 1.44 (m, 9H), 1.34 − 0.87 (m, 5H). |

The following compounds were prepared using a procedure analogous to that described for Ex.393.

| Example | Structure | MS |
|---|---|---|
| 396 | 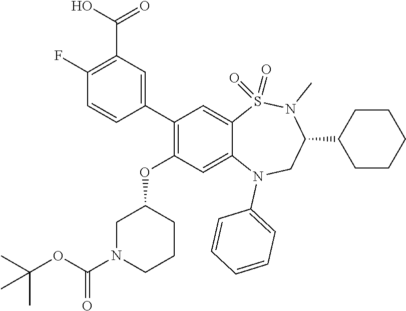 [SMILES: CN1[C@H](C2CCCCC2)CN(c2ccccc2)c2cc(O[C@@H]3CCCN(C(=O)OC(C)(C)C)C3)c(-c3ccc(F)c(C(=O)O)c3)cc2S1(=O)=O] | [M + H]⁺ 707.3 |
| 397 | 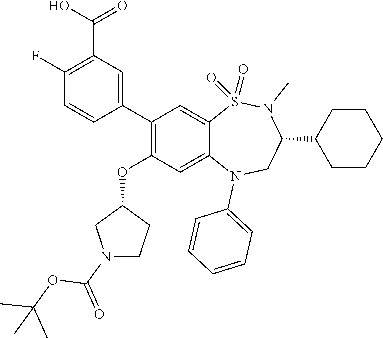 [SMILES: CN1[C@H](C2CCCCC2)CN(c2ccccc2)c2cc(O[C@@H]3CCN(C(=O)OC(C)(C)C)C3)c(-c3ccc(F)c(C(=O)O)c3)cc2S1(=O)=O] | [M − H]⁻ 692.5 |
| 398 | 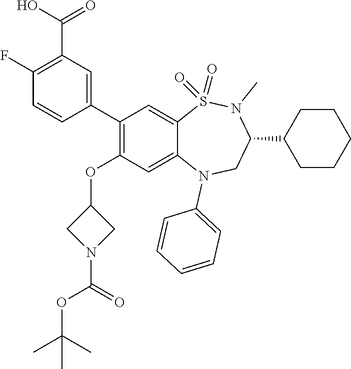 [SMILES: CN1[C@H](C2CCCCC2)CN(c2ccccc2)c2cc(OC3CN(C(=O)OC(C)(C)C)C3)c(-c3ccc(F)c(C(=O)O)c3)cc2S1(=O)=O] | [M − H]⁻ 678.6 |
| 399 | 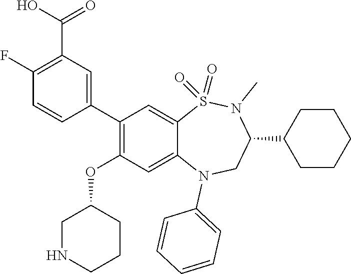 [SMILES: CN1[C@H](C2CCCCC2)CN(c2ccccc2)c2cc(O[C@@H]3CCCNC3)c(-c3ccc(F)c(C(=O)O)c3)cc2S1(=O)=O] | [M + H]⁺ 608.4 |

### Ex.400: Synthesis of (R)-5-(3-cyclohexyl-7-((1-(isopropylcarbamoyl)piperidin-4-yl)oxy)-2-methyl-1,1-dioxido-5-phenyl-2,3,4,5-tetrahydrobenzo[f][1,2,5]thiadiazepin-8-yl)-2-fluorobenzoic acid

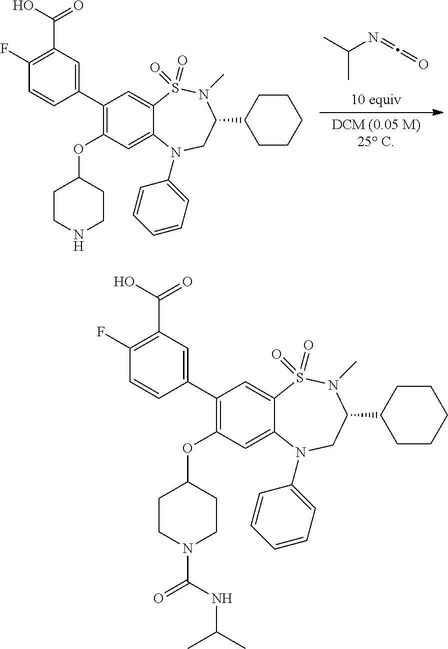

In a 4 mL vial equipped with a stir bar, (R)-5-(3-cyclohexyl-2-methyl-1,1-dioxido-5-phenyl-7-(piperidin-4-yloxy)-2,3,4,5-tetrahydrobenzo[f][1,2,5]thiadiazepin-8-yl)-2-fluorobenzoic acid (12.5 mg, 1.0 equiv.) was dissolved in dry DCM (0.4 mL) under nitrogen. Reaction mixture was cooled down to 0° C. and was added 2-isocyanatopropane (8.75 mg, 5.0 equiv.). Then, the reaction vial was placed at room temp and stirred for 12 h. At this time, LCMS analysis indicated incomplete conversion of starting material. Added 5 equiv. of 2-isocyanatopropane (8.75 mg) and stirred for additional 12 h. Reaction progress was monitored through LC-MS. After completion, reaction mixture was concentrated under vacuum and the crude residue was re-dissolved in 2.0 mL of dimethyl sulfoxide, passed through a 0.45 μm syringe filter, and purified by RPHPLC to afford (R)-5-(3-cyclohexyl-7-((1-(isopropylcarbamoyl)piperidin-4-yl)oxy)-2-methyl-1,1-dioxido-5-phenyl-2,3,4,5-tetrahydrobenzo[f][1,2,5]thiadiazepin-8-yl)-2-fluorobenzoic acid, Ex.400 (3.8 mg, 27% yield). ESI MS m/z=693.4 [M+H]⁺.

| Example | Structure | MS | NMR |
|---|---|---|---|
| 400 | 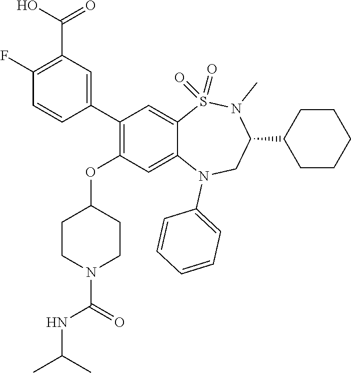 [SMILES: CC(C)NC(=O)N1CCC(Oc2cc3c(cc2-c2ccc(F)c(C(=O)O)c2)S(=O)(=O)N(C)[C@H](C2CCCCC2)CN3c2ccccc2)CC1] | [M + H]⁺ 693.4 | ¹H NMR (400 MHz, DMSO-d6) δ 8.05 − 7.95 (m, 1H), 7.81 − 7.74 (m, 1H), 7.72 (s, 1H), 7.35 (t, J = 9.5 Hz, 1H), 7.24 (t, J = 7.8 Hz, 2H), 6.97 (s, 1H), 6.88 − 6.76 (m, 3H), 6.11 (d, J = 7.6 Hz, 1H), 4.59 (s, 1H), 4.35 (d, J = 15.4 Hz, 1H), 3.70 (h, J = 6.8 Hz, 2H), 2.57 (s, 3H), 2.13 − 2.07 (m, 15H), 1.01 (d, J = 6.7 Hz, 6H). |

### Ex. 401: Synthesis of (R)-5-(3-cyclohexyl-7-(2-fluorophenoxy)-2-methyl-1,1-dioxido-5-phenyl-2,3,4,5-tetrahydrobenzo[f][1,2,5]thiadiazepin-8-yl)-2-fluorobenzoic acid

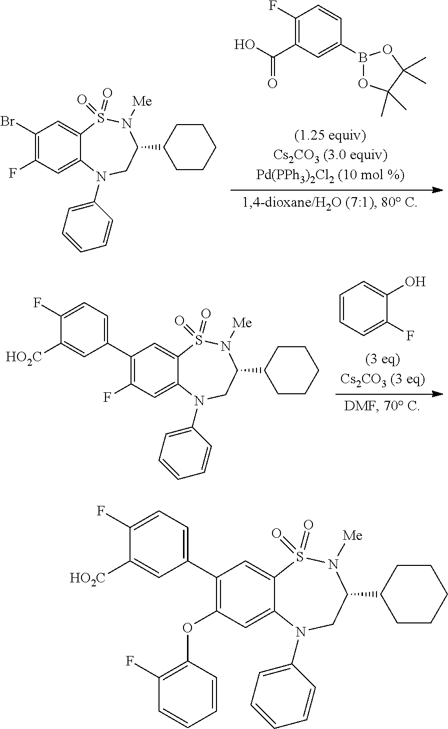

Step 1

To a flask equipped with a stir bar and (R)-8-bromo-3-cyclohexyl-7-fluoro-2-methyl-5-phenyl-2,3,4,5-tetrahydrobenzo[f][1,2,5]thiadiazepine 1,1-dioxide (1 g, 2.140 mmol), 2-fluoro-5-(4,4,5,5-tetramethyl-1,3,2-dioxaborolan-2-yl)benzoic acid (0.712 g, 2.67 mmol), cesium carbonate (2.091 g, 6.42 mmol), and PdCl₂(dppf) (0.157 g, 0.214 mmol) was added 1,4-dioxane (18.60 mL) and water (2.79 mL). The reaction was sealed and heated at 80° C. for 15 hours. Upon complete conversion, the reaction was diluted with EtOAc and acidified with 1 N HCl. The aqueous layer was extracted with EtOAc and the combined organic layers were dried over Na₂SO₄, filtered, and concentrated under reduced pressure. The residue was then purified by silica gel column chromatography (eluent: 0-25% EtOAc/cyclohexane) to afford (R)-5-(3-cyclohexyl-7-fluoro-2-methyl-1,1-dioxido-5-phenyl-2,3,4,5-tetrahydrobenzo[f][1,2,5]thiadiazepin-8-yl)-2-fluorobenzoic acid (986 mg, 1.872 mmol, 88% yield). ¹H NMR (400 MHz, CDCl₃) δ 8.22 (d, J=6.8 Hz, 1H), 8.01 (d, J=8.5 Hz, 1H), 7.81-7.72 (m, 1H), 7.40 (t, J=7.9 Hz, 2H), 7.27 (t, J=10.3 Hz, 1H), 7.18-7.10 (m, 3H), 6.74 (d, J=12.4 Hz, 1H), 4.36-4.28 (m, 1H), 4.25-4.20 (m, 1H), 3.23 (s, 1H), 2.94 (s, 3H), 1.85-1.70 (m, 5H), 1.35-1.24 (m, 2H), 1.22-1.11 (m, 2H), 0.97 (dd, J=11. 1, 4.4 Hz, 2H). ESI MS m/z=527.3[M+H]⁺.

Step 2

To a vial containing, cesium carbonate (30.0 mg, 0.092 mmol), and (R)-2-chloro-5-(3-cyclohexyl-7-fluoro-2-methyl-1,1-dioxido-5-phenyl-2,3,4,5-tetrahydrobenzo[f][1,2,5]thiadiazepin-8-yl)benzoic acid (10 mg, 0.018 mmol) in DMF (0.184 mL) was added 2-fluorophenol (6.19 mg, 0.055 mmol). The reaction was then heated to 70° C. After complete conversion (˜2 hours), the reaction was quenched with formic acid (50 μL) and directly purified via preparative HIPLC to afford (R)-2-chloro-5-(3-cyclohexyl-7-(2-fluorophenoxy)-2-methyl-1,1-dioxido-5-phenyl-2,3,4,5-tetrahydrobenzo[f][1,2,5]thiadiazepin-8-yl)benzoic acid, Ex.401 (3 mg, 4.72.mol, 25.600 yield. ¹H NMR (400 MHz, DMSO) δ 13.51 (s, 1H), 8.03-7.95 (m, 1H), 7.85 (s, 1H), 7.80-7.74 (m, 1H), 7.66-7.60 (m, 1H), 7.34-7.24 (m, 2H), 7.24-7.12 (m, 4H), 6.96-6.83 (m, 3H), 6.40 (s, 1H), 4.37-4.26 (m, 2H), 3.73-3.60 (m, 1H), 2.68 (s, 3H), 1.95-1.86 (m, 2H), 1.75-1.66 (m, 2H), 1.65-1.52 (m, 2H), 1.26-1.08 (m, 3H), 1.01-0.88 (in, 2H). ESI MS m/z=635.4 [M+H]⁺.

Examples 402-465 were made using similar methods to those described above for Ex.401:

| Example | Structure | MS | NMR |
|---|---|---|---|
| 401 | 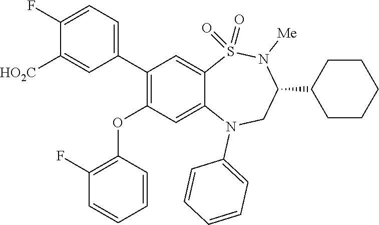 [SMILES: CN1[C@H](C2CCCCC2)CN(c2ccccc2)c2cc(Oc3ccccc3F)c(-c3ccc(F)c(C(=O)O)c3)cc2S1(=O)=O] | [M + H]⁺ 635.4 | ¹H NMR (400 MHz, DMSO) δ 13.51 (s, 1H), 8.03-7.95 (m, 1H), 7.85 (s, 1H), 7.80-7.74 (m, 1H), 7.66-7.60 (m, 1H), 7.34-7.24 (m, 2H), 7.24- 7.12 (m, 4H), 6.96-6.83 (m, 3H), 6.40 (s, 1H), 4.37-4.26 (m, 2H), 3.73-3.60 (m, 1H), 2.68 (s, 3H), 1.95-1.86 (m, 2H), 1.75-1.66 (m, 2H), 1.65-1.52 (m, 2H), 1.26-1.08 (m, 3H), 1.01-0.88 (m, 2H). |
| 402 | 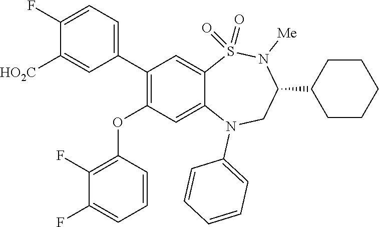 [SMILES: CN1[C@H](C2CCCCC2)CN(c2ccccc2)c2cc(Oc3cccc(F)c3F)c(-c3ccc(F)c(C(=O)O)c3)cc2S1(=O)=O] | [M + H]⁺ 637.3 | |
| 403 | 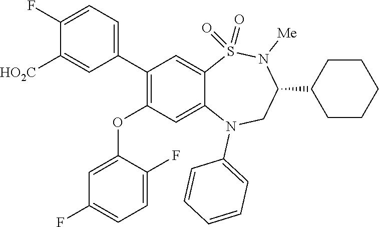 [SMILES: CN1[C@H](C2CCCCC2)CN(c2ccccc2)c2cc(Oc3cc(F)ccc3F)c(-c3ccc(F)c(C(=O)O)c3)cc2S1(=O)=O] | [M + H]⁺ 637.3 | |
| 404 | 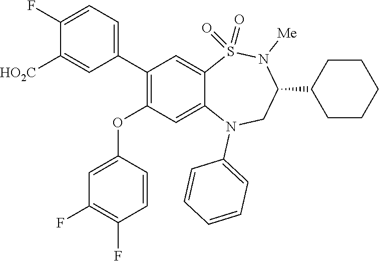 [SMILES: CN1[C@H](C2CCCCC2)CN(c2ccccc2)c2cc(Oc3ccc(F)c(F)c3)c(-c3ccc(F)c(C(=O)O)c3)cc2S1(=O)=O] | [M + H]⁺ 637.4 | |
| 405 | 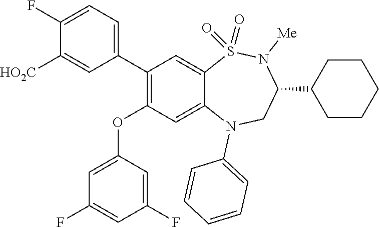 [SMILES: CN1[C@H](C2CCCCC2)CN(c2ccccc2)c2cc(Oc3cc(F)cc(F)c3)c(-c3ccc(F)c(C(=O)O)c3)cc2S1(=O)=O] | [M + H]⁺ 637.4 | |
| 406 | 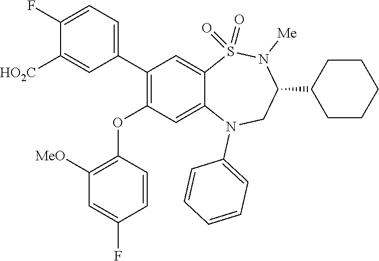 [SMILES: COc1cc(F)ccc1Oc1cc2c(cc1-c1ccc(F)c(C(=O)O)c1)S(=O)(=O)N(C)[C@H](C1CCCCC1)CN2c1ccccc1] | [M + H]⁺ 649.5 | |
| 407 | 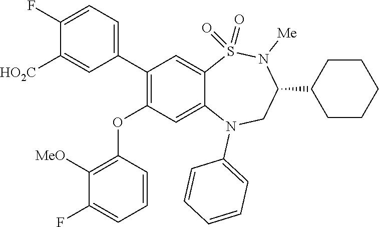 [SMILES: COc1c(F)cccc1Oc1cc2c(cc1-c1ccc(F)c(C(=O)O)c1)S(=O)(=O)N(C)[C@H](C1CCCCC1)CN2c1ccccc1] | [M + H]⁺ 649.5 | |
| 408 | 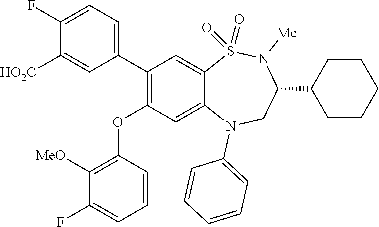 [SMILES: COc1c(F)cccc1Oc1cc2c(cc1-c1ccc(F)c(C(=O)O)c1)S(=O)(=O)N(C)[C@H](C1CCCCC1)CN2c1ccccc1] | [M + H]⁺ 649.5 | |
| 409 | 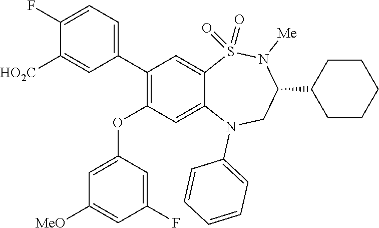 [SMILES: COc1cc(F)cc(Oc2cc3c(cc2-c2ccc(F)c(C(=O)O)c2)S(=O)(=O)N(C)[C@H](C2CCCCC2)CN3c2ccccc2)c1] | [M + H]⁺ 649.5 | |
| 410 | 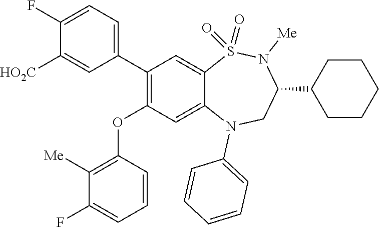 [SMILES: Cc1c(F)cccc1Oc1cc2c(cc1-c1ccc(F)c(C(=O)O)c1)S(=O)(=O)N(C)[C@H](C1CCCCC1)CN2c1ccccc1] | [M + H]⁺ 633.6 | |
| 411 | 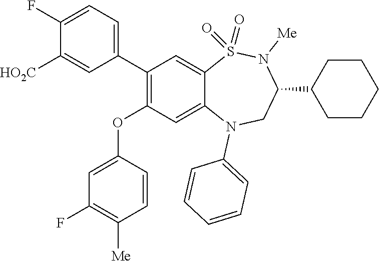 [SMILES: Cc1ccc(Oc2cc3c(cc2-c2ccc(F)c(C(=O)O)c2)S(=O)(=O)N(C)[C@H](C2CCCCC2)CN3c2ccccc2)cc1F] | [M + H]⁺ 633.6 | |
| 412 | 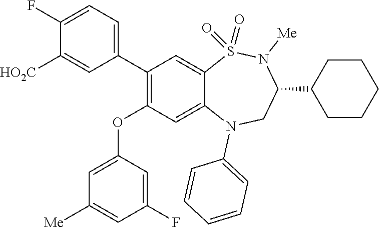 [SMILES: Cc1cc(F)cc(Oc2cc3c(cc2-c2ccc(F)c(C(=O)O)c2)S(=O)(=O)N(C)[C@H](C2CCCCC2)CN3c2ccccc2)c1] | [M + H]⁺ 633.4 | |
| 413 | 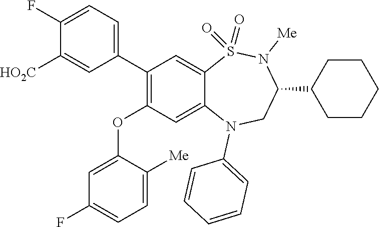 [SMILES: Cc1ccc(F)cc1Oc1cc2c(cc1-c1ccc(F)c(C(=O)O)c1)S(=O)(=O)N(C)[C@H](C1CCCCC1)CN2c1ccccc1] | [M + H]⁺ 633.6 | |
| 414 | 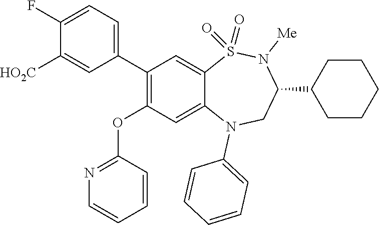 [SMILES: CN1[C@H](C2CCCCC2)CN(c2ccccc2)c2cc(Oc3ccccn3)c(-c3ccc(F)c(C(=O)O)c3)cc2S1(=O)=O] | [M + H]⁺ 602.5 | |
| 415 | 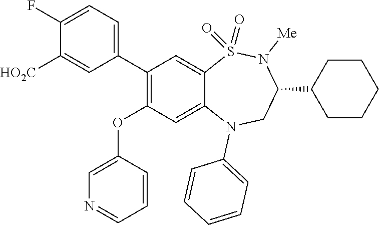 [SMILES: CN1[C@H](C2CCCCC2)CN(c2ccccc2)c2cc(Oc3cccnc3)c(-c3ccc(F)c(C(=O)O)c3)cc2S1(=O)=O] | [M + H]⁺ 602.5 | |
| 416 | 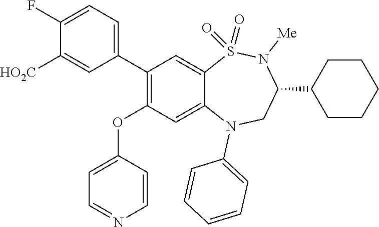 [SMILES: CN1[C@H](C2CCCCC2)CN(c2ccccc2)c2cc(Oc3ccncc3)c(-c3ccc(F)c(C(=O)O)c3)cc2S1(=O)=O] | [M + H]⁺ 602.5 | |
| 417 | 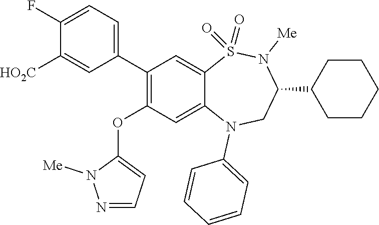 [SMILES: CN1[C@H](C2CCCCC2)CN(c2ccccc2)c2cc(Oc3ccnn3C)c(-c3ccc(F)c(C(=O)O)c3)cc2S1(=O)=O] | [M + H]⁺ 592.4 | |
| 418 | 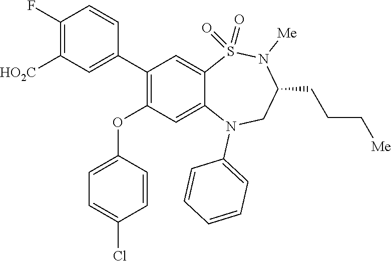 [SMILES: CCCC[C@@H]1CN(c2ccccc2)c2cc(Oc3ccc(Cl)cc3)c(-c3ccc(F)c(C(=O)O)c3)cc2S(=O)(=O)N1C] | [M + H]⁺ 609.2 | ¹H NMR (400 MHz, DMSO-d₆) δ 13.38 (s, 1H), 8.05 (dd, J = 7.1, 2.5 Hz, 1H), 7.91-7.82 (m, 2H), 7.45- 7.33 (m, 3H), 7.20 (t, J = 8.0 Hz, 2H), 7.13 (d, J = 8.9 Hz, 2H), 6.89- 6.79 (m, 3H), 6.67 (s, 1H), 4.09 (d, J = 15.8 Hz, 1H), 3.79-3.67 (m, 1H), 3.47 (s, 1H), 3.33 (s, 1H), 2.63 (s, 3H), 1.58 (d, J = 7.5 Hz, 2H), 1.33 (d, J = 9.0 Hz, 4H), 0.90 (t, J = 6.8 Hz, 3H). |
| 419 | 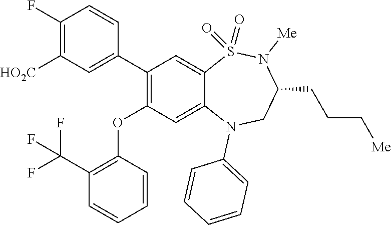 [SMILES: CCCC[C@@H]1CN(c2ccccc2)c2cc(Oc3ccccc3C(F)(F)F)c(-c3ccc(F)c(C(=O)O)c3)cc2S(=O)(=O)N1C] | [M + H]⁺ 643.2 | ¹H NMR (400 MHz, DMSO-d₆) δ 8.06 (d, J = 6.6 Hz, 1H), 7.92 (s, 1H), 7.86-7.78 (m, 1H), 7.70 (d, J = 7.6 Hz, 1H), 7.61 (t, J = 7.9 Hz, 1H), 7.39 (t, J = 10.1, 9.3 Hz, 1H), 7.32- 7.24 (m, 2H), 7.19 (t, J = 8.3, 7.7 Hz, 2H), 6.87 (d, J = 8.2 Hz, 2H), 6.81 (t, J = 7.3 Hz, 1H), 6.63 (s, 1H), 4.10 (d, J = 15.9 Hz, 1H), 3.77-3.67 (m, 1H), 3.56-3.45 (m, 1H), 2.65 (s, 3H), 1.62-1.52 (m, 2H), 1.41-1.28 (m, 4H), 0.90 (t, J = 6.9 Hz, 3H). |
| 420 | 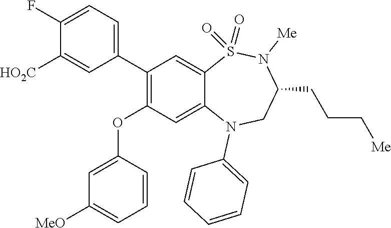 [SMILES: CCCC[C@@H]1CN(c2ccccc2)c2cc(Oc3cccc(OC)c3)c(-c3ccc(F)c(C(=O)O)c3)cc2S(=O)(=O)N1C] | [M + H]⁺ 605.3 | ¹H NMR (400 MHz, DMSO) δ 13.38 (s, 1H), 8.09-8.04 (m, 1H), 7.89- 7.84 (m, 1H), 7.42-7.35 (m, 1H), 7.24 (t, J = 8.2 Hz, 1H), 7.18 (t, J = 7.8 Hz, 2H), 6.88-6.76 (m, 4H), 6.75-6.60 (m, 4H), 4.12-4.03 (m, 1H), 3.79-3.71 (m, 1H), 3.70 (s, 3H), 3.50-3.39 (m, 1H), 2.61 (s, 3H), 1.59-1.55 (m, 2H), 1.38-1.30 (m, 4H), 0.90 (t, J = 6.8 Hz, 3H). |
| 421 | 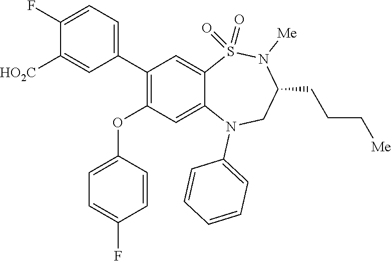 [SMILES: CCCC[C@@H]1CN(c2ccccc2)c2cc(Oc3ccc(F)cc3)c(-c3ccc(F)c(C(=O)O)c3)cc2S(=O)(=O)N1C] | [M + H]⁺ 593.4 | ¹H NMR (400 MHz, DMSO) δ 13.38 (s, 1H), 8.11-8.04 (m, 1H), 7.95- 7.88 (m, 1H), 7.86 (s, 1H), 7.41 (dd, J = 10.7, 8.6 Hz, 1H), 7.23-7.15 (m, 6H), 6.87-6.77 (m, 3H), 6.57 (s, 1H), 4.07 (d, J = 15.9 Hz, 1H), 3.77- 3.70 (m, 1H), 3.48-3.43 (m, 1H), 2.62 (s, 3H), 1.60-1.54 (m, 2H), 1.42-1.22 (m, 4H), 0.90 (t, J = 6.9 Hz, 3H). |
| 422 | 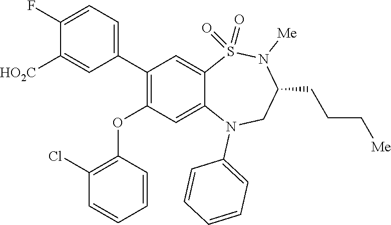 [SMILES: CCCC[C@@H]1CN(c2ccccc2)c2cc(Oc3ccccc3Cl)c(-c3ccc(F)c(C(=O)O)c3)cc2S(=O)(=O)N1C] | [M + H]⁺ 609.3 | ¹H NMR (400 MHz, DMSO) δ 13.38 (s, 1H), 8.18-8.12 (m, 1H), 7.96- 7.91 (m, 1H), 7.89 (s, 1H), 7.53 (dd, J = 8.0, 1.5 Hz, 1H), 7.43 (t, J = 10.1 Hz, 1H), 7.33 (ddd, J = 8.6, 7.1, 1.5 Hz, 1H), 7.32-7.25 (m, 1H), 7.24- 7.13 (m, 3H), 6.89-6.76 (m, 3H), 6.41 (s, 1H), 4.06 (d, J = 15.9 Hz, 1H), 3.76-3.69 (m, 1H), 3.48-3.44 (m, 1H), 2.62 (s, 3H), 1.58-1.54 (m, 2H), 1.41-1.28 (m, 4H), 0.90 (t, J = 6.8 Hz, 3H). |
| 423 | 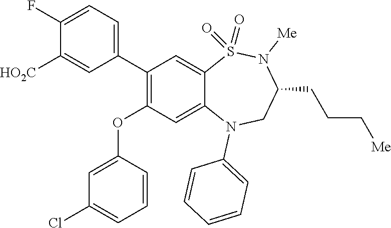 [SMILES: CCCC[C@@H]1CN(c2ccccc2)c2cc(Oc3cccc(Cl)c3)c(-c3ccc(F)c(C(=O)O)c3)cc2S(=O)(=O)N1C] | [M + H]⁺ 609.4 | ¹H NMR (400 MHz, DMSO) δ 13.37 (s, 1H), 8.05 (dd, J = 7.0, 2.5 Hz, 1H), 7.88 (s, 1H), 7.92-7.83 (m, 1H), 7.40 (dd, J = 10.6, 8.6 Hz, 1H), 7.35 (t, J = 8.1 Hz, 1H), 7.26 (t, J = 2.2 Hz, 1H), 7.24-7.14 (m, 3H), 7.07 (dd, J = 8.2, 2.4 Hz, 1H), 6.89 (d, J = 8.2 Hz, 2H), 6.82 (t, J = 7.3 Hz, 1H), 6.70 (s, 1H), 4.10 (d, J = 16.0 Hz, 1H), 3.76-3.70 (m, 1H), 3.51-3.47 (m, 1H), 2.64 (s, 3H), 1.62-1.53 (m, 2H), 1.42-1.27 (m, 4H), 0.90 (t, J = 6.9 Hz, 3H). |
| 424 | 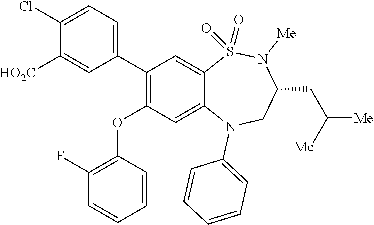 [SMILES: CC(C)C[C@@H]1CN(c2ccccc2)c2cc(Oc3ccccc3F)c(-c3ccc(Cl)c(C(=O)O)c3)cc2S(=O)(=O)N1C] | [M + H]⁺ 609.1 | ¹H NMR (400 MHz, DMSO-d₆) δ 13.45 (s, 1H), 7.96 (s, 1H), 7.83 (s, 1H), 7.74 (dd, J = 8.4, 2.3 Hz, 1H), 7.59 (d, J = 8.4 Hz, 1H), 7.30-7.21 (m, 2H), 7.12 (tdd, J = 8.5, 5.7, 3.4 Hz, 4H), 6.73 (dd, J = 8.1, 5.7 Hz, 3H), 6.46 (s, 1H), 3.95 (d, J = 16.1 Hz, 1H), 3.84-3.70 (m, 1H), 3.41- 3.31 (m, 1H), 2.53 (s, 3H), 1.66- 1.55 (m, 1H), 1.53-1.42 (m, 1H), 1.31-1.21 (m, 1H), 0.85 (d, J = 6.6 Hz, 3H), 0.81 (d, J = 6.5 Hz, 3H). |
| 425 | 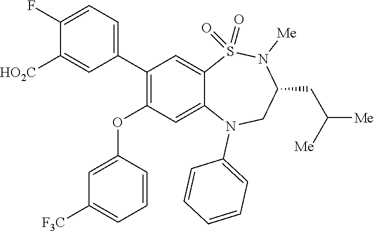 [SMILES: CC(C)C[C@@H]1CN(c2ccccc2)c2cc(Oc3cccc(C(F)(F)F)c3)c(-c3ccc(F)c(C(=O)O)c3)cc2S(=O)(=O)N1C] | [M + H]⁺ 643.4 | ¹H NMR (400 MHz, DMSO) δ 13.37 (s, 1H), 8.09-8.04 (m, 1H), 7.94- 7.85 (m, 2H), 7.60-7.51 (m, 2H), 7.47 (d, J = 7.7 Hz, 1H), 7.40 (m, 2H), 7.17 (dd, J = 8.6, 7.2 Hz, 2H), 6.86 (d, J = 8.1 Hz, 2H), 6.80 (t, J = 7.3 Hz, 1H), 6.74 (s, 1H), 4.05 (d, J = 16.1 Hz, 1H), 3.90-3.82 (m, 1H), 3.48-3.36 (m, 1H), 2.62 (s, 3H), 1.75-1.67 (m, 1H), 1.60-1.52 (m, 1H), 1.39-1.33 (m, 1H), 0.91 (dd, J = 14.1, 6.6 Hz, 6H). |
| 426 | 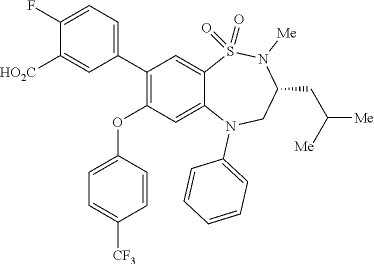 [SMILES: CC(C)C[C@@H]1CN(c2ccccc2)c2cc(Oc3ccc(C(F)(F)F)cc3)c(-c3ccc(F)c(C(=O)O)c3)cc2S(=O)(=O)N1C] | [M + H]⁺ 643.2 | ¹H NMR (400 MHz, DMSO) δ 13.38 (s, 1H), 8.04 (dd, J = 7.0, 2.5 Hz, 1H), 7.93 (s, 1H), 7.88-7.82 (m, 1H), 7.68 (d, J = 8.6 Hz, 2H), 7.40 (dd, J = 10.7, 8.6 Hz, 1H), 7.26- 7.16 (m, 4H), 6.90-6.78 (m, 4H), 4.07 (d, J = 16.3 Hz, 1H), 3.88 (m, 1H), 3.45-3.43 (m, 1H), 2.63 (s, 3H), 1.77-1.66 (m, 1H), 1.62-1.51 (m, 1H), 1.41-1.32 (m, 0H), 0.92 (dd, J = 14.1, 6.5 Hz, 6H). |
| 427 | 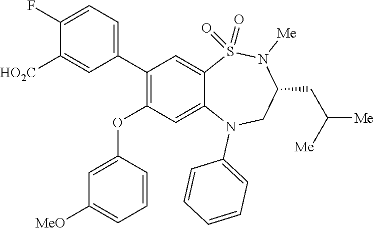 [SMILES: COc1cccc(Oc2cc3c(cc2-c2ccc(F)c(C(=O)O)c2)S(=O)(=O)N(C)[C@H](CC(C)C)CN3c2ccccc2)c1] | [M + H]⁺ 605.5 | ¹H NMR (400 MHz, DMSO) δ 13.38 (s, 1H), 8.13-8.06 (m, 1H), 7.96- 7.88 (m, 1H), 7.88 (s, 1H), 7.43 (dd, J = 10.7, 8.6 Hz, 1H), 7.24 (t, J = 8.2 Hz, 1H), 7.17 (t, J = 7.2 Hz, 2H), 6.85-6.75 (m, 3H), 6.77-6.63 (m, 4H), 4.03 (d, J = 16.4 Hz, 1H), 3.93- 3.83 (m, 1H), 3.70 (s, 3H), 3.38- 3.35 (m, 1H), 2.59 (s, 3H), 1.75- 1.66 (m, 1H), 1.59-1.50 (m, 1H), 1.41-1.29 (m, 1H), 0.91 (dd, J = 14.9, 6.5 Hz, 6H). |
| 428 | 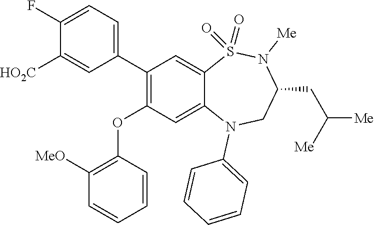 [SMILES: COc1ccccc1Oc1cc2c(cc1-c1ccc(F)c(C(=O)O)c1)S(=O)(=O)N(C)[C@H](CC(C)C)CN2c1ccccc1] | [M + H]⁺ 605.3 | |
| 429 | 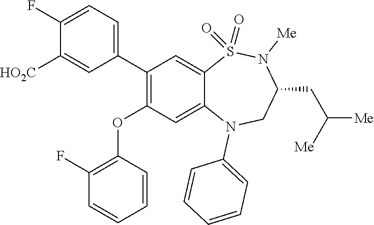 [SMILES: CC(C)C[C@@H]1CN(c2ccccc2)c2cc(Oc3ccccc3F)c(-c3ccc(F)c(C(=O)O)c3)cc2S(=O)(=O)N1C] | [M + H]⁺ 593.3 | |
| 430 | 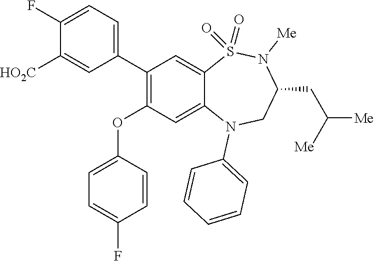 [SMILES: CC(C)C[C@@H]1CN(c2ccccc2)c2cc(Oc3ccc(F)cc3)c(-c3ccc(F)c(C(=O)O)c3)cc2S(=O)(=O)N1C] | [M + H]⁺ 593.4 | |
| 431 | 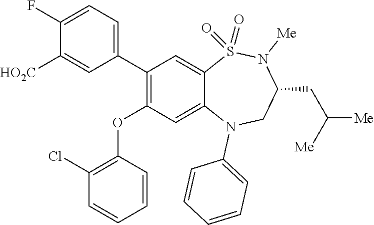 [SMILES: CC(C)C[C@@H]1CN(c2ccccc2)c2cc(Oc3ccccc3Cl)c(-c3ccc(F)c(C(=O)O)c3)cc2S(=O)(=O)N1C] | [M + H]⁺ 609.3 | |
| 432 | 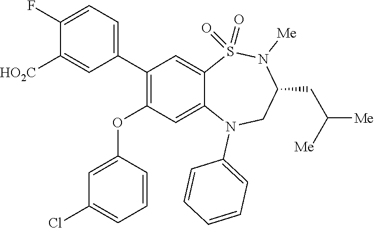 [SMILES: CC(C)C[C@@H]1CN(c2ccccc2)c2cc(Oc3cccc(Cl)c3)c(-c3ccc(F)c(C(=O)O)c3)cc2S(=O)(=O)N1C] | [M + H]⁺ 609.5 | |
| 433 | 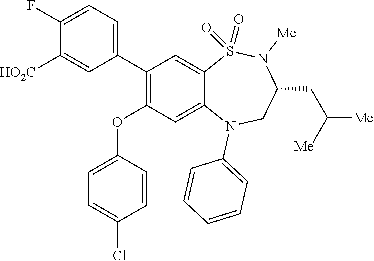 [SMILES: CC(C)C[C@@H]1CN(c2ccccc2)c2cc(Oc3ccc(Cl)cc3)c(-c3ccc(F)c(C(=O)O)c3)cc2S(=O)(=O)N1C] | [M + H]⁺ 609.6 | |
| 434 | 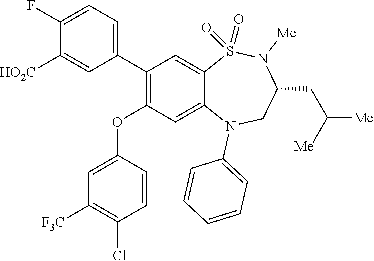 [SMILES: CC(C)C[C@@H]1CN(c2ccccc2)c2cc(Oc3ccc(Cl)c(C(F)(F)F)c3)c(-c3ccc(F)c(C(=O)O)c3)cc2S(=O)(=O)N1C] | [M + H]⁺ 677.2 | |
| 435 | 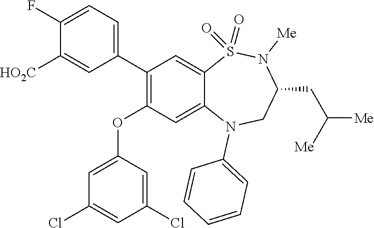 [SMILES: CC(C)C[C@@H]1CN(c2ccccc2)c2cc(Oc3cc(Cl)cc(Cl)c3)c(-c3ccc(F)c(C(=O)O)c3)cc2S(=O)(=O)N1C] | [M + H]⁺ 643.3 | |
| 436 | 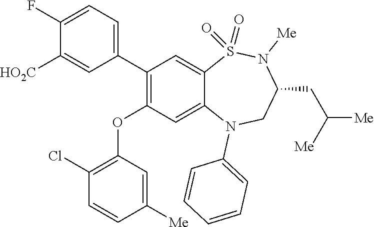 [SMILES: Cc1ccc(Cl)c(Oc2cc3c(cc2-c2ccc(F)c(C(=O)O)c2)S(=O)(=O)N(C)[C@H](CC(C)C)CN3c2ccccc2)c1] | [M + H]⁺ 623.3 | |
| 437 | 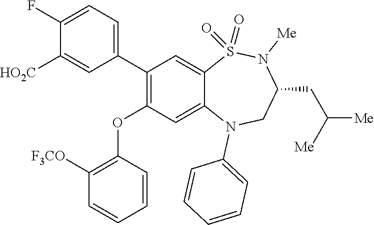 [SMILES: CC(C)C[C@@H]1CN(c2ccccc2)c2cc(Oc3ccccc3OC(F)(F)F)c(-c3ccc(F)c(C(=O)O)c3)cc2S(=O)(=O)N1C] | [M + H]⁺ 659.2 | |
| 438 | 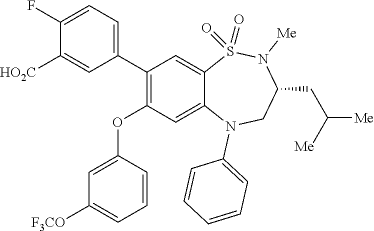 [SMILES: CC(C)C[C@@H]1CN(c2ccccc2)c2cc(Oc3cccc(OC(F)(F)F)c3)c(-c3ccc(F)c(C(=O)O)c3)cc2S(=O)(=O)N1C] | [M + H]⁺ 659.2 | |
| 439 | 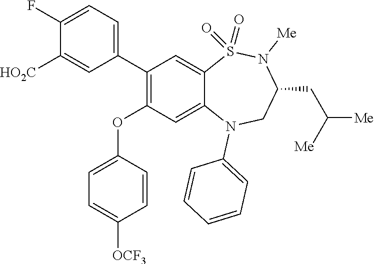 [SMILES: CC(C)C[C@@H]1CN(c2ccccc2)c2cc(Oc3ccc(OC(F)(F)F)cc3)c(-c3ccc(F)c(C(=O)O)c3)cc2S(=O)(=O)N1C] | [M + H]⁺ 659.4 | |
| 440 | 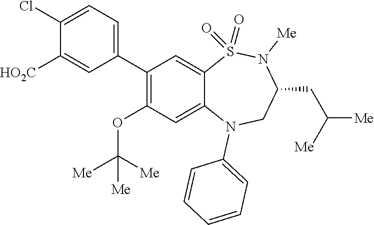 [SMILES: CC(C)C[C@@H]1CN(c2ccccc2)c2cc(OC(C)(C)C)c(-c3ccc(Cl)c(C(=O)O)c3)cc2S(=O)(=O)N1C] | [M + H]⁺ 571.3 | ¹H NMR (400 MHz, DMSO-d₆) δ 13.39 (s, 1H), 7.90 (s, 1H), 7.72 (s, 1H), 7.63 (d, J = 8.5 Hz, 1H), 7.54 (d, J = 8.3 Hz, 1H), 7.18 (t, J = 7.9 Hz, 2H), 6.88 (s, 1H), 6.81-6.74 (m, 3H), 3.99 (d, J = 16.2 Hz, 1H), 3.87- 3.82 (m, 1H), 3.45-3.22 (m, 1H), 2.49 (s, 3H), 1.66-1.61 (m, 1H), 1.50-1.46 (m, 1H), 1.35-1.24 (m, 1H), 1.10 (s, 9H), 0.87 (d, J = 6.6 Hz, 3H), 0.83 (d, J = 6.5 Hz, 3H). |
| 441 | 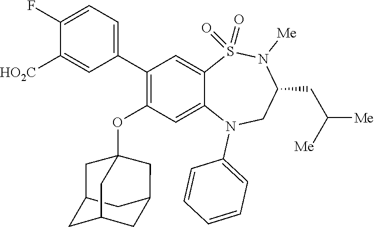 [SMILES: CC(C)C[C@@H]1CN(c2ccccc2)c2cc(OC34CC5CC(CC(C5)C3)C4)c(-c3ccc(F)c(C(=O)O)c3)cc2S(=O)(=O)N1C] | [M + H]⁺ 633.2 | |
| 442 | 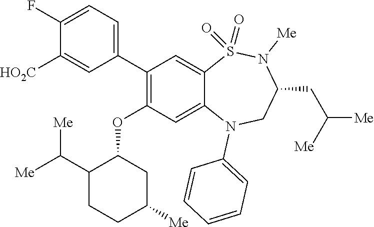 [SMILES: CC(C)C[C@@H]1CN(c2ccccc2)c2cc(O[C@@H]3C[C@H](C)CCC3C(C)C)c(-c3ccc(F)c(C(=O)O)c3)cc2S(=O)(=O)N1C] | [M + H]⁺ 637.6 | |
| 443 | 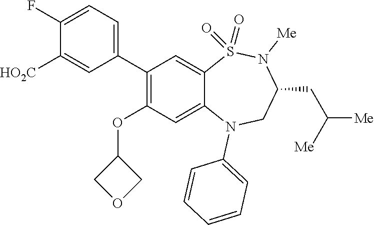 [SMILES: CC(C)C[C@@H]1CN(c2ccccc2)c2cc(OC3COC3)c(-c3ccc(F)c(C(=O)O)c3)cc2S(=O)(=O)N1C] | [M + H]⁺ 555.2 | |
| 444 | 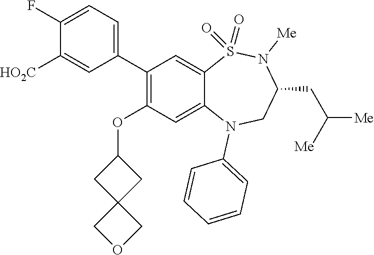 [SMILES: CC(C)C[C@@H]1CN(c2ccccc2)c2cc(OC3CC4(COC4)C3)c(-c3ccc(F)c(C(=O)O)c3)cc2S(=O)(=O)N1C] | [M + H]⁺ 595.2 | |
| 445 | 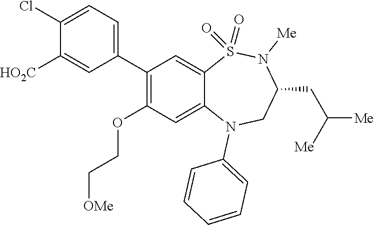 [SMILES: COCCOc1cc2c(cc1-c1ccc(Cl)c(C(=O)O)c1)S(=O)(=O)N(C)[C@H](CC(C)C)CN2c1ccccc1] | [M + H]⁺ 573.4 | ¹H NMR (400 MHz, DMSO) δ 13.46 (s, 1H), 8.04 (t, J = 1.9 Hz, 1H), 7.77 (d, J = 10.0 Hz, 2H), 7.62 (dd, J = 8.4, 1.2 Hz, 1H), 7.22 (t, J = 7.8 Hz, 2H), 7.08 (s, 1H), 6.80 (dd, J = 8.2, 5.4 Hz, 3H), 4.21-4.03 (m, 3H), 3.95 (s, 1H), 3.58 (t, J = 4.4 Hz, 2H), 3.36-3.35 (m, 1H), 3.23 (s, 3H), 2.53 (s, 3H), 1.71 (s, 1H), 1.54 (s, 1H), 1.37 (ddd, J = 14.5, 9.4, 5.4 Hz, 1H), 0.92 (dd, J = 15.8, 6.5 Hz, 6H). |
| 446 | 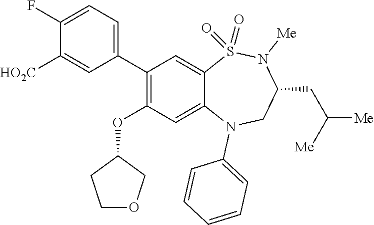 [SMILES: CC(C)C[C@@H]1CN(c2ccccc2)c2cc(O[C@H]3CCOC3)c(-c3ccc(F)c(C(=O)O)c3)cc2S(=O)(=O)N1C] | [M + H]⁺ 569.4 | |
| 447 | 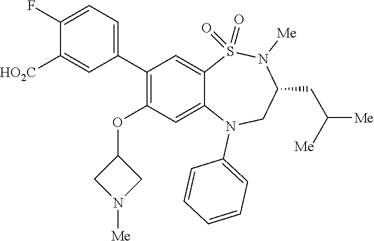 [SMILES: CC(C)C[C@@H]1CN(c2ccccc2)c2cc(OC3CN(C)C3)c(-c3ccc(F)c(C(=O)O)c3)cc2S(=O)(=O)N1C] | [M + H]⁺ 568. | |
| 448 | 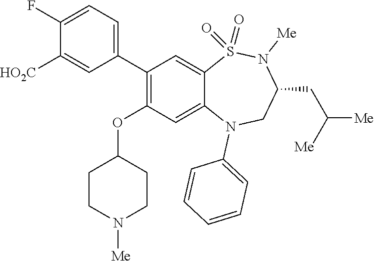 [SMILES: CC(C)C[C@@H]1CN(c2ccccc2)c2cc(OC3CCN(C)CC3)c(-c3ccc(F)c(C(=O)O)c3)cc2S(=O)(=O)N1C] | [M + H]⁺ 596.5 | |
| 449 | 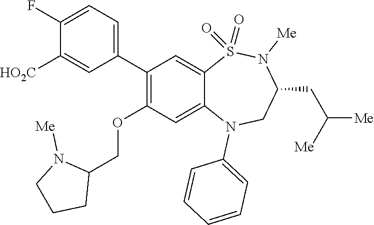 [SMILES: CC(C)C[C@@H]1CN(c2ccccc2)c2cc(OCC3CCCN3C)c(-c3ccc(F)c(C(=O)O)c3)cc2S(=O)(=O)N1C] | [M + H]⁺ 596.5 | |
| 450 | 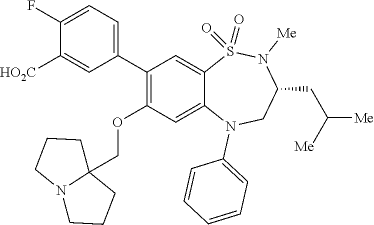 [SMILES: CC(C)C[C@@H]1CN(c2ccccc2)c2cc(OCC34CCCN3CCC4)c(-c3ccc(F)c(C(=O)O)c3)cc2S(=O)(=O)N1C] | [M + H]⁺ 622.6 | |
| 451 | 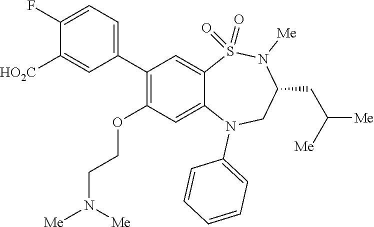 [SMILES: CC(C)C[C@@H]1CN(c2ccccc2)c2cc(OCCN(C)C)c(-c3ccc(F)c(C(=O)O)c3)cc2S(=O)(=O)N1C] | [M + H]⁺ 570.5 | |
| 452 | 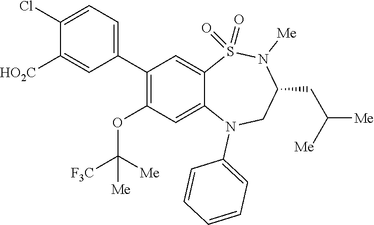 [SMILES: CC(C)C[C@@H]1CN(c2ccccc2)c2cc(OC(C)(C)C(F)(F)F)c(-c3ccc(Cl)c(C(=O)O)c3)cc2S(=O)(=O)N1C] | [M + H]⁺ 625.2 | ¹H NMR (400 MHz, DMSO) δ 13.50 (s, 1H), 7.93 (d, J = 2.2 Hz, 1H), 7.83 (s, 1H), 7.70-7.61 (m, 2H), 7.25 (t, J = 7.9 Hz, 2H), 7.05 (s, 1H), 6.88- 6.82 (m, 3H), 4.09 (d, J = 16.1 Hz, 1H), 3.96-3.87 (m, 1H), 3.44 (s, 1H), 2.57 (s, 3H), 1.75-1.66 (m, 1H), 1.59-1.50 (m, 1H), 1.37 (m, 1H), 1.22 (s, 3H), 1.16 (s, 3H), 0.92 (dd, J = 13.7, 6.5 Hz, 6H). |
| 453 | 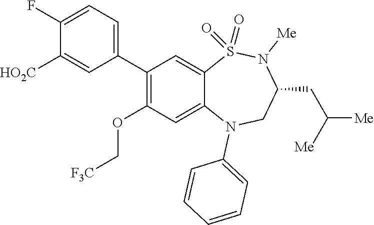 [SMILES: CC(C)C[C@@H]1CN(c2ccccc2)c2cc(OCC(F)(F)F)c(-c3ccc(F)c(C(=O)O)c3)cc2S(=O)(=O)N1C] | [M + H]⁺ 581.4 | |
| 454 | 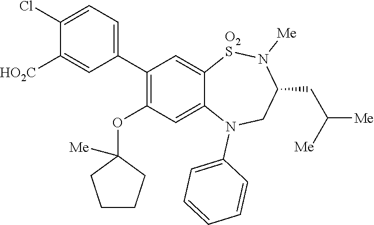 [SMILES: CC(C)C[C@@H]1CN(c2ccccc2)c2cc(OC3(C)CCCC3)c(-c3ccc(Cl)c(C(=O)O)c3)cc2S(=O)(=O)N1C] | [M + H]⁺ 597.3 | ¹H NMR (400 MHz, DMSO) δ 13.46 (s, 1H), 7.97 (d, J = 2.3 Hz, 1H), 7.75 (s, 1H), 7.69 (dd, J = 8.3, 2.3 Hz, 1H), 7.61 (d, J = 8.4 Hz, 1H), 7.26 (t, J = 7.9 Hz, 2H), 6.91-6.79 (m, 4H), 4.05 (d, J = 16.0 Hz, 1H), 3.97-3.84 (m, 1H), 3.52-3.38 (m, 1H), 2.57 (s, 3H), 2.01-1.84 (m, 2H), 1.76-1.64 (m, 1H), 1.59-1.46 (m, 7H), 1.39- 1.29 (m, 4H), 0.92 (dd, J = 13.3, 6.5 Hz, 6H). |
| 455 | 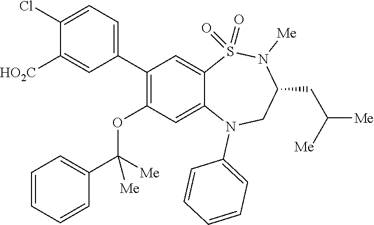 [SMILES: CC(C)C[C@@H]1CN(c2ccccc2)c2cc(OC(C)(C)c3ccccc3)c(-c3ccc(Cl)c(C(=O)O)c3)cc2S(=O)(=O)N1C] | [M + H]⁺ 633.4 | ¹H NMR (400 MHz, DMSO) δ 13.49 (s, 1H), 8.09 (s, 1H), 7.83 (dd, J = 8.4, 2.4 Hz, 1H), 7.73 (s, 1H), 7.65 (d, J = 8.3 Hz, 1H), 7.28-7.19 (m, 5H), 7.10 (dd, J = 8.6, 7.2 Hz, 2H), 6.79 (d, J = 7.3 Hz, 1H), 6.49 (d, J = 8.1 Hz, 2H), 6.16 (s, 1H), 3.89-3.75 (m, 2H), 3.27-3.21 (m, 1H), 2.53 (s, 3H), 1.71-1.61 (m, 4H), 1.58 (s, 3H), 1.54-1.41 (m, 1H), 1.32-1.21 (m, 1H), 0.88 (dd, J = 16.0, 6.5 Hz, 6H). |
| 456 | 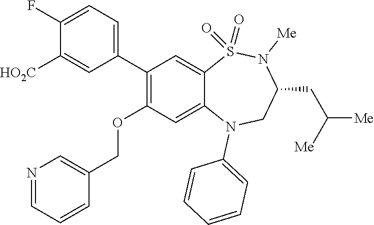 [SMILES: CC(C)C[C@@H]1CN(c2ccccc2)c2cc(OCc3cccnc3)c(-c3ccc(F)c(C(=O)O)c3)cc2S(=O)(=O)N1C] | [M + H]⁺ 590.2 | |
| 457 | 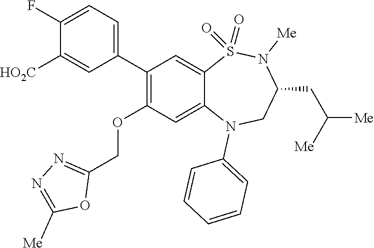 [SMILES: Cc1nnc(COc2cc3c(cc2-c2ccc(F)c(C(=O)O)c2)S(=O)(=O)N(C)[C@H](CC(C)C)CN3c2ccccc2)o1] | [M + H]⁺ 595.4 | |
| 458 | 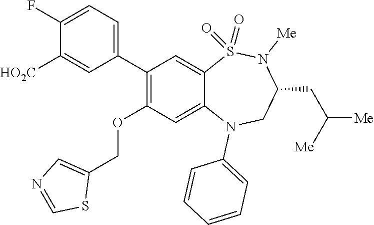 [SMILES: CC(C)C[C@@H]1CN(c2ccccc2)c2cc(OCc3cncs3)c(-c3ccc(F)c(C(=O)O)c3)cc2S(=O)(=O)N1C] | [M + H]⁺ 596.3 | |
| 459 | 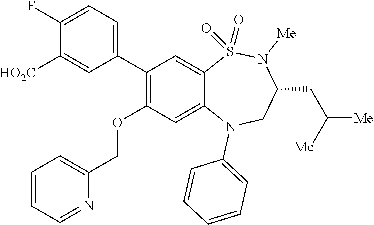 [SMILES: CC(C)C[C@@H]1CN(c2ccccc2)c2cc(OCc3ccccn3)c(-c3ccc(F)c(C(=O)O)c3)cc2S(=O)(=O)N1C] | [M + H]⁺ 590.5 | |
| 460 | 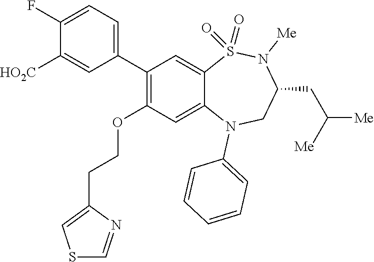 [SMILES: CC(C)C[C@@H]1CN(c2ccccc2)c2cc(OCCc3cscn3)c(-c3ccc(F)c(C(=O)O)c3)cc2S(=O)(=O)N1C] | [M + H]⁺ 610.5 | |
| 461 | 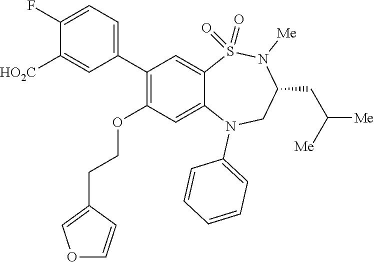 [SMILES: CC(C)C[C@@H]1CN(c2ccccc2)c2cc(OCCc3ccoc3)c(-c3ccc(F)c(C(=O)O)c3)cc2S(=O)(=O)N1C] | [M + H]⁺ 593.5 | |
| 462 | 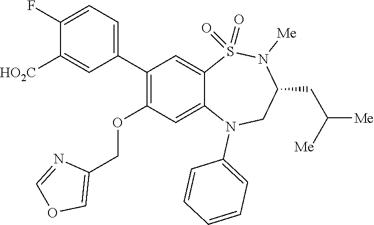 [SMILES: CC(C)C[C@@H]1CN(c2ccccc2)c2cc(OCc3cocn3)c(-c3ccc(F)c(C(=O)O)c3)cc2S(=O)(=O)N1C] | [M + H]⁺ 580.4 | |
| 463 | 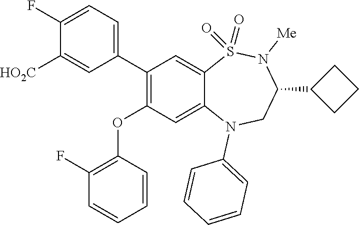 [SMILES: CN1[C@H](C2CCC2)CN(c2ccccc2)c2cc(Oc3ccccc3F)c(-c3ccc(F)c(C(=O)O)c3)cc2S1(=O)=O] | [M + H]⁺ 591.2 | |
| 464 | 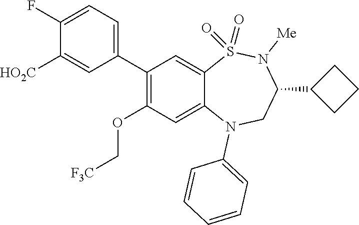 [SMILES: CN1[C@H](C2CCC2)CN(c2ccccc2)c2cc(OCC(F)(F)F)c(-c3ccc(F)c(C(=O)O)c3)cc2S1(=O)=O] | [M + H]⁺ 579.5 | |
| 465 | 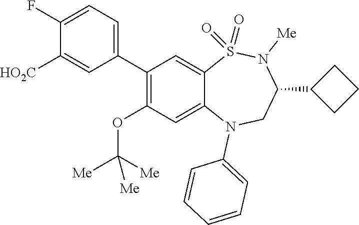 [SMILES: CN1[C@H](C2CCC2)CN(c2ccccc2)c2cc(OC(C)(C)C)c(-c3ccc(F)c(C(=O)O)c3)cc2S1(=O)=O] | [M + H]⁺ 553.4 | |

### Ex.466: Synthesis of (R)-5-(5-(3,3-difluorocyclobutyl)-7-(2-flurophenoxy)-3-isobutyl-2-methl-1,1-dioxido-2,3,4,5-tetrahydrobenzo[f][1,2,5]thiadiazepin-8-yl)-2-fluorobenzoic acid

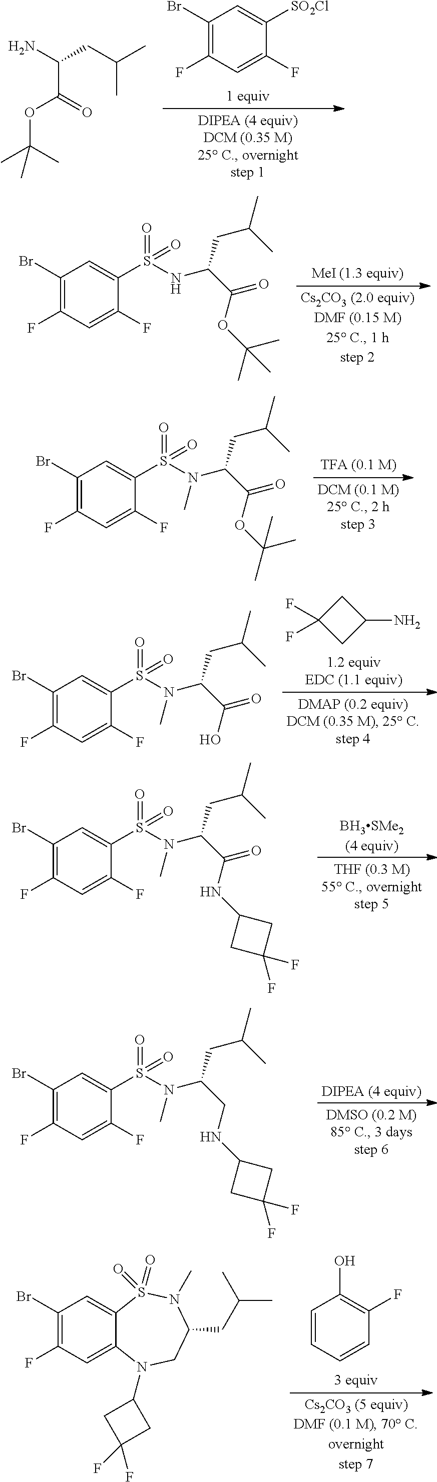

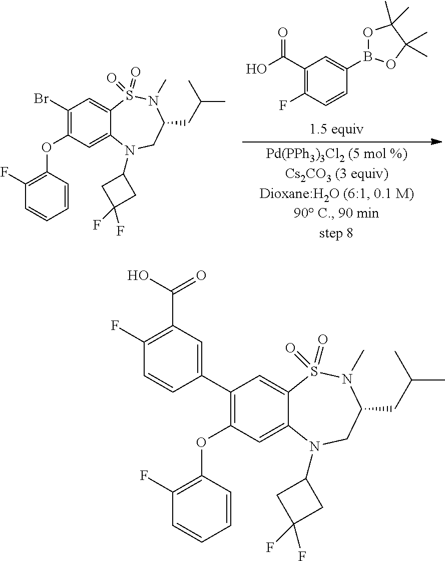 [SMILES: CC(C)C[C@@H]1CN(C2CC(F)(F)C2)c2cc(Oc3ccccc3F)c(Br)cc2S(=O)(=O)N1C ; CC1(C)OB(c2ccc(F)c(C(=O)O)c2)OC1(C)C ; CC(C)C[C@@H]1CN(C2CC(F)(F)C2)c2cc(Oc3ccccc3F)c(-c3ccc(F)c(C(=O)O)c3)cc2S(=O)(=O)N1C]

Step 1

In a 250 mL round bottom flask equipped with a stir bar, tert-butyl D-leucinate HCl (2.5 g, 1.0 equiv., CAS #: 13081-32-8) and 5-bromo-2,4-difluorobenzenesulfonyl chloride (3.26 g, 11.17 mmol, CAS#287172-61-6) were dissolved in dry DCM (31.9 mL) under nitrogen at room temperature. Subsequently N-ethyl-N-isopropylpropan-2-amine (7.79 mL, 4.0 equiv.) was added dropwise, and reaction mixture was stirred for 6 h at room temperature. Reaction progress was monitored using LC-MS. Once completed, the reaction mixture was diluted with additional DCM and washed with water (xl) and brine (xl). The organic layer was dried over sodium sulfate and concentrated under vacuum to afford tert-butyl((5-bromo-2,4-difluorophenyl)sulfonyl)-D-leucinate. ESI MS m/z=440.0 [M−H]⁻. Crude was transferred to the next reaction without any further purification.

Step 2

In a 250 mL round bottom flask equipped with a stir bar, tert-butyl ((5-bromo-2,4-difluorophenyl)sulfonyl)-D-leucinate (4.94 g, 1.0 equiv.) was dissolved in dry DMF (74.5 mL), followed by addition of cesium carbonate (7.28 g, 2.0 equiv.). Subsequently, methyl iodide (0.908 mL, 1.3 equiv.) was added dropwise, and reaction mixture was stirred for 1 h at room temperature. Reaction progress was monitored using LC-MS. Once completed, the reaction mixture was diluted with MTBE and extracted with water. The aqueous layer was further washed an additional three times with MTBE. Then, combined organic layers were washed with water and brine and dried over sodium sulfate and concentrated under vacuum to afford tert-butyl N-((5-bromo-2,4-difluorophenyl)sulfonyl)-N-methyl-D-leucinate. ESI MS m/z=354.0 [M-Boc]. Crude was transferred to the next reaction without any further purification.

Step 3

In a 250 mL round bottom flask equipped with a stir bar, tert-butyl N-((5-bromo-2,4-difluorophenyl)sulfonyl)-N-methyl-D-leucinate (5.10 g, 11.17 mmol) was dissolved in dry DCM (55.9 mL, 0.1M) at room temperature, followed by addition of trifluoroacetic acid (55.9 mL) dropwise and reaction mixture was stirred for 2 h. Mixture was concentrated under vacuum to afford N-((5-bromo-2,4-difluorophenyl)sulfonyl)-N-methyl-D-leucine. ESI MS m/z=400.0 [M−H]. Crude was transferred to the next reaction without any further purification.

Step 4

In a 20 mL vial equipped with a stir bar, 3,3-difluorocyclobutan-1-amine HCl (215 mg, 1.2 equiv., CAS #637031-93-7), N-((5-bromo-2,4-difluorophenyl)sulfonyl)-N-methyl-D-leucine (500 mg, 1.0 equiv.), 3-(((ethylimino)methylene)amino)-N,N-dimethylpropan-1-amine hydrochloride (263 mg, 1.1 equiv., CAS #25952-53-8) and N,N-dimethylpyridin-4-amine (30.5 mg, 0.2 equiv.) were taken under nitrogen atmosphere. Dry DCM (3.57 ml) was added subsequently, and reaction mixture was stirred overnight at room temperature. Mixture was concentrated under vacuum directly, and purified through normal phase silica column chromatography to afford (R)-2-((5-bromo-2,4-difluoro-N-methylphenyl)sulfonamido)-N-(3,3-difluorocyclobutyl)-4-methylpentanamide. ESI MS m/z=490.9 [M+H]⁺.

Step 5

In a 20 mL vial equipped with a stir bar, (R)-2-((5-bromo-2,4-difluoro-N-methylphenyl)sulfonamido)-N-(3,3-difluorocyclobutyl)-4-methylpentanamide (500 mg, 1.0 equiv.) was dissolved in dry tetrahydrofuran (3.4 mL). BH₃ DMS (2M in THF, 2.1 mL, 4.0 equiv. CAS #13292-87-0) was added dropwise at room temperature, and then reaction mixture was stirred at 55° C. overnight on a heating block. Reaction progress was monitored using LC-MS. Once completed, it was quenched with water, diluted with ethyl acetate. Layers were separated and the organic layer was further washed with water and brine. Crude was dried over sodium sulfate and concentrated under vacuum to afford (R)-5-bromo-N-(1-((3,3-difluorocyclobutyl)amino)-4-methylpentan-2-yl)-2,4-difluoro-N-methylbenzenesulfonamide. ESI MS m/z=475.0 [M+H]⁺. Crude was transferred to the next reaction without any further purification.

Step 6

In a 20 mL vial equipped with a stir bar, (R)-5-bromo-N-(1-((3,3-difluorocyclobutyl)amino)-4-methylpentan-2-yl)-2,4-difluoro-N-methylbenzenesulfonamide (0.486 g, 1.0 equiv.) was dissolved in DMSO (5.11 mL) at room temperature. DIPEA (0.714 ml, 4.0 equiv.) was added dropwise, and reaction mixture was stirred for 12 h at 70° C. on a heating block. Little conversation. Added 8 equiv (1.4 mL) DIPEA and stirred for 2 days at 85° C. Reaction progress was monitored LC-MS. Crude was directly transferred into a 100 g C18 gold column and purified through reversed phase flash chromatography to afford (R)-8-bromo-5-(3,3-difluorocyclobutyl)-7-fluoro-3-isobutyl-2-methyl-2,3,4,5-tetrahydrobenzo[f][1,2,5]thiadiazepine 1,1-dioxide (465 mg, 50% yield). ESI MS m/z=455.0 [M+H]⁺.

Step 7

In a 4 mL vial equipped with a stir bar, (R)-8-bromo-5-(3,3-difluorocyclobutyl)-7-fluoro-3-isobutyl-2-methyl-2,3,4,5-tetrahydrobenzo[f][1,2,5]thiadiazepine 1,1-dioxide (60 mg, 1.0 equiv.) was dissolved in DMF (1.318 ml). 2-Fluorophenol (44.3 mg, 3.0 equiv.) and cesium carbonate (215 mg, 5.0 equiv.) were added at room temperature, and then reaction mixture was stirred overnight at 70° C. on a heating block. Reaction mixture was directly loaded into a 30 g C18 gold column and purified through reversed phase flash chromatography to afford (R)-8-bromo-5-(3,3-difluorocyclobutyl)-7-(2-fluorophenoxy)-3-isobutyl-2-methyl-2,3,4,5-tetrahydrobenzo[f][1,2,5]thiadiazepine 1,1-dioxide (57 mg, 79% yield). ESI MS m/z=547.0 [M+H]⁺.

Step 8

In 4 mL reaction vial equipped with a stir bar, (R)-8-bromo-5-(3,3-difluorocyclobutyl)-7-(2-fluorophenoxy)-3-isobutyl-2-methyl-2,3,4,5-tetrahydrobenzo[f][1,2,5]thiadiazepine 1,1-dioxide (30 mg, 1.0 equiv.), 2-fluoro-5-(4,4,5,5-tetramethyl-1,3,2-dioxaborolan-2-yl)benzoic acid (21.9, 1.5 equiv.), bis(triphenylphosphine)palladium(II) chloride (3.85 mg, 0.1 equiv.) and cesium carbonate (53.6 mg, 3.0 equiv.) were combined neat under nitrogen atmosphere, followed by addition of dioxane (0.48 ml) and water (0.07 ml). Reaction mixture was stirred for 90 minutes at 90° C. on a heating block. Reaction mixture was quenched with formic acid (0.2 mL) and concentrated under vacuum. The crude residue was re-dissolved in 2.0 mL of dimethyl sulfoxide, passed through a 0.45 μm syringe filter, and purified by RPHPLC to afford (R)-5-(5-(3,3-difluorocyclobutyl)-7-(2-fluorophenoxy)-3-isobutyl-2-methyl-1,1-dioxido-2,3,4,5-tetrahydrobenzo[f][1,2,5]thiadiazepin-8-yl)-2-fluorobenzoic acid, Ex.466 (23 mg, 69% yield). ESI MS m/z=607.2 [M+H]⁺.

| Example | Structure | MS | NMR |
|---|---|---|---|
| 466 | 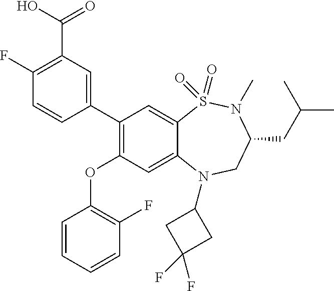 [SMILES: CC(C)C[C@@H]1CN(C2CC(F)(F)C2)c2cc(Oc3ccccc3F)c(-c3ccc(F)c(C(=O)O)c3)cc2S(=O)(=O)N1C] | [M + H]⁺ 607.2 | ¹H NMR (500 MHz, DMSO-d6) δ 13.37 (br, 1H), 8.02-7.95 (m, 1H), 7.83-7.74 (m, 2H), 7.47-7.19 (m, 5H), 6.27 (s, 1H), 3.97-3.67 (m, 2H), 3.16-2.80 (m, 3H), 2.56 (s, 3H), 2.25-2.07 (m, 1H), 1.76-1.60 (m, 1H), 1.58-1.41 (m, 1H), 1.29-1.19 (m, 1H), 0.94 (t, J = 6.0 Hz, 6H). |

The following compounds were prepared using a procedure analogous to that described for Ex.466:

| Example | Structure | MS |
|---|---|---|
| 467 | 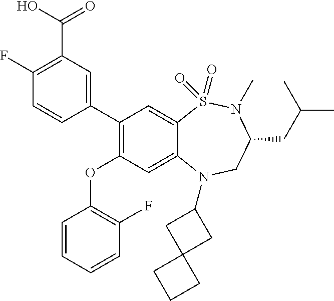 [SMILES: CC(C)C[C@@H]1CN(C2CC3(CCC3)C2)c2cc(Oc3ccccc3F)c(-c3ccc(F)c(C(=O)O)c3)cc2S(=O)(=O)N1C] | [M + H]⁺ 611.3 |
| 468 | 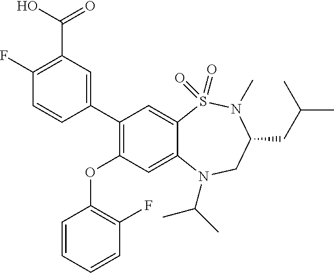 [SMILES: CC(C)C[C@@H]1CN(C(C)C)c2cc(Oc3ccccc3F)c(-c3ccc(F)c(C(=O)O)c3)cc2S(=O)(=O)N1C] | [M + H]⁺ 559.2 |
| 469 | 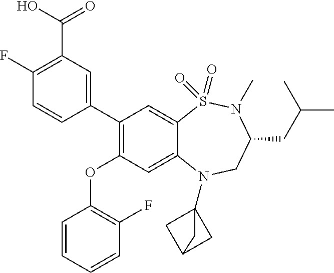 [SMILES: CC(C)C[C@@H]1CN(C23CC(C2)C3)c2cc(Oc3ccccc3F)c(-c3ccc(F)c(C(=O)O)c3)cc2S(=O)(=O)N1C] | [M + H]⁺ 583.2 |
| 470 | 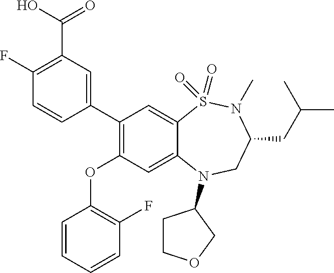 [SMILES: CC(C)C[C@@H]1CN([C@@H]2CCOC2)c2cc(Oc3ccccc3F)c(-c3ccc(F)c(C(=O)O)c3)cc2S(=O)(=O)N1C] | [M + H]⁺ 587.2 |
| 471 | 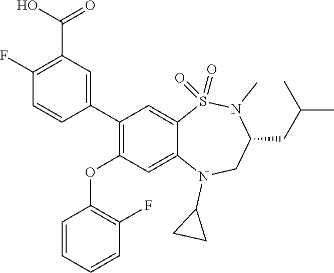 [SMILES: CC(C)C[C@@H]1CN(C2CC2)c2cc(Oc3ccccc3F)c(-c3ccc(F)c(C(=O)O)c3)cc2S(=O)(=O)N1C] | [M + H]⁺ 557.2 |
| 472 | 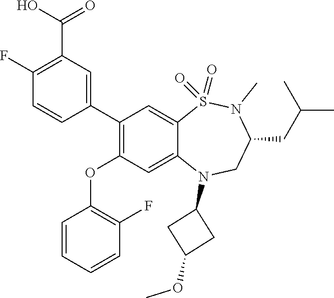 [SMILES: CO[C@H]1C[C@H](N2C[C@@H](CC(C)C)N(C)S(=O)(=O)c3cc(-c4ccc(F)c(C(=O)O)c4)c(Oc4ccccc4F)cc32)C1] | [M + H]⁺ 601.2 |
| 473 | 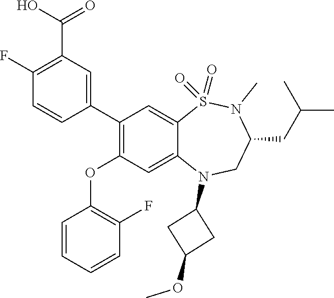 [SMILES: CO[C@H]1C[C@@H](N2C[C@@H](CC(C)C)N(C)S(=O)(=O)c3cc(-c4ccc(F)c(C(=O)O)c4)c(Oc4ccccc4F)cc32)C1] | [M + H]⁺ 601.2 |
| 474 | 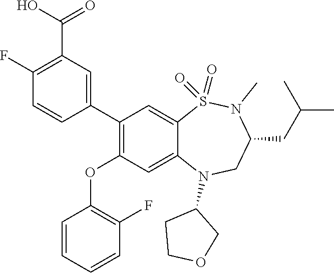 [SMILES: CC(C)C[C@@H]1CN([C@H]2CCOC2)c2cc(Oc3ccccc3F)c(-c3ccc(F)c(C(=O)O)c3)cc2S(=O)(=O)N1C] | [M + H]⁺ 587.2 |

### Ex.475: Synthesis of(R)-2-fluoro-5-(7-(4-fluorophenoxy)-3,5-diisobutyl-2-methyl-1,1-dioxido-2,3,4,5-tetrahydrobenzo[f][1,2,5]thiadiazepin-8-yl)benzoic acid

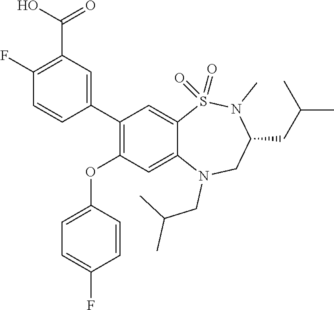 [SMILES: CC(C)C[C@@H]1CN(CC(C)C)c2cc(Oc3ccc(F)cc3)c(-c3ccc(F)c(C(=O)O)c3)cc2S(=O)(=O)N1C]

Step 1

A 1000-mL round bottom flask was equipped with a magnetic stir bar, cooled in an ice bath and charged with (R)-2-amino-4-methylpentanamide hydrochloride (15 g, 90 mmol), DCM (257 ml), N,N-diisopropylethylamine (39.3 ml, 225 mmol) and 5-bromo-2,4-difluorobenzenesulfonyl chloride (26.2 g, 90 mmol). The resulting mixture was stirred 15 h, then diluted with DCM and water. The biphasic mixture was shaken, the layers separated, and the organics shaken with 1 N hydrochloric acid (250 mL).

The phases were once again separated, and the organics were rinsed with 0.5 N HCl (500 mL), water (500 mL), and sat. aq. NaHCO₃, (250 mL). The combined organics were rinsed with brine (500 mL), dried over sodium sulfate, filtered and the filter cake rinsed with additional DCM. The combined filtrates were concentrated under reduced pressure to reveal (R)-2-((5-bromo-2,4-difluorophenyl)sulfonamido)-4-methylpentanamide (23.8 g, 61.8 mmol, 68.6% yield) as a white solid. ESI-MS m/z=385.0.

Step 2

To a 3000-mL round bottom flask containing (R)-2-((5-bromo-2,4-difluorophenyl)sulfonamido)-4-methylpentanamide (23.7 g, 61.5 mmol) were added acetonitrile (410 mL) and cesium carbonate (40.1 g, 123 mmol). The resulting thick slurry was stirred as iodomethane (3.85 ml, 61.5 mmol) was then added dropwise; the resulting mixture was stirred an additional 5 h, then filtered. The filter cake was rinsed with several additional portions of acetonitrile (3×15 mL) and the combined filtrates concentrated into a 2000-mL round bottom flask, affording an off-white gum. The flask was charged with a magnetic stir bar and THE (308 ml), giving a white suspension; the flask was then flushed with nitrogen, sealed, charged with BH₃-DMS (17.52 ml, 185 mmol) and heated to 50° C. After stirring overnight, the reaction mixture was brought to room temperature and quenched by slow, portionwise addition of methanol, then concentrated under reduced pressure to reveal the crude amine as a colorless gum. The crude material was dissolved in 8 N HCl, rinsed several times with DCM, then concentrated under reduced pressure to reveal the hydrochloride, (R)-N-(1-amino-4-methylpentan-2-yl)-5-bromo-2,4-difluoro-N-methylbenzenesulfonamide hydrochloride, as a colorless solid; a portion of this material was used without further purification as follows:

Step 3

To a 20-mL glass vial containing a magnetic stir bar and a pale yellow solution of (R)-N-(1-amino-4-methylpentan-2-yl)-5-bromo-2,4-difluoro-N-methylbenzenesulfonamide hydrochloride (0.150 g, 0.356 mmol) in DCE (2.5 mL) was added acetic acid (0.041 ml, 0.711 mmol), isobutyraldehyde (0.039 ml, 0.427 mmol) and finally sodium triacetoxyborohydride (0.188 g, 0.889 mmol). The resulting colorless mixture was stirred 4 h, then quenched with 1 N HCl, diluted with water, and phase-separated. The aqueous phase was extracted three times with EtOAc, and the combined organics concentrated under reduced pressure to reveal (R)-5-bromo-2,4-difluoro-N-(1-(isobutylamino)-4-methylpentan-2-yl)-N-methylbenzenesulfonamide (0.091 g, 58.0%) as a colorless solid.

Step 4

To a 20-mL glass vial containing a magnetic stir bar and (R)-5-bromo-2,4-difluoro-N-(1-(isobutylamino)-4-methylpentan-2-yl)-N-methylbenzenesulfonamide (0.091 g, 0.206 mmol) were added cesium carbonate (135 mg, 0.414 mmol) and DMF (1.5 mL). The vial was then heated to 95° C. and stirred overnight. The reaction mixture was allowed to cool, then partitioned between ethyl acetate and water. The aqueous phase was extracted with ethyl acetate and the combined organics concentrated under reduced pressure to reveal the crude cyclized amine. To this residue were added cesium carbonate (315 mg, 0.967 mmol), 4-fluorophenol (85 mg, 0.758 mmol), and DMSO (0.665 ml). The reaction mixture was heated to 65° C. and stirred overnight, then cooled and partitioned between ethyl acetate and water. The organics were rinsed with water (5×), dried over sodium sulfate, filtered and concentrated under reduced pressure to afford a crude residue which was charged to a 2-dram vial equipped with a magnetic stir bar. 2-fluoro-5-(4,4,5,5-tetramethyl-1,3,2-dioxaborolan-2-yl)benzoic acid (0.032 g, 0.119 mmol), cesium carbonate (0.093 g, 0.285 mmol), dioxane (0.8 ml), and water (0.2 ml) were added, and the resulting mixture sparged with nitrogen. PdCl₂(dppf) (10.43 mg, 0.014 mmol) was added and the reaction mixture heated to 65° C. After stirring overnight, the reaction mixture was concentrated under reduced pressure, then purified by RP-HPLC to afford (R)-2-fluoro-5-(7-(4-fluorophenoxy)-3,5-diisobutyl-2-methyl-1,1-dioxido-2,3,4,5-tetrahydrobenzo[f][1,2,5]thiadiazepin-8-yl)benzoic acid, Ex.475 (10 mg, 19%) as a colorless solid:

| Ex- | | |
|---|---|---|
| am- | | |
| ple | Structure | MS |
| 475 | 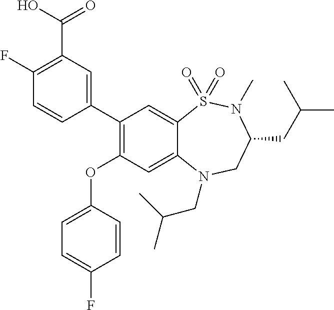 [SMILES: CC(C)C[C@@H]1CN(CC(C)C)c2cc(Oc3ccc(F)cc3)c(-c3ccc(F)c(C(=O)O)c3)cc2S(=O)(=O)N1C] | [M + H]⁺ 573.3 |

The following compound was prepared using a procedure analogous to that used for Ex.401 starting from (R)-2-chloro-5-(7-chloro-3-cyclohexyl-2,5-dimethyl-1,1-dioxido-2,3,4,5-tetrahydrobenzo[f][1,2,5]thiadiazepin-8-yl)benzoic acid:

| Example | Structure | MS | NMR |
|---|---|---|---|
| 476 | 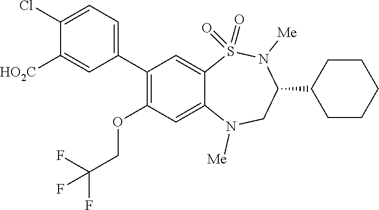 [SMILES: CN1C[C@@H](C2CCCCC2)N(C)S(=O)(=O)c2cc(-c3ccc(Cl)c(C(=O)O)c3)c(OCC(F)(F)F)cc21] | [M + H]⁺ 545.3 | ¹H NMR (400 MHz, DMSO) δ 13.30 (s, 1H), 7.90 (d, J = 6.9 Hz, 1H), 7.67 (m, 1H), 7.52 (s, 1H), 7.33 (t, J = 9.7 Hz, 1H), 6.62 (s, 1H), 4.93 (q, J = 8.8 Hz, 2H), 3.99 (t, J = 13.8 Hz, 1H), 3.45 (dd, J = 15.4, 4.1 Hz, 1H), 3.31-3.30 (m, 1H), 3.14 (s, 3H), 2.78 (s, 3H), 2.09-2.00 (m, 1H), 1.80-1.59 (m, 5H), 1.26- 1.11 (m, 3H), 1.08-0.92 (m, 2H). |

The following compound was prepared using a procedure analogous to that used for Ex.401 starting from (R)-2-chloro-5-(7-chloro-3-cyclohexyl-2-methyl-1,1-dioxido-2,3,4,5-tetrahydrobenzo[f][1,2,5]thiadiazepin-8-yl)benzoic acid:

| Example | Structure | MS |
|---|---|---|
| 477 | 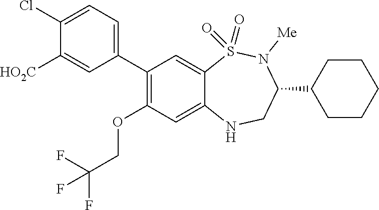 [SMILES: CN1[C@H](C2CCCCC2)CNc2cc(OCC(F)(F)F)c(-c3ccc(Cl)c(C(=O)O)c3)cc2S1(=O)=O] | [M + H]⁺ 547.3 |

### Ex.478 and Ex.479: Synthesis of (R)-2-(((benzyloxy)carbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)benzoic acid and (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-2-(((2-methoxyethoxy)carbonyl)amino)benzoic acid

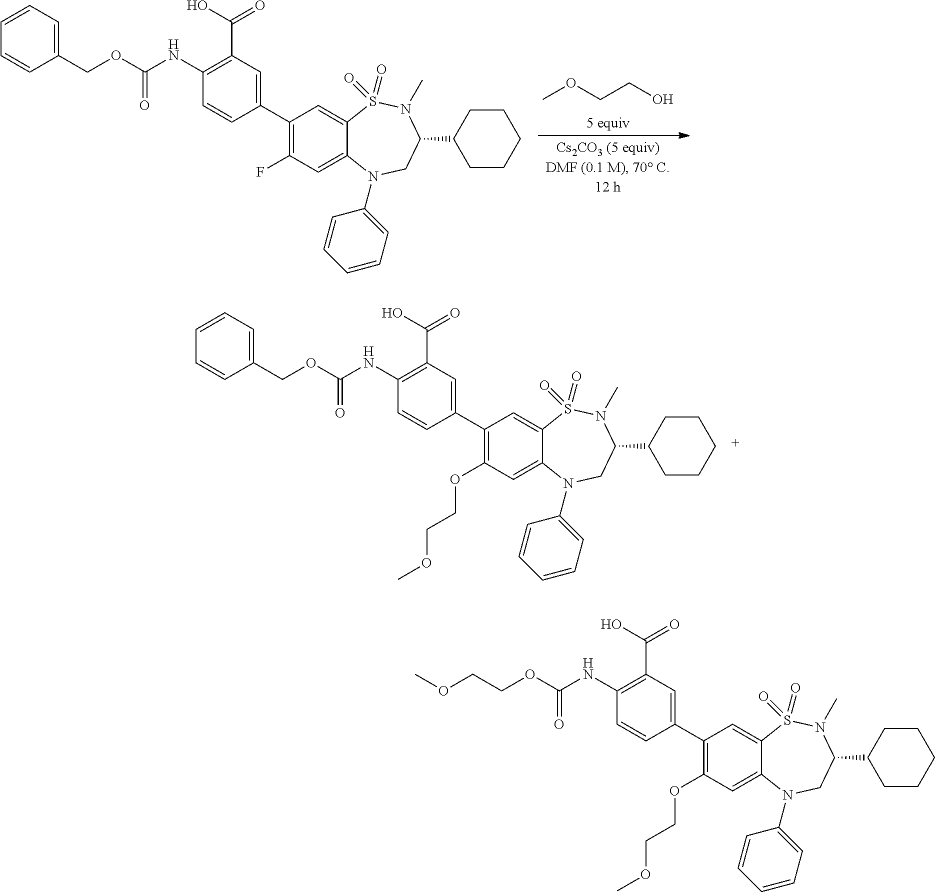

In a 2 mL vial equipped with a stir bar, (R)-2-(((benzyloxy)carbonyl)amino)-5-(3-cyclohexyl-7-fluoro-2-methyl-1,1-dioxido-5-phenyl-2,3,4,5-tetrahydrobenzo[f][1,2,5]thiadiazepin-8-yl) benzoic acid (15.0 mg, 1.0 equiv.) was dissolved in DMF (0.23 mL), followed by addition of 2-methoxyethanol (8.7 mg, 5.0 equiv., CAS #109-86-4) and cesium carbonate (37.2 mg, 5.0 equiv.). Reaction mixture was stirred 12 h at 70° C. in a heating block. The reaction was quenched with formic acid (0.2 mL) and concentrated under vacuum. The crude residue was re-dissolved in 2.0 mL of dimethyl sulfoxide, passed through a 0.45 μm syringe filter, and purified by RPHPLC to afford (R)-2-(((benzyloxy)carbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)benzoic acid (11.0 mg, 67.6% yield, ESI MS m/z=714.1 [M+H]⁺) and (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-2-(((2-methoxyethoxy) carbonyl)amino)benzoic acid (5.0 mg, 32.2% yield, ESI MS m/z=682.2 [M+H]⁺).

| Example | Structure | MS |
|---|---|---|
| 478 | 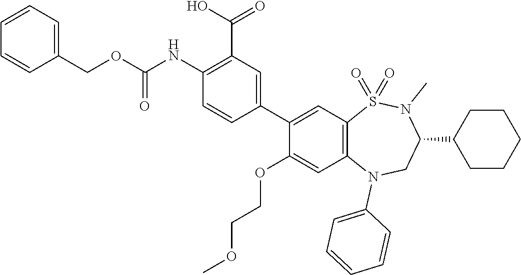 [SMILES: COCCOc1cc2c(cc1-c1ccc(NC(=O)OCc3ccccc3)c(C(=O)O)c1)S(=O)(=O)N(C)[C@H](C1CCCCC1)CN2c1ccccc1] | [M + H]⁺ 714.1 |
| 479 | 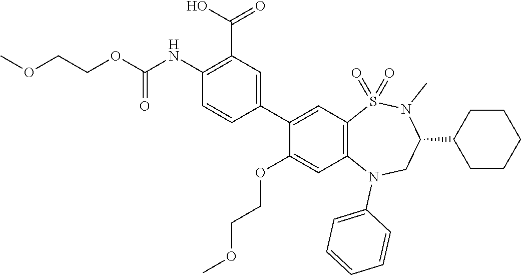 [SMILES: COCCOC(=O)Nc1ccc(-c2cc3c(cc2OCCOC)N(c2ccccc2)C[C@@H](C2CCCCC2)N(C)S3(=O)=O)cc1C(=O)O] | [M + H]⁺ 682.2 |

The following compounds were prepared using a procedure analogous to that described for Ex.478:

| Example | Structure | MS |
|---|---|---|
| 480 | 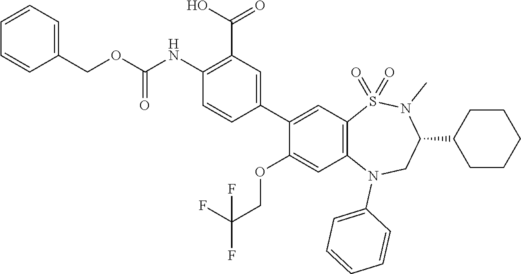 [SMILES: CN1[C@H](C2CCCCC2)CN(c2ccccc2)c2cc(OCC(F)(F)F)c(-c3ccc(NC(=O)OCc4ccccc4)c(C(=O)O)c3)cc2S1(=O)=O] | [M + H]⁺ 738.1 |
| 481 | 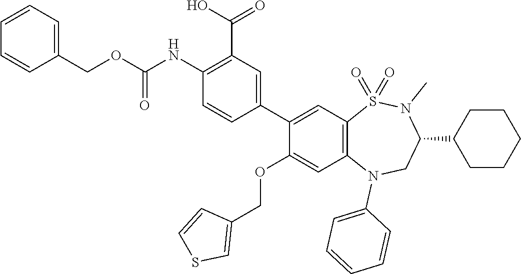 [SMILES: CN1[C@H](C2CCCCC2)CN(c2ccccc2)c2cc(OCc3ccsc3)c(-c3ccc(NC(=O)OCc4ccccc4)c(C(=O)O)c3)cc2S1(=O)=O] | [M + H]⁺ 752.2 |
| 482 | 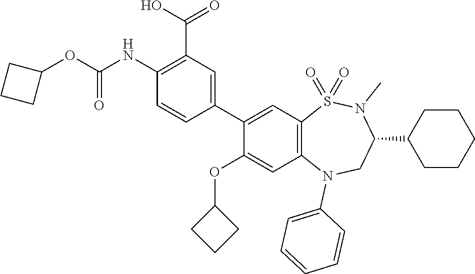 [SMILES: CN1[C@H](C2CCCCC2)CN(c2ccccc2)c2cc(OC3CCC3)c(-c3ccc(NC(=O)OC4CCC4)c(C(=O)O)c3)cc2S1(=O)=O] | [M + H]⁺ 674.3 |

### Ex.483: Synthesis of(R)-5-(3-cyclohexyl-7-(3,3-dimethylcyclobutoxy)-2-methyl-1,1-dioxido-5-phenyl-2,3,4,5-tetrahydrobenzo[1,2,5]thiadiazepin-8-yl)-3-fluorothiophene-2-carboxylic acid

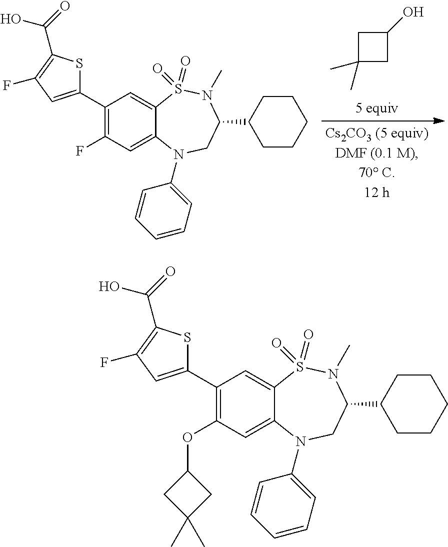

In a 2 mL vial equipped with a stir bar, (R)-5-(3-cyclohexyl-7-fluoro-2-methyl-1,1-dioxido-5-phenyl-2,3,4,5-tetrahydrobenzo[f][1,2,5]thiadiazepin-8-yl)-3-fluorothiophene-2-carboxylic acid (15 mg, 1.0 equiv.) was dissolved in DMF (0.285 ml), followed by addition of 3,3-dimethylcyclobutanol (14.1 mg, 5.0 equiv., CAS #54166-17-5) and cesium carbonate (45.9 mg, 5.0 equiv.) at room temperature. Then, reaction mixture was stirred 12 h at 70° C. in a heating block. Reaction progress was monitored through LC-MS. After 12 h, the reaction was quenched with formic acid (0.2 mL) and concentrated under vacuum. The crude residue was re-dissolved in 2.0 mL of dimethyl sulfoxide, passed through a 0.45 m syringe filter, and purified by RPTIPLC to afford (R)-5-(3-cyclohexyl-7-(3,3-dimethylcyclobutoxy)-2-methyl-1,1-dioxido-dioxido-5-phenyl-2,3,4,5-tetrahydrobenzo[f][1,2,5]thiadiazepin-8-yl)-3-fluorothiophene-2-carboxylic acid, Ex.483 (14.0 mg, 81% yield). ESI MS m/z=613.2 [M+H]⁺.

| Example | Structure | MS | NMR |
|---|---|---|---|
| 483 | 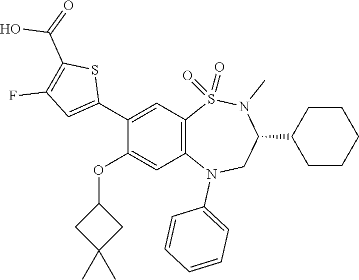 [SMILES: CN1[C@H](C2CCCCC2)CN(c2ccccc2)c2cc(OC3CC(C)(C)C3)c(-c3cc(F)c(C(=O)O)s3)cc2S1(=O)=O] | [M + H]⁺ 613.2 | ¹H NMR (400 MHz, DMSO-d6) δ 13.25 (br, 1H), 8.12 (s, 1H), 7.67 (s, 1H), 7.31 (t, J = 7.7 Hz, 2H), 7.19-6.81 (m, 3H), 6.59-6.40 (m, 1H), 4.98-4.55 (m, 1H), 4.33 (d, J = 15.9 Hz, 1H), 3.87-3.66 (m, 1H), 2.68 (s, 3H), 2.23-1.43 (m, 11H), 1.33-0.86 (m, 5H), 1.09 (d, J = 34.5 Hz, 12H). |

The following compounds were prepared using a procedure analogous to that described for Ex.483:

| Example | Structure | MS | NMR |
|---|---|---|---|
| 484 | 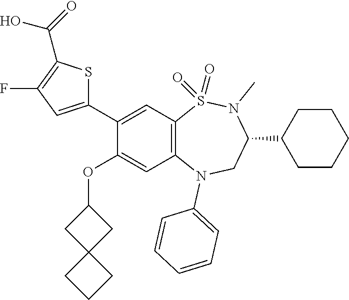 [SMILES: CN1[C@H](C2CCCCC2)CN(c2ccccc2)c2cc(OC3CC4(CCC4)C3)c(-c3cc(F)c(C(=O)O)s3)cc2S1(=O)=O] | [M + H]⁺ 625.2 | |
| 485 | 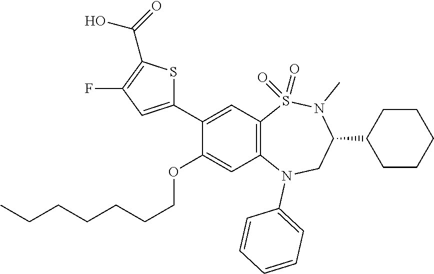 [SMILES: CCCCCCCOc1cc2c(cc1-c1cc(F)c(C(=O)O)s1)S(=O)(=O)N(C)[C@H](C1CCCCC1)CN2c1ccccc1] | [M + H]⁺ 629.3 | |
| 486 | 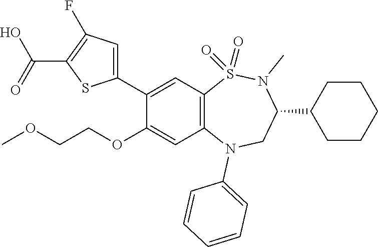 [SMILES: COCCOc1cc2c(cc1-c1cc(F)c(C(=O)O)s1)S(=O)(=O)N(C)[C@H](C1CCCCC1)CN2c1ccccc1] | [M + H]⁺ 589.2 | ¹H NMR (500 MHz, DMSO-d6) δ 13.24 (br, 1H), 8.12 (s, 1H), 7.67 (s, 1H), 7.28 (t, J = 7.8 Hz, 2H), 7.06 - 6.82 (m, 4H), 4.36 (d, J = 16.0 Hz, 1H), 4.26 - 4.16 (m, 1H), 4.17 - 4.02 (m, 1H), 3.68 (t, J = 4.6 Hz, 2H), 3.29 (s, 3H), 2.63 (s, 3H), 2.04 - 1.85 (m, 2H), 1.78 - 1.51 (m, 4H), 1.35 - 0.87 (m, 5H). |
| 487 | 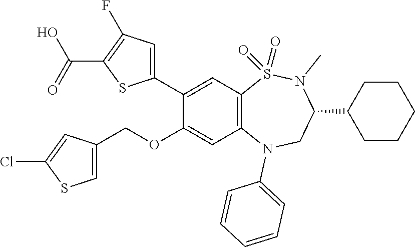 [SMILES: CN1[C@H](C2CCCCC2)CN(c2ccccc2)c2cc(OCc3csc(Cl)c3)c(-c3cc(F)c(C(=O)O)s3)cc2S1(=O)=O] | [M + H]⁺ 661.1 | |
| 488 | 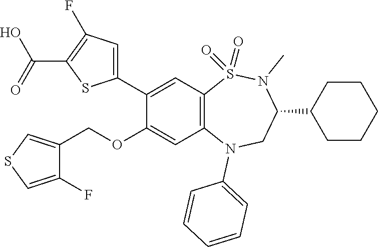 [SMILES: CN1[C@H](C2CCCCC2)CN(c2ccccc2)c2cc(OCc3cscc3F)c(-c3cc(F)c(C(=O)O)s3)cc2S1(=O)=O] | [M + H]⁺ 645.2 | |
| 489 | 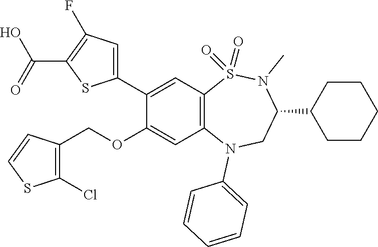 [SMILES: CN1[C@H](C2CCCCC2)CN(c2ccccc2)c2cc(OCc3ccsc3Cl)c(-c3cc(F)c(C(=O)O)s3)cc2S1(=O)=O] | [M + H]⁺ 661.2 | |
| 490 | 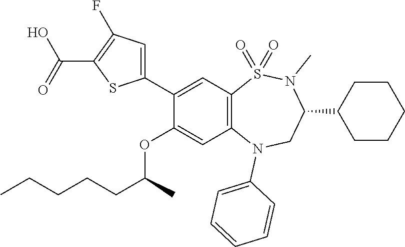 [SMILES: CCCCC[C@H](C)Oc1cc2c(cc1-c1cc(F)c(C(=O)O)s1)S(=O)(=O)N(C)[C@H](C1CCCCC1)CN2c1ccccc1] | [M + H]⁺ 629.3 | |
| 491 | 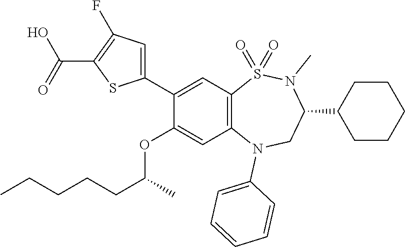 [SMILES: CCCCC[C@@H](C)Oc1cc2c(cc1-c1cc(F)c(C(=O)O)s1)S(=O)(=O)N(C)[C@H](C1CCCCC1)CN2c1ccccc1] | [M + H]⁺ 629.3 | |
| 492 | 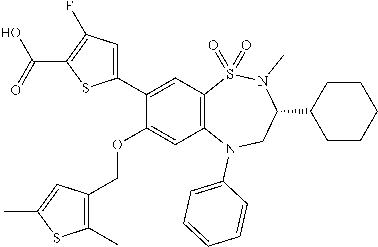 [SMILES: Cc1cc(COc2cc3c(cc2-c2cc(F)c(C(=O)O)s2)S(=O)(=O)N(C)[C@H](C2CCCCC2)CN3c2ccccc2)c(C)s1] | [M + H]⁺ 655.3 | |
| 493 | 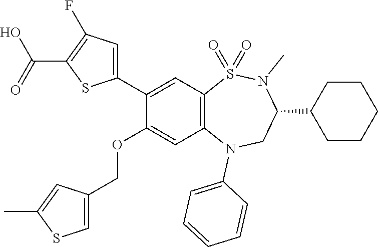 [SMILES: Cc1cc(COc2cc3c(cc2-c2cc(F)c(C(=O)O)s2)S(=O)(=O)N(C)[C@H](C2CCCCC2)CN3c2ccccc2)cs1] | [M + H]⁺ 641.2 | |
| 494 | 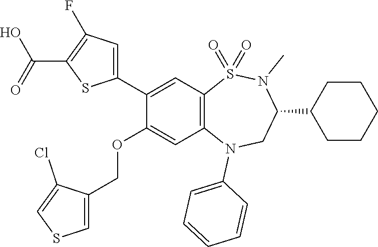 [SMILES: CN1[C@H](C2CCCCC2)CN(c2ccccc2)c2cc(OCc3cscc3Cl)c(-c3cc(F)c(C(=O)O)s3)cc2S1(=O)=O] | [M + H]⁺ 661.1 | |
| 495 | 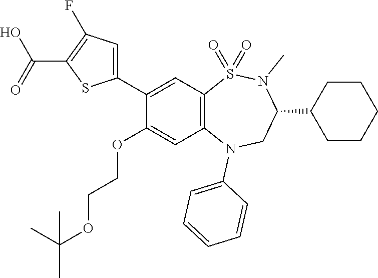 [SMILES: CN1[C@H](C2CCCCC2)CN(c2ccccc2)c2cc(OCCOC(C)(C)C)c(-c3cc(F)c(C(=O)O)s3)cc2S1(=O)=O] | [M + H]⁺ 631.2 | |
| 496 | 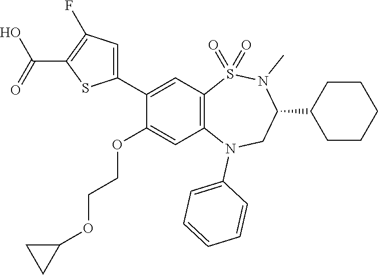 [SMILES: CN1[C@H](C2CCCCC2)CN(c2ccccc2)c2cc(OCCOC3CC3)c(-c3cc(F)c(C(=O)O)s3)cc2S1(=O)=O] | [M + H]⁺ 615.2 | |
| 497 | 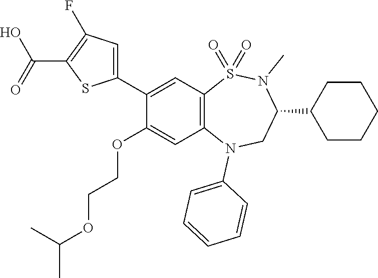 [SMILES: CC(C)OCCOc1cc2c(cc1-c1cc(F)c(C(=O)O)s1)S(=O)(=O)N(C)[C@H](C1CCCCC1)CN2c1ccccc1] | [M + H]⁺ 617.3 | |
| 498 | 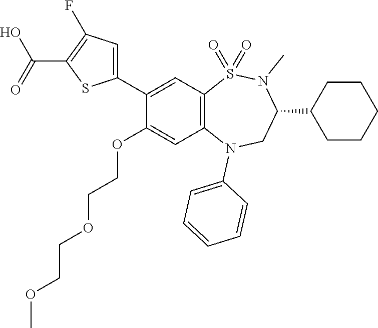 [SMILES: COCCOCCOc1cc2c(cc1-c1cc(F)c(C(=O)O)s1)S(=O)(=O)N(C)[C@H](C1CCCCC1)CN2c1ccccc1] | [M + H]⁺ 633.2 | |
| 499 | 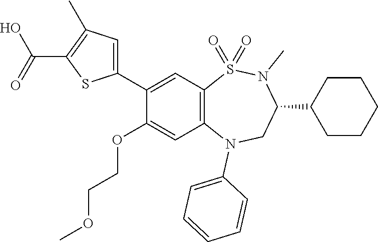 [SMILES: COCCOc1cc2c(cc1-c1cc(C)c(C(=O)O)s1)S(=O)(=O)N(C)[C@H](C1CCCCC1)CN2c1ccccc1] | [M + H]⁺ 585.3 | |
| 500 | 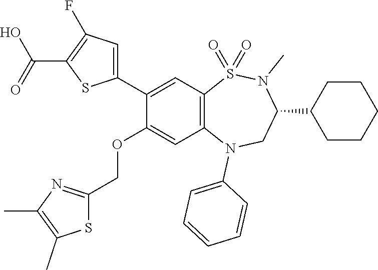 [SMILES: Cc1nc(COc2cc3c(cc2-c2cc(F)c(C(=O)O)s2)S(=O)(=O)N(C)[C@H](C2CCCCC2)CN3c2ccccc2)sc1C] | [M + H]⁺ 656.2 | |
| 501 | 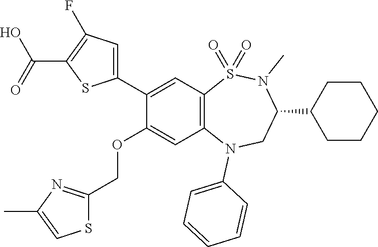 [SMILES: Cc1csc(COc2cc3c(cc2-c2cc(F)c(C(=O)O)s2)S(=O)(=O)N(C)[C@H](C2CCCCC2)CN3c2ccccc2)n1] | [M + H]⁺ 642.3 | |
| 502 | 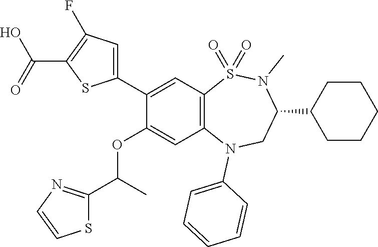 [SMILES: CC(Oc1cc2c(cc1-c1cc(F)c(C(=O)O)s1)S(=O)(=O)N(C)[C@H](C1CCCCC1)CN2c1ccccc1)c1nccs1] | [M + H]⁺ 642.3 | |
| 503 | 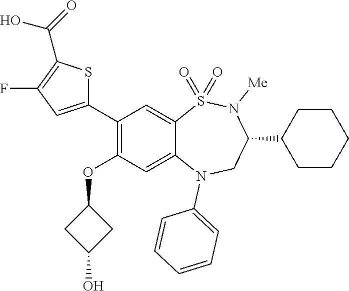 [SMILES: CN1[C@H](C2CCCCC2)CN(c2ccccc2)c2cc(O[C@H]3C[C@H](O)C3)c(-c3cc(F)c(C(=O)O)s3)cc2S1(=O)=O] | [M + H]⁺ 601.2 | |
| 504 | 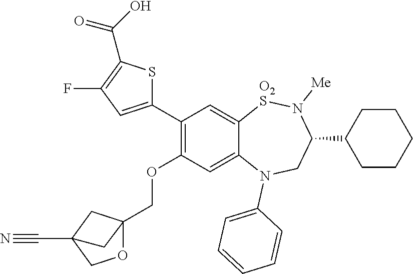 [SMILES: CN1[C@H](C2CCCCC2)CN(c2ccccc2)c2cc(OCC34CC(C#N)(CO3)C4)c(-c3cc(F)c(C(=O)O)s3)cc2S1(=O)=O] | [M + H]⁺ 652.2 | |
| 505 | 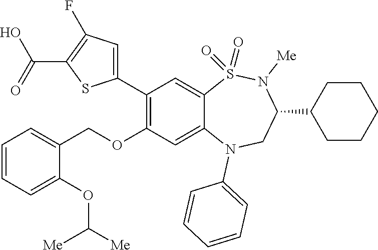 [SMILES: CC(C)Oc1ccccc1COc1cc2c(cc1-c1cc(F)c(C(=O)O)s1)S(=O)(=O)N(C)[C@H](C1CCCCC1)CN2c1ccccc1] | [M + H]⁺ 679.1 | |
| 506 | 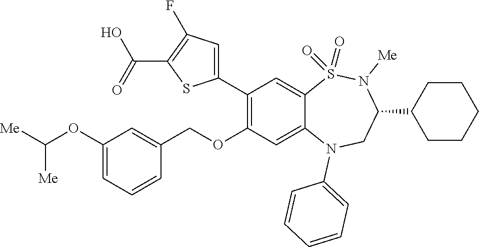 [SMILES: CC(C)Oc1cccc(COc2cc3c(cc2-c2cc(F)c(C(=O)O)s2)S(=O)(=O)N(C)[C@H](C2CCCCC2)CN3c2ccccc2)c1] | [M + H]⁺ 679.1 | |
| 507 | 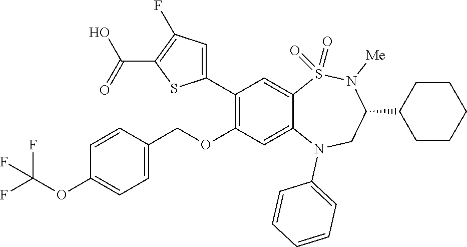 [SMILES: CN1[C@H](C2CCCCC2)CN(c2ccccc2)c2cc(OCc3ccc(OC(F)(F)F)cc3)c(-c3cc(F)c(C(=O)O)s3)cc2S1(=O)=O] | [M + H]⁺ 705.2 | |
| 508 | 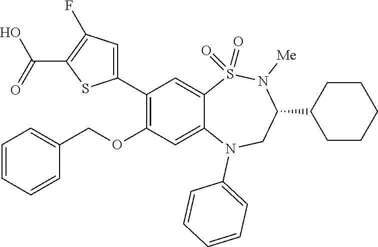 [SMILES: CN1[C@H](C2CCCCC2)CN(c2ccccc2)c2cc(OCc3ccccc3)c(-c3cc(F)c(C(=O)O)s3)cc2S1(=O)=O] | [M + H]⁺ 621.3 | |
| 509 | 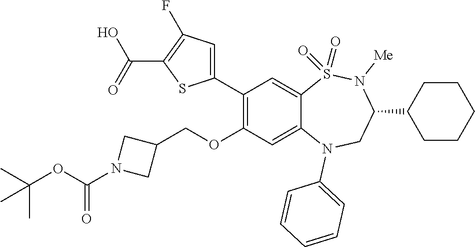 [SMILES: CN1[C@H](C2CCCCC2)CN(c2ccccc2)c2cc(OCC3CN(C(=O)OC(C)(C)C)C3)c(-c3cc(F)c(C(=O)O)s3)cc2S1(=O)=O] | [M + H]⁺ 700.5 | |
| 510 | 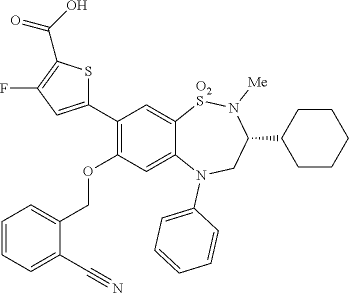 [SMILES: CN1[C@H](C2CCCCC2)CN(c2ccccc2)c2cc(OCc3ccccc3C#N)c(-c3cc(F)c(C(=O)O)s3)cc2S1(=O)=O] | [M + H]⁺ 646.3 | |
| 511 | 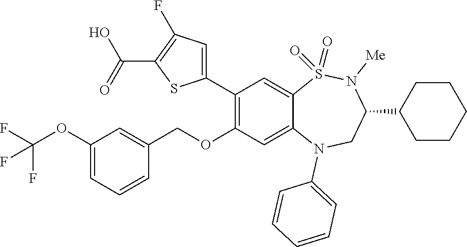 [SMILES: CN1[C@H](C2CCCCC2)CN(c2ccccc2)c2cc(OCc3cccc(OC(F)(F)F)c3)c(-c3cc(F)c(C(=O)O)s3)cc2S1(=O)=O] | [M + H]⁺ 705.1 | |

Ex.512: Synthesis of(R)-5-(3-cyclohexyl-2-methyl-1,1-dioxido-5-phenyl-7-(2,2,2-trifluoroethoxy)-2,3,4,5-tetrahydrobenzo[f][1,2,5]thiadiazepin-8-yl)-3-methylthiophene-2-carboxylic acid

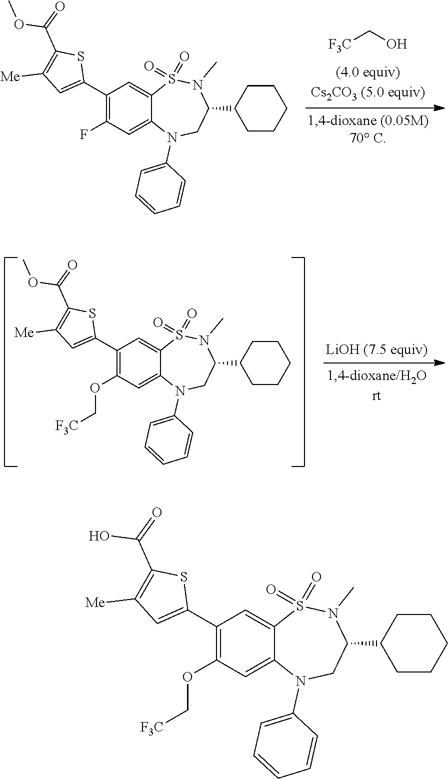

Step 1

In a 4 mL vial equipped with a stir bar, methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate (25.0 mg, 0.046 mmol, 1.0 equiv) was combined neat with cesium carbonate (75.0 mg, 0.23 mmol, 5.0 equiv). Next, 2,2,2-trifluoroethan-1-ol (18.4 mg, 0.184 mmol, 4.0 equiv) was added as a solution in 1,4-dioxane (0.92 mL, 0.05M). The reaction mixture was then heated in a sealed vial at 70° C. for 19 h. At this time, LCMS analysis indicated high conversion to methyl (R)-5-(3-cyclohexyl-2-methyl-1,1-dioxido-5-phenyl-7-(2,2,2-trifluoroethoxy)-2,3,4,5-tetrahydrobenzo[f][1,2,5]thiadiazepin-8-yl)-3-methylthiophene-2-carboxylate, and the reaction mixture was cooled to room temperature.

Step 2

Water (0.25 mL) was added to the mixture formed above in step 2, followed by lithium hydroxide (8.3 mg, 0.346 mmol, 7.5 equiv). The resulting mixture was stirred at room temperature for 48 h. Upon completion, as determined by LCMS analysis, the reaction mixture was quenched by the addition of formic acid (0.25 mL) and purified by RPHPLC. Lyophilization of the isolated product from a mixture of acetonitrile and water afforded (R)-5-(3-cyclohexyl-2-methyl-1,1-dioxido-5-phenyl-7-(2,2,2-trifluoroethoxy)-2,3,4,5-tetrahydrobenzo[f][1,2,5]thiadiazepin-8-yl)-3-methylthiophene-2-carboxylic acid, Ex.512 (4.21 mg, 15%). ESI MS m/z=609.2 [M+H]⁺.

| Example | Structure | MS |
|---|---|---|
| 512 | 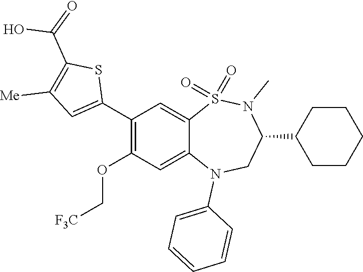 [SMILES: Cc1cc(-c2cc3c(cc2OCC(F)(F)F)N(c2ccccc2)C[C@@H](C2CCCCC2)N(C)S3(=O)=O)sc1C(=O)O] | [M + H]⁺ 609.2 |

Ex.513: Synthesis of (R)-5-(7-cyclobutoxy-3-cyclohexyl-2-methyl-1,1-dioxido-5-phenyl-2,3,4,5-tetrahydrobenzo[f][1,2,5]thiadiazepin-8-yl)-3-methylthiophene-2-carboxylic acid

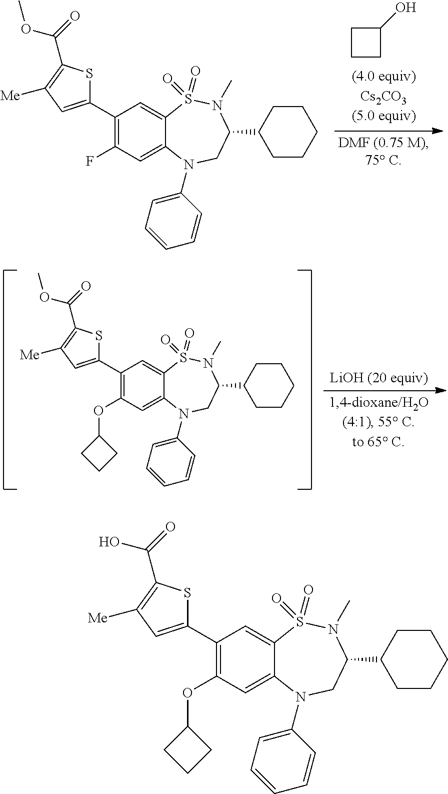

Step 1

In a 4 mL vial equipped with a stir bar, methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate (20.0 mg, 0.037 mmol, 1.0 equiv) was combined neat with cesium carbonate (60.0 mg, 0.184 mmol, 5.0 equiv). Next, cyclobutanol (10.6 mg, 0.147 mmol, 4.0 equiv) was added as a solution in N,N-dimethylformamide (0.49 mL). The resulting mixture was heated at 75° C. until LCMS analysis indicated full conversion to methyl (R)-5-(7-cyclobutoxy-3-cyclohexyl-2-methyl-1,1-dioxido-5-phenyl-2,3,4,5-tetrahydrobenzo[f][1,2,5]thiadiazepin-8-yl)-3-methylthiophene-2-carboxylate. Upon cooling to room temperature, the reaction mixture was concentrated and used directly in the next step.

Step 2

The crude residue produced in step 1 was dissolved in a mixture of 1,4-dioxane (1.0 mL) and water (0.25 mL). Lithium hydroxide (17.7 mg, 0.737 mmol, 20.0 equiv) was added and the reaction mixture was heated at 55° C. for 4 h. The mixture was then heated at 65° C. until LCMS analysis indicated full consumption of the starting material. Upon cooling to room temperature, the reaction was quenched by the addition of formic acid (0.25 mL), passed through a 0.45 micron syringe filter using N,N-dimethylformamide to rinse, and purified by RPHPLC. Lyophilization of the isolated product from a mixture of acetonitrile and water afforded (R)-5-(7-cyclobutoxy-3-cyclohexyl-2-methyl-1,1-dioxido-5-phenyl-2,3,4,5-tetrahydrobenzo[f][1,2,5]thiadiazepin-8-yl)-3-methylthiophene-2-carboxylic acid, Ex.513 (1.93 mg, 9%). ESI MS m/z=581.2 Example 514 was produced using a procedure analogous to that used for Ex.513.

| Example | Structure | MS |
|---|---|---|
| 513 | 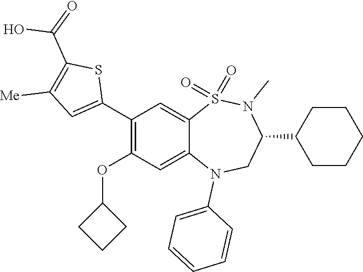 [SMILES: Cc1cc(-c2cc3c(cc2OC2CCC2)N(c2ccccc2)C[C@@H](C2CCCCC2)N(C)S3(=O)=O)sc1C(=O)O] | [M + H]⁺ 581.2 |
| 514 | 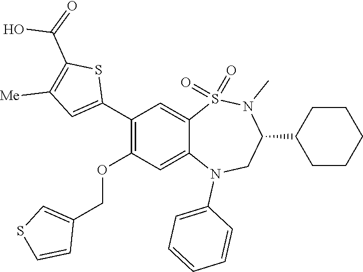 [SMILES: Cc1cc(-c2cc3c(cc2OCc2ccsc2)N(c2ccccc2)C[C@@H](C2CCCCC2)N(C)S3(=O)=O)sc1C(=O)O] | [M + H]⁺ 623.2 |

### Ex.515: Synthesis of (R)-3-((tert-butoxycarbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylic acid

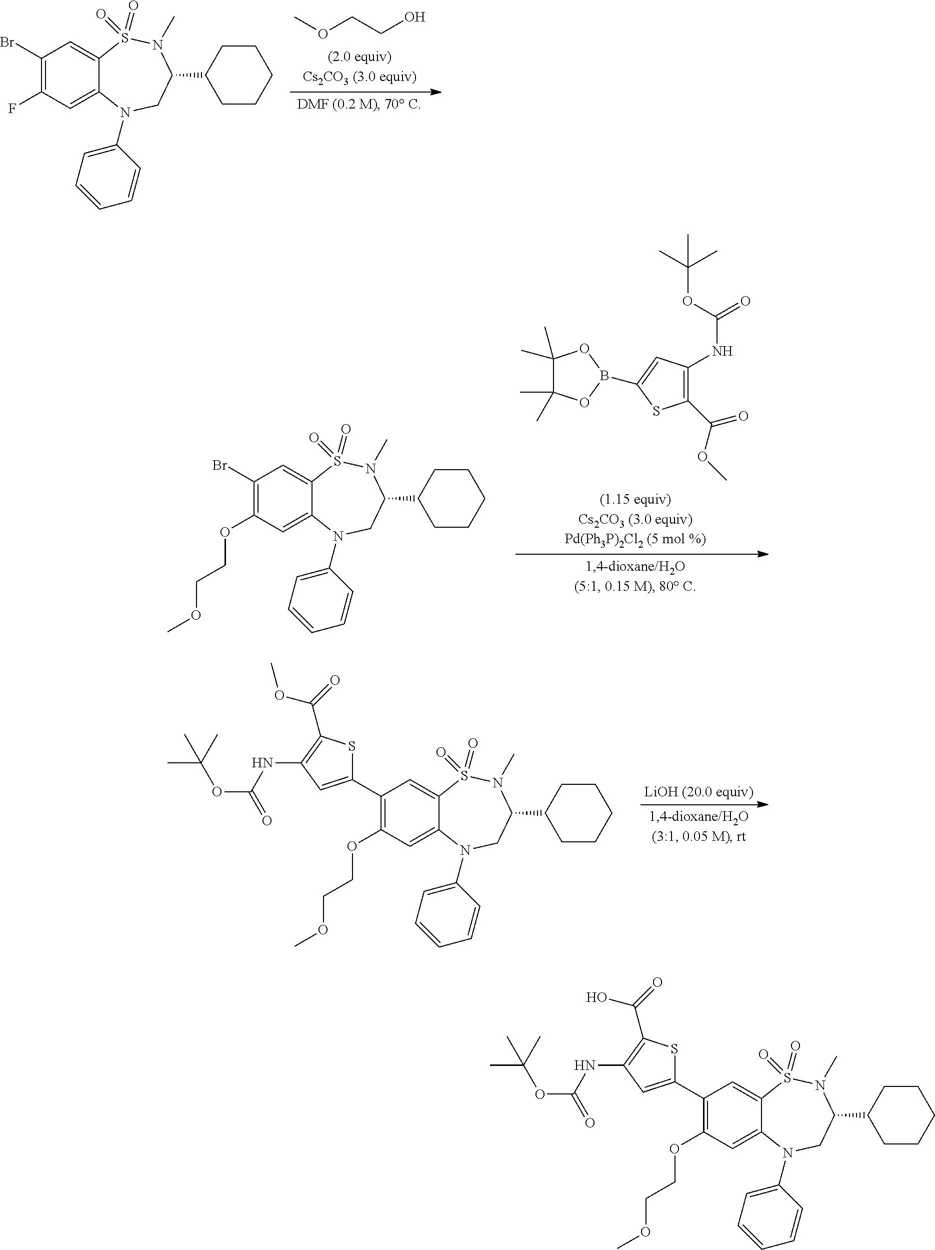

Step 1

In a 40 mL vial equipped with a stir bar, (R)-8-bromo-3-cyclohexyl-7-fluoro-2-methyl-5-phenyl-2,3,4,5-tetrahydrobenzo[f][1,2,5]thiadiazepine 1,1-dioxide (1.0 g, 2.14 mmol, 1.0 equiv) was combined with cesium carbonate (2.09 g, 6.42 mmol, 3.0 equiv) in N,N-dimethylformamide (10.7 mL, 0.2M). Next, 2-methoxyethan-1-ol (326 mg, 0.34 mL, 4.28 mmol, 2.0 equiv, CAS #109-86-4) was added. The mixture was then heated at 70° C. for 3 h, at which time LCMS analysis indicated full conversion of the starting material. Upon cooling to room temperature, the mixture was diluted with water and extracted with ethyl acetate. The combined organic layers were washed with brine and dried over magnesium sulfate. Purification by silica gel column chromatography (gradient elution, 0 to 20% ethyl acetate/cyclohexane) afforded (R)-8-bromo-3-cyclohexyl-7-(2-methoxyethoxy)-2-methyl-5-phenyl-2,3,4,5-tetrahydrobenzo[f][1,2,5]thiadiazepine 1,1-dioxide (1.0 g, 89%). ESI MS m/z=523.0 [M+H]⁺.

Step 2

In a 40 mL vial equipped with a stir bar, (R)-8-bromo-3-cyclohexyl-7-(2-methoxyethoxy)-2-methyl-5-phenyl-2,3,4,5-tetrahydrobenzo[f][1,2,5]thiadiazepine 1,1-dioxide (1.0 g, 1.91 mmol, 1.0 equiv) was combined neat with methyl 3-((tert-butoxycarbonyl)amino)-5-(4,4,5,5-tetramethyl-1,3,2-dioxaborolan-2-yl)thiophene-2-carboxylate (934 mg, 2.20 mmol, 1.15 equiv, CAS #2377606-49-8), cesium carbonate (1.87 g, 5.73 mmol, 3.0 equiv), and bis(triphenylphosphine)palladium(II) chloride (67.0 mg, 5 mol %) under a nitrogen atmosphere. Next, 1,4-dioxane (10.6 mL) and water (2.1 mL) were added, and the vial was sealed with electrical tape. The mixture was heated at 80° C. for 3 h. Upon cooling to room temperature, the reaction mixture was concentrated and purified by silica gel column chromatography (ethyl acetate/cyclohexane) to afford methyl (R)-3-((tert-butoxycarbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate (1.22 g, 91%). ESI MS m/z=599.9 [M-C₄H₈-CO₂+H]⁺.

Step 3

In a 1 dram vial equipped with a stir bar, (R)-3-((tert-butoxycarbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate (25.0 mg, 0.036 mmol, 1.0 equiv) was dissolved in a mixture of 1,4-dioxane (0.54 mL) and water (0.18 mL) and lithium hydroxide (17.1 mg, 0.71 mmol, 20.0 equiv) was administered. The mixture was stirred for 15 h and quenched with formic acid (0.25 mL) and passed through a 0.45 micron syringe filter using N,N-dimethylformamide to rinse before being purified by RPHPLC. Lyophilization of the isolated product from a mixture of acetonitrile and water afforded (R)-3-((tert-butoxycarbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylic acid, Ex.515 (13.6 mg, 55%) as a light yellow solid. ESI MS m/z=585.8 [M-C₄H₈-CO₂+H]⁺.

| Example | Structure | MS |
|---|---|---|
| 515 | 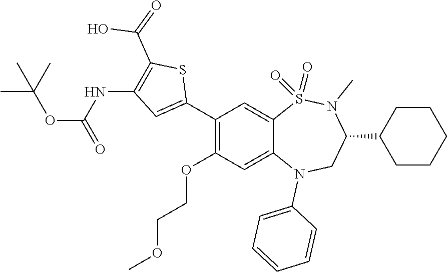 [SMILES: COCCOc1cc2c(cc1-c1cc(NC(=O)OC(C)(C)C)c(C(=O)O)s1)S(=O)(=O)N(C)[C@H](C1CCCCC1)CN2c1ccccc1] | [M + H]⁺ 585.8 |

### Ex.516: Synthesis of (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-3-((isobutoxycarbonyl)amino)thiophene-2-carboxylic acid

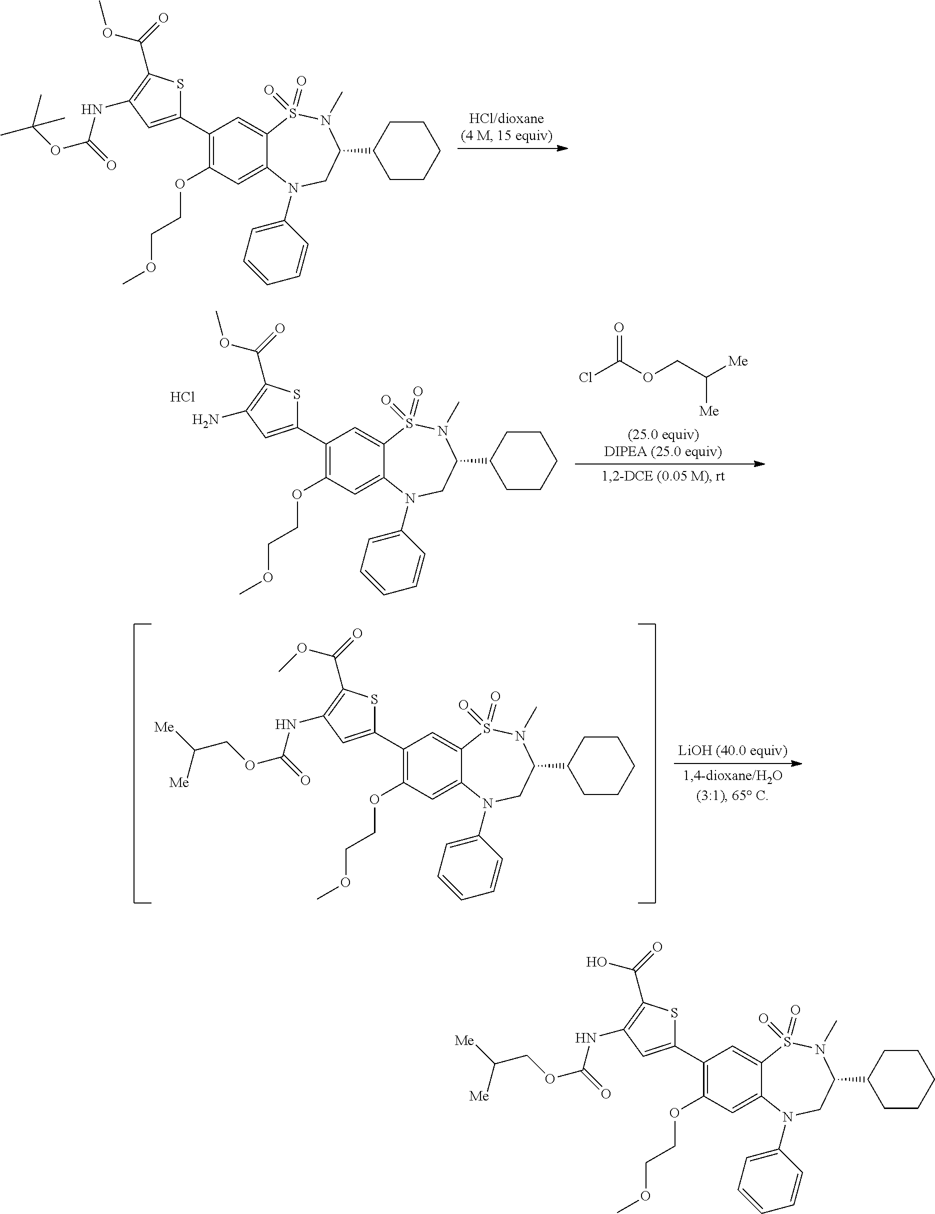

Step 1

Methyl (R)-3-((tert-butoxycarbonyl)amino)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate (442.0 mg, 0.63 mmol, 1.0 equiv) was treated with HCl in 1,4-dioxane (4M, 2.37 mL, 15.0 equiv). The reaction was stirred for 1.5 h at room temperature, at which time LCMS analysis indicated full consumption of the starting material. The mixture was concentrated to afford methyl (R)-3-amino-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate hydrochloride (402.0 mg theoretical) which was used in the subsequent step without purification. ESI MS m/z=600.2 [M+H]⁺.

Step 2

In a 1 dram vial equipped with a stir bar, (R)-3-amino-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate hydrochloride (35.0 mg, 0.055 mmol, 1.0 equiv) was dissolved in 1,2-dichloroethane (1.1 mL, 0.05M) and N,N-diisopropylethylamine (178.0 mg, 0.24 mL, 1.38 mmol, 25 equiv) was added, followed by isobutyl chloroformate (188.0 mg, 0.18 mL, 1.38 mmol, 25 equiv). The resulting mixture was stirred at room temperature for 11 h. The reaction mixture was then quenched with water (0.5 mL) and concentrated to afford the crude methyl (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-3-((isobutoxycarbonyl)amino)thiophene-2-carboxylate.

Step 3

The residue generated in step 2 above was dissolved in a mixture of 1,4-dioxane (0.90 mL) and water (0.30 mL). Lithium hydroxide (52.7 mg, 2.20 mmol, 40 equiv) was added, and the mixture was heated at 65° C. while conversion was monitored by LCMS analysis. Upon cooling to room temperature, the mixture was quenched with formic acid (0.25 mL) and passed through a 0.45 micron syringe filter using N,N-dimethylformamide to rinse before being purified by RPHPLC. Lyophilization of the isolated product from a mixture of acetonitrile and water afforded (R)-5-(3-cyclohexyl-7-(2-methoxyethoxy)-2-methyl-1,1-dioxido-dioxido-5-phenyl-2,3,4,5-tetrahydrobenzo[f][1,2,5]thiadiazepin-8-yl)-3-((isobutoxycarbonyl)amino)thiophene-2-carboxylic acid, Ex.516 as a light yellow solid (10.6 mg, 28%). ESI MS m/z=686.3 [M+H]⁺.

Example 517 was produced using a procedure analogous to that used for Ex.516.

| Example | Structure | MS |
|---|---|---|
| 516 | 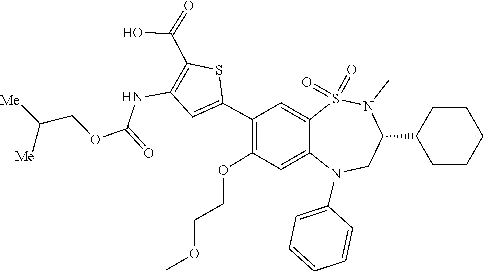 [SMILES: COCCOc1cc2c(cc1-c1cc(NC(=O)OCC(C)C)c(C(=O)O)s1)S(=O)(=O)N(C)[C@H](C1CCCCC1)CN2c1ccccc1] | [M + H]⁺ 686.3 |
| 517 | 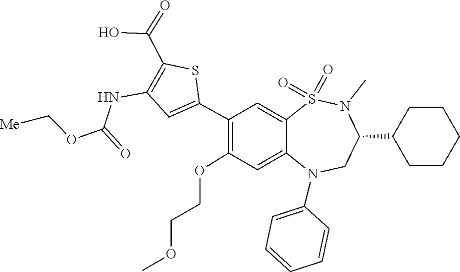 [SMILES: CCOC(=O)Nc1cc(-c2cc3c(cc2OCCOC)N(c2ccccc2)C[C@@H](C2CCCCC2)N(C)S3(=O)=O)sc1C(=O)O] | [M + H]⁺ 658.3 |

### Ex.518: Synthesis of (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-3-(((2-isopropoxyethoxy)carbonyl)amino)thiophene-2-carboxylic acid

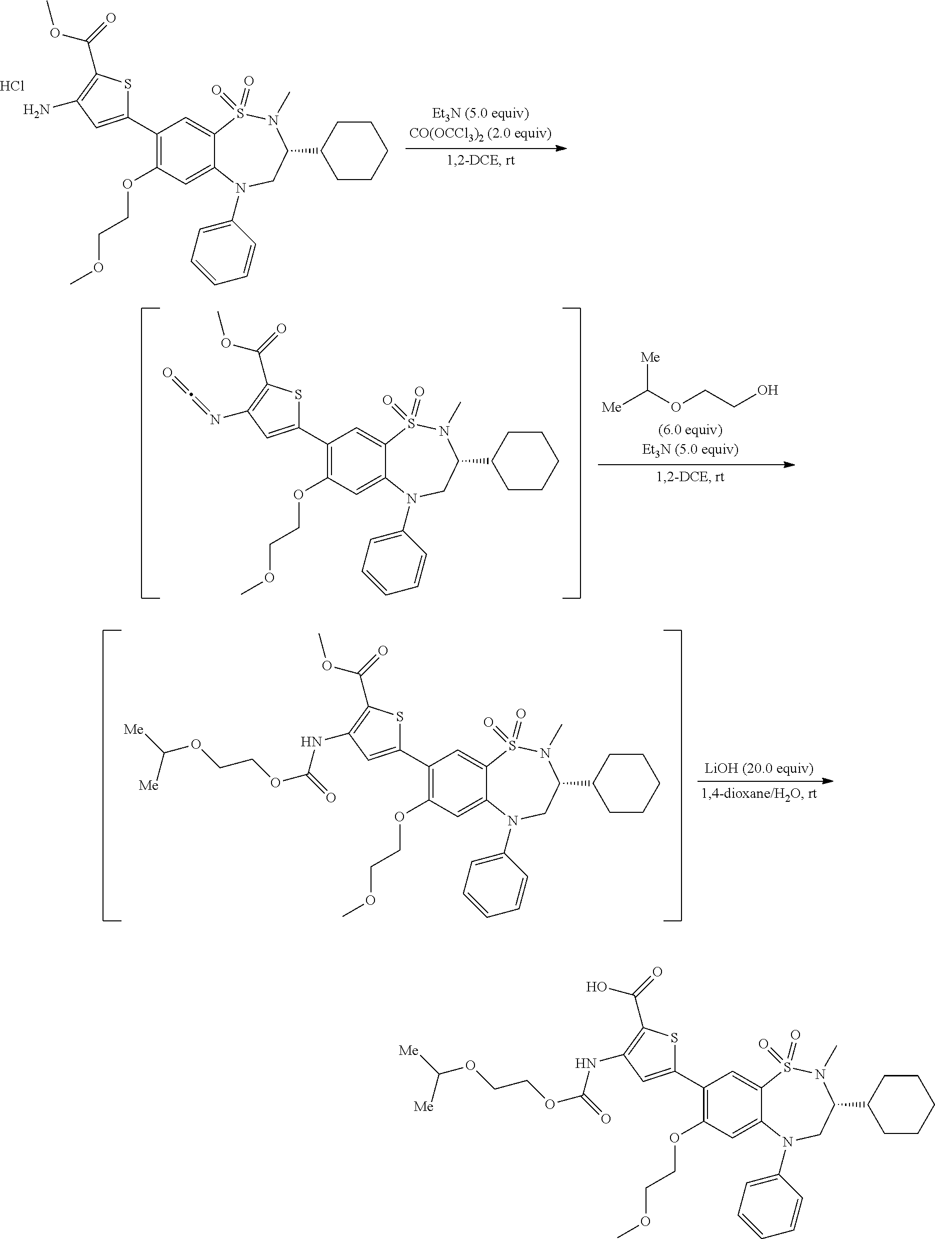

Step 1

In a 1 dram vial equipped with a stir bar, triphosgene (46.6 mg, 0.157 mmol, 2.0 equiv) was dissolved in 1,2-dichloroethane (0.5 mL). Triethylamine (39.8 mg, 0.055 mL, 0.39 mmol, 5.0 equiv) was added, followed by dropwise addition of methyl (R)-3-amino-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)thiophene-2-carboxylate hydrochloride (50.0 mg, 0.079 mmol, 1.0 equiv) as a solution in 1,2-dichloroethane (1.0 mL). The mixture was stirred for 15 min at room temperature.

Step 2

To the mixture generated in step 1 was added 2-isopropoxyethan-1-ol (49.1 mg, 0.47 mmol, 6.0 equiv) as a solution in 1,2-dichloroethane (0.5 mL). The mixture was stirred for 30 min and additional 2-isopropoxyethan-1-ol (49.1 mg, 0.47 mmol, 6.0 equiv) was added in 1,2-dichloroethane (0.5 mL), followed by the addition of triethylamine (39.8 mg, 0.055 mL, 0.39 mmol, 5.0 equiv). After 15 mi, LCMS analysis indicated full conversion to methyl (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-3-(((2-isopropoxyethoxy)carbonyl)amino)thiophene-2-carboxylate, and the reaction mixture was quenched with water (0.5 mL) and concentrated.

Step 3

The residue generated above in step 3 was dissolved in 1,4-dioxane (0.75 mL) and water (0.3 mL) and lithium hydroxide (37.6 mg, 1.57 mmol, 20.0 equiv) was added. The reaction mixture was stirred at room temperature until LCMS analysis indicated full conversion. The reaction was quenched with formic acid (0.25 mL) and passed through a 0.45 micron syringe filter before being purified by RPHPLC. Lyophilization of the isolated product from a mixture of acetonitrile and water afforded (R)-5-(3-cyclohexyl-7-(2-methoxyethoxy)-2-methyl-1,1-dioxido-5-phenyl-2,3,4,5-tetrahydrobenzo[f][1,2,5]thiadiazepin-8-yl)-3-(((2-isopropoxyethoxy)carbonyl)amino)thiophene-2-carboxylic acid, Ex.518 (12.9 mg, 22.9%) as a light yellow solid. ESI MS m/z=716.2 [M+H]⁺.

Example 519 was produced using a procedure analogous to that used for Ex.518.

| Example | Structure | MS |
|---|---|---|
| 518 | 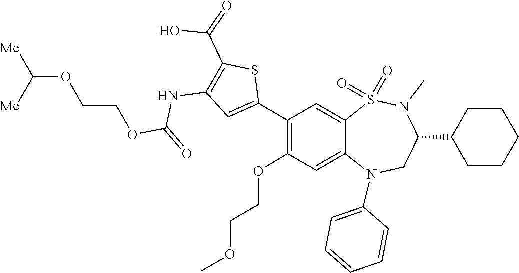 [SMILES: COCCOc1cc2c(cc1-c1cc(NC(=O)OCCOC(C)C)c(C(=O)O)s1)S(=O)(=O)N(C)[C@H](C1CCCCC1)CN2c1ccccc1] | [M + H]⁺ 716.2 |
| 519 | 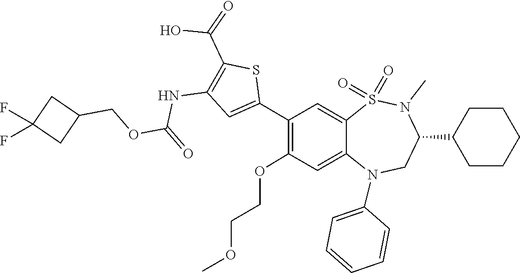 [SMILES: COCCOc1cc2c(cc1-c1cc(NC(=O)OCC3CC(F)(F)C3)c(C(=O)O)s1)S(=O)(=O)N(C)[C@H](C1CCCCC1)CN2c1ccccc1] | [M + H]⁺ 734.4 |

### Ex. 520: Synthesis of (R)-5-(3-butyl-2-methyl-1,1-dioxido-5-phenyl-7-(pyrrolidin-1-yl)-2,3,4,5-tetrahydrobenzo[f][1,2,5]thiadiazepin-8-yl)-2-fluorobenzoic acid

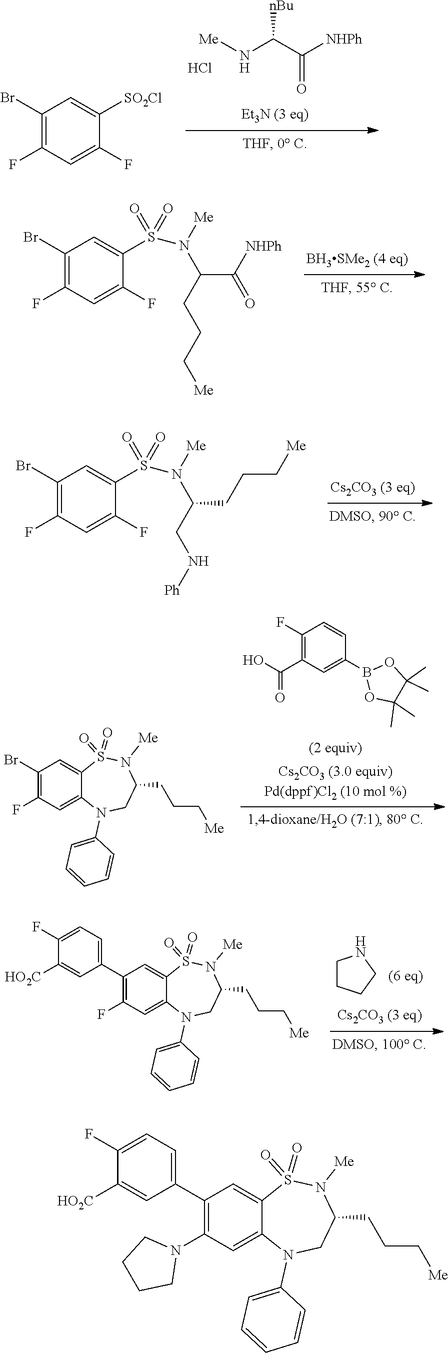

Step 1

To a stirred solution of (R)-2-(methylamino)-N-phenylhexanamide hydrochloride (2.202 g, 8.58 mmol) in THF (42.9 mL) was added 5-bromo-2,4-difluorobenzenesulfonyl chloride (2.50 g, 8.58 mmol) and Et₃N (3.59 mL, 25.7 mmol) at 0° C. and the reaction mixture was then stirred for 16 hours at room temperature. After completion of the reaction (monitored by LCMS), the reaction mixture was filtered through celite, washing with EtOAc. The solution was concentrated and the resulting crude product was purified by silica gel column chromatography (eluent: 0-20% EtOAc/cyclohexane) to afford ((R)-2-((5-bromo-2,4-difluoro-N-methylphenyl)sulfonamido)-N-phenylhexanamide (3.28 g, 6.90 mmol, 81% yield) as a white solid. ¹H NMR (400 MHz, Chloroform-d) δ 8.18 (t, J=7.3 Hz, 1H), 8.04 (s, 1H), 7.53 (d, J=7.8 Hz, 2H), 7.40-7.31 (m, 2H), 7.20-7.12 (m, 1H), 7.07 (dd, J=9.5, 7.8 Hz, 1H), 4.38 (t, J=7.5 Hz, 1H), 2.99 (d, J=1.5 Hz, 3H), 2.09-1.95 (m, 1H), 1.63-1.50 (m, 1H), 1.35-1.20 (m, 2H), 1.19-1.05 (m, 2H), 0.84 (t, J=7.3 Hz, 3H). ESI MS m/z=475.1 [M+H]⁺.

Step 2

To (R)-2-((5-bromo-2,4-difluoro-N-methylphenyl)sulfonamido)-N-phenylhexanamide (3.28 g, 6.90 mmol)) dissolved in THE (23.00 mL) was added BH₃-DMS complex (2.62 mL, 27.6 mmol). The mixture was then stirred at 55° C. overnight. Upon completion, the reaction was cooled to room temperature, slowly quenched with water, and diluted with EtOAc. The mixture was acidified with 1 N HCl (pH=1) and extracted with EtOAc. The combined organic layers were dried over Na₂SO₄, filtered, concentrated and the resulting crude product was purified by silica gel column chromatography (eluent: 0-20% EtOAc/cyclohexane) to afford ((R)-2-((5-bromo-2,4-difluoro-N-methylphenyl)sulfonamido)-N-phenylhexanamide (2.81 g, 6.09 mmol, 88% yield) as an oil. ¹H NMR (400 MHz, Chloroform-d) δ 8.10 (t, J=7.3 Hz, 1H), 7.24 (t, J=8.0 Hz, 2H), 7.00 (dd, J=9.5, 7.9 Hz, 1H), 6.88 (t, J=7.4 Hz, 1H), 6.72 (d, J=7.9 Hz, 2H), 4.08-3.97 (m, 1H), 3.29-3.15 (m, 2H), 2.91 (d, J=1.9 Hz, 3H), 1.66-1.38 (m, 1H), 1.28-1.18 (m, 2H), 1.18-1.01 (m, 3H), 0.83 (t, J=7.1 Hz, 3H). ESI MS m/z=461.2 [M+H]⁺.

Step 3

A mixture of (R)-5-bromo-2,4-difluoro-N-methyl-N-(1-(phenylamino)hexan-2-yl)benzenesulfonamide (2.81 g, 6.09 mmol) and cesium carbonate (5.95 g, 18.27 mmol) in DMSO (24.36 mL) was heated at 90° C. After 1 hour, the reaction was cooled to room temperature and diluted with EtOAc, then washed with H₂O (×5) and brine. The organic layer was dried over Na₂SO₄, filtered, concentrated, and purified by silica gel column chromatography (eluent: 0-10% EtOAc/cyclohexane) to afford (R)-8-bromo-3-butyl-7-fluoro-2-methyl-5-phenyl-2,3,4,5-tetrahydrobenzo[f][1,2,5]thiadiazepine 1,1-dioxide (1.26 g, 4.03 mmol, 66% yield). ¹H NMR (400 MHz, Chloroform-d) δ 8.15 (d, J=7.7 Hz, 1H), 7.35 (t, J=7.9 Hz, 2H), 7.09 (t, J=7.4 Hz, 1H), 7.02 (d, J=7.3 Hz, 2H), 6.75 (d, J=10.1 Hz, 1H), 4.05-3.98 (m, 1H), 3.89-3.78 (m, 1H), 3.77-3.67 (m, 1H), 2.81 (s, 3H), 1.50 (td, J=9.9, 8.9, 4.6 Hz, 2H), 1.42-1.30 (m, 4H), 0.94 (t, J=7.3 Hz, 3H). ESI MS m/z=441.1 [M+H]⁺.

Step 4

(R)-8-bromo-3-butyl-7-fluoro-2-methyl-5-phenyl-2,3,4,5-tetrahydrobenzo[f][1,2,5]thiadiazepine 1,1-dioxide (200 mg, 0.453 mmol), 2-fluoro-5-(4,4,5,5-tetramethyl-1,3,2-dioxaborolan-2-yl)benzoic acid (241 mg, 0.906 mmol), cesium carbonate (443 mg, 1.359 mmol), and PdCl₂(dppf) (33.2 mg, 0.045 mmol) were combined in vial equipped with a stir bar under nitrogen. Dioxane (3.94 mL) and Water (0.591 mL) were added, and the reaction was heated at 90° C. overnight. The reaction mixture was diluted with EtOAc and acidified with 1 N HCl, the aqueous layer was then extracted with EtOAc. The combined organic layers were dried over Na₂SO₄, filtered, concentrated, and purified by silica gel column chromatography (eluent: 0-25% EtOAc/cyclohexane) to afford (R)-5-(3-butyl-7-fluoro-2-methyl-1,1-dioxido-5-phenyl-2,3,4,5-tetrahydrobenzo[f][1,2,5]thiadiazepin-8-yl)-2-fluorobenzoic acid (203 mg, 0.41 mmol, 89% yield). ¹H NMR (400 MHz, DMSO-d₆) δ 13.45 (s, 1H), 8.08-7.99 (m, 1H), 7.97-7.92 (m, 1H), 7.92-7.85 (m, 1H), 7.47 (dd, J=10.6, 8.6 Hz, 1H), 7.28 (t, J=7.8 Hz, 2H), 7.20 (d, J=11.9 Hz, 1H), 6.98-6.85 (m, 3H), 4.13 (d, J=16.0 Hz, 1H), 3.79-3.72 (m, 1H), 3.57-3.53 (m, 1H), 2.64 (s, 3H), 1.58 (t, J=7.6 Hz, 2H), 1.41-1.27 (m, 4H), 0.91 (t, J=6.9 Hz, 3H). ESI MS m/z=501.2 [M+H]⁺.

Step 5

To a vial equipped with a stir bar with (S)-3-(3-butyl-7-fluoro-2-methyl-1,1-dioxido-5-phenyl-2,3,4,5-tetrahydrobenzo[f][1,2,5]thiadiazepin-8-yl)benzoic acid (20 mg, 0.041 mmol) and Cs₂CO₃ (67.5 mg, 0.207 mmol) in DMSO (0.414 mL) was added pyrrolidine (20.57 μl, 0.249 mmol) and the mixture was heated to 100° C. for 4-16 h. Upon completion, the reaction mixture was acidified with formic acid and the mixture was purified directly via preparative HPLC to afford (R)-5-(3-butyl-2-methyl-1,1-dioxido-5-phenyl-7-(pyrrolidin-1-yl)-2,3,4,5-tetrahydrobenzo[f][1,2,5]thiadiazepin-8-yl)-2-fluorobenzoic acid, Ex.520 (15 mg, 0.03 mmol, 66% yield) ¹H NMR (400 MHz, DMSO-d₆) δ 7.80 (dd, J=7.1, 2.5 Hz, 1H), 7.67-7.61 (m, 1H), 7.44 (s, 1H), 7.36 (dd, J=10.7, 8.5 Hz, 1H), 7.18 (t, J=7.8 Hz, 2H), 6.79-6.71 (m, 3H), 6.65 (s, 1H), 4.02 (d, J=16.2 Hz, 1H), 3.87-3.74 (m, 1H), 3.43-3.37 (m, 1H), 2.94-2.80 (m, 4H), 2.46 (s, 3H), 1.76-1.67 (m, 4H), 1.63-1.46 (m, 2H), 1.40-1.28 (m, J=7.1, 5.0 Hz, 4H), 0.94-0.86 (m, 3H). ESI MS m/z=552.4 [M+H]⁺.

Examples 521-557 were made using similar methods to those described above:

| Example | Structure | MS | NMR |
|---|---|---|---|
| 520 | 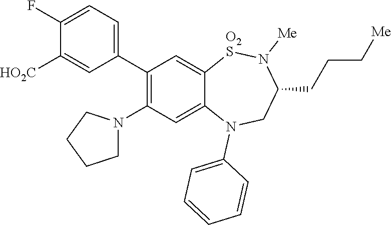 [SMILES: CCCC[C@@H]1CN(c2ccccc2)c2cc(N3CCCC3)c(-c3ccc(F)c(C(=O)O)c3)cc2S(=O)(=O)N1C] | [M + H]⁺ 552.4 | ¹H NMR (400 MHz, DMSO-d₆) δ 7.80 (dd, J = 7.1, 2.5 Hz, 1H), 7.67 - 7.61 (m, 1H), 7.44 (s, 1H), 7.36 (dd, J = 10.7, 8.5 Hz, 1H), 7.18 (t, J = 7.8 Hz, 2H), 6.79 - 6.71 (m, 3H), 6.65 (s, 1H), 4.02 (d, J = 16.2 Hz, 1H), 3.87 - 3.74 (m, 1H), 3.43 - 3.37 (m, 1H), 2.94 - 2.80 (m, 4H), 2.46 (s, 3H), 1.76 - 1.67 (m, 4H), 1.63 - 1.46 (m, 2H), 1.40 - 1.28 (m, J = 7.1, 5.0 Hz, 4H), 0.94 - 0.86 (m, 3H). |
| 521 | 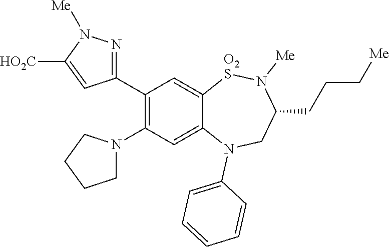 [SMILES: CCCC[C@@H]1CN(c2ccccc2)c2cc(N3CCCC3)c(-c3cc(C(=O)O)n(C)n3)cc2S(=O)(=O)N1C] | [M + H]⁺ 538.377 | ¹H NMR (400 MHz, DMSO-d₆) δ 7.59 (s, 1H), 7.22 - 7.15 (m, 2H), 6.88 (s, 1H), 6.73 (dd, J = 8.2, 5.5 Hz, 3H), 6.60 (s, 1H), 4.13 (s, 3H), 4.02 (d, J = 16.5 Hz, 1H), 3.89 - 3.79 (m, 1H), 3.40 (s, 1H), 3.09 - 2.77 (m, 4H), 2.44 (s, 3H), 1.81 - 1.72 (m, 4H), 1.55 (ddt, J = 24.8, 10.1, 6.0 Hz, 2H), 1.35 (ddt, J = 14.9, 10.7, 6.3 Hz, 4H), 0.91 (td, J = 6.9, 0.0 Hz, 3H). |
| 522 | 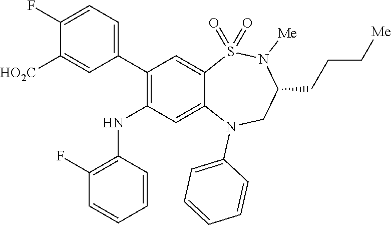 [SMILES: CCCC[C@@H]1CN(c2ccccc2)c2cc(Nc3ccccc3F)c(-c3ccc(F)c(C(=O)O)c3)cc2S(=O)(=O)N1C] | [M + H]⁺ 592.245 | ¹H NMR (400 MHz, DMSO-d₆) δ 13.31 (s, 1H), 7.95 (dd, J = 7.1, 2.5 Hz, 1H), 7.84 (s, 1H), 7.80 - 7.71 (m, 1H), 7.56 (s, 1H), 7.38 (dd, J = 10.7, 8.5 Hz, 1H), 7.19 (t, J = 7.9 Hz, 2H), 7.15 - 7.05 (m, 2H), 7.05 - 6.95 (m, 2H), 6.82 - 6.73 (m, 3H), 6.54 (d, J = 2.3 Hz, 1H), 4.01 (d, J = 16.2 Hz, 1H), 3.78 (s, 1H), 3.33 (s, 1H), 2.54 (s, 3H), 1.57 (dd, J = 14.5, 7.8 Hz, 2H), 1.35 (s, 4H), 0.90 (t, J = 6.7 Hz, 3H). |
| 523 | 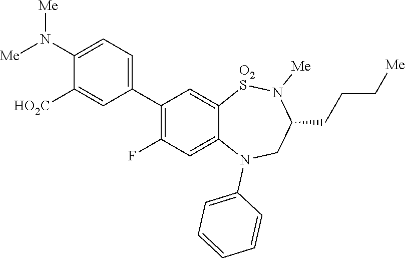 [SMILES: CCCC[C@@H]1CN(c2ccccc2)c2cc(F)c(-c3ccc(N(C)C)c(C(=O)O)c3)cc2S(=O)(=O)N1C] | [M + H]⁺ 526.348 | ¹H NMR (400 MHz, DMSO-d₆) δ 7.99 (s, 1H), 7.91 (d, J = 8.6 Hz, 1H), 7.79 (d, J = 8.6 Hz, 1H), 7.58 (d, J = 8.7 Hz, 1H), 7.27 (dd, J = 8.6, 7.1 Hz, 2H), 7.21 (d, J = 11.8 Hz, 1H), 6.95 - 6.87 (m, 3H), 4.12 (d, J = 16.0 Hz, 1H), 3.77 (s, 1H), 3.52 (s, 1H), 2.89 (s, 6H), 2.62 (s, 3H), 1.61 - 1.55 (m, 2H), 1.45 - 1.29 (m, 4H), 0.91 (t, J = 6.9 Hz, 3H). |
| 524 | 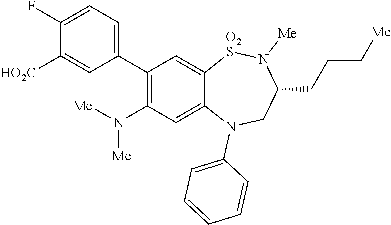 [SMILES: CCCC[C@@H]1CN(c2ccccc2)c2cc(N(C)C)c(-c3ccc(F)c(C(=O)O)c3)cc2S(=O)(=O)N1C] | [M + H]⁺ 526.317 | |
| 525 | 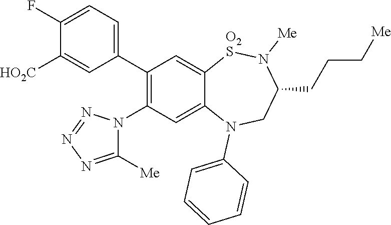 [SMILES: CCCC[C@@H]1CN(c2ccccc2)c2cc(-n3nnnc3C)c(-c3ccc(F)c(C(=O)O)c3)cc2S(=O)(=O)N1C] | [M + H]⁺ 565.402 | ¹H NMR (400 MHz, DMSO-d₆) δ 8.00 (s, 1H), 7.67 (s, 1H), 7.58 - 7.52 (m, 1H), 7.29 (t, J = 7.9 Hz, 2H), 7.26 - 7.14 (m, 2H), 7.03 (d, J = 8.1 Hz, 2H), 6.93 (t, J = 7.3 Hz, 1H), 4.23 (d, J = 15.6 Hz, 1H), 3.77 - 3.64 (m, 2H), 3.66 (s, 1H), 2.74 (s, 3H), 2.25 (s, 3H), 1.65 - 1.59 (m, 2H), 1.42 - 1.31 (m, 4H), 0.91 (t, J = 6.9 Hz, 3H). |
| 526 | 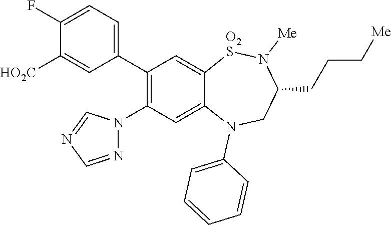 [SMILES: CCCC[C@@H]1CN(c2ccccc2)c2cc(-n3cncn3)c(-c3ccc(F)c(C(=O)O)c3)cc2S(=O)(=O)N1C] | [M + H]⁺ 550.334 | ¹H NMR (400 MHz, DMSO-d₆) δ 13.37 (s, 1H), 8.49 (s, 1H), 8.09 (s, 1H), 7.94 (s, 1H), 7.61 (dd, J = 7.0, 2.4 Hz, 1H), 7.45 (s, 1H), 7.37 - 7.32 (m, 1H), 7.29 (t, J = 8.2, 7.8 Hz, 3H), 7.00 (d, J = 8.1 Hz, 2H), 6.93 (t, J = 7.3 Hz, 1H), 4.19 (d, J = 16.0 Hz, 1H), 3.80 - 3.73 (m, 1H), 3.65 - 3.54 (m, 1H), 2.70 (s, 3H), 1.64 - 1.57 (m, 2H), 1.42 - 1.30 (m, 4H), 0.91 (t, J = 7.0 Hz, 3H). |
| 527 | 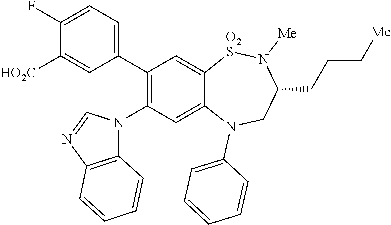 [SMILES: CCCC[C@@H]1CN(c2ccccc2)c2cc(-n3cnc4ccccc43)c(-c3ccc(F)c(C(=O)O)c3)cc2S(=O)(=O)N1C] | [M + H]⁺ 599.239 | ¹H NMR (400 MHz, DMSO-d₆) δ 13.29 (s, 1H), 8.09 (s, 1H), 7.99 (s, 1H), 7.67 (dd, J = 6.9, 2.5 Hz, 1H), 7.65 - 7.61 (m, 1H), 7.42 (s, 1H), 7.30 (t, J = 7.9 Hz, 2H), 7.24 - 7.13 (m, 5H), 7.09 (d, J = 8.1 Hz, 2H), 6.91 (t, J = 7.3 Hz, 1H), 4.22 (d, J = 15.6 Hz, 1H), 3.81 - 3.73 (m, 1H), 3.69 - 3.61 (m, 1H), 2.74 (s, 3H), 1.67 - 1.59 (m, 2H), 1.45 - 1.31 (m, 4H), 0.92 (t, J = 7.0 Hz, 3H). |
| 528 | 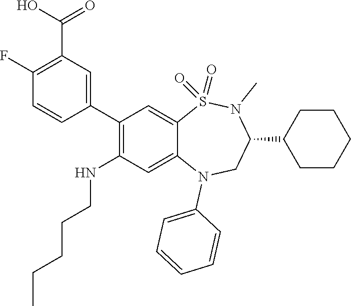 [SMILES: CCCCCNc1cc2c(cc1-c1ccc(F)c(C(=O)O)c1)S(=O)(=O)N(C)[C@H](C1CCCCC1)CN2c1ccccc1] | [M + H]⁺ 594.3 | |
| 529 | 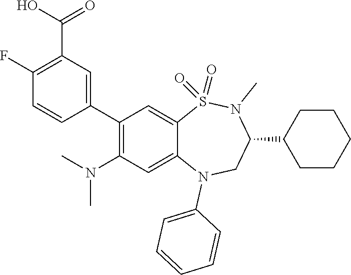 [SMILES: CN(C)c1cc2c(cc1-c1ccc(F)c(C(=O)O)c1)S(=O)(=O)N(C)[C@H](C1CCCCC1)CN2c1ccccc1] | [M + H]⁺ 552.2 | N.A. |
| 530 | 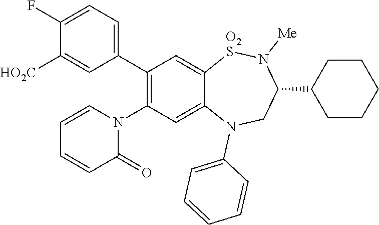 [SMILES: CN1[C@H](C2CCCCC2)CN(c2ccccc2)c2cc(-n3ccccc3=O)c(-c3ccc(F)c(C(=O)O)c3)cc2S1(=O)=O] | [M + H]⁺ 602.5 | |
| 531 | 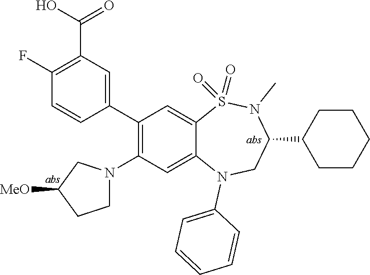 [SMILES: CO[C@@H]1CCN(c2cc3c(cc2-c2ccc(F)c(C(=O)O)c2)S(=O)(=O)N(C)[C@H](C2CCCCC2)CN3c2ccccc2)C1] | [M + H]⁺ 608.3 | |
| 532 | 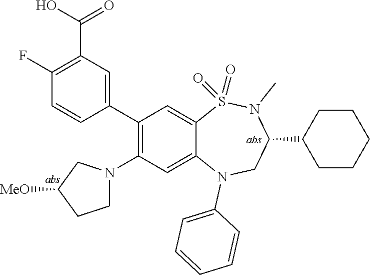 [SMILES: CO[C@H]1CCN(c2cc3c(cc2-c2ccc(F)c(C(=O)O)c2)S(=O)(=O)N(C)[C@H](C2CCCCC2)CN3c2ccccc2)C1] | [M + H]⁺ 608.3 | |
| 533 | 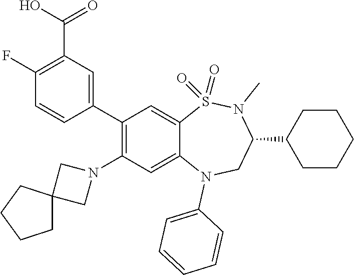 [SMILES: CN1[C@H](C2CCCCC2)CN(c2ccccc2)c2cc(N3CC4(CCCC4)C3)c(-c3ccc(F)c(C(=O)O)c3)cc2S1(=O)=O] | [M + H]⁺ 618.2 | |
| 534 | 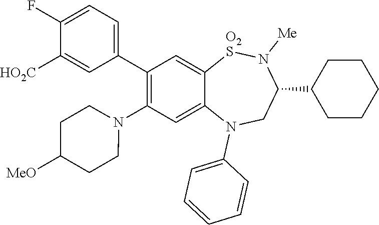 [SMILES: COC1CCN(c2cc3c(cc2-c2ccc(F)c(C(=O)O)c2)S(=O)(=O)N(C)[C@H](C2CCCCC2)CN3c2ccccc2)CC1] | [M + H]⁺ 622.5 | |
| 535 | 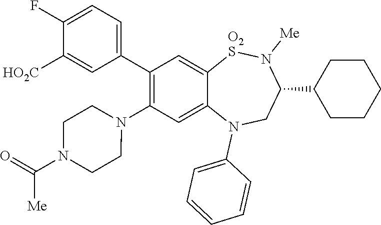 [SMILES: CC(=O)N1CCN(c2cc3c(cc2-c2ccc(F)c(C(=O)O)c2)S(=O)(=O)N(C)[C@H](C2CCCCC2)CN3c2ccccc2)CC1] | [M + H]⁺ 635.6 | |
| 536 | 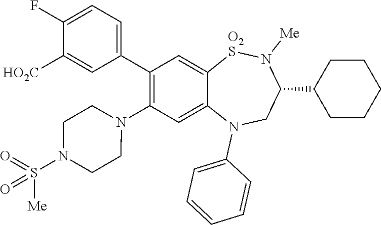 [SMILES: CN1[C@H](C2CCCCC2)CN(c2ccccc2)c2cc(N3CCN(S(C)(=O)=O)CC3)c(-c3ccc(F)c(C(=O)O)c3)cc2S1(=O)=O] | [M + H]⁺ 671.5 | |
| 537 | 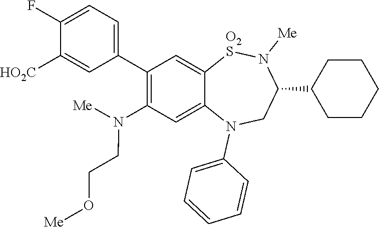 [SMILES: COCCN(C)c1cc2c(cc1-c1ccc(F)c(C(=O)O)c1)S(=O)(=O)N(C)[C@H](C1CCCCC1)CN2c1ccccc1] | [M + H]⁺ 596.5 | |
| 538 | 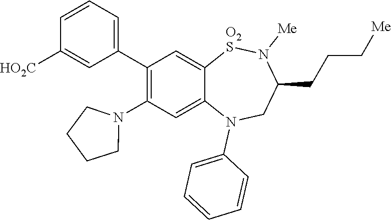 [SMILES: CCCC[C@H]1CN(c2ccccc2)c2cc(N3CCCC3)c(-c3cccc(C(=O)O)c3)cc2S(=O)(=O)N1C] | [M + H]⁺ 534.228 | ¹H NMR (400 MHz, Chloroform-d) δ 8.11 (s, 1H), 8.04 (d, J = 7.8 Hz, 1H), 7.72 (s, 1H), 7.65 (d, J = 7.8 Hz, 1H), 7.49 (t, J = 7.7 Hz, 1H), 7.25 - 7.22 (m, 2H), 6.86 (d, J = 7.8 Hz, 3H), 6.60 (s, 1H), 4.06 - 3.92 (m, 2H), 3.52 - 3.37 (m, 1H), 2.91 - 2.84 (m, 4H), 2.62 (s, 3H), 1.77 - 1.72 (m, 4H), 1.61 (t, J = 9.7 Hz, 1H), 1.49 - 1.38 (m, 5H), 0.93 (t, J = 7.1 Hz, 3H). |
| 539 | 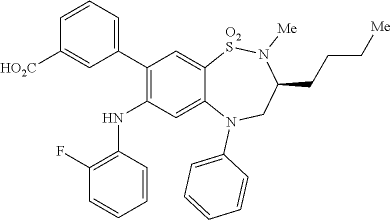 [SMILES: CCCC[C@H]1CN(c2ccccc2)c2cc(Nc3ccccc3F)c(-c3cccc(C(=O)O)c3)cc2S(=O)(=O)N1C] | [M + H]⁺ 574.309 | |
| 540 | 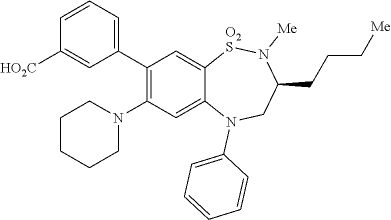 [SMILES: CCCC[C@H]1CN(c2ccccc2)c2cc(N3CCCCC3)c(-c3cccc(C(=O)O)c3)cc2S(=O)(=O)N1C] | [M + H]⁺ 548.339 | |
| 541 | 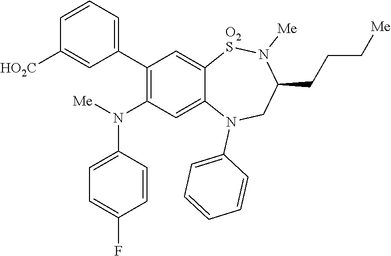 [SMILES: CCCC[C@H]1CN(c2ccccc2)c2cc(N(C)c3ccc(F)cc3)c(-c3cccc(C(=O)O)c3)cc2S(=O)(=O)N1C] | [M + H]⁺ 588.379 | |
| 542 | 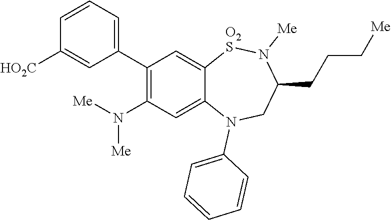 [SMILES: CCCC[C@H]1CN(c2ccccc2)c2cc(N(C)C)c(-c3cccc(C(=O)O)c3)cc2S(=O)(=O)N1C] | [M + H]⁺ 508.275 | |
| 543 | 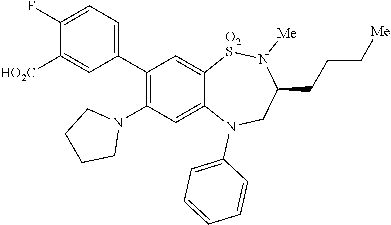 [SMILES: CCCC[C@H]1CN(c2ccccc2)c2cc(N3CCCC3)c(-c3ccc(F)c(C(=O)O)c3)cc2S(=O)(=O)N1C] | [M + H]⁺ 552.200 | ¹H NMR (400 MHz, DMSO-d₆) δ 7.81 (dd, J = 7.0, 2.5 Hz, 1H), 7.68 - 7.63 (m, 1H), 7.45 (s, 1H), 7.37 (dd, J = 10.7, 8.5 Hz, 1H), 7.19 (t, J = 8.1 Hz, 2H), 6.79 - 6.72 (m, 3H), 6.67 (s, 1H), 4.04 (d, J = 16.1 Hz, 1H), 3.91 - 3.78 (m, 1H), 3.49 - 3.16 (m, 1H), 2.97 - 2.76 (m, 4H), 2.47 (s, 3H), 1.78 - 1.68 (m, 4H), 1.63 - 1.46 (m, 2H), 1.35 (s, 4H), 0.91 (t, J = 7.0 Hz, 3H). |
| 544 | 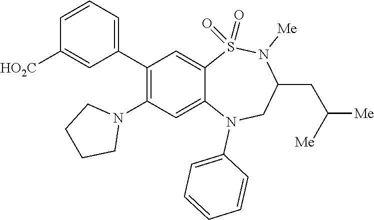 [SMILES: CC(C)CC1CN(c2ccccc2)c2cc(N3CCCC3)c(-c3cccc(C(=O)O)c3)cc2S(=O)(=O)N1C] | [M + H]⁺ 534.299 | ¹H NMR (400 MHz, DMSO-d₆) δ 7.96 - 7.88 (m, 2H), 7.65 (d, J = 7.8 Hz, 1H), 7.57 (t, J = 7.6 Hz, 1H), 7.47 (s, 1H), 7.19 (t, J = 8.1 Hz, 2H), 6.79 - 6.70 (m, 3H), 6.68 (s, 1H), 4.00 (d, J = 15.6 Hz, 2H), 2.94 - 2.78 (m, 4H), 2.46 (s, 3H), 1.73 - 1.68 (m, 5H), 1.55 - 1.47 (m, 1H), 1.41 - 1.29 (m, 1H), 0.94 (d, J = 6.6 Hz, 3H), 0.90 (d, J = 6.5 Hz, 3H). |
| 545 | 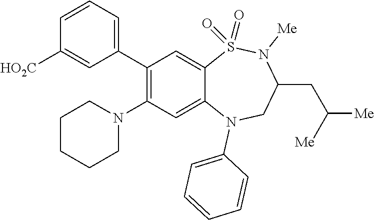 [SMILES: CC(C)CC1CN(c2ccccc2)c2cc(N3CCCCC3)c(-c3cccc(C(=O)O)c3)cc2S(=O)(=O)N1C] | [M + H]⁺ 548.339 | |
| 546 | 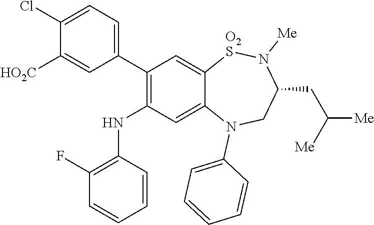 [SMILES: CC(C)C[C@@H]1CN(c2ccccc2)c2cc(Nc3ccccc3F)c(-c3ccc(Cl)c(C(=O)O)c3)cc2S(=O)(=O)N1C] | [M + H]⁺ 608.423 | ¹H NMR (400 MHz, DMSO-d₆) δ 13.43 (s, 1H), 7.90 (s, 2H), 7.67 (d, J = 8.8 Hz, 1H), 7.60 (d, J = 8.3 Hz, 1H), 7.58 (s, 1H), 7.18 (t, J = 8.1 Hz, 2H), 7.15 - 7.08 (m, 2H), 7.05 - 6.99 (m, 2H), 6.80 - 6.74 (m, 3H), 6.52 (d, J = 2.4 Hz, 1H), 4.05 - 3.83 (m, 3H), 2.53 (s, 3H), 1.73 - 1.65 (m, 1H), 1.55 - 1.47 (m, 1H), 1.37 - 1.30 (m, 1H), 0.93 (d, J = 6.6 Hz, 3H), 0.89 (d, J = 6.4 Hz, 3H). |
| 547 | 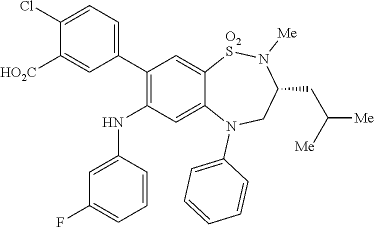 [SMILES: CC(C)C[C@@H]1CN(c2ccccc2)c2cc(Nc3cccc(F)c3)c(-c3ccc(Cl)c(C(=O)O)c3)cc2S(=O)(=O)N1C] | [M + H]⁺ 608.385 | ¹H NMR (400 MHz, DMSO-d₆) δ 13.45 (s, 1H), 8.16 (s, 1H), 7.88 (d, J = 2.2 Hz, 1H), 7.67 - 7.58 (m, 3H), 7.24 (t, J = 8.3 Hz, 2H), 7.14 (q, J = 7.8 Hz, 1H), 7.00 (s, 1H), 6.89 (d, J = 8.1 Hz, 2H), 6.84 - 6.75 (m, 3H), 6.62 (td, J = 8.4, 2.5 Hz, 1H), 4.02 (d, J = 15.9 Hz, 1H), 3.96 - 3.82 (m, 1H), 3.42 - 3.23 (m, 1H), 2.58 (s, 3H), 1.76 - 1.64 (m, 1H), 1.60 - 1.48 (m, 1H), 1.43 - 1.29 (m, 1H), 0.93 (d, J = 6.5 Hz, 3H), 0.90 (d, J = 6.4 Hz, 3H). |
| 548 | 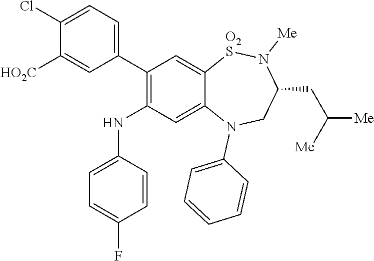 [SMILES: CC(C)C[C@@H]1CN(c2ccccc2)c2cc(Nc3ccc(F)cc3)c(-c3ccc(Cl)c(C(=O)O)c3)cc2S(=O)(=O)N1C] | [M + H]⁺ 608.075 | ¹H NMR (400 MHz, DMSO-d₆) δ 7.89 (s, 1H), 7.86 - 7.74 (m, 1H), 7.64 - 7.51 (m, 3H), 7.21 (t, J = 7.9 Hz, 2H), 7.11 - 7.04 (m, 2H), 7.00 (t, J = 8.8 Hz, 2H), 6.83 - 6.77 (m, 3H), 6.56 (s, 1H), 3.98 (d, J = 16.6 Hz, 1H), 3.92 - 3.87 (m, 1H), 3.36 - 3.31 (m, 1H), 2.54 (s, 3H), 1.72 - 1.67 (m, 1H), 1.55 - 1.50 (m, 1H), 1.38 - 1.28 (m, 1H), 0.93 (d, J = 6.6 Hz, 3H), 0.89 (d, J = 6.5 Hz, 3H). |
| 549 | 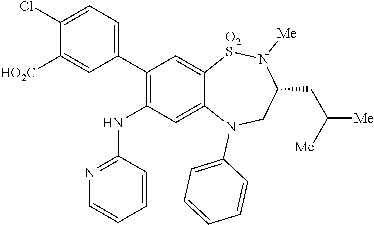 [SMILES: CC(C)C[C@@H]1CN(c2ccccc2)c2cc(Nc3ccccn3)c(-c3ccc(Cl)c(C(=O)O)c3)cc2S(=O)(=O)N1C] | [M + H]⁺ 591.335 | ¹H NMR (400 MHz, DMSO-d₆) δ 8.47 (s, 1H), 7.93 (dd, J = 5.2, 1.9 Hz, 1H), 7.88 (s, 1H), 7.75 (s, 1H), 7.57 (s, 1H), 7.51 (s, 2H), 7.47 - 7.42 (m, 1H), 7.14 (t, J = 8.1 Hz, 2H), 6.80 - 6.73 (m, 3H), 6.73 - 6.66 (m, 2H), 3.98 (d, J = 15.9 Hz, 1H), 3.94 - 3.84 (m, 1H), 3.35 - 3.24 (m, 1H), 2.47 (s, 3H), 1.64 (s, 1H), 1.47 (d, J = 11.1 Hz, 1H), 1.30 (ddd, J = 14.6, 9.5, 5.5 Hz, 1H), 0.88 (d, J = 6.6 Hz, 3H), 0.84 (d, J = 6.4 Hz, 3H). |
| 550 | 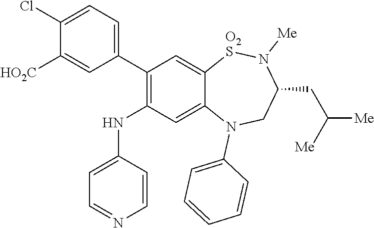 [SMILES: CC(C)C[C@@H]1CN(c2ccccc2)c2cc(Nc3ccncc3)c(-c3ccc(Cl)c(C(=O)O)c3)cc2S(=O)(=O)N1C] | [M + H]⁺ 591.252 | ¹H NMR (400 MHz, DMSO-d₆) δ 8.53 (s, 1H), 8.03 (d, J = 6.6 Hz, 2H), 7.75 (s, 1H), 7.65 (s, 1H), 7.50 (s, 2H), 7.21 (t, J = 8.1, 7.6 Hz, 2H), 7.03 (s, 1H), 6.87 (d, J = 8.1 Hz, 2H), 6.79 (t, J = 7.3 Hz, 1H), 6.70 (d, J = 6.1 Hz, 2H), 4.00 (d, J = 16.1 Hz, 1H), 3.83 - 3.79 (m, 1H), 3.26 (s, 1H), 2.54 (s, 3H), 1.67 - 1.62 (m, 1H), 1.52 - 1.47 (m, 1H), 1.34 - 1.22 (m, 1H), 0.86 (d, J = 6.6 Hz, 3H), 0.83 (d, J = 6.5 Hz, 3H). |
| 551 | 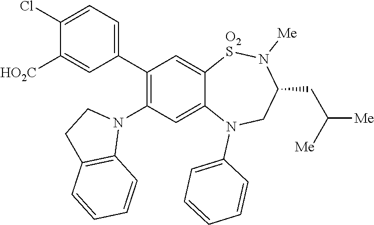 [SMILES: CC(C)C[C@@H]1CN(c2ccccc2)c2cc(N3CCc4ccccc43)c(-c3ccc(Cl)c(C(=O)O)c3)cc2S(=O)(=O)N1C] | [M + H]⁺ 616.255 | ¹H NMR (400 MHz, DMSO-d₆) δ 13.36 (s, 1H), 7.83 (s, 1H), 7.68 (s, 1H), 7.58 (d, J = 8.1 Hz, 1H), 7.47 (d, J = 8.3 Hz, 1H), 7.23 - 7.14 (m, 3H), 7.00 (d, J = 7.4 Hz, 1H), 6.85 (d, J = 8.1 Hz, 2H), 6.80 - 6.72 (m, 2H), 6.58 (t, J = 7.3 Hz, 1H), 6.45 (d, J = 7.9 Hz, 1H), 3.99 (d, J = 16.0 Hz, 1H), 3.88 - 3.75 (m, 1H), 3.43 - 3.29 (m, 2H), 3.30 (s, 01H), 3.27 - 3.25 (m, 1H), 2.95 - 2.74 (m, 2H), 2.54 (s, 3H), 1.66 - 1.62 (m, 1H), 1.52 - 1.45 (m, 1H), 1.34 - 1.22 (m, 1H), 0.86 (d, J = 6.6 Hz, 3H), 0.83 (d, J = 6.5 Hz, 3H). |
| 552 | 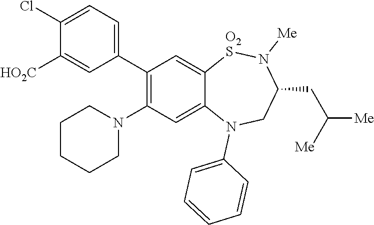 [SMILES: CC(C)C[C@@H]1CN(c2ccccc2)c2cc(N3CCCCC3)c(-c3ccc(Cl)c(C(=O)O)c3)cc2S(=O)(=O)N1C] | [M + H]⁺ 582.344 | ¹H NMR (400 MHz, DMSO-d₆) δ 8.03 (s, 1H), 7.73 (d, J = 8.4 Hz, 1H), 7.55 (d, J = 8.3 Hz, 1H), 7.52 (s, 1H), 7.15 (t, J = 8.0 Hz, 2H), 6.81 - 6.67 (m, 4H), 3.98 (d, J = 15.8 Hz, 1H), 3.85 - 3.81 (m, 1H), 3.35 - 3.22 (m, 1H), 2.71 - 2.60 (m, 4H), 2.45 (s, 3H), 1.63 (s, 1H), 1.49 - 1.42 (m, 1H), 1.37 - 1.30 (m, 6H), 1.31 - 1.24 (m, 1H), 0.86 (d, J = 6.6 Hz, 3H), 0.82 (d, J = 6.4 Hz, 3H). |
| 553 | 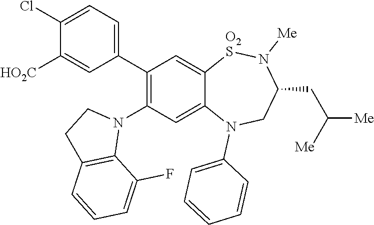 [SMILES: CC(C)C[C@@H]1CN(c2ccccc2)c2cc(N3CCc4cccc(F)c43)c(-c3ccc(Cl)c(C(=O)O)c3)cc2S(=O)(=O)N1C] | [M + H]⁺ 634.264 | ¹H NMR (400 MHz, DMSO-d₆) δ 13.40 (s, 1H), 7.82 (s, 1H), 7.65 (s, 1H), 7.61 (d, J = 8.4 Hz, 1H), 7.51 (d, J = 8.2 Hz, 1H), 7.14 (t, J = 7.7 Hz, 2H), 6.90 (d, J = 6.8 Hz, 1H), 6.82 - 6.68 (m, 6H), 3.98 (d, J = 15.6 Hz, 1H), 3.84 - 3.79 (m, 1H), 3.44 - 3.34 (m, 2H), 3.10 - 2.95 (m, 2H), 2.95 - 2.75 (m, 1H), 2.52 (s, 3H), 1.63 (s, 1H), 1.49 (s, 1H), 1.34 - 1.22 (m, 1H), 0.86 (d, J = 6.6 Hz, 3H), 0.82 (d, J = 6.5 Hz, 3H). |
| 554 | 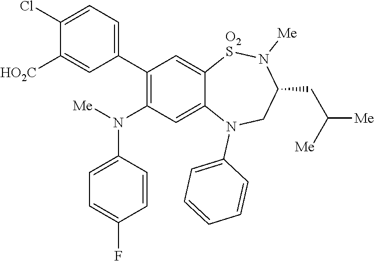 [SMILES: CC(C)C[C@@H]1CN(c2ccccc2)c2cc(N(C)c3ccc(F)cc3)c(-c3ccc(Cl)c(C(=O)O)c3)cc2S(=O)(=O)N1C] | [M + H]⁺ 622.228 | ¹H NMR (400 MHz, DMSO-d₆) δ 13.33 (s, 1H), 7.71 (s, 1H), 7.67 (s, 1H), 7.43 (s, 2H), 7.17 - 7.12 (m, 2H), 6.98 (s, 1H), 6.86 (t, J = 8.8 Hz, 2H), 6.78 (d, J = 8.2 Hz, 2H), 6.75 - 6.67 (m, 3H), 4.02 (d, J = 16.3 Hz, 1H), 3.86 - 3.77 (m, 1H), 3.35 (s, 1H), 3.43 - 3.22 (m, 1H), 2.86 (s, 3H), 2.54 (s, 3H), 1.68 - 1.61 (m, 1H), 1.53 - 1.45 (m, 1H), 1.33 - 1.27 (m, 1H), 0.87 (d, J = 6.6 Hz, 3H), 0.83 (d, J = 6.5 Hz, 3H). |
| 555 | 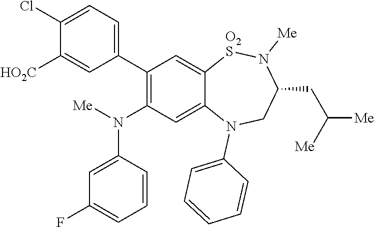 [SMILES: CC(C)C[C@@H]1CN(c2ccccc2)c2cc(N(C)c3cccc(F)c3)c(-c3ccc(Cl)c(C(=O)O)c3)cc2S(=O)(=O)N1C] | [M + H]⁺ 622.082 | ¹H NMR (400 MHz, DMSO-d₆) δ 7.82 (s, 1H), 7.79 (s, 1H), 7.50 (s, 2H), 7.23 - 7.18 (m, 2H), 7.11 (d, J = 6.5 Hz, 2H), 6.89 (d, J = 8.3 Hz, 2H), 6.81 (t, J = 7.4 Hz, 1H), 6.51 (d, J = 9.7 Hz, 3H), 4.12 (d, J = 15.9 Hz, 1H), 3.90 - 3.82 (m, 1H), 3.52 - 3.32 (m, 1H), 2.91 (s, 3H), 2.64 (s, 3H), 1.75 - 1.70 (m, 1H), 1.60 - 1.55 (m, 1H), 1.40 - 1.36 (m, 1H), 0.94 (d, J = 6.6 Hz, 3H), 0.90 (d, J = 6.5 Hz, 3H). |
| 556 | 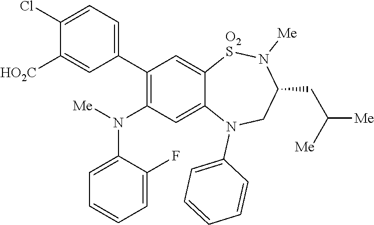 [SMILES: CC(C)C[C@@H]1CN(c2ccccc2)c2cc(N(C)c3ccccc3F)c(-c3ccc(Cl)c(C(=O)O)c3)cc2S(=O)(=O)N1C] | [M + H]⁺ 622.396 | ¹H NMR (400 MHz, DMSO-d₆) δ 7.79 (d, J = 2.2 Hz, 1H), 7.61 (s, 1H), 7.53 (dd, J = 8.3, 2.3 Hz, 1H), 7.46 (d, J = 8.3 Hz, 1H), 7.20 (t, J = 7.9 Hz, 2H), 7.04 - 6.87 (m, 5H), 6.84 - 6.72 (m, 3H), 4.08 (s, 0H), 4.07 (d, J = 16.6 Hz, 1H), 3.97 - 3.81 (m, 1H), 3.57 - 3.45 (m, 1H), 2.96 (s, 3H), 2.56 (s, 3H), 1.79 - 1.65 (m, 1H), 1.62 - 1.52 (m, 1H), 1.42 - 1.32 (m, 1H), 0.94 (d, J = 6.6 Hz, 3H), 0.90 (d, J = 6.5 Hz, 3H). |
| 557 | 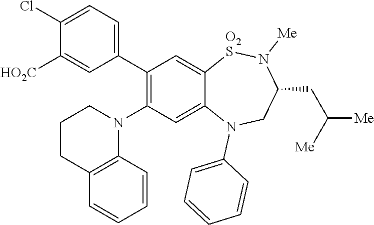 [SMILES: CC(C)C[C@@H]1CN(c2ccccc2)c2cc(N3CCCc4ccccc43)c(-c3ccc(Cl)c(C(=O)O)c3)cc2S(=O)(=O)N1C] | [M + H]⁺ 630.270 | ¹H NMR (400 MHz, DMSO-d₆) δ 13.37 (s, 1H), 7.81 (s, 1H), 7.74 (s, 1H), 7.53 (d, J = 8.3 Hz, 1H), 7.48 (d, J = 8.4 Hz, 1H), 7.14 (t, J = 8.1 Hz, 2H), 7.05 (s, 1H), 6.84 (d, J = 7.4 Hz, 1H), 6.82 - 6.76 (m, 3H), 6.72 (t, J = 7.3 Hz, 1H), 6.53 (t, J = 7.4 Hz, 2H), 4.03 (d, J = 16.1 Hz, 1H), 3.85 - 3.71 (m, 1H), 3.42 - 3.31 (m, 1H), 3.13 - 3.05 (m, 2H), 2.63 - 2.57 (m, 2H), 2.56 (s, 3H), 1.73 - 1.56 (m, 3H), 1.55 - 1.45 (m, 1H), 1.35 - 1.25 (m, 1H), 0.86 (d, J = 6.6 Hz, 3H), 0.83 (d, J = 6.5 Hz, 3H). |

### Ex.558: Synthesis of (R)-2-chloro-5-(3-cyclohexyl-2-methyl-1,1-dioxido-5-phenyl-7-(piperidin-1-yl)-2,3,4,5-tetrahydrobenzo[f][1,2,5]thiadiazepin-8-yl)benzoic acid

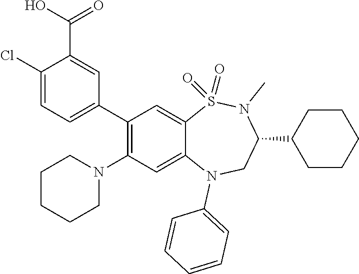 [SMILES: CN1[C@H](C2CCCCC2)CN(c2ccccc2)c2cc(N3CCCCC3)c(-c3ccc(Cl)c(C(=O)O)c3)cc2S1(=O)=O]

Step 1

To a 2-dram glass vial containing a magnetic stir bar and (R)-8-bromo-3-cyclohexyl-7-fluoro-2-methyl-5-phenyl-2,3,4,5-tetrahydrobenzo[f][1,2,5]thiadiazepine 1,1-dioxide (30 mg, 0.064 mmol) were added cesium carbonate (105 mg, 0.321 mmol), DMF (0.4 mL), and piperidine (22 mg, 0.257 mmol). The vial was then sealed and heated to 75° C. After stirring overnight, the reaction mixture was diluted with water and extracted with ethyl acetate (3×). The combined organics were concentrated under reduced pressure and the resulting residue used without further purification as described in Step 2.

Step 2

To a 2-dram glass vial equipped with a magnetic stir bar and containing crude (R)-8-bromo-3-cyclohexyl-2-methyl-5-phenyl-7-(piperidin-1-yl)-2,3,4,5-tetrahydrobenzo[f][1,2,5]thiadiazepine 1,1-dioxide (0.034 g, 0.064 mmol) were added 5-borono-2-chlorobenzoic acid (0.015 g, 0.077 mmol), cesium carbonate (0.063 g, 0.192 mmol), dioxane (0.6 mL) and water (0.1 mL). The resulting mixture was sparged with nitrogen, then treated with PdCl₂(dppf) (7.02 mg, 9.60 μmol). The vial was then flushed with nitrogen, capped and heated to 75° C. After stirring overnight, the reaction mixture was purified by RP-HPLC, affording (R)-2-chloro-5-(3-cyclohexyl-2-methyl-1,1-dioxido-5-phenyl-7-(piperidin-1-yl)-2,3,4,5-tetrahydrobenzo[f][1,2,5]thiadiazepin-8-yl)benzoic acid, Ex.558 (5 mg, 13%) as a colorless solid:

| Example | Structure | MS |
|---|---|---|
| 558 | 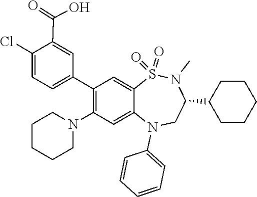 [SMILES: CN1[C@H](C2CCCCC2)CN(c2ccccc2)c2cc(N3CCCCC3)c(-c3ccc(Cl)c(C(=O)O)c3)cc2S1(=O)=O] | [M + H]⁺ 608.3 |

Likewise, the following compound, Ex.559, was prepared by a procedure identical to that described above, except that in Step 1, 4-methoxypiperidine was used in place of piperidine:

| Example | Structure | MS |
|---|---|---|
| 559 | 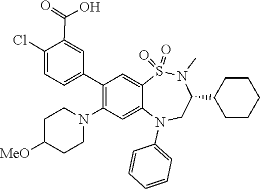 [SMILES: COC1CCN(c2cc3c(cc2-c2ccc(Cl)c(C(=O)O)c2)S(=O)(=O)N(C)[C@H](C2CCCCC2)CN3c2ccccc2)CC1] | [M + H]⁺ 638.3 |

### Ex.560: Synthesis of(R)-5-(3-butyl-7-((2-fluorophenyl)amino)-2-methyl-1,1-dioxido-5-phenyl-2,3,4,5-tetrahydrobenzo[f][1,2,5]thiadiazepin-8-yl)-2,3-difluorobenzoic acid

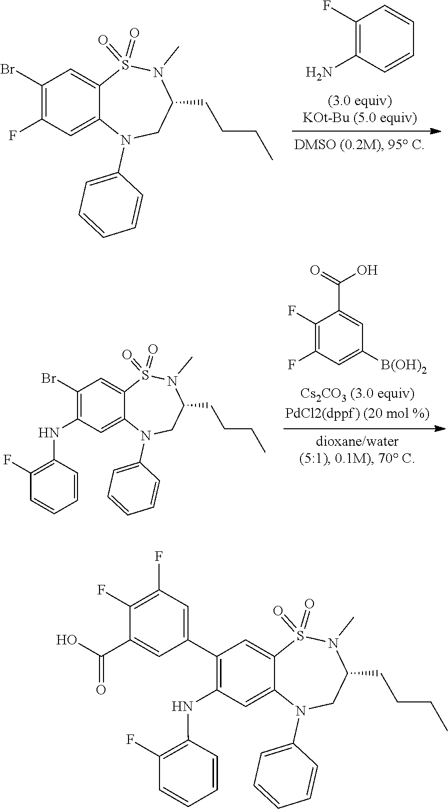 [SMILES: CCCC[C@@H]1CN(c2ccccc2)c2cc(F)c(Br)cc2S(=O)(=O)N1C ; Nc1ccccc1F ; CCCC[C@@H]1CN(c2ccccc2)c2cc(Nc3ccccc3F)c(Br)cc2S(=O)(=O)N1C ; O=C(O)c1cc(B(O)O)cc(F)c1F ; CCCC[C@@H]1CN(c2ccccc2)c2cc(Nc3ccccc3F)c(-c3cc(F)c(F)c(C(=O)O)c3)cc2S(=O)(=O)N1C]

Step 1

To a 2-dram glass vial containing a magnetic stir bar and (R)-8-bromo-3-butyl-7-fluoro-2-methyl-5-phenyl-2,3,4,5-tetrahydrobenzo[f][1,2,5]thiadiazepine 1,1-dioxide (82 mg, 0.186 mmol) were added 2-fluoroaniline (63 mg, 0.557 mmol) and potassium tert-butoxide (104 mg, 0.929 mmol), followed by DMSO (1 mL). The vial was blown out with nitrogen, sealed, and heated to 90° C. After stirring overnight, the reaction mixture was cooled to r.t., partitioned between ethyl acetate and water. The organics were rinsed with water (3×), then concentrated under reduced pressure to reveal (R)-8-bromo-3-butyl-7-((2-fluorophenyl)amino)-2-methyl-5-phenyl-2,3,4,5-tetrahydrobenzo[f][1,2,5]thiadiazepine 1,1-dioxide (60 mg, 91%). ESI-MS m/z=532.1 [M+H]⁺.

Step 2 To a 2-dram vial containing (R)-8-bromo-3-butyl-7-((2-fluorophenyl)amino)-2-methyl-5-phenyl-2,3,4,5-tetrahydrobenzo[f][1,2,5]thiadiazepine 1,1-dioxide (60 mg, 0.170 mmol) and a magnetic stir bar were charged 5-borono-2,3-difluorobenzoic acid (0.034 g, 0.170 mmol), and cesium carbonate (0.110 g, 0.339 mmol), followed by dioxane (0.942 ml) and water (0.188 ml). The resulting mixture was sparged with nitrogen, then treated with PdCl₂(dppf) (0.017 g, 0.023 mmol); the reaction vessel was then sealed and heated to 70° C. After stirring overnight, the reaction mixture was concentrated, then partitioned between water and ethyl acetate. The aqueous phase was extracted with additional ethyl acetate (2×), then the combined organics were concentrated under reduced pressure and purified by RP-HPLC to afford (R)-5-(3-butyl-7-((2-fluorophenyl)amino)-2-methyl-1,1-dioxido-5-phenyl-2,3,4,5-tetrahydrobenzo[f][1,2,5]thiadiazepin-8-yl)-2,3-difluorobenzoic acid, Ex.560 (13 mg, 19%) as a white solid:

| Example | Structure | MS |
|---|---|---|
| 560 | 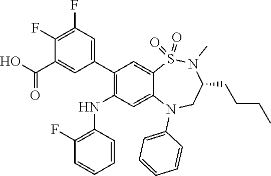 [SMILES: CCCC[C@@H]1CN(c2ccccc2)c2cc(Nc3ccccc3F)c(-c3cc(F)c(F)c(C(=O)O)c3)cc2S(=O)(=O)N1C] | [M + H]⁺ 610.2 |

The following compound, Ex.561 was prepared in a manner identical to that described above, except that in Step 1 (R)-8-bromo-7-fluoro-3-isobutyl-5-isopropyl-2-methyl-2,3,4,5-tetrahydrobenzo[f][1,2,5]thiadiazepine 1,1-dioxide was used in place of (R)-8-bromo-3-butyl-7-fluoro-2-methyl-5-phenyl-2,3,4,5-tetrahydrobenzo[f][1,2,5]thiadiazepine 1,1-dioxide, and in Step 2, 2-fluoro-5-(4,4,5,5-tetramethyl-1,3,2-dioxaborolan-2-yl)benzoic acid was used in place of 5-borono-2,3-difluorobenzoic acid:

| Example | Structure | MS |
|---|---|---|
| 561 | 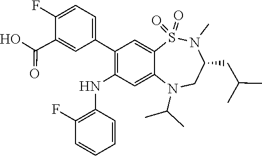 [SMILES: CC(C)C[C@@H]1CN(C(C)C)c2cc(Nc3ccccc3F)c(-c3ccc(F)c(C(=O)O)c3)cc2S(=O)(=O)N1C] | [M + H]⁺ 558.2 |

### Ex.562: Synthesis of(R)-5-(3-cyclohexyl-2-methyl-7-(methylthio)-1,1-dioxido-5-phenyl-2,3,4,5-tetrahydrobenzo[f][1,2,5]thiadiazepin-8-yl)-3-methylthiophene-2-carboxylic acid

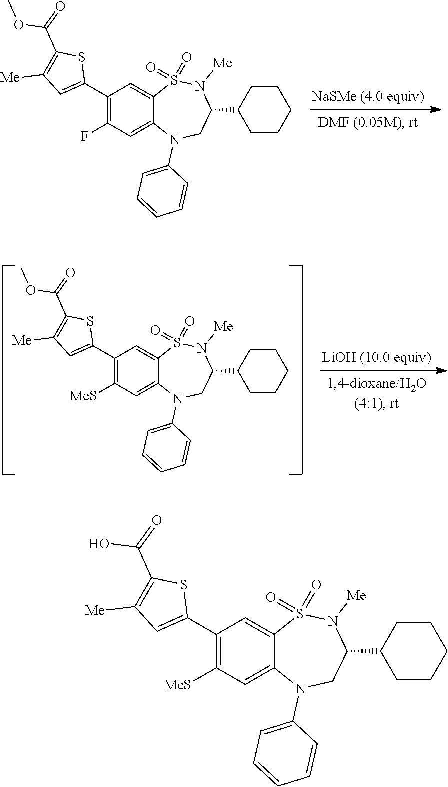

Step 1

In a 4 mL vial equipped with a stir bar, methyl (R)-5-(3-cyclohexyl-7-fluoro-2-methyl-1,1-dioxido-5-phenyl-2,3,4,5-tetrahydrobenzo[f][1,2,5]thiadiazepin-8-yl)-3-methylthiophene-2-carboxylate (20.0 mg, 0.037 mmol, 1.0 equiv) was combined neat with sodium thiomethoxide (10.3 mg, 0.147 mmol, 4.0 equiv). Next, N,N-dimethylformamide (0.74 mL, 0.05M) was added, and the reaction mixture was stirred at room temperature for 6 h. At this time, LCMS analysis indicated full conversion to methyl (R)-5-(3-cyclohexyl-2-methyl-7-(methylthio)-1,1-dioxido-5-phenyl-2,3,4,5-tetrahydrobenzo[f][1,2,5]thiadiazepin-8-yl)-3-methylthiophene-2-carboxylate and the reaction was quenched by the addition of formic acid (0.25 mL). The reaction mixture was concentrated, and the residue was subjected directly to the next step.

Step 2

The residue containing methyl (R)-5-(3-cyclohexyl-2-methyl-7-(methylthio)-1,1-dioxido-5-phenyl-2,3,4,5-tetrahydrobenzo[f][1,2,5]thiadiazepin-8-yl)-3-methylthiophene-2-carboxylate generated above in step 1 was dissolved in a mixture of 1,4-dioxane (1.0 mL) and water (0.25 mL). Next, lithium hydroxide (8.8 mg, 0.369 mmol, 10.0 equiv) was added, and the mixture was stirred at room temperature for 18. The reaction mixture was quenched by the addition of formic acid (0.25 mL), filtered through a 0.45 micron syringe filter using N,N-dimethylformamide to rinse, and purified by RPHPLC. Lyophilization of the isolated product from a mixture of acetonitrile and water afforded (R)-5-(3-cyclohexyl-2-methyl-7-(methylthio)-1,1-dioxido-5-phenyl-2,3,4,5-tetrahydrobenzo[f][1,2,5]thiadiazepin-8-yl)-3-methylthiophene-2-carboxylic acid, Ex.562 (2.64 mg, 12.9% yield). ESI MS M/z=557.2 [M+H]⁺.

| Example | Structure | MS |
|---|---|---|
| 562 | 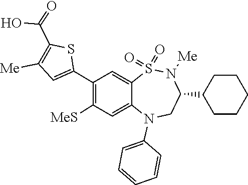 [SMILES: CSc1cc2c(cc1-c1cc(C)c(C(=O)O)s1)S(=O)(=O)N(C)[C@H](C1CCCCC1)CN2c1ccccc1] | [M + H]⁺ 557.2 |

### Ex.563: Synthesis of 5-(3-buty-2-ethyl-1,1-dioxido-5-phenyl-7-(pyrrolidin-1-ylmethyl)-2,3,4,5-tetrahydrobenzo[f][1,2,5]thiadiazepin-8-yl)-2-fluorobenzoic acid

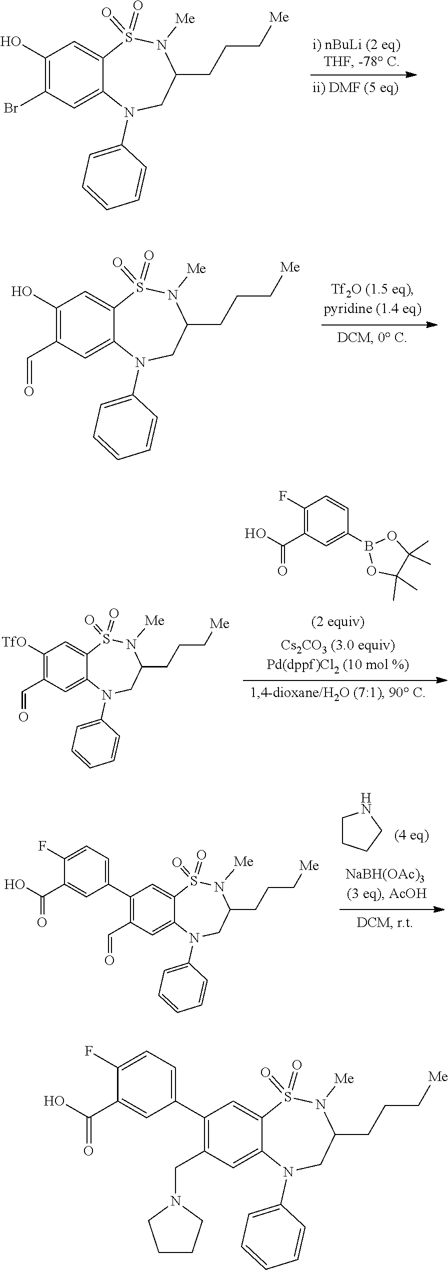

Step 1

To 7-bromo-3-butyl-8-hydroxy-2-methyl-5-phenyl-2,3,4,5-tetrahydrobenzo[f][1,2,5]thiadiazepine 1,1-dioxide (5 g, 11.38 mmol) in THF (37.9 mL) was added nBuLi (16.26 mL, 22.76 mmol) dropwise at −78° C. After stirring for 2 hours at −78° C., DMF (4.41 mL, 56.9 mmol) was added dropwise, and the solution was warmed to room temperature and stirred overnight. The reaction was quenched with saturated aq NH₄Cl and extracted with EtOAc. The combined organic layers were washed with brine, dried over Na₂SO₄, concentrated, then purified by silica gel column chromatography to afford 3-butyl-8-hydroxy-2-methyl-5-phenyl-2,3,4,5-tetrahydrobenzo[f][1,2,5]thiadiazepine-7-carbaldehyde 1,1-dioxide (2 g, 5.15 mmol, 45% yield). ESI MS m/z=389.3 [M+H]⁺.

Step 2

To a vial with 3-butyl-8-hydroxy-2-methyl-5-phenyl-2,3,4,5-tetrahydrobenzo[f][1,2,5]thiadiazepine-7-carbaldehyde 1,1-dioxide (2 g, 5.15 mmol) in dry DCM (25.7 mL), was added pyridine (0.583 mL, 7.21 mmol). The solution was cooled to 0° C., then treated with Tf₂O (1.305 mL, 7.72 mmol) and stirred 0° C. until completion (monitored by LCMS). After 2 hours, the reaction was quenched with saturated NaHCO₃ and extracted with DCM. The combined organic layers were washed with brine, dried over anhydrous Na₂SO₄, concentrated, then purified by silica gel column to afford 3-butyl-7-formyl-2-methyl-1,1-dioxido-5-phenyl-2,3,4,5-tetrahydrobenzo[f][1,2,5]thiadiazepin-8-yl trifluoromethanesulfonate (1.16 g, 2.22 mmol, 43% yield). ¹H NMR (400 MHz, Chloroform-d) δ 10.10 (s, 1H), 7.92 (s, 1H), 7.47 (s, 1H), 7.40 (t, J=8.0 Hz, 2H), 7.18 (t, J=7.4 Hz, 1H), 7.09 (d, J=8.3 Hz, 2H), 4.11-3.98 (m, 2H), 3.74-3.62 (m, 1H), 2.91 (s, 3H), 1.55-1.45 (m, 2H), 1.44-1.28 (m, 4H), 0.94 (t, J=7.1 Hz, 3H). ESI MS m/z=521.2 [M+H]⁺.

Step 3

8-bromo-3-butyl-2-methyl-5-phenyl-2,3,4,5-tetrahydrobenzo[f][1,2,5]thiadiazepine-7-carbaldehyde 1,1-dioxide (300 mg, 0.665 mmol), 2-fluoro-5-(4,4,5,5-tetramethyl-1,3,2-dioxaborolan-2-yl)benzoic acid (354 mg, 1.329 mmol), cesium carbonate (650 mg, 1.994 mmol), and PdCl₂(dppf) (48.6 mg, 0.066 mmol) were combined in vial equipped with a stir bar under nitrogen. Dioxane (5.78 mL) and H₂O (0.867 mL) were added, and the reaction was heated at 90° C. overnight. The reaction mixture was diluted with EtOAc and acidified with 1 N HCl, the aqueous layer was then extracted with EtOAc. The combined organic layers were dried over Na₂SO₄, filtered, concentrated, and purified by silica gel column chromatography to afford 5-(3-butyl-7-formyl-2-methyl-1,1-dioxido-5-phenyl-2,3,4,5-tetrahydrobenzo[f][1,2,5]thiadiazepin-8-yl)-2-fluorobenzoic acid (220 mg, 0.43 mmol, 65% yield). 1H NMR (400 MHz, Chloroform-d) δ 9.90 (s, 1H), 8.06 (dd, J=6.7, 2.5 Hz, 1H), 8.04 (s, 1H), 7.68 (s, 1H), 7.66-7.57 (m, 1H), 7.39-7.30 (m, 3H), 7.08 (t, J=7.5 Hz, 1H), 7.05 (d, J=8.2 Hz, 2H), 4.14 (d, J=15.2 Hz, 1H), 3.93-3.82 (m, 1H), 3.80-3.72 (m, 1H), 2.86 (s, 3H), 2.20 (s, 1H), 1.58-1.45 (m, 2H), 1.43-1.36 (m, 4H), 0.95 (t, J=7.0 Hz, 3H). ESI MS m/z=511.2 [M+H]⁺.

Step 4

To 5-(3-butyl-7-formyl-2-methyl-1,1-dioxido-5-phenyl-2,3,4,5-tetrahydrobenzo[f][1,2,5]thiadiazepin-8-yl)-2-fluorobenzoic acid (0.051 g, 0.1 mmol) in DCM (0.500 mL) was added pyrrolidine (0.017 mL, 0.200 mmol), 1 drop of AcOH, then sodium triacetoxyborohydride (0.032 g, 0.150 mmol) and the reaction was stirred for 2 hours. The reaction mixture was concentrated and the residue was dissolved in a mixture of H₂O and EtOAc. The organic solution was separated, washed with H₂O, dried over Na₂SO₄, filtered, and concentrated. The combined organic layers were dried over Na₂SO₄, filtered, concentrated, and purified by preparative HIPLC to afford 5-(3-butyl-2-methyl-1,1-dioxido-5-phenyl-7-(pyrrolidin-1-ylmethyl)-2,3,4,5-tetrahydrobenzo[f][1,2,5]thiadiazepin-8-yl)-2-fluorobenzoic acid, Ex.563 (6 mg, 0.01 mmol, 11% yield). ¹H NMR (400 MHz, DMSO-d₆) δ 7.94-7.90 (i, 1H), 7.69-7.62 (t, 1H), 7.61 (s, 1H), 7.35 (s, 1H), 7.30 (t, J=9.6 Hz, 1H), 7.15 (t, J=7.9 Hz, 2H), 6.78-6.69 (i, 3H), 4.08 (d, J 16.8 Hz, 1H), 3.75-3.70 (m, 1H), 3.41 (s, 2H), 3.38-3.33 (n, 1H), 2.49 (s, 3H), 2.31-2.25 (m, 4H), 1.59-1.44 (m, 5H), 1.36-1.20 (m, 5H), 0.84 (t, J 6.9 Hz, 3H). ESI MS m/z=566.2 [M+H].

Examples 564 and 565 were made using similar methods to those described above.

| Example | Structure | MS | NMR |
|---|---|---|---|
| 563 | 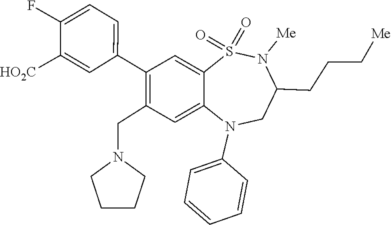 [SMILES: CCCCC1CN(c2ccccc2)c2cc(CN3CCCC3)c(-c3ccc(F)c(C(=O)O)c3)cc2S(=O)(=O)N1C] | [M + H]⁺ 566.2 | ¹H NMR (400 MHz, DMSO-d₆) δ 7.94 − 7.90 (m, 1H), 7.69 − 7.62 (m, 1H), 7.61 (s, 1H), 7.35 (s, 1H), 7.30 (t, J = 9.6 Hz, 1H), 7.15 (t, J = 7.9 Hz, 2H), 6.78 − 6.69 (m, 3H), 4.08 (d, J = 16.8 Hz, 1H), 3.75 − 3.70 (m, 1H), 3.41 (s, 2H), 3.38 − 3.33 (m, 1H), 2.49 (s, 3H), 2.31 − 2.25 (m, 4H), 1.59 − 1.44 (m, 5H), 1.36 − 1.20 (m, 5H), 0.84 (t, J = 6.9 Hz, 3H). |
| 564 | 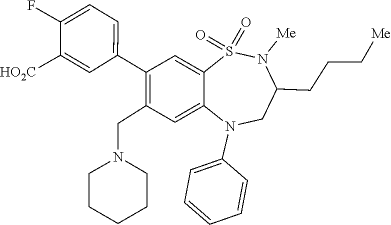 [SMILES: CCCCC1CN(c2ccccc2)c2cc(CN3CCCCC3)c(-c3ccc(F)c(C(=O)O)c3)cc2S(=O)(=O)N1C] | [M + H]⁺ 580.4 | ¹H NMR (400 MHz, DMSO-d₆) δ 7.84 (s, 1H), 7.64 − 7.56 (m, 1H), 7.59 (s, 1H), 7.38 (s, 1H), 7.30 (t, J = 9.4 Hz, 1H), 7.14 (t, J = 7.9 Hz, 2H), 6.74 (d, J = 7.7 Hz, 3H), 4.08 (d, J = 16.4 Hz, 1H), 3.74 − 3.70 (m, 1H), 3.33 − 3.28 (m, 1H), 3.21 − 3.13 (m, 2H), 2.50 (s, 3H), 2.15 − 2.11 (m, 4H), 1.54 − 1.50 (m, 2H), 1.33 − 1.27 (m, 8H), 1.26 − 1.22 (m, 2H), 0.84 (t, J = 6.9 Hz, 3H). |
| 565 | 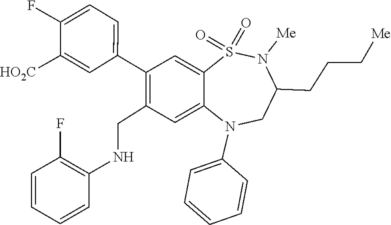 [SMILES: CCCCC1CN(c2ccccc2)c2cc(CNc3ccccc3F)c(-c3ccc(F)c(C(=O)O)c3)cc2S(=O)(=O)N1C] | [M + H]⁺ 606.2 | ¹H NMR (400 MHz, DMSO-d₆) δ 7.90 (d, J = 6.9 Hz, 1H), 7.79 (s, 1H), 7.68 (d, J = 1.0 Hz, 1H), 7.44 (t, J = 9.6 Hz, 1H), 7.39 (s, 1H), 7.01 (t, J = 8.1 Hz, 2H), 6.94 (ddd, J = 12.2, 8.0, 1.3 Hz, 1H), 6.85 (t, J = 7.7 Hz, 1H), 6.70 (t, J = 7.3 Hz, 1H), 6.55 (dd, J = 13.7, 6.9 Hz, 3H), 6.22 (t, J = 8.8 Hz, 1H), 6.16 (s, 1H), 4.33 (dd, J = 16.9, 6.0 Hz, 1H), 4.20 (dd, J = 16.8, 5.9 Hz, 1H), 4.08 (d, J = 16.3 Hz, 1H), 3.79 (s, 1H), 3.41 (s, 1H), 2.53 (s, 3H), 1.67 − 1.47 (m, 2H), 1.36 (d, J = 6.4 Hz, 4H), 0.91 (t, J = 6.8 Hz, 3H). |

### Ex.566: Synthesis of (R)-5-(3-butyl-2,7-dimethyl-1,1-dioxido-5-phenyl-2,3,4,5-tetrahydrobenzo[f][1,2,5]thiadiazepin-8-yl)-2-fluorobenzoic acid

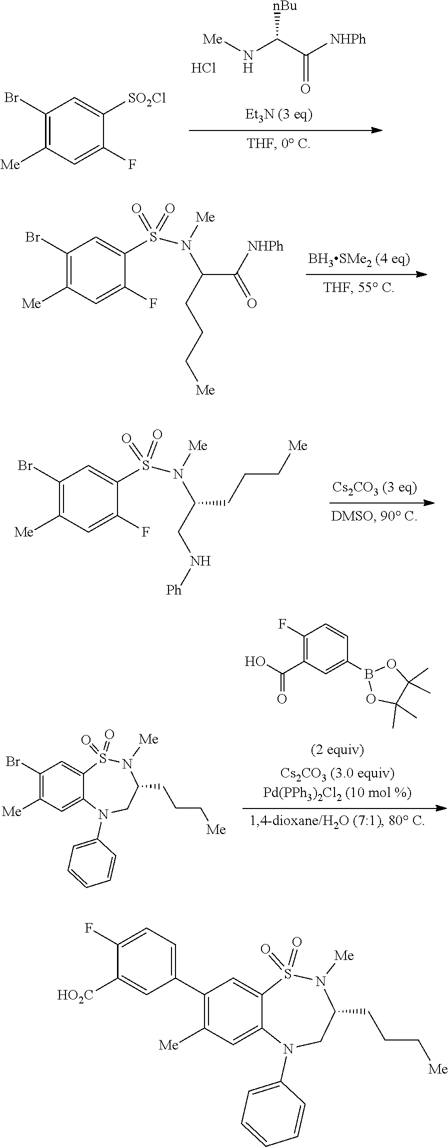

Step 1

To a stirred solution of (R)-2-(methylamino)-N-phenylhexanamide hydrochloride (223 mg, 0.869 mmol) in THE (4.35 mL) was added 5-bromo-2-fluoro-4-methylbenzenesulfonyl chloride (250 mg, 0.869 mmol) and Et₃N (364 μL, 2.61 mmol) at 0° C. and the reaction mixture was then stirred for 16 hours at room temperature. After completion of the reaction (monitored by LCMS), the reaction mixture was filtered through celite, washing with EtOAc. The solution was concentrated and the resulting crude product was purified by silica gel column chromatography (eluent: 0-15% EtOAc/cyclohexane) to afford ((R)-2-((5-bromo-2,4-difluoro-N-methylphenyl)sulfonamido)-N-phenylhexanamide (269 mg, 0.87 mmol, 66% yield) as a white solid. ¹H NMR (400 MHz, Chloroform-d) δ 8.11 (s, 1H), 8.08 (d, J=6.7 Hz, 1H), 7.53 (d, J=7.6 Hz, 2H), 7.35 (t, J=8.5 Hz, 2H), 7.19-7.10 (m, 2H), 4.38 (t, J=7.4 Hz, 1H), 2.97 (d, J=1.5 Hz, 3H), 2.47 (s, 3H), 2.08-1.94 (m, 1H), 1.61-1.47 (m, 1H), 1.33-1.19 (m, 2H), 1.17-1.03 (m, 2H), 0.81 (t, J=7.3 Hz, 3H). ESI MS m/z=471.2 [M+H]⁺.

Step 2

To (R)-2-((5-bromo-2-fluoro-N,4-dimethylphenyl)sulfonamido)-N-phenylhexanamide (269 mg, 0.571 mmol) in THE (1.902 mL) was added BH₃-DMS complex (217 μl, 2.283 mmol). The mixture was then stirred at 55° C. overnight. Upon completion, the reaction was cooled to room temperature, slowly quenched with water, and diluted with EtOAc. The mixture was acidified with 1 N HCl (pH=1) and extracted with EtOAc. The combined organic layers were dried over Na₂SO₄, filtered, concentrated and the resulting crude product was purified by silica gel column chromatography (eluent: 0-20% EtOAc/cyclohexane) to afford ((R)-2-((5-bromo-2,4-difluoro-N-methylphenyl)sulfonamido)-N-phenylhexanamide (192 mg, 0.57 mmol, 74% yield) as an oil. ESI MS m/z=457.2 [M+H]⁺.

Step 3

A mixture of (R)-5-bromo-2-fluoro-N,4-dimethyl-N-(1-(phenylamino)hexan-2-yl)benzenesulfonamide (192 mg, 0.420 mmol) and cesium carbonate (479 mg, 1.469 mmol) in DMSO (1.679 mL) was heated at 90° C. After 1 hour, the reaction was cooled to room temperature and diluted with EtOAc, then washed with H₂O (×5) and brine. The organic layer was dried over Na₂SO₄, filtered, concentrated, and purified by silica gel column chromatography (eluent: 0-10% EtOAc/cyclohexane) to afford (R)-8-bromo-3-butyl-7-fluoro-2-methyl-5-phenyl-2,3,4,5-tetrahydrobenzo[f][1,2,5]thiadiazepine 1,1-dioxide (132 mg, 0.42 mmol, 72% yield). ¹H NMR (400 MHz, Chloroform-d) δ 8.14 (s, 1H), 7.32-7.25 (m, 2H), 7.07 (s, 1H), 6.94 (t, J=7.3 Hz, 1H), 6.85 (d, J=8.1 Hz, 2H), 4.03 (d, J=13.9 Hz, 1H), 3.89-3.85 (m, 1H), 3.50-3.46 (m, 1H), 2.69 (s, 3H), 2.36 (s, 3H), 1.73-1.60 (m, 1H), 1.56-1.45 (m, 1H), 1.45-1.29 (m, 4H), 0.94 (t, J=7.2 Hz, 3H). ESI MS m/z=437.2 [M+H]⁺.

Step 4

(R)-8-bromo-3-butyl-2,7-dimethyl-5-phenyl-2,3,4,5-tetrahydrobenzo[f][1,2,5]thiadiazepine 1,1-dioxide (44 mg, 0.101 mmol), 2-fluoro-5-(4,4,5,5-tetramethyl-1,3,2-dioxaborolan-2-yl)benzoic acid (53.5 mg, 0.201 mmol), cesium carbonate (98 mg, 0.302 mmol), and PdCl₂(dppf) (7.36 mg, 10.06 μmol) were combined in vial equipped with a stir bar under nitrogen. Dioxane (0.875 mL) and Water (0.131 mL) were added, and the reaction was heated at 90° C. overnight. The reaction mixture was diluted with EtOAc and acidified with 1 N HCl, the aqueous layer was then extracted with EtOAc. The combined organic layers were dried over Na₂SO₄, filtered, concentrated, and purified by preparative HPLC to afford (R)-5-(3-butyl-7-fluoro-2-methyl-1,1-dioxido-5-phenyl-2,3,4,5-tetrahydrobenzo[f][1,2,5]thiadiazepin-8-yl)-2-fluorobenzoic acid, Ex.566 (15 mg, 0.10 mmol, 30% yield). ¹H NMR (400 MHz, Chloroform-d) δ 7.83 (dd, J=7.1, 2.4 Hz, 1H), 7.76-7.69 (m, 1H), 7.63 (s, 1H), 7.42 (dd, J=10.6, 8.5 Hz, 1H), 7.32 (s, 1H), 7.20 (t, J=7.8 Hz, 2H), 6.83-6.73 (m, 3H), 4.11 (d, J=16.0 Hz, 1H), 3.87-3.76 (m, 1H), 3.67-3.60 (m, 1H), 2.53 (s, 3H), 2.25 (s, 3H), 1.64-1.51 (m, 2H), 1.42-1.28 (m, 4H), 0.91 (t, J=7.1 Hz, 3H). ESI MS m/z=497.3 [M+H]⁺.

Example 567 was made using a similar method to that described above for Ex.566:

| Example | Structure | MS | NMR |
|---|---|---|---|
| 566 | 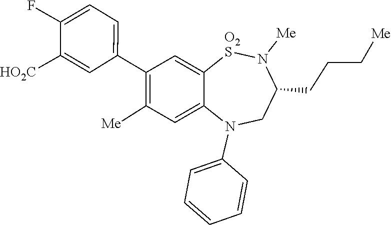 [SMILES: CCCC[C@@H]1CN(c2ccccc2)c2cc(C)c(-c3ccc(F)c(C(=O)O)c3)cc2S(=O)(=O)N1C] | [M + H]⁺ 497.3 | ¹H NMR (400 MHz, Chloroform- d) δ 9.11 (d, J = 2.0 Hz, 1H), 8.90 (d, J = 2.2 Hz, 1H), 8.27 (t, J = 2.1 Hz, 1H), 7.70 (s, 1H), 7.35 (s, 1H), 7.22 (t, J = 8.0 Hz, 2H), 6.85 − 6.77 (m, 3H), 4.13 (d, J = 16.0 Hz, 1H), 3.84 − 3.78 (m, 1H), 3.44 − 3.36 (m, 1H), 2.55 (s, 3H), 2.27 (s, 3H), 1.65 − 1.49 (m, 2H), 1.42 − 1.27 (m, 4H), 0.91 (t, J = 6.9 Hz, 3H). |
| 567 | 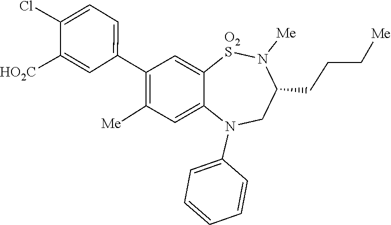 [SMILES: CCCC[C@@H]1CN(c2ccccc2)c2cc(C)c(-c3ccc(Cl)c(C(=O)O)c3)cc2S(=O)(=O)N1C] | [M + H]⁺ 513.2 | ¹H NMR (400 MHz, Chloroform- d) δ 7.78 (d, J = 2.1 Hz, 1H), 7.70 − 7.55 (m, 3H), 7.32 (s, 1H), 7.21 (t, J = 7.8 Hz, 2H), 6.83 − 6.71 (m, 3H), 4.12 (d, J = 16.4 Hz, 1H), 3.85 − 3.75 (m, 1H), 3.55 − 3.51 (m, 1H), 2.53 (s, 3H), 2.26 (s, 3H), 1.64 − 1.49 (m, 2H), 1.41 − 1.28 (m, 4H), 0.90 (t, J = 6.9 Hz, 3H). |

### Ex.568: Synthesis of 5-(3-butyl-2-methyl-1,1-dioxido-5-phenyl-7-(trifluoromethyl)-2,3,4,5-tetrahydrobenzo[f][1,2,5]thiadiazepin-8-yl)-2-fluorobenzoic acid

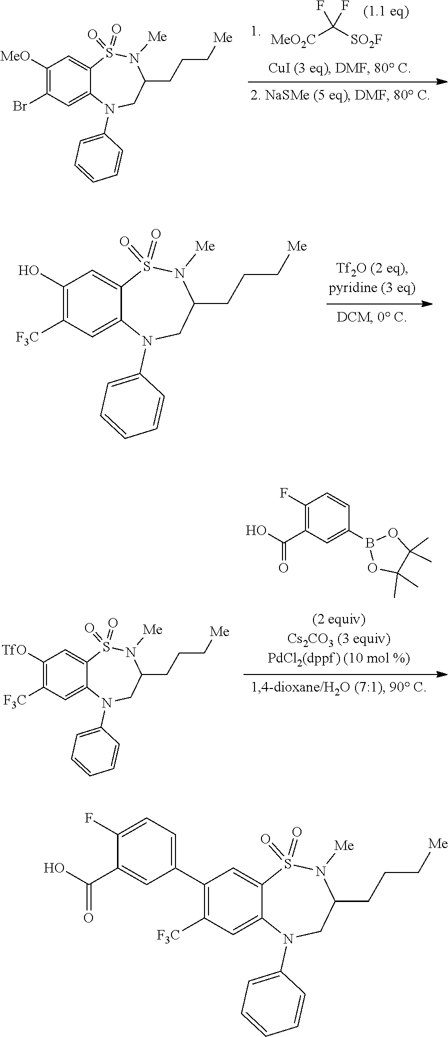 [SMILES: CCCCC1CN(c2ccccc2)c2cc(Br)c(OC)cc2S(=O)(=O)N1C ; COC(=O)C(F)(F)S(=O)(=O)F ; CCCCC1CN(c2ccccc2)c2cc(C(F)(F)F)c(O)cc2S(=O)(=O)N1C ; CC1(C)OB(c2ccc(F)c(C(=O)O)c2)OC1(C)C ; CCCCC1CN(c2ccccc2)c2cc(C(F)(F)F)c(OS(=O)(=O)C(F)(F)F)cc2S(=O)(=O)N1C ; CCCCC1CN(c2ccccc2)c2cc(C(F)(F)F)c(-c3ccc(F)c(C(=O)O)c3)cc2S(=O)(=O)N1C]

Step 1

To a solution of 7-bromo-3-butyl-8-methoxy-2-methyl-5-phenyl-2,3,4,5-tetrahydrobenzo[f][1,2,5]thiadiazepine 1,1-dioxide (1 g, 2.206 mmol) and copper(I) iodide (1.260 g, 6.62 mmol)) in DMF (7.35 mL) at room temperature was added methyl 2,2-difluoro-2-(fluorosulfonyl)acetate (2.246 mL, 17.64 mmol) in DMF (7.35 mL) dropwise over 1 min. The resulting mixture was stirred at 80° C. for 12 h and then cooled down to room temperature and diluted with EtOAc. The organic phase was washed with water and then brine, dried over Na₂SO₄, and concentrated in vacuo to a residue, which was used as a crude mixture. To a stirred solution of 3-butyl-8-methoxy-2-methyl-5-phenyl-7-(trifluoromethyl)-2,3,4,5-tetrahydrobenzo[f][1,2,5]thiadiazepine 1,1-dioxide (0.976 g, 2.206 mmol) in DMF (11.03 mL), sodium thiomethoxide (0.773 g, 11.03 mmol) was added and the reaction mixture was heated for 16 hours at 70° C. Solvent was removed and the resulting crude was purified by silica gel column chromatography (eluent: 0-20% EtOAc/cyclohexane), then preparative HPLC to obtain pure sample of 3-butyl-8-hydroxy-2-methyl-5-phenyl-7-(trifluoromethyl)-2,3,4,5-tetrahydrobenzo[f][1,2,5]thiadiazepine 1,1-dioxide (94 mg, 0.22 mmol, 10% yield). ESI MS m/z=429.2 [M+H]⁺.

Step 2

To a vial with 3-butyl-8-hydroxy-2-methyl-5-phenyl-7-(trifluoromethyl)-2,3,4,5-tetrahydrobenzo[f][1,2,5]thiadiazepine 1,1-dioxide (94 mg, 0.219 mmol) in dry DCM (1.1 mL) was added pyridine (27 μL, 0.33 mmol). The solution was cooled to 0° C., then treated with Tf₂O (74.1 μl, 0.439 mmol)and stirred 0° C. until completion (monitored by LCMS). After 2 hours, the reaction was quenched with saturated NaHCO₃ and extracted with DCM. The combined organic layers were washed with brine, dried over anhydrous Na₂SO₄, concentrated. The crude 3-butyl-2-methyl-1,1-dioxido-5-phenyl-7-(trifluoromethyl)-2,3,4,5-tetrahydrobenzo[f][1,2,5]thiadiazepin-8-yl trifluoromethanesulfonate was used in subsequent steps without further purification. ESI MS m/z=561.1 [M+H]⁺.

Step 3

3-butyl-2-methyl-1,1-dioxido-5-phenyl-7-(trifluoromethyl)-2,3,4,5-tetrahydrobenzo[f][1,2,5]thiadiazepin-8-yl trifluoromethanesulfonate (0.028 g, 0.05 mmol), 2-fluoro-5-(4,4,5,5-tetramethyl-1,3,2-dioxaborolan-2-yl)benzoic acid (0.027 g, 0.100 mmol), Cs₂CO₃ (0.049 g, 0.150 mmol), and PdCl₂(dppf) (3.66 mg, 5.00 μmol) were combined in vial equipped with a stir bar under nitrogen. Dioxane (0.435 mL) and H₂O (0.065 mL) were added, and the reaction was heated at 90° C. overnight. The reaction mixture was diluted with EtOAc and acidified with 1 N HCl, the aqueous layer was then extracted with EtOAc. The combined organic layers were dried over Na₂SO₄, filtered, concentrated, and purified by preparative HPLC to afford 5-(3-butyl-2-methyl-1,1-dioxido-5-phenyl-7-(trifluoromethyl)-2,3,4,5-tetrahydrobenzo[f][1,2,5]thiadiazepin-8-yl)-2-fluorobenzoic acid, Ex.568 (19 mg, 0.04 mmol, 69% yield). ¹H NMR (400 MHz, DMSO-d₆) δ 13.37 (s, 1H), 7.86 (dd, J=6.8, 2.5 Hz, 1H), 7.77 (s, 1H), 7.72-7.64 (m, 1H), 7.56 (s, 1H), 7.44 (dd, J=10.7, 8.5 Hz, 1H), 7.31 (dd, J=8.6, 7.2 Hz, 2H), 7.02-6.92 (m, 3H), 4.19 (d, J=15.8 Hz, 1H), 3.78-3.71 (m, 1H), 3.63-3.59 (m, 1H), 2.68 (s, 3H), 1.65-1.55 (m, 2H), 1.43-1.26 (m, 4H), 0.90 (t, J=7.0 Hz, 3H). ESI MS m/z=551.3 [M+H]⁺.

| Example | Structure | MS | NMR |
|---|---|---|---|
| 568 | 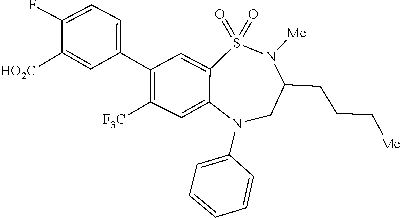 [SMILES: CCCCC1CN(c2ccccc2)c2cc(C(F)(F)F)c(-c3ccc(F)c(C(=O)O)c3)cc2S(=O)(=O)N1C] | [M + H]⁺ 551.3 | ¹H NMR (400 MHz, DMSO-d₆) δ 13.37 (s, 1H), 7.86 (dd, J = 6.8, 2.5 Hz, 1H), 7.77 (s, 1H), 7.72 − 7.64 (m, 1H), 7.56 (s, 1H), 7.44 (dd, J = 10.7, 8.5 Hz, 1H), 7.31 (dd, J = 8.6, 7.2 Hz, 2H), 7.02 − 6.92 (m, 3H), 4.19 (d, J = 15.8 Hz, 1H), 3.78 − 3.71 (m, 1H), 3.63 − 3.59 (m, 1H), 2.68 (s, 3H), 1.65 − 1.55 (m, 2H), 1.43 − 1.26 (m, 4H), 0.90 (t, J = 7.0 Hz, 3H). |

### Ex.569: Synthesis of (R)-5-(3-cyclohexyl-7-(2-fluorophenyl)-2-methyl-1,1-dioxido-2,3,4,5-tetrahydrobenzo[f][1,2,5]thiadiazepin-8-yl)-2-fluorobenzoic acid

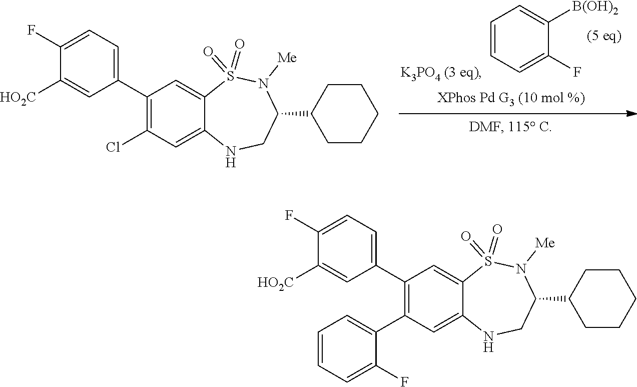 [SMILES: CN1[C@H](C2CCCCC2)CNc2cc(Cl)c(-c3ccc(F)c(C(=O)O)c3)cc2S1(=O)=O ; OB(O)c1ccccc1F ; CN1[C@H](C2CCCCC2)CNc2cc(-c3ccccc3F)c(-c3ccc(F)c(C(=O)O)c3)cc2S1(=O)=O]

To a vial equipped with a stir bar and (R)-5-(7-chloro-3-cyclohexyl-2-methyl-1,1-dioxido-2,3,4,5-tetrahydrobenzo[f][1,2,5]thiadiazepin-8-yl)-2-fluorobenzoic acid (270 mg, 0.578 mmol), (2-fluorophenyl)boronic acid (405 mg, 2.89 mmol), K₃PO₄ (3.0 equiv), and XPhos Pd G3 (48.9 mg, 0.058 mmol) was added DMF (2.9 mL) under N₂. The reaction vial was sealed and the reaction was heated to 115° C. for 1 hour. The reaction mixture was diluted with EtOAc and acidified with 1 N HCl, the aqueous layer was then extracted with EtOAc. The combined organic layers were dried over Na₂SO₄, filtered, and concentrated. The residue was purified by preparative HPLC to afford (R)-5-(3-cyclohexyl-7-(2-fluorophenyl)-2-methyl-1,1-dioxido-2,3,4,5-tetrahydrobenzo[f][1,2,5]thiadiazepin-8-yl)-2-fluorobenzoic acid, Ex.569 (144 mg, 0.273 mmol, 47.3% yield)¹H NMR (400 MHz, DMSO) δ 13.18 (s, 1H), 7.53-7.48 (m, 2H), 7.40-7.33 (m, 1H), 7.30 (td, J=7.6, 1.9 Hz, 1H), 7.24-7.17 (m, 2H), 7.14-7.05 (m, 2H), 6.92-6.85 (m, 2H), 3.91-3.86 (m, 1H), 3.50-3.42 (m, 1H), 3.17-3.13 (m, 1H), 2.87 (s, 3H), 2.10-2.01 (m, 1H), 1.76-1.63 (m, 4H), 1.26-1.11 (m, 4H), 1.09-0.93 (in, 2H). ESI MS m/z=527.2 [M+H]⁺.

Examples 570-572 were made using similar methods to that described above for Ex.569.

| Example | Structure | MS | NMR |
|---|---|---|---|
| 569 | 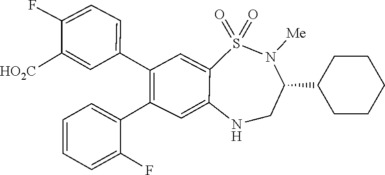 [SMILES: CN1[C@H](C2CCCCC2)CNc2cc(-c3ccccc3F)c(-c3ccc(F)c(C(=O)O)c3)cc2S1(=O)=O] | [M + H]⁺ 527.2 | ¹H NMR (400 MHz, DMSO) δ 13.18 (s, 1H), 7.53 − 7.48 (m, 2H), 7.40 − 7.33 (m, 1H), 7.30 (td, J = 7.6, 1.9 Hz, 1H), 7.24 − 7.17 (m, 2H), 7.14 − 7.05 (m, 2H), 6.92 − 6.85 (m, 2H), 3.91 − 3.86 (m, 1H), 3.50 − 3.42 (m, 1H), 3.17 − 3.13 (m, 1H), 2.87 (s, 3H), 2.10 − 2.01 (m, 1H), 1.76 − 1.63 (m, 4H), 1.26 − 1.11 (m, 4H), 1.09 − 0.93 (m, 2H). |
| 570 | 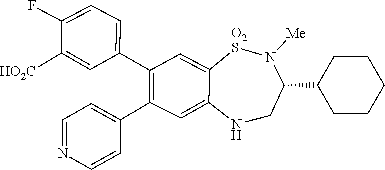 [SMILES: CN1[C@H](C2CCCCC2)CNc2cc(-c3ccncc3)c(-c3ccc(F)c(C(=O)O)c3)cc2S1(=O)=O] | [M + H]⁺ 510.1 | |
| 571 | 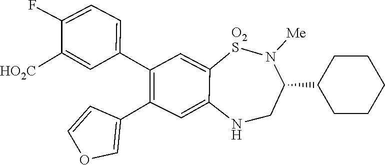 [SMILES: CN1[C@H](C2CCCCC2)CNc2cc(-c3ccoc3)c(-c3ccc(F)c(C(=O)O)c3)cc2S1(=O)=O] | [M + H]⁺ 499.2 | |
| 572 | 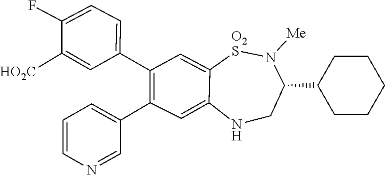 [SMILES: CN1[C@H](C2CCCCC2)CNc2cc(-c3cccnc3)c(-c3ccc(F)c(C(=O)O)c3)cc2S1(=O)=O] | [M + H]⁺ 510.1 | |

### Ex.573: Synthesis of (R)-5-(3-cyclohexyl-7-(4-fluorophenyl)-2-methyl-1,1-dioxido-5-phenyl-2,3,4,5-tetrahydrobenzo[f][1,2,5]thiadiazepin-8-yl)-2-fluorobenzoic acid

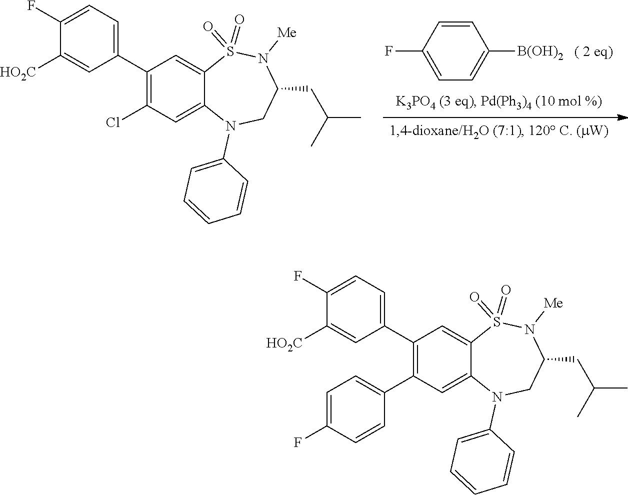 [SMILES: CC(C)C[C@@H]1CN(c2ccccc2)c2cc(Cl)c(-c3ccc(F)c(C(=O)O)c3)cc2S(=O)(=O)N1C ; OB(O)c1ccc(F)cc1 ; CC(C)C[C@@H]1CN(c2ccccc2)c2cc(-c3ccc(F)cc3)c(-c3ccc(F)c(C(=O)O)c3)cc2S(=O)(=O)N1C]

To a microwave vial was added (R)-5-(7-chloro-3-isobutyl-2-methyl-1,1-dioxido-5-phenyl-2,3,4,5-tetrahydrobenzo[f][1,2,5]thiadiazepin-8-yl)-2-fluorobenzoic acid (50 mg, 0.097 mmol), (4-fluorophenyl)boronic acid (27.1 mg, 0.193 mmol), cesium carbonate (95 mg, 0.290 mmol), and Pd(PPh₃)₄ (11.18 mg, 9.67 μmol) in Dioxane (0.774 mL) and water (0.193 mL). The reaction was heated to 120 C using biotage microwave for 90 min. The reaction mixture was diluted with EtOAc and acidified with 1 N HCl, the aqueous layer was then extracted with EtOAc. The combined organic layers were dried over Na₂SO₄, filtered, and concentrated. The residue was purified by preparative HPLC to afford (R)-2-fluoro-5-(7-(4-fluorophenyl)-3-isobutyl-2-methyl-1,1-dioxido-5-phenyl-2,3,4,5-tetrahydrobenzo[f][1,2,5]thiadiazepin-8-yl)benzoic acid, Ex.573 (21 mg, 0.036 mmol, 37.7% yield). ¹H NMR (400 MHz, DMSO) δ 13.30 (s, 1H), 7.84 (s, 1H), 7.68 (dd, J=7.0, 2.5 Hz, 1H), 7.41-7.33 (m, 1H), 7.31 (s, 1H), 7.28-7.20 (m, 3H), 7.19-7.08 (m, 4H), 6.90 (d, J=8.2 Hz, 2H), 6.83 (t, J=7.3 Hz, 1H), 4.14 (d, J=16.2 Hz, 1H), 3.96-3.92 (m, 1H), 3.46-3.42 (m, 1H), 2.62 (s, 3H), 1.72 (d, J=7.0 Hz, 1H), 1.65-1.54 (m, 1H), 1.39 (ddd, J=14.3, 9.3, 5.3 Hz, 1H), 0.93 (dd, J=13.5, 6.5 Hz, 6H).

ESI MS m/z=577.5 [M+H]⁺.

Examples 574-580 were made using similar methods to those described above for Ex.573.

| Example | Structure | MS | NMR |
|---|---|---|---|
| 573 | 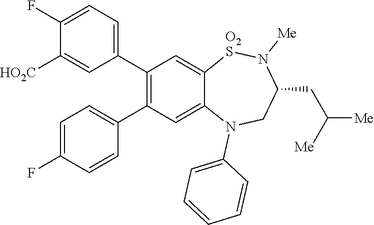 [SMILES: CC(C)C[C@@H]1CN(c2ccccc2)c2cc(-c3ccc(F)cc3)c(-c3ccc(F)c(C(=O)O)c3)cc2S(=O)(=O)N1C] | [M + H]⁺ 577.5 | ¹H NMR (400 MHz, DMSO) δ 13.30 (s, 1H), 7.84 (s, 1H), 7.68 (dd, J = 7.0, 2.5 Hz, 1H), 7.41 - 7.33 (m, 1H), 7.31 (s, 1H), 7.28 - 7.20 (m, 3H), 7.19 - 7.08 (m, 4H), 6.90 (d, J = 8.2 Hz, 2H), 6.83 (t, J = 7.3 Hz, 1H), 4.14 (d, J = 16.2 Hz, 1H), 3.96 - 3.92 (m, 1H), 3.46 - 3.42 (m, 1H), 2.62 (s, 3H), 1.72 (d, J = 7.0 Hz, 1H), 1.65 - 1.54 (m, 1H), 1.39 (ddd, J = 14.3, 9.3, 5.3 Hz, 1H), 0.93 (dd, J = 13.5, 6.5 Hz, 6H). |
| 574 | 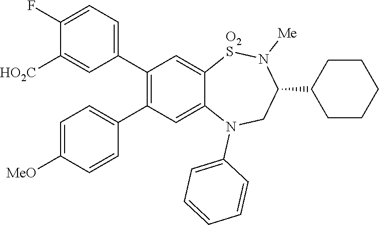 [SMILES: COc1ccc(-c2cc3c(cc2-c2ccc(F)c(C(=O)O)c2)S(=O)(=O)N(C)[C@H](C2CCCCC2)CN3c2ccccc2)cc1] | [M + H]⁺ 589.2 | |
| 575 | 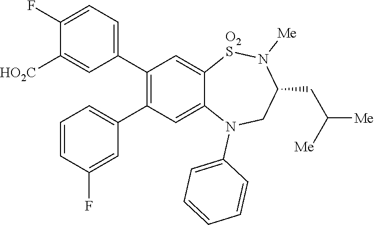 [SMILES: CC(C)C[C@@H]1CN(c2ccccc2)c2cc(-c3cccc(F)c3)c(-c3ccc(F)c(C(=O)O)c3)cc2S(=O)(=O)N1C] | [M + H]⁺ 577.3 | |
| 576 | 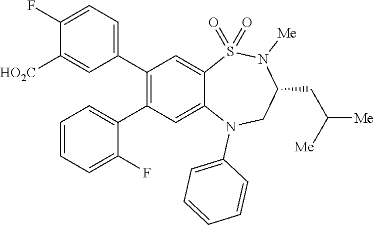 [SMILES: CC(C)C[C@@H]1CN(c2ccccc2)c2cc(-c3ccccc3F)c(-c3ccc(F)c(C(=O)O)c3)cc2S(=O)(=O)N1C] | [M + H]⁺ 577.4 | |
| 577 | 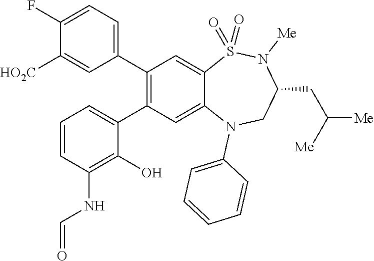 [SMILES: CC(C)C[C@@H]1CN(c2ccccc2)c2cc(-c3cccc(NC=O)c3O)c(-c3ccc(F)c(C(=O)O)c3)cc2S(=O)(=O)N1C] | [M + H]⁺ 618.3 | |
| 578 | 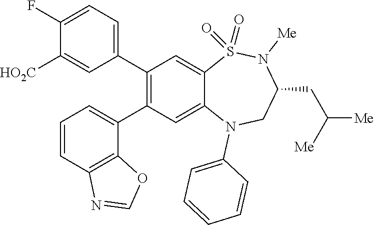 [SMILES: CC(C)C[C@@H]1CN(c2ccccc2)c2cc(-c3cccc4ncoc34)c(-c3ccc(F)c(C(=O)O)c3)cc2S(=O)(=O)N1C] | [M + H]⁺ 600.4 | |
| 579 | 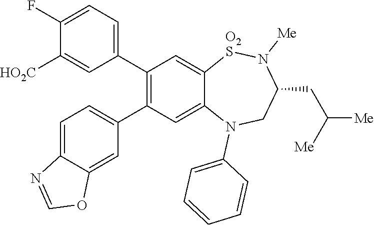 [SMILES: CC(C)C[C@@H]1CN(c2ccccc2)c2cc(-c3ccc4ncoc4c3)c(-c3ccc(F)c(C(=O)O)c3)cc2S(=O)(=O)N1C] | [M + H]⁺ 600.4 | |
| 580 | 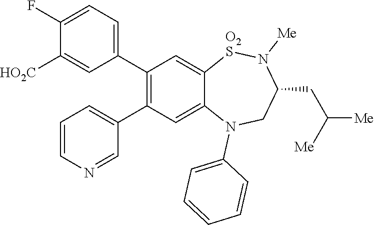 [SMILES: CC(C)C[C@@H]1CN(c2ccccc2)c2cc(-c3cccnc3)c(-c3ccc(F)c(C(=O)O)c3)cc2S(=O)(=O)N1C] | [M + H]⁺ 560.4 | |

The following compound was prepared using a procedure analogous to that described above for Ex.573 starting from (R)-5-(7-chloro-3-cyclohexyl-2,5-dimethyl-1,1-dioxido-2,3,4,5-tetrahydrobenzo[f][1,2,5]thiadiazepin-8-yl)-2-fluorobenzoic acid, Ex.582, the synthesis of which is described below:

| Example | Structure | MS | NMR |
|---|---|---|---|
| 581 | 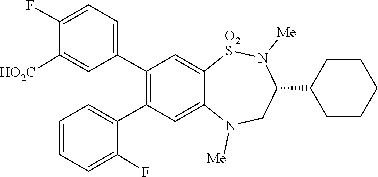 [SMILES: CN1C[C@@H](C2CCCCC2)N(C)S(=O)(=O)c2cc(-c3ccc(F)c(C(=O)O)c3)c(-c3ccccc3F)cc21] | [M + H]⁺ 541.2 | |

### Ex.582: Synthesis of (R)-5-(7-chloro-3-cyclohexyl-2,5-dimethyl-1,1-dioxido-2,3,4,5-tetrahydrobenzo[f][1,2,5]thiadiazepin-8-yl)-2-fluorobenzoic acid

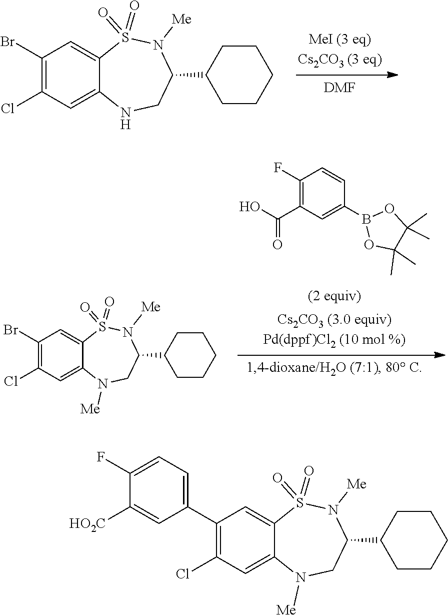

Step 1

To (R)-8-bromo-7-chloro-3-cyclohexyl-2-methyl-2,3,4,5-tetrahydrobenzo[f][1,2,5]thiadiazepine 1,1-dioxide (200 mg, 0.490 mmol) and cesium carbonate (479 mg, 1.471 mmol) in DMF (4.90 mL) was added Mel (92 μl, 1.471 mmol). The reaction was stirred at room temperature overnight. Upon completion, the reaction was diluted with EtOAc, washed with H₂O (x3), then brine. The organic layer was concentrated, then subjected directly to column chromatography (0-10% ethyl acetate/cyclohexane) to afford (R)-8-bromo-7-chloro-3-cyclohexyl-2,5-dimethyl-2,3,4,5-tetrahydrobenzo[f][1,2,5]thiadiazepine 1,1-dioxide (205 mg, 0.490 mmol, >99% yield). ESI MS m/z=421.1 [M+H]⁺.

Step 2

A mixture of (R)-8-bromo-7-chloro-3-cyclohexyl-2,5-dimethyl-2,3,4,5-tetrahydrobenzo[f][1,2,5]thiadiazepine 1,1-dioxide (75 mg, 0.178 mmol), PdCl₂(dppf) (13.01 mg, 0.018 mmol), Cs₂CO₃ (174 mg, 0.533 mmol), and 5-borono-2-fluorobenzoic acid (49.1 mg, 0.267 mmol) were stirred in dioxane (1.42 mL) and water (0.36 mL) at 80° C. for 12 hours. Then the mixture was cooled to r.t., concentrated, and purified by HPLC to afford (R)-5-(7-chloro-3-cyclohexyl-2,5-dimethyl-1,1-dioxido-2,3,4,5-tetrahydrobenzo[f][1,2,5]thiadiazepin-8-yl)-2-fluorobenzoic acid, Ex.582 (56 mg, 0.116 mmol, 65.5% yield). ¹H NMR (400 MHz, DMSO) δ 13.40 (s, 1H), 7.85-7.80 (m, 1H), 7.70-7.65 (m, 1H), 7.55 (d, J=1.3 Hz, 1H), 7.44-7.34 (m, 1H), 7.11 (s, 1H), 4.01 (t, J=13.6 Hz, 1H), 3.45 (dd, J=15.3, 4.2 Hz, 1H), 3.40-3.38 (m, 1H), 3.10 (s, 3H), 2.81 (s, 3H), 2.04 (d, J=13.1 Hz, 1H), 1.78-1.58 (m, 6H), 1.19-1.13 (m, 2H), 1.10-0.96 (m, 2H). ESI MS m/z=481.3 [M+H]⁺.

| Example | Structure | MS | NMR |
|---|---|---|---|
| 582 | 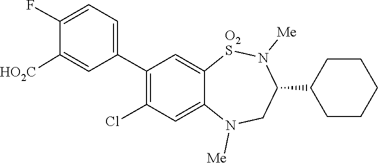 [SMILES: CN1C[C@@H](C2CCCCC2)N(C)S(=O)(=O)c2cc(-c3ccc(F)c(C(=O)O)c3)c(Cl)cc21] | [M + H]⁺ 481.3 | ¹H NMR (400 MHz, DMSO) δ 13.40 (s, 1H), 7.85 − 7.80 (m, 1H), 7.70 − 7.65 (m, 1H), 7.55 (d, J = 1.3 Hz, 1H), 7.44 − 7.34 (m, 1H), 7.11 (s, 1H), 4.01 (t, J = 13.6 Hz, 1H), 3.45 (dd, J = 15.3, 4.2 Hz, 1H), 3.40 − 3.38 (m, 1H), 3.10 (s, 3H), 2.81 (s, 3H), 2.04 (d, J = 13.1 Hz, 1H), 1.78 − 1.58 (m, 6H), 1.19 − 1.13 (m, 2H), 1.10 − 0.96 (m, 2H). |

### Ex.583: Synthesis of (R)-5-(3-cyclohexyl-7-cyclopropyl-2-methyl-1,1-dioxido-5-phenyl-2,3,4,5-tetrahydrobenzo[f][1,2,5]thiadiazepin-8-yl)-2,3-difluorobenzoic acid

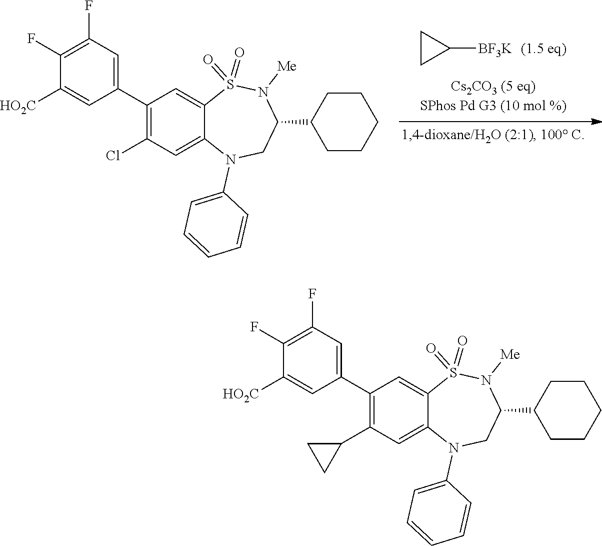

To a vial equipped with a stir bar was added (R)-5-(7-chloro-3-cyclohexyl-2-methyl-1,1-dioxido-5-phenyl-2,3,4,5-tetrahydrobenzo[f][1,2,5]thiadiazepin-8-yl)-2,3-difluorobenzoic acid (80 mg, 0.143 mmol), cyclopropyltrifluoro-14-borane, potassium salt (31.7 mg, 0.214 mmol), SPhos Pd G3 (11.13 mg, 0.014 mmol), and cesium carbonate (232 mg, 0.713 mmol). The vial was sealed and purged with nitrogen, 1,4-Dioxane (1.901 mL) H₂O (0.951 mL) were added, and the reaction was heated to 100° C.

The reaction was concentrated and directly purified by preparative HPLC to afford (R)-5-(3-cyclohexyl-7-cyclopropyl-2-methyl-1,1-dioxido-5-phenyl-2,3,4,5-tetrahydrobenzo[f][1,2,5]thiadiazepin-8-yl)-2,3-difluorobenzoic acid, Ex.583 (30 mg, 0.053 mmol, 37.1% yield). ESI MS m/z=567.2 [M+H]⁺.

| Example | Structure | MS |
|---|---|---|
| 583 | 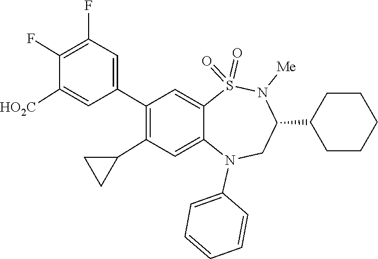 [SMILES: CN1[C@H](C2CCCCC2)CN(c2ccccc2)c2cc(C3CC3)c(-c3cc(F)c(F)c(C(=O)O)c3)cc2S1(=O)=O] | [M + H]⁺ 567.2 |

### Ex.584: Synthesis of (R)-5-(7-(1-(tert-butoxycarbonyl)piperidin-4-yl)-3-cyclohexyl-2-methyl-1,1-dioxido-2,3,4,5-tetrahydrobenzo[f][1,2,5]thiadiazepin-8-yl)-2-fluorobenzoic acid

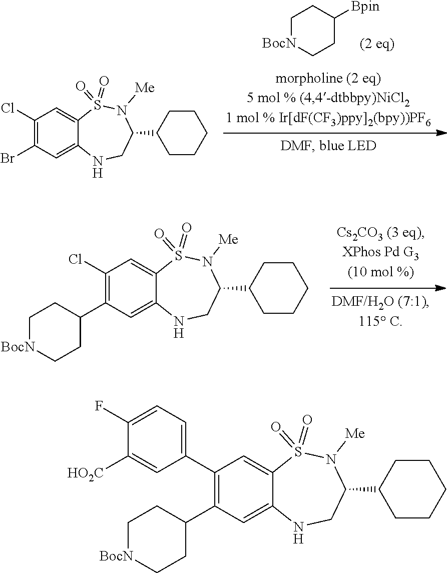

Step 1

A vial was charged with a stir bar, (R)-7-bromo-8-chloro-3-cyclohexyl-2-methyl-2,3,4,5-tetrahydrobenzo[f][1,2,5]thiadiazepine 1,1-dioxide (0.041 g, 0.1 mmol), and tert-butyl 4-(4,4,5,5-tetramethyl-1,3,2-dioxaborolan-2-yl)piperidine-1-carboxylate (0.062 g, 0.20 mmol). A solution of (4,4′-dtbbpy)NiCl₂ (1.990 mg, 5.00 μmol) and Ir[dF(CF₃)ppy]₂ (bpy))PF₆ (1.122 mg, 1.000 μmol) in DMF (1.0 mL) was added, followed by morpholine (0.013 mL, 0.150 mmol). The vial was sealed with parafilm and stirred under 450 nm light source with a fan for 2 h. Upon complete conversion, the reaction mixture was diluted with EtOAc, and the organic layer was washed with 1N HCl, then H₂O (3×5 mL). The combined organic layers were concentrated under N₂ and then subjected to HPLC purification to provide tert-butyl (R)-4-(8-chloro-3-cyclohexyl-2-methyl-1,1-dioxido-2,3,4,5-tetrahydrobenzo[f][1,2,5]thiadiazepin-7-yl)piperidine-1-carboxylate (23 mg, 0.045 mmol, 44.9% yield). ESI MS m/z=456.1 [M-C₄H₈-CO₂+H]⁺.

Step 2

A solution of tert-butyl (R)-4-(8-chloro-3-cyclohexyl-2-methyl-1,1-dioxido-2,3,4,5-tetrahydrobenzo[f][1,2,5]thiadiazepin-7-yl)piperidine-1-carboxylate (23 mg, 0.045 mmol), cesium carbonate (43.9 mg, 0.135 mmol), XPhosPdG3 (3.80 mg, 4.49 μmol), and 2-fluoro-5-(4,4,5,5-tetramethyl-1,3,2-dioxaborolan-2-yl)benzoic acid (17.93 mg, 0.067 mmol) in DMF (0.391 mL) and H₂O (0.059 mL) under nitrogen was heated at 90° C. for 2 hours. Upon complete conversion (monitored by LCMS), the reaction was quenched with 1 M HCl and diluted with EtOAc. The organic layer was washed with H₂O (x3) and brine, then concentrated. The residue was directly subjected to preparative HPLC purification to afford (R)-5-(7-(1-(tert-butoxycarbonyl)piperidin-4-yl)-3-cyclohexyl-2-methyl-1,1-dioxido-2,3,4,5-tetrahydrobenzo[f][1,2,5]thiadiazepin-8-yl)-2-fluorobenzoic acid, Ex.584 (5 mg, 8.12 μmol, 18.08% yield). ESI MS m/z=560.2 [M-C₄H₈-CO₂+H]⁺.

The following compounds were made using similar methods to those described above for Ex.584 starting from (R)-7-bromo-8-chloro-3-cyclohexyl-2-methyl-2,3,4,5-tetrahydrobenzo[f][1,2,5]thiadiazepine 1,1-dioxide or (R)-7-bromo-8-chloro-3-cyclohexyl-2-methyl-5-phenyl-2,3,4,5-tetrahydrobenzo[f][1,2,5]thiadiazepine 1,1-dioxide.

Examples 585-601 were made using methods similar to that described for Example 584.

| Example | Structure | MS | NMR |
|---|---|---|---|
| 584 | 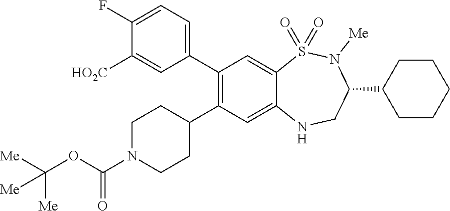 [SMILES: CN1[C@H](C2CCCCC2)CNc2cc(C3CCN(C(=O)OC(C)(C)C)CC3)c(-c3ccc(F)c(C(=O)O)c3)cc2S1(=O)=O] | [M − C₄H₈ − CO₂ + H]⁺ 560.2 | |
| 585 | 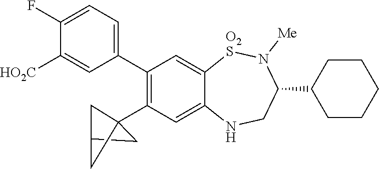 [SMILES: CN1[C@H](C2CCCCC2)CNc2cc(C34CC(C3)C4)c(-c3ccc(F)c(C(=O)O)c3)cc2S1(=O)=O] | [M + H]⁺ 499.1 | |
| 586 | 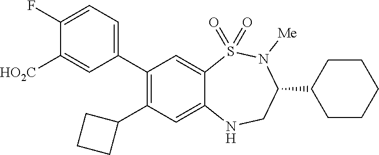 [SMILES: CN1[C@H](C2CCCCC2)CNc2cc(C3CCC3)c(-c3ccc(F)c(C(=O)O)c3)cc2S1(=O)=O] | [M + H]⁺ 487.2 | ¹H NMR (400 MHz, DMSO) δ 13.37 (s, 1H), 7.66 - 7.59 (m, 1H), 7.49 - 7.45 (m, 1H), 7.31 (dd, J = 10.6, 8.4 Hz, 1H), 7.21 (s, 1H), 6.94 (s, 1H), 6.72 - 6.68 (m, 1H), 3.79 (s, 1H), 3.51 (p, J = 8.7 Hz, 1H), 3.20 - 3.15 (m, 1H), 2.79 (s, 3H), 2.06 - 1.88 (m, 5H), 1.88 - 1.74 (m, 1H), 1.74 - 1.62 (m, 7H), 1.21 - 1.16 (m, 3H), 1.00 - 0.95 (m, 2H). |
| 587 | 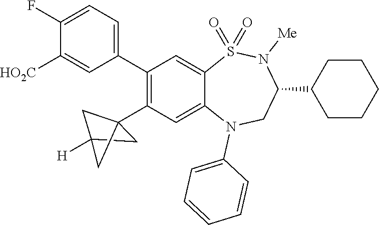 [SMILES: CN1[C@H](C2CCCCC2)CN(c2ccccc2)c2cc(C34CC(C3)C4)c(-c3ccc(F)c(C(=O)O)c3)cc2S1(=O)=O] | [M + H]⁺ 575.3 | |
| 588 | 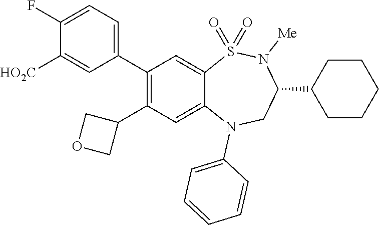 [SMILES: CN1[C@H](C2CCCCC2)CN(c2ccccc2)c2cc(C3COC3)c(-c3ccc(F)c(C(=O)O)c3)cc2S1(=O)=O] | [M + H]⁺ 565.2 | |
| 589 | 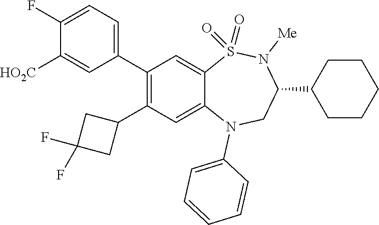 [SMILES: CN1[C@H](C2CCCCC2)CN(c2ccccc2)c2cc(C3CC(F)(F)C3)c(-c3ccc(F)c(C(=O)O)c3)cc2S1(=O)=O] | [M + H]⁺ 599.2 | |
| 590 | 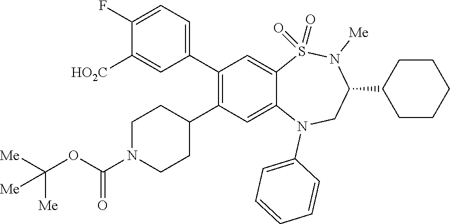 [SMILES: CN1[C@H](C2CCCCC2)CN(c2ccccc2)c2cc(C3CCN(C(=O)OC(C)(C)C)CC3)c(-c3ccc(F)c(C(=O)O)c3)cc2S1(=O)=O] | [M − C₄H₈ − CO₂ + H]⁺ 636.2 | |
| 591 | 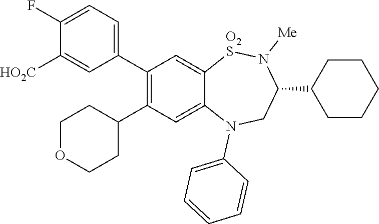 [SMILES: CN1[C@H](C2CCCCC2)CN(c2ccccc2)c2cc(C3CCOCC3)c(-c3ccc(F)c(C(=O)O)c3)cc2S1(=O)=O] | [M + H]⁺ 593.3 | |
| 592 | 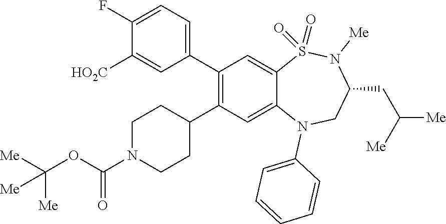 [SMILES: CC(C)C[C@@H]1CN(c2ccccc2)c2cc(C3CCN(C(=O)OC(C)(C)C)CC3)c(-c3ccc(F)c(C(=O)O)c3)cc2S(=O)(=O)N1C] | [M − C₄H₈ − CO₂ + H]⁺ 610.2 | |
| 593 | 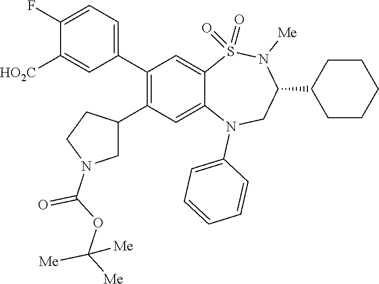 [SMILES: CN1[C@H](C2CCCCC2)CN(c2ccccc2)c2cc(C3CCN(C(=O)OC(C)(C)C)C3)c(-c3ccc(F)c(C(=O)O)c3)cc2S1(=O)=O] | [M − C₄H₈ − CO₂ + H]⁺ 622.2 | |
| 594 | 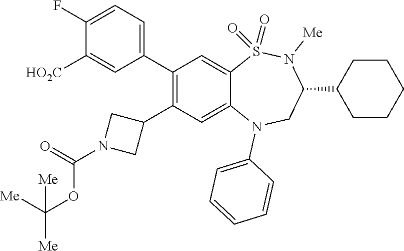 [SMILES: CN1[C@H](C2CCCCC2)CN(c2ccccc2)c2cc(C3CN(C(=O)OC(C)(C)C)C3)c(-c3ccc(F)c(C(=O)O)c3)cc2S1(=O)=O] | [M − C₄H₈ − CO₂ + H]⁺ 608.2 | |
| 595 | 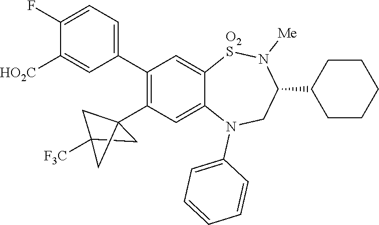 [SMILES: CN1[C@H](C2CCCCC2)CN(c2ccccc2)c2cc(C34CC(C(F)(F)F)(C3)C4)c(-c3ccc(F)c(C(=O)O)c3)cc2S1(=O)=O] | [M + H]⁺ 643.1 | |
| 596 | 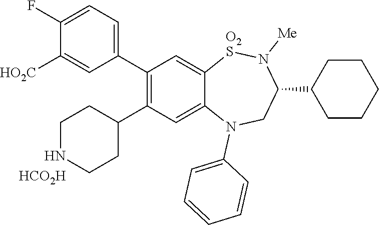 [SMILES: "HCO2H" ; CN1[C@H](C2CCCCC2)CN(c2ccccc2)c2cc(C3CCNCC3)c(-c3ccc(F)c(C(=O)O)c3)cc2S1(=O)=O] | [M + H]⁺ 592.2 | |
| 597 | 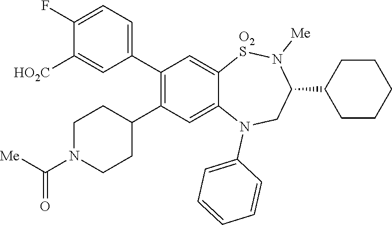 [SMILES: CC(=O)N1CCC(c2cc3c(cc2-c2ccc(F)c(C(=O)O)c2)S(=O)(=O)N(C)[C@H](C2CCCCC2)CN3c2ccccc2)CC1] | [M + H]⁺ 634.3 | |
| 598 | 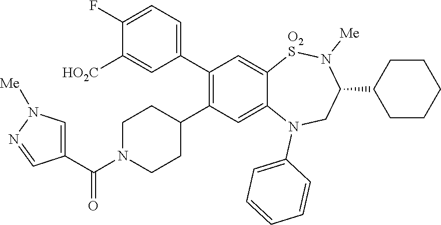 [SMILES: CN1[C@H](C2CCCCC2)CN(c2ccccc2)c2cc(C3CCN(C(=O)c4cnn(C)c4)CC3)c(-c3ccc(F)c(C(=O)O)c3)cc2S1(=O)=O] | [M + H]⁺ 700.3 | |
| 599 | 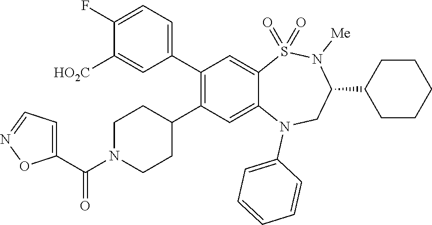 [SMILES: CN1[C@H](C2CCCCC2)CN(c2ccccc2)c2cc(C3CCN(C(=O)c4ccno4)CC3)c(-c3ccc(F)c(C(=O)O)c3)cc2S1(=O)=O] | [M + H]⁺ 687.3 | |
| 600 | 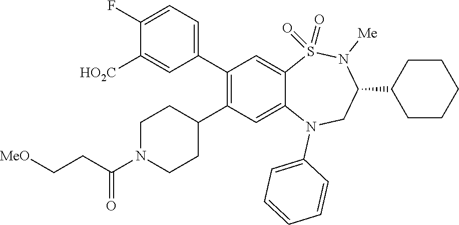 [SMILES: COCCC(=O)N1CCC(c2cc3c(cc2-c2ccc(F)c(C(=O)O)c2)S(=O)(=O)N(C)[C@H](C2CCCCC2)CN3c2ccccc2)CC1] | [M + H]⁺ 678.3 | |
| 601 | 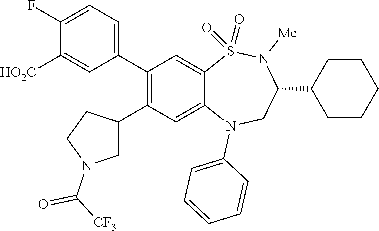 [SMILES: CN1[C@H](C2CCCCC2)CN(c2ccccc2)c2cc(C3CCN(C(=O)C(F)(F)F)C3)c(-c3ccc(F)c(C(=O)O)c3)cc2S1(=O)=O] | [M + H]⁺ 674.3 | |

### Ex.602: Synthesis of (R)-5-(3-cyclohexyl-2-methyl-1,1-dioxido-5-phenyl-7-(thiophen-3-ylethynyl)-2,3,4,5-tetrahydrobenzo[f][1,2,5]thiadiazepin-8-yl)-2-fluorobenzoic acid

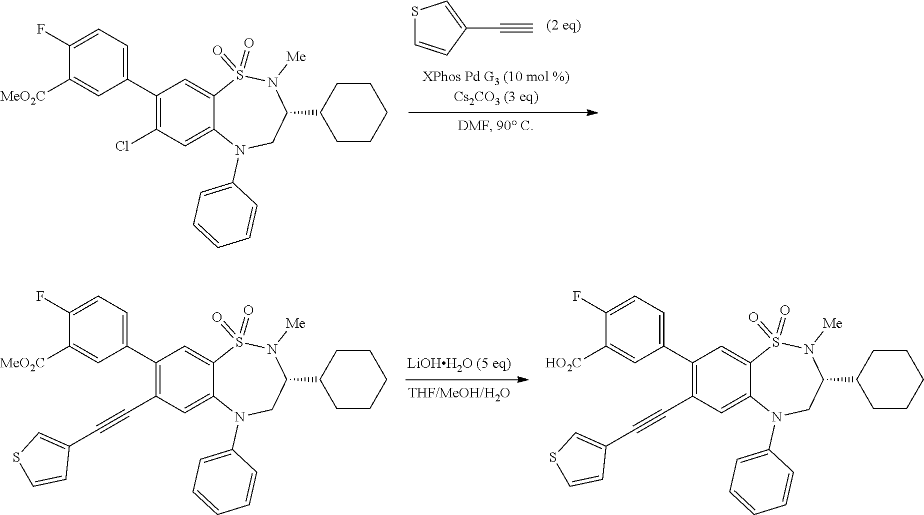

Step 1

A vial containing cesium carbonate (0.195 g, 0.600 mmol), XPhosPdG3 (0.017 g, 0.020 mmol), and methyl (R)-5-(7-chloro-3-cyclohexyl-2-methyl-1,1-dioxido-5-phenyl-2,3,4,5-tetrahydrobenzo[f][1,2,5]thiadiazepin-8-yl)-2-fluorobenzoate (0.11 g, 0.2 mmol) was purged with N₂, then DMF (2.0 mL) was added followed by 3-ethynylthiophene (0.10 mL, 1.000 mmol). The reaction was stirred at 90° C. for 2 hours. Upon complete conversion (monitored by LCMS), the reaction was concentrated and the crude residue was subjected to the subsequent step.

Step 2

To a solution of the crude methyl (R)-5-(3-cyclohexyl-2-methyl-1,1-dioxido-5-phenyl-7-(thiophen-3-ylethynyl)-2,3,4,5-tetrahydrobenzo[f][1,2,5]thiadiazepin-8-yl)-2-fluorobenzoate formed above (0.126 g, 0.2 mmol) in THE (1.000 mL), MeOH (0.500 mL), and H₂O (0.500 mL) was added lithium hydroxide hydrate (0.042 g, 1.000 mmol). The reaction was then stirred at 50° C. for 2 hours. Upon complete conversion (monitored by LCMS), the reaction was quenched with 1 M HCl, and extracted with EtOAc. The combined organic layers were dried over Na₂SO₄, filtered, concentrated, and subjected to preparative HPLC to provide (R)-5-(3-cyclohexyl-2-methyl-1,1-dioxido-5-phenyl-7-(thiophen-3-ylethynyl)-2,3,4,5-tetrahydrobenzo[f][1,2,5]thiadiazepin-8-yl)-2-fluorobenzoic acid, Ex.602 (90 mg, 0.146 mmol, 73.2% yield). ¹H NMR (400 MHz, DMSO) δ 8.12 (dd, J=7.0, 2.5 Hz, 1H), 7.92-7.84 (m, 1H), 7.79 (s, 1H), 7.43 (dd, J=10.6, 8.6 Hz, 1H), 7.30 (t, J=7.8 Hz, 2H), 7.20 (s, 1H), 6.98-6.90 (m, 3H), 5.41-5.36 (m, 1H), 4.37 (d, J=15.8 Hz, 1H), 3.61-3.48 (m, 1H), 2.66 (s, 3H), 1.98-1.87 (m, 2H), 1.72 (s, 2H), 1.62 (d, J=12.7 Hz, 2H), 1.31-1.27 (m, 6H), 1.26-1.11 (m, 3H), 0.98 (t, J=12.7 Hz, 2H). ESI MS m/z=615.1 [M+H]⁺.

Examples 603-605 were made using similar methods to those described above for Ex.602:

| Example | Structure | MS | NMR |
|---|---|---|---|
| 602 | 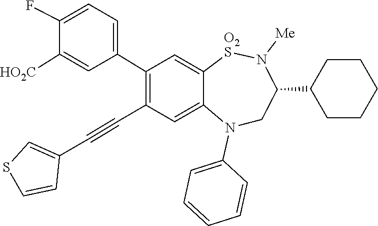 [SMILES: CN1[C@H](C2CCCCC2)CN(c2ccccc2)c2cc(C#Cc3ccsc3)c(-c3ccc(F)c(C(=O)O)c3)cc2S1(=O)=O] | [M + H]⁺ 615.1 | |
| 603 | 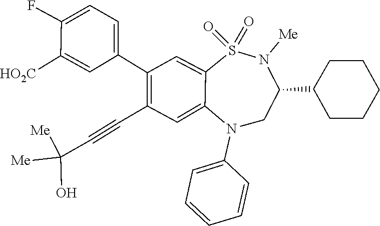 [SMILES: CN1[C@H](C2CCCCC2)CN(c2ccccc2)c2cc(C#CC(C)(C)O)c(-c3ccc(F)c(C(=O)O)c3)cc2S1(=O)=O] | [M − H]⁻ 589.1 | ¹H NMR (400 MHz, DMSO) δ 8.12 (dd, J = 7.0, 2.5 Hz, 1H), 7.92 − 7.84 (m, 1H), 7.79 (s, 1H), 7.43 (dd, J = 10.6, 8.6 Hz, 1H), 7.30 (t, J = 7.8 Hz, 2H), 7.20 (s, 1H), 6.98 − 6.90 (m, 3H), 5.41 − 5.36 (m, 1H), 4.37 (d, J = 15.8 Hz, 1H), 3.61 − 3.48 (m, 1H), 2.66 (s, 3H), 1.98 − 1.87 (m, 2H), 1.72 (s, 2H), 1.62 (d, J = 12.7 Hz, 2H), 1.31 − 1.27 (m, 6H), 1.26 − 1.11 (m, 3H), 0.98 (t, J = 12.7 Hz, 2H). |
| 604 | 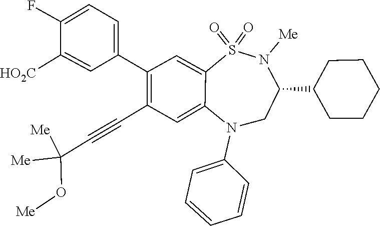 [SMILES: COC(C)(C)C#Cc1cc2c(cc1-c1ccc(F)c(C(=O)O)c1)S(=O)(=O)N(C)[C@H](C1CCCCC1)CN2c1ccccc1] | [M + H]⁺ 605.0 | |
| 605 | 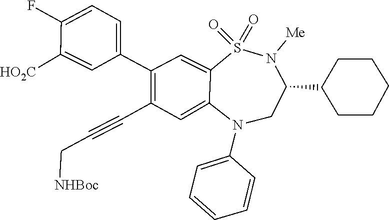 [SMILES: CN1[C@H](C2CCCCC2)CN(c2ccccc2)c2cc(C#CCNC(=O)OC(C)(C)C)c(-c3ccc(F)c(C(=O)O)c3)cc2S1(=O)=O] | [M − C₄H₈ − CO₂ + H]⁺ 606.1 | |

### Ex.606: Synthesis of Rac-5-(7-chloro-3-cyclohexyl-1,1-dioxido-5-phenyl-2,3,4,5-tetrahydrobenzo[b][1,4]thiazepin-8-yl)-2,3-difluorobenzoic acid

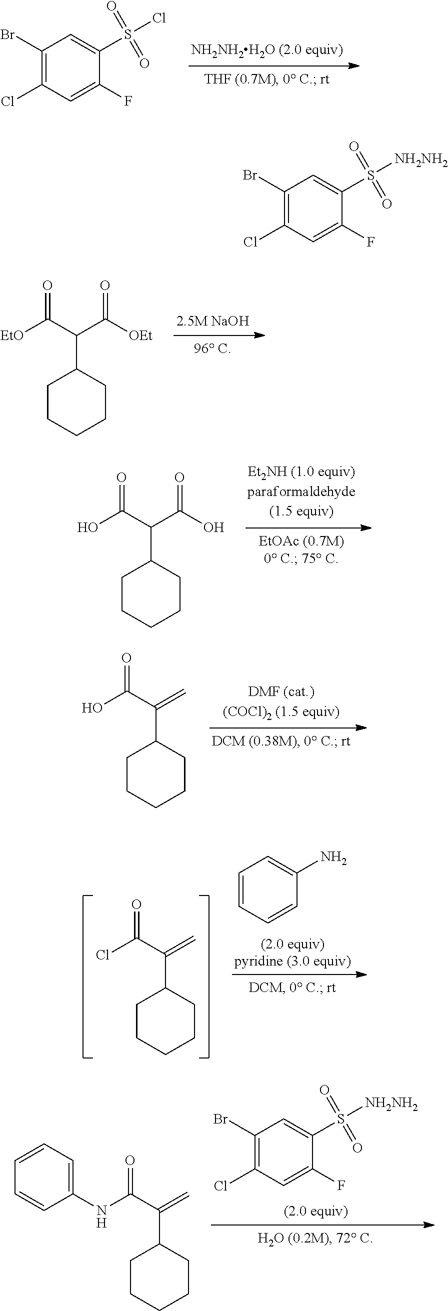

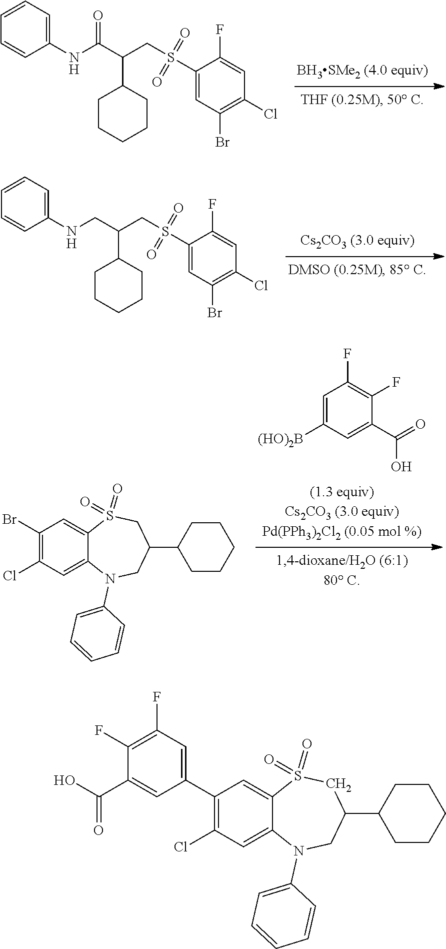 [SMILES: O=C(Nc1ccccc1)C(CS(=O)(=O)c1cc(Br)c(Cl)cc1F)C1CCCCC1 ; O=S(=O)(CC(CNc1ccccc1)C1CCCCC1)c1cc(Br)c(Cl)cc1F ; O=C(O)c1cc(B(O)O)cc(F)c1F ; O=S1(=O)CC(C2CCCCC2)CN(c2ccccc2)c2cc(Cl)c(Br)cc21 ; O=C(O)c1cc(-c2cc3c(cc2Cl)N(c2ccccc2)CC(C2CCCCC2)CS3(=O)=O)cc(F)c1F]

Step 1

In a 40 mL vial equipped with a stir bar, 5-bromo-4-chloro-2-fluorobenzenesulfonyl chloride (900.0 mg, 2.92 mmol, 1.0 equiv) was dissolved in tetrahydrofuran (4.3 mL, 0.68M). The resulting solution was cooled in an ice bath prior to the dropwise addition of hydrazine hydrate (0.57 mL, 5.85 mmol, 2.0 equiv). The reaction mixture was stirred at 0° C. until LCMS analysis indicated full consumption of the starting material. The reaction mixture was then concentrated, and the crude 5-bromo-4-chloro-2-fluoro-benzenesulfonohydrazide was used directly without purification.

Step 2

In a 250 mL round bottom flask equipped with a stir bar and reflux condenser, diethyl 2-cyclohexylmalonate (5.00 g, 20.6 mmol, 1.0 equiv) was suspended in 2.5M aqueous NaOH (75 mL). The reaction mixture was heated using an aluminum bead bath with an external temperature of 96° C. for 4 h. Upon cooling to room temperature, the aqueous phase was washed with cyclohexane and acidified. The aqueous phase was then extracted with ethyl acetate and the combined organic layers were dried over magnesium sulfate, filtered through celite, and concentrated to afford a residue which was used directly without purification.

Step 3

In a 40 mL vial equipped with a stir bar, 2-cyclohexylmalonic acid (907 mg, 4.87 mmol, 1.0 equiv) was suspended in ethyl acetate (6.96 mL, 0.7M). The suspension was cooled in an ice bath, and diethylamine (0.5 mL, 4.87 mmol, 1.0 equiv) was slowly added. Next, paraformaldehyde (219 mg, 7.31 mmol, 1.5 equiv) was added, and the mixture was heated at 75° C. for 3.5 h. Upon cooling to room temperature, the mixture was left overnight. The next morning, it was cooled in an ice bath and water (2.9 mL) was added. The mixture was then acidified with 6M HCl and the aqueous phase was extracted with ethyl acetate. The combined organic layers were dried over magnesium sulfate, filtered through celite, and concentrated to afford the crude residue which was used directly in the next step without purification.

Step 4

In a 250 mL round bottom flask equipped with a stir bar, 2-cyclohexylacrylic acid was dissolved in methylene chloride (19.9 mL) under a nitrogen atmosphere. Next, 3 drops of N,N-dimethylformamide were added, and the mixture was cooled in an ice bath. Oxalyl chloride (0.99 mL, 11.4 mmol, 1.5 equiv) was then added and the ice bath was removed. The mixture was stirred for 2 h and concentrated. The residue was used directly in the next step without purification.

Step 5

In a 100 mL round bottom flask equipped with a stir bar, pyridine (1.8 mL, 22.7, 3.0 equiv) and aniline (1.4 mL, 15.2 mmol, 2.0 equiv) were combined in methylene chloride (19.9 mL). The resulting solution was cooled in an ice bath, and the crude acid chloride generated in step 5 was slowly added as a solution in methylene chloride (5.0 equiv). The reaction was slowly warmed to room temperature and conversion was monitored by LCMS analysis. Upon completion, the mixture was diluted with methylene chloride (100 mL) and the organic layer was washed with 1.2M HCl and brine before being dried over magnesium sulfate. Upon concentration, the crude residue was purified by silica gel column chromatography (gradient elution, 0 to 40% ethyl acetate in cyclohexane) to afford 2-cyclohexyl-N-phenylacrylamide (625.0 mg). ESI MS m/z=230.0 [M+H]⁺.

Step 6

In a 40 mL vial equipped with a stir bar, 2-cyclohexyl-N-phenylacrylamide (625.0 mg, 2.73 mmol, 1.0 equiv) and 5-bromo-4-chloro-2-fluoro-benzenesulfonohydrazide (1.65 g, 5.45 mmol, 2.0 equiv) were combined in water (13.6 mL, 0.2M). The resulting mixture was heated at 72° C. for 22 h. Upon cooling to room temperature, the mixture was diluted with ethyl acetate. The layers were separated and the aqueous phase was extracted with ethyl acetate. The combined organic layers were concentrated and purified by RPHPLC to afford 3-((5-bromo-4-chloro-2-fluorophenyl)sulfonyl)-2-cyclohexyl-N-phenylpropanamide (403.9 mg). ESI MS m/z=502.0 [M+H]⁺.

Step 7

In a 40 mL vial equipped with a stir bar, 3-((5-bromo-4-chloro-2-fluorophenyl)sulfonyl)-2-cyclohexyl-N-phenylpropanamide (403.9 mg, 0.8 mmol, 1.0 equiv) was dissolved in tetrahydrofuran (3.2 mL, 0.25M). Next, borane-dimethyl sulfide complex (0.31 mL, 3.21 mmol, 4.0 equiv) was added, and the mixture was heated at 50° C. for 11 h. At this time, LCMS analysis indicated incomplete conversion of the starting material, and additional borane-dimethyl sulfide complex (0.15 mL) was added. After 9 h, the reaction mixture was cooled to room temperature and carefully quenched with water (1.0 mL) before being concentrated. The residue was passed through a pad of silica gel using ethyl acetate to rinse and concentrated to afford crude N-(3-((5-bromo-4-chloro-2-fluorophenyl)sulfonyl)-2-cyclohexylpropyl)aniline that was used in the next step without purification. ESI MS m/z=488.0 [M+H]⁺.

Step 8

In a 40 mL vial equipped with a stir bar, N-(3-((5-bromo-4-chloro-2-fluorophenyl)sulfonyl)-2-cyclohexylpropyl)aniline (1.0 equiv) was dissolved in dimethyl sulfoxide (3.68 mL, 0.25M). Cesium carbonate (898.4 mg, 2.76 mmol, 3.0 equiv) was added, and the mixture was heated at 85° C. for 19 h. Upon cooling to room temperature, the mixture was concentrated and purified by silica gel column chromatography (gradient elution, 0 to 40% ethyl acetate in cyclohexane) to afford 8-bromo-7-chloro-3-cyclohexyl-5-phenyl-2,3,4,5-tetrahydrobenzo[b][1,4]thiazepine 1,1-dioxide. ESI MS m/z=468.0 [M+H]⁺.

Step 9

In a 4 mL vial equipped with a stir bar, 8-bromo-7-chloro-3-cyclohexyl-5-phenyl-2,3,4,5-tetrahydrobenzo[b][1,4]thiazepine 1,1-dioxide (20.0 mg, 0.04 mmol, 1.0 equiv) was combined neat with 5-borono-2,3-difluorobenzoic acid (11.2 mg, 0.06 mmol, 1.3 equiv), cesium carbonate (41.7 mg, 0.13 mmol, 3.0 equiv), and bis(triphenylphosphine)palladium(II) chloride (1.5 mg, 5 mol %) under a nitrogen atmosphere. Next, 1,4-dioxane (0.43 mL) and water (0.07 mL) were added, and the vial was sealed with electrical tape and heated at 80° C. for 45 min. Upon cooling to room temperature, the reaction mixture was quenched by the addition of formic acid (0.25 mL), passed through a 0.45 micron syringe filter using N,N-dimethylformamide to rinse, and purified by RPHPLC. Lyophilization of the isolated product from acetonitrile and water afforded 5-(7-chloro-3-cyclohexyl-1,1-dioxido-5-phenyl-2,3,4,5-tetrahydrobenzo[b][1,4]thiazepin-8-yl)-2,3-difluorobenzoic acid, Ex.606 (3.69 mg). ESI MS m/z=546.1 [M+H]⁺. ¹H NMR (400 MHz, DMSO-d₆) δ 13.82 (s, 1H), 8.12-7.89 (m, 2H), 7.78 (dt, J=5.9, 1.9 Hz, 1H), 7.54 (s, 1H), 7.35-7.25 (m, 2H), 6.92-6.87 (m, 3H), 4.40 (d, J=15.3 Hz, 1H), 3.61-3.47 (m, 2H), 3.32 (m, 1H), 2.18-2.10 (m, 1H), 1.75-1.58 (m, 6H), 1.29-1.07 (m, 5H).

| Example | Structure | MS | NMR |
|---|---|---|---|
| 606 | 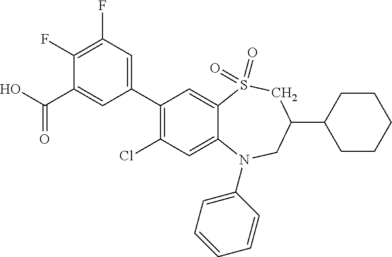 [SMILES: O=C(O)c1cc(-c2cc3c(cc2Cl)N(c2ccccc2)CC(C2CCCCC2)CS3(=O)=O)cc(F)c1F] | [M + H]⁺ 546.1 | ¹H NMR (400 MHz, DMSO-d₆) δ 13.82 (s, 1H), 8.12 - 7.89 (m, 2H), 7.78 (dt, J = 5.9, 1.9 Hz, 1H), 7.54 (s, 1H), 7.35 - 7.25 (m, 2H), 6.92 - 6.87 (m, 3H), 4.40 (d, J = 15.3 Hz, 1H), 3.61 - 3.47 (m, 2H), 3.32 (m, 1H), 2.18 - 2.10 (m, 1H), 1.75 - 1.58 (m, 6H), 1.29 - 1.07 (m, 5H) |

### Ex.607 and Ex.608: Synthesis of cis- and trans-(R)-3-(7-chloro-3-cyclohexyl-2-methyl-1,1-dioxido-5-phenyl-2,3,4,5-tetrahydrobenzo[f][1,2,5]thiadiazepin-8-yl)cyclobutane-1-carboxylic acid

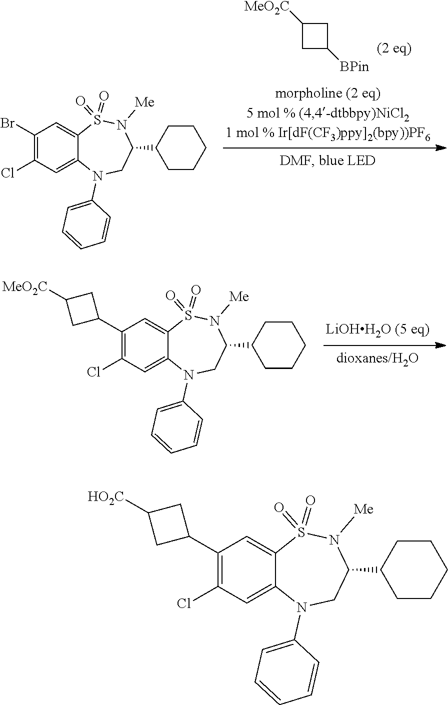

Step 1

A vial was charged with a stir bar, (R)-8-bromo-7-chloro-3-cyclohexyl-2-methyl-5-phenyl-2,3,4,5-tetrahydrobenzo[f][1,2,5]thiadiazepine 1,1-dioxide (0.145 g, 0.3 mmol), methyl 3-(4,4,5,5-tetramethyl-1,3,2-dioxaborolan-2-yl)cyclobutane-1-carboxylate (0.108 g, 0.450 mmol), (4,4′-dtbbpy)NiCl₂ (5.97 mg, 0.015 mmol) and Ir[dF(CF₃)ppy]2 (bpy))PF₆ (3.37 mg, 3.00 μmol). DMF (3.00 ml) was added, followed by morpholine (0.039 ml, 0.450 mmol). The vial was stirred under 450 nm light source with fan for 2 hours. Upon complete conversion, the reaction mixture was concentrated under N₂ and then subjected to column chromatography to provide methyl (R)-3-(7-chloro-3-cyclohexyl-2-methyl-1,1-dioxido-5-phenyl-2,3,4,5-tetrahydrobenzo[f][1,2,5]thiadiazepin-8-yl)cyclobutane-1-carboxylate as a mixture of isomers (122 mg, 0.236 mmol, 79% yield). ESI MS m/z=517.1 [M+H]⁺.

Step 2

To methyl (R)-3-(7-chloro-3-cyclohexyl-2-methyl-1,1-dioxido-5-phenyl-2,3,4,5-tetrahydrobenzo[f][1,2,5]thiadiazepin-8-yl)cyclobutane-1-carboxylate (0.052 g, 0.1 mmol), in Dioxane (0.833 ml) and water (0.167 ml) was added LiOH (2.395 mg, 0.100 mmol). The reaction was stirred at room temperature until complete conversion observed by LCMS and then was quenched with HCl, and extracted with EtOAc. The organic layer was dried over Na₂SO₄, concentrated, and directly subjected to HPLC purification to provide (1S,3s)-3-((R)-7-chloro-3-cyclohexyl-2-methyl-1,1-dioxido-5-phenyl-2,3,4,5-tetrahydrobenzo[f][1,2,5]thiadiazepin-8-yl)cyclobutane-1-carboxylic acid (10 mg, 0.020 mmol, 19.88% yield). ESI MS m/z=503.2 [M+H]⁺.

(1R,3r)-3-((R)-7-chloro-3-cyclohexyl-2-methyl-1,1-dioxido-5-phenyl-2,3,4,5-tetrahydrobenzo[f][1,2,5]thiadiazepin-8-yl)cyclobutane-1-carboxylic acid (10 mg, 0.020 mmol, 19.88% yield). ESI MS m/z=503.2 [M+H]⁺.

| Example | Structure | MS | NMR |
|---|---|---|---|
| 607 | 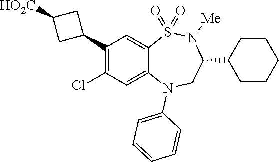 [SMILES: CN1[C@H](C2CCCCC2)CN(c2ccccc2)c2cc(Cl)c([C@H]3C[C@@H](C(=O)O)C3)cc2S1(=O)=O] | [M + H]⁺ 503.2 | |
| 608 | 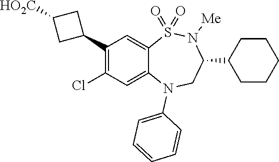 [SMILES: CN1[C@H](C2CCCCC2)CN(c2ccccc2)c2cc(Cl)c([C@H]3C[C@H](C(=O)O)C3)cc2S1(=O)=O] | [M + H]⁺ 503.2 | |

Biological Activity

Methods:

HBV Infection in HepG2-NTCP Cells

HepG2-NTCP A3 cells were maintained in DMEM media supplemented with GlutaMAX™ 10% fetal bovine serum, 1% penicillin/streptomycin, and 5 ug/mL puromycin at 37° C. in a humidified atmosphere with 5% CO₂ in a collagen-coated tissue culture flask.

HepG2-NTCP cells were seeded in 384 well plate containing 16,000 cells/well two days prior to the infection. On the day of infection, compounds were 3-fold serially diluted in DMSO and pre-incubated with HepG2-NTCP cells for two hours before purified HBV addition. HBV infection was carried out at 2000 GE/cell with 4% PEG, and the final concentration of DMSO is 0.5%. On day one post infection, HBV-containing media was aspirated, cells were washed once with PBS and then maintained in 2.5% DMSO containing media for the remainder of the assay.

Supernatants from infected HepG2-NTCP cells were collected at day 8 post infection, and the amount of viral antigen HBeAg was measured by HBeAg AlphaLISA detection kit (PerkinElmer) following the manufacturer's recommended protocol. Irrespective of readout, compound concentrations that reduce viral product accumulation in supernatants by 50% relative to DMSO controls (EC₅₀) are reported. EC₅₀ ranges are as follows: A<0.1 μM; B 0.1-1 μM; C>1 μM.

TABLE 1
Summary of HBV Activities

| Example | Cellular | Example | Cellular |
|---|---|---|---|
| Number | EC₅₀ (μM) | Number | EC₅₀ (μM) |
| 1 | C | 2 | C |
| 3 | C | 4 | B |
| 5 | C | 6 | C |
| 7 | B | 8 | C |
| 9 | B | 10 | C |
| 11 | C | 12 | C |
| 13 | C | 14 | B |
| 15 | C | 16 | B |
| 17 | C | 18 | B |
| 19 | B | 20 | B |
| 21 | B | 22 | C |
| 23 | C | 24 | C |
| 25 | A | 26 | A |
| 27 | B | 28 | B |
| 29 | B | 30 | B |
| 31 | B | 32 | B |
| 33 | B | 34 | A |
| 35 | B | 36 | B |
| 37 | B | 38 | A |
| 39 | B | 40 | C |
| 41 | C | 42 | C |
| 43 | C | 43 | C |
| 45 | C | 46 | B |
| 47 | B | 48 | A |
| 49 | C | 50 | B |
| 51 | A | 52 | B |
| 53 | C | 54 | B |
| 55 | B | 56 | B |
| 57 | B | 58 | B |
| 59 | A | 60 | A |
| 61 | B | 62 | A |
| 63 | A | 64 | B |
| 65 | A | 66 | B |
| 67 | A | 68 | A |
| 69 | C | 70 | C |
| 71 | B | 72 | A |
| 73 | A | 74 | C |
| 75 | B | 76 | B |
| 77 | B | 78 | B |
| 79 | B | 80 | A |
| 81 | A | 82 | C |
| 83 | C | 84 | C |
| 85 | A | 86 | B |
| 87 | B | 88 | C |
| 89 | B | 90 | B |
| 91 | C | 92 | B |
| 93 | B | 94 | B |
| 95 | B | 96 | B |
| 97 | B | 98 | C |
| 99 | C | 100 | C |
| 101 | B | 102 | C |
| 103 | B | 104 | B |
| 105 | B | 106 | C |
| 107 | C | 108 | C |
| 109 | C | 110 | C |
| 111 | C | 112 | C |
| 113 | C | 114 | C |
| 115 | C | 116 | A |
| 117 | A | 118 | A |
| 119 | A | 120 | A |
| 121 | A | 122 | A |
| 123 | A | 124 | A |
| 125 | B | 126 | A |
| 127 | A | 128 | A |
| 129 | B | 130 | A |
| 131 | B | 132 | B |
| 133 | B | 134 | A |
| 135 | B | 136 | C |
| 137 | B | 138 | B |
| 139 | B | 140 | B |
| 141 | B | 142 | B |
| 143 | B | 144 | C |
| 145 | C | 146 | C |
| 147 | C | 148 | B |
| 149 | A | 150 | A |
| 151 | B | 152 | C |
| 153 | A | 154 | B |
| 155 | B | 156 | A |
| 157 | B | 158 | B |
| 159 | B | 160 | A |
| 161 | B | 162 | C |
| 163 | A | 164 | B |
| 165 | B | 166 | C |
| 167 | C | 168 | C |
| 169 | C | 170 | C |
| 171 | C | 172 | C |
| 173 | C | 174 | B |
| 175 | C | 176 | A |
| 177 | A | 178 | C |
| 179 | B | 180 | B |
| 181 | C | 182 | B |
| 183 | A | 184 | C |
| 185 | B | 186 | A |
| 187 | B | 188 | C |
| 189 | A | 190 | B |
| 191 | A | 192 | — |
| 193 | B | 194 | C |
| 195 | B | 196 | B |
| 197 | C | 198 | B |
| 199 | B | 200 | B |
| 201 | — | 202 | B |
| 203 | A | 204 | A |
| 205 | A | 206 | A |
| 207 | A | 208 | C |
| 209 | B | 210 | B |
| 211 | B | 212 | C |
| 213 | B | 214 | B |
| 215 | B | 216 | B |
| 217 | C | 218 | B |
| 219 | C | 220 | B |
| 221 | B | 222 | B |
| 223 | A | 224 | A |
| 225 | B | 226 | — |
| 227 | — | 228 | B |
| 229 | A | 230 | B |
| 231 | B | 232 | B |
| 233 | B | 234 | B |
| 235 | A | 236 | A |
| 237 | A | 238 | B |
| 239 | A | 240 | A |
| 241 | A | 242 | A |
| 243 | A | 244 | A |
| 245 | B | 246 | A |
| 247 | A | 248 | A |
| 249 | A | 250 | A |
| 251 | A | 252 | A |
| 253 | A | 254 | A |
| 255 | A | 256 | A |
| 257 | A | 258 | A |
| 259 | A | 260 | B |
| 261 | B | 262 | — |
| 263 | — | 264 | — |
| 265 | A | 266 | A |
| 267 | A | 268 | A |
| 269 | A | 270 | A |
| 271 | A | 272 | A |
| 273 | A | 274 | A |
| 275 | A | 276 | A |
| 277 | A | 278 | A |
| 279 | A | 280 | B |
| 281 | A | 282 | B |
| 283 | C | 284 | A |
| 285 | C | 286 | B |
| 287 | B | 288 | B |
| 289 | B | 290 | B |
| 291 | A | 292 | A |
| 293 | A | 294 | B |
| 295 | B | 296 | A |
| 297 | A | 298 | B |
| 299 | B | 300 | A |
| 301 | B | 302 | B |
| 303 | B | 304 | C |
| 305 | B | 306 | B |
| 307 | C | 308 | A |
| 309 | B | 310 | A |
| 311 | B | 312 | A |
| 313 | A | 314 | A |
| 315 | — | 316 | A |
| 317 | B | 318 | C |
| 319 | B | 320 | C |
| 321 | B | 322 | B |
| 323 | A | 324 | B |
| 325 | A | 326 | B |
| 327 | B | 328 | B |
| 329 | B | 330 | B |
| 331 | B | 332 | A |
| 333 | A | 334 | A |
| 335 | A | 336 | A |
| 337 | A | 338 | A |
| 339 | A | 340 | A |
| 341 | A | 342 | A |
| 343 | B | 344 | A |
| 345 | A | 346 | B |
| 347 | A | 348 | A |
| 349 | A | 350 | A |
| 351 | A | 352 | A |
| 353 | A | 354 | B |
| 355 | A | 356 | A |
| 357 | A | 358 | A |
| 359 | A | 360 | A |
| 361 | A | 362 | A |
| 363 | A | 364 | A |
| 365 | A | 366 | A |
| 367 | A | 368 | A |
| 369 | B | 370 | — |
| 371 | A | 372 | A |
| 373 | A | 374 | A |
| 375 | A | 376 | A |
| 377 | A | 378 | B |
| 379 | B | 380 | A |
| 381 | A | 382 | A |
| 383 | B | 384 | B |
| 385 | A | 386 | B |
| 387 | B | 388 | A |
| 389 | B | 390 | C |
| 391 | C | 392 | C |
| 393 | A | 394 | C |
| 395 | B | 396 | A |
| 397 | A | 398 | A |
| 399 | C | 400 | B |
| 401 | A | 402 | A |
| 403 | A | 404 | A |
| 405 | A | 406 | A |
| 407 | A | 408 | A |
| 409 | A | 410 | A |
| 411 | A | 412 | A |
| 413 | B | 414 | B |
| 415 | B | 416 | C |
| 417 | A | 418 | A |
| 419 | A | 420 | B |
| 421 | B | 422 | B |
| 423 | A | 424 | A |
| 425 | A | 426 | B |
| 427 | A | 428 | A |
| 429 | A | 430 | A |
| 431 | A | 432 | B |
| 433 | B | 434 | B |
| 435 | A | 436 | A |
| 437 | A | 438 | A |
| 439 | A | 440 | A |
| 441 | A | 442 | A |
| 443 | B | 444 | B |
| 445 | B | 446 | B |
| 447 | C | 448 | C |
| 449 | C | 450 | C |
| 451 | C | 452 | B |
| 453 | A | 454 | A |
| 455 | B | 456 | B |
| 457 | B | 458 | A |
| 459 | A | 460 | A |
| 461 | A | 462 | A |
| 463 | A | 464 | B |
| 465 | B | 466 | B |
| 467 | B | 468 | B |
| 469 | B | 470 | B |
| 471 | B | 472 | B |
| 473 | B | 474 | B |
| 475 | B | 476 | A |
| 477 | B | 478 | A |
| 479 | A | 480 | A |
| 481 | A | 482 | A |
| 483 | A | 484 | B |
| 485 | A | 486 | A |
| 487 | A | 488 | A |
| 489 | A | 490 | A |
| 491 | B | 492 | A |
| 493 | B | 494 | A |
| 495 | A | 496 | A |
| 497 | A | 498 | A |
| 499 | A | 500 | A |
| 501 | A | 502 | B |
| 503 | — | 504 | A |
| 505 | A | 506 | A |
| 507 | A | 508 | A |
| 509 | A | 510 | A |
| 511 | B | 512 | A |
| 513 | A | 514 | A |
| 515 | A | 516 | A |
| 517 | A | 518 | A |
| 519 | A | 520 | B |
| 521 | B | 522 | B |
| 523 | B | 524 | A |
| 525 | C | 526 | A |
| 527 | B | 528 | A |
| 529 | A | 530 | C |
| 531 | A | 532 | A |
| 533 | B | 534 | B |
| 535 | B | 536 | B |
| 537 | B | 538 | C |
| 539 | C | 540 | C |
| 541 | C | 542 | C |
| 543 | C | 544 | B |
| 545 | A | 546 | B |
| 547 | B | 548 | B |
| 549 | B | 550 | C |
| 551 | B | 552 | A |
| 553 | B | 554 | B |
| 555 | B | 556 | B |
| 557 | B | 558 | A |
| 559 | B | 560 | A |
| 561 | B | 562 | A |
| 563 | B | 564 | B |
| 565 | B | 566 | B |
| 567 | B | 568 | B |
| 569 | A | 570 | C |
| 571 | B | 572 | B |
| 573 | A | 574 | A |
| 575 | A | 576 | A |
| 577 | C | 578 | B |
| 579 | B | 580 | A |
| 581 | B | 582 | B |
| 583 | B | 584 | B |
| 585 | B | 586 | B |
| 587 | B | 588 | B |
| 589 | A | 590 | B |
| 591 | B | 592 | B |
| 593 | B | 594 | B |
| 595 | A | 596 | C |
| 597 | C | 598 | C |
| 599 | C | 600 | C |
| 601 | B | 602 | A |
| 603 | B | 604 | A |
| 605 | — | 606 | A |
| 607 | C | 608 | B |

HDV Infection in HepG2-NTCP Cells and RNA Quantification

HDV virus collected from the supernatants of HuH7-END cells was purified in the presence of 6% polyethylene glycol (PEG). Viral titer was then quantified by RT-qPCR using HDV specific primers against a reference standard. For HDV infection, HepG2-NTCP cells were seeded in 96-well plates at 60,000 cells/well. On the day of infection, compounds were 4-fold serially diluted in DMSO and pre-incubated with HepG2-NTCP cells for two hours before infecting with 800 GE/cell of HDV in 4% PEG. On day one post infection, HDV-containing media was aspirated, cells were washed once with PBS and then maintained in 2.5% DMSO containing media for the remainder of the assay. Viral RNA was isolated using RNeasy kits (Qiagen) at day 12 post infection and reverse transcribed into cDNA using High-Capacity RNA-to-cDNA Kit (Thermo Fisher). Relative HDV gene expression was quantified by RT-qPCR after normalization to expression of a housekeeping gene. Irrespective of readout, compound concentrations that reduce viral RNA by 50% relative to DMSO controls (EC₅₀) are reported. EC₅₀ ranges are as follows: A<0.1 μM; B 0.1-1 μM; C>1 μM.

TABLE 2
Summary of HepG2-NTCP HDV Activities

| Example Number | Cellular EC₅₀ (μM) |
|---|---|
| 25 | A |
| 116 | A |
| 126 | A |

HDV Infection in Primary Human Hepatocytes (PHHs) and RNA Quantification

Primary human hepatocytes (Thermo Fisher Scientific) were thawed in Williams E Medium with CM3000 supplement pack (Thermo Fisher Scientific) and seeded at 70,000 cells/well on collagen-coated 96-well plate. Six hours post-seeding, cell medium was changed to Williams E Medium with CM4000 cell maintenance supplement pack for further culture (Thermo Fisher Scientific). HDV virus collected from the supernatants of HuH7-END cells was purified in the presence of 6% polyethylene glycol (PEG). Viral titer was then quantified by RT-qPCR using HDV specific primers against a reference standard. On the day of infection, compounds were 4-fold serially diluted in DMSO and pre-incubated with PHHs for two hours before infecting with 100 GE/cell of HDV in 4% PEG. On day one post infection, HDV-containing media was aspirated, cells were washed once with PBS and then maintained in 2.5% DMSO containing media for the remainder of the assay. Viral RNA was isolated using RNeasy kits at day 8 post infection and reverse transcribed into cDNA using High-Capacity RNA-to-cDNA Kit. Relative HDV gene expression was quantified by RT-qPCR after normalization to expression of a housekeeping gene. Irrespective of readout, compound concentrations that reduce viral RNA by 50% relative to DMSO controls (EC50) are reported. EC₅₀ ranges are as follows: A<0.1 μM; B 0.1-1 μM; C>1 μM.

TABLE 3
Summary of Primary Human Hepatocyte HDV Activities

| | Cellular EC₅₀ (μM) | Cellular EC₅₀ (μM) |
|---|---|---|
| Example Number | Donor 1 | Donor 2 |
| 25 | A | B |
| 116 | A | — |
| 127 | — | A |
| 128 | — | A |
| 336 | A | — |
| 353 | A | — |
| 372 | A | — |
| 376 | A | — |
| 514 | A | A |

HepG2-NTCP preS1 Binding Competition Assay

Myristoylated preS1 peptide (2-48 aa) conjugated to a C-terminal FITC tag was synthesized to evaluate preS1 binding to NTCP-expressing cells. HepG2-NTCP cells seeded in 384-wells were pre-treated with compounds for 2 hours prior to the addition of FITC-labeled preS1 peptide. After co-incubation for 30 minutes, unbound FITC-preS1 peptide was washed twice with PBS, and the fluorescence of preS1-FITC bound to cell surface was detected by Envision plate reader. EC₅₀ ranges are as follows: A<0.1 μM; B 0.1-1 μM; C>1 μM.

TABLE 4
Summary of HepG2-NTPC preS1 binding competition activities

| Example Number | EC₅₀ (μM) |
|---|---|
| 116 | A |
| 127 | A |
| 157 | B |
| 192 | B |
| 201 | C |
| 225 | B |
| 226 | B |
| 227 | B |
| 235 | A |
| 239 | A |
| 240 | A |
| 241 | A |
| 242 | A |
| 246 | A |
| 262 | A |
| 263 | A |
| 264 | A |
| 273 | A |
| 274 | A |
| 275 | A |
| 304 | C |
| 307 | C |
| 315 | C |
| 316 | A |
| 317 | B |
| 318 | C |
| 320 | C |
| 321 | B |
| 323 | A |
| 325 | A |
| 348 | A |
| 370 | B |
| 388 | A |
| 391 | C |
| 486 | A |
| 503 | B |
| 512 | A |
| 563 | A |
| 595 | A |
| 605 | B |
| 608 | B |

While this invention has been particularly shown and described with references to preferred embodiments thereof, it will be understood by those skilled in the art that various changes in form and details may be made therein without departing from the scope of the invention encompassed by the appended claims.

1. A compound represented by Formula (I): 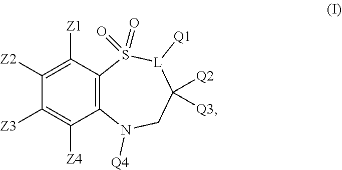
or a pharmaceutically acceptable salt thereof, wherein:
Q1, Q2, Q3, an Q4 are each independently selected from hydrogen, optionally substituted —C₁-C₆ alkyl, optionally substituted —C₂-C₆ alkenyl, optionally substituted —C₁-C₆ alkoxy, optionally substituted —C₃-C₈ cycloalkyl, optionally substituted —C₃-C₈ cycloalkenyl, optionally substituted 3- to 8-membered heterocycloalkyl, optionally substituted aryl, and optionally substituted heteroaryl;
alternatively, Q1 and Q2 are taken together with the atoms to which they are attached to form an optionally substituted 3-8 membered heterocyclic or carbocyclic ring containing 0, 1, 2, or 3 double bonds;
alternatively, Q2 and Q3 are taken together with the carbon atom to which they are attached to form an optionally substituted 3-8 membered heterocyclic or carbocyclic ring containing 0, 1, 2, or 3 double bonds;
L is CR₁₄ or N;
Z1, Z3, and Z4 are each independently selected from:
1) hydrogen;
2) halogen;
3) —NO₂;
4) Cyano;
5) Optionally substituted —C₁-C₈ alkyl;
6) Optionally substituted —C₂-C₈ alkenyl;
7) Optionally substituted —C₂-C₈ alkynyl;
8) Optionally substituted —C₃-C₅ cycloalkyl;
9) Optionally substituted 3- to 12-membered heterocycloalkyl;
10) Optionally substituted aryl;
11) Optionally substituted arylalkyl;
12) Optionally substituted heteroaryl;
13) Optionally substituted heteroarylalkyl;
14) —SR₁₁;
15) —S(O)₂R₁₁;
16) —S(O)₂N(R₁₁)(R₁₂);
17) —C(O)R₁₁;
18) —C(O)OR₁₁;
19) —C(O)N(R₁₁)(R₁₂);
20) —C(O)N(R₁₁)S(O)₂(R₁₂);
21) —N(R₁₁)(R₁₂);
22) —N(R₁₃)C(O)N(R₁₁)(R₁₂);
23) —N(R₁₁)C(O)(R₁₂);
24) —N(R₁₁)C(O)₂(R₁₂);
25) —N(R₁₃)S(O)₂N(R₁₁)(R₁₂);
26) —N(R₁₁)S(O)₂(R₁₂);
27) —OR₁₁;
28) —OC(O)R₁₁;
29) —OC(O)OR₁₁; and
30) —OC(O)N(R₁₁)(R₁₂);
Z2 is selected from:
1) Optionally substituted —C₃-C₈ cycloalkyl;
2) Optionally substituted 3- to 12-membered heterocycloalkyl;
3) Optionally substituted aryl;
4) Optionally substituted arylalkyl;
5) Optionally substituted heteroaryl; and
6) Optionally substituted heteroarylalkyl;
R₁₁, R₁₂, and R₁₃, are each independently selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl; alternatively, R₁₁ and R₁₂ are taken together with the nitrogen atom to which they attached to form an optionally substituted 3-8 membered heterocyclic containing 0, 1, 2, or 3 double bonds; and R₁₄ is hydrogen, optionally substituted —C₁-C₆ alkyl, optionally substituted —C₂-C₆ alkenyl, optionally substituted —C₂-C₆ alkynyl, or optionally substituted —C₁-C₆ alkoxy.

2. The compound of claim 1, wherein Z2 is optionally substituted aryl or optionally substituted heteroaryl.

3. The compound of claim 1, represented by one of Formula (V-1) or Formula (V-2), or a pharmaceutically acceptable salt thereof: 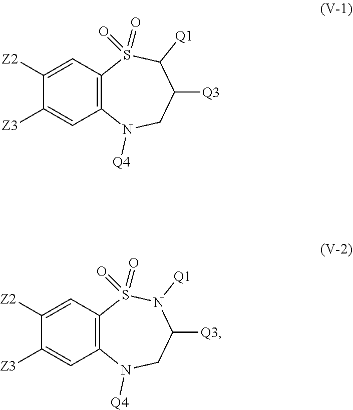
wherein Z2, Z3, Q1, Q3, and Q4 are as defined in claim 1.

4. The compound of claim 1, represented by one of Formulae (IX-1) to (IX-6), or a pharmaceutically acceptable salt thereof: 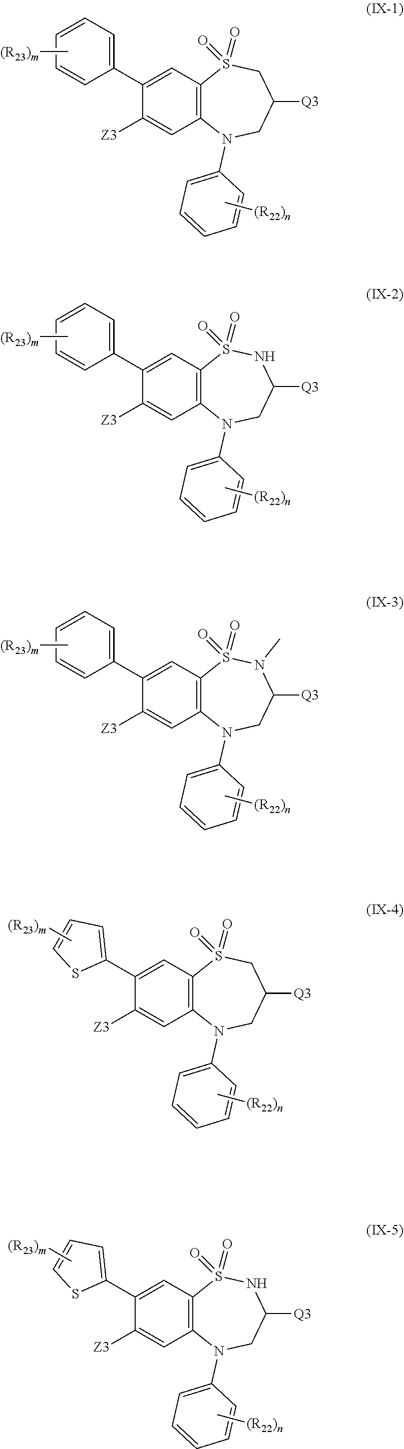 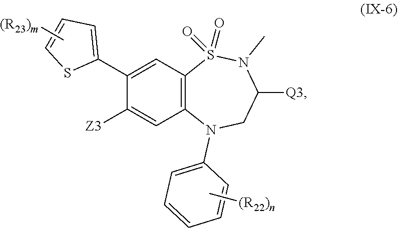
wherein n is 0, 1, 2, or 3; m is 0, 1, 2, or 3;
each R₂₂ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
each R₂₃ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) —CO₂H;
9) —SO₃H
10) —PO₃H₂
11) —NHC(O)OR₁₁;
12) —NHS(O)₂R₁₁;
13) —NHC(O)R₁₁;
14) —SO₂NHC(O)R₁₁;
15) optionally substituted —C₁-C₆ alkyl;
16) optionally substituted —C₃-C₈ cycloalkyl;
17) optionally substituted 3- to 8-membered heterocycloalkyl;
18) optionally substituted aryl; and
19) optionally substituted heteroaryl;
Z3, Q3, R₁₁, and R₁₂ are as defined in claim 1.

5. The compound of claim 1, represented by Formula (XVII), 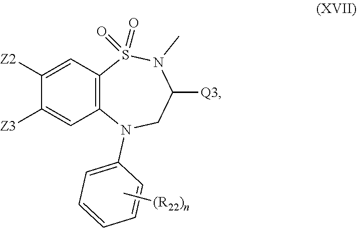
wherein n is 0, 1, 2, or 3;
each R₂₂ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) optionally substituted —C₁-C₆ alkyl;
9) optionally substituted —C₃-C₈ cycloalkyl;
10) optionally substituted 3- to 8-membered heterocycloalkyl;
11) optionally substituted aryl; and
12) optionally substituted heteroaryl;
and Z2, Z3, Q3, R₁₁, and R₁₂ are as defined in claim 1.

6. The compound of claim 1, represented by one of Formulae (XX-1) to (XX-4), 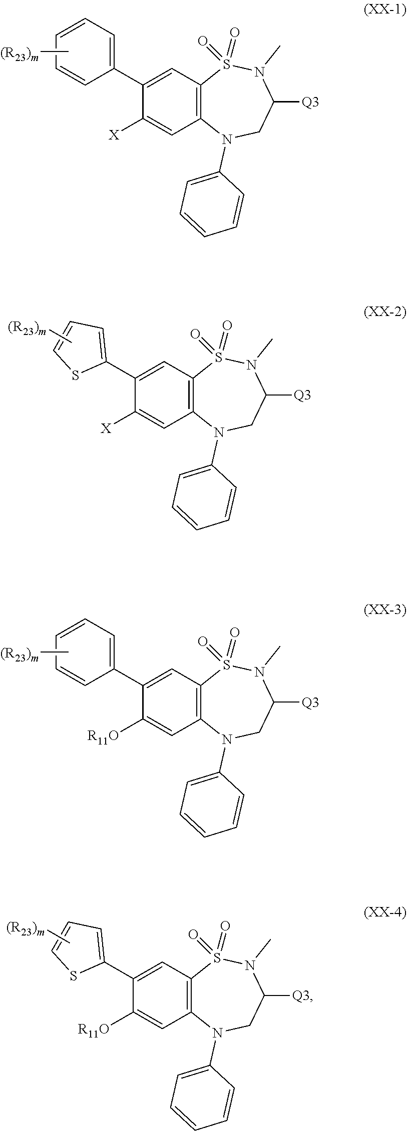
wherein X is halogen; m is 0, 1, 2, or 3;
each R₂₃ is independently selected from:
1) halogen;
2) —CN;
3) —NO₂;
4) —OR₁₁;
5) —SR₁₁;
6) —NR₁₁R₁₂;
7) —OC(O)NR₁₁R₁₂;
8) —CO₂H;
9) SO₃H
10) —PO₃H₂
11) —NHC(O)OR₁₁;
12) —NHS(O)₂R₁₁;
13) N₂C(O)R₁₁
14) optionally substituted —C₁-C₆ alkyl;
15) optionally substituted —C₃-C₈ cycloalkyl;
16) optionally substituted 3- to 8-membered heterocycloalkyl;
17) optionally substituted aryl; and
18) optionally substituted heteroaryl;
Q3, R₁₁, and R₁₂ are as defined in claim 1.

7. The compound of claim 1, selected from the compounds set forth below or a pharmaceutically acceptable salt thereof:

| Compound | Structure |
|---|---|
| 1 | 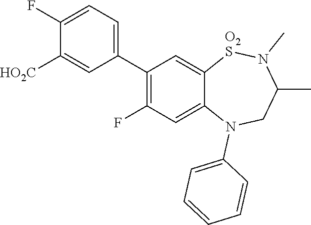 [SMILES: CC1CN(c2ccccc2)c2cc(F)c(-c3ccc(F)c(C(=O)O)c3)cc2S(=O)(=O)N1C] |
| 2 | 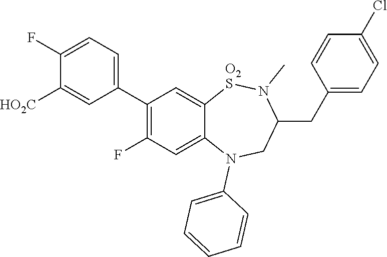 [SMILES: CN1C(Cc2ccc(Cl)cc2)CN(c2ccccc2)c2cc(F)c(-c3ccc(F)c(C(=O)O)c3)cc2S1(=O)=O] |
| 3 | 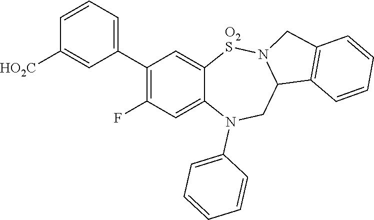 [SMILES: O=C(O)c1cccc(-c2cc3c(cc2F)N(c2ccccc2)CC2c4ccccc4CN2S3(=O)=O)c1] |
| 4 | 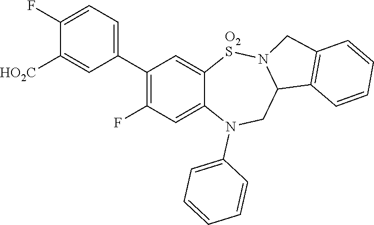 [SMILES: O=C(O)c1cc(-c2cc3c(cc2F)N(c2ccccc2)CC2c4ccccc4CN2S3(=O)=O)ccc1F] |
| 5 | 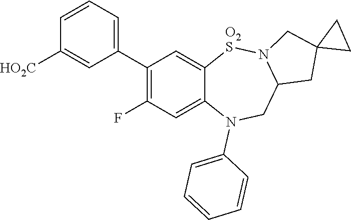 [SMILES: O=C(O)c1cccc(-c2cc3c(cc2F)N(c2ccccc2)CC2CC4(CC4)CN2S3(=O)=O)c1] |
| 6 | 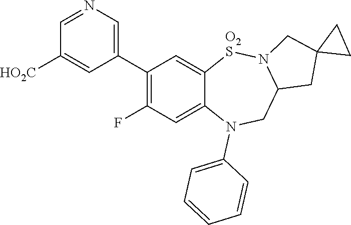 [SMILES: O=C(O)c1cncc(-c2cc3c(cc2F)N(c2ccccc2)CC2CC4(CC4)CN2S3(=O)=O)c1] |
| 7 | 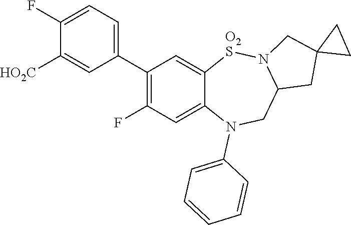 [SMILES: O=C(O)c1cc(-c2cc3c(cc2F)N(c2ccccc2)CC2CC4(CC4)CN2S3(=O)=O)ccc1F] |
| 8 | 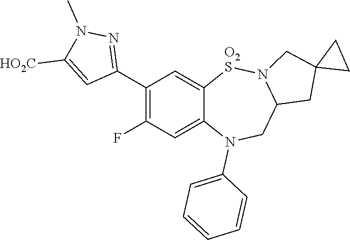 [SMILES: Cn1nc(-c2cc3c(cc2F)N(c2ccccc2)CC2CC4(CC4)CN2S3(=O)=O)cc1C(=O)O] |
| 9 | 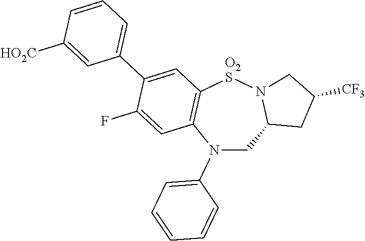 [SMILES: O=C(O)c1cccc(-c2cc3c(cc2F)N(c2ccccc2)C[C@H]2C[C@@H](C(F)(F)F)CN2S3(=O)=O)c1] |
| 10 | 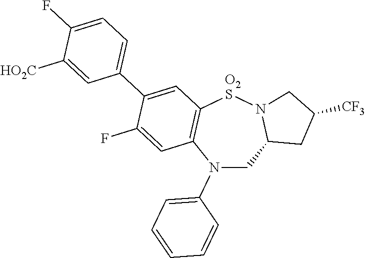 [SMILES: O=C(O)c1cc(-c2cc3c(cc2F)N(c2ccccc2)C[C@H]2C[C@@H](C(F)(F)F)CN2S3(=O)=O)ccc1F] |
| 11 | 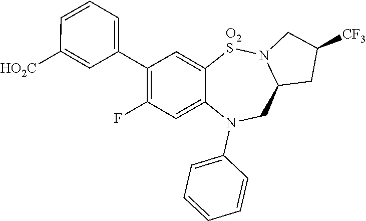 [SMILES: O=C(O)c1cccc(-c2cc3c(cc2F)N(c2ccccc2)C[C@@H]2C[C@H](C(F)(F)F)CN2S3(=O)=O)c1] |
| 12 | 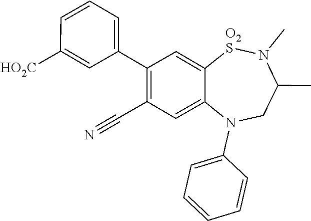 [SMILES: CC1CN(c2ccccc2)c2cc(C#N)c(-c3cccc(C(=O)O)c3)cc2S(=O)(=O)N1C] |
| 13 | 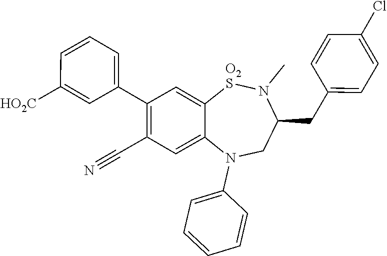 [SMILES: CN1[C@@H](Cc2ccc(Cl)cc2)CN(c2ccccc2)c2cc(C#N)c(-c3cccc(C(=O)O)c3)cc2S1(=O)=O] |
| 14 | 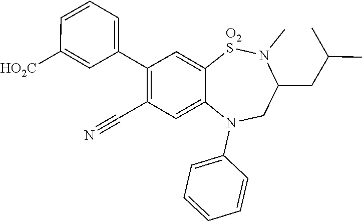 [SMILES: CC(C)CC1CN(c2ccccc2)c2cc(C#N)c(-c3cccc(C(=O)O)c3)cc2S(=O)(=O)N1C] |
| 15 | 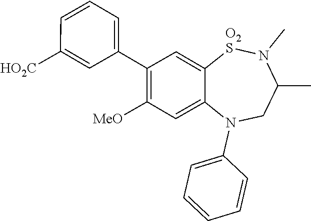 [SMILES: COc1cc2c(cc1-c1cccc(C(=O)O)c1)S(=O)(=O)N(C)C(C)CN2c1ccccc1] |
| 16 | 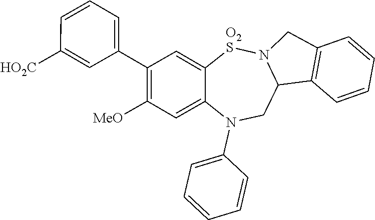 [SMILES: COc1cc2c(cc1-c1cccc(C(=O)O)c1)S(=O)(=O)N1Cc3ccccc3C1CN2c1ccccc1] |
| 17 | 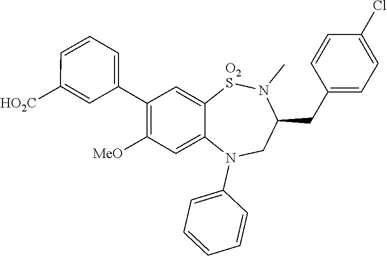 [SMILES: COc1cc2c(cc1-c1cccc(C(=O)O)c1)S(=O)(=O)N(C)[C@@H](Cc1ccc(Cl)cc1)CN2c1ccccc1] |
| 18 | 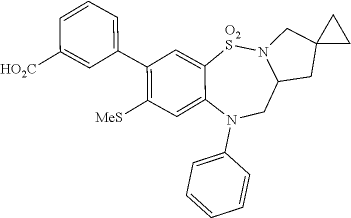 [SMILES: CSc1cc2c(cc1-c1cccc(C(=O)O)c1)S(=O)(=O)N1CC3(CC3)CC1CN2c1ccccc1] |
| 19 | 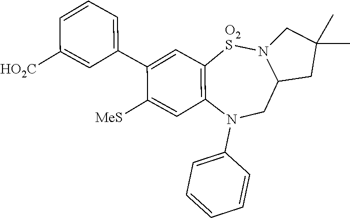 [SMILES: CSc1cc2c(cc1-c1cccc(C(=O)O)c1)S(=O)(=O)N1CC(C)(C)CC1CN2c1ccccc1] |
| 20 | 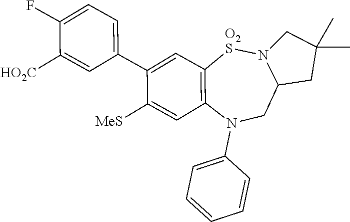 [SMILES: CSc1cc2c(cc1-c1ccc(F)c(C(=O)O)c1)S(=O)(=O)N1CC(C)(C)CC1CN2c1ccccc1] |
| 21 | 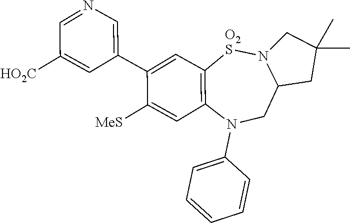 [SMILES: CSc1cc2c(cc1-c1cncc(C(=O)O)c1)S(=O)(=O)N1CC(C)(C)CC1CN2c1ccccc1] |
| 22 | 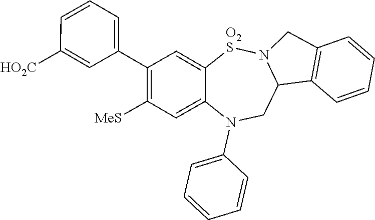 [SMILES: CSc1cc2c(cc1-c1cccc(C(=O)O)c1)S(=O)(=O)N1Cc3ccccc3C1CN2c1ccccc1] |
| 23 | 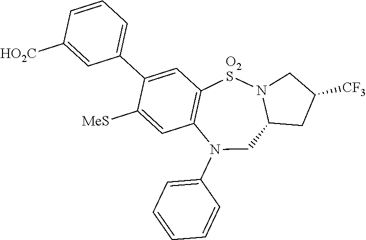 [SMILES: CSc1cc2c(cc1-c1cccc(C(=O)O)c1)S(=O)(=O)N1C[C@H](C(F)(F)F)C[C@@H]1CN2c1ccccc1] |
| 24 | 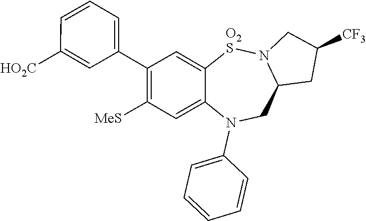 [SMILES: CSc1cc2c(cc1-c1cccc(C(=O)O)c1)S(=O)(=O)N1C[C@@H](C(F)(F)F)C[C@H]1CN2c1ccccc1] |
| 25 | 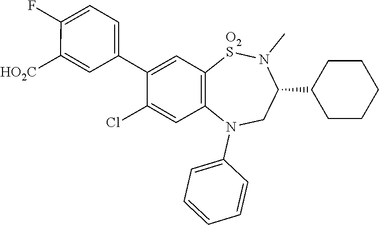 [SMILES: CN1[C@H](C2CCCCC2)CN(c2ccccc2)c2cc(Cl)c(-c3ccc(F)c(C(=O)O)c3)cc2S1(=O)=O] |
| 26 | 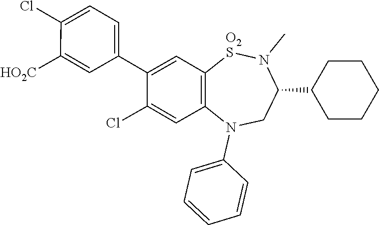 [SMILES: CN1[C@H](C2CCCCC2)CN(c2ccccc2)c2cc(Cl)c(-c3ccc(Cl)c(C(=O)O)c3)cc2S1(=O)=O] |
| 27 | 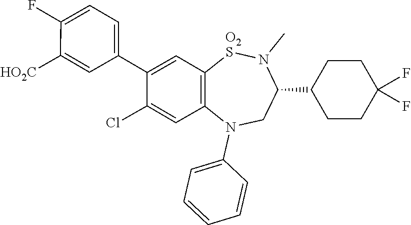 [SMILES: CN1[C@H](C2CCC(F)(F)CC2)CN(c2ccccc2)c2cc(Cl)c(-c3ccc(F)c(C(=O)O)c3)cc2S1(=O)=O] |
| 28 | 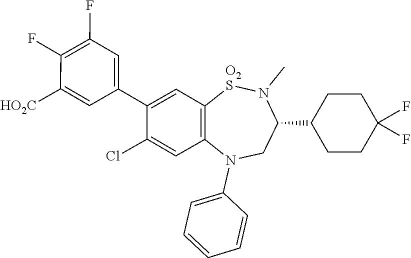 [SMILES: CN1[C@H](C2CCC(F)(F)CC2)CN(c2ccccc2)c2cc(Cl)c(-c3cc(F)c(F)c(C(=O)O)c3)cc2S1(=O)=O] |
| 29 | 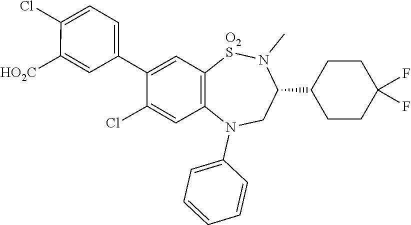 [SMILES: CN1[C@H](C2CCC(F)(F)CC2)CN(c2ccccc2)c2cc(Cl)c(-c3ccc(Cl)c(C(=O)O)c3)cc2S1(=O)=O] |
| 30 | 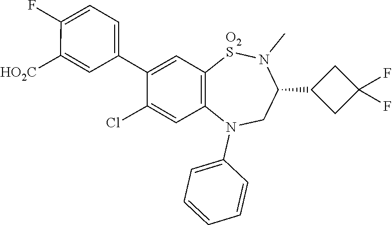 [SMILES: CN1[C@H](C2CC(F)(F)C2)CN(c2ccccc2)c2cc(Cl)c(-c3ccc(F)c(C(=O)O)c3)cc2S1(=O)=O] |
| 31 | 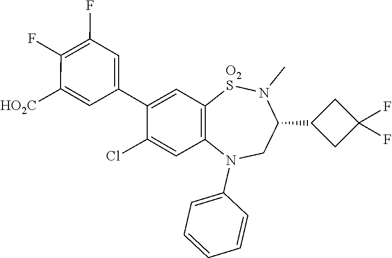 [SMILES: CN1[C@H](C2CC(F)(F)C2)CN(c2ccccc2)c2cc(Cl)c(-c3cc(F)c(F)c(C(=O)O)c3)cc2S1(=O)=O] |
| 32 | 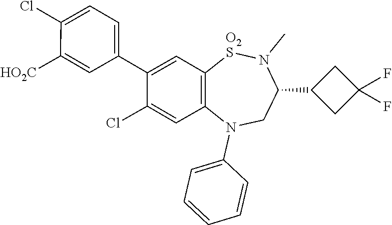 [SMILES: CN1[C@H](C2CC(F)(F)C2)CN(c2ccccc2)c2cc(Cl)c(-c3ccc(Cl)c(C(=O)O)c3)cc2S1(=O)=O] |
| 33 | 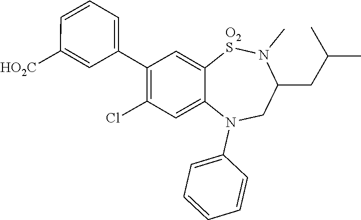 [SMILES: CC(C)CC1CN(c2ccccc2)c2cc(Cl)c(-c3cccc(C(=O)O)c3)cc2S(=O)(=O)N1C] |
| 34 | 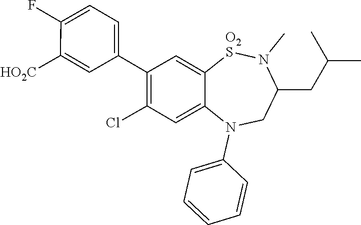 [SMILES: CC(C)CC1CN(c2ccccc2)c2cc(Cl)c(-c3ccc(F)c(C(=O)O)c3)cc2S(=O)(=O)N1C] |
| 35 | 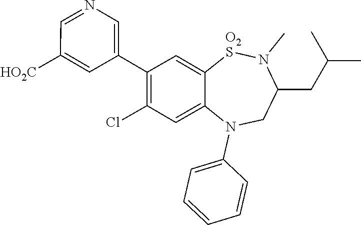 [SMILES: CC(C)CC1CN(c2ccccc2)c2cc(Cl)c(-c3cncc(C(=O)O)c3)cc2S(=O)(=O)N1C] |
| 36 | 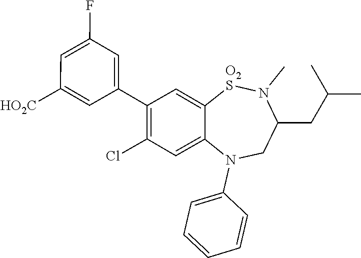 [SMILES: CC(C)CC1CN(c2ccccc2)c2cc(Cl)c(-c3cc(F)cc(C(=O)O)c3)cc2S(=O)(=O)N1C] |
| 37 | 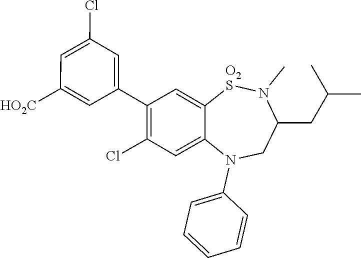 [SMILES: CC(C)CC1CN(c2ccccc2)c2cc(Cl)c(-c3cc(Cl)cc(C(=O)O)c3)cc2S(=O)(=O)N1C] |
| 38 | 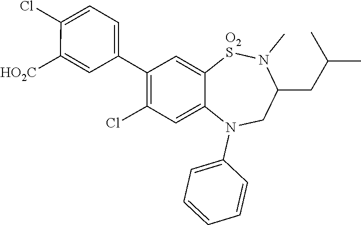 [SMILES: CC(C)CC1CN(c2ccccc2)c2cc(Cl)c(-c3ccc(Cl)c(C(=O)O)c3)cc2S(=O)(=O)N1C] |
| 39 | 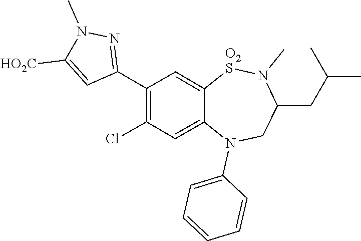 [SMILES: CC(C)CC1CN(c2ccccc2)c2cc(Cl)c(-c3cc(C(=O)O)n(C)n3)cc2S(=O)(=O)N1C] |
| 40 | 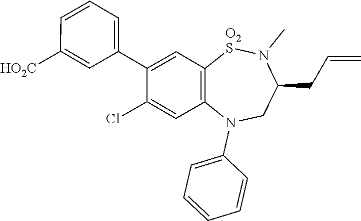 [SMILES: C=CC[C@H]1CN(c2ccccc2)c2cc(Cl)c(-c3cccc(C(=O)O)c3)cc2S(=O)(=O)N1C] |
| 41 | 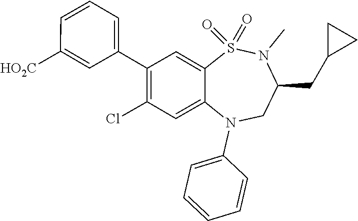 [SMILES: CN1[C@@H](CC2CC2)CN(c2ccccc2)c2cc(Cl)c(-c3cccc(C(=O)O)c3)cc2S1(=O)=O] |
| 42 | 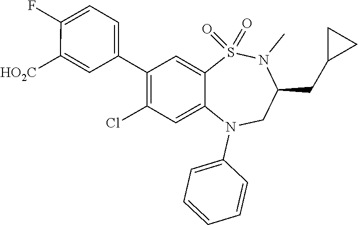 [SMILES: CN1[C@@H](CC2CC2)CN(c2ccccc2)c2cc(Cl)c(-c3ccc(F)c(C(=O)O)c3)cc2S1(=O)=O] |
| 43 | 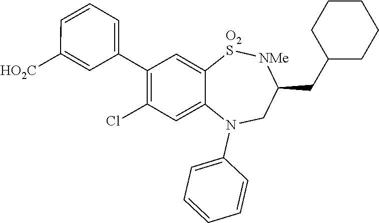 [SMILES: CN1[C@@H](CC2CCCCC2)CN(c2ccccc2)c2cc(Cl)c(-c3cccc(C(=O)O)c3)cc2S1(=O)=O] |
| 44 | 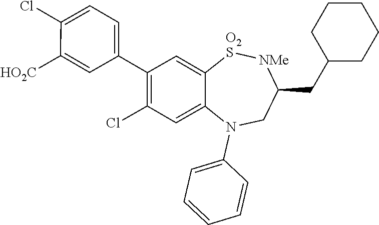 [SMILES: CN1[C@@H](CC2CCCCC2)CN(c2ccccc2)c2cc(Cl)c(-c3ccc(Cl)c(C(=O)O)c3)cc2S1(=O)=O] |
| 45 | 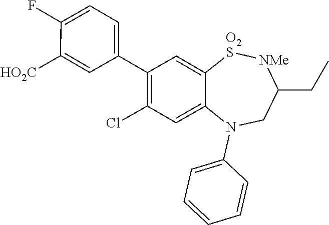 [SMILES: CCC1CN(c2ccccc2)c2cc(Cl)c(-c3ccc(F)c(C(=O)O)c3)cc2S(=O)(=O)N1C] |
| 46 | 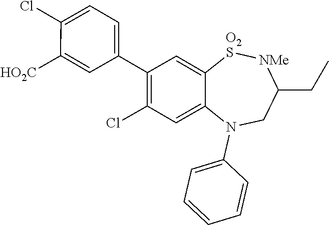 [SMILES: CCC1CN(c2ccccc2)c2cc(Cl)c(-c3ccc(Cl)c(C(=O)O)c3)cc2S(=O)(=O)N1C] |
| 47 | 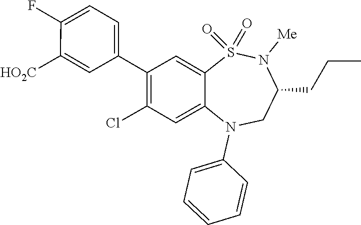 [SMILES: CCC[C@@H]1CN(c2ccccc2)c2cc(Cl)c(-c3ccc(F)c(C(=O)O)c3)cc2S(=O)(=O)N1C] |
| 48 | 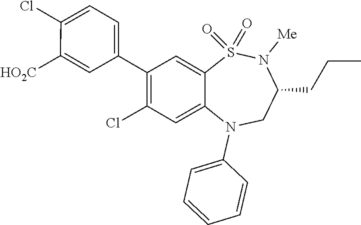 [SMILES: CCC[C@@H]1CN(c2ccccc2)c2cc(Cl)c(-c3ccc(Cl)c(C(=O)O)c3)cc2S(=O)(=O)N1C] |
| 49 | 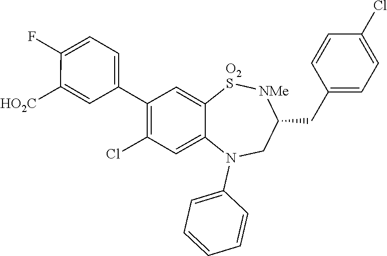 [SMILES: CN1[C@H](Cc2ccc(Cl)cc2)CN(c2ccccc2)c2cc(Cl)c(-c3ccc(F)c(C(=O)O)c3)cc2S1(=O)=O] |
| 50 | 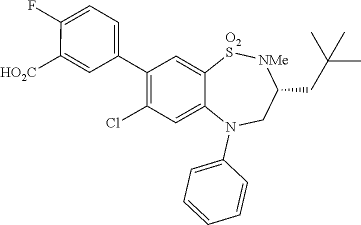 [SMILES: CN1[C@H](CC(C)(C)C)CN(c2ccccc2)c2cc(Cl)c(-c3ccc(F)c(C(=O)O)c3)cc2S1(=O)=O] |
| 51 | 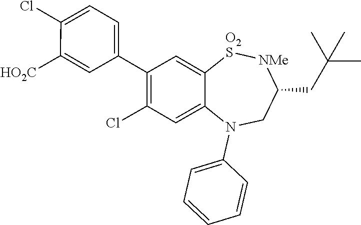 [SMILES: CN1[C@H](CC(C)(C)C)CN(c2ccccc2)c2cc(Cl)c(-c3ccc(Cl)c(C(=O)O)c3)cc2S1(=O)=O] |
| 52 | 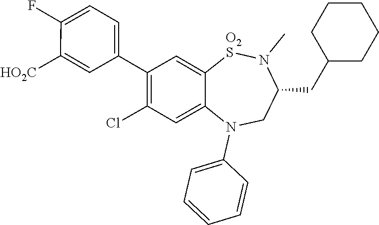 [SMILES: CN1[C@H](CC2CCCCC2)CN(c2ccccc2)c2cc(Cl)c(-c3ccc(F)c(C(=O)O)c3)cc2S1(=O)=O] |
| 53 | 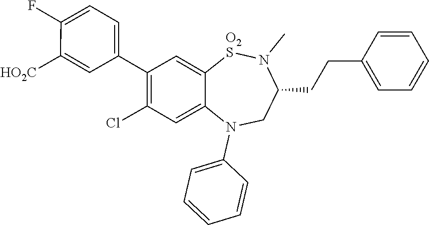 [SMILES: CN1[C@H](CCc2ccccc2)CN(c2ccccc2)c2cc(Cl)c(-c3ccc(F)c(C(=O)O)c3)cc2S1(=O)=O] |
| 54 | 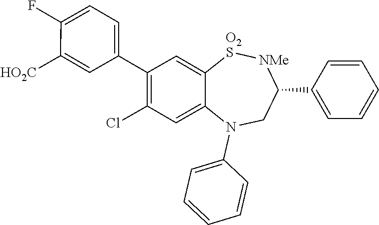 [SMILES: CN1[C@H](c2ccccc2)CN(c2ccccc2)c2cc(Cl)c(-c3ccc(F)c(C(=O)O)c3)cc2S1(=O)=O] |
| 55 | 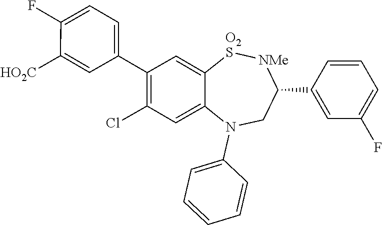 [SMILES: CN1[C@H](c2cccc(F)c2)CN(c2ccccc2)c2cc(Cl)c(-c3ccc(F)c(C(=O)O)c3)cc2S1(=O)=O] |
| 56 | 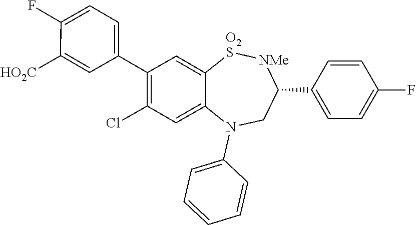 [SMILES: CN1[C@H](c2ccc(F)cc2)CN(c2ccccc2)c2cc(Cl)c(-c3ccc(F)c(C(=O)O)c3)cc2S1(=O)=O] |
| 57 | 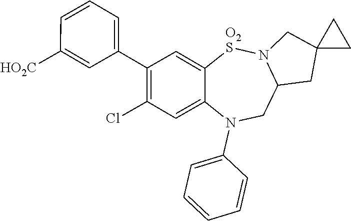 [SMILES: O=C(O)c1cccc(-c2cc3c(cc2Cl)N(c2ccccc2)CC2CC4(CC4)CN2S3(=O)=O)c1] |
| 58 | 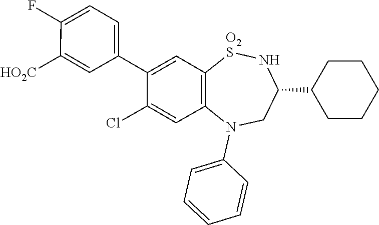 [SMILES: O=C(O)c1cc(-c2cc3c(cc2Cl)N(c2ccccc2)C[C@@H](C2CCCCC2)NS3(=O)=O)ccc1F] |
| 59 | 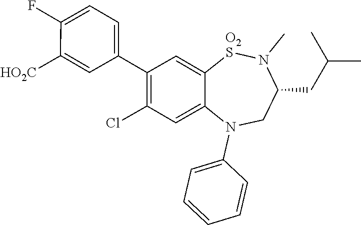 [SMILES: CC(C)C[C@@H]1CN(c2ccccc2)c2cc(Cl)c(-c3ccc(F)c(C(=O)O)c3)cc2S(=O)(=O)N1C] |
| 60 | 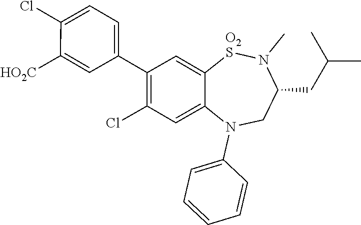 [SMILES: CC(C)C[C@@H]1CN(c2ccccc2)c2cc(Cl)c(-c3ccc(Cl)c(C(=O)O)c3)cc2S(=O)(=O)N1C] |
| 61 | 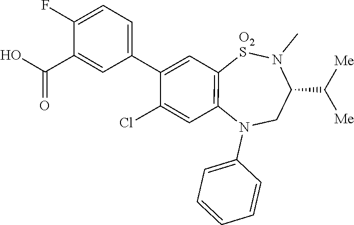 [SMILES: CC(C)[C@@H]1CN(c2ccccc2)c2cc(Cl)c(-c3ccc(F)c(C(=O)O)c3)cc2S(=O)(=O)N1C] |
| 62 | 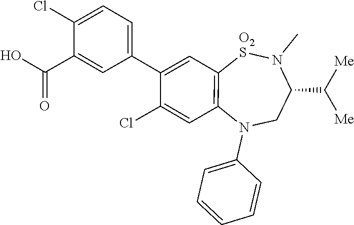 [SMILES: CC(C)[C@@H]1CN(c2ccccc2)c2cc(Cl)c(-c3ccc(Cl)c(C(=O)O)c3)cc2S(=O)(=O)N1C] |
| 63 | 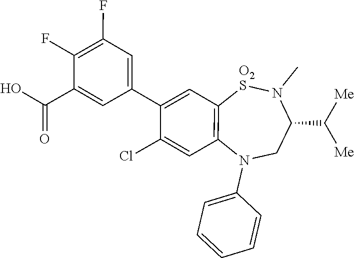 [SMILES: CC(C)[C@@H]1CN(c2ccccc2)c2cc(Cl)c(-c3cc(F)c(F)c(C(=O)O)c3)cc2S(=O)(=O)N1C] |
| 64 | 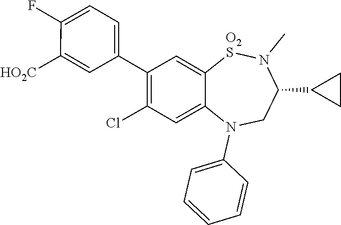 [SMILES: CN1[C@H](C2CC2)CN(c2ccccc2)c2cc(Cl)c(-c3ccc(F)c(C(=O)O)c3)cc2S1(=O)=O] |
| 65 | 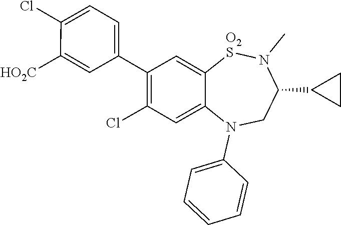 [SMILES: CN1[C@H](C2CC2)CN(c2ccccc2)c2cc(Cl)c(-c3ccc(Cl)c(C(=O)O)c3)cc2S1(=O)=O] |
| 66 | 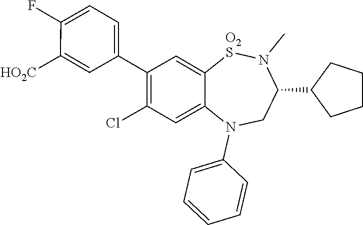 [SMILES: CN1[C@H](C2CCCC2)CN(c2ccccc2)c2cc(Cl)c(-c3ccc(F)c(C(=O)O)c3)cc2S1(=O)=O] |
| 67 | 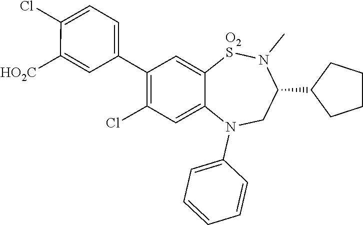 [SMILES: CN1[C@H](C2CCCC2)CN(c2ccccc2)c2cc(Cl)c(-c3ccc(Cl)c(C(=O)O)c3)cc2S1(=O)=O] |
| 68 | 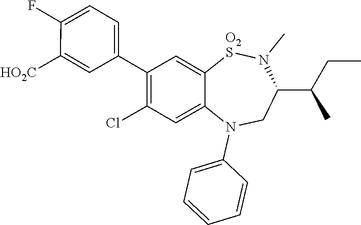 [SMILES: CC[C@@H](C)[C@@H]1CN(c2ccccc2)c2cc(Cl)c(-c3ccc(F)c(C(=O)O)c3)cc2S(=O)(=O)N1C] |
| 69 | 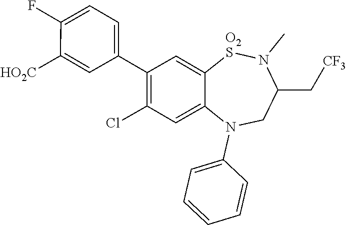 [SMILES: CN1C(CC(F)(F)F)CN(c2ccccc2)c2cc(Cl)c(-c3ccc(F)c(C(=O)O)c3)cc2S1(=O)=O] |
| 70 | 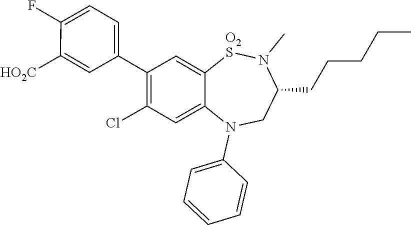 [SMILES: CCCCC[C@@H]1CN(c2ccccc2)c2cc(Cl)c(-c3ccc(F)c(C(=O)O)c3)cc2S(=O)(=O)N1C] |
| 71 | 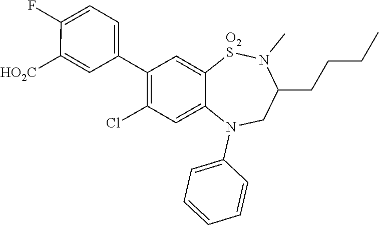 [SMILES: CCCCC1CN(c2ccccc2)c2cc(Cl)c(-c3ccc(F)c(C(=O)O)c3)cc2S(=O)(=O)N1C] |
| 72 | 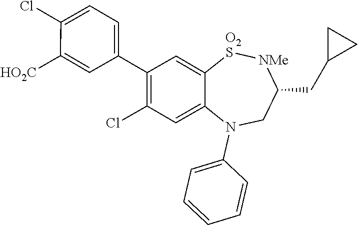 [SMILES: CN1[C@H](CC2CC2)CN(c2ccccc2)c2cc(Cl)c(-c3ccc(Cl)c(C(=O)O)c3)cc2S1(=O)=O] |
| 73 | 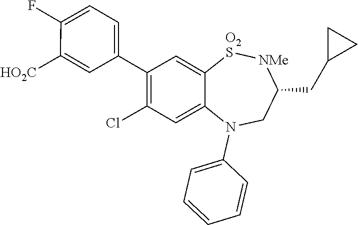 [SMILES: CN1[C@H](CC2CC2)CN(c2ccccc2)c2cc(Cl)c(-c3ccc(F)c(C(=O)O)c3)cc2S1(=O)=O] |
| 74 | 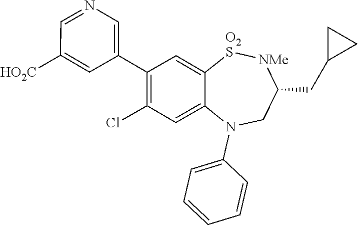 [SMILES: CN1[C@H](CC2CC2)CN(c2ccccc2)c2cc(Cl)c(-c3cncc(C(=O)O)c3)cc2S1(=O)=O] |
| 75 | 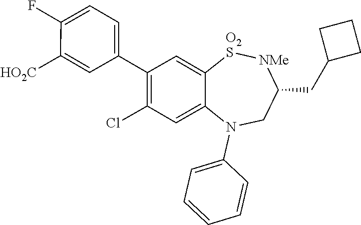 [SMILES: CN1[C@H](CC2CCC2)CN(c2ccccc2)c2cc(Cl)c(-c3ccc(F)c(C(=O)O)c3)cc2S1(=O)=O] |
| 76 | 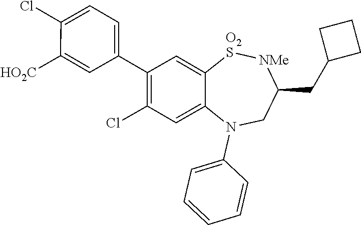 [SMILES: CN1[C@@H](CC2CCC2)CN(c2ccccc2)c2cc(Cl)c(-c3ccc(Cl)c(C(=O)O)c3)cc2S1(=O)=O] |
| 77 | 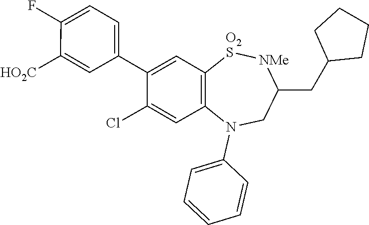 [SMILES: CN1C(CC2CCCC2)CN(c2ccccc2)c2cc(Cl)c(-c3ccc(F)c(C(=O)O)c3)cc2S1(=O)=O] |
| 78 | 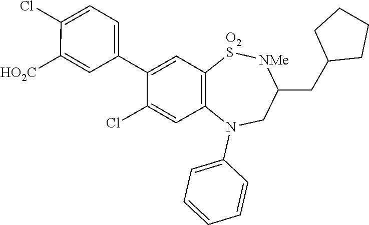 [SMILES: CN1C(CC2CCCC2)CN(c2ccccc2)c2cc(Cl)c(-c3ccc(Cl)c(C(=O)O)c3)cc2S1(=O)=O] |
| 79 | 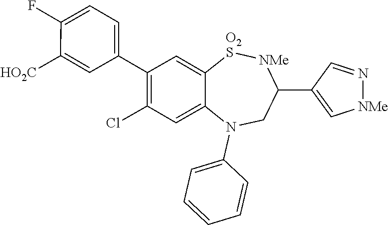 [SMILES: CN1C(c2cnn(C)c2)CN(c2ccccc2)c2cc(Cl)c(-c3ccc(F)c(C(=O)O)c3)cc2S1(=O)=O] |
| 80 | 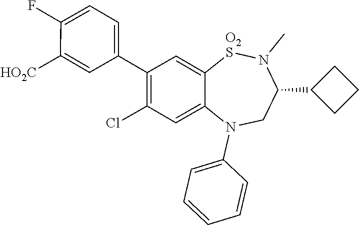 [SMILES: CN1[C@H](C2CCC2)CN(c2ccccc2)c2cc(Cl)c(-c3ccc(F)c(C(=O)O)c3)cc2S1(=O)=O] |
| 81 | 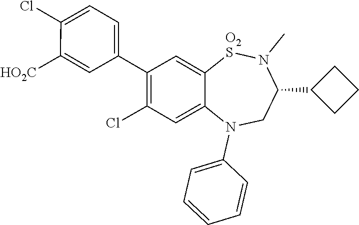 [SMILES: CN1[C@H](C2CCC2)CN(c2ccccc2)c2cc(Cl)c(-c3ccc(Cl)c(C(=O)O)c3)cc2S1(=O)=O] |
| 82 | 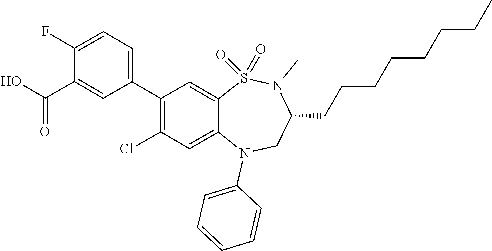 [SMILES: CCCCCCCC[C@@H]1CN(c2ccccc2)c2cc(Cl)c(-c3ccc(F)c(C(=O)O)c3)cc2S(=O)(=O)N1C] |
| 83 | 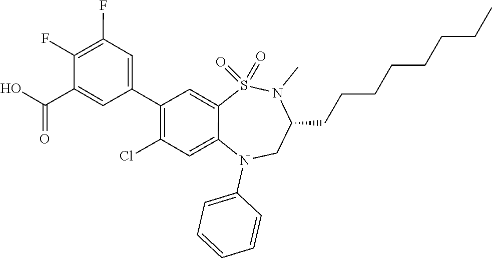 [SMILES: CCCCCCCC[C@@H]1CN(c2ccccc2)c2cc(Cl)c(-c3cc(F)c(F)c(C(=O)O)c3)cc2S(=O)(=O)N1C] |
| 84 | 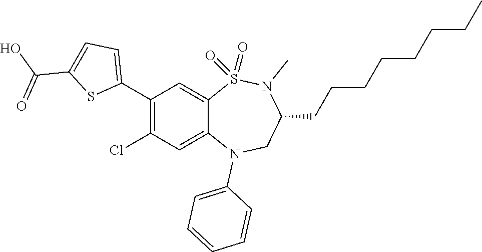 [SMILES: CCCCCCCC[C@@H]1CN(c2ccccc2)c2cc(Cl)c(-c3ccc(C(=O)O)s3)cc2S(=O)(=O)N1C] |
| 85 | 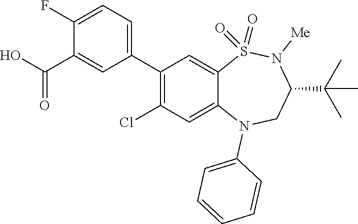 [SMILES: CN1[C@H](C(C)(C)C)CN(c2ccccc2)c2cc(Cl)c(-c3ccc(F)c(C(=O)O)c3)cc2S1(=O)=O] |
| 86 | 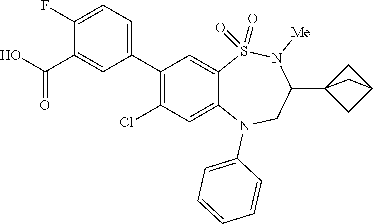 [SMILES: CN1C(C23CC(C2)C3)CN(c2ccccc2)c2cc(Cl)c(-c3ccc(F)c(C(=O)O)c3)cc2S1(=O)=O] |
| 87 | 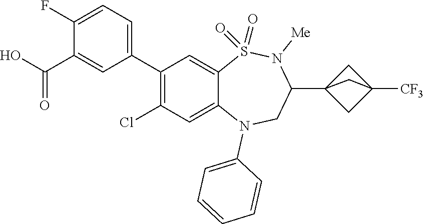 [SMILES: CN1C(C23CC(C(F)(F)F)(C2)C3)CN(c2ccccc2)c2cc(Cl)c(-c3ccc(F)c(C(=O)O)c3)cc2S1(=O)=O] |
| 88 | 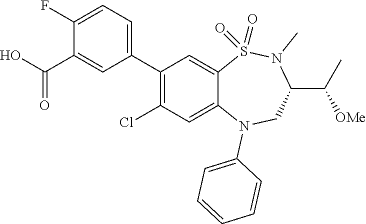 [SMILES: CO[C@@H](C)[C@@H]1CN(c2ccccc2)c2cc(Cl)c(-c3ccc(F)c(C(=O)O)c3)cc2S(=O)(=O)N1C] |
| 89 | 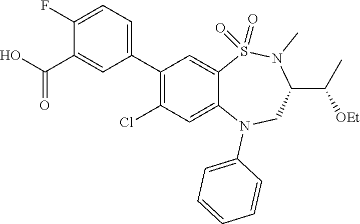 [SMILES: CCO[C@@H](C)[C@@H]1CN(c2ccccc2)c2cc(Cl)c(-c3ccc(F)c(C(=O)O)c3)cc2S(=O)(=O)N1C] |
| 90 | 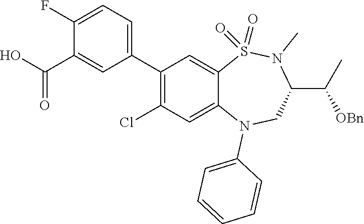 [SMILES: C[C@H](OCc1ccccc1)[C@@H]1CN(c2ccccc2)c2cc(Cl)c(-c3ccc(F)c(C(=O)O)c3)cc2S(=O)(=O)N1C] |
| 91 | 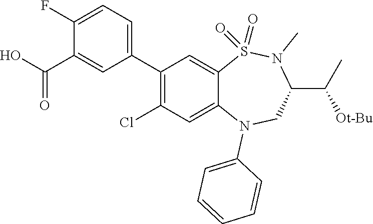 [SMILES: C[C@H](OC(C)(C)C)[C@@H]1CN(c2ccccc2)c2cc(Cl)c(-c3ccc(F)c(C(=O)O)c3)cc2S(=O)(=O)N1C] |
| 92 | 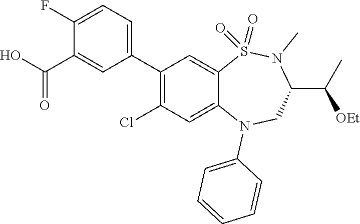 [SMILES: CCO[C@H](C)[C@@H]1CN(c2ccccc2)c2cc(Cl)c(-c3ccc(F)c(C(=O)O)c3)cc2S(=O)(=O)N1C] |
| 93 | 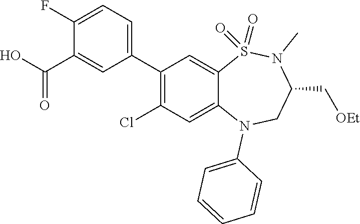 [SMILES: CCOC[C@@H]1CN(c2ccccc2)c2cc(Cl)c(-c3ccc(F)c(C(=O)O)c3)cc2S(=O)(=O)N1C] |
| 94 | 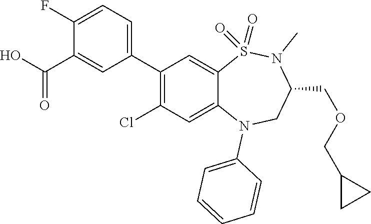 [SMILES: CN1[C@H](COCC2CC2)CN(c2ccccc2)c2cc(Cl)c(-c3ccc(F)c(C(=O)O)c3)cc2S1(=O)=O] |
| 95 | 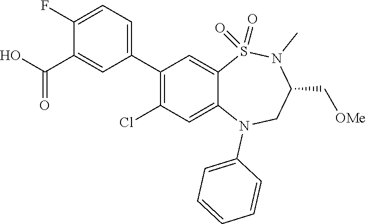 [SMILES: COC[C@@H]1CN(c2ccccc2)c2cc(Cl)c(-c3ccc(F)c(C(=O)O)c3)cc2S(=O)(=O)N1C] |
| 96 | 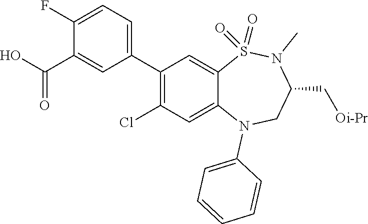 |
| 97 | 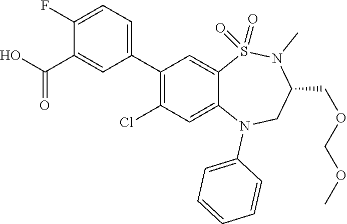 [SMILES: COCOC[C@@H]1CN(c2ccccc2)c2cc(Cl)c(-c3ccc(F)c(C(=O)O)c3)cc2S(=O)(=O)N1C] |
| 98 | 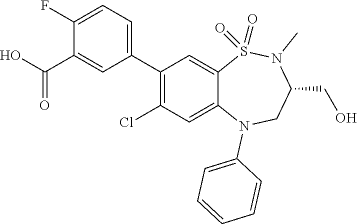 [SMILES: CN1[C@H](CO)CN(c2ccccc2)c2cc(Cl)c(-c3ccc(F)c(C(=O)O)c3)cc2S1(=O)=O] |
| 99 | 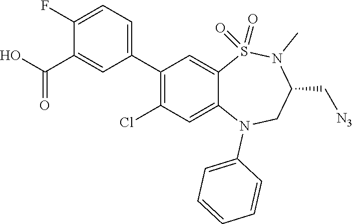 [SMILES: CN1[C@H](CN=[N+]=[N-])CN(c2ccccc2)c2cc(Cl)c(-c3ccc(F)c(C(=O)O)c3)cc2S1(=O)=O] |
| 100 | 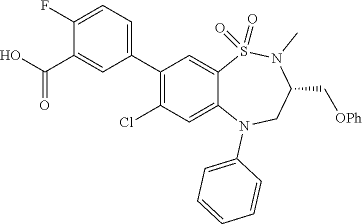 [SMILES: CN1[C@H](COc2ccccc2)CN(c2ccccc2)c2cc(Cl)c(-c3ccc(F)c(C(=O)O)c3)cc2S1(=O)=O] |
| 101 | 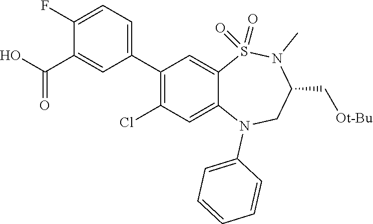 [SMILES: CN1[C@H](COC(C)(C)C)CN(c2ccccc2)c2cc(Cl)c(-c3ccc(F)c(C(=O)O)c3)cc2S1(=O)=O] |
| 102 | 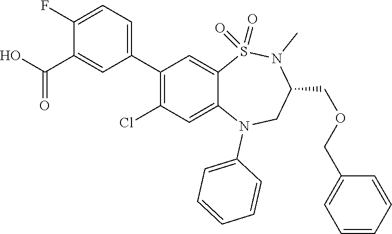 [SMILES: CN1[C@H](COCc2ccccc2)CN(c2ccccc2)c2cc(Cl)c(-c3ccc(F)c(C(=O)O)c3)cc2S1(=O)=O] |
| 103 | 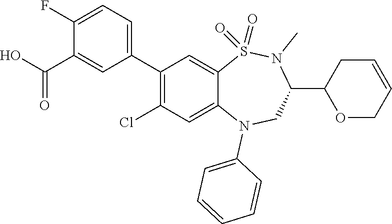 [SMILES: CN1[C@H](C2CC=CCO2)CN(c2ccccc2)c2cc(Cl)c(-c3ccc(F)c(C(=O)O)c3)cc2S1(=O)=O] |
| 104 | 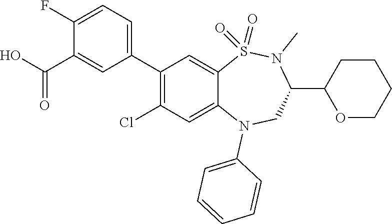 [SMILES: CN1[C@H](C2CCCCO2)CN(c2ccccc2)c2cc(Cl)c(-c3ccc(F)c(C(=O)O)c3)cc2S1(=O)=O] |
| 105 | 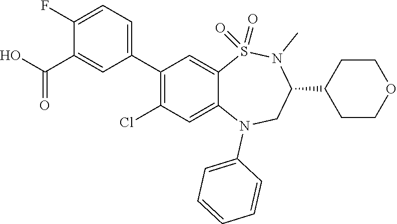 [SMILES: CN1[C@H](C2CCOCC2)CN(c2ccccc2)c2cc(Cl)c(-c3ccc(F)c(C(=O)O)c3)cc2S1(=O)=O] |
| 106 | 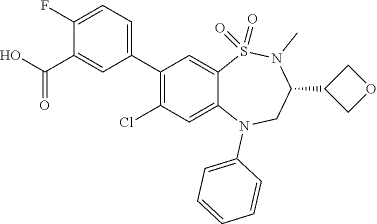 [SMILES: CN1[C@H](C2COC2)CN(c2ccccc2)c2cc(Cl)c(-c3ccc(F)c(C(=O)O)c3)cc2S1(=O)=O] |
| 107 | 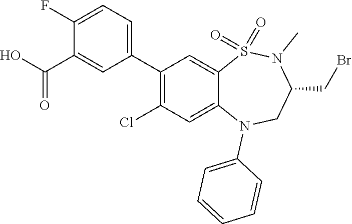 [SMILES: CN1[C@H](CBr)CN(c2ccccc2)c2cc(Cl)c(-c3ccc(F)c(C(=O)O)c3)cc2S1(=O)=O] |
| 108 | 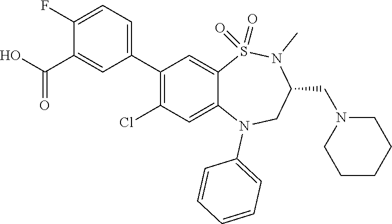 [SMILES: CN1[C@H](CN2CCCCC2)CN(c2ccccc2)c2cc(Cl)c(-c3ccc(F)c(C(=O)O)c3)cc2S1(=O)=O] |
| 109 | 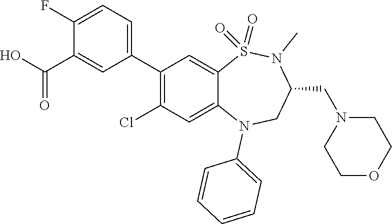 [SMILES: CN1[C@H](CN2CCOCC2)CN(c2ccccc2)c2cc(Cl)c(-c3ccc(F)c(C(=O)O)c3)cc2S1(=O)=O] |
| 110 | 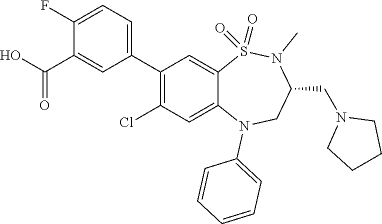 [SMILES: CN1[C@H](CN2CCCC2)CN(c2ccccc2)c2cc(Cl)c(-c3ccc(F)c(C(=O)O)c3)cc2S1(=O)=O] |
| 111 | 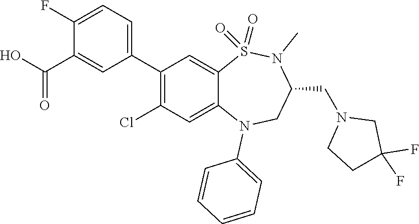 [SMILES: CN1[C@H](CN2CCC(F)(F)C2)CN(c2ccccc2)c2cc(Cl)c(-c3ccc(F)c(C(=O)O)c3)cc2S1(=O)=O] |
| 112 | 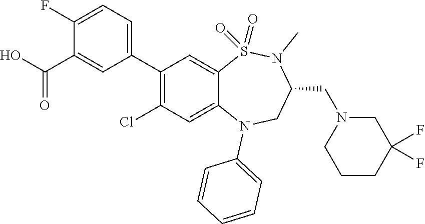 [SMILES: CN1[C@H](CN2CCCC(F)(F)C2)CN(c2ccccc2)c2cc(Cl)c(-c3ccc(F)c(C(=O)O)c3)cc2S1(=O)=O] |
| 113 | 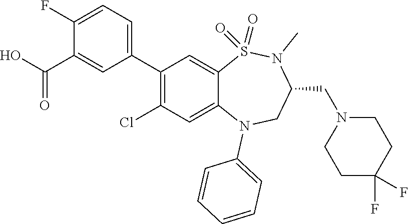 [SMILES: CN1[C@H](CN2CCC(F)(F)CC2)CN(c2ccccc2)c2cc(Cl)c(-c3ccc(F)c(C(=O)O)c3)cc2S1(=O)=O] |
| 114 | 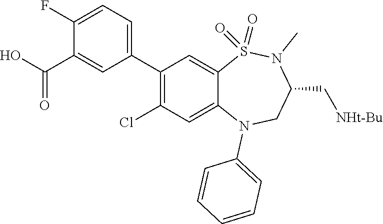 |
| 115 | 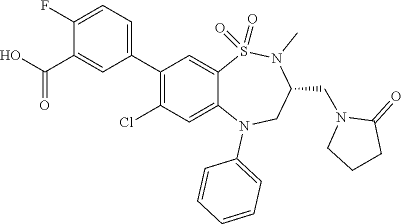 [SMILES: CN1[C@H](CN2CCCC2=O)CN(c2ccccc2)c2cc(Cl)c(-c3ccc(F)c(C(=O)O)c3)cc2S1(=O)=O] |
| 116 | 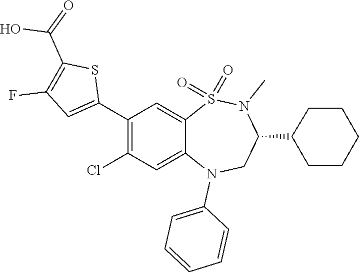 [SMILES: CN1[C@H](C2CCCCC2)CN(c2ccccc2)c2cc(Cl)c(-c3cc(F)c(C(=O)O)s3)cc2S1(=O)=O] |
| 117 | 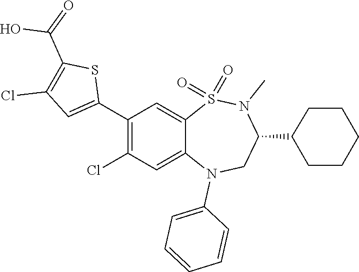 [SMILES: CN1[C@H](C2CCCCC2)CN(c2ccccc2)c2cc(Cl)c(-c3cc(Cl)c(C(=O)O)s3)cc2S1(=O)=O] |
| 118 | 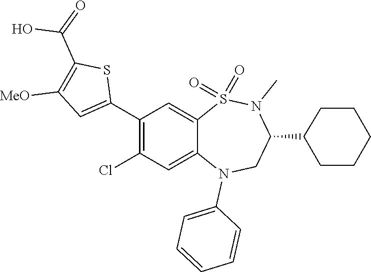 [SMILES: COc1cc(-c2cc3c(cc2Cl)N(c2ccccc2)C[C@@H](C2CCCCC2)N(C)S3(=O)=O)sc1C(=O)O] |
| 119 | 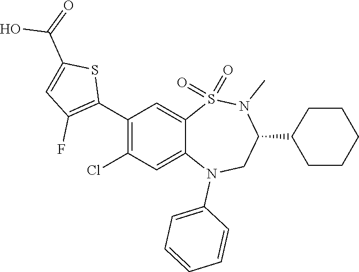 [SMILES: CN1[C@H](C2CCCCC2)CN(c2ccccc2)c2cc(Cl)c(-c3sc(C(=O)O)cc3F)cc2S1(=O)=O] |
| 120 | 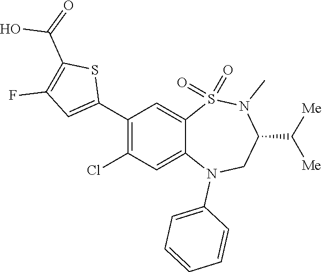 [SMILES: CC(C)[C@@H]1CN(c2ccccc2)c2cc(Cl)c(-c3cc(F)c(C(=O)O)s3)cc2S(=O)(=O)N1C] |
| 121 | 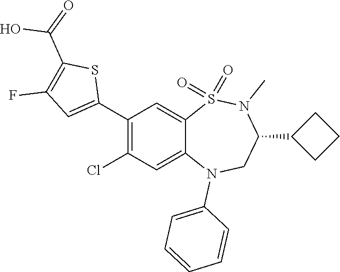 [SMILES: CN1[C@H](C2CCC2)CN(c2ccccc2)c2cc(Cl)c(-c3cc(F)c(C(=O)O)s3)cc2S1(=O)=O] |
| 122 | 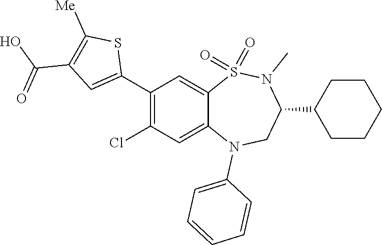 [SMILES: Cc1sc(-c2cc3c(cc2Cl)N(c2ccccc2)C[C@@H](C2CCCCC2)N(C)S3(=O)=O)cc1C(=O)O] |
| 123 | 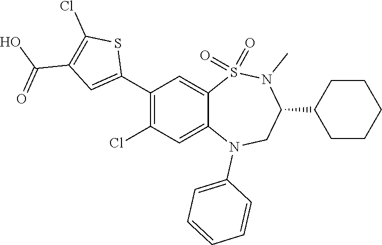 [SMILES: CN1[C@H](C2CCCCC2)CN(c2ccccc2)c2cc(Cl)c(-c3cc(C(=O)O)c(Cl)s3)cc2S1(=O)=O] |
| 124 | 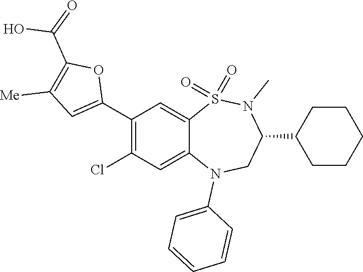 [SMILES: Cc1cc(-c2cc3c(cc2Cl)N(c2ccccc2)C[C@@H](C2CCCCC2)N(C)S3(=O)=O)oc1C(=O)O] |
| 125 | 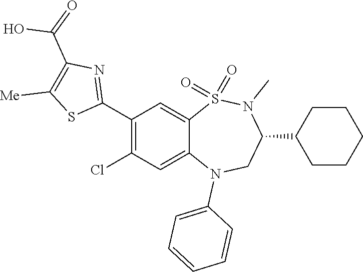 [SMILES: Cc1sc(-c2cc3c(cc2Cl)N(c2ccccc2)C[C@@H](C2CCCCC2)N(C)S3(=O)=O)nc1C(=O)O] |
| 126 | 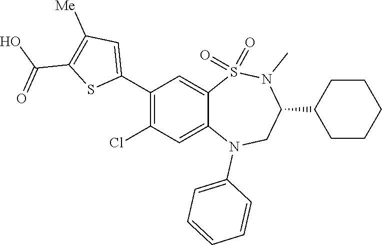 [SMILES: Cc1cc(-c2cc3c(cc2Cl)N(c2ccccc2)C[C@@H](C2CCCCC2)N(C)S3(=O)=O)sc1C(=O)O] |
| 127 | 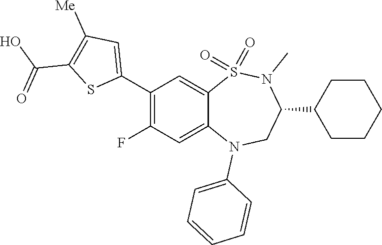 [SMILES: Cc1cc(-c2cc3c(cc2F)N(c2ccccc2)C[C@@H](C2CCCCC2)N(C)S3(=O)=O)sc1C(=O)O] |
| 128 | 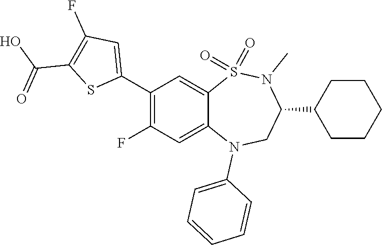 [SMILES: CN1[C@H](C2CCCCC2)CN(c2ccccc2)c2cc(F)c(-c3cc(F)c(C(=O)O)s3)cc2S1(=O)=O] |
| 129 | 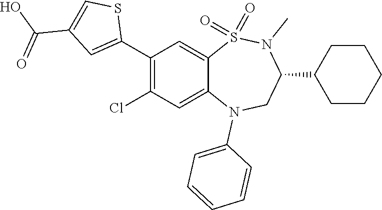 [SMILES: CN1[C@H](C2CCCCC2)CN(c2ccccc2)c2cc(Cl)c(-c3cc(C(=O)O)cs3)cc2S1(=O)=O] |
| 130 | 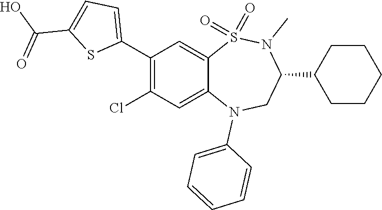 [SMILES: CN1[C@H](C2CCCCC2)CN(c2ccccc2)c2cc(Cl)c(-c3ccc(C(=O)O)s3)cc2S1(=O)=O] |
| 131 | 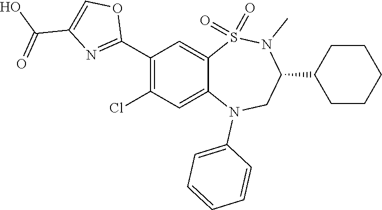 [SMILES: CN1[C@H](C2CCCCC2)CN(c2ccccc2)c2cc(Cl)c(-c3nc(C(=O)O)co3)cc2S1(=O)=O] |
| 132 | 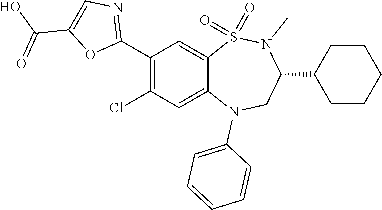 [SMILES: CN1[C@H](C2CCCCC2)CN(c2ccccc2)c2cc(Cl)c(-c3ncc(C(=O)O)o3)cc2S1(=O)=O] |
| 133 | 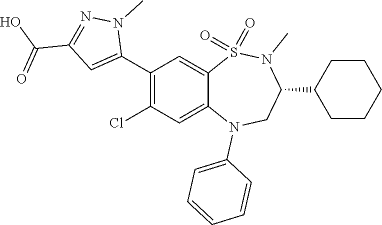 [SMILES: CN1[C@H](C2CCCCC2)CN(c2ccccc2)c2cc(Cl)c(-c3cc(C(=O)O)nn3C)cc2S1(=O)=O] |
| 134 | 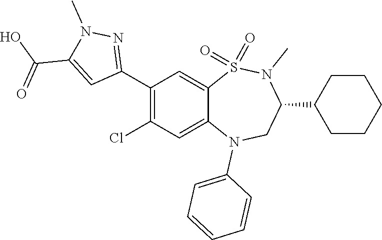 [SMILES: CN1[C@H](C2CCCCC2)CN(c2ccccc2)c2cc(Cl)c(-c3cc(C(=O)O)n(C)n3)cc2S1(=O)=O] |
| 135 | 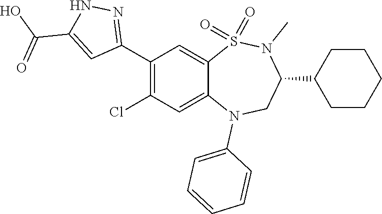 [SMILES: CN1[C@H](C2CCCCC2)CN(c2ccccc2)c2cc(Cl)c(-c3cc(C(=O)O)[nH]n3)cc2S1(=O)=O] |
| 136 | 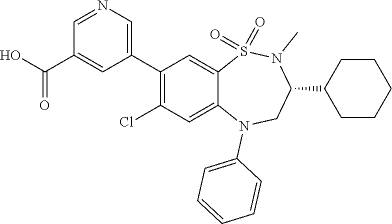 [SMILES: CN1[C@H](C2CCCCC2)CN(c2ccccc2)c2cc(Cl)c(-c3cncc(C(=O)O)c3)cc2S1(=O)=O] |
| 137 | 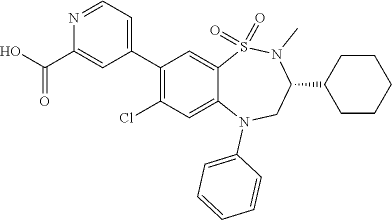 [SMILES: CN1[C@H](C2CCCCC2)CN(c2ccccc2)c2cc(Cl)c(-c3ccnc(C(=O)O)c3)cc2S1(=O)=O] |
| 138 | 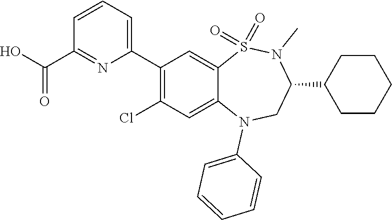 [SMILES: CN1[C@H](C2CCCCC2)CN(c2ccccc2)c2cc(Cl)c(-c3cccc(C(=O)O)n3)cc2S1(=O)=O] |
| 139 | 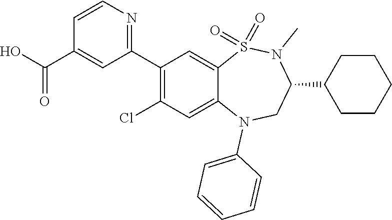 [SMILES: CN1[C@H](C2CCCCC2)CN(c2ccccc2)c2cc(Cl)c(-c3cc(C(=O)O)ccn3)cc2S1(=O)=O] |
| 140 | 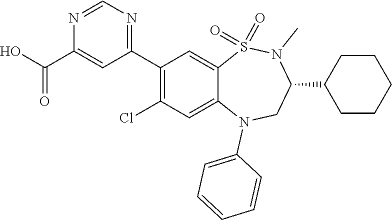 [SMILES: CN1[C@H](C2CCCCC2)CN(c2ccccc2)c2cc(Cl)c(-c3cc(C(=O)O)ncn3)cc2S1(=O)=O] |
| 141 | 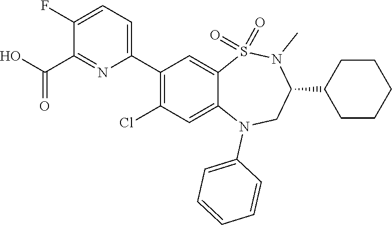 [SMILES: CN1[C@H](C2CCCCC2)CN(c2ccccc2)c2cc(Cl)c(-c3ccc(F)c(C(=O)O)n3)cc2S1(=O)=O] |
| 142 | 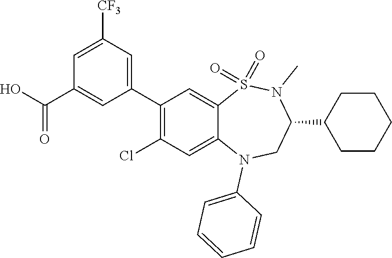 [SMILES: CN1[C@H](C2CCCCC2)CN(c2ccccc2)c2cc(Cl)c(-c3cc(C(=O)O)cc(C(F)(F)F)c3)cc2S1(=O)=O] |
| 143 | 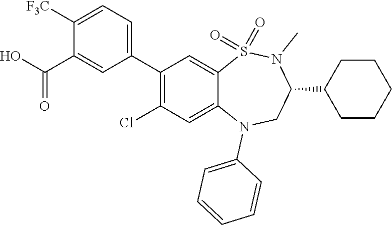 [SMILES: CN1[C@H](C2CCCCC2)CN(c2ccccc2)c2cc(Cl)c(-c3ccc(C(F)(F)F)c(C(=O)O)c3)cc2S1(=O)=O] |
| 144 | 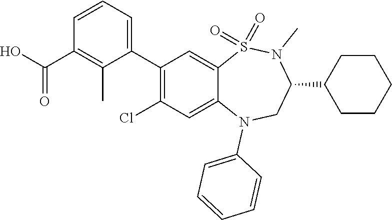 [SMILES: Cc1c(C(=O)O)cccc1-c1cc2c(cc1Cl)N(c1ccccc1)C[C@@H](C1CCCCC1)N(C)S2(=O)=O] |
| 145 | 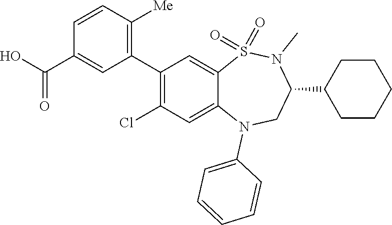 [SMILES: Cc1ccc(C(=O)O)cc1-c1cc2c(cc1Cl)N(c1ccccc1)C[C@@H](C1CCCCC1)N(C)S2(=O)=O] |
| 146 | 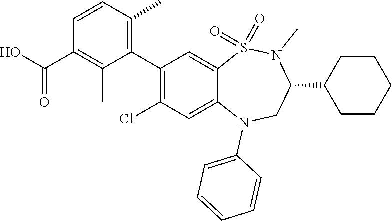 |
| 147 | 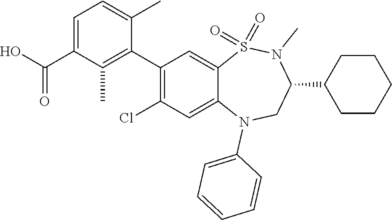 |
| 148 | 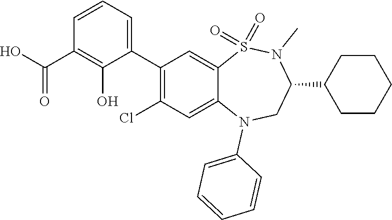 [SMILES: CN1[C@H](C2CCCCC2)CN(c2ccccc2)c2cc(Cl)c(-c3cccc(C(=O)O)c3O)cc2S1(=O)=O] |
| 149 | 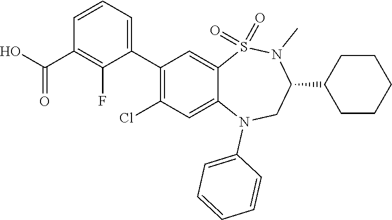 [SMILES: CN1[C@H](C2CCCCC2)CN(c2ccccc2)c2cc(Cl)c(-c3cccc(C(=O)O)c3F)cc2S1(=O)=O] |
| 150 | 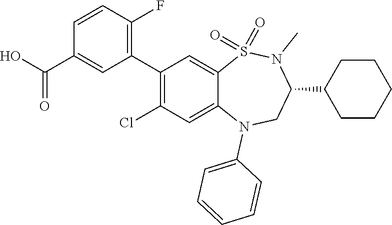 [SMILES: CN1[C@H](C2CCCCC2)CN(c2ccccc2)c2cc(Cl)c(-c3cc(C(=O)O)ccc3F)cc2S1(=O)=O] |
| 151 | 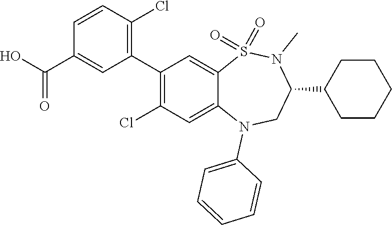 [SMILES: CN1[C@H](C2CCCCC2)CN(c2ccccc2)c2cc(Cl)c(-c3cc(C(=O)O)ccc3Cl)cc2S1(=O)=O] |
| 152 | 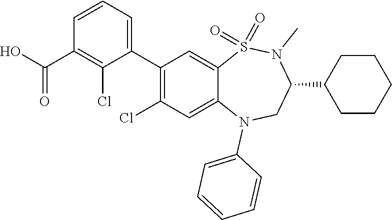 [SMILES: CN1[C@H](C2CCCCC2)CN(c2ccccc2)c2cc(Cl)c(-c3cccc(C(=O)O)c3Cl)cc2S1(=O)=O] |
| 153 | 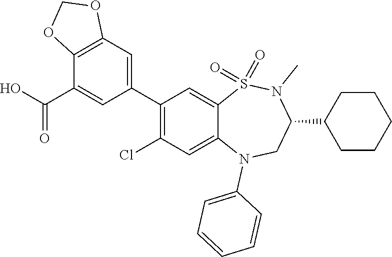 [SMILES: CN1[C@H](C2CCCCC2)CN(c2ccccc2)c2cc(Cl)c(-c3cc4c(c(C(=O)O)c3)OCO4)cc2S1(=O)=O] |
| 154 | 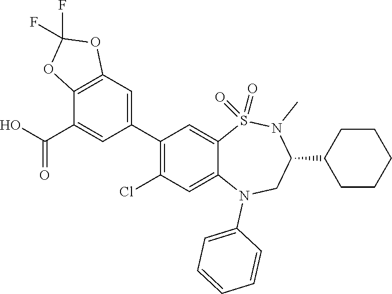 [SMILES: CN1[C@H](C2CCCCC2)CN(c2ccccc2)c2cc(Cl)c(-c3cc4c(c(C(=O)O)c3)OC(F)(F)O4)cc2S1(=O)=O] |
| 155 | 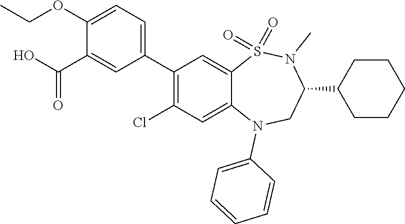 [SMILES: CCOc1ccc(-c2cc3c(cc2Cl)N(c2ccccc2)C[C@@H](C2CCCCC2)N(C)S3(=O)=O)cc1C(=O)O] |
| 156 | 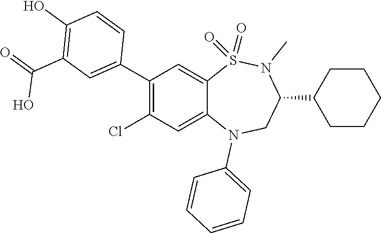 [SMILES: CN1[C@H](C2CCCCC2)CN(c2ccccc2)c2cc(Cl)c(-c3ccc(O)c(C(=O)O)c3)cc2S1(=O)=O] |
| 157 | 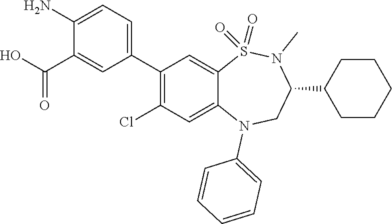 [SMILES: CN1[C@H](C2CCCCC2)CN(c2ccccc2)c2cc(Cl)c(-c3ccc(N)c(C(=O)O)c3)cc2S1(=O)=O] |
| 158 | 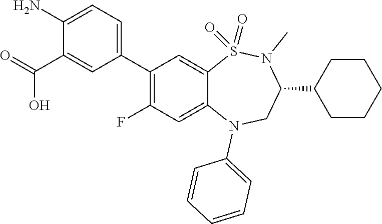 [SMILES: CN1[C@H](C2CCCCC2)CN(c2ccccc2)c2cc(F)c(-c3ccc(N)c(C(=O)O)c3)cc2S1(=O)=O] |
| 159 | 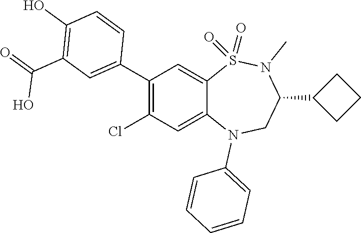 [SMILES: CN1[C@H](C2CCC2)CN(c2ccccc2)c2cc(Cl)c(-c3ccc(O)c(C(=O)O)c3)cc2S1(=O)=O] |
| 160 | 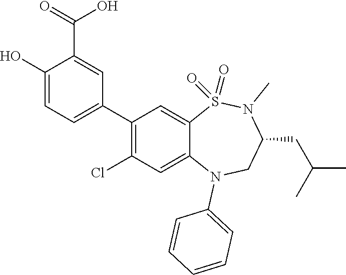 [SMILES: CC(C)C[C@@H]1CN(c2ccccc2)c2cc(Cl)c(-c3ccc(O)c(C(=O)O)c3)cc2S(=O)(=O)N1C] |
| 161 | 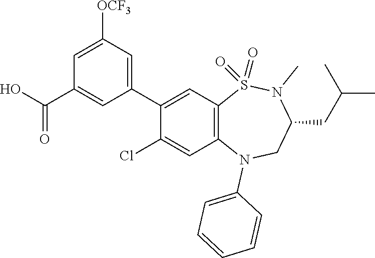 [SMILES: CC(C)C[C@@H]1CN(c2ccccc2)c2cc(Cl)c(-c3cc(OC(F)(F)F)cc(C(=O)O)c3)cc2S(=O)(=O)N1C] |
| 162 | 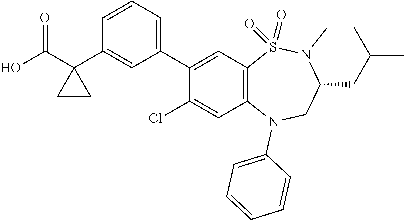 [SMILES: CC(C)C[C@@H]1CN(c2ccccc2)c2cc(Cl)c(-c3cccc(C4(C(=O)O)CC4)c3)cc2S(=O)(=O)N1C] |
| 163 | 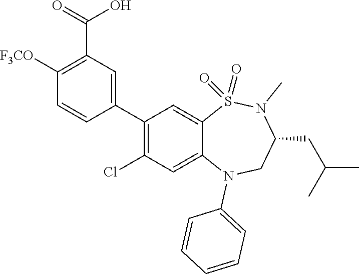 [SMILES: CC(C)C[C@@H]1CN(c2ccccc2)c2cc(Cl)c(-c3ccc(OC(F)(F)F)c(C(=O)O)c3)cc2S(=O)(=O)N1C] |
| 164 | 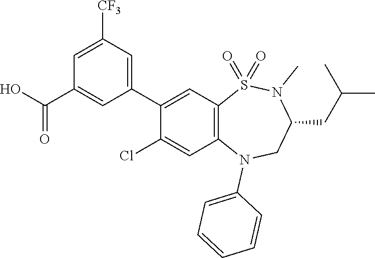 [SMILES: CC(C)C[C@@H]1CN(c2ccccc2)c2cc(Cl)c(-c3cc(C(=O)O)cc(C(F)(F)F)c3)cc2S(=O)(=O)N1C] |
| 165 | 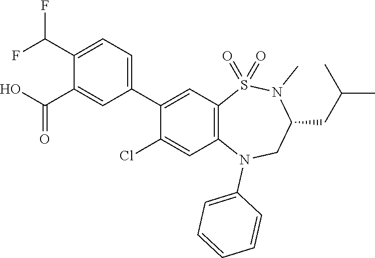 [SMILES: CC(C)C[C@@H]1CN(c2ccccc2)c2cc(Cl)c(-c3ccc(C(F)F)c(C(=O)O)c3)cc2S(=O)(=O)N1C] |
| 166 | 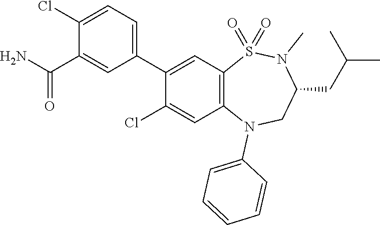 [SMILES: CC(C)C[C@@H]1CN(c2ccccc2)c2cc(Cl)c(-c3ccc(Cl)c(C(N)=O)c3)cc2S(=O)(=O)N1C] |
| 167 | 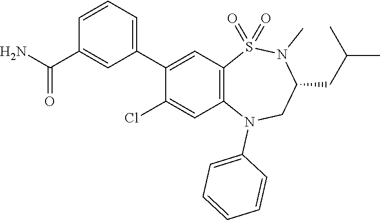 [SMILES: CC(C)C[C@@H]1CN(c2ccccc2)c2cc(Cl)c(-c3cccc(C(N)=O)c3)cc2S(=O)(=O)N1C] |
| 168 | 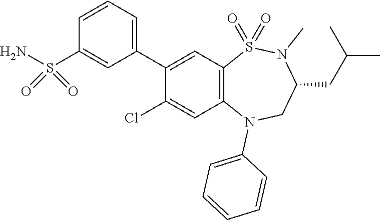 [SMILES: CC(C)C[C@@H]1CN(c2ccccc2)c2cc(Cl)c(-c3cccc(S(N)(=O)=O)c3)cc2S(=O)(=O)N1C] |
| 169 | 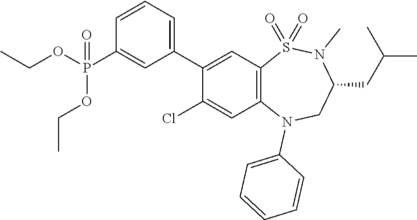 [SMILES: CCOP(=O)(OCC)c1cccc(-c2cc3c(cc2Cl)N(c2ccccc2)C[C@@H](CC(C)C)N(C)S3(=O)=O)c1] |
| 170 | 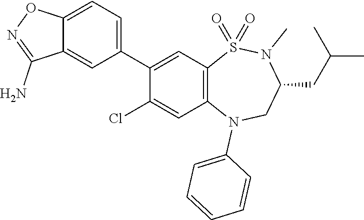 [SMILES: CC(C)C[C@@H]1CN(c2ccccc2)c2cc(Cl)c(-c3ccc4onc(N)c4c3)cc2S(=O)(=O)N1C] |
| 171 | 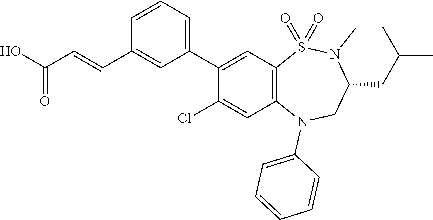 [SMILES: CC(C)C[C@@H]1CN(c2ccccc2)c2cc(Cl)c(-c3cccc(/C=C/C(=O)O)c3)cc2S(=O)(=O)N1C] |
| 172 | 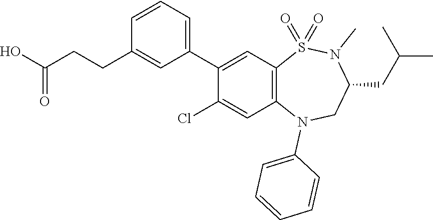 [SMILES: CC(C)C[C@@H]1CN(c2ccccc2)c2cc(Cl)c(-c3cccc(CCC(=O)O)c3)cc2S(=O)(=O)N1C] |
| 173 | 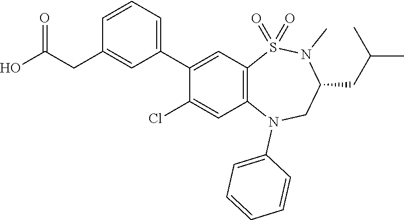 [SMILES: CC(C)C[C@@H]1CN(c2ccccc2)c2cc(Cl)c(-c3cccc(CC(=O)O)c3)cc2S(=O)(=O)N1C] |
| 174 | 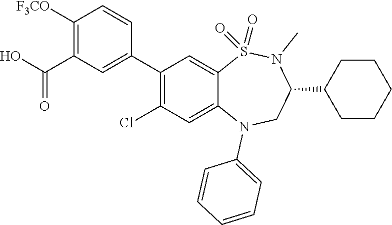 [SMILES: CN1[C@H](C2CCCCC2)CN(c2ccccc2)c2cc(Cl)c(-c3ccc(OC(F)(F)F)c(C(=O)O)c3)cc2S1(=O)=O] |
| 175 | 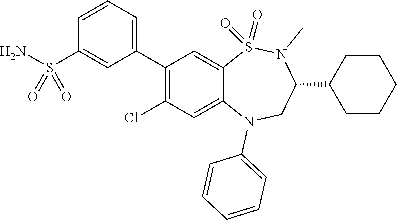 [SMILES: CN1[C@H](C2CCCCC2)CN(c2ccccc2)c2cc(Cl)c(-c3cccc(S(N)(=O)=O)c3)cc2S1(=O)=O] |
| 176 | 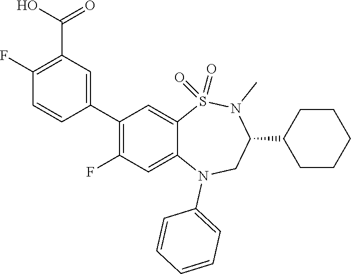 [SMILES: CN1[C@H](C2CCCCC2)CN(c2ccccc2)c2cc(F)c(-c3ccc(F)c(C(=O)O)c3)cc2S1(=O)=O] |
| 177 | 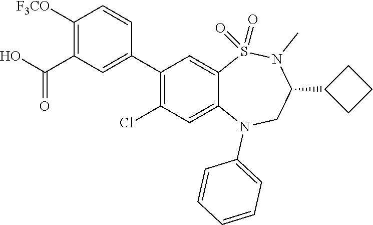 [SMILES: CN1[C@H](C2CCC2)CN(c2ccccc2)c2cc(Cl)c(-c3ccc(OC(F)(F)F)c(C(=O)O)c3)cc2S1(=O)=O] |
| 178 | 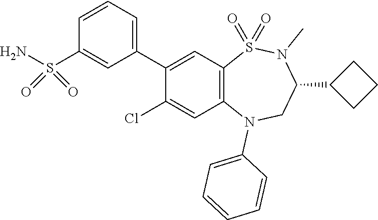 [SMILES: CN1[C@H](C2CCC2)CN(c2ccccc2)c2cc(Cl)c(-c3cccc(S(N)(=O)=O)c3)cc2S1(=O)=O] |
| 179 | 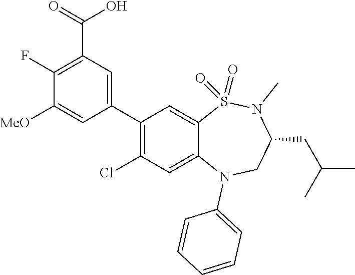 [SMILES: COc1cc(-c2cc3c(cc2Cl)N(c2ccccc2)C[C@@H](CC(C)C)N(C)S3(=O)=O)cc(C(=O)O)c1F] |
| 180 | 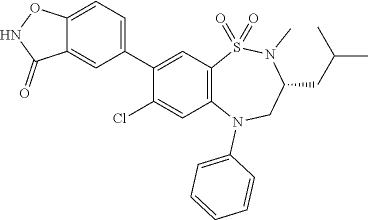 [SMILES: CC(C)C[C@@H]1CN(c2ccccc2)c2cc(Cl)c(-c3ccc4o[nH]c(=O)c4c3)cc2S(=O)(=O)N1C] |
| 181 | 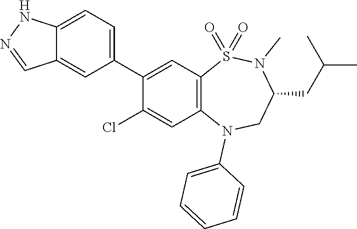 [SMILES: CC(C)C[C@@H]1CN(c2ccccc2)c2cc(Cl)c(-c3ccc4[nH]ncc4c3)cc2S(=O)(=O)N1C] |
| 182 | 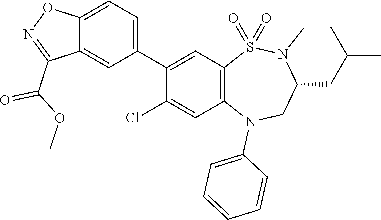 [SMILES: COC(=O)c1noc2ccc(-c3cc4c(cc3Cl)N(c3ccccc3)C[C@@H](CC(C)C)N(C)S4(=O)=O)cc12] |
| 183 | 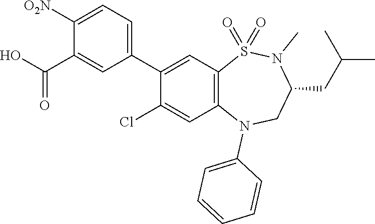 [SMILES: CC(C)C[C@@H]1CN(c2ccccc2)c2cc(Cl)c(-c3ccc([N+](=O)[O-])c(C(=O)O)c3)cc2S(=O)(=O)N1C] |
| 184 | 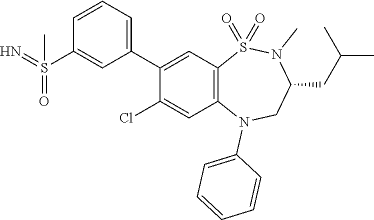 [SMILES: CC(C)C[C@@H]1CN(c2ccccc2)c2cc(Cl)c(-c3cccc(S(C)(=N)=O)c3)cc2S(=O)(=O)N1C] |
| 185 | 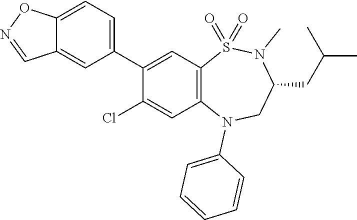 [SMILES: CC(C)C[C@@H]1CN(c2ccccc2)c2cc(Cl)c(-c3ccc4oncc4c3)cc2S(=O)(=O)N1C] |
| 186 | 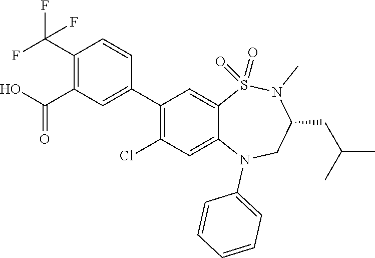 [SMILES: CC(C)C[C@@H]1CN(c2ccccc2)c2cc(Cl)c(-c3ccc(C(F)(F)F)c(C(=O)O)c3)cc2S(=O)(=O)N1C] |
| 187 | 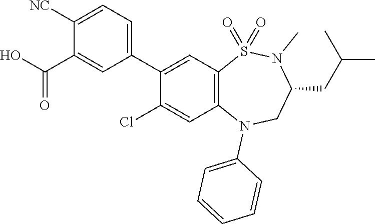 [SMILES: CC(C)C[C@@H]1CN(c2ccccc2)c2cc(Cl)c(-c3ccc(C#N)c(C(=O)O)c3)cc2S(=O)(=O)N1C] |
| 188 | 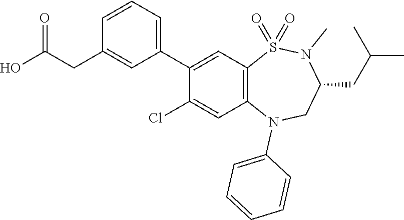 [SMILES: CC(C)C[C@@H]1CN(c2ccccc2)c2cc(Cl)c(-c3cccc(CC(=O)O)c3)cc2S(=O)(=O)N1C] |
| 189 | 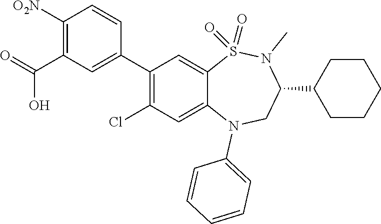 [SMILES: CN1[C@H](C2CCCCC2)CN(c2ccccc2)c2cc(Cl)c(-c3ccc([N+](=O)[O-])c(C(=O)O)c3)cc2S1(=O)=O] |
| 190 | 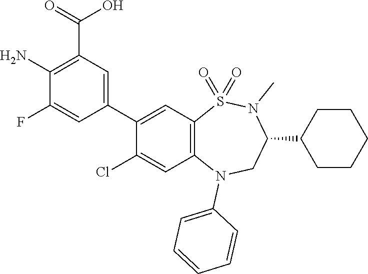 [SMILES: CN1[C@H](C2CCCCC2)CN(c2ccccc2)c2cc(Cl)c(-c3cc(F)c(N)c(C(=O)O)c3)cc2S1(=O)=O] |
| 191 | 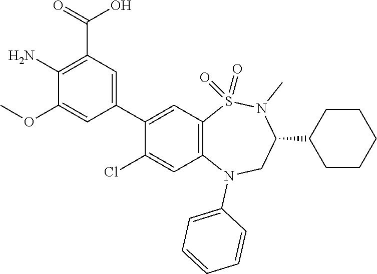 [SMILES: COc1cc(-c2cc3c(cc2Cl)N(c2ccccc2)C[C@@H](C2CCCCC2)N(C)S3(=O)=O)cc(C(=O)O)c1N] |
| 192 | 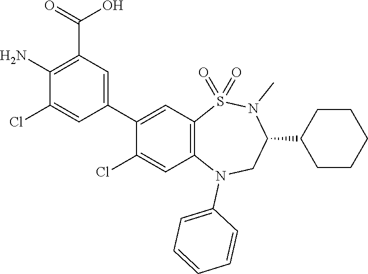 [SMILES: CN1[C@H](C2CCCCC2)CN(c2ccccc2)c2cc(Cl)c(-c3cc(Cl)c(N)c(C(=O)O)c3)cc2S1(=O)=O] |
| 193 | 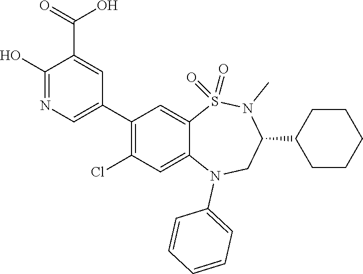 [SMILES: CN1[C@H](C2CCCCC2)CN(c2ccccc2)c2cc(Cl)c(-c3cnc(O)c(C(=O)O)c3)cc2S1(=O)=O] |
| 194 | 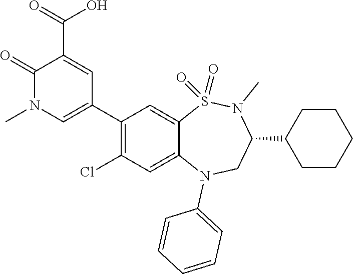 [SMILES: CN1[C@H](C2CCCCC2)CN(c2ccccc2)c2cc(Cl)c(-c3cc(C(=O)O)c(=O)n(C)c3)cc2S1(=O)=O] |
| 195 | 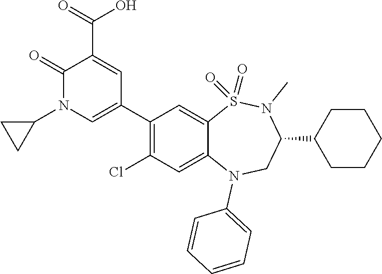 [SMILES: CN1[C@H](C2CCCCC2)CN(c2ccccc2)c2cc(Cl)c(-c3cc(C(=O)O)c(=O)n(C4CC4)c3)cc2S1(=O)=O] |
| 196 | 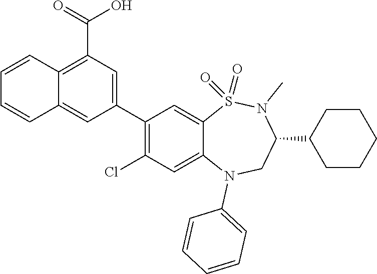 [SMILES: CN1[C@H](C2CCCCC2)CN(c2ccccc2)c2cc(Cl)c(-c3cc(C(=O)O)c4ccccc4c3)cc2S1(=O)=O] |
| 197 | 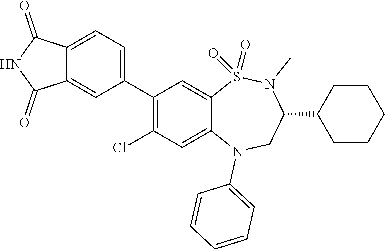 [SMILES: CN1[C@H](C2CCCCC2)CN(c2ccccc2)c2cc(Cl)c(-c3ccc4c(c3)C(=O)NC4=O)cc2S1(=O)=O] |
| 198 | 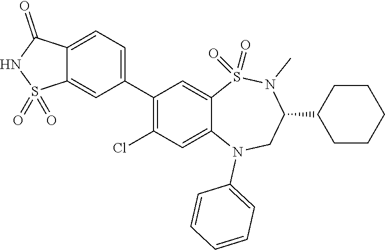 [SMILES: CN1[C@H](C2CCCCC2)CN(c2ccccc2)c2cc(Cl)c(-c3ccc4c(c3)S(=O)(=O)NC4=O)cc2S1(=O)=O] |
| 199 | 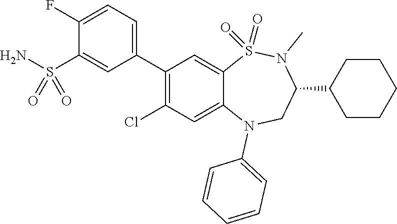 [SMILES: CN1[C@H](C2CCCCC2)CN(c2ccccc2)c2cc(Cl)c(-c3ccc(F)c(S(N)(=O)=O)c3)cc2S1(=O)=O] |
| 200 | 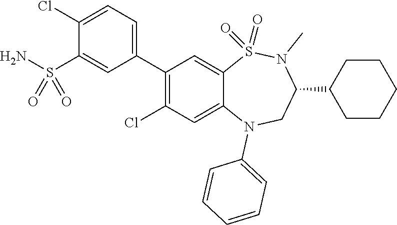 [SMILES: CN1[C@H](C2CCCCC2)CN(c2ccccc2)c2cc(Cl)c(-c3ccc(Cl)c(S(N)(=O)=O)c3)cc2S1(=O)=O] |
| 201 | 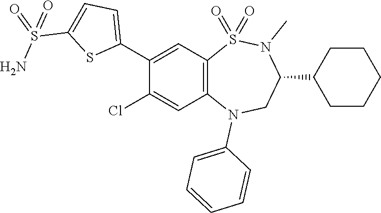 [SMILES: CN1[C@H](C2CCCCC2)CN(c2ccccc2)c2cc(Cl)c(-c3ccc(S(N)(=O)=O)s3)cc2S1(=O)=O] |
| 202 | 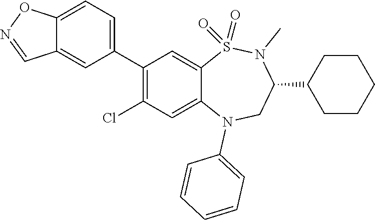 [SMILES: CN1[C@H](C2CCCCC2)CN(c2ccccc2)c2cc(Cl)c(-c3ccc4oncc4c3)cc2S1(=O)=O] |
| 203 | 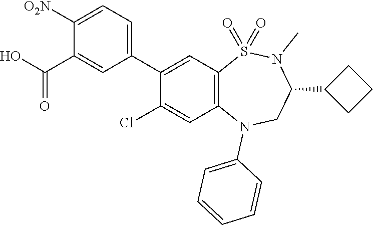 [SMILES: CN1[C@H](C2CCC2)CN(c2ccccc2)c2cc(Cl)c(-c3ccc([N+](=O)[O-])c(C(=O)O)c3)cc2S1(=O)=O] |
| 204 | 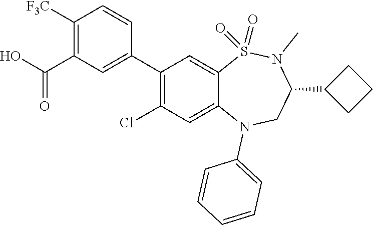 [SMILES: CN1[C@H](C2CCC2)CN(c2ccccc2)c2cc(Cl)c(-c3ccc(C(F)(F)F)c(C(=O)O)c3)cc2S1(=O)=O] |
| 205 | 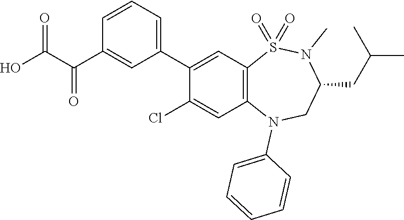 [SMILES: CC(C)C[C@@H]1CN(c2ccccc2)c2cc(Cl)c(-c3cccc(C(=O)C(=O)O)c3)cc2S(=O)(=O)N1C] |
| 206 | 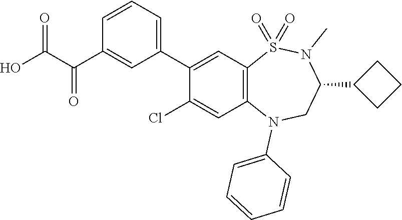 [SMILES: CN1[C@H](C2CCC2)CN(c2ccccc2)c2cc(Cl)c(-c3cccc(C(=O)C(=O)O)c3)cc2S1(=O)=O] |
| 207 | 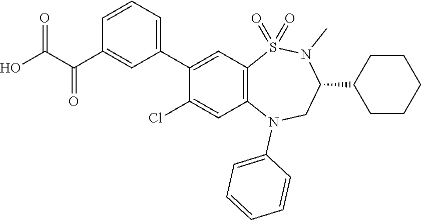 [SMILES: CN1[C@H](C2CCCCC2)CN(c2ccccc2)c2cc(Cl)c(-c3cccc(C(=O)C(=O)O)c3)cc2S1(=O)=O] |
| 208 | 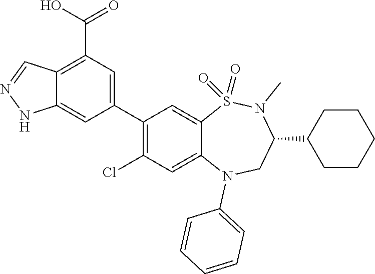 [SMILES: CN1[C@H](C2CCCCC2)CN(c2ccccc2)c2cc(Cl)c(-c3cc(C(=O)O)c4cn[nH]c4c3)cc2S1(=O)=O] |
| 209 | 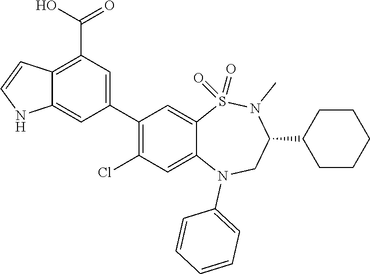 [SMILES: CN1[C@H](C2CCCCC2)CN(c2ccccc2)c2cc(Cl)c(-c3cc(C(=O)O)c4cc[nH]c4c3)cc2S1(=O)=O] |
| 210 | 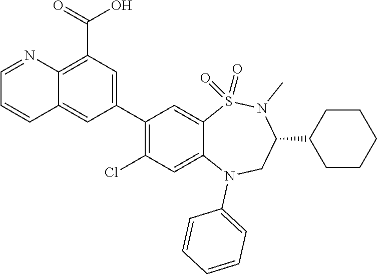 [SMILES: CN1[C@H](C2CCCCC2)CN(c2ccccc2)c2cc(Cl)c(-c3cc(C(=O)O)c4ncccc4c3)cc2S1(=O)=O] |
| 211 | 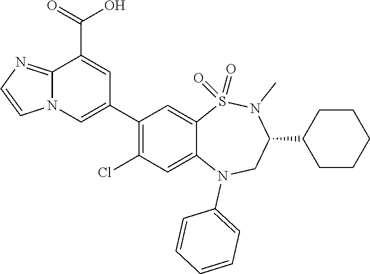 [SMILES: CN1[C@H](C2CCCCC2)CN(c2ccccc2)c2cc(Cl)c(-c3cc(C(=O)O)c4nccn4c3)cc2S1(=O)=O] |
| 212 | 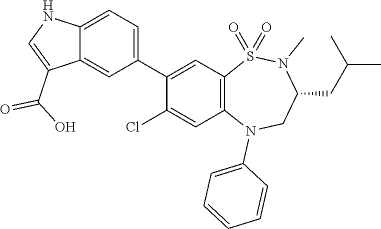 [SMILES: CC(C)C[C@@H]1CN(c2ccccc2)c2cc(Cl)c(-c3ccc4[nH]cc(C(=O)O)c4c3)cc2S(=O)(=O)N1C] |
| 213 | 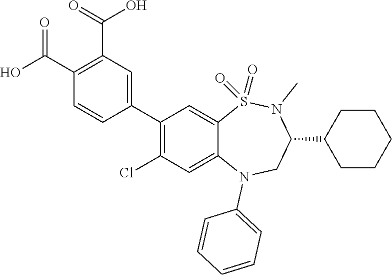 [SMILES: CN1[C@H](C2CCCCC2)CN(c2ccccc2)c2cc(Cl)c(-c3ccc(C(=O)O)c(C(=O)O)c3)cc2S1(=O)=O] |
| 214 | 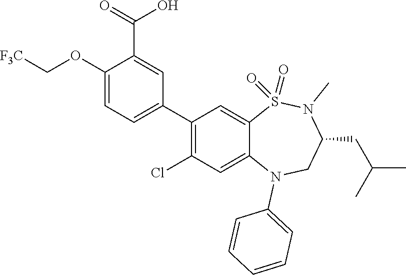 [SMILES: CC(C)C[C@@H]1CN(c2ccccc2)c2cc(Cl)c(-c3ccc(OCC(F)(F)F)c(C(=O)O)c3)cc2S(=O)(=O)N1C] |
| 215 | 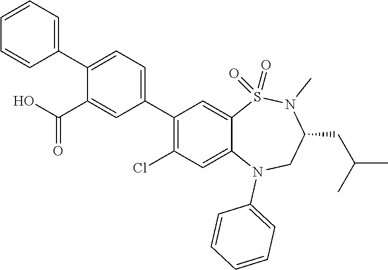 [SMILES: CC(C)C[C@@H]1CN(c2ccccc2)c2cc(Cl)c(-c3ccc(-c4ccccc4)c(C(=O)O)c3)cc2S(=O)(=O)N1C] |
| 216 | 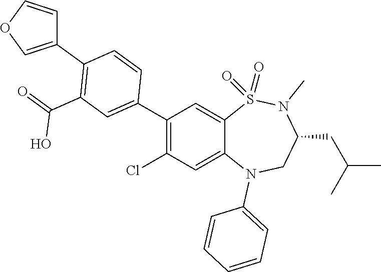 [SMILES: CC(C)C[C@@H]1CN(c2ccccc2)c2cc(Cl)c(-c3ccc(-c4ccoc4)c(C(=O)O)c3)cc2S(=O)(=O)N1C] |
| 217 | 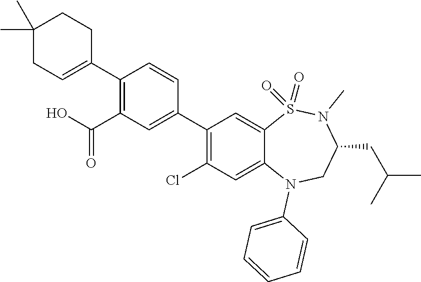 [SMILES: CC(C)C[C@@H]1CN(c2ccccc2)c2cc(Cl)c(-c3ccc(C4=CCC(C)(C)CC4)c(C(=O)O)c3)cc2S(=O)(=O)N1C] |
| 218 | 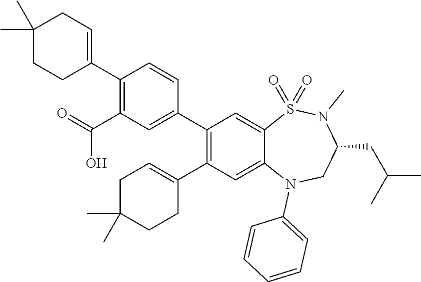 [SMILES: CC(C)C[C@@H]1CN(c2ccccc2)c2cc(C3=CCC(C)(C)CC3)c(-c3ccc(C4=CCC(C)(C)CC4)c(C(=O)O)c3)cc2S(=O)(=O)N1C] |
| 219 | 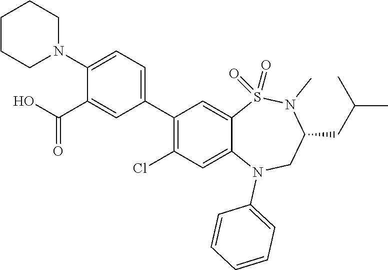 [SMILES: CC(C)C[C@@H]1CN(c2ccccc2)c2cc(Cl)c(-c3ccc(N4CCCCC4)c(C(=O)O)c3)cc2S(=O)(=O)N1C] |
| 220 | 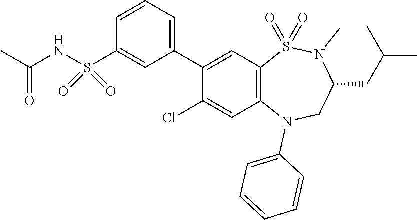 [SMILES: CC(=O)NS(=O)(=O)c1cccc(-c2cc3c(cc2Cl)N(c2ccccc2)C[C@@H](CC(C)C)N(C)S3(=O)=O)c1] |
| 221 | 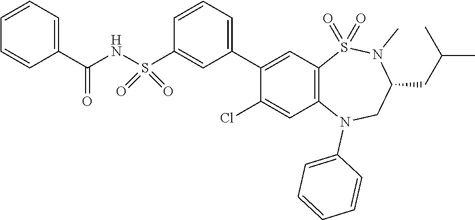 [SMILES: CC(C)C[C@@H]1CN(c2ccccc2)c2cc(Cl)c(-c3cccc(S(=O)(=O)NC(=O)c4ccccc4)c3)cc2S(=O)(=O)N1C] |
| 222 | 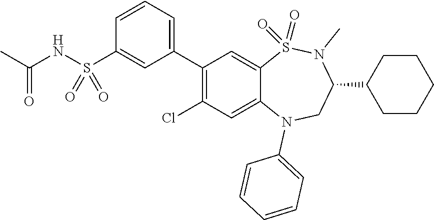 [SMILES: CC(=O)NS(=O)(=O)c1cccc(-c2cc3c(cc2Cl)N(c2ccccc2)C[C@@H](C2CCCCC2)N(C)S3(=O)=O)c1] |
| 223 | 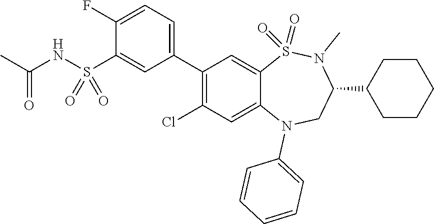 [SMILES: CC(=O)NS(=O)(=O)c1cc(-c2cc3c(cc2Cl)N(c2ccccc2)C[C@@H](C2CCCCC2)N(C)S3(=O)=O)ccc1F] |
| 224 | 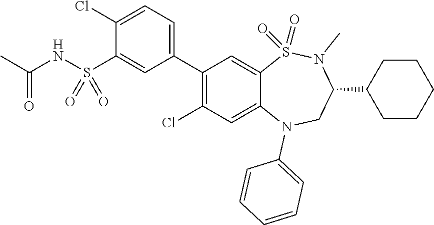 [SMILES: CC(=O)NS(=O)(=O)c1cc(-c2cc3c(cc2Cl)N(c2ccccc2)C[C@@H](C2CCCCC2)N(C)S3(=O)=O)ccc1Cl] |
| 225 | 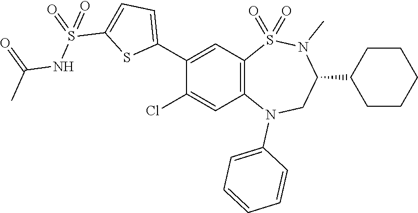 [SMILES: CC(=O)NS(=O)(=O)c1ccc(-c2cc3c(cc2Cl)N(c2ccccc2)C[C@@H](C2CCCCC2)N(C)S3(=O)=O)s1] |
| 226 | 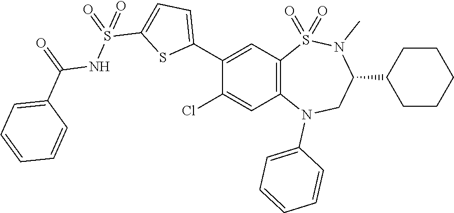 [SMILES: CN1[C@H](C2CCCCC2)CN(c2ccccc2)c2cc(Cl)c(-c3ccc(S(=O)(=O)NC(=O)c4ccccc4)s3)cc2S1(=O)=O] |
| 227 | 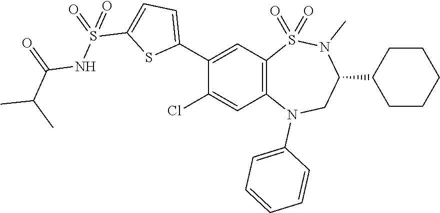 [SMILES: CC(C)C(=O)NS(=O)(=O)c1ccc(-c2cc3c(cc2Cl)N(c2ccccc2)C[C@@H](C2CCCCC2)N(C)S3(=O)=O)s1] |
| 228 | 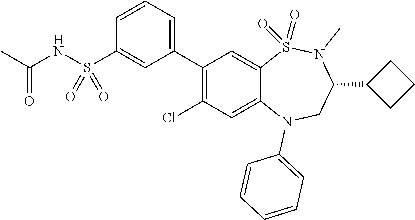 [SMILES: CC(=O)NS(=O)(=O)c1cccc(-c2cc3c(cc2Cl)N(c2ccccc2)C[C@@H](C2CCC2)N(C)S3(=O)=O)c1] |
| 229 | 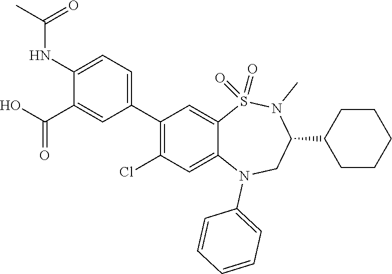 [SMILES: CC(=O)Nc1ccc(-c2cc3c(cc2Cl)N(c2ccccc2)C[C@@H](C2CCCCC2)N(C)S3(=O)=O)cc1C(=O)O] |
| 230 | 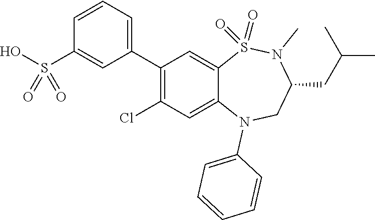 [SMILES: CC(C)C[C@@H]1CN(c2ccccc2)c2cc(Cl)c(-c3cccc(S(=O)(=O)O)c3)cc2S(=O)(=O)N1C] |
| 231 | 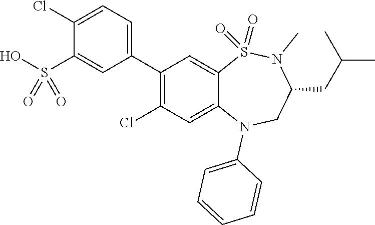 [SMILES: CC(C)C[C@@H]1CN(c2ccccc2)c2cc(Cl)c(-c3ccc(Cl)c(S(=O)(=O)O)c3)cc2S(=O)(=O)N1C] |
| 232 | 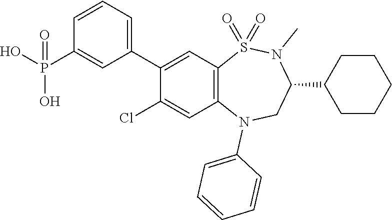 [SMILES: CN1[C@H](C2CCCCC2)CN(c2ccccc2)c2cc(Cl)c(-c3cccc(P(=O)(O)O)c3)cc2S1(=O)=O] |
| 233 | 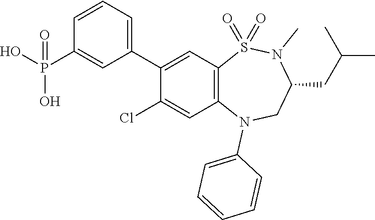 [SMILES: CC(C)C[C@@H]1CN(c2ccccc2)c2cc(Cl)c(-c3cccc(P(=O)(O)O)c3)cc2S(=O)(=O)N1C] |
| 234 | 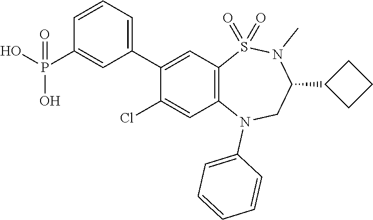 [SMILES: CN1[C@H](C2CCC2)CN(c2ccccc2)c2cc(Cl)c(-c3cccc(P(=O)(O)O)c3)cc2S1(=O)=O] |
| 235 | 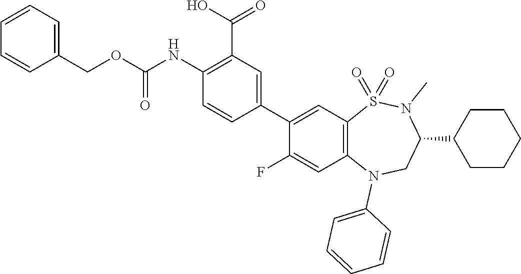 [SMILES: CN1[C@H](C2CCCCC2)CN(c2ccccc2)c2cc(F)c(-c3ccc(NC(=O)OCc4ccccc4)c(C(=O)O)c3)cc2S1(=O)=O] |
| 236 | 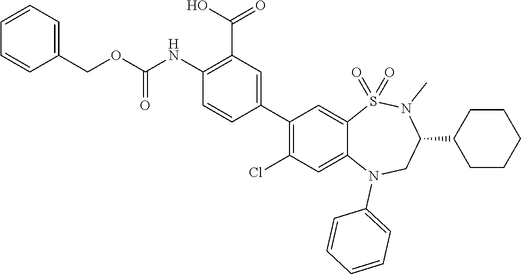 [SMILES: CN1[C@H](C2CCCCC2)CN(c2ccccc2)c2cc(Cl)c(-c3ccc(NC(=O)OCc4ccccc4)c(C(=O)O)c3)cc2S1(=O)=O] |
| 237 | 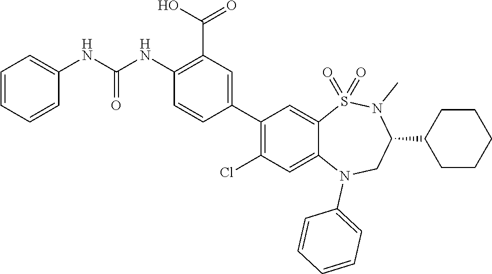 [SMILES: CN1[C@H](C2CCCCC2)CN(c2ccccc2)c2cc(Cl)c(-c3ccc(NC(=O)Nc4ccccc4)c(C(=O)O)c3)cc2S1(=O)=O] |
| 238 | 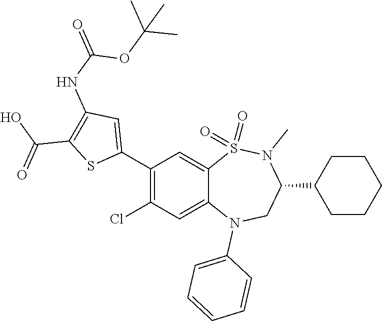 [SMILES: CN1[C@H](C2CCCCC2)CN(c2ccccc2)c2cc(Cl)c(-c3cc(NC(=O)OC(C)(C)C)c(C(=O)O)s3)cc2S1(=O)=O] |
| 239 | 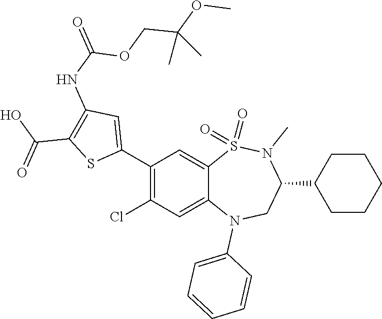 [SMILES: COC(C)(C)COC(=O)Nc1cc(-c2cc3c(cc2Cl)N(c2ccccc2)C[C@@H](C2CCCCC2)N(C)S3(=O)=O)sc1C(=O)O] |
| 240 | 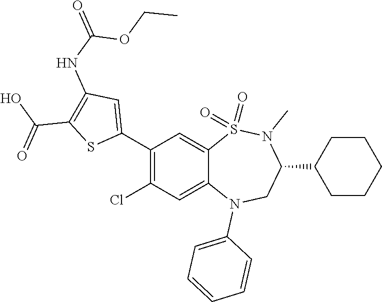 [SMILES: CCOC(=O)Nc1cc(-c2cc3c(cc2Cl)N(c2ccccc2)C[C@@H](C2CCCCC2)N(C)S3(=O)=O)sc1C(=O)O] |
| 241 | 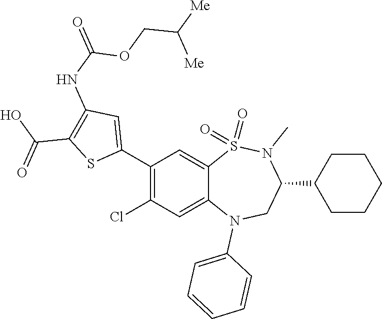 [SMILES: CC(C)COC(=O)Nc1cc(-c2cc3c(cc2Cl)N(c2ccccc2)C[C@@H](C2CCCCC2)N(C)S3(=O)=O)sc1C(=O)O] |
| 242 | 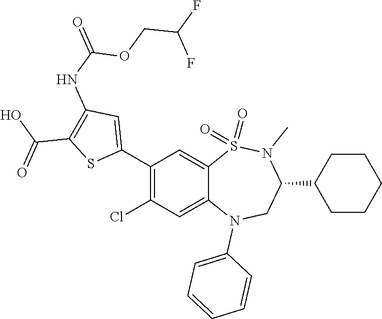 [SMILES: CN1[C@H](C2CCCCC2)CN(c2ccccc2)c2cc(Cl)c(-c3cc(NC(=O)OCC(F)F)c(C(=O)O)s3)cc2S1(=O)=O] |
| 243 | 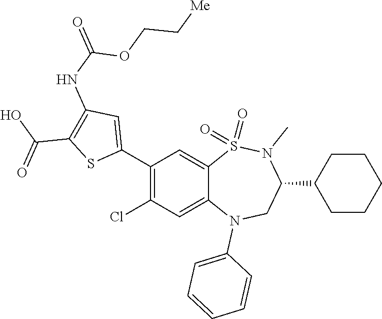 [SMILES: CCCOC(=O)Nc1cc(-c2cc3c(cc2Cl)N(c2ccccc2)C[C@@H](C2CCCCC2)N(C)S3(=O)=O)sc1C(=O)O] |
| 244 | 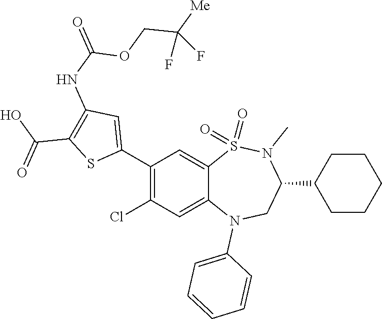 [SMILES: CN1[C@H](C2CCCCC2)CN(c2ccccc2)c2cc(Cl)c(-c3cc(NC(=O)OCC(C)(F)F)c(C(=O)O)s3)cc2S1(=O)=O] |
| 245 | 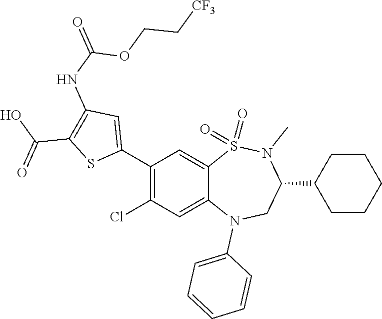 [SMILES: CN1[C@H](C2CCCCC2)CN(c2ccccc2)c2cc(Cl)c(-c3cc(NC(=O)OCCC(F)(F)F)c(C(=O)O)s3)cc2S1(=O)=O] |
| 246 | 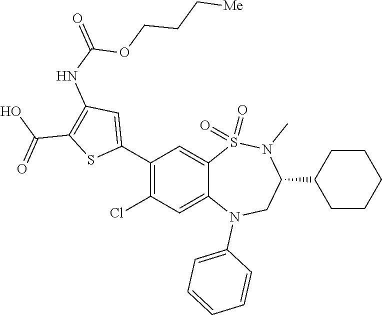 [SMILES: CCCCOC(=O)Nc1cc(-c2cc3c(cc2Cl)N(c2ccccc2)C[C@@H](C2CCCCC2)N(C)S3(=O)=O)sc1C(=O)O] |
| 247 | 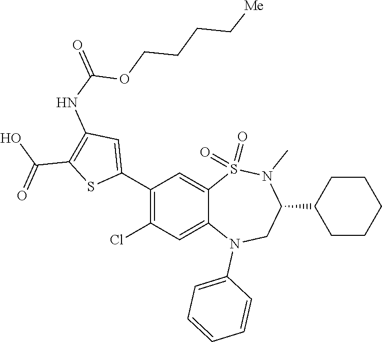 [SMILES: CCCCCOC(=O)Nc1cc(-c2cc3c(cc2Cl)N(c2ccccc2)C[C@@H](C2CCCCC2)N(C)S3(=O)=O)sc1C(=O)O] |
| 248 | 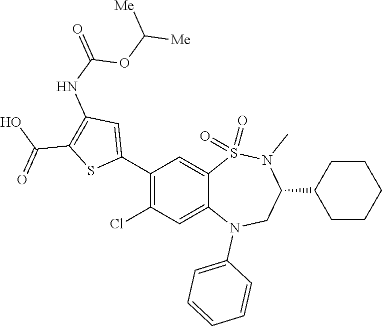 [SMILES: CC(C)OC(=O)Nc1cc(-c2cc3c(cc2Cl)N(c2ccccc2)C[C@@H](C2CCCCC2)N(C)S3(=O)=O)sc1C(=O)O] |
| 249 | 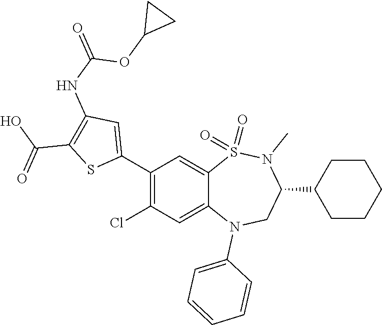 [SMILES: CN1[C@H](C2CCCCC2)CN(c2ccccc2)c2cc(Cl)c(-c3cc(NC(=O)OC4CC4)c(C(=O)O)s3)cc2S1(=O)=O] |
| 250 | 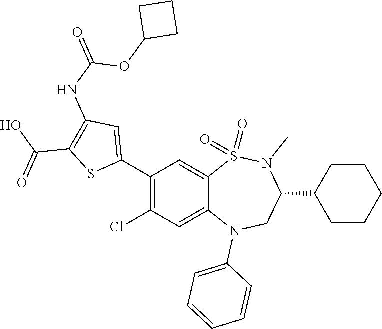 [SMILES: CN1[C@H](C2CCCCC2)CN(c2ccccc2)c2cc(Cl)c(-c3cc(NC(=O)OC4CCC4)c(C(=O)O)s3)cc2S1(=O)=O] |
| 251 | 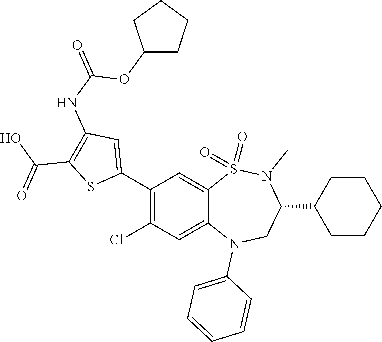 [SMILES: CN1[C@H](C2CCCCC2)CN(c2ccccc2)c2cc(Cl)c(-c3cc(NC(=O)OC4CCCC4)c(C(=O)O)s3)cc2S1(=O)=O] |
| 252 | 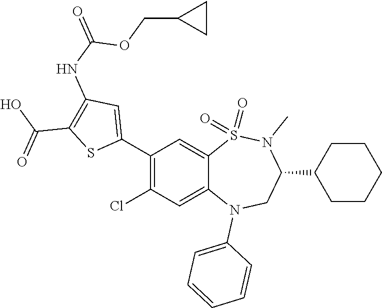 [SMILES: CN1[C@H](C2CCCCC2)CN(c2ccccc2)c2cc(Cl)c(-c3cc(NC(=O)OCC4CC4)c(C(=O)O)s3)cc2S1(=O)=O] |
| 253 | 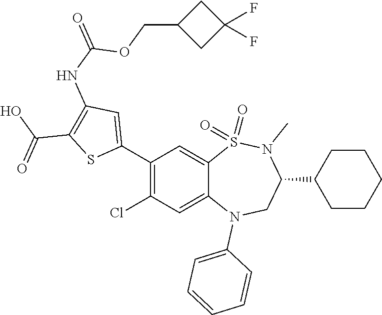 [SMILES: CN1[C@H](C2CCCCC2)CN(c2ccccc2)c2cc(Cl)c(-c3cc(NC(=O)OCC4CC(F)(F)C4)c(C(=O)O)s3)cc2S1(=O)=O] |
| 254 | 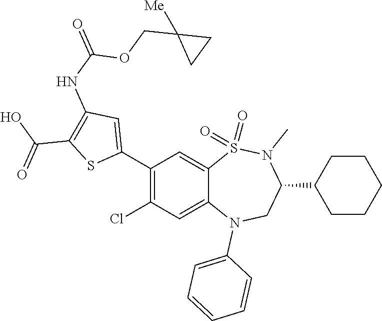 [SMILES: CN1[C@H](C2CCCCC2)CN(c2ccccc2)c2cc(Cl)c(-c3cc(NC(=O)OCC4(C)CC4)c(C(=O)O)s3)cc2S1(=O)=O] |
| 255 | 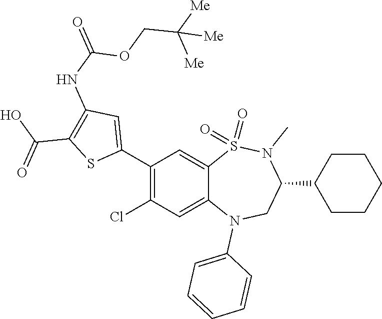 [SMILES: CN1[C@H](C2CCCCC2)CN(c2ccccc2)c2cc(Cl)c(-c3cc(NC(=O)OCC(C)(C)C)c(C(=O)O)s3)cc2S1(=O)=O] |
| 256 | 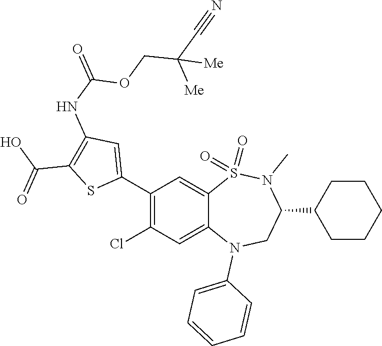 [SMILES: CN1[C@H](C2CCCCC2)CN(c2ccccc2)c2cc(Cl)c(-c3cc(NC(=O)OCC(C)(C)C#N)c(C(=O)O)s3)cc2S1(=O)=O] |
| 257 | 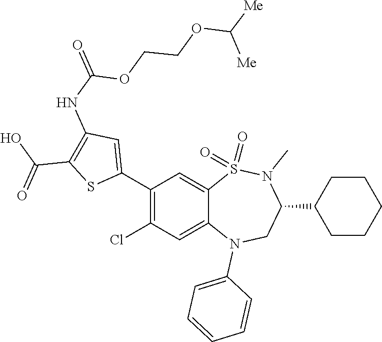 [SMILES: CC(C)OCCOC(=O)Nc1cc(-c2cc3c(cc2Cl)N(c2ccccc2)C[C@@H](C2CCCCC2)N(C)S3(=O)=O)sc1C(=O)O] |
| 258 | 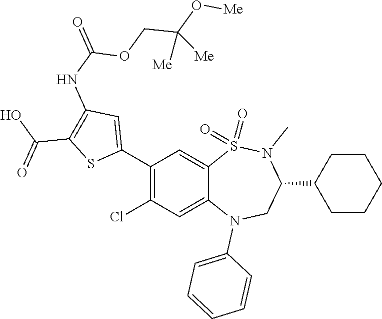 [SMILES: COC(C)(C)COC(=O)Nc1cc(-c2cc3c(cc2Cl)N(c2ccccc2)C[C@@H](C2CCCCC2)N(C)S3(=O)=O)sc1C(=O)O] |
| 259 | 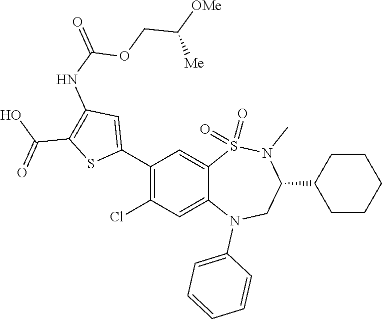 [SMILES: CO[C@H](C)COC(=O)Nc1cc(-c2cc3c(cc2Cl)N(c2ccccc2)C[C@@H](C2CCCCC2)N(C)S3(=O)=O)sc1C(=O)O] |
| 260 | 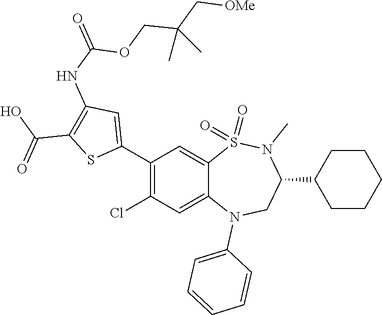 [SMILES: COCC(C)(C)COC(=O)Nc1cc(-c2cc3c(cc2Cl)N(c2ccccc2)C[C@@H](C2CCCCC2)N(C)S3(=O)=O)sc1C(=O)O] |
| 261 | 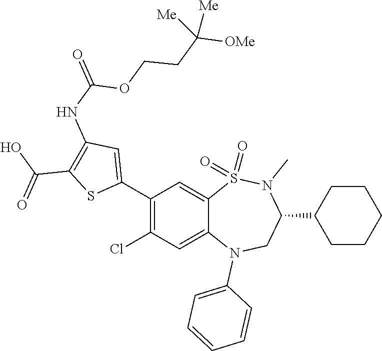 [SMILES: COC(C)(C)CCOC(=O)Nc1cc(-c2cc3c(cc2Cl)N(c2ccccc2)C[C@@H](C2CCCCC2)N(C)S3(=O)=O)sc1C(=O)O] |
| 262 | 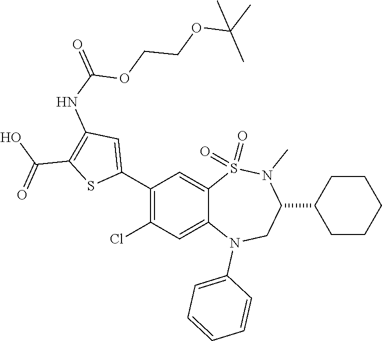 [SMILES: CN1[C@H](C2CCCCC2)CN(c2ccccc2)c2cc(Cl)c(-c3cc(NC(=O)OCCOC(C)(C)C)c(C(=O)O)s3)cc2S1(=O)=O] |
| 263 | 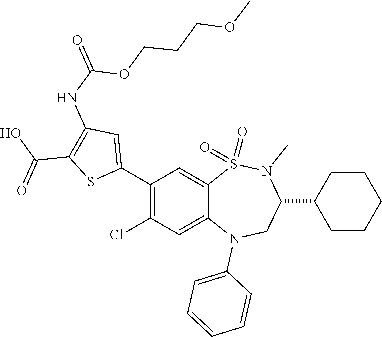 [SMILES: COCCCOC(=O)Nc1cc(-c2cc3c(cc2Cl)N(c2ccccc2)C[C@@H](C2CCCCC2)N(C)S3(=O)=O)sc1C(=O)O] |
| 264 | 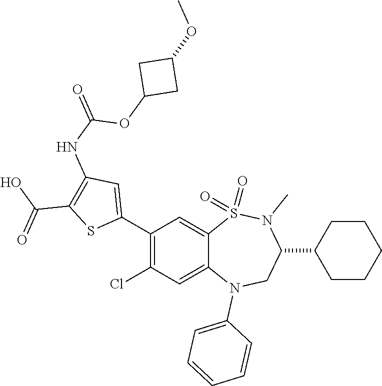 |
| 265 | 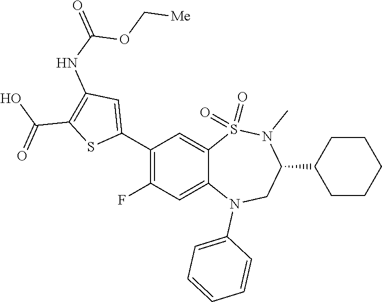 [SMILES: CCOC(=O)Nc1cc(-c2cc3c(cc2F)N(c2ccccc2)C[C@@H](C2CCCCC2)N(C)S3(=O)=O)sc1C(=O)O] |
| 266 | 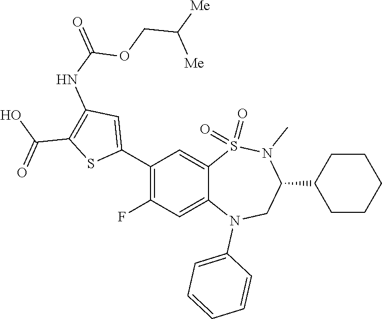 [SMILES: CC(C)COC(=O)Nc1cc(-c2cc3c(cc2F)N(c2ccccc2)C[C@@H](C2CCCCC2)N(C)S3(=O)=O)sc1C(=O)O] |
| 267 | 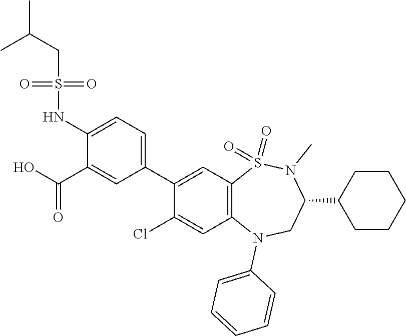 [SMILES: CC(C)CS(=O)(=O)Nc1ccc(-c2cc3c(cc2Cl)N(c2ccccc2)C[C@@H](C2CCCCC2)N(C)S3(=O)=O)cc1C(=O)O] |
| 268 | 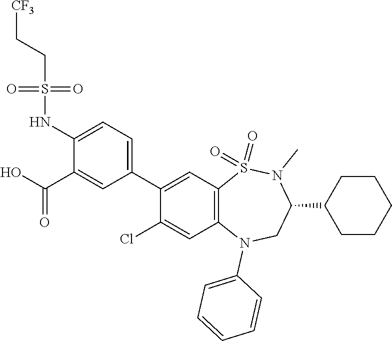 [SMILES: CN1[C@H](C2CCCCC2)CN(c2ccccc2)c2cc(Cl)c(-c3ccc(NS(=O)(=O)CCC(F)(F)F)c(C(=O)O)c3)cc2S1(=O)=O] |
| 269 | 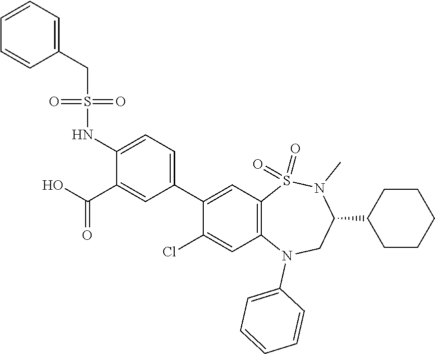 [SMILES: CN1[C@H](C2CCCCC2)CN(c2ccccc2)c2cc(Cl)c(-c3ccc(NS(=O)(=O)Cc4ccccc4)c(C(=O)O)c3)cc2S1(=O)=O] |
| 270 | 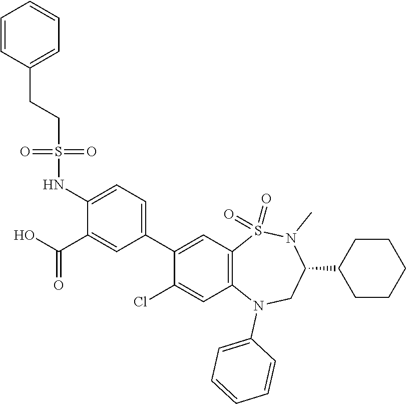 [SMILES: CN1[C@H](C2CCCCC2)CN(c2ccccc2)c2cc(Cl)c(-c3ccc(NS(=O)(=O)CCc4ccccc4)c(C(=O)O)c3)cc2S1(=O)=O] |
| 271 | 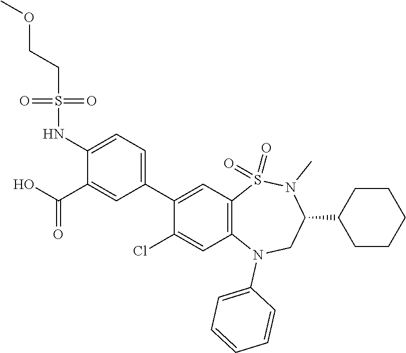 [SMILES: COCCS(=O)(=O)Nc1ccc(-c2cc3c(cc2Cl)N(c2ccccc2)C[C@@H](C2CCCCC2)N(C)S3(=O)=O)cc1C(=O)O] |
| 272 | 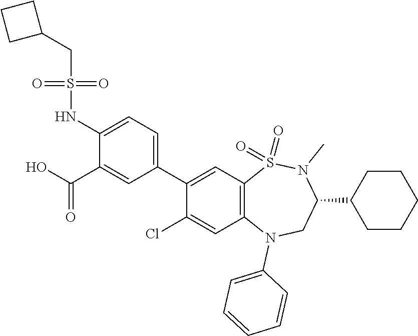 [SMILES: CN1[C@H](C2CCCCC2)CN(c2ccccc2)c2cc(Cl)c(-c3ccc(NS(=O)(=O)CC4CCC4)c(C(=O)O)c3)cc2S1(=O)=O] |
| 273 | 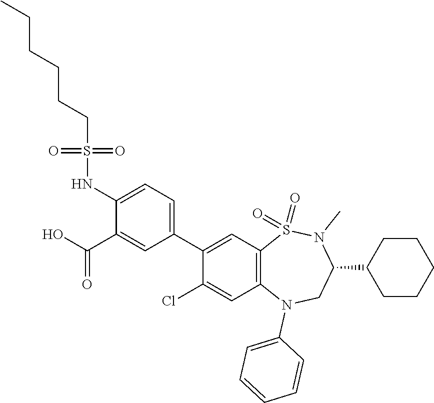 [SMILES: CCCCCCS(=O)(=O)Nc1ccc(-c2cc3c(cc2Cl)N(c2ccccc2)C[C@@H](C2CCCCC2)N(C)S3(=O)=O)cc1C(=O)O] |
| 274 | 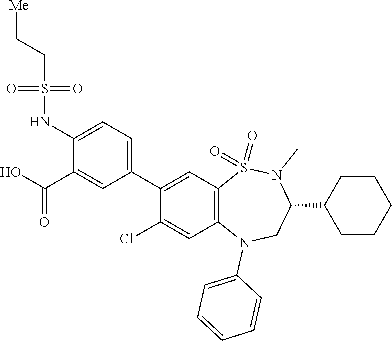 [SMILES: CCCS(=O)(=O)Nc1ccc(-c2cc3c(cc2Cl)N(c2ccccc2)C[C@@H](C2CCCCC2)N(C)S3(=O)=O)cc1C(=O)O] |
| 275 | 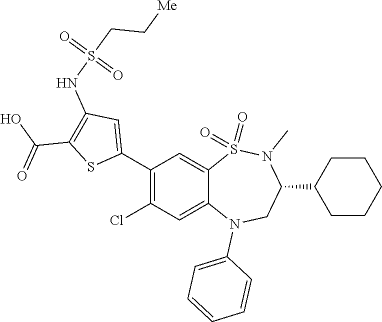 [SMILES: CCCS(=O)(=O)Nc1cc(-c2cc3c(cc2Cl)N(c2ccccc2)C[C@@H](C2CCCCC2)N(C)S3(=O)=O)sc1C(=O)O] |
| 276 | 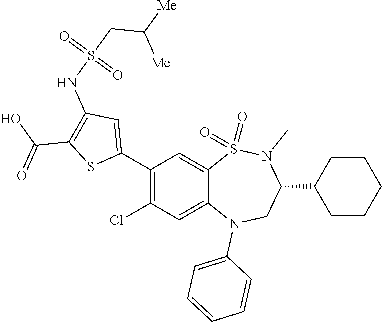 [SMILES: CC(C)CS(=O)(=O)Nc1cc(-c2cc3c(cc2Cl)N(c2ccccc2)C[C@@H](C2CCCCC2)N(C)S3(=O)=O)sc1C(=O)O] |
| 277 | 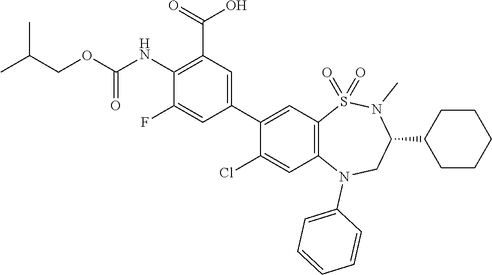 [SMILES: CC(C)COC(=O)Nc1c(F)cc(-c2cc3c(cc2Cl)N(c2ccccc2)C[C@@H](C2CCCCC2)N(C)S3(=O)=O)cc1C(=O)O] |
| 278 | 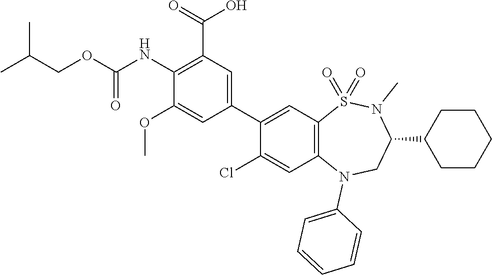 [SMILES: COc1cc(-c2cc3c(cc2Cl)N(c2ccccc2)C[C@@H](C2CCCCC2)N(C)S3(=O)=O)cc(C(=O)O)c1NC(=O)OCC(C)C] |
| 279 | 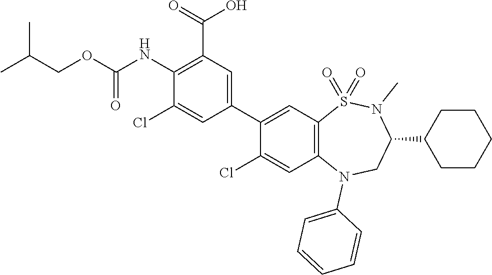 [SMILES: CC(C)COC(=O)Nc1c(Cl)cc(-c2cc3c(cc2Cl)N(c2ccccc2)C[C@@H](C2CCCCC2)N(C)S3(=O)=O)cc1C(=O)O] |
| 280 | 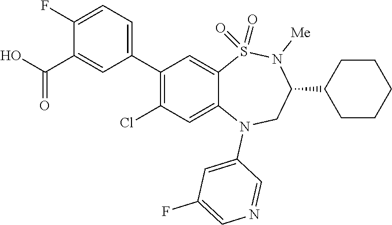 [SMILES: CN1[C@H](C2CCCCC2)CN(c2cncc(F)c2)c2cc(Cl)c(-c3ccc(F)c(C(=O)O)c3)cc2S1(=O)=O] |
| 281 | 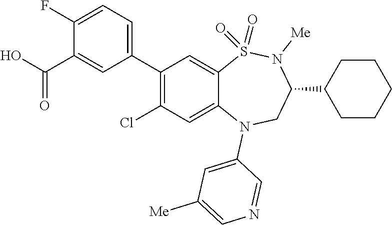 [SMILES: Cc1cncc(N2C[C@@H](C3CCCCC3)N(C)S(=O)(=O)c3cc(-c4ccc(F)c(C(=O)O)c4)c(Cl)cc32)c1] |
| 282 | 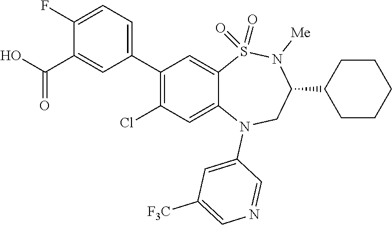 [SMILES: CN1[C@H](C2CCCCC2)CN(c2cncc(C(F)(F)F)c2)c2cc(Cl)c(-c3ccc(F)c(C(=O)O)c3)cc2S1(=O)=O] |
| 283 | 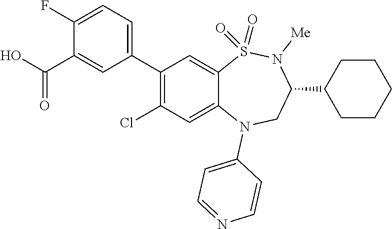 [SMILES: CN1[C@H](C2CCCCC2)CN(c2ccncc2)c2cc(Cl)c(-c3ccc(F)c(C(=O)O)c3)cc2S1(=O)=O] |
| 284 | 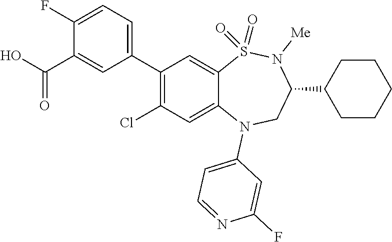 [SMILES: CN1[C@H](C2CCCCC2)CN(c2ccnc(F)c2)c2cc(Cl)c(-c3ccc(F)c(C(=O)O)c3)cc2S1(=O)=O] |
| 285 | 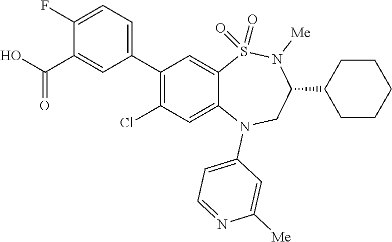 [SMILES: Cc1cc(N2C[C@@H](C3CCCCC3)N(C)S(=O)(=O)c3cc(-c4ccc(F)c(C(=O)O)c4)c(Cl)cc32)ccn1] |
| 286 | 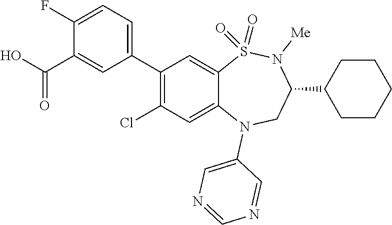 [SMILES: CN1[C@H](C2CCCCC2)CN(c2cncnc2)c2cc(Cl)c(-c3ccc(F)c(C(=O)O)c3)cc2S1(=O)=O] |
| 287 | 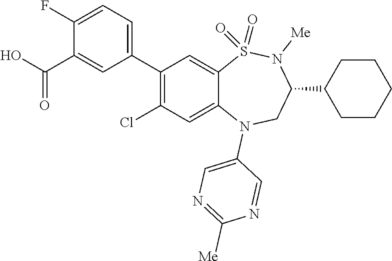 [SMILES: Cc1ncc(N2C[C@@H](C3CCCCC3)N(C)S(=O)(=O)c3cc(-c4ccc(F)c(C(=O)O)c4)c(Cl)cc32)cn1] |
| 288 | 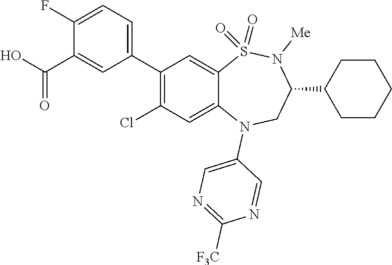 [SMILES: CN1[C@H](C2CCCCC2)CN(c2cnc(C(F)(F)F)nc2)c2cc(Cl)c(-c3ccc(F)c(C(=O)O)c3)cc2S1(=O)=O] |
| 289 | 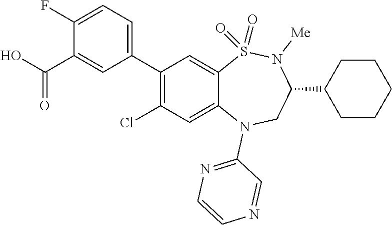 [SMILES: CN1[C@H](C2CCCCC2)CN(c2cnccn2)c2cc(Cl)c(-c3ccc(F)c(C(=O)O)c3)cc2S1(=O)=O] |
| 290 | 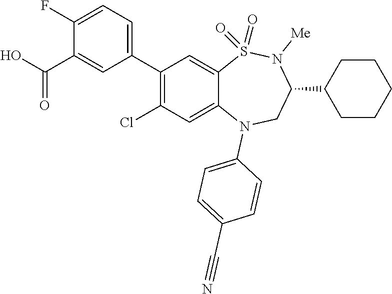 [SMILES: CN1[C@H](C2CCCCC2)CN(c2ccc(C#N)cc2)c2cc(Cl)c(-c3ccc(F)c(C(=O)O)c3)cc2S1(=O)=O] |
| 291 | 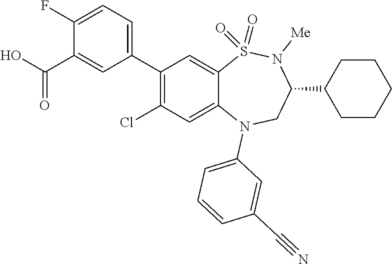 [SMILES: CN1[C@H](C2CCCCC2)CN(c2cccc(C#N)c2)c2cc(Cl)c(-c3ccc(F)c(C(=O)O)c3)cc2S1(=O)=O] |
| 292 | 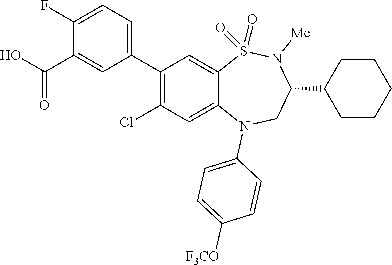 [SMILES: CN1[C@H](C2CCCCC2)CN(c2ccc(OC(F)(F)F)cc2)c2cc(Cl)c(-c3ccc(F)c(C(=O)O)c3)cc2S1(=O)=O] |
| 293 | 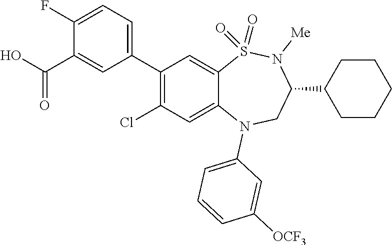 [SMILES: CN1[C@H](C2CCCCC2)CN(c2cccc(OC(F)(F)F)c2)c2cc(Cl)c(-c3ccc(F)c(C(=O)O)c3)cc2S1(=O)=O] |
| 294 | 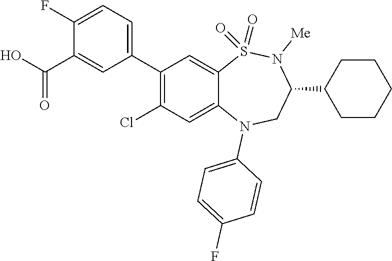 [SMILES: CN1[C@H](C2CCCCC2)CN(c2ccc(F)cc2)c2cc(Cl)c(-c3ccc(F)c(C(=O)O)c3)cc2S1(=O)=O] |
| 295 | 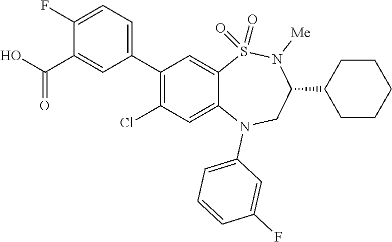 [SMILES: CN1[C@H](C2CCCCC2)CN(c2cccc(F)c2)c2cc(Cl)c(-c3ccc(F)c(C(=O)O)c3)cc2S1(=O)=O] |
| 296 | 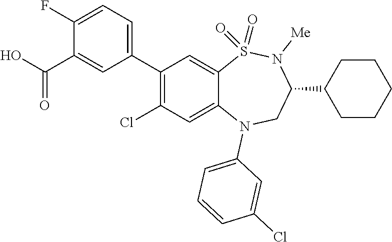 [SMILES: CN1[C@H](C2CCCCC2)CN(c2cccc(Cl)c2)c2cc(Cl)c(-c3ccc(F)c(C(=O)O)c3)cc2S1(=O)=O] |
| 297 | 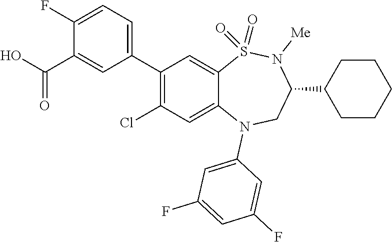 [SMILES: CN1[C@H](C2CCCCC2)CN(c2cc(F)cc(F)c2)c2cc(Cl)c(-c3ccc(F)c(C(=O)O)c3)cc2S1(=O)=O] |
| 298 | 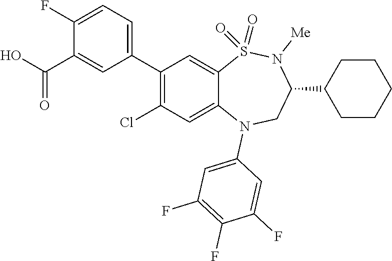 [SMILES: CN1[C@H](C2CCCCC2)CN(c2cc(F)c(F)c(F)c2)c2cc(Cl)c(-c3ccc(F)c(C(=O)O)c3)cc2S1(=O)=O] |
| 299 | 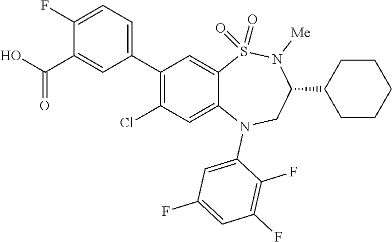 [SMILES: CN1[C@H](C2CCCCC2)CN(c2cc(F)cc(F)c2F)c2cc(Cl)c(-c3ccc(F)c(C(=O)O)c3)cc2S1(=O)=O] |
| 300 | 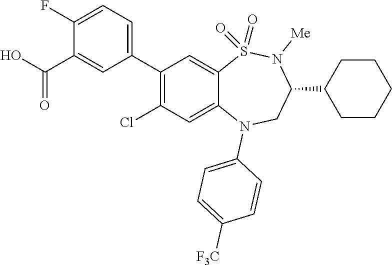 [SMILES: CN1[C@H](C2CCCCC2)CN(c2ccc(C(F)(F)F)cc2)c2cc(Cl)c(-c3ccc(F)c(C(=O)O)c3)cc2S1(=O)=O] |
| 301 | 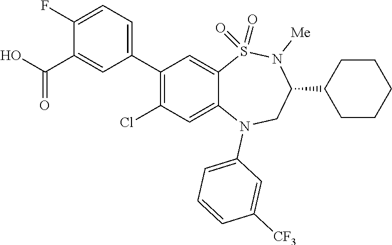 [SMILES: CN1[C@H](C2CCCCC2)CN(c2cccc(C(F)(F)F)c2)c2cc(Cl)c(-c3ccc(F)c(C(=O)O)c3)cc2S1(=O)=O] |
| 302 | 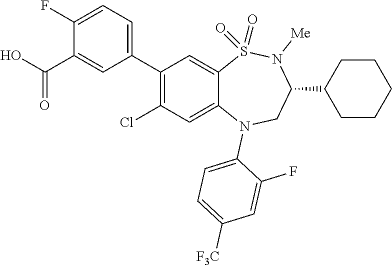 [SMILES: CN1[C@H](C2CCCCC2)CN(c2ccc(C(F)(F)F)cc2F)c2cc(Cl)c(-c3ccc(F)c(C(=O)O)c3)cc2S1(=O)=O] |
| 303 | 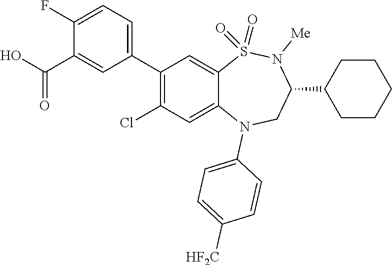 |
| 304 | 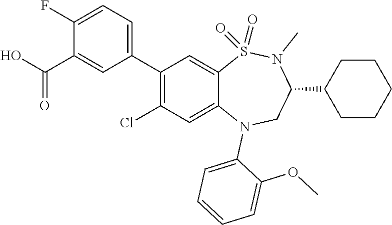 [SMILES: COc1ccccc1N1C[C@@H](C2CCCCC2)N(C)S(=O)(=O)c2cc(-c3ccc(F)c(C(=O)O)c3)c(Cl)cc21] |
| 305 | 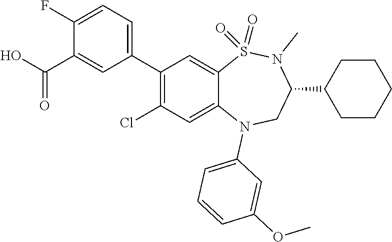 [SMILES: COc1cccc(N2C[C@@H](C3CCCCC3)N(C)S(=O)(=O)c3cc(-c4ccc(F)c(C(=O)O)c4)c(Cl)cc32)c1] |
| 306 | 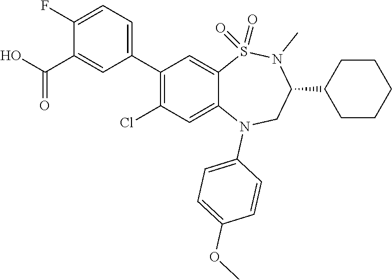 [SMILES: COc1ccc(N2C[C@@H](C3CCCCC3)N(C)S(=O)(=O)c3cc(-c4ccc(F)c(C(=O)O)c4)c(Cl)cc32)cc1] |
| 307 | 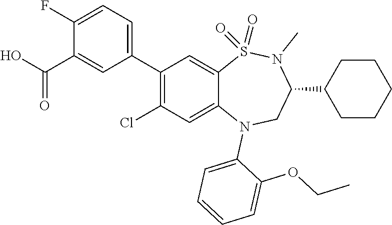 [SMILES: CCOc1ccccc1N1C[C@@H](C2CCCCC2)N(C)S(=O)(=O)c2cc(-c3ccc(F)c(C(=O)O)c3)c(Cl)cc21] |
| 308 | 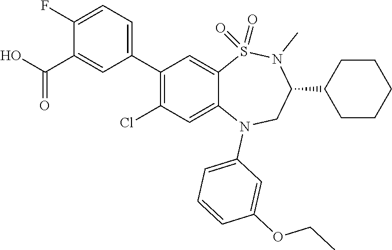 [SMILES: CCOc1cccc(N2C[C@@H](C3CCCCC3)N(C)S(=O)(=O)c3cc(-c4ccc(F)c(C(=O)O)c4)c(Cl)cc32)c1] |
| 309 | 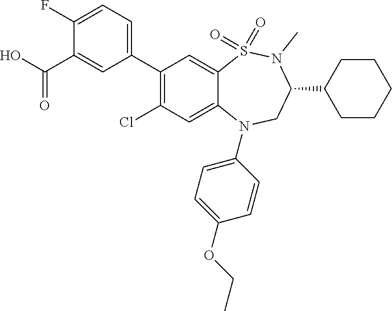 [SMILES: CCOc1ccc(N2C[C@@H](C3CCCCC3)N(C)S(=O)(=O)c3cc(-c4ccc(F)c(C(=O)O)c4)c(Cl)cc32)cc1] |
| 310 | 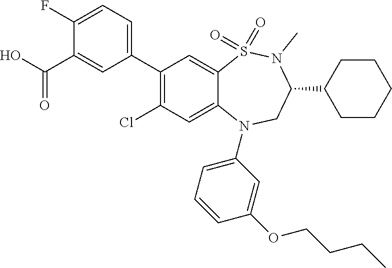 [SMILES: CCCCOc1cccc(N2C[C@@H](C3CCCCC3)N(C)S(=O)(=O)c3cc(-c4ccc(F)c(C(=O)O)c4)c(Cl)cc32)c1] |
| 311 | 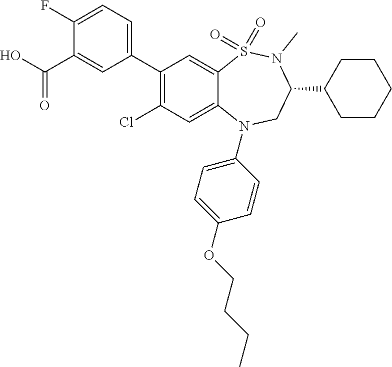 [SMILES: CCCCOc1ccc(N2C[C@@H](C3CCCCC3)N(C)S(=O)(=O)c3cc(-c4ccc(F)c(C(=O)O)c4)c(Cl)cc32)cc1] |
| 312 | 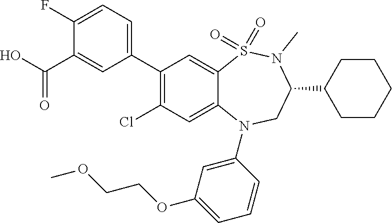 [SMILES: COCCOc1cccc(N2C[C@@H](C3CCCCC3)N(C)S(=O)(=O)c3cc(-c4ccc(F)c(C(=O)O)c4)c(Cl)cc32)c1] |
| 313 | 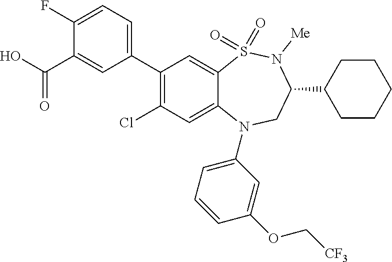 [SMILES: CN1[C@H](C2CCCCC2)CN(c2cccc(OCC(F)(F)F)c2)c2cc(Cl)c(-c3ccc(F)c(C(=O)O)c3)cc2S1(=O)=O] |
| 314 | 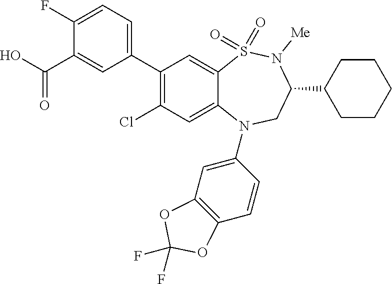 [SMILES: CN1[C@H](C2CCCCC2)CN(c2ccc3c(c2)OC(F)(F)O3)c2cc(Cl)c(-c3ccc(F)c(C(=O)O)c3)cc2S1(=O)=O] |
| 315 | 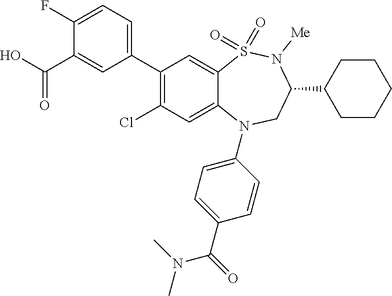 [SMILES: CN(C)C(=O)c1ccc(N2C[C@@H](C3CCCCC3)N(C)S(=O)(=O)c3cc(-c4ccc(F)c(C(=O)O)c4)c(Cl)cc32)cc1] |
| 316 | 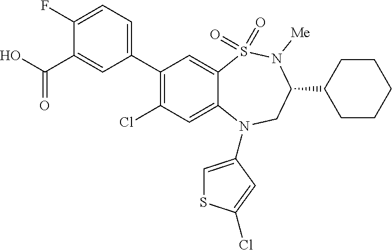 [SMILES: CN1[C@H](C2CCCCC2)CN(c2csc(Cl)c2)c2cc(Cl)c(-c3ccc(F)c(C(=O)O)c3)cc2S1(=O)=O] |
| 317 | 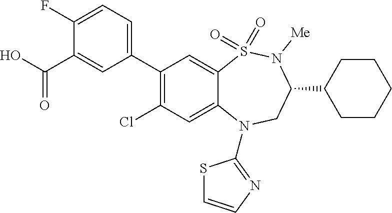 [SMILES: CN1[C@H](C2CCCCC2)CN(c2nccs2)c2cc(Cl)c(-c3ccc(F)c(C(=O)O)c3)cc2S1(=O)=O] |
| 318 | 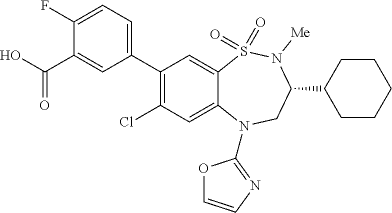 [SMILES: CN1[C@H](C2CCCCC2)CN(c2ncco2)c2cc(Cl)c(-c3ccc(F)c(C(=O)O)c3)cc2S1(=O)=O] |
| 319 | 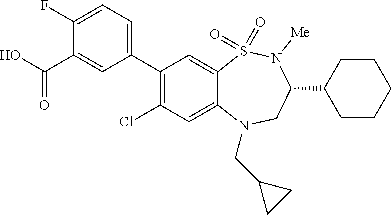 [SMILES: CN1[C@H](C2CCCCC2)CN(CC2CC2)c2cc(Cl)c(-c3ccc(F)c(C(=O)O)c3)cc2S1(=O)=O] |
| 320 | 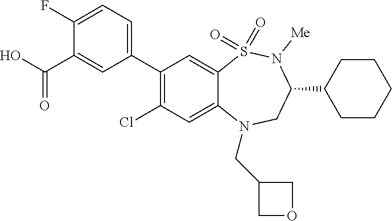 [SMILES: CN1[C@H](C2CCCCC2)CN(CC2COC2)c2cc(Cl)c(-c3ccc(F)c(C(=O)O)c3)cc2S1(=O)=O] |
| 321 | 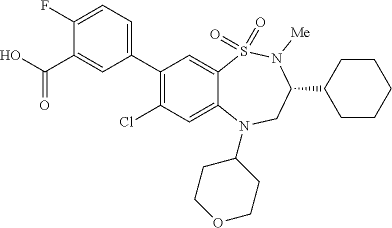 [SMILES: CN1[C@H](C2CCCCC2)CN(C2CCOCC2)c2cc(Cl)c(-c3ccc(F)c(C(=O)O)c3)cc2S1(=O)=O] |
| 322 | 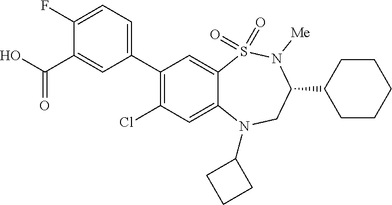 [SMILES: CN1[C@H](C2CCCCC2)CN(C2CCC2)c2cc(Cl)c(-c3ccc(F)c(C(=O)O)c3)cc2S1(=O)=O] |
| 323 | 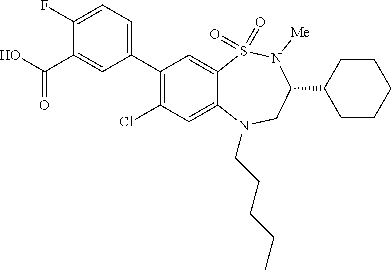 [SMILES: CCCCCN1C[C@@H](C2CCCCC2)N(C)S(=O)(=O)c2cc(-c3ccc(F)c(C(=O)O)c3)c(Cl)cc21] |
| 324 | 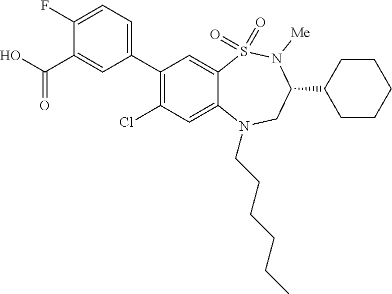 [SMILES: CCCCCCN1C[C@@H](C2CCCCC2)N(C)S(=O)(=O)c2cc(-c3ccc(F)c(C(=O)O)c3)c(Cl)cc21] |
| 325 | 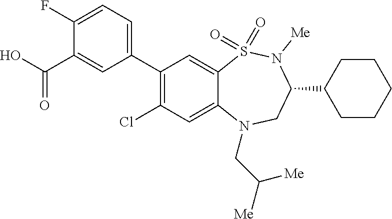 [SMILES: CC(C)CN1C[C@@H](C2CCCCC2)N(C)S(=O)(=O)c2cc(-c3ccc(F)c(C(=O)O)c3)c(Cl)cc21] |
| 326 | 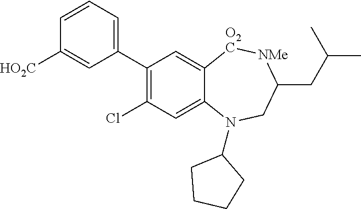 |
| 327 | 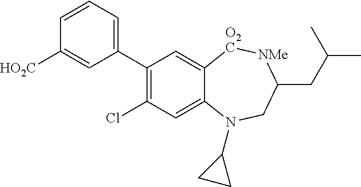 |
| 328 | 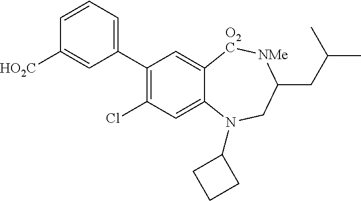 |
| 329 | 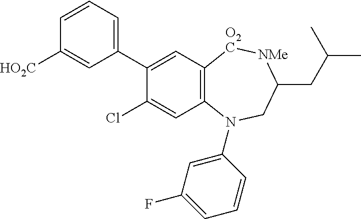 |
| 330 | 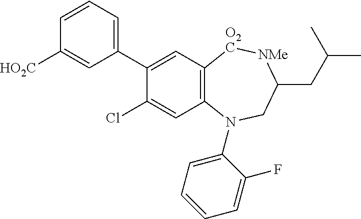 |
| 331 | 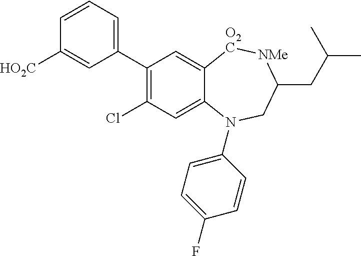 |
| 332 | 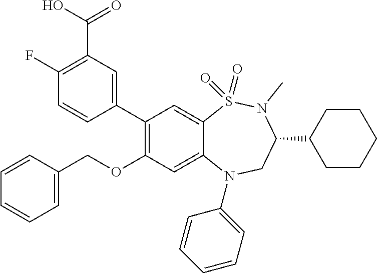 [SMILES: CN1[C@H](C2CCCCC2)CN(c2ccccc2)c2cc(OCc3ccccc3)c(-c3ccc(F)c(C(=O)O)c3)cc2S1(=O)=O] |
| 333 | 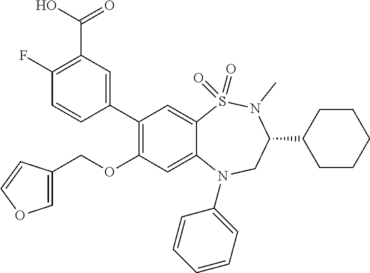 [SMILES: CN1[C@H](C2CCCCC2)CN(c2ccccc2)c2cc(OCc3ccoc3)c(-c3ccc(F)c(C(=O)O)c3)cc2S1(=O)=O] |
| 334 | 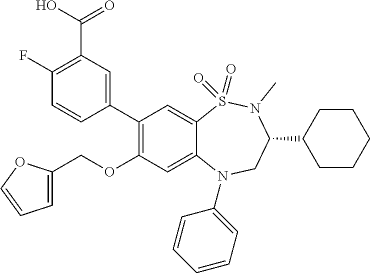 [SMILES: CN1[C@H](C2CCCCC2)CN(c2ccccc2)c2cc(OCc3ccco3)c(-c3ccc(F)c(C(=O)O)c3)cc2S1(=O)=O] |
| 335 | 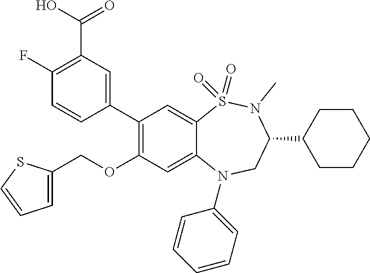 [SMILES: CN1[C@H](C2CCCCC2)CN(c2ccccc2)c2cc(OCc3cccs3)c(-c3ccc(F)c(C(=O)O)c3)cc2S1(=O)=O] |
| 336 | 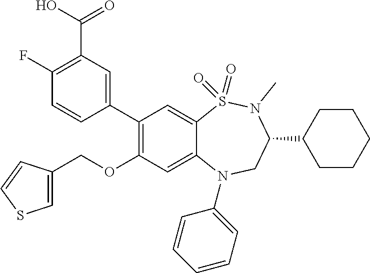 [SMILES: CN1[C@H](C2CCCCC2)CN(c2ccccc2)c2cc(OCc3ccsc3)c(-c3ccc(F)c(C(=O)O)c3)cc2S1(=O)=O] |
| 337 | 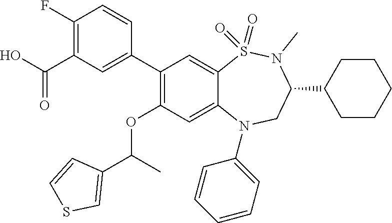 [SMILES: CC(Oc1cc2c(cc1-c1ccc(F)c(C(=O)O)c1)S(=O)(=O)N(C)[C@H](C1CCCCC1)CN2c1ccccc1)c1ccsc1] |
| 338 | 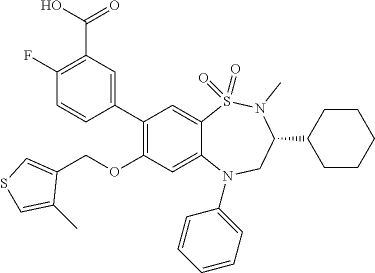 [SMILES: Cc1cscc1COc1cc2c(cc1-c1ccc(F)c(C(=O)O)c1)S(=O)(=O)N(C)[C@H](C1CCCCC1)CN2c1ccccc1] |
| 339 | 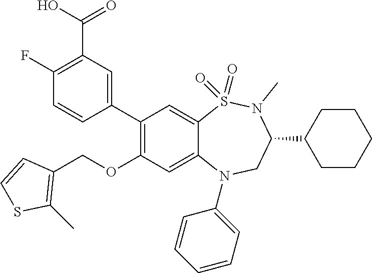 [SMILES: Cc1sccc1COc1cc2c(cc1-c1ccc(F)c(C(=O)O)c1)S(=O)(=O)N(C)[C@H](C1CCCCC1)CN2c1ccccc1] |
| 340 | 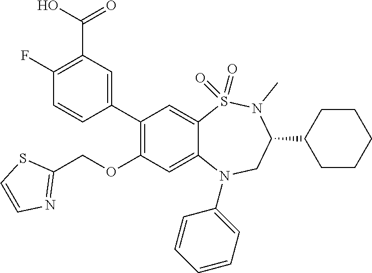 [SMILES: CN1[C@H](C2CCCCC2)CN(c2ccccc2)c2cc(OCc3nccs3)c(-c3ccc(F)c(C(=O)O)c3)cc2S1(=O)=O] |
| 341 | 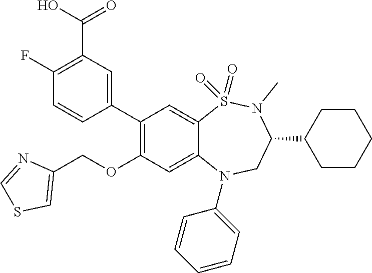 [SMILES: CN1[C@H](C2CCCCC2)CN(c2ccccc2)c2cc(OCc3cscn3)c(-c3ccc(F)c(C(=O)O)c3)cc2S1(=O)=O] |
| 342 | 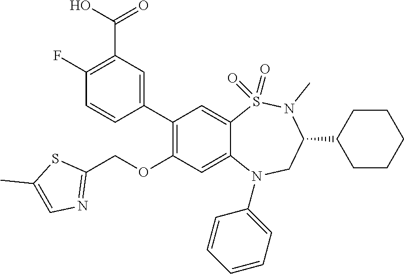 [SMILES: Cc1cnc(COc2cc3c(cc2-c2ccc(F)c(C(=O)O)c2)S(=O)(=O)N(C)[C@H](C2CCCCC2)CN3c2ccccc2)s1] |
| 343 | 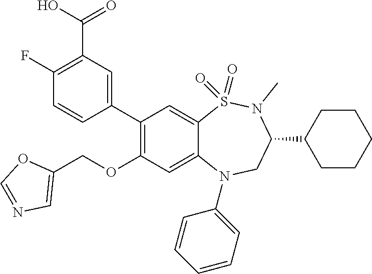 [SMILES: CN1[C@H](C2CCCCC2)CN(c2ccccc2)c2cc(OCc3cnco3)c(-c3ccc(F)c(C(=O)O)c3)cc2S1(=O)=O] |
| 344 | 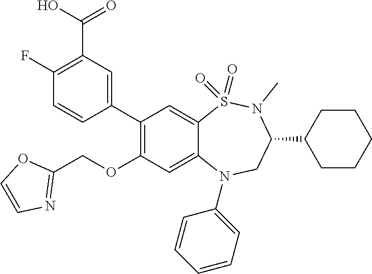 [SMILES: CN1[C@H](C2CCCCC2)CN(c2ccccc2)c2cc(OCc3ncco3)c(-c3ccc(F)c(C(=O)O)c3)cc2S1(=O)=O] |
| 345 | 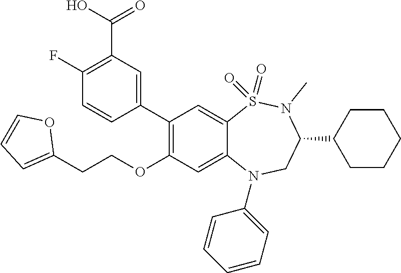 [SMILES: CN1[C@H](C2CCCCC2)CN(c2ccccc2)c2cc(OCCc3ccco3)c(-c3ccc(F)c(C(=O)O)c3)cc2S1(=O)=O] |
| 346 | 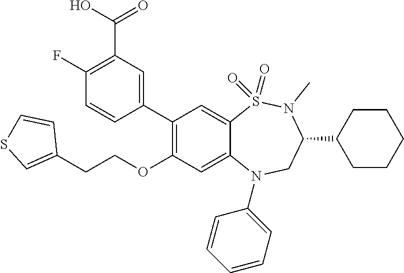 [SMILES: CN1[C@H](C2CCCCC2)CN(c2ccccc2)c2cc(OCCc3ccsc3)c(-c3ccc(F)c(C(=O)O)c3)cc2S1(=O)=O] |
| 347 | 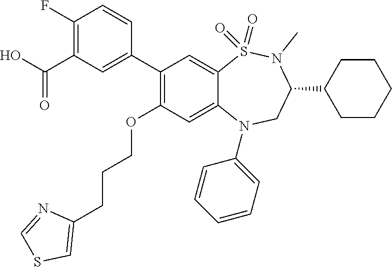 [SMILES: CN1[C@H](C2CCCCC2)CN(c2ccccc2)c2cc(OCCCc3cscn3)c(-c3ccc(F)c(C(=O)O)c3)cc2S1(=O)=O] |
| 348 | 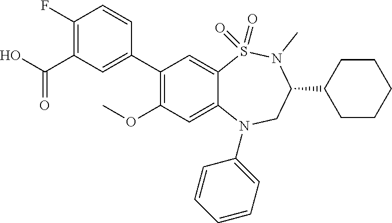 [SMILES: COc1cc2c(cc1-c1ccc(F)c(C(=O)O)c1)S(=O)(=O)N(C)[C@H](C1CCCCC1)CN2c1ccccc1] |
| 349 | 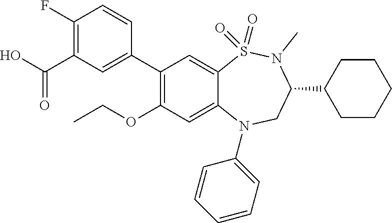 [SMILES: CCOc1cc2c(cc1-c1ccc(F)c(C(=O)O)c1)S(=O)(=O)N(C)[C@H](C1CCCCC1)CN2c1ccccc1] |
| 350 | 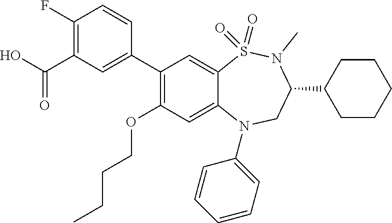 [SMILES: CCCCOc1cc2c(cc1-c1ccc(F)c(C(=O)O)c1)S(=O)(=O)N(C)[C@H](C1CCCCC1)CN2c1ccccc1] |
| 351 | 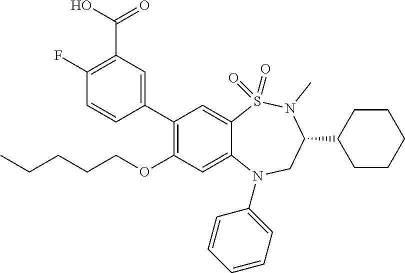 [SMILES: CCCCCOc1cc2c(cc1-c1ccc(F)c(C(=O)O)c1)S(=O)(=O)N(C)[C@H](C1CCCCC1)CN2c1ccccc1] |
| 352 | 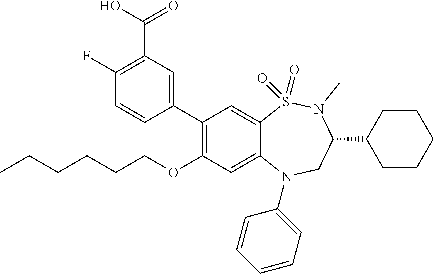 [SMILES: CCCCCCOc1cc2c(cc1-c1ccc(F)c(C(=O)O)c1)S(=O)(=O)N(C)[C@H](C1CCCCC1)CN2c1ccccc1] |
| 353 | 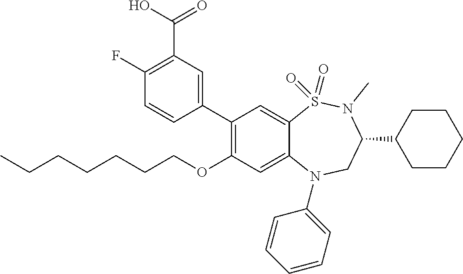 [SMILES: CCCCCCCOc1cc2c(cc1-c1ccc(F)c(C(=O)O)c1)S(=O)(=O)N(C)[C@H](C1CCCCC1)CN2c1ccccc1] |
| 354 | 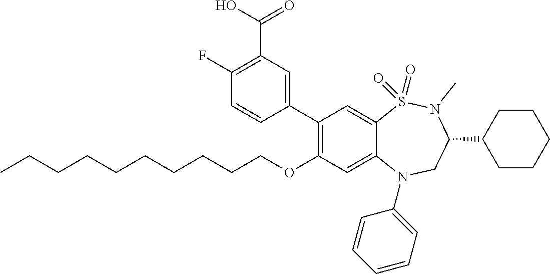 [SMILES: CCCCCCCCCCOc1cc2c(cc1-c1ccc(F)c(C(=O)O)c1)S(=O)(=O)N(C)[C@H](C1CCCCC1)CN2c1ccccc1] |
| 355 | 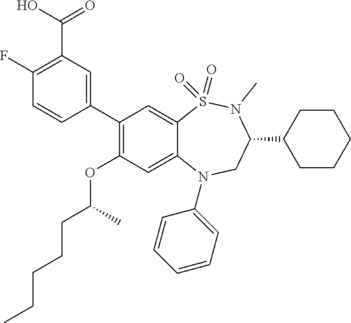 [SMILES: CCCCC[C@@H](C)Oc1cc2c(cc1-c1ccc(F)c(C(=O)O)c1)S(=O)(=O)N(C)[C@H](C1CCCCC1)CN2c1ccccc1] |
| 356 | 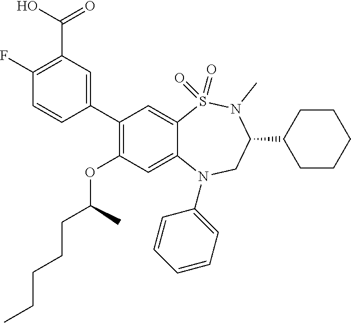 [SMILES: CCCCC[C@H](C)Oc1cc2c(cc1-c1ccc(F)c(C(=O)O)c1)S(=O)(=O)N(C)[C@H](C1CCCCC1)CN2c1ccccc1] |
| 357 | 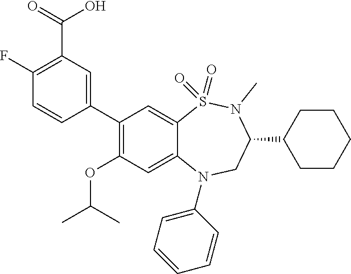 [SMILES: CC(C)Oc1cc2c(cc1-c1ccc(F)c(C(=O)O)c1)S(=O)(=O)N(C)[C@H](C1CCCCC1)CN2c1ccccc1] |
| 358 | 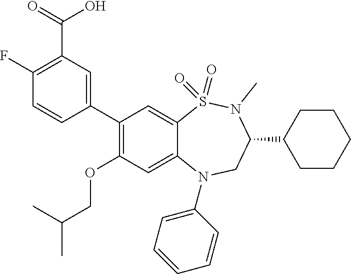 [SMILES: CC(C)COc1cc2c(cc1-c1ccc(F)c(C(=O)O)c1)S(=O)(=O)N(C)[C@H](C1CCCCC1)CN2c1ccccc1] |
| 359 | 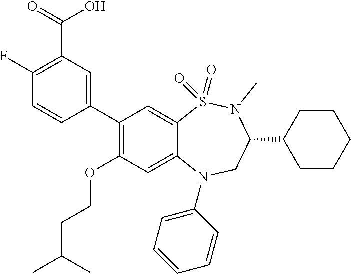 [SMILES: CC(C)CCOc1cc2c(cc1-c1ccc(F)c(C(=O)O)c1)S(=O)(=O)N(C)[C@H](C1CCCCC1)CN2c1ccccc1] |
| 360 | 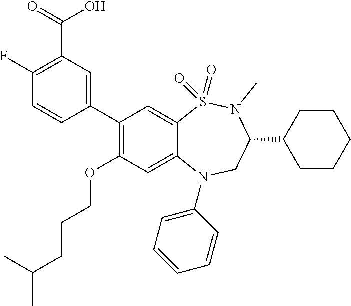 [SMILES: CC(C)CCCOc1cc2c(cc1-c1ccc(F)c(C(=O)O)c1)S(=O)(=O)N(C)[C@H](C1CCCCC1)CN2c1ccccc1] |
| 361 | 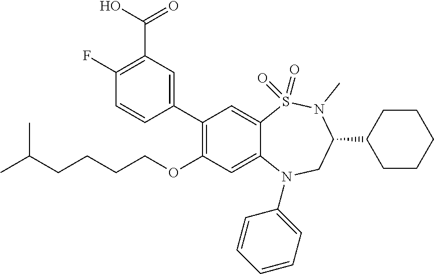 [SMILES: CC(C)CCCCOc1cc2c(cc1-c1ccc(F)c(C(=O)O)c1)S(=O)(=O)N(C)[C@H](C1CCCCC1)CN2c1ccccc1] |
| 362 | 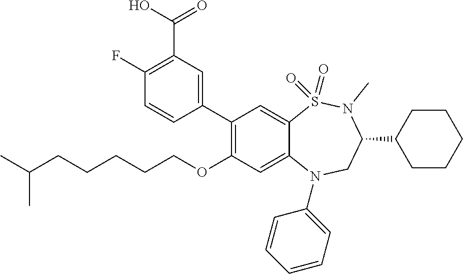 [SMILES: CC(C)CCCCCOc1cc2c(cc1-c1ccc(F)c(C(=O)O)c1)S(=O)(=O)N(C)[C@H](C1CCCCC1)CN2c1ccccc1] |
| 363 | 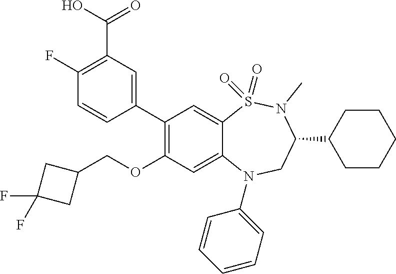 [SMILES: CN1[C@H](C2CCCCC2)CN(c2ccccc2)c2cc(OCC3CC(F)(F)C3)c(-c3ccc(F)c(C(=O)O)c3)cc2S1(=O)=O] |
| 364 | 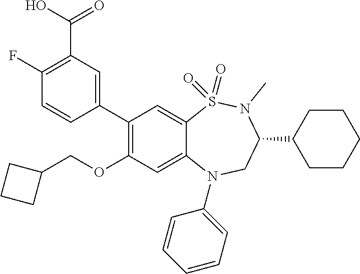 [SMILES: CN1[C@H](C2CCCCC2)CN(c2ccccc2)c2cc(OCC3CCC3)c(-c3ccc(F)c(C(=O)O)c3)cc2S1(=O)=O] |
| 365 | 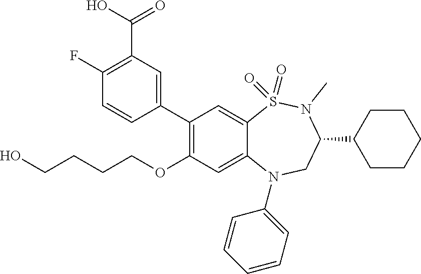 [SMILES: CN1[C@H](C2CCCCC2)CN(c2ccccc2)c2cc(OCCCCO)c(-c3ccc(F)c(C(=O)O)c3)cc2S1(=O)=O] |
| 366 | 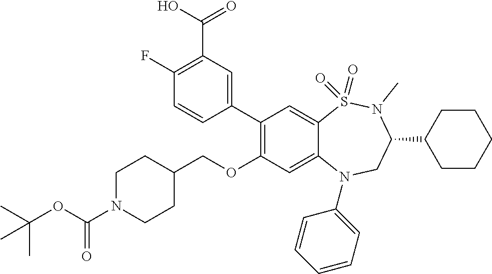 [SMILES: CN1[C@H](C2CCCCC2)CN(c2ccccc2)c2cc(OCC3CCN(C(=O)OC(C)(C)C)CC3)c(-c3ccc(F)c(C(=O)O)c3)cc2S1(=O)=O] |
| 367 | 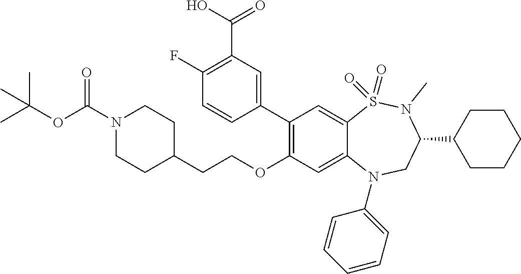 [SMILES: CN1[C@H](C2CCCCC2)CN(c2ccccc2)c2cc(OCCC3CCN(C(=O)OC(C)(C)C)CC3)c(-c3ccc(F)c(C(=O)O)c3)cc2S1(=O)=O] |
| 368 | 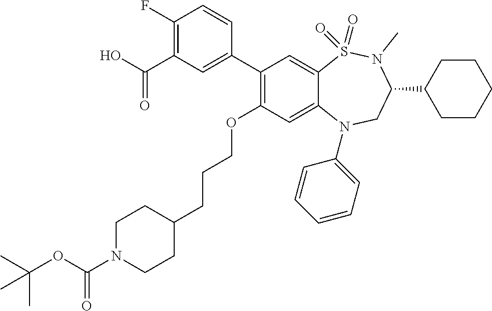 [SMILES: CN1[C@H](C2CCCCC2)CN(c2ccccc2)c2cc(OCCCC3CCN(C(=O)OC(C)(C)C)CC3)c(-c3ccc(F)c(C(=O)O)c3)cc2S1(=O)=O] |
| 369 | 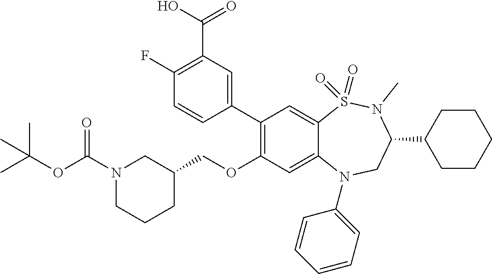 [SMILES: CN1[C@H](C2CCCCC2)CN(c2ccccc2)c2cc(OC[C@@H]3CCCN(C(=O)OC(C)(C)C)C3)c(-c3ccc(F)c(C(=O)O)c3)cc2S1(=O)=O] |
| 370 | 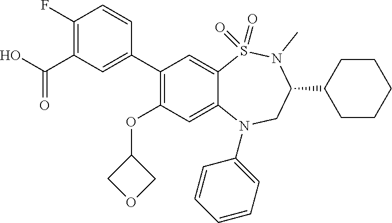 [SMILES: CN1[C@H](C2CCCCC2)CN(c2ccccc2)c2cc(OC3COC3)c(-c3ccc(F)c(C(=O)O)c3)cc2S1(=O)=O] |
| 371 | 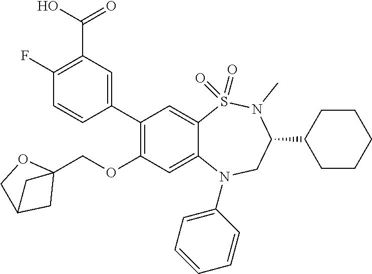 [SMILES: CN1[C@H](C2CCCCC2)CN(c2ccccc2)c2cc(OCC34CC(CO3)C4)c(-c3ccc(F)c(C(=O)O)c3)cc2S1(=O)=O] |
| 372 | 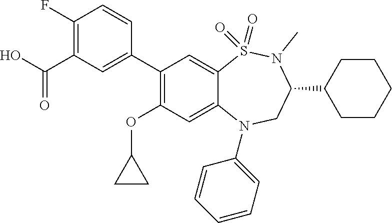 [SMILES: CN1[C@H](C2CCCCC2)CN(c2ccccc2)c2cc(OC3CC3)c(-c3ccc(F)c(C(=O)O)c3)cc2S1(=O)=O] |
| 373 | 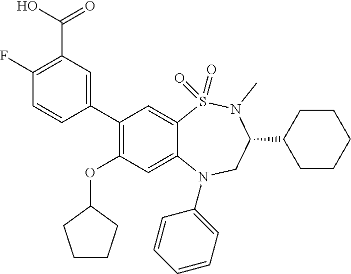 [SMILES: CN1[C@H](C2CCCCC2)CN(c2ccccc2)c2cc(OC3CCCC3)c(-c3ccc(F)c(C(=O)O)c3)cc2S1(=O)=O] |
| 374 | 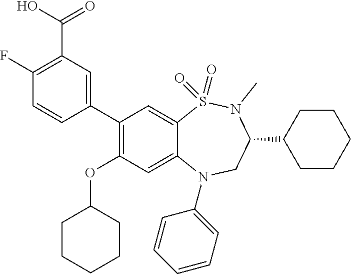 [SMILES: CN1[C@H](C2CCCCC2)CN(c2ccccc2)c2cc(OC3CCCCC3)c(-c3ccc(F)c(C(=O)O)c3)cc2S1(=O)=O] |
| 375 | 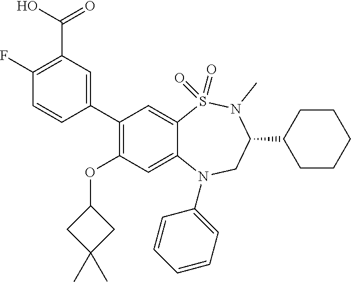 [SMILES: CN1[C@H](C2CCCCC2)CN(c2ccccc2)c2cc(OC3CC(C)(C)C3)c(-c3ccc(F)c(C(=O)O)c3)cc2S1(=O)=O] |
| 376 | 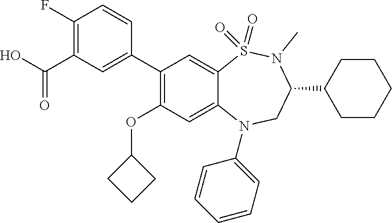 [SMILES: CN1[C@H](C2CCCCC2)CN(c2ccccc2)c2cc(OC3CCC3)c(-c3ccc(F)c(C(=O)O)c3)cc2S1(=O)=O] |
| 377 | 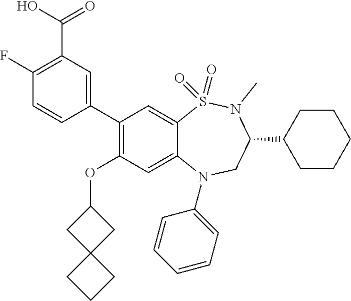 [SMILES: CN1[C@H](C2CCCCC2)CN(c2ccccc2)c2cc(OC3CC4(CCC4)C3)c(-c3ccc(F)c(C(=O)O)c3)cc2S1(=O)=O] |
| 378 | 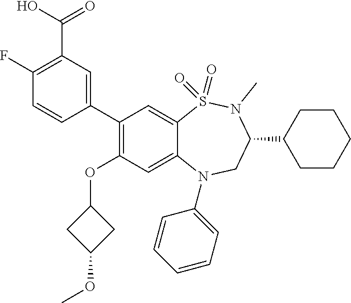 |
| 379 | 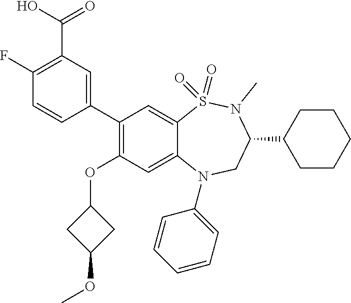 |
| 380 | 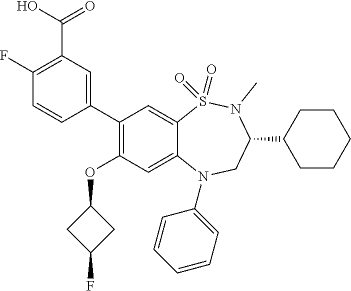 [SMILES: CN1[C@H](C2CCCCC2)CN(c2ccccc2)c2cc(O[C@H]3C[C@@H](F)C3)c(-c3ccc(F)c(C(=O)O)c3)cc2S1(=O)=O] |
| 381 | 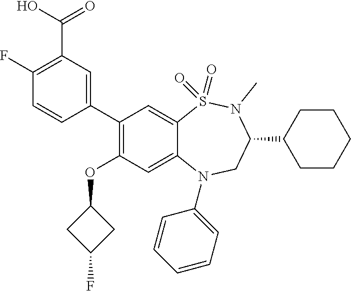 [SMILES: CN1[C@H](C2CCCCC2)CN(c2ccccc2)c2cc(O[C@H]3C[C@H](F)C3)c(-c3ccc(F)c(C(=O)O)c3)cc2S1(=O)=O] |
| 382 | 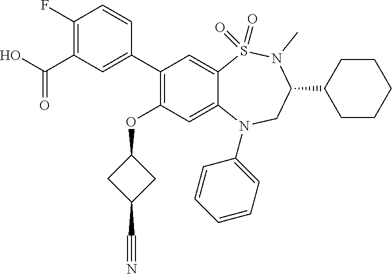 [SMILES: CN1[C@H](C2CCCCC2)CN(c2ccccc2)c2cc(O[C@H]3C[C@@H](C#N)C3)c(-c3ccc(F)c(C(=O)O)c3)cc2S1(=O)=O] |
| 383 | 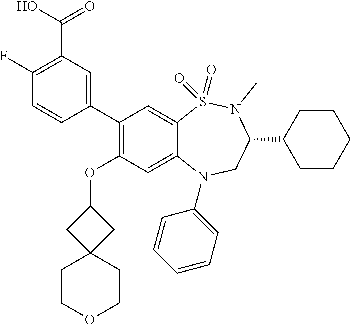 [SMILES: CN1[C@H](C2CCCCC2)CN(c2ccccc2)c2cc(OC3CC4(CCOCC4)C3)c(-c3ccc(F)c(C(=O)O)c3)cc2S1(=O)=O] |
| 384 | 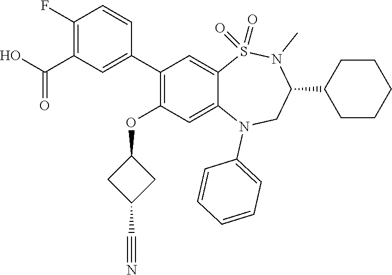 [SMILES: CN1[C@H](C2CCCCC2)CN(c2ccccc2)c2cc(O[C@H]3C[C@H](C#N)C3)c(-c3ccc(F)c(C(=O)O)c3)cc2S1(=O)=O] |
| 385 | 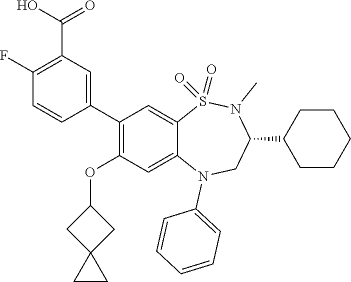 [SMILES: CN1[C@H](C2CCCCC2)CN(c2ccccc2)c2cc(OC3CC4(CC4)C3)c(-c3ccc(F)c(C(=O)O)c3)cc2S1(=O)=O] |
| 386 | 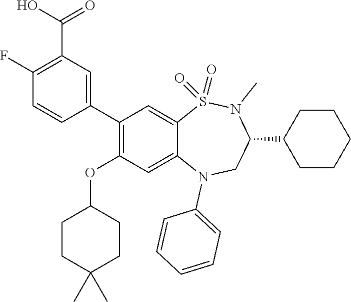 [SMILES: CN1[C@H](C2CCCCC2)CN(c2ccccc2)c2cc(OC3CCC(C)(C)CC3)c(-c3ccc(F)c(C(=O)O)c3)cc2S1(=O)=O] |
| 387 | 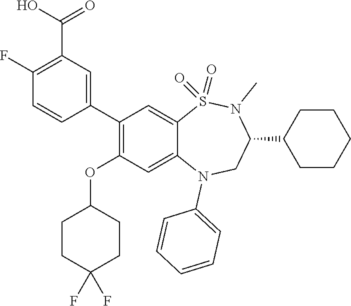 [SMILES: CN1[C@H](C2CCCCC2)CN(c2ccccc2)c2cc(OC3CCC(F)(F)CC3)c(-c3ccc(F)c(C(=O)O)c3)cc2S1(=O)=O] |
| 388 | 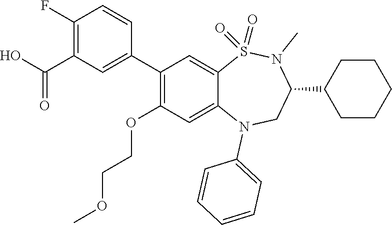 [SMILES: COCCOc1cc2c(cc1-c1ccc(F)c(C(=O)O)c1)S(=O)(=O)N(C)[C@H](C1CCCCC1)CN2c1ccccc1] |
| 389 | 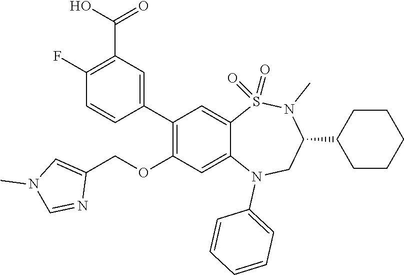 [SMILES: CN1[C@H](C2CCCCC2)CN(c2ccccc2)c2cc(OCc3cn(C)cn3)c(-c3ccc(F)c(C(=O)O)c3)cc2S1(=O)=O] |
| 390 | 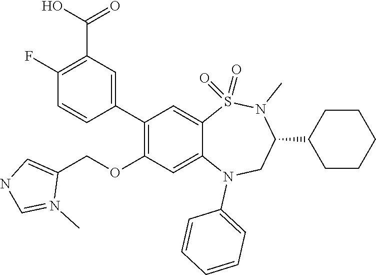 |
| 391 | 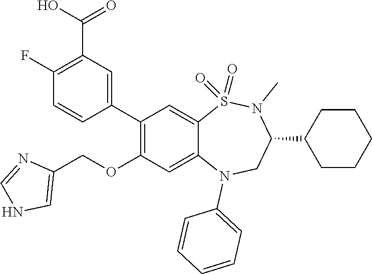 [SMILES: CN1[C@H](C2CCCCC2)CN(c2ccccc2)c2cc(OCc3c[nH]cn3)c(-c3ccc(F)c(C(=O)O)c3)cc2S1(=O)=O] |
| 392 | 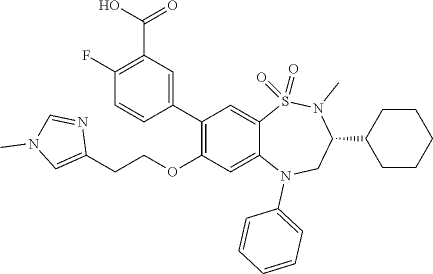 [SMILES: CN1[C@H](C2CCCCC2)CN(c2ccccc2)c2cc(OCCc3cn(C)cn3)c(-c3ccc(F)c(C(=O)O)c3)cc2S1(=O)=O] |
| 393 | 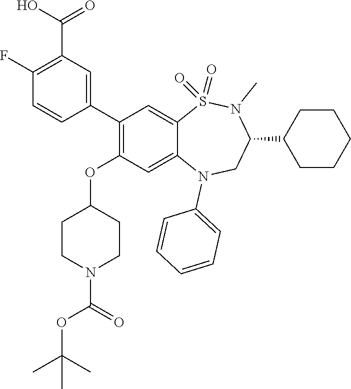 [SMILES: CN1[C@H](C2CCCCC2)CN(c2ccccc2)c2cc(OC3CCN(C(=O)OC(C)(C)C)CC3)c(-c3ccc(F)c(C(=O)O)c3)cc2S1(=O)=O] |
| 394 | 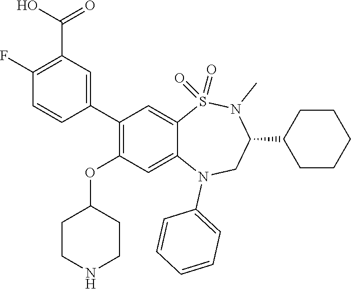 [SMILES: CN1[C@H](C2CCCCC2)CN(c2ccccc2)c2cc(OC3CCNCC3)c(-c3ccc(F)c(C(=O)O)c3)cc2S1(=O)=O] |
| 395 | 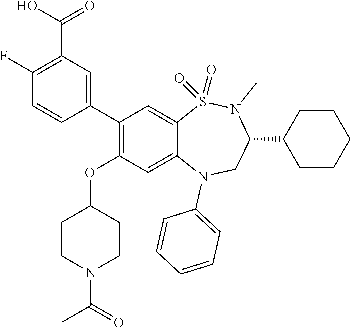 [SMILES: CC(=O)N1CCC(Oc2cc3c(cc2-c2ccc(F)c(C(=O)O)c2)S(=O)(=O)N(C)[C@H](C2CCCCC2)CN3c2ccccc2)CC1] |
| 396 | 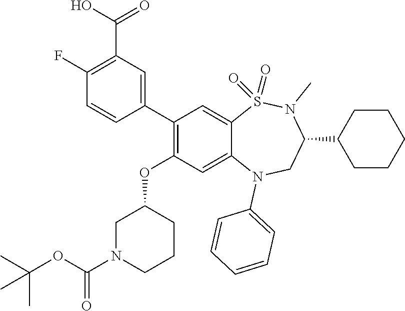 [SMILES: CN1[C@H](C2CCCCC2)CN(c2ccccc2)c2cc(O[C@@H]3CCCN(C(=O)OC(C)(C)C)C3)c(-c3ccc(F)c(C(=O)O)c3)cc2S1(=O)=O] |
| 397 | 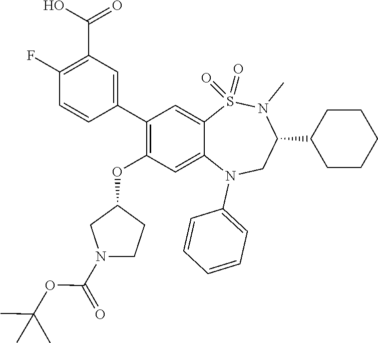 [SMILES: CN1[C@H](C2CCCCC2)CN(c2ccccc2)c2cc(O[C@@H]3CCN(C(=O)OC(C)(C)C)C3)c(-c3ccc(F)c(C(=O)O)c3)cc2S1(=O)=O] |
| 398 | 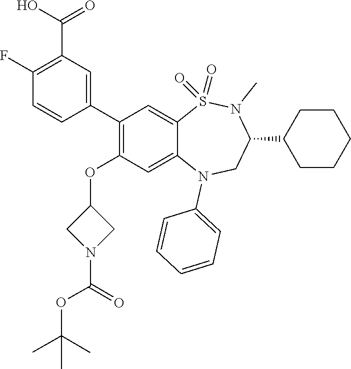 [SMILES: CN1[C@H](C2CCCCC2)CN(c2ccccc2)c2cc(OC3CN(C(=O)OC(C)(C)C)C3)c(-c3ccc(F)c(C(=O)O)c3)cc2S1(=O)=O] |
| 399 | 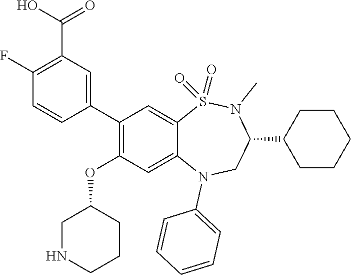 [SMILES: CN1[C@H](C2CCCCC2)CN(c2ccccc2)c2cc(O[C@@H]3CCCNC3)c(-c3ccc(F)c(C(=O)O)c3)cc2S1(=O)=O] |
| 400 | 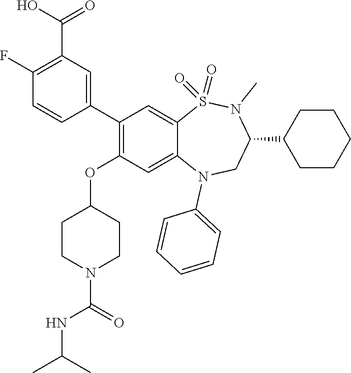 [SMILES: CC(C)NC(=O)N1CCC(Oc2cc3c(cc2-c2ccc(F)c(C(=O)O)c2)S(=O)(=O)N(C)[C@H](C2CCCCC2)CN3c2ccccc2)CC1] |
| 401 | 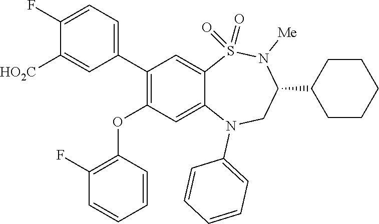 [SMILES: CN1[C@H](C2CCCCC2)CN(c2ccccc2)c2cc(Oc3ccccc3F)c(-c3ccc(F)c(C(=O)O)c3)cc2S1(=O)=O] |
| 402 | 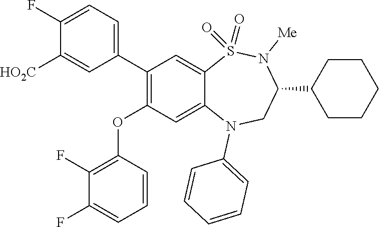 [SMILES: CN1[C@H](C2CCCCC2)CN(c2ccccc2)c2cc(Oc3cccc(F)c3F)c(-c3ccc(F)c(C(=O)O)c3)cc2S1(=O)=O] |
| 403 | 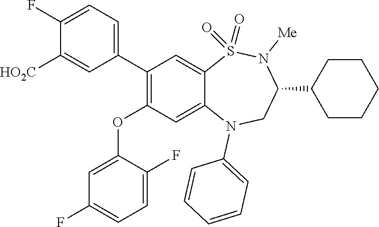 [SMILES: CN1[C@H](C2CCCCC2)CN(c2ccccc2)c2cc(Oc3cc(F)ccc3F)c(-c3ccc(F)c(C(=O)O)c3)cc2S1(=O)=O] |
| 404 | 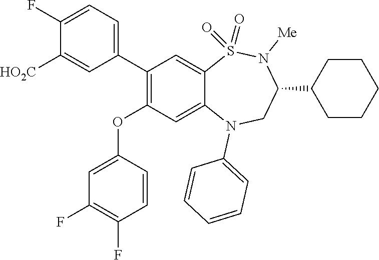 [SMILES: CN1[C@H](C2CCCCC2)CN(c2ccccc2)c2cc(Oc3ccc(F)c(F)c3)c(-c3ccc(F)c(C(=O)O)c3)cc2S1(=O)=O] |
| 405 | 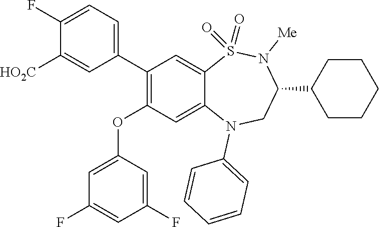 [SMILES: CN1[C@H](C2CCCCC2)CN(c2ccccc2)c2cc(Oc3cc(F)cc(F)c3)c(-c3ccc(F)c(C(=O)O)c3)cc2S1(=O)=O] |
| 406 | 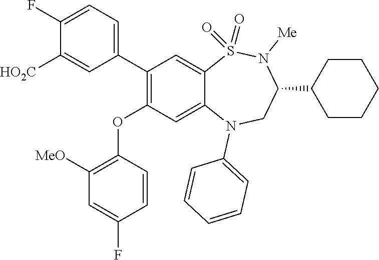 [SMILES: COc1cc(F)ccc1Oc1cc2c(cc1-c1ccc(F)c(C(=O)O)c1)S(=O)(=O)N(C)[C@H](C1CCCCC1)CN2c1ccccc1] |
| 407 | 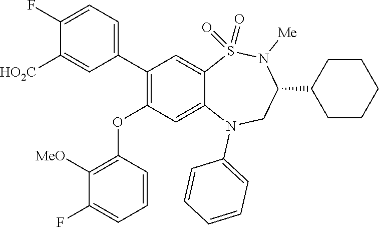 [SMILES: COc1c(F)cccc1Oc1cc2c(cc1-c1ccc(F)c(C(=O)O)c1)S(=O)(=O)N(C)[C@H](C1CCCCC1)CN2c1ccccc1] |
| 408 | 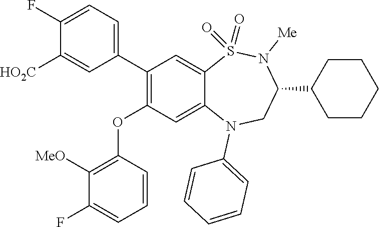 [SMILES: COc1c(F)cccc1Oc1cc2c(cc1-c1ccc(F)c(C(=O)O)c1)S(=O)(=O)N(C)[C@H](C1CCCCC1)CN2c1ccccc1] |
| 409 | 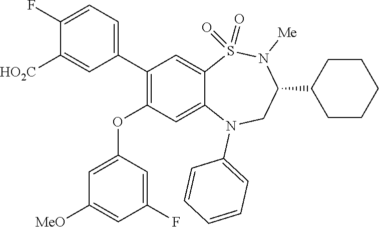 [SMILES: COc1cc(F)cc(Oc2cc3c(cc2-c2ccc(F)c(C(=O)O)c2)S(=O)(=O)N(C)[C@H](C2CCCCC2)CN3c2ccccc2)c1] |
| 410 | 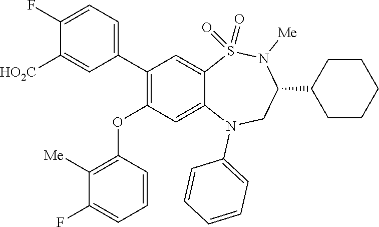 [SMILES: Cc1c(F)cccc1Oc1cc2c(cc1-c1ccc(F)c(C(=O)O)c1)S(=O)(=O)N(C)[C@H](C1CCCCC1)CN2c1ccccc1] |
| 411 | 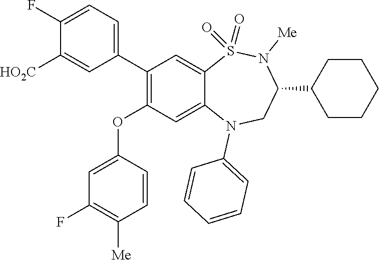 [SMILES: Cc1ccc(Oc2cc3c(cc2-c2ccc(F)c(C(=O)O)c2)S(=O)(=O)N(C)[C@H](C2CCCCC2)CN3c2ccccc2)cc1F] |
| 412 | 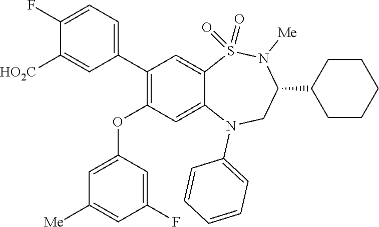 [SMILES: Cc1cc(F)cc(Oc2cc3c(cc2-c2ccc(F)c(C(=O)O)c2)S(=O)(=O)N(C)[C@H](C2CCCCC2)CN3c2ccccc2)c1] |
| 413 | 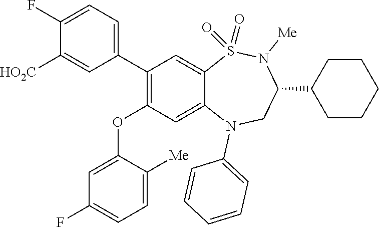 [SMILES: Cc1ccc(F)cc1Oc1cc2c(cc1-c1ccc(F)c(C(=O)O)c1)S(=O)(=O)N(C)[C@H](C1CCCCC1)CN2c1ccccc1] |
| 414 | 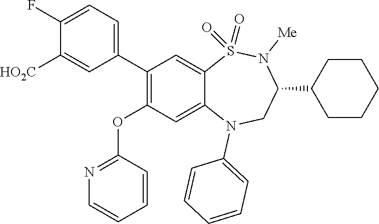 [SMILES: CN1[C@H](C2CCCCC2)CN(c2ccccc2)c2cc(Oc3ccccn3)c(-c3ccc(F)c(C(=O)O)c3)cc2S1(=O)=O] |
| 415 | 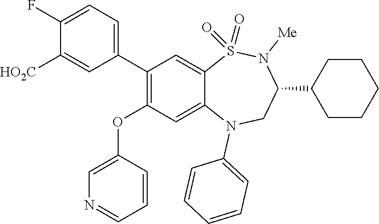 [SMILES: CN1[C@H](C2CCCCC2)CN(c2ccccc2)c2cc(Oc3cccnc3)c(-c3ccc(F)c(C(=O)O)c3)cc2S1(=O)=O] |
| 416 | 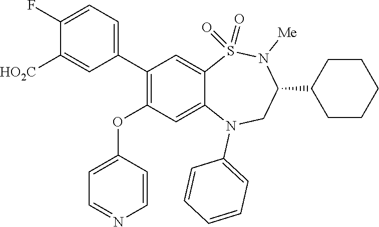 [SMILES: CN1[C@H](C2CCCCC2)CN(c2ccccc2)c2cc(Oc3ccncc3)c(-c3ccc(F)c(C(=O)O)c3)cc2S1(=O)=O] |
| 417 | 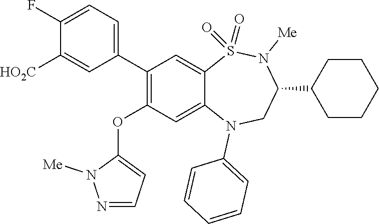 [SMILES: CN1[C@H](C2CCCCC2)CN(c2ccccc2)c2cc(Oc3ccnn3C)c(-c3ccc(F)c(C(=O)O)c3)cc2S1(=O)=O] |
| 418 | 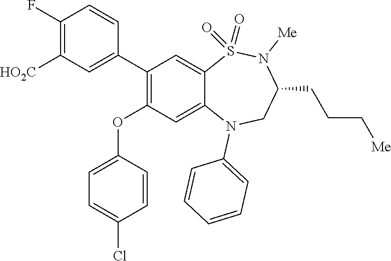 [SMILES: CCCC[C@@H]1CN(c2ccccc2)c2cc(Oc3ccc(Cl)cc3)c(-c3ccc(F)c(C(=O)O)c3)cc2S(=O)(=O)N1C] |
| 419 | 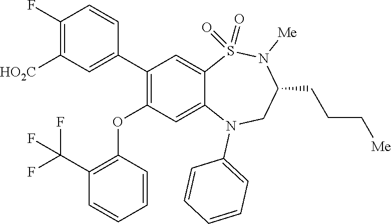 [SMILES: CCCC[C@@H]1CN(c2ccccc2)c2cc(Oc3ccccc3C(F)(F)F)c(-c3ccc(F)c(C(=O)O)c3)cc2S(=O)(=O)N1C] |
| 420 | 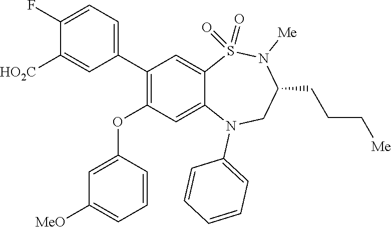 [SMILES: CCCC[C@@H]1CN(c2ccccc2)c2cc(Oc3cccc(OC)c3)c(-c3ccc(F)c(C(=O)O)c3)cc2S(=O)(=O)N1C] |
| 421 | 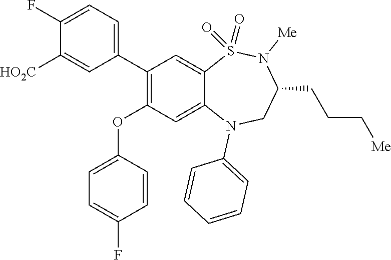 [SMILES: CCCC[C@@H]1CN(c2ccccc2)c2cc(Oc3ccc(F)cc3)c(-c3ccc(F)c(C(=O)O)c3)cc2S(=O)(=O)N1C] |
| 422 | 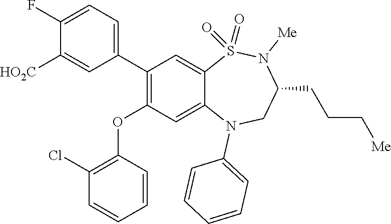 [SMILES: CCCC[C@@H]1CN(c2ccccc2)c2cc(Oc3ccccc3Cl)c(-c3ccc(F)c(C(=O)O)c3)cc2S(=O)(=O)N1C] |
| 423 | 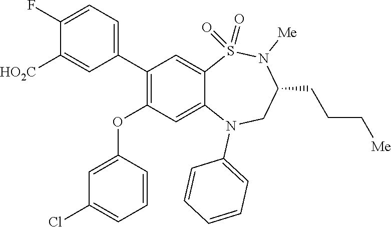 [SMILES: CCCC[C@@H]1CN(c2ccccc2)c2cc(Oc3cccc(Cl)c3)c(-c3ccc(F)c(C(=O)O)c3)cc2S(=O)(=O)N1C] |
| 424 | 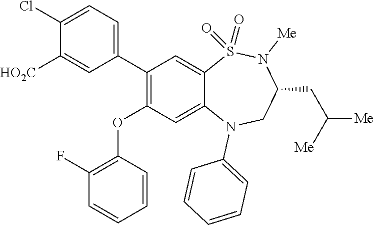 [SMILES: CC(C)C[C@@H]1CN(c2ccccc2)c2cc(Oc3ccccc3F)c(-c3ccc(Cl)c(C(=O)O)c3)cc2S(=O)(=O)N1C] |
| 425 | 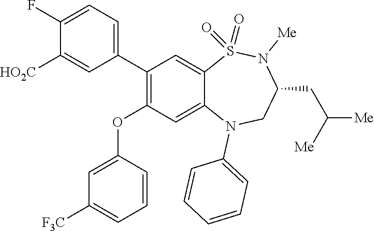 [SMILES: CC(C)C[C@@H]1CN(c2ccccc2)c2cc(Oc3cccc(C(F)(F)F)c3)c(-c3ccc(F)c(C(=O)O)c3)cc2S(=O)(=O)N1C] |
| 426 | 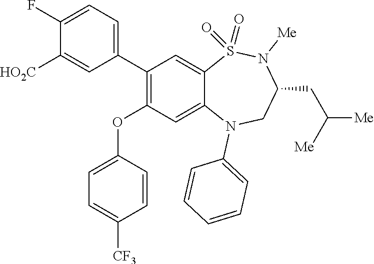 [SMILES: CC(C)C[C@@H]1CN(c2ccccc2)c2cc(Oc3ccc(C(F)(F)F)cc3)c(-c3ccc(F)c(C(=O)O)c3)cc2S(=O)(=O)N1C] |
| 427 | 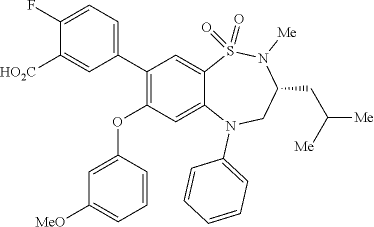 [SMILES: COc1cccc(Oc2cc3c(cc2-c2ccc(F)c(C(=O)O)c2)S(=O)(=O)N(C)[C@H](CC(C)C)CN3c2ccccc2)c1] |
| 428 | 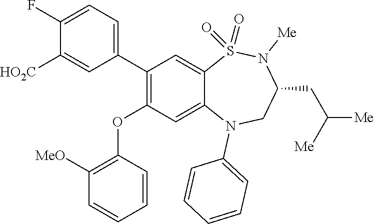 [SMILES: COc1ccccc1Oc1cc2c(cc1-c1ccc(F)c(C(=O)O)c1)S(=O)(=O)N(C)[C@H](CC(C)C)CN2c1ccccc1] |
| 429 | 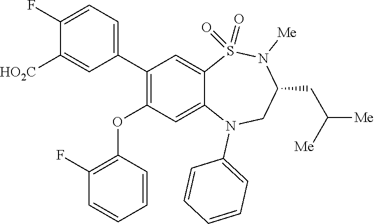 [SMILES: CC(C)C[C@@H]1CN(c2ccccc2)c2cc(Oc3ccccc3F)c(-c3ccc(F)c(C(=O)O)c3)cc2S(=O)(=O)N1C] |
| 430 | 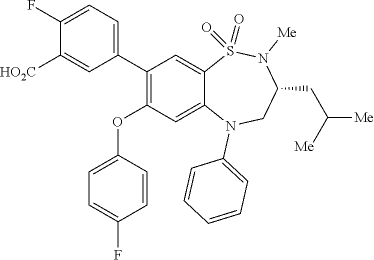 [SMILES: CC(C)C[C@@H]1CN(c2ccccc2)c2cc(Oc3ccc(F)cc3)c(-c3ccc(F)c(C(=O)O)c3)cc2S(=O)(=O)N1C] |
| 431 | 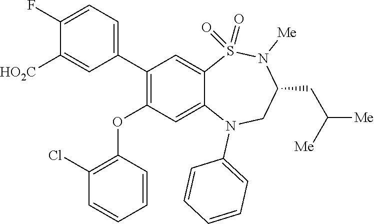 [SMILES: CC(C)C[C@@H]1CN(c2ccccc2)c2cc(Oc3ccccc3Cl)c(-c3ccc(F)c(C(=O)O)c3)cc2S(=O)(=O)N1C] |
| 432 | 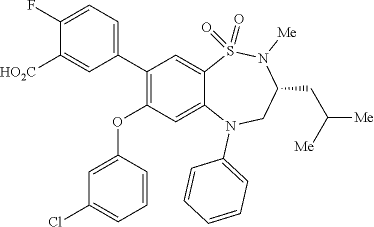 [SMILES: CC(C)C[C@@H]1CN(c2ccccc2)c2cc(Oc3cccc(Cl)c3)c(-c3ccc(F)c(C(=O)O)c3)cc2S(=O)(=O)N1C] |
| 433 | 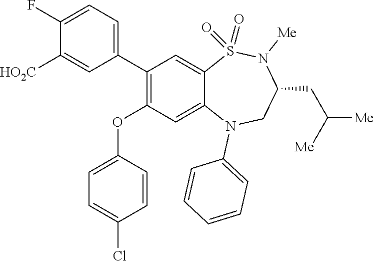 [SMILES: CC(C)C[C@@H]1CN(c2ccccc2)c2cc(Oc3ccc(Cl)cc3)c(-c3ccc(F)c(C(=O)O)c3)cc2S(=O)(=O)N1C] |
| 434 | 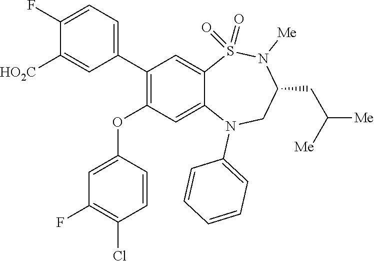 [SMILES: CC(C)C[C@@H]1CN(c2ccccc2)c2cc(Oc3ccc(Cl)c(F)c3)c(-c3ccc(F)c(C(=O)O)c3)cc2S(=O)(=O)N1C] |
| 435 | 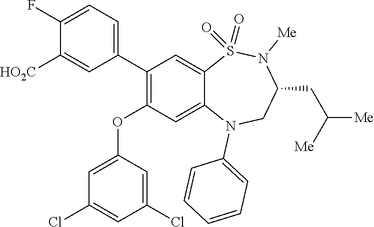 [SMILES: CC(C)C[C@@H]1CN(c2ccccc2)c2cc(Oc3cc(Cl)cc(Cl)c3)c(-c3ccc(F)c(C(=O)O)c3)cc2S(=O)(=O)N1C] |
| 436 | 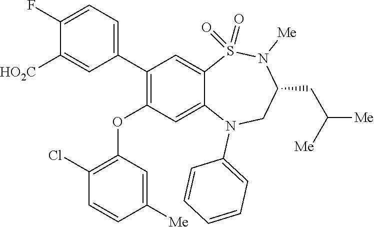 [SMILES: Cc1ccc(Cl)c(Oc2cc3c(cc2-c2ccc(F)c(C(=O)O)c2)S(=O)(=O)N(C)[C@H](CC(C)C)CN3c2ccccc2)c1] |
| 437 | 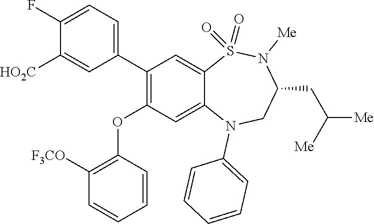 [SMILES: CC(C)C[C@@H]1CN(c2ccccc2)c2cc(Oc3ccccc3OC(F)(F)F)c(-c3ccc(F)c(C(=O)O)c3)cc2S(=O)(=O)N1C] |
| 438 | 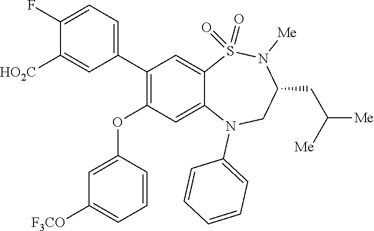 [SMILES: CC(C)C[C@@H]1CN(c2ccccc2)c2cc(Oc3cccc(OC(F)(F)F)c3)c(-c3ccc(F)c(C(=O)O)c3)cc2S(=O)(=O)N1C] |
| 439 | 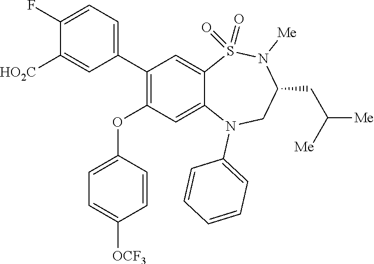 [SMILES: CC(C)C[C@@H]1CN(c2ccccc2)c2cc(Oc3ccc(OC(F)(F)F)cc3)c(-c3ccc(F)c(C(=O)O)c3)cc2S(=O)(=O)N1C] |
| 440 | 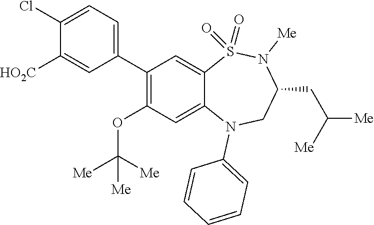 [SMILES: CC(C)C[C@@H]1CN(c2ccccc2)c2cc(OC(C)(C)C)c(-c3ccc(Cl)c(C(=O)O)c3)cc2S(=O)(=O)N1C] |
| 441 | 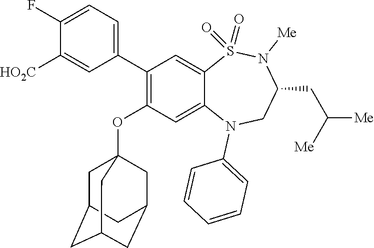 [SMILES: CC(C)C[C@@H]1CN(c2ccccc2)c2cc(OC34CC5CC(CC(C5)C3)C4)c(-c3ccc(F)c(C(=O)O)c3)cc2S(=O)(=O)N1C] |
| 442 | 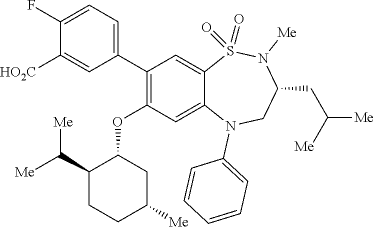 [SMILES: CC(C)C[C@@H]1CN(c2ccccc2)c2cc(O[C@@H]3C[C@H](C)CC[C@H]3C(C)C)c(-c3ccc(F)c(C(=O)O)c3)cc2S(=O)(=O)N1C] |
| 443 | 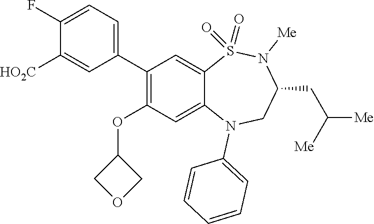 [SMILES: CC(C)C[C@@H]1CN(c2ccccc2)c2cc(OC3COC3)c(-c3ccc(F)c(C(=O)O)c3)cc2S(=O)(=O)N1C] |
| 444 | 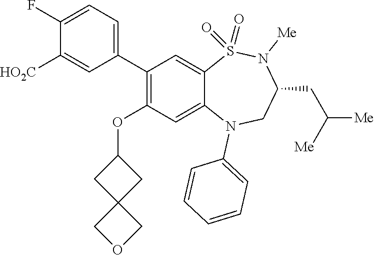 [SMILES: CC(C)C[C@@H]1CN(c2ccccc2)c2cc(OC3CC4(COC4)C3)c(-c3ccc(F)c(C(=O)O)c3)cc2S(=O)(=O)N1C] |
| 445 | 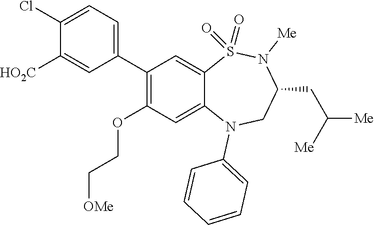 [SMILES: COCCOc1cc2c(cc1-c1ccc(Cl)c(C(=O)O)c1)S(=O)(=O)N(C)[C@H](CC(C)C)CN2c1ccccc1] |
| 446 | 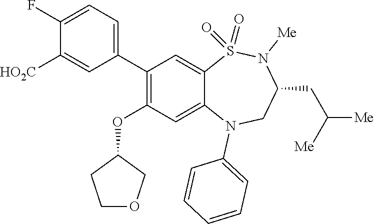 [SMILES: CC(C)C[C@@H]1CN(c2ccccc2)c2cc(O[C@H]3CCOC3)c(-c3ccc(F)c(C(=O)O)c3)cc2S(=O)(=O)N1C] |
| 447 | 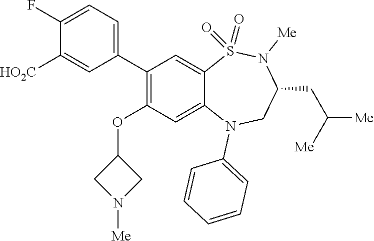 [SMILES: CC(C)C[C@@H]1CN(c2ccccc2)c2cc(OC3CN(C)C3)c(-c3ccc(F)c(C(=O)O)c3)cc2S(=O)(=O)N1C] |
| 448 | 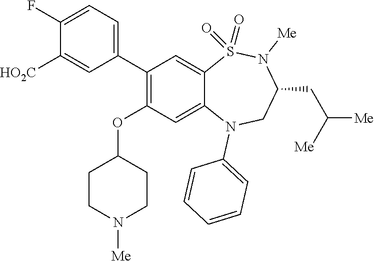 [SMILES: CC(C)C[C@@H]1CN(c2ccccc2)c2cc(OC3CCN(C)CC3)c(-c3ccc(F)c(C(=O)O)c3)cc2S(=O)(=O)N1C] |
| 449 | 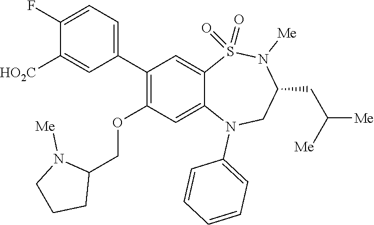 [SMILES: CC(C)C[C@@H]1CN(c2ccccc2)c2cc(OCC3CCCN3C)c(-c3ccc(F)c(C(=O)O)c3)cc2S(=O)(=O)N1C] |
| 450 | 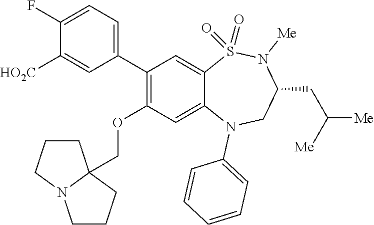 [SMILES: CC(C)C[C@@H]1CN(c2ccccc2)c2cc(OCC34CCCN3CCC4)c(-c3ccc(F)c(C(=O)O)c3)cc2S(=O)(=O)N1C] |
| 451 | 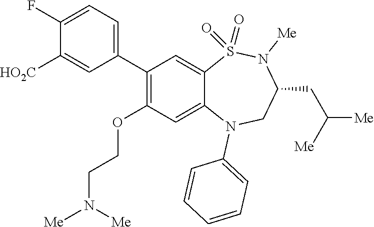 [SMILES: CC(C)C[C@@H]1CN(c2ccccc2)c2cc(OCCN(C)C)c(-c3ccc(F)c(C(=O)O)c3)cc2S(=O)(=O)N1C] |
| 452 | 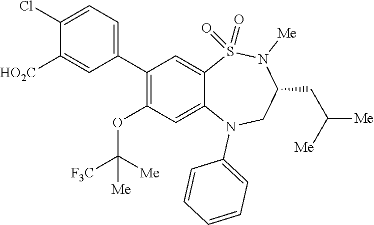 [SMILES: CC(C)C[C@@H]1CN(c2ccccc2)c2cc(OC(C)(C)C(F)(F)F)c(-c3ccc(Cl)c(C(=O)O)c3)cc2S(=O)(=O)N1C] |
| 453 | 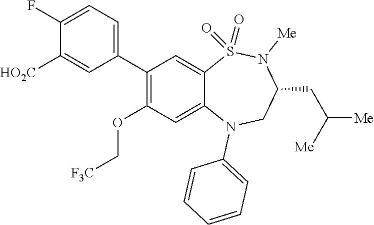 [SMILES: CC(C)C[C@@H]1CN(c2ccccc2)c2cc(OCC(F)(F)F)c(-c3ccc(F)c(C(=O)O)c3)cc2S(=O)(=O)N1C] |
| 454 | 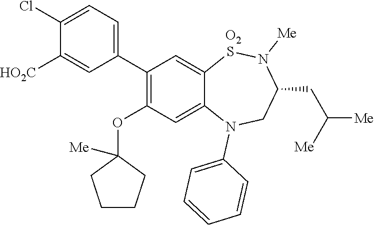 |
| 455 | 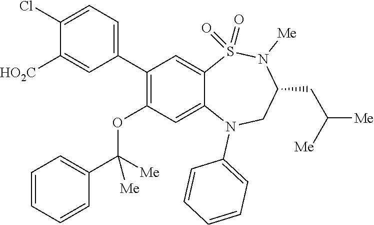 [SMILES: CC(C)C[C@@H]1CN(c2ccccc2)c2cc(OC(C)(C)c3ccccc3)c(-c3ccc(Cl)c(C(=O)O)c3)cc2S(=O)(=O)N1C] |
| 456 | 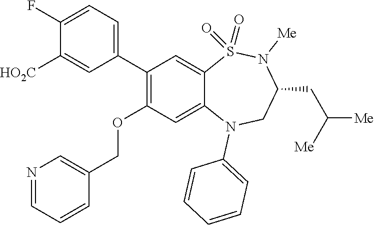 [SMILES: CC(C)C[C@@H]1CN(c2ccccc2)c2cc(OCc3cccnc3)c(-c3ccc(F)c(C(=O)O)c3)cc2S(=O)(=O)N1C] |
| 457 | 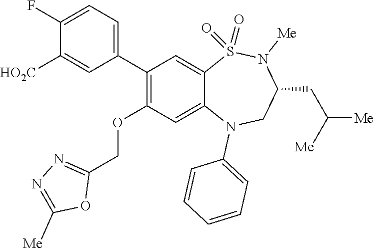 [SMILES: Cc1nnc(COc2cc3c(cc2-c2ccc(F)c(C(=O)O)c2)S(=O)(=O)N(C)[C@H](CC(C)C)CN3c2ccccc2)o1] |
| 458 | 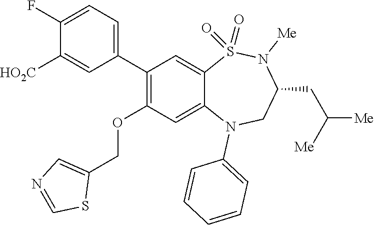 [SMILES: CC(C)C[C@@H]1CN(c2ccccc2)c2cc(OCc3cncs3)c(-c3ccc(F)c(C(=O)O)c3)cc2S(=O)(=O)N1C] |
| 459 | 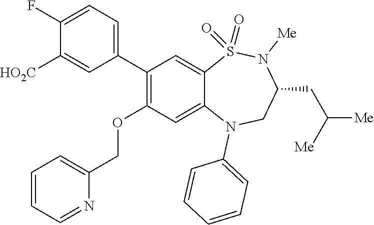 [SMILES: CC(C)C[C@@H]1CN(c2ccccc2)c2cc(OCc3ccccn3)c(-c3ccc(F)c(C(=O)O)c3)cc2S(=O)(=O)N1C] |
| 460 | 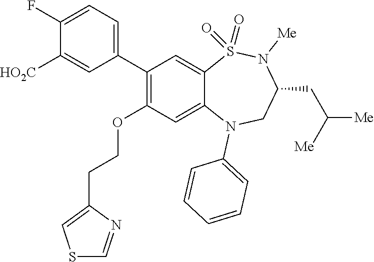 [SMILES: CC(C)C[C@@H]1CN(c2ccccc2)c2cc(OCCc3cscn3)c(-c3ccc(F)c(C(=O)O)c3)cc2S(=O)(=O)N1C] |
| 461 | 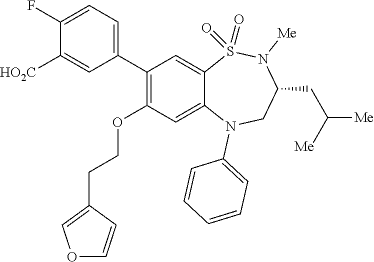 [SMILES: CC(C)C[C@@H]1CN(c2ccccc2)c2cc(OCCc3ccoc3)c(-c3ccc(F)c(C(=O)O)c3)cc2S(=O)(=O)N1C] |
| 462 | 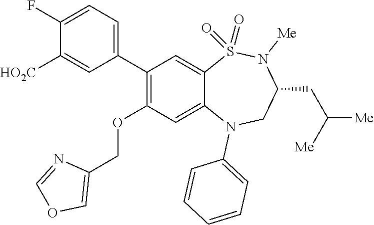 [SMILES: CC(C)C[C@@H]1CN(c2ccccc2)c2cc(OCc3cocn3)c(-c3ccc(F)c(C(=O)O)c3)cc2S(=O)(=O)N1C] |
| 463 | 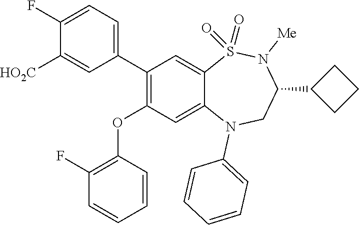 [SMILES: CN1[C@H](C2CCC2)CN(c2ccccc2)c2cc(Oc3ccccc3F)c(-c3ccc(F)c(C(=O)O)c3)cc2S1(=O)=O] |
| 464 | 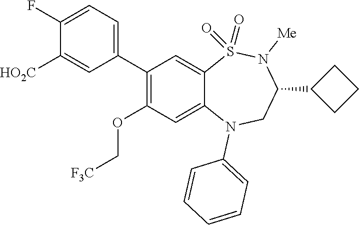 [SMILES: CN1[C@H](C2CCC2)CN(c2ccccc2)c2cc(OCC(F)(F)F)c(-c3ccc(F)c(C(=O)O)c3)cc2S1(=O)=O] |
| 465 | 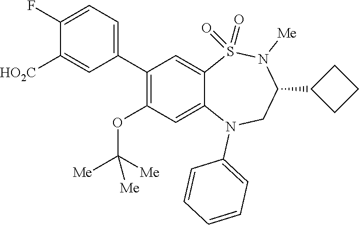 [SMILES: CN1[C@H](C2CCC2)CN(c2ccccc2)c2cc(OC(C)(C)C)c(-c3ccc(F)c(C(=O)O)c3)cc2S1(=O)=O] |
| 466 | 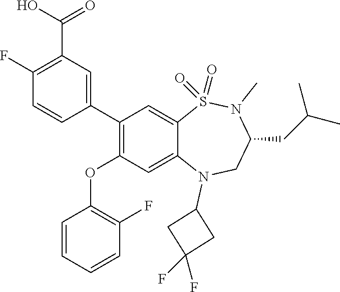 [SMILES: CC(C)C[C@@H]1CN(C2CC(F)(F)C2)c2cc(Oc3ccccc3F)c(-c3ccc(F)c(C(=O)O)c3)cc2S(=O)(=O)N1C] |
| 467 | 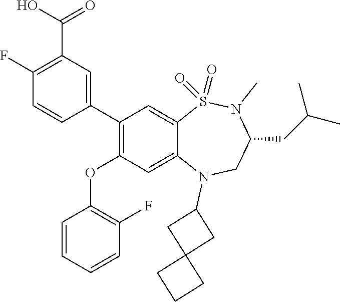 [SMILES: CC(C)C[C@@H]1CN(C2CC3(CCC3)C2)c2cc(Oc3ccccc3F)c(-c3ccc(F)c(C(=O)O)c3)cc2S(=O)(=O)N1C] |
| 468 | 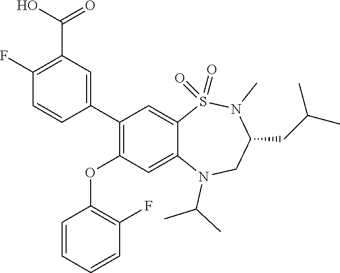 [SMILES: CC(C)C[C@@H]1CN(C(C)C)c2cc(Oc3ccccc3F)c(-c3ccc(F)c(C(=O)O)c3)cc2S(=O)(=O)N1C] |
| 469 | 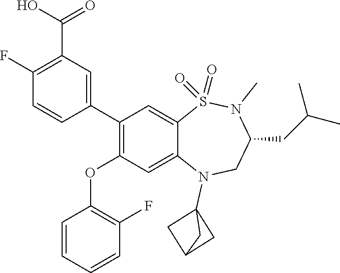 [SMILES: CC(C)C[C@@H]1CN(C23CC(C2)C3)c2cc(Oc3ccccc3F)c(-c3ccc(F)c(C(=O)O)c3)cc2S(=O)(=O)N1C] |
| 470 | 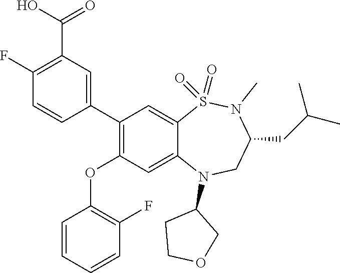 [SMILES: CC(C)C[C@@H]1CN([C@@H]2CCOC2)c2cc(Oc3ccccc3F)c(-c3ccc(F)c(C(=O)O)c3)cc2S(=O)(=O)N1C] |
| 471 | 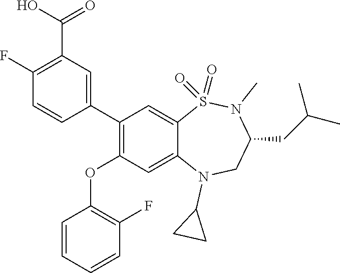 [SMILES: CC(C)C[C@@H]1CN(C2CC2)c2cc(Oc3ccccc3F)c(-c3ccc(F)c(C(=O)O)c3)cc2S(=O)(=O)N1C] |
| 472 | 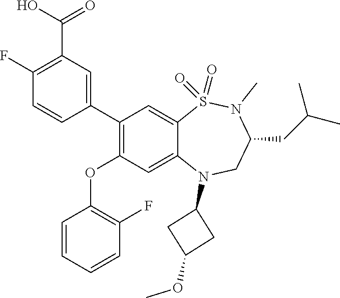 [SMILES: CO[C@H]1C[C@H](N2C[C@@H](CC(C)C)N(C)S(=O)(=O)c3cc(-c4ccc(F)c(C(=O)O)c4)c(Oc4ccccc4F)cc32)C1] |
| 473 | 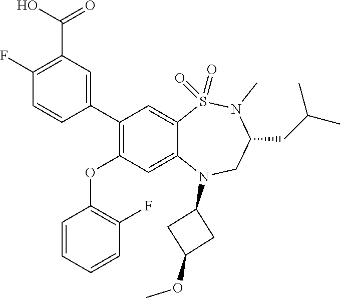 [SMILES: CO[C@H]1C[C@@H](N2C[C@@H](CC(C)C)N(C)S(=O)(=O)c3cc(-c4ccc(F)c(C(=O)O)c4)c(Oc4ccccc4F)cc32)C1] |
| 474 | 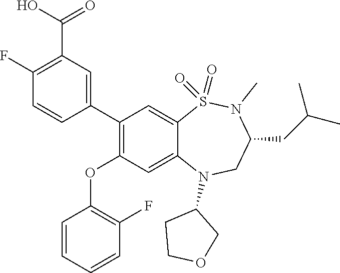 [SMILES: CC(C)C[C@@H]1CN([C@H]2CCOC2)c2cc(Oc3ccccc3F)c(-c3ccc(F)c(C(=O)O)c3)cc2S(=O)(=O)N1C] |
| 475 | 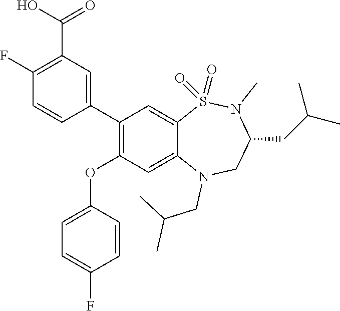 [SMILES: CC(C)C[C@@H]1CN(CC(C)C)c2cc(Oc3ccc(F)cc3)c(-c3ccc(F)c(C(=O)O)c3)cc2S(=O)(=O)N1C] |
| 476 | 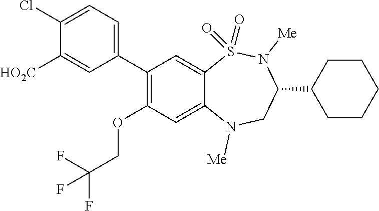 [SMILES: CN1C[C@@H](C2CCCCC2)N(C)S(=O)(=O)c2cc(-c3ccc(Cl)c(C(=O)O)c3)c(OCC(F)(F)F)cc21] |
| 477 | 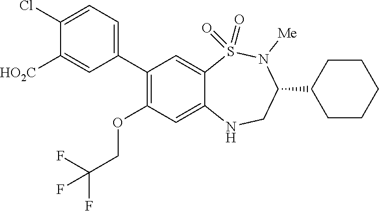 [SMILES: CN1[C@H](C2CCCCC2)CNc2cc(OCC(F)(F)F)c(-c3ccc(Cl)c(C(=O)O)c3)cc2S1(=O)=O] |
| 478 | 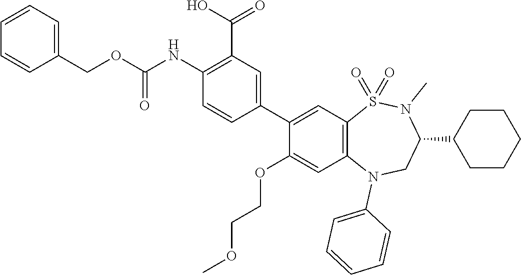 [SMILES: COCCOc1cc2c(cc1-c1ccc(NC(=O)OCc3ccccc3)c(C(=O)O)c1)S(=O)(=O)N(C)[C@H](C1CCCCC1)CN2c1ccccc1] |
| 479 | 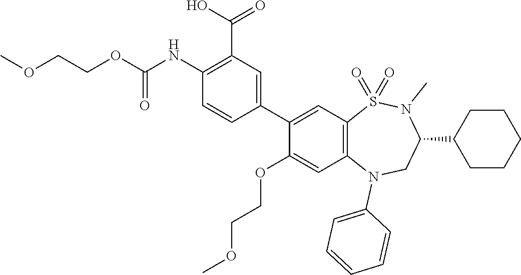 [SMILES: COCCOC(=O)Nc1ccc(-c2cc3c(cc2OCCOC)N(c2ccccc2)C[C@@H](C2CCCCC2)N(C)S3(=O)=O)cc1C(=O)O] |
| 480 | 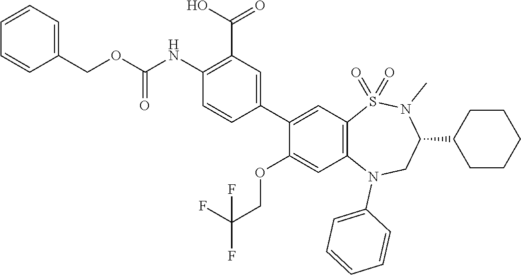 [SMILES: CN1[C@H](C2CCCCC2)CN(c2ccccc2)c2cc(OCC(F)(F)F)c(-c3ccc(NC(=O)OCc4ccccc4)c(C(=O)O)c3)cc2S1(=O)=O] |
| 481 | 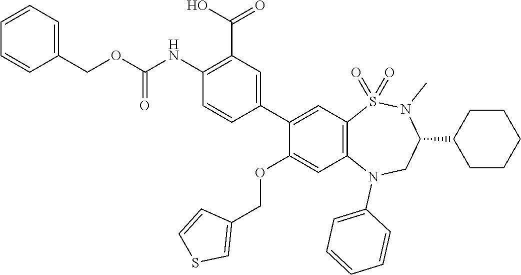 [SMILES: CN1[C@H](C2CCCCC2)CN(c2ccccc2)c2cc(OCc3ccsc3)c(-c3ccc(NC(=O)OCc4ccccc4)c(C(=O)O)c3)cc2S1(=O)=O] |
| 482 | 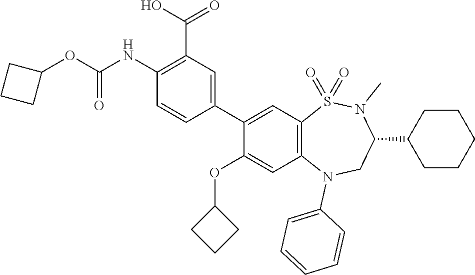 [SMILES: CN1[C@H](C2CCCCC2)CN(c2ccccc2)c2cc(OC3CCC3)c(-c3ccc(NC(=O)OC4CCC4)c(C(=O)O)c3)cc2S1(=O)=O] |
| 483 | 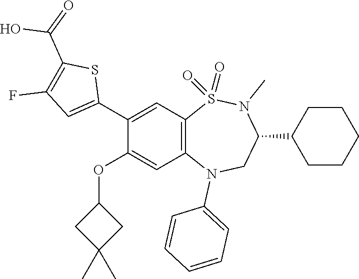 [SMILES: CN1[C@H](C2CCCCC2)CN(c2ccccc2)c2cc(OC3CC(C)(C)C3)c(-c3cc(F)c(C(=O)O)s3)cc2S1(=O)=O] |
| 484 | 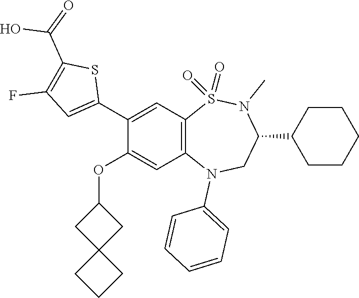 [SMILES: CN1[C@H](C2CCCCC2)CN(c2ccccc2)c2cc(OC3CC4(CCC4)C3)c(-c3cc(F)c(C(=O)O)s3)cc2S1(=O)=O] |
| 485 | 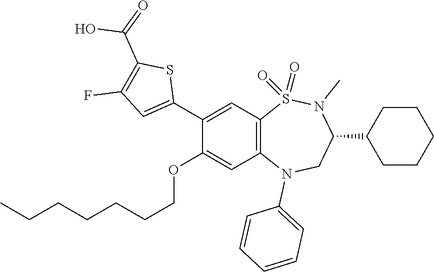 [SMILES: CCCCCCCOc1cc2c(cc1-c1cc(F)c(C(=O)O)s1)S(=O)(=O)N(C)[C@H](C1CCCCC1)CN2c1ccccc1] |
| 486 | 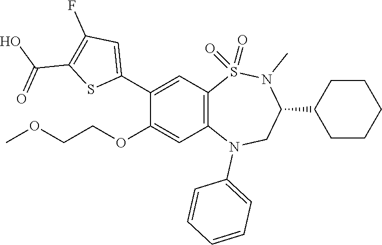 [SMILES: COCCOc1cc2c(cc1-c1cc(F)c(C(=O)O)s1)S(=O)(=O)N(C)[C@H](C1CCCCC1)CN2c1ccccc1] |
| 487 | 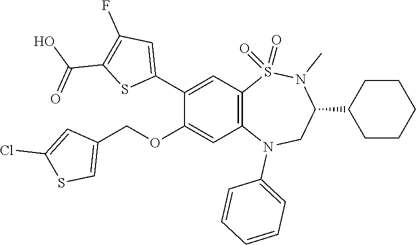 [SMILES: CN1[C@H](C2CCCCC2)CN(c2ccccc2)c2cc(OCc3csc(Cl)c3)c(-c3cc(F)c(C(=O)O)s3)cc2S1(=O)=O] |
| 488 | 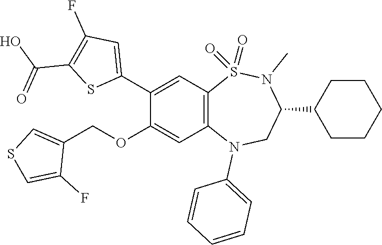 [SMILES: CN1[C@H](C2CCCCC2)CN(c2ccccc2)c2cc(OCc3cscc3F)c(-c3cc(F)c(C(=O)O)s3)cc2S1(=O)=O] |
| 489 | 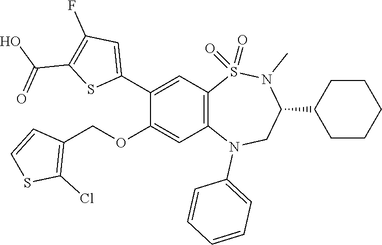 [SMILES: CN1[C@H](C2CCCCC2)CN(c2ccccc2)c2cc(OCc3ccsc3Cl)c(-c3cc(F)c(C(=O)O)s3)cc2S1(=O)=O] |
| 490 | 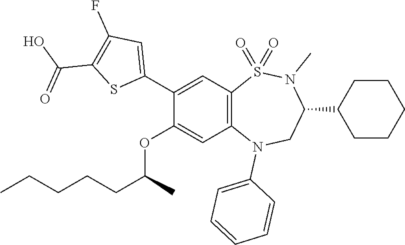 [SMILES: CCCCC[C@H](C)Oc1cc2c(cc1-c1cc(F)c(C(=O)O)s1)S(=O)(=O)N(C)[C@H](C1CCCCC1)CN2c1ccccc1] |
| 491 | 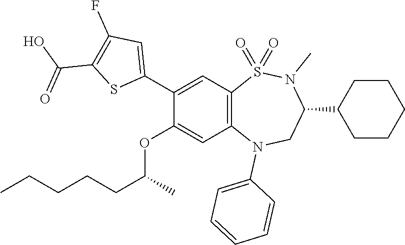 [SMILES: CCCCC[C@@H](C)Oc1cc2c(cc1-c1cc(F)c(C(=O)O)s1)S(=O)(=O)N(C)[C@H](C1CCCCC1)CN2c1ccccc1] |
| 492 | 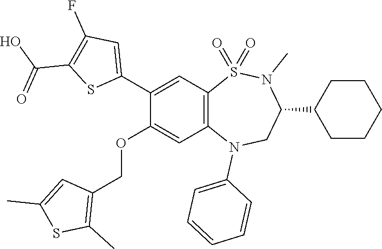 [SMILES: Cc1cc(COc2cc3c(cc2-c2cc(F)c(C(=O)O)s2)S(=O)(=O)N(C)[C@H](C2CCCCC2)CN3c2ccccc2)c(C)s1] |
| 493 | 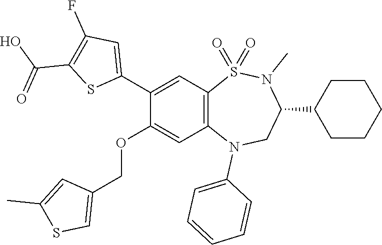 [SMILES: Cc1cc(COc2cc3c(cc2-c2cc(F)c(C(=O)O)s2)S(=O)(=O)N(C)[C@H](C2CCCCC2)CN3c2ccccc2)cs1] |
| 494 | 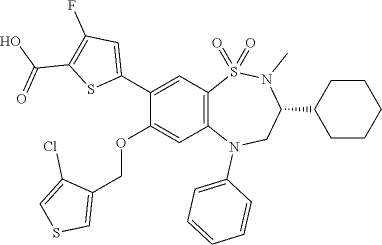 [SMILES: CN1[C@H](C2CCCCC2)CN(c2ccccc2)c2cc(OCc3cscc3Cl)c(-c3cc(F)c(C(=O)O)s3)cc2S1(=O)=O] |
| 495 | 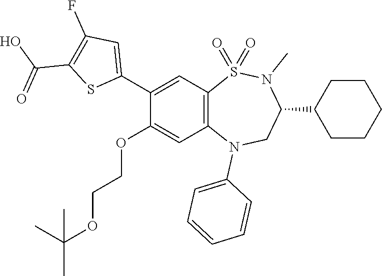 [SMILES: CN1[C@H](C2CCCCC2)CN(c2ccccc2)c2cc(OCCOC(C)(C)C)c(-c3cc(F)c(C(=O)O)s3)cc2S1(=O)=O] |
| 496 | 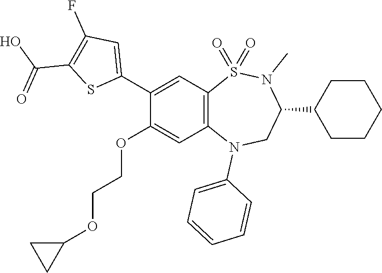 [SMILES: CN1[C@H](C2CCCCC2)CN(c2ccccc2)c2cc(OCCOC3CC3)c(-c3cc(F)c(C(=O)O)s3)cc2S1(=O)=O] |
| 497 | 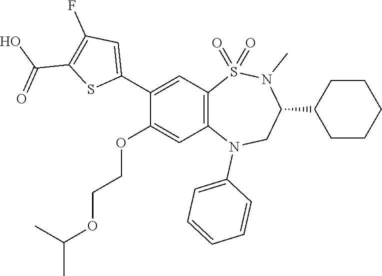 [SMILES: CC(C)OCCOc1cc2c(cc1-c1cc(F)c(C(=O)O)s1)S(=O)(=O)N(C)[C@H](C1CCCCC1)CN2c1ccccc1] |
| 498 | 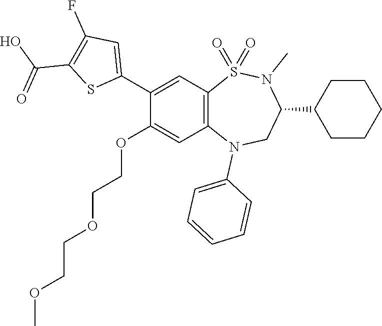 [SMILES: COCCOCCOc1cc2c(cc1-c1cc(F)c(C(=O)O)s1)S(=O)(=O)N(C)[C@H](C1CCCCC1)CN2c1ccccc1] |
| 499 | 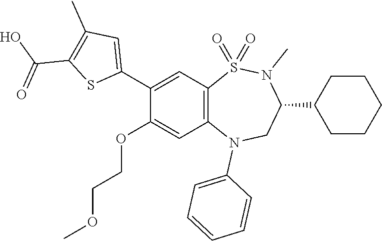 [SMILES: COCCOc1cc2c(cc1-c1cc(C)c(C(=O)O)s1)S(=O)(=O)N(C)[C@H](C1CCCCC1)CN2c1ccccc1] |
| 500 | 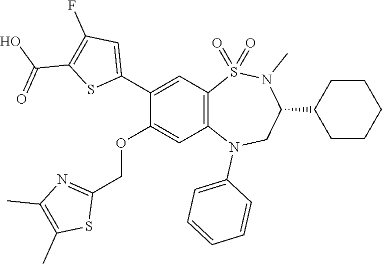 [SMILES: Cc1nc(COc2cc3c(cc2-c2cc(F)c(C(=O)O)s2)S(=O)(=O)N(C)[C@H](C2CCCCC2)CN3c2ccccc2)sc1C] |
| 501 | 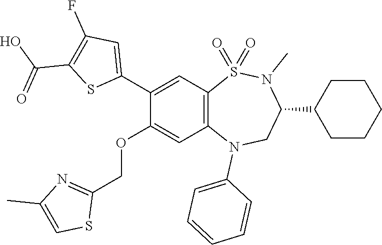 [SMILES: Cc1csc(COc2cc3c(cc2-c2cc(F)c(C(=O)O)s2)S(=O)(=O)N(C)[C@H](C2CCCCC2)CN3c2ccccc2)n1] |
| 502 | 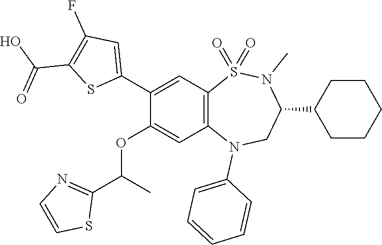 [SMILES: CC(Oc1cc2c(cc1-c1cc(F)c(C(=O)O)s1)S(=O)(=O)N(C)[C@H](C1CCCCC1)CN2c1ccccc1)c1nccs1] |
| 503 | 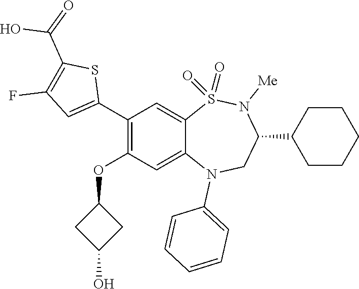 [SMILES: CN1[C@H](C2CCCCC2)CN(c2ccccc2)c2cc(O[C@H]3C[C@H](O)C3)c(-c3cc(F)c(C(=O)O)s3)cc2S1(=O)=O] |
| 504 | 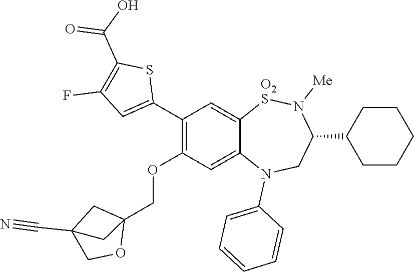 [SMILES: CN1[C@H](C2CCCCC2)CN(c2ccccc2)c2cc(OCC34CC(C#N)(CO3)C4)c(-c3cc(F)c(C(=O)O)s3)cc2S1(=O)=O] |
| 505 | 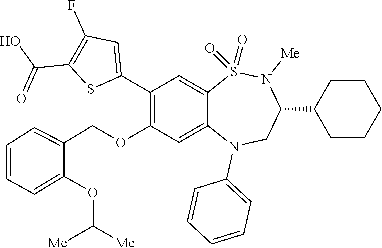 [SMILES: CC(C)Oc1ccccc1COc1cc2c(cc1-c1cc(F)c(C(=O)O)s1)S(=O)(=O)N(C)[C@H](C1CCCCC1)CN2c1ccccc1] |
| 506 | 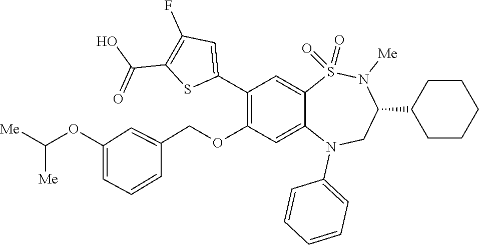 [SMILES: CC(C)Oc1cccc(COc2cc3c(cc2-c2cc(F)c(C(=O)O)s2)S(=O)(=O)N(C)[C@H](C2CCCCC2)CN3c2ccccc2)c1] |
| 507 | 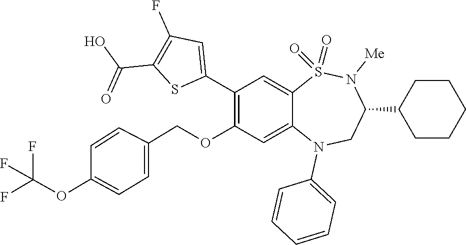 [SMILES: CN1[C@H](C2CCCCC2)CN(c2ccccc2)c2cc(OCc3ccc(OC(F)(F)F)cc3)c(-c3cc(F)c(C(=O)O)s3)cc2S1(=O)=O] |
| 508 | 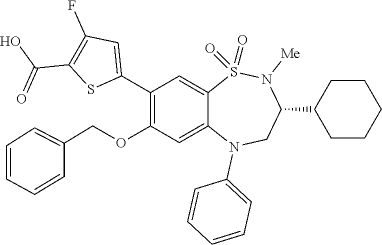 [SMILES: CN1[C@H](C2CCCCC2)CN(c2ccccc2)c2cc(OCc3ccccc3)c(-c3cc(F)c(C(=O)O)s3)cc2S1(=O)=O] |
| 509 | 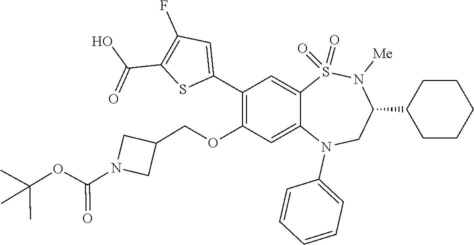 [SMILES: CN1[C@H](C2CCCCC2)CN(c2ccccc2)c2cc(OCC3CN(C(=O)OC(C)(C)C)C3)c(-c3cc(F)c(C(=O)O)s3)cc2S1(=O)=O] |
| 510 | 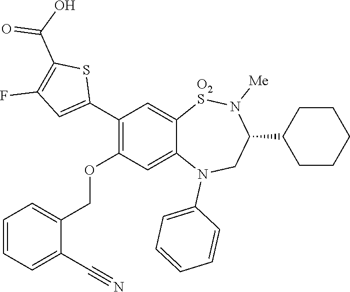 [SMILES: CN1[C@H](C2CCCCC2)CN(c2ccccc2)c2cc(OCc3ccccc3C#N)c(-c3cc(F)c(C(=O)O)s3)cc2S1(=O)=O] |
| 511 | 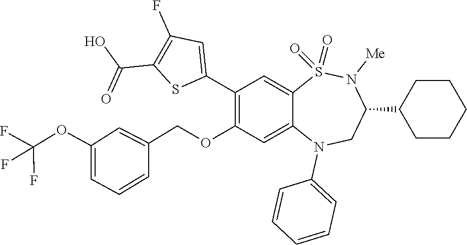 [SMILES: CN1[C@H](C2CCCCC2)CN(c2ccccc2)c2cc(OCc3cccc(OC(F)(F)F)c3)c(-c3cc(F)c(C(=O)O)s3)cc2S1(=O)=O] |
| 512 | 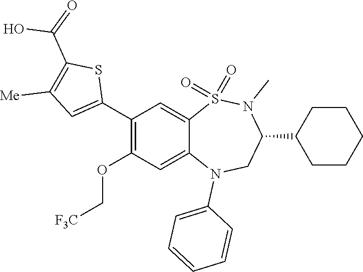 [SMILES: Cc1cc(-c2cc3c(cc2OCC(F)(F)F)N(c2ccccc2)C[C@@H](C2CCCCC2)N(C)S3(=O)=O)sc1C(=O)O] |
| 513 | 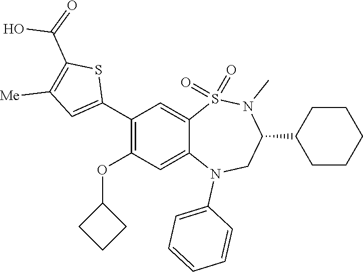 [SMILES: Cc1cc(-c2cc3c(cc2OC2CCC2)N(c2ccccc2)C[C@@H](C2CCCCC2)N(C)S3(=O)=O)sc1C(=O)O] |
| 514 | 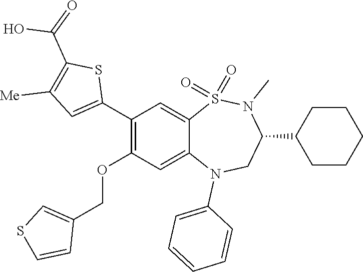 [SMILES: Cc1cc(-c2cc3c(cc2OCc2ccsc2)N(c2ccccc2)C[C@@H](C2CCCCC2)N(C)S3(=O)=O)sc1C(=O)O] |
| 515 | 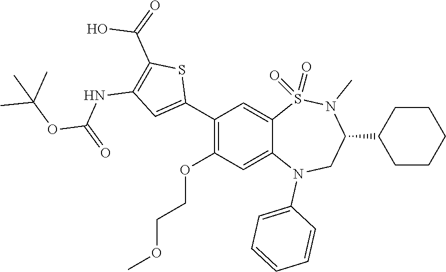 [SMILES: COCCOc1cc2c(cc1-c1cc(NC(=O)OC(C)(C)C)c(C(=O)O)s1)S(=O)(=O)N(C)[C@H](C1CCCCC1)CN2c1ccccc1] |
| 516 | 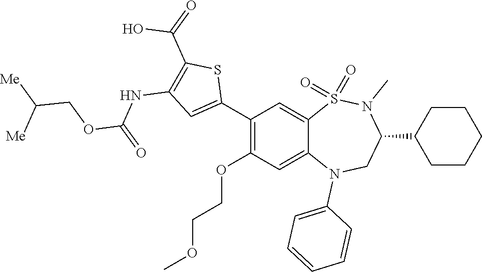 [SMILES: COCCOc1cc2c(cc1-c1cc(NC(=O)OCC(C)C)c(C(=O)O)s1)S(=O)(=O)N(C)[C@H](C1CCCCC1)CN2c1ccccc1] |
| 517 | 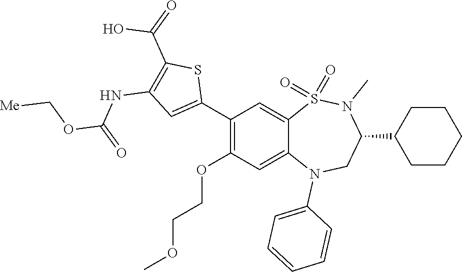 [SMILES: CCOC(=O)Nc1cc(-c2cc3c(cc2OCCOC)N(c2ccccc2)C[C@@H](C2CCCCC2)N(C)S3(=O)=O)sc1C(=O)O] |
| 518 | 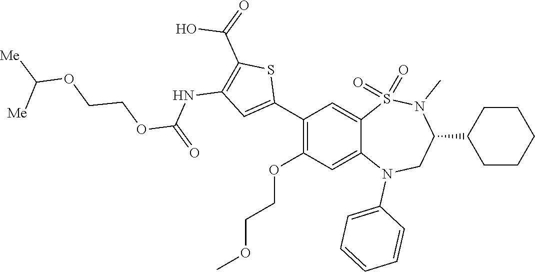 [SMILES: COCCOc1cc2c(cc1-c1cc(NC(=O)OCCOC(C)C)c(C(=O)O)s1)S(=O)(=O)N(C)[C@H](C1CCCCC1)CN2c1ccccc1] |
| 519 | 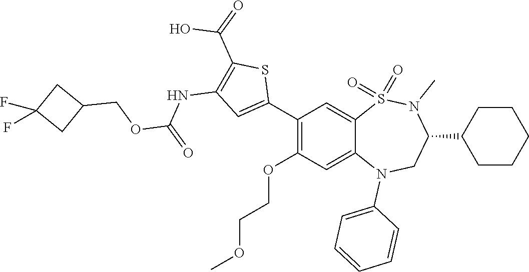 [SMILES: COCCOc1cc2c(cc1-c1cc(NC(=O)OCC3CC(F)(F)C3)c(C(=O)O)s1)S(=O)(=O)N(C)[C@H](C1CCCCC1)CN2c1ccccc1] |
| 520 | 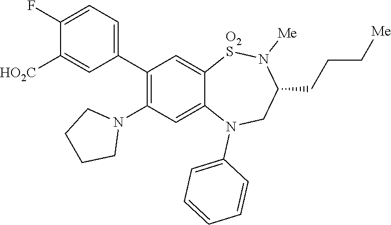 [SMILES: CCCC[C@@H]1CN(c2ccccc2)c2cc(N3CCCC3)c(-c3ccc(F)c(C(=O)O)c3)cc2S(=O)(=O)N1C] |
| 521 | 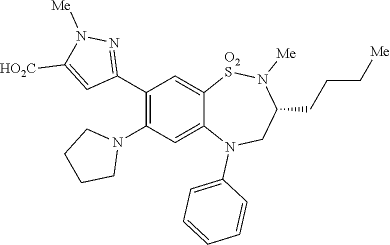 [SMILES: CCCC[C@@H]1CN(c2ccccc2)c2cc(N3CCCC3)c(-c3cc(C(=O)O)n(C)n3)cc2S(=O)(=O)N1C] |
| 522 | 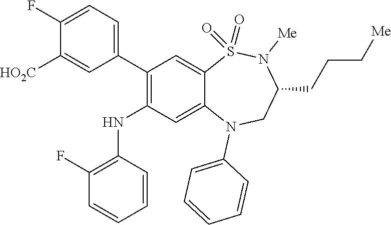 [SMILES: CCCC[C@@H]1CN(c2ccccc2)c2cc(Nc3ccccc3F)c(-c3ccc(F)c(C(=O)O)c3)cc2S(=O)(=O)N1C] |
| 523 | 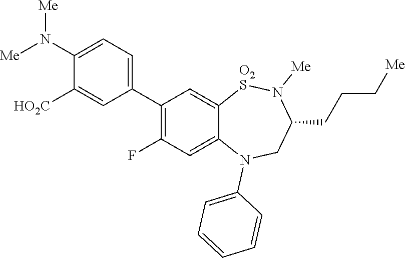 [SMILES: CCCC[C@@H]1CN(c2ccccc2)c2cc(F)c(-c3ccc(N(C)C)c(C(=O)O)c3)cc2S(=O)(=O)N1C] |
| 524 | 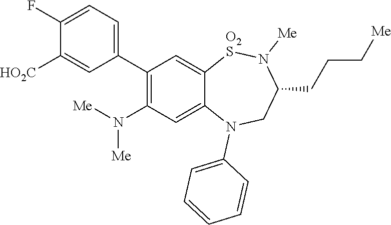 [SMILES: CCCC[C@@H]1CN(c2ccccc2)c2cc(N(C)C)c(-c3ccc(F)c(C(=O)O)c3)cc2S(=O)(=O)N1C] |
| 525 | 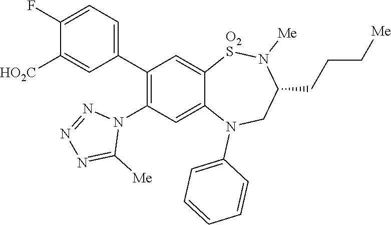 [SMILES: CCCC[C@@H]1CN(c2ccccc2)c2cc(-n3nnnc3C)c(-c3ccc(F)c(C(=O)O)c3)cc2S(=O)(=O)N1C] |
| 526 | 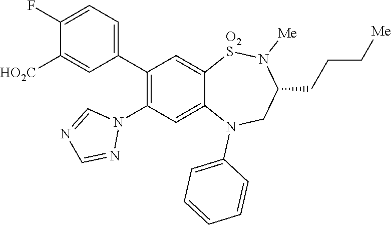 [SMILES: CCCC[C@@H]1CN(c2ccccc2)c2cc(-n3cncn3)c(-c3ccc(F)c(C(=O)O)c3)cc2S(=O)(=O)N1C] |
| 527 | 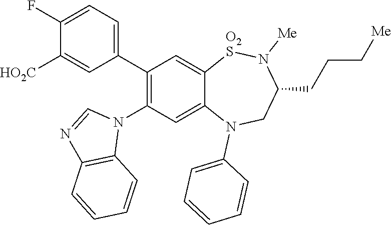 [SMILES: CCCC[C@@H]1CN(c2ccccc2)c2cc(-n3cnc4ccccc43)c(-c3ccc(F)c(C(=O)O)c3)cc2S(=O)(=O)N1C] |
| 528 | 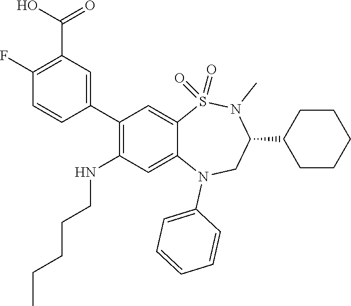 [SMILES: CCCCCNc1cc2c(cc1-c1ccc(F)c(C(=O)O)c1)S(=O)(=O)N(C)[C@H](C1CCCCC1)CN2c1ccccc1] |
| 529 | 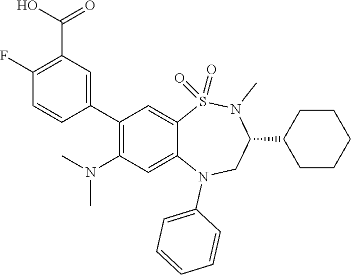 [SMILES: CN(C)c1cc2c(cc1-c1ccc(F)c(C(=O)O)c1)S(=O)(=O)N(C)[C@H](C1CCCCC1)CN2c1ccccc1] |
| 530 | 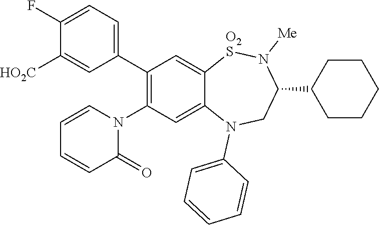 [SMILES: CN1[C@H](C2CCCCC2)CN(c2ccccc2)c2cc(-n3ccccc3=O)c(-c3ccc(F)c(C(=O)O)c3)cc2S1(=O)=O] |
| 531 | 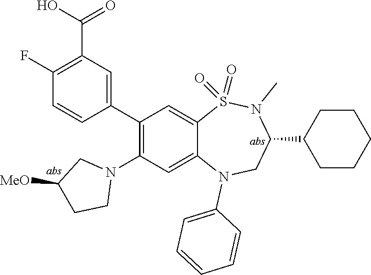 [SMILES: CO[C@@H]1CCN(c2cc3c(cc2-c2ccc(F)c(C(=O)O)c2)S(=O)(=O)N(C)[C@H](C2CCCCC2)CN3c2ccccc2)C1] |
| 532 | 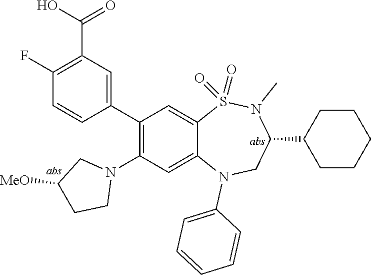 [SMILES: CO[C@H]1CCN(c2cc3c(cc2-c2ccc(F)c(C(=O)O)c2)S(=O)(=O)N(C)[C@H](C2CCCCC2)CN3c2ccccc2)C1] |
| 533 | 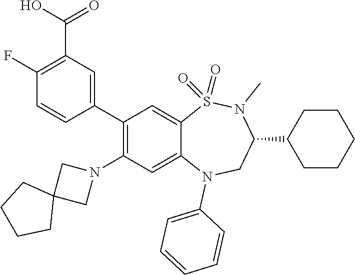 [SMILES: CN1[C@H](C2CCCCC2)CN(c2ccccc2)c2cc(N3CC4(CCCC4)C3)c(-c3ccc(F)c(C(=O)O)c3)cc2S1(=O)=O] |
| 534 | 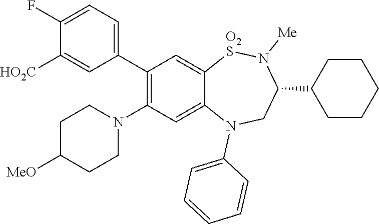 [SMILES: COC1CCN(c2cc3c(cc2-c2ccc(F)c(C(=O)O)c2)S(=O)(=O)N(C)[C@H](C2CCCCC2)CN3c2ccccc2)CC1] |
| 535 | 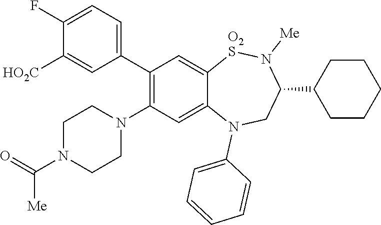 [SMILES: CC(=O)N1CCN(c2cc3c(cc2-c2ccc(F)c(C(=O)O)c2)S(=O)(=O)N(C)[C@H](C2CCCCC2)CN3c2ccccc2)CC1] |
| 536 | 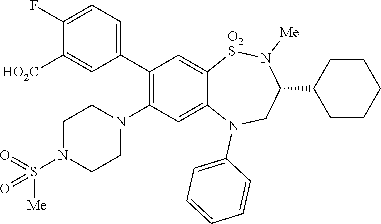 [SMILES: CN1[C@H](C2CCCCC2)CN(c2ccccc2)c2cc(N3CCN(S(C)(=O)=O)CC3)c(-c3ccc(F)c(C(=O)O)c3)cc2S1(=O)=O] |
| 537 | 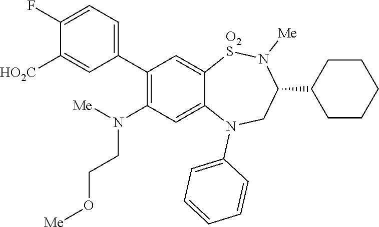 [SMILES: COCCN(C)c1cc2c(cc1-c1ccc(F)c(C(=O)O)c1)S(=O)(=O)N(C)[C@H](C1CCCCC1)CN2c1ccccc1] |
| 538 | 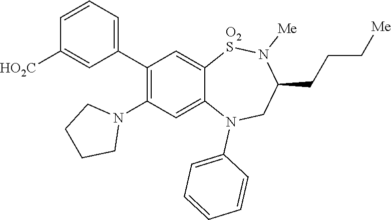 [SMILES: CCCC[C@H]1CN(c2ccccc2)c2cc(N3CCCC3)c(-c3cccc(C(=O)O)c3)cc2S(=O)(=O)N1C] |
| 539 | 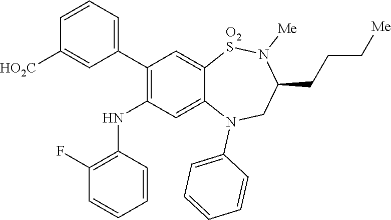 [SMILES: CCCC[C@H]1CN(c2ccccc2)c2cc(Nc3ccccc3F)c(-c3cccc(C(=O)O)c3)cc2S(=O)(=O)N1C] |
| 540 | 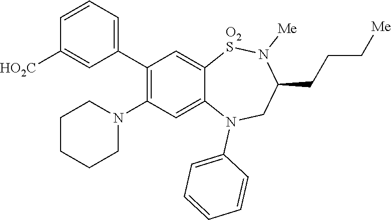 [SMILES: CCCC[C@H]1CN(c2ccccc2)c2cc(N3CCCCC3)c(-c3cccc(C(=O)O)c3)cc2S(=O)(=O)N1C] |
| 541 | 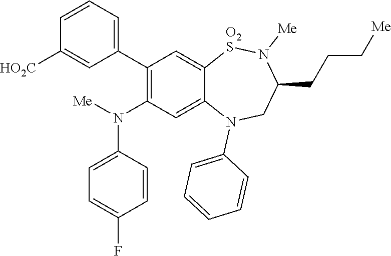 [SMILES: CCCC[C@H]1CN(c2ccccc2)c2cc(N(C)c3ccc(F)cc3)c(-c3cccc(C(=O)O)c3)cc2S(=O)(=O)N1C] |
| 542 | 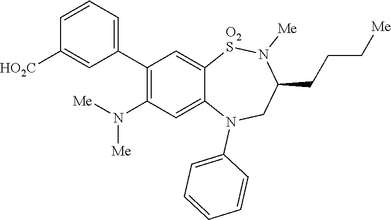 [SMILES: CCCC[C@H]1CN(c2ccccc2)c2cc(N(C)C)c(-c3cccc(C(=O)O)c3)cc2S(=O)(=O)N1C] |
| 543 | 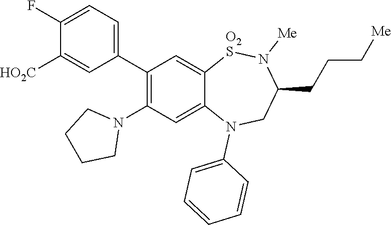 [SMILES: CCCC[C@H]1CN(c2ccccc2)c2cc(N3CCCC3)c(-c3ccc(F)c(C(=O)O)c3)cc2S(=O)(=O)N1C] |
| 544 | 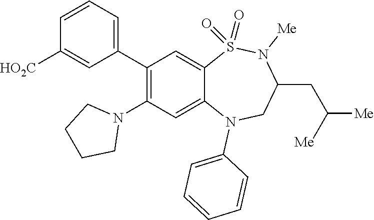 [SMILES: CC(C)CC1CN(c2ccccc2)c2cc(N3CCCC3)c(-c3cccc(C(=O)O)c3)cc2S(=O)(=O)N1C] |
| 545 | 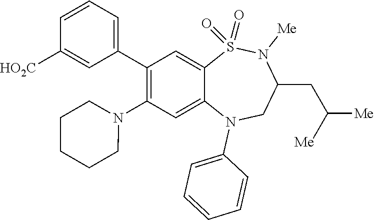 [SMILES: CC(C)CC1CN(c2ccccc2)c2cc(N3CCCCC3)c(-c3cccc(C(=O)O)c3)cc2S(=O)(=O)N1C] |
| 546 | 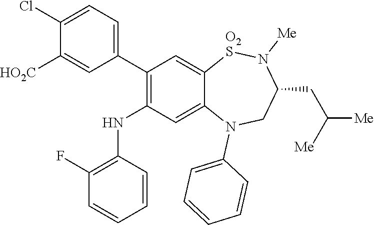 [SMILES: CC(C)C[C@@H]1CN(c2ccccc2)c2cc(Nc3ccccc3F)c(-c3ccc(Cl)c(C(=O)O)c3)cc2S(=O)(=O)N1C] |
| 547 | 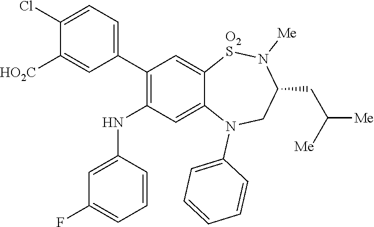 [SMILES: CC(C)C[C@@H]1CN(c2ccccc2)c2cc(Nc3cccc(F)c3)c(-c3ccc(Cl)c(C(=O)O)c3)cc2S(=O)(=O)N1C] |
| 548 | 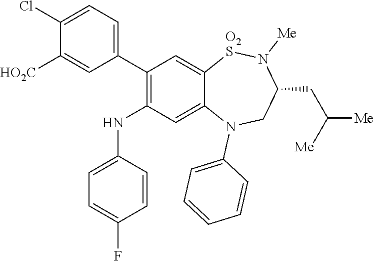 [SMILES: CC(C)C[C@@H]1CN(c2ccccc2)c2cc(Nc3ccc(F)cc3)c(-c3ccc(Cl)c(C(=O)O)c3)cc2S(=O)(=O)N1C] |
| 549 | 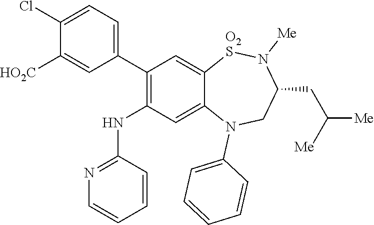 [SMILES: CC(C)C[C@@H]1CN(c2ccccc2)c2cc(Nc3ccccn3)c(-c3ccc(Cl)c(C(=O)O)c3)cc2S(=O)(=O)N1C] |
| 550 | 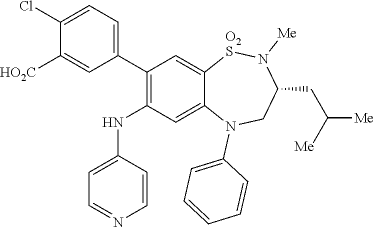 [SMILES: CC(C)C[C@@H]1CN(c2ccccc2)c2cc(Nc3ccncc3)c(-c3ccc(Cl)c(C(=O)O)c3)cc2S(=O)(=O)N1C] |
| 551 | 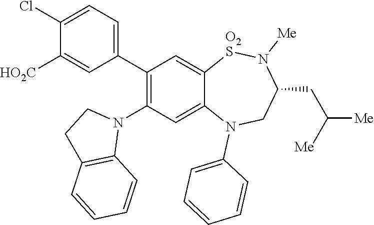 [SMILES: CC(C)C[C@@H]1CN(c2ccccc2)c2cc(N3CCc4ccccc43)c(-c3ccc(Cl)c(C(=O)O)c3)cc2S(=O)(=O)N1C] |
| 552 | 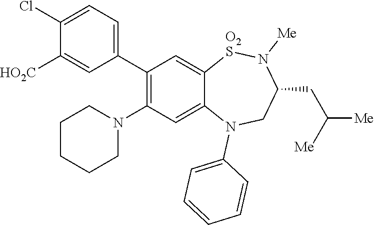 [SMILES: CC(C)C[C@@H]1CN(c2ccccc2)c2cc(N3CCCCC3)c(-c3ccc(Cl)c(C(=O)O)c3)cc2S(=O)(=O)N1C] |
| 553 | 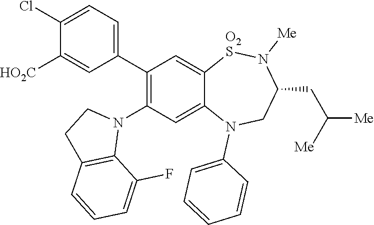 [SMILES: CC(C)C[C@@H]1CN(c2ccccc2)c2cc(N3CCc4cccc(F)c43)c(-c3ccc(Cl)c(C(=O)O)c3)cc2S(=O)(=O)N1C] |
| 554 | 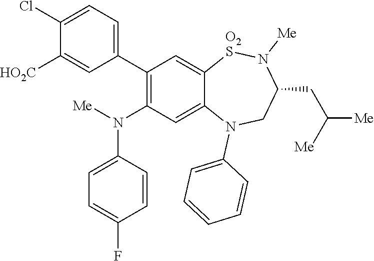 [SMILES: CC(C)C[C@@H]1CN(c2ccccc2)c2cc(N(C)c3ccc(F)cc3)c(-c3ccc(Cl)c(C(=O)O)c3)cc2S(=O)(=O)N1C] |
| 555 | 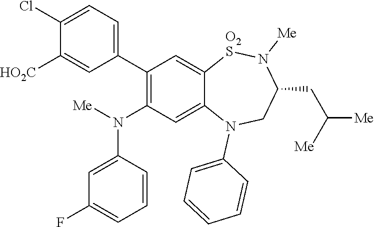 [SMILES: CC(C)C[C@@H]1CN(c2ccccc2)c2cc(N(C)c3cccc(F)c3)c(-c3ccc(Cl)c(C(=O)O)c3)cc2S(=O)(=O)N1C] |
| 556 | 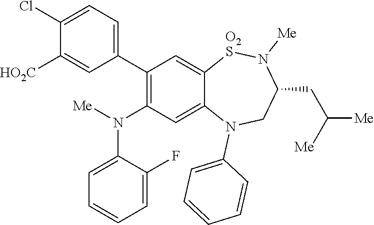 [SMILES: CC(C)C[C@@H]1CN(c2ccccc2)c2cc(N(C)c3ccccc3F)c(-c3ccc(Cl)c(C(=O)O)c3)cc2S(=O)(=O)N1C] |
| 557 | 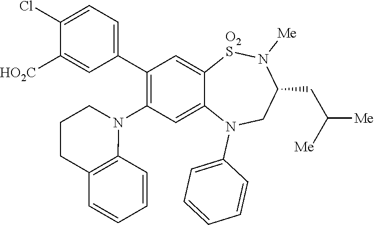 [SMILES: CC(C)C[C@@H]1CN(c2ccccc2)c2cc(N3CCCc4ccccc43)c(-c3ccc(Cl)c(C(=O)O)c3)cc2S(=O)(=O)N1C] |
| 558 | 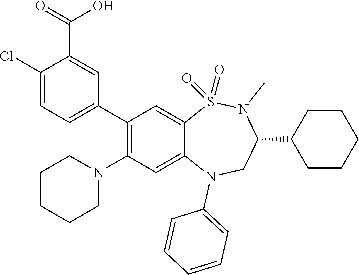 [SMILES: CN1[C@H](C2CCCCC2)CN(c2ccccc2)c2cc(N3CCCCC3)c(-c3ccc(Cl)c(C(=O)O)c3)cc2S1(=O)=O] |
| 559 | 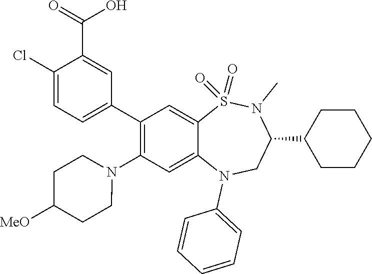 [SMILES: COC1CCN(c2cc3c(cc2-c2ccc(Cl)c(C(=O)O)c2)S(=O)(=O)N(C)[C@H](C2CCCCC2)CN3c2ccccc2)CC1] |
| 560 | 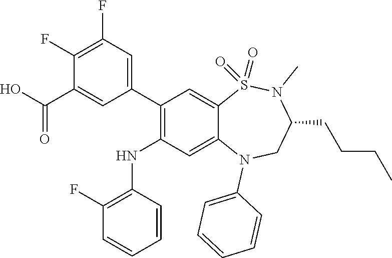 [SMILES: CCCC[C@@H]1CN(c2ccccc2)c2cc(Nc3ccccc3F)c(-c3cc(F)c(F)c(C(=O)O)c3)cc2S(=O)(=O)N1C] |
| 561 | 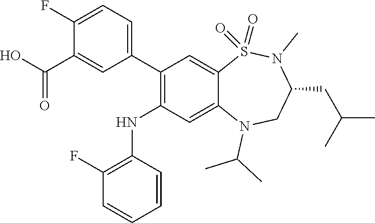 [SMILES: CC(C)C[C@@H]1CN(C(C)C)c2cc(Nc3ccccc3F)c(-c3ccc(F)c(C(=O)O)c3)cc2S(=O)(=O)N1C] |
| 562 | 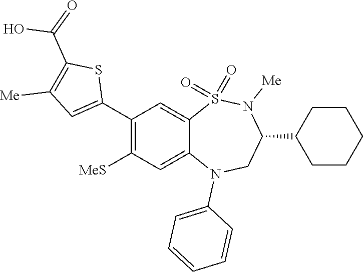 [SMILES: CSc1cc2c(cc1-c1cc(C)c(C(=O)O)s1)S(=O)(=O)N(C)[C@H](C1CCCCC1)CN2c1ccccc1] |
| 563 | 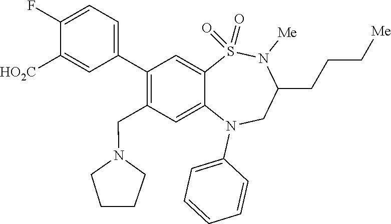 [SMILES: CCCCC1CN(c2ccccc2)c2cc(CN3CCCC3)c(-c3ccc(F)c(C(=O)O)c3)cc2S(=O)(=O)N1C] |
| 564 | 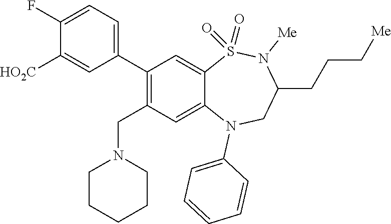 [SMILES: CCCCC1CN(c2ccccc2)c2cc(CN3CCCCC3)c(-c3ccc(F)c(C(=O)O)c3)cc2S(=O)(=O)N1C] |
| 565 | 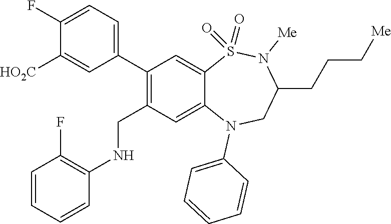 [SMILES: CCCCC1CN(c2ccccc2)c2cc(CNc3ccccc3F)c(-c3ccc(F)c(C(=O)O)c3)cc2S(=O)(=O)N1C] |
| 566 | 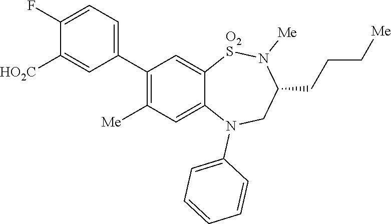 [SMILES: CCCC[C@@H]1CN(c2ccccc2)c2cc(C)c(-c3ccc(F)c(C(=O)O)c3)cc2S(=O)(=O)N1C] |
| 567 | 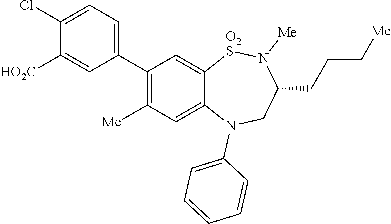 [SMILES: CCCC[C@@H]1CN(c2ccccc2)c2cc(C)c(-c3ccc(Cl)c(C(=O)O)c3)cc2S(=O)(=O)N1C] |
| 568 | 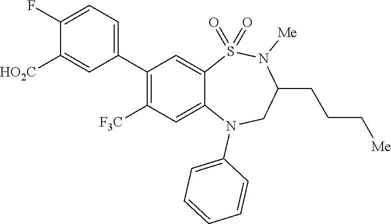 [SMILES: CCCCC1CN(c2ccccc2)c2cc(C(F)(F)F)c(-c3ccc(F)c(C(=O)O)c3)cc2S(=O)(=O)N1C] |
| 569 | 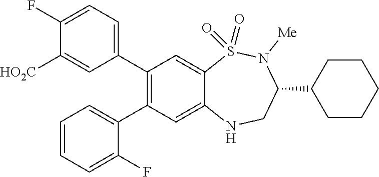 [SMILES: CN1[C@H](C2CCCCC2)CNc2cc(-c3ccccc3F)c(-c3ccc(F)c(C(=O)O)c3)cc2S1(=O)=O] |
| 570 | 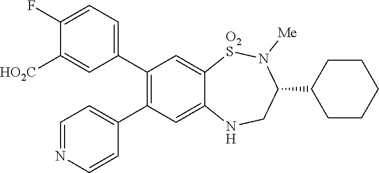 [SMILES: CN1[C@H](C2CCCCC2)CNc2cc(-c3ccncc3)c(-c3ccc(F)c(C(=O)O)c3)cc2S1(=O)=O] |
| 571 | 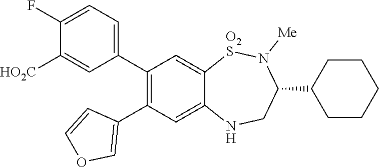 [SMILES: CN1[C@H](C2CCCCC2)CNc2cc(-c3ccoc3)c(-c3ccc(F)c(C(=O)O)c3)cc2S1(=O)=O] |
| 572 | 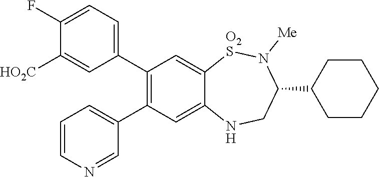 [SMILES: CN1[C@H](C2CCCCC2)CNc2cc(-c3cccnc3)c(-c3ccc(F)c(C(=O)O)c3)cc2S1(=O)=O] |
| 573 | 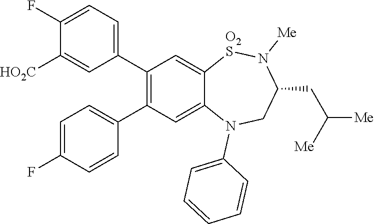 [SMILES: CC(C)C[C@@H]1CN(c2ccccc2)c2cc(-c3ccc(F)cc3)c(-c3ccc(F)c(C(=O)O)c3)cc2S(=O)(=O)N1C] |
| 574 | 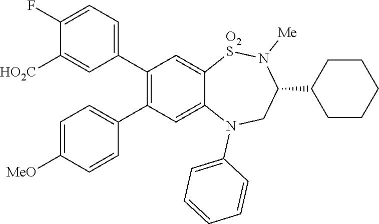 [SMILES: COc1ccc(-c2cc3c(cc2-c2ccc(F)c(C(=O)O)c2)S(=O)(=O)N(C)[C@H](C2CCCCC2)CN3c2ccccc2)cc1] |
| 575 | 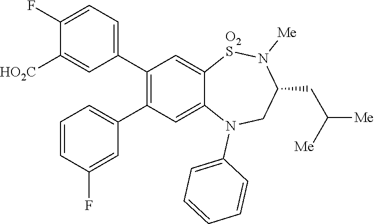 [SMILES: CC(C)C[C@@H]1CN(c2ccccc2)c2cc(-c3cccc(F)c3)c(-c3ccc(F)c(C(=O)O)c3)cc2S(=O)(=O)N1C] |
| 576 | 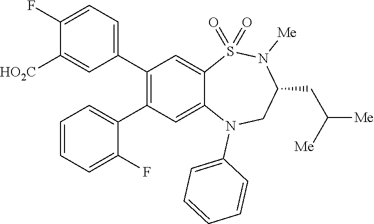 [SMILES: CC(C)C[C@@H]1CN(c2ccccc2)c2cc(-c3ccccc3F)c(-c3ccc(F)c(C(=O)O)c3)cc2S(=O)(=O)N1C] |
| 577 | 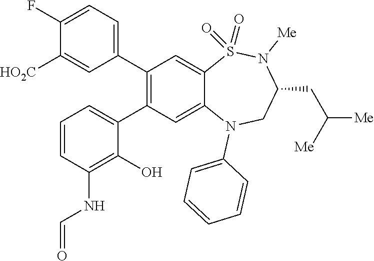 [SMILES: CC(C)C[C@@H]1CN(c2ccccc2)c2cc(-c3cccc(NC=O)c3O)c(-c3ccc(F)c(C(=O)O)c3)cc2S(=O)(=O)N1C] |
| 578 | 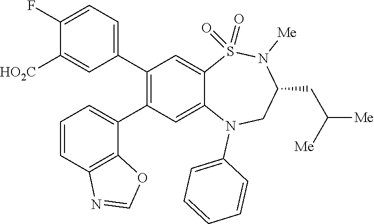 [SMILES: CC(C)C[C@@H]1CN(c2ccccc2)c2cc(-c3cccc4ncoc34)c(-c3ccc(F)c(C(=O)O)c3)cc2S(=O)(=O)N1C] |
| 579 | 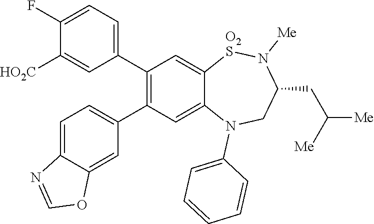 [SMILES: CC(C)C[C@@H]1CN(c2ccccc2)c2cc(-c3ccc4ncoc4c3)c(-c3ccc(F)c(C(=O)O)c3)cc2S(=O)(=O)N1C] |
| 580 | 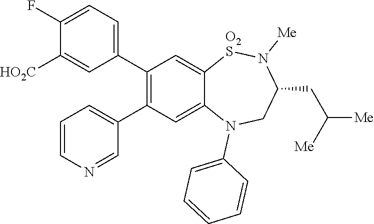 [SMILES: CC(C)C[C@@H]1CN(c2ccccc2)c2cc(-c3cccnc3)c(-c3ccc(F)c(C(=O)O)c3)cc2S(=O)(=O)N1C] |
| 581 | 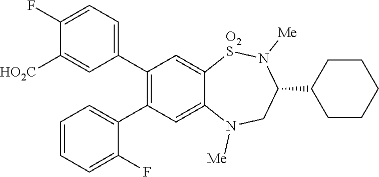 [SMILES: CN1C[C@@H](C2CCCCC2)N(C)S(=O)(=O)c2cc(-c3ccc(F)c(C(=O)O)c3)c(-c3ccccc3F)cc21] |
| 582 | 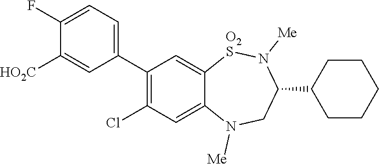 [SMILES: CN1C[C@@H](C2CCCCC2)N(C)S(=O)(=O)c2cc(-c3ccc(F)c(C(=O)O)c3)c(Cl)cc21] |
| 583 | 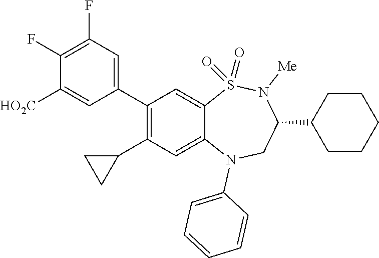 [SMILES: CN1[C@H](C2CCCCC2)CN(c2ccccc2)c2cc(C3CC3)c(-c3cc(F)c(F)c(C(=O)O)c3)cc2S1(=O)=O] |
| 584 | 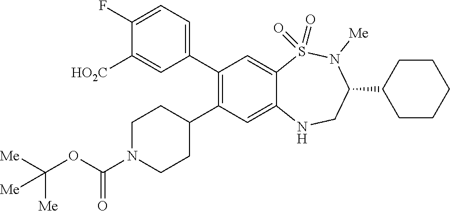 [SMILES: CN1[C@H](C2CCCCC2)CNc2cc(C3CCN(C(=O)OC(C)(C)C)CC3)c(-c3ccc(F)c(C(=O)O)c3)cc2S1(=O)=O] |
| 585 | 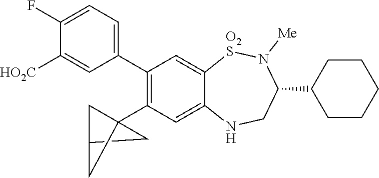 [SMILES: CN1[C@H](C2CCCCC2)CNc2cc(C34CC(C3)C4)c(-c3ccc(F)c(C(=O)O)c3)cc2S1(=O)=O] |
| 586 | 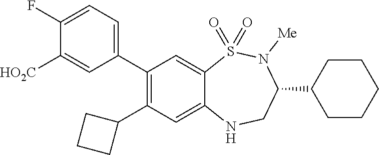 [SMILES: CN1[C@H](C2CCCCC2)CNc2cc(C3CCC3)c(-c3ccc(F)c(C(=O)O)c3)cc2S1(=O)=O] |
| 587 | 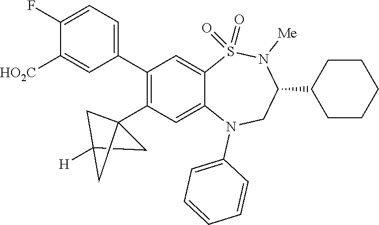 [SMILES: CN1[C@H](C2CCCCC2)CN(c2ccccc2)c2cc(C34CC(C3)C4)c(-c3ccc(F)c(C(=O)O)c3)cc2S1(=O)=O] |
| 588 | 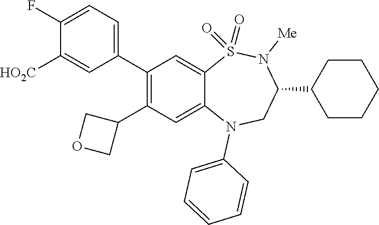 [SMILES: CN1[C@H](C2CCCCC2)CN(c2ccccc2)c2cc(C3COC3)c(-c3ccc(F)c(C(=O)O)c3)cc2S1(=O)=O] |
| 589 | 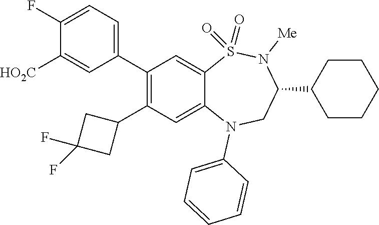 [SMILES: CN1[C@H](C2CCCCC2)CN(c2ccccc2)c2cc(C3CC(F)(F)C3)c(-c3ccc(F)c(C(=O)O)c3)cc2S1(=O)=O] |
| 590 | 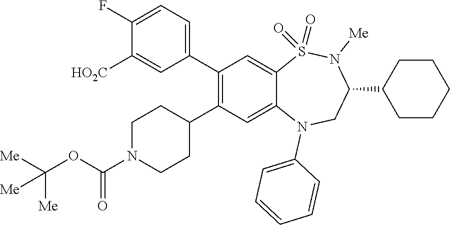 [SMILES: CN1[C@H](C2CCCCC2)CN(c2ccccc2)c2cc(C3CCN(C(=O)OC(C)(C)C)CC3)c(-c3ccc(F)c(C(=O)O)c3)cc2S1(=O)=O] |
| 591 | 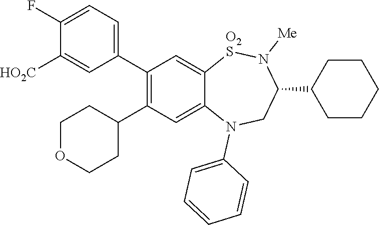 [SMILES: CN1[C@H](C2CCCCC2)CN(c2ccccc2)c2cc(C3CCOCC3)c(-c3ccc(F)c(C(=O)O)c3)cc2S1(=O)=O] |
| 592 | 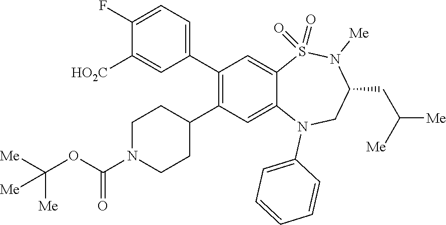 [SMILES: CC(C)C[C@@H]1CN(c2ccccc2)c2cc(C3CCN(C(=O)OC(C)(C)C)CC3)c(-c3ccc(F)c(C(=O)O)c3)cc2S(=O)(=O)N1C] |
| 593 | 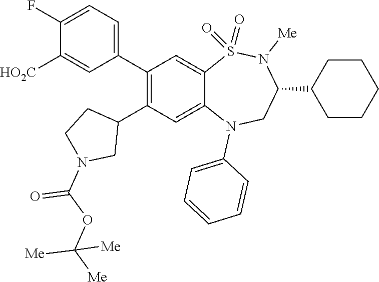 [SMILES: CN1[C@H](C2CCCCC2)CN(c2ccccc2)c2cc(C3CCN(C(=O)OC(C)(C)C)C3)c(-c3ccc(F)c(C(=O)O)c3)cc2S1(=O)=O] |
| 594 | 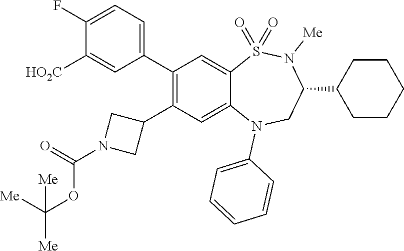 [SMILES: CN1[C@H](C2CCCCC2)CN(c2ccccc2)c2cc(C3CN(C(=O)OC(C)(C)C)C3)c(-c3ccc(F)c(C(=O)O)c3)cc2S1(=O)=O] |
| 595 | 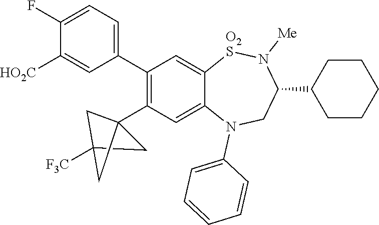 [SMILES: CN1[C@H](C2CCCCC2)CN(c2ccccc2)c2cc(C34CC(C(F)(F)F)(C3)C4)c(-c3ccc(F)c(C(=O)O)c3)cc2S1(=O)=O] |
| 596 | 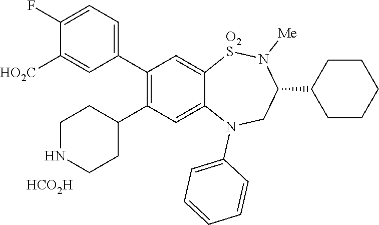 [SMILES: "HCO2H" ; CN1[C@H](C2CCCCC2)CN(c2ccccc2)c2cc(C3CCNCC3)c(-c3ccc(F)c(C(=O)O)c3)cc2S1(=O)=O] |
| 597 | 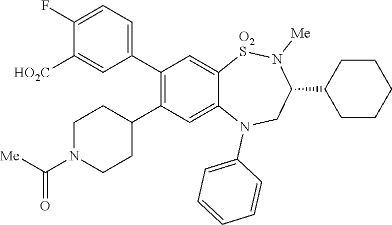 [SMILES: CC(=O)N1CCC(c2cc3c(cc2-c2ccc(F)c(C(=O)O)c2)S(=O)(=O)N(C)[C@H](C2CCCCC2)CN3c2ccccc2)CC1] |
| 598 | 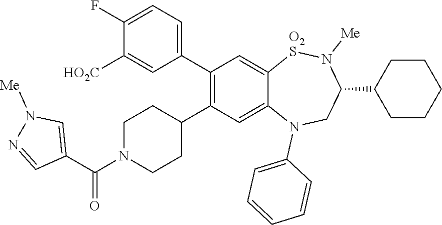 [SMILES: CN1[C@H](C2CCCCC2)CN(c2ccccc2)c2cc(C3CCN(C(=O)c4cnn(C)c4)CC3)c(-c3ccc(F)c(C(=O)O)c3)cc2S1(=O)=O] |
| 599 | 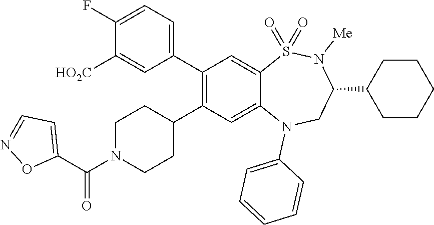 [SMILES: CN1[C@H](C2CCCCC2)CN(c2ccccc2)c2cc(C3CCN(C(=O)c4ccno4)CC3)c(-c3ccc(F)c(C(=O)O)c3)cc2S1(=O)=O] |
| 600 | 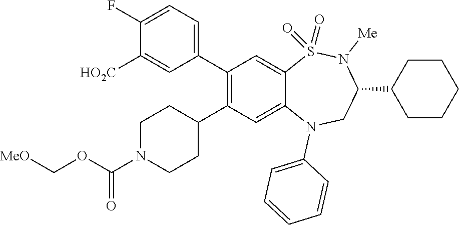 [SMILES: COCOC(=O)N1CCC(c2cc3c(cc2-c2ccc(F)c(C(=O)O)c2)S(=O)(=O)N(C)[C@H](C2CCCCC2)CN3c2ccccc2)CC1] |
| 601 | 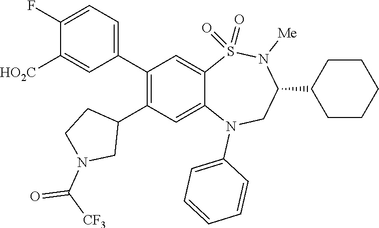 [SMILES: CN1[C@H](C2CCCCC2)CN(c2ccccc2)c2cc(C3CCN(C(=O)C(F)(F)F)C3)c(-c3ccc(F)c(C(=O)O)c3)cc2S1(=O)=O] |
| 602 | 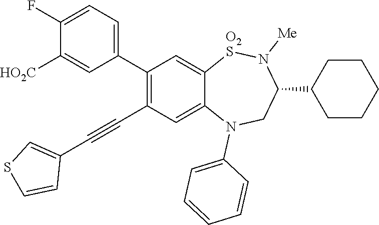 [SMILES: CN1[C@H](C2CCCCC2)CN(c2ccccc2)c2cc(C#Cc3ccsc3)c(-c3ccc(F)c(C(=O)O)c3)cc2S1(=O)=O] |
| 603 | 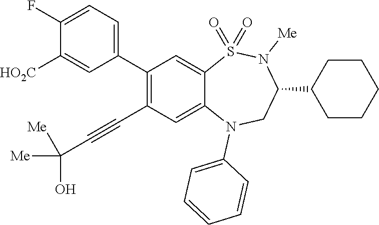 [SMILES: CN1[C@H](C2CCCCC2)CN(c2ccccc2)c2cc(C#CC(C)(C)O)c(-c3ccc(F)c(C(=O)O)c3)cc2S1(=O)=O] |
| 604 | 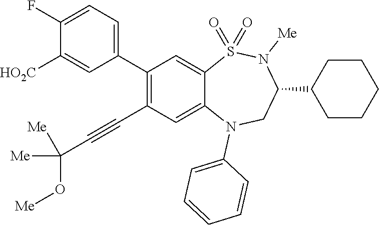 [SMILES: COC(C)(C)C#Cc1cc2c(cc1-c1ccc(F)c(C(=O)O)c1)S(=O)(=O)N(C)[C@H](C1CCCCC1)CN2c1ccccc1] |
| 605 | 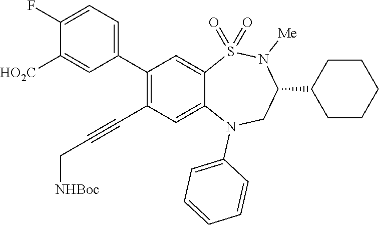 [SMILES: CN1[C@H](C2CCCCC2)CN(c2ccccc2)c2cc(C#CCNC(=O)OC(C)(C)C)c(-c3ccc(F)c(C(=O)O)c3)cc2S1(=O)=O] |
| 606 | 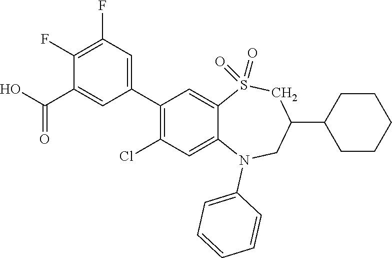 [SMILES: O=C(O)c1cc(-c2cc3c(cc2Cl)N(c2ccccc2)CC(C2CCCCC2)CS3(=O)=O)cc(F)c1F] |
| 607 | 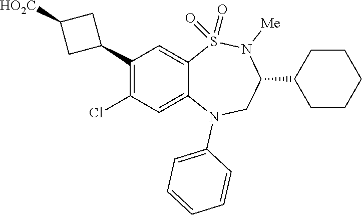 [SMILES: CN1[C@H](C2CCCCC2)CN(c2ccccc2)c2cc(Cl)c([C@H]3C[C@@H](C(=O)O)C3)cc2S1(=O)=O] |
| 608 | 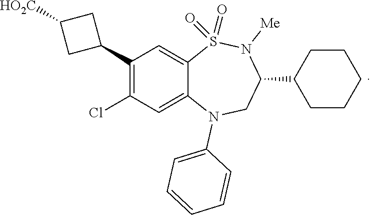 [SMILES: CN1[C@H](C2CCCCC2)CN(c2ccccc2)c2cc(Cl)c([C@H]3C[C@H](C(=O)O)C3)cc2S1(=O)=O] |

8. A pharmaceutical composition, comprising a compound according to claim 1, in combination with a pharmaceutically acceptable carrier or excipient.

9. A method of treating or preventing an HBV and/or HDV infection in a subject in need thereof, comprising administering to the subject a therapeutically effective amount of a compound or a combination of compounds according to claim 1.

10. The method of claim 9, further comprising administering to the subject an additional therapeutic agent selected from the group consisting of a HBV polymerase inhibitor, interferon, viral entry inhibitor, viral maturation inhibitor, literature-described capsid assembly modulator, reverse transcriptase inhibitor, TLR-agonist, inducer of cellular viral RNA sensor, therapeutic vaccine, and agents of distinct or unknown mechanism, and a combination thereof.

11. The method of claim 10, wherein the compound and the additional therapeutic agent are co-formulated.

12. The method of claim 10, wherein the compound and the additional therapeutic agent are co-administered.

13. The method of claim 10, wherein the additional therapeutic agent is administered at a lower dose or frequency compared to the dose or frequency of the additional therapeutic agent that is required to treat an HBV and/or HDV infection when administered alone.

14. The method of claim 10, wherein the subject is refractory to at least one compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

15. The method of claim 10, wherein the method reduces viral load in the subject to a greater extent compared to the administering of a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

16. The method of claim 10, wherein the method results in a lower incidence of viral mutation and/or viral resistance than the treatment with a compound selected from the group consisting of a HBV polymerase inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.
